(19) United States
(12) Patent Application Publication
(10) Pub. No.: US 2025/0331414 A1
(43) Pub. Date: Oct. 23, 2025

(54) HETEROCYCLIC COMPOUND, ORGANIC ELECTROLUMINESCENT DEVICE AND ELECTRONIC APPARATUS

(71) Applicant: Shaanxi Lighte Optoelectronics Material Co., Ltd., Xi'an (CN)

(72) Inventors: Xianbin XU, Xi'an (CN); Lei YANG, Xi'an (CN)

(21) Appl. No.: 18/286,793

(22) PCT Filed: Mar. 13, 2023
(86) PCT No.: PCT/CN2023/081182

§ 371 Date: Oct. 13, 2023

(30) Foreign Application Priority Data
Jun. 13, 2022 (CN) 202210662931.2

(51) Int. Cl. H10K 85/60; C07D 498/04; H10K 50/11; H10K 50/15

(52) U.S. Cl. CPC H10K 85/636; C07D 498/04; H10K 85/633; H10K 50/11; H10K 50/156; H10K 85/615; H10K 85/622; H10K 85/657; H10K 85/6572; H10K 85/6574; H10K 85/6576

(57) ABSTRACT

The present application relates to the technical field of organic electroluminescent materials, and provides a heterocyclic compound, an organic electroluminescent device and an electronic apparatus comprising the heterocyclic compound. The heterocyclic compound of the present application includes a parent nucleus structure of naphthofuranoxazole/thiazole and triarylamine. When the compound is used as a host material or hole adjustment layer of the organic electroluminescent device, the electroluminescent efficiency and life of the device can be significantly improved.

### CROSS REFERENCE TO RELATED APPLICATIONS

The present application claims priority to Chinese Patent Application No. CN202210662931.2 filed on Jun. 13, 2022, the entire content of which is incorporated herein by reference as a part of the application.

### FIELD

The present application relates to the technical field of organic electroluminescent materials, in particular to a heterocyclic compound and an organic electroluminescent device and an electronic apparatus comprising the heterocyclic compound.

### BACKGROUND

With the development of electronic technology and the progress of material science, the application range of electronic components used to achieve electroluminescence or photoelectric conversion is increasingly widespread. An organic electroluminescent device (OLED) usually includes: a cathode and an anode disposed oppositely, and a functional layer disposed between the cathode and the anode. The functional layer consists of a plurality of organic or inorganic film layers, and generally includes an organic luminescent layer, a hole transport layer, an electron transport layer, and the like. When voltage is applied to the cathode and the anode, the two electrodes generate an electric field. Under the effect of the electric field, electrons on the cathode side move to the organic luminescent layer, holes on the anode side also move to the organic luminescent layer, the electrons and the holes combine in the organic luminescent layer to form excitons, and the excitons release energy outward in an excited state, so that the organic luminescent layer emits light to the outside.

Among the existing organic electroluminescent device, major problems are reflected in life span and efficiency. With the large-area development of displays, driving voltage also increases, and luminous efficiency and current efficiency also need to be improved. Therefore, it is necessary to continue to develop novel materials to further improve the performance of the organic electroluminescent device.

### SUMMARY

In view of the above problems existing in the prior art, the present application aims to provide a heterocyclic compound, an organic electroluminescent device and an electronic apparatus comprising the heterocyclic compound, where the heterocyclic compound is used in the organic electroluminescent device to improve the performance of the device.

A first aspect of the present application provides a heterocyclic compound having a structure as shown in Formula 1.

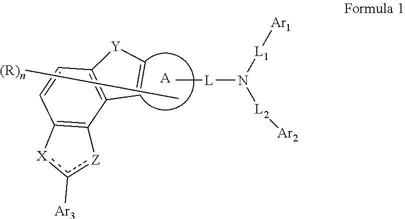

where Y is selected from S or O;
one of X and Z is —N═, and the other is O or S;
Ring A is selected from a naphthalene ring or a phenanthrene ring;
L, L₁, and L₂ are the same or different, and are each independently selected from a single bond, a substituted or unsubstituted arylene with 6 to 30 carbon atoms, or a substituted or unsubstituted heteroarylene with 3 to 30 carbon atoms;
Ar₁, Ar₂, and Ar₃ are the same or different, and are each independently selected from a substituted or unsubstituted aryl with 6 to 40 carbon atoms, a substituted or unsubstituted heteroaryl with 3 to 40 carbon atoms, an alkyl with 1 to 10 carbon atoms, or a cycloalkyl with 3 to 10 carbon atoms;
substituents of L, L₁, L₂, Ar₁, Ar₂, and Ar₃ are the same or different, and are each independently selected from deuterium, a cyano, a halogen group, an alkyl with 1 to 10 carbon atoms, a haloalkyl with 1 to 10 carbon atoms, a deuteroalkyl with 1 to 10 carbon atoms, a trialkylsilyl with 3 to 12 carbon atoms, a triphenylsilyl, an aryl with 6 to 20 carbon atoms, a deuteroaryl with 6 to 20 carbon atoms, a heteroaryl with 3 to 20 carbon atoms, a cycloalkyl with 3 to 10 carbon atoms, an alkoxy with 1 to 10 carbon atoms, an alkylthio with 1 to 10 carbon atoms, an aryloxy with 6 to 20 carbon atoms, or an arylthio with 6 to 20 carbon atoms; optionally, any two adjacent substituents form a saturated or unsaturated 3-membered to 15-membered ring; and
each R is the same or different, and is each independently selected from hydrogen, deuterium, a cyano, a halogen group, an alkyl with 1 to 10 carbon atoms, a haloalkyl with 1 to 10 carbon atoms, a deuteroalkyl with 1 to 10 carbon atoms, a trialkylsilyl with 3 to 12 carbon atoms, a triphenylsilyl, an aryl with 6 to 20 carbon atoms, a heteroaryl with 3 to 20 carbon atoms, or a cycloalkyl with 3 to 10 carbon atoms; optionally, any two adjacent Rs form a ring; and n is selected from 1, 2, 3, 4, 5, 6, 7, 8, or 9.

A second aspect of the present application provides an organic electroluminescent device including an anode and a cathode disposed oppositely, and a functional layer disposed between the anode and the cathode, where the functional layer includes the above-mentioned heterocyclic compound.

A third aspect of the present application provides an electronic apparatus including the organic electroluminescent device described in the second aspect.

The compound of the present application includes naphthofurano (phenanthrofurano) oxazole/thiazole and triarylamine, where arylamine is connected to a naphthalene ring of a naphthofuran (phenanthrofuran) group. After naphthofuran (phenanthrofuran) is condensed with oxazole/thiazole, a conjugated system of the compound increases, which helps to stack molecules, thereby significantly enhancing hole transport capacity of the compound of the present application. The compound of the present application is mixed with an electron transport material to form a mixed host material, which can improve carrier balance in the luminescent layer, broaden a carrier recombination region, improve exciton generation and utilization efficiency, improve electroluminescent efficiency of the device, and prolong service life of the device.

### BRIEF DESCRIPTION OF THE DRAWINGS

The accompanying drawings, which are intended to provide a further understanding of the present application and constitute a part of the description, are used together with specific embodiments below to explain the present application, but do not constitute limitations on the present application.

FIG. 1 is a schematic structural diagram of an organic electroluminescent device according to an embodiment of the present application.

FIG. 2 is a schematic structural diagram of an electronic apparatus according to an embodiment of the present application.

### REFERENCE NUMERALS

100. anode; 200. cathode; 300. functional layer; 310. hole injection layer; 321. hole transport layer; 322. hole adjustment layer; 330. organic luminescent layer; 340. electron transport layer; 350. electron injection layer; 400. electronic apparatus.

### DETAILED DESCRIPTION

Exemplary embodiments will now be described more comprehensively with reference to the accompanying drawings. However, the exemplary embodiments can be implemented in multiple forms and should not be interpreted as being limited to the examples elaborated herein. On the contrary, these embodiments are provided to make the present application more comprehensive and complete, and to comprehensively convey the concept of the exemplary embodiments to those skilled in the art. The described features, structures, or characteristics may be combined in one or more embodiments in any suitable manner. In the following description, many specific details are provided to provide a full understanding of the embodiments of the present application.

In a first aspect, the present application provides a heterocyclic compound having a structure as shown in Formula 1:

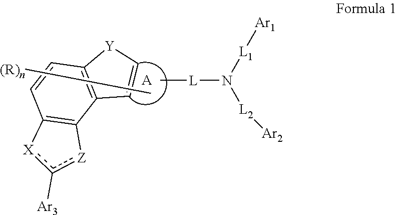

where Y is selected from S or O;
one of X and Z is —N═, and the other is O or S;
Ring A is selected from a naphthalene ring or a phenanthrene ring;
L, L₁, and L₂ are the same or different, and are each independently selected from a single bond, a substituted or unsubstituted arylene with 6 to 30 carbon atoms, or a substituted or unsubstituted heteroarylene with 3 to 30 carbon atoms;
Ar₁, Ar₂, and Ar₃ are the same or different, and are each independently selected from a substituted or unsubstituted aryl with 6 to 40 carbon atoms, a substituted or unsubstituted heteroaryl with 3 to 40 carbon atoms, an alkyl with 1 to 10 carbon atoms, or a cycloalkyl with 3 to 10 carbon atoms;
substituents of L, L₁, L₂, Ar₁, Ar₂, and Ar₃ are the same or different, and are each independently selected from deuterium, a cyano, a halogen group, an alkyl with 1 to 10 carbon atoms, a haloalkyl with 1 to 10 carbon atoms, a deuteroalkyl with 1 to 10 carbon atoms, a trialkylsilyl with 3 to 12 carbon atoms, a triphenylsilyl, an aryl with 6 to 20 carbon atoms, a deuteroaryl with 6 to 20 carbon atoms, a heteroaryl with 3 to 20 carbon atoms, a cycloalkyl with 3 to 10 carbon atoms, an alkoxy with 1 to 10 carbon atoms, an alkylthio with 1 to 10 carbon atoms, an aryloxy with 6 to 20 carbon atoms, or an arylthio with 6 to 20 carbon atoms; optionally, any two adjacent substituents form a saturated or unsaturated 3-membered to 15-membered ring; and
each R is the same or different, and is each independently selected from hydrogen, deuterium, a cyano, a halogen group, an alkyl with 1 to 10 carbon atoms, a haloalkyl with 1 to 10 carbon atoms, a deuteroalkyl with 1 to 10 carbon atoms, a trialkylsilyl with 3 to 12 carbon atoms, a triphenylsilyl, an aryl with 6 to 20 carbon atoms, a heteroaryl with 3 to 20 carbon atoms, or a cycloalkyl with 3 to 10 carbon atoms; optionally, any two adjacent Rs form a ring; and n is selected from 1, 2, 3, 4, 5, 6, 7, 8, or 9.

In the present application, the terms “optional” and “optionally” mean that the event or circumstances described subsequently may or may not occur. For example, “optionally, in Ar₁, Ar₂, and Ar, any two adjacent substituents form a saturated or unsaturated 3-membered to 15-membered ring” includes a scenario where any two adjacent substituents form a ring and a scenario where any two adjacent substituents exist independently and do not form a ring. “Any two adjacent” may include two substituents on the same atom or one substituent on each of two adjacent atoms. When there are two substituents on the same atom, the two substituents may form a saturated or unsaturated spiral ring with atoms connected to the two substituents. When there is one substituent on each of two adjacent atoms, the two substituents may be condensed into a ring.

In the present application, the descriptions “each . . . independently”, “ . . . respectively and independently”, and “each independently” are interchangeable and should be understood in a broad sense. Such descriptions may indicate that specific options expressed between the same symbols in different groups do not affect each other, or that specific options expressed between the same symbols in the same group do not affect each other. For example,

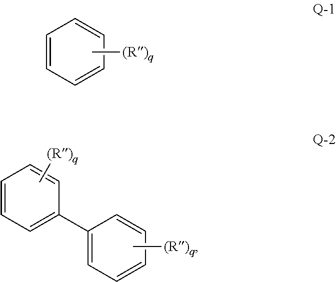

where each q is independently 0, 1, 2, or 3, and each R″ is independently selected from hydrogen, deuterium, fluorine, or chlorine″ means that formula Q-1 represents q substituent Rs″ on a benzene ring, each R″ may be the same or different, and options for each R″ do not affect each other; and formula Q-2 represents q substituent R″s on each benzene ring of biphenyl, the number q of substituent R″s on two benzene rings may be the same or different, each R″ may be the same or different, and options for each R″ do not affect each other.

In the present application, the term “substituted or unsubstituted” indicates that the functional group described after the term may have or may don't have a substituent (hereinafter referred to as Rc for ease of description). For example, “substituted or unsubstituted aryl” indicates aryl with a substituent Rc or unsubstituted aryl. The substituent Rc may be, for example, deuterium, a halogen group, a cyano, a heteroaryl, an aryl, a trialkylsilyl, an alkyl, a haloalkyl, a cycloalkyl, a deuterophenyl, and the like. The number of substituents may be one or more.

In the present application, “more” or “a plurality of” refers to two or more, such as two, three, four, five, six and the like.

In the present application, the carbon atoms of a substituted or unsubstituted functional group refer to all carbon atoms.

The present application mentions forming a ring, such as a saturated or unsaturated 3-membered to 15-membered ring, including a saturated carbon ring, a saturated heterocyclic ring, a partially unsaturated carbon ring, a partially unsaturated heterocyclic ring, an aromatic carbon ring, and an aromatic heterocyclic ring; and when n-membered is used as a prefix of a ring, n is an integer, indicating that the number of ring atoms in the ring is n. For example, the 3-membered to 15-membered ring represents a ring with 3 to 15 ring atoms, including 3, 4, 5, 6, 7, 8, 9, 10, 11, 12, 13, 14, or 15 ring atoms.

The hydrogen atom in the compound of the present application includes various isotope atoms of a hydrogen element, such as hydrogen (H), deuterium (D), or tritium (T).

In the present application, the aryl refers to an optional functional group or substituent derived from an aromatic carbon ring. The aryl may be monocyclic aryl (such as phenyl) or polycyclic aryl. That is, the aryl may be monocyclic aryl, fused aryl, two or more monocyclic aryls conjugated through carbon-carbon bonds, monocyclic aryl and fused aryl conjugated through carbon-carbon bonds, or two or more fused aryls conjugated through carbon-carbon bonds. That is, unless otherwise specified, two or more aromatic groups conjugated through carbon-carbon bonds may alternatively be considered as the aryl of the present application. The fused aryl may include, for example, bicyclic fused aryl (such as naphthyl), tricyclic fused aryl (such as phenyl, fluorenyl or anthracyl), or the like. The aryl does not contain heteroatoms such as B, N, O, S, P, Se, and Si. Examples of the aryl include, but are not limited to, phenyl, naphthyl, fluorenyl, spirodifluorenyl, anthracyl, phenanthryl, biphenyl, terphenyl, triphenylene

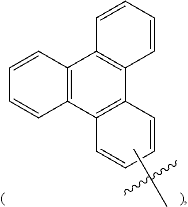

perylenyl, benzo[9,10]phenanthryl, pyrenyl, benzofluoranthenyl, chrysenyl, and the like.

In the present application, the arylene refers to a divalent group formed by further loss of one or more hydrogen atoms from the aryl.

In the present application, the terphenyl includes

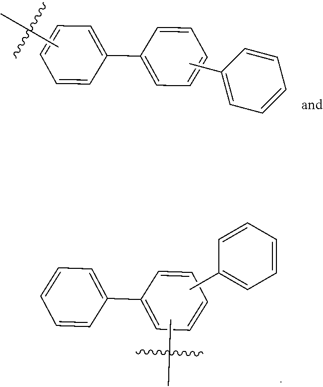

In the present application, the number of carbon atoms of the substituted or unsubstituted aryl (arylene) may be 6, 8, 10, 12, 13, 14, 15, 16, 17, 18, 19, 20, 21, 22, 23, 24, 25, 30, 31, 32, 33, 34, 35, 36, 37, 38, 39, or 40. In some embodiments, the substituted or unsubstituted aryl is a substituted or unsubstituted aryl with 6 to 30 carbon atoms. In other embodiments, the substituted or unsubstituted aryl is a substituted or unsubstituted aryl with 6 to 25 carbon atoms. In other embodiments, the substituted or unsubstituted aryl is a substituted or unsubstituted aryl with 6 to 18 carbon atoms. In other embodiments, the substituted or unsubstituted aryl is a substituted or unsubstituted aryl with 6 to 15 carbon atoms.

In the present application, the fluorenyl may be substituted for one or more substituents. In a case that the fluorenyl is substituted, the substituted fluorenyl may be

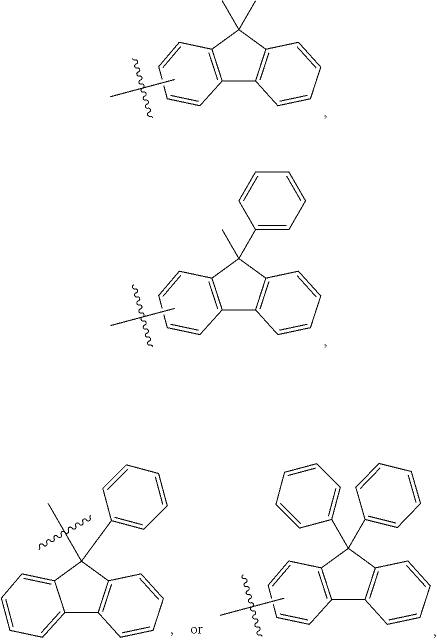

but is not limited thereto.

In the present application, the aryl as a substituent of L, L₁, L₂, Ar₃, Ar₁, and Ar₂ is for example, but not limited to, phenyl, naphthyl, phenanthryl, biphenyl, fluorenyl, dimethylfluorenyl, or the like.

In the present application, the heteroaryl is a univalent aromatic ring containing 1, 2, 3, 4, 5, or 6 heteroatoms or derivatives thereof, and the heteroatoms may be one or more of B, O, N, P, Si, Se, and S. The heteroaryl may be monocyclic heteroaryl or polycyclic heteroaryl. That is, the heteroaryl may be a single aromatic ring system, or a plurality of aromatic ring systems conjugated by carbon-carbon bonds, and any aromatic ring system is an aromatic single ring or an aromatic fused ring. For example, the heteroaryl may include, but is not limited to, thiophenyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolinyl, quinazolinyl, quinoxalinyl, phenoxazinyl, phthalazinyl, pyridinopyrimidinyl, pyridinopyrazinyl, pyrazinopyrizinyl, isoquinolinyl, indolyl, carbazolyl, benzoxazolyl, benzimidazolyl, benzothiazolyl, benzocarbazolyl, benzothiophenyl, dibenzothiophenyl, thienothiophenyl, benzofuryl, phenanthrolinyl, isoxazolyl, thiadiazolyl, phenothiazinyl, silafluorenyl, dibenzofuranyl, N-phenylcarbazolyl, N-pyridylcarbazolyl, N-methylcarbazolyl, or the like.

In the present application, the heteroarylene refers to a divalent or multivalent group formed by further loss of one or more hydrogen atoms from the heteroaryl.

In the present application, the number of carbon atoms of the substituted or unsubstituted heteroaryl (heteroarylene) may be 3, 4, 5, 6, 7, 8, 9, 10, 11, 12, 13, 14, 15, 16, 17, 18, 20, 21, 22, 23, 24, 25, 26, 27, 28, 29, 30, 31, 32, 33, 34, 35, 36, 37, 38, 39, or 40. In some embodiments, the substituted or unsubstituted heteroaryl is a substituted or unsubstituted heteroaryl with 3 to 30 carbon atoms. In other embodiments, the substituted or unsubstituted heteroaryl is a substituted or unsubstituted heteroaryl with 12 to 18 carbon atoms. In other embodiments, the substituted or unsubstituted heteroaryl is a substituted or unsubstituted heteroaryl with 5 to 12 carbon atoms.

In the present application, the heteroaryl as a substituent of L, L₁, L₂, Ar₃, Ar₁, and Ar₂ is, but not limited to, pyridyl, carbazolyl, dibenzothiophenyl, dibenzofuranyl, benzoxazolyl, benzothiazolyl, or benzimidazolyl.

In the present application, the substituted heteroaryl may be that one or more hydrogen atoms in heteroaryl are substituted by groups such as deuterium atom, halogen group, —CN, aryl, heteroaryl, trialkylsilyl, alkyl, cycloalkyl, haloalkyl, or the like.

In the present application, the alkyl with 1 to 10 carbon atoms may include linear alkyl with 1 to 10 carbon atoms and branched alkyl with 3 to 10 carbon atoms. The number of carbon atoms of the alkyl may be, for example, 1, 2, 3, 4, 5, 6, 7, 8, 9, or 10. Specific examples of the alkyl include, but are not limited to, methyl, ethyl, n-propyl, isopropyl, n-butyl, isobutyl, tert-butyl, n-pentyl, isopentyl, neopentyl, n-hexyl, and the like.

In the present application, the halogen group may be, for example, fluorine, chlorine, bromine, or iodine.

In the present application, specific examples of the trialkylsilyl include, but are not limited to, trimethylsilyl, triethylsilyl, and the like.

In the present application, the haloalkyl refers to alkyl with one or more halogen substituents, and a specific example includes, but is not limited to, trifluoromethyl.

In the present application, the number of carbon atoms of the cycloalkyl with 3 to 10 carbon atoms may be, for example, 3, 4, 5, 6, 7, 8, or 10. Specific examples of the cycloalkyl include, but are not limited to, cyclopentyl, cyclohexyl, or adamantyl.

In the present application, the number of carbon atoms of the deuteroalkyl with 1 to 10 carbon atoms is, for example, 1, 2, 3, 4, 5, 6, 7, 8, or 10. A specific example of the deuteroalkyl includes, but is not limited to, trideuteromethyl.

In the present application, the number of carbon atoms of the haloalkyl with 1 to 10 carbon atoms is, for example, 1, 2, 3, 4, 5, 6, 7, 8, or 10. A specific example of the haloalkyl includes, but is not limited to, trifluoromethyl.

In the present application, the ring system formed by n atoms is an n-membered ring. For example, the phenyl has a 6-membered ring. The 3-membered to 15-membered ring refers to a ring group with 3 to 15 ring atoms. The 3-membered to 15-membered ring is, for example, a cyclopentane, a cyclohexane, a fluorene ring, a benzene ring, or the like.

In the present application, [symbol] refers to a chemical bond connected to other groups.

In the present application, the single bond “ [symbol] ” extending from the ring system involved in an unpositioned connecting bond represents that one end of the connecting bond may be connected to any position through which the bond runs in the ring system, and the other end may be connected to the rest of the compound molecule. For example, as shown in Formula (f), the naphthyl represented by Formula (f) is connected to other positions of a molecule through two unpositioned connecting bonds that run through double rings, including any possible connection as shown in Formulas (f-1) to (f-10):

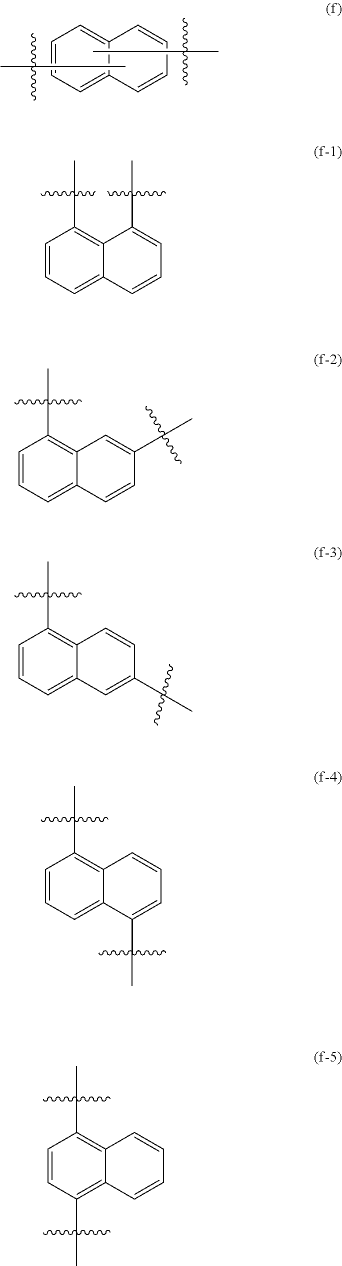

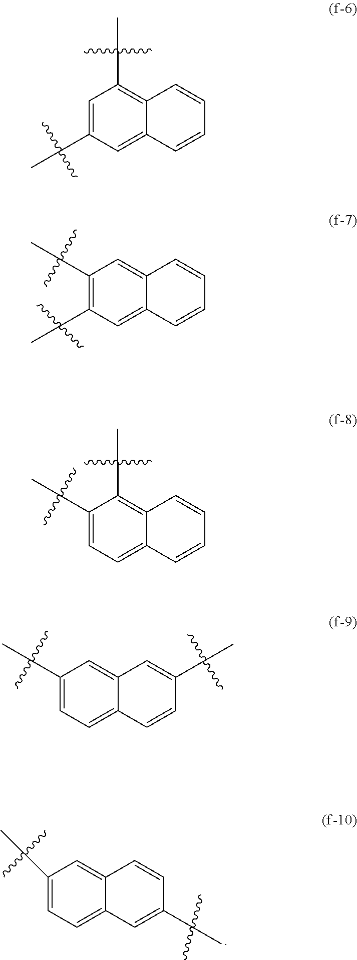 [SMILES: *c1cc(*)c2ccccc2c1 ; *c1cc2ccccc2cc1* ; *c1ccc2ccccc2c1* ; *c1ccc2ccc(*)cc2c1 ; *c1ccc2cc(*)ccc2c1]

For another example, as shown in Formula (X′) below, the dibenzofuranyl represented by Formula (X′) is connected to another position of a molecule through an unpositioned connecting bond extending from the centre of a benzene ring on one side, including any possible connection as shown in Formulas (X′-1) to (X′-4):

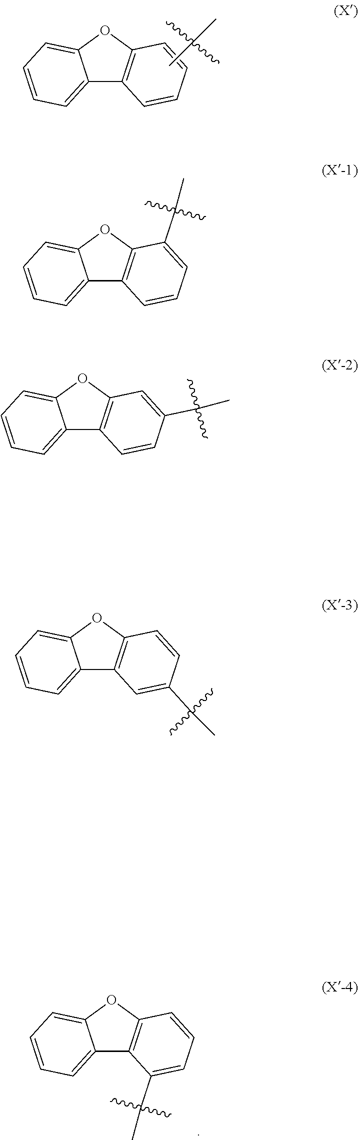

The unpositioned substituent in the present application refers to a substituent connected by a single bond extending from the centre of the ring system, which indicates that the substituent may be connected to any possible position in the ring system. For example, as shown in Formula (Y) below, the substituent R′ represented by Formula (Y) is connected to a quinoline ring through an unpositioned connecting bond, what it means, including any possible connection shown in Formulas (Y-1) to (Y-7):

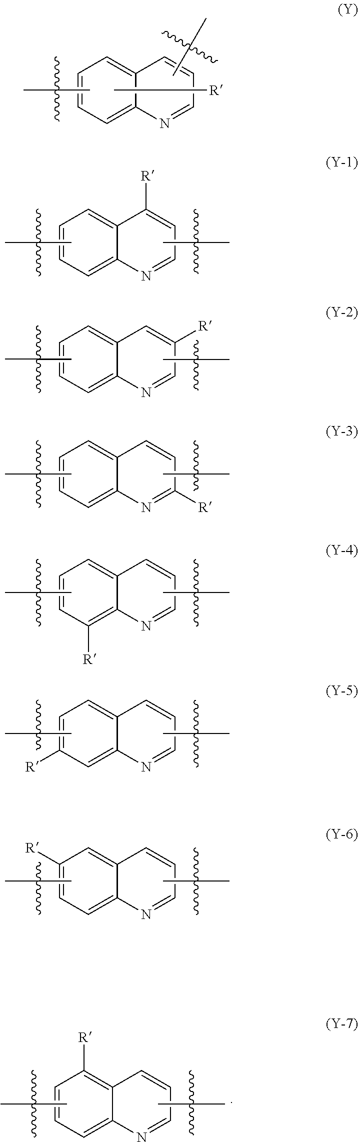

In some embodiments, the compound as shown in Formula 1 has structures as shown in Formulas 1-1 to 1-16:

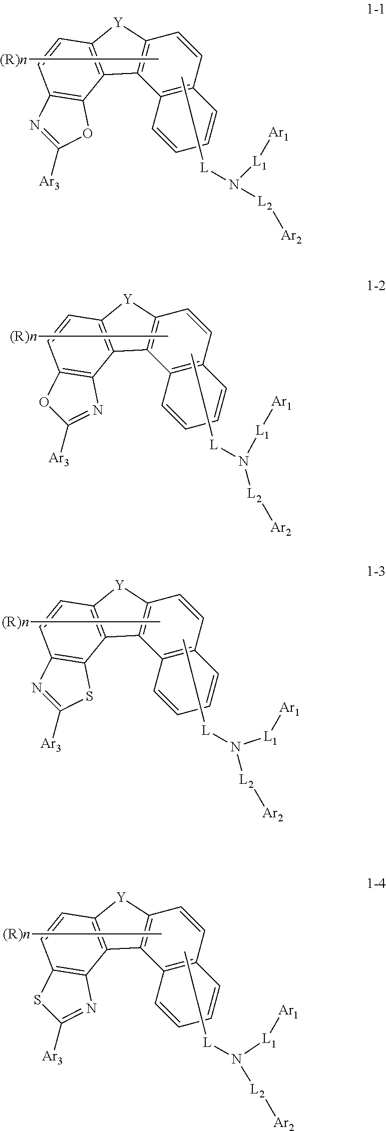

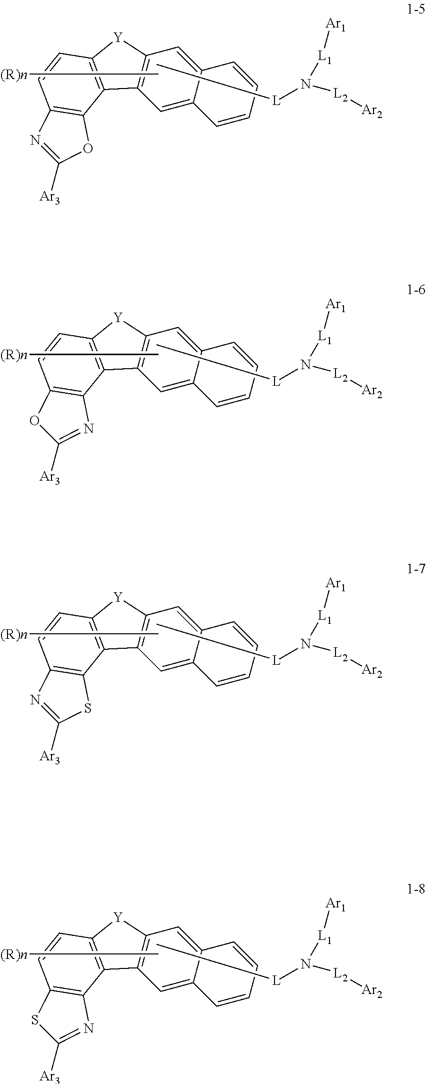

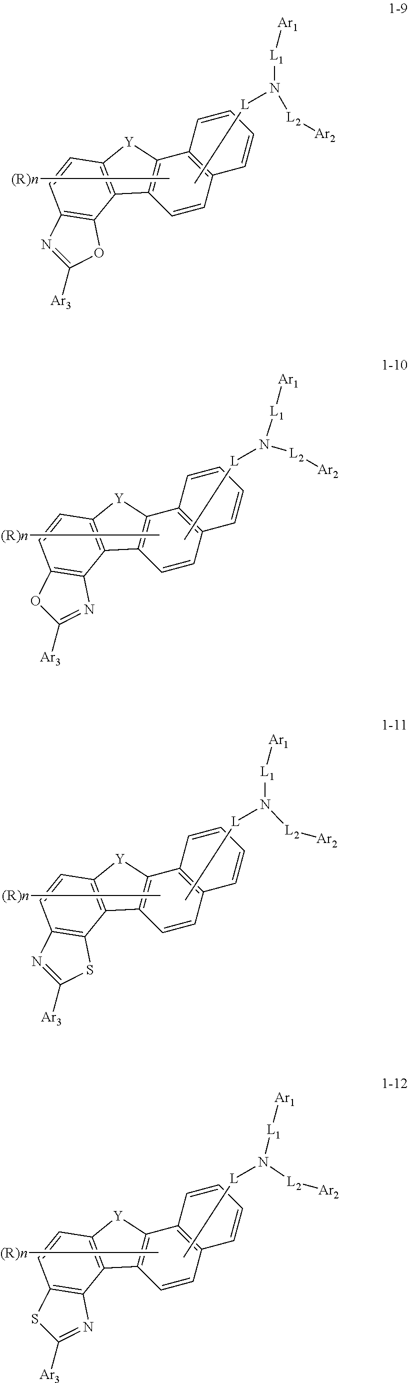

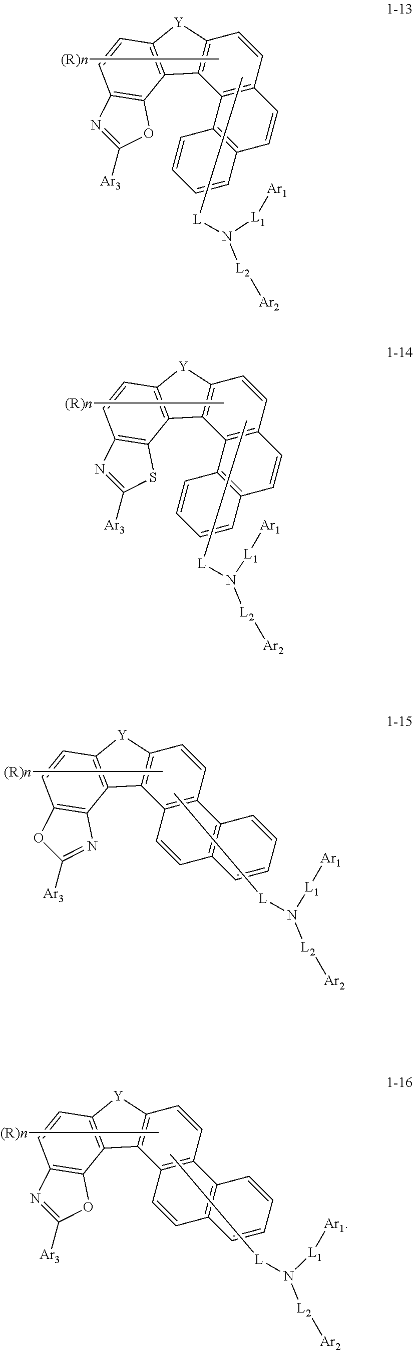

In some embodiments, the compound as shown in Formula 1 has structures as shown in Formulas (2-1) to (2-15) below:

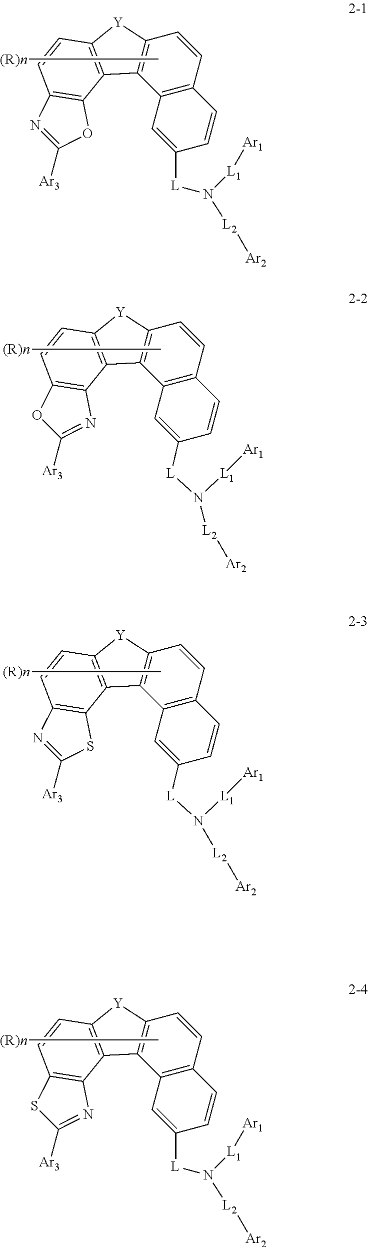

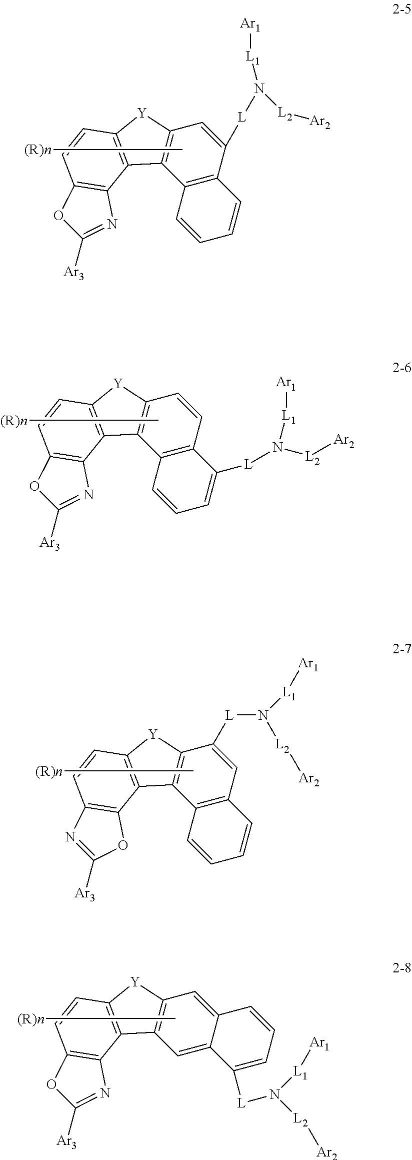

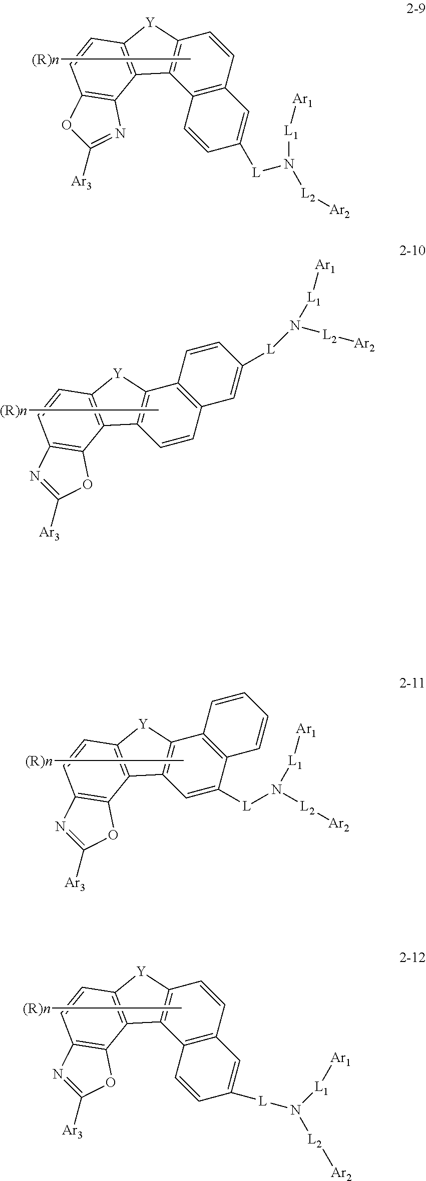

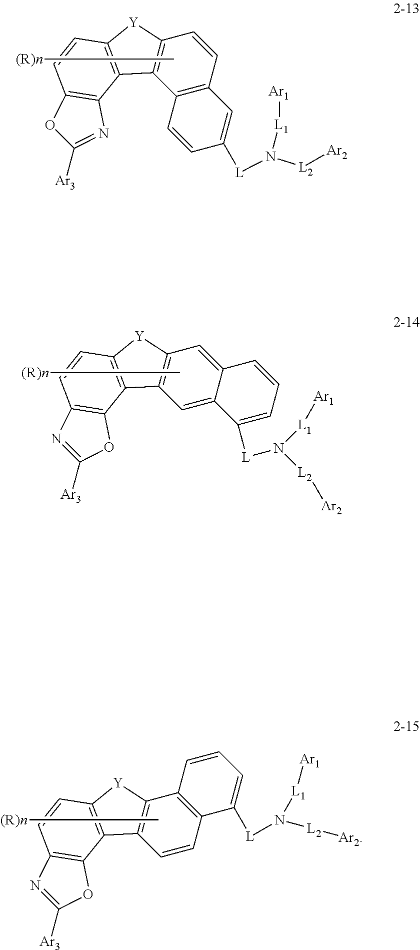

In some embodiments, Ar₁, Ar₂, and Ar₃ are the same or different, and are each independently selected from a substituted or unsubstituted aryl with 6 to 25 carbon atoms, or a substituted or unsubstituted heteroaryl with 5 to 24 carbon atoms.

In some embodiments, Ar₁, Ar₂, and Ars are each independently selected from a substituted or unsubstituted aryl with 6, 7, 8, 9, 10, 11, 12, 13, 14, 15, 16, 17, 18, 19, 20, 21, 22, 23, 24, or 25 carbon atoms, or a substituted or unsubstituted heteroaryl with 5, 6, 7, 8, 9, 10, 12, 13, 14, 15, 16, 17, 18, 19, 20, 21, 22, 23, or 24 carbon atoms.

In some embodiments, the substituents of Ar₁, Ar₂, and Ar₃ are each independently selected from deuterium, a halogen group, a cyano, a haloalkyl with 1 to 4 carbon atoms, a deuteroalkyl with 1 to 4 carbon atoms, an alkyl with 1 to 4 carbon atoms, a cycloalkyl with 5 to 10 carbon atoms, an aryl with 6 to 15 carbon atoms, a heteroaryl with 5 to 12 carbon atoms, a trialkylsilyl with 3 to 8 carbon atoms, or a deuteroaryl with 6 to 15 carbon atoms, and optionally, any two adjacent substituents form a benzene ring or a fluorene ring.

In some embodiments, Ar₁, Ar₂, and Ar₃ are each independently selected from a substituted or unsubstituted phenyl, a substituted or unsubstituted biphenyl, a substituted or unsubstituted terphenyl, a substituted or unsubstituted naphthyl, a substituted or unsubstituted anthracyl, a substituted or unsubstituted phenanthryl, a substituted or unsubstituted fluorenyl, a substituted or unsubstituted spirodifluorenyl, a substituted or unsubstituted triphenylene, a substituted or unsubstituted pyrenyl, a substituted or unsubstituted perylenyl, a substituted or unsubstituted dibenzothiophenyl, a substituted or unsubstituted dibenzofuranyl, a substituted or unsubstituted carbazolyl, a substituted or unsubstituted benzothiazolyl, a substituted or unsubstituted benzoxazolyl, or a substituted or unsubstituted benzimidazolyl.

Optionally, the substituents of Ar₁, Ar₂, and Ar₃ are each independently selected from deuterium, a fluorine, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a pyridyl, a dibenzofuranyl, a dibenzothiophenyl, or a carbazolyl; and optionally, in Ar₁ and Ar₂, any two adjacent substituents form a benzene ring.

In some embodiments, Ar₁, Ar₂, and Ar₃ are each independently selected from a substituted or unsubstituted group W, where the unsubstituted group W is selected from the group consisting of the following groups:

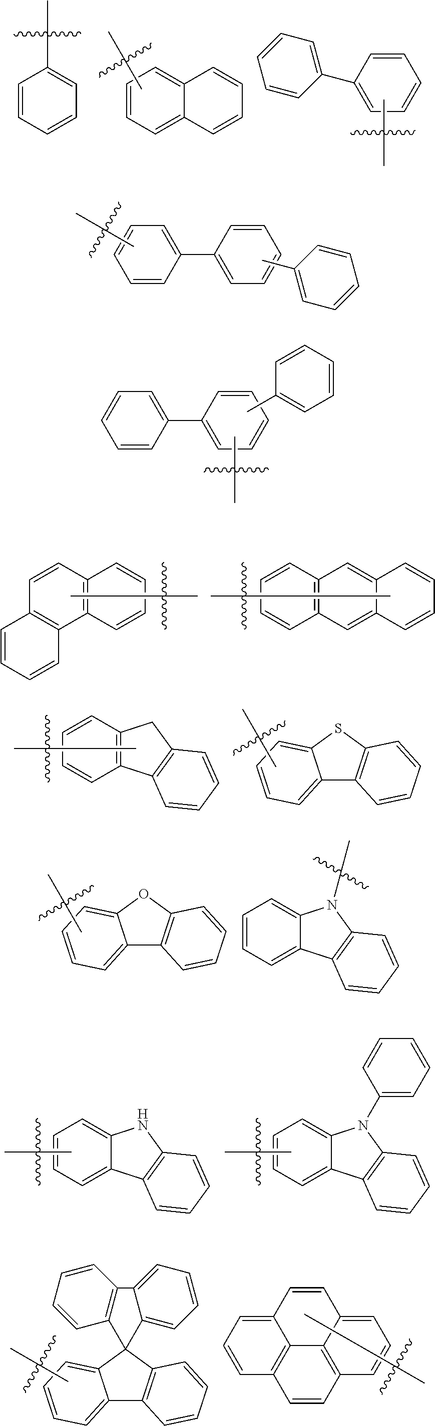

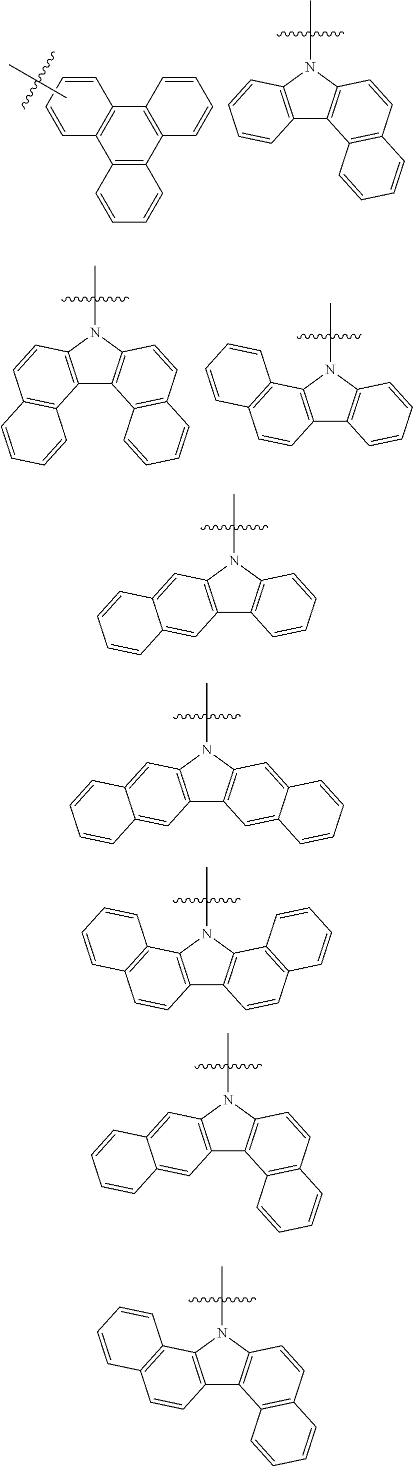

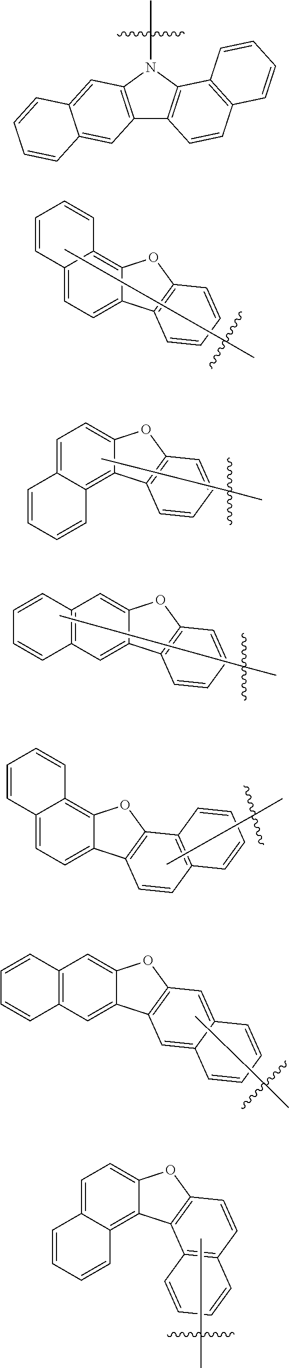

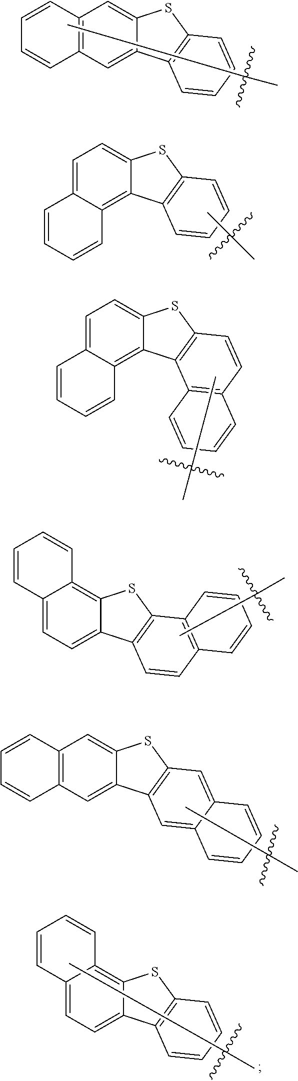

and

the substituted group W has one or more substituents, and the substituents of the substituted group W are each independently selected from deuterium, a fluorine, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a dibenzofuranyl, a dibenzothiophenyl, or a carbazolyl, and when the number of substituents of the group W is greater than 1, each substituent is the same or different.

In some embodiments, Ar₁ and Ar₂ are each independently selected from the group consisting of the following groups:

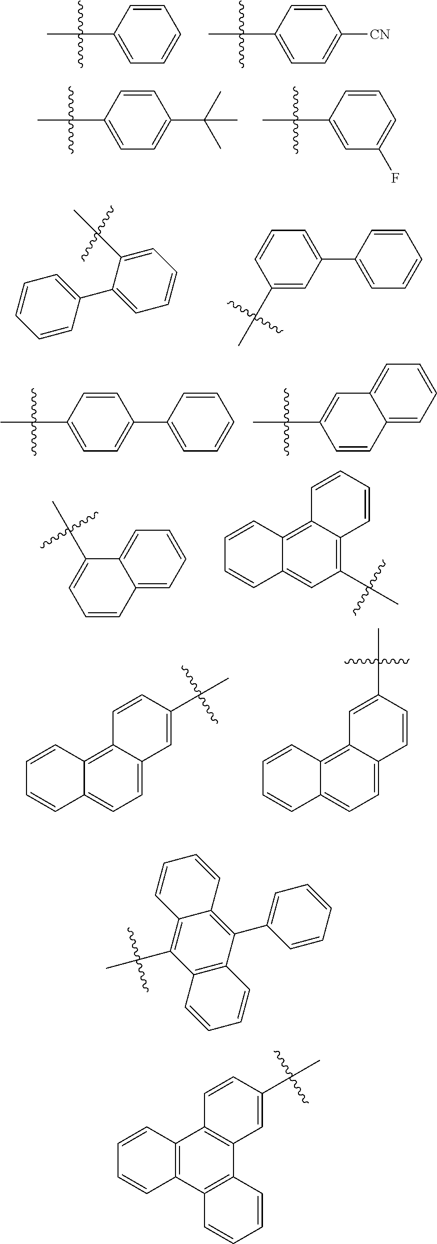

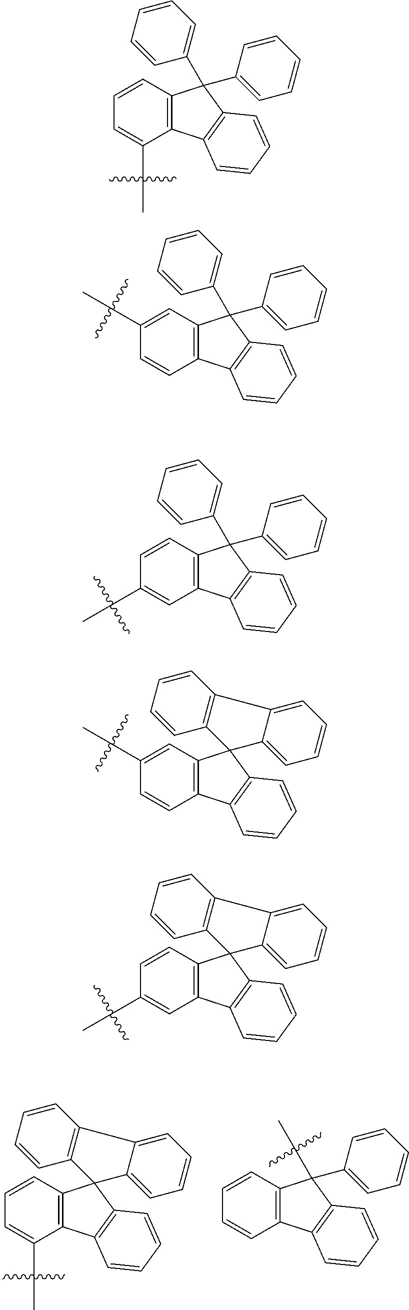

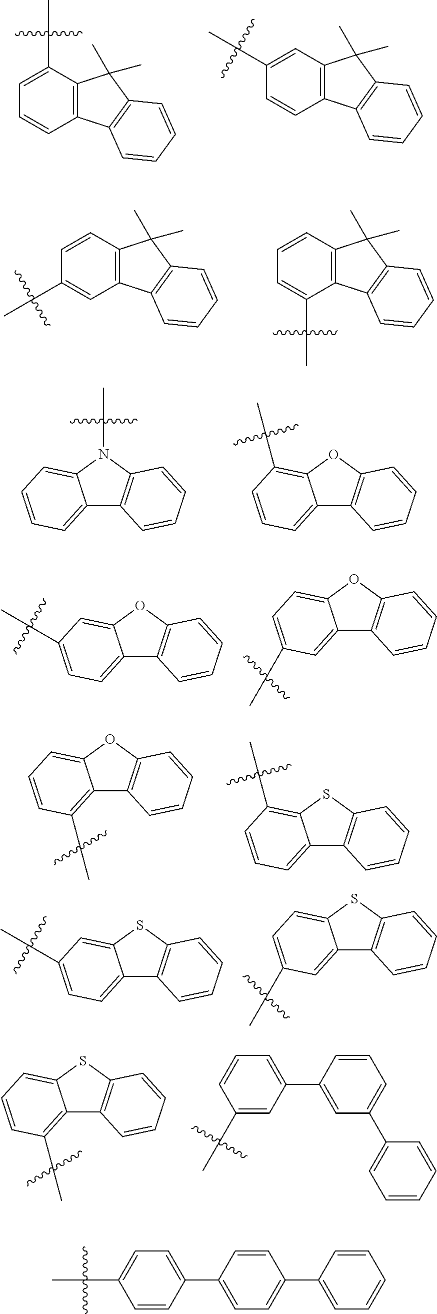

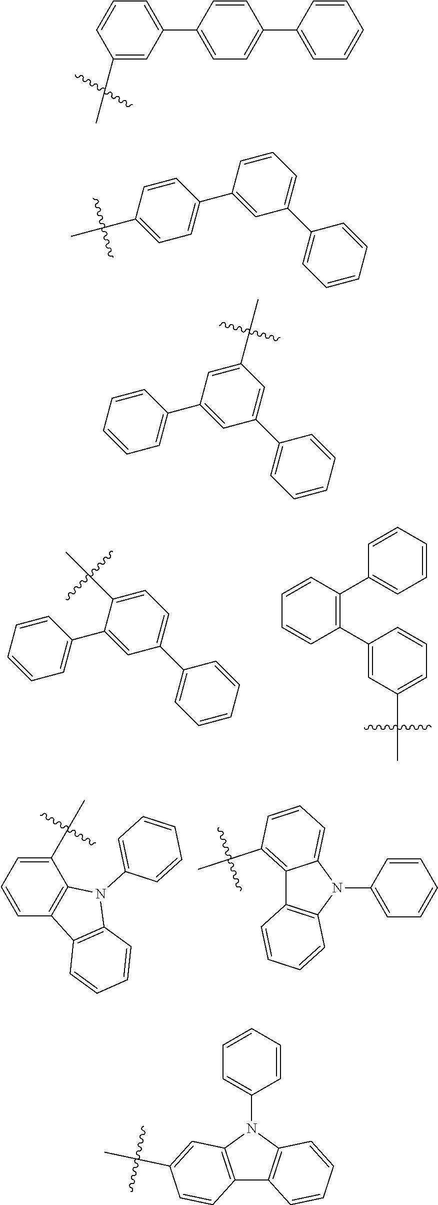

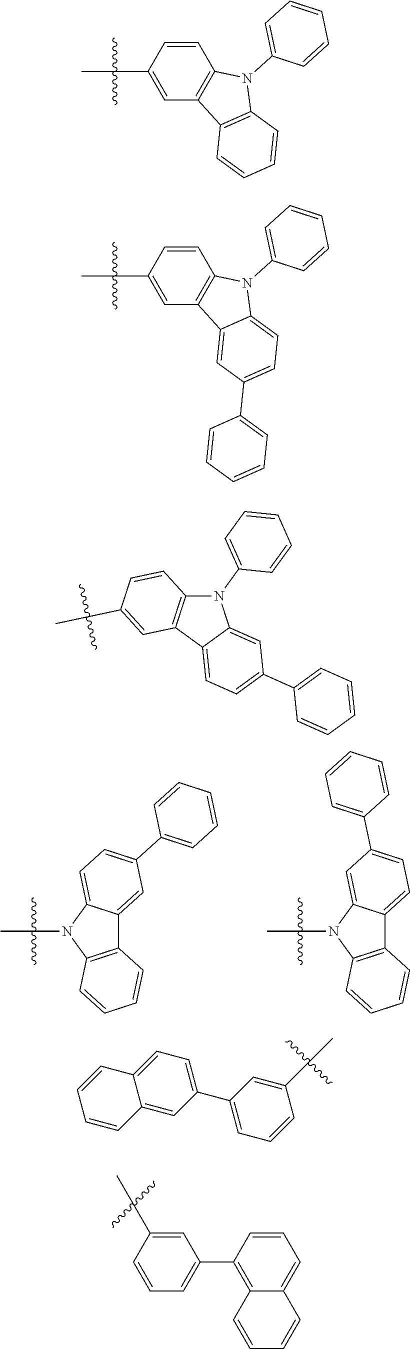

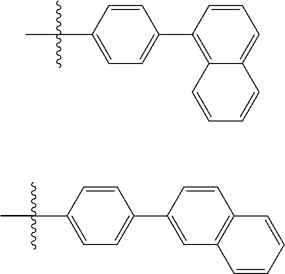 [SMILES: *c1ccc(-c2cccc3ccccc23)cc1 ; *c1ccc(-c2ccc3ccccc3c2)cc1]

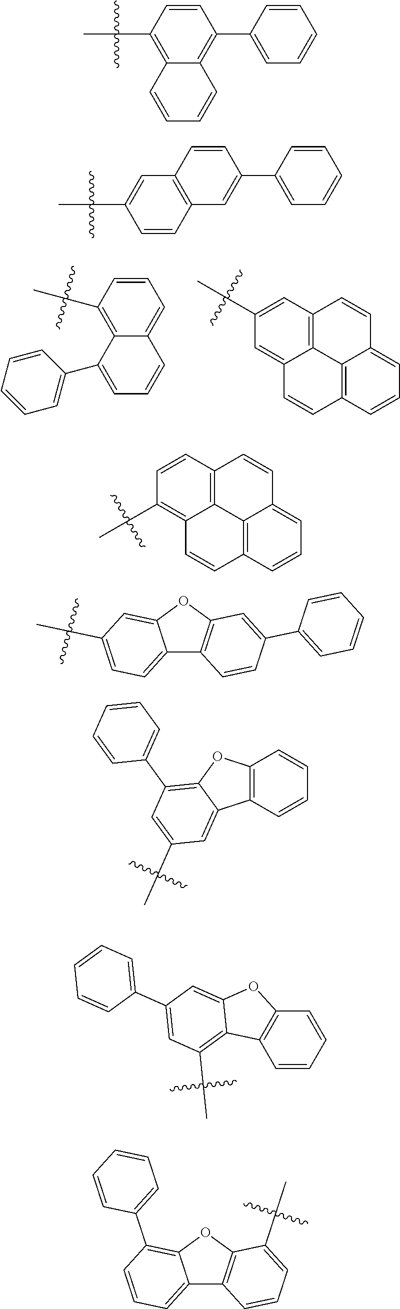

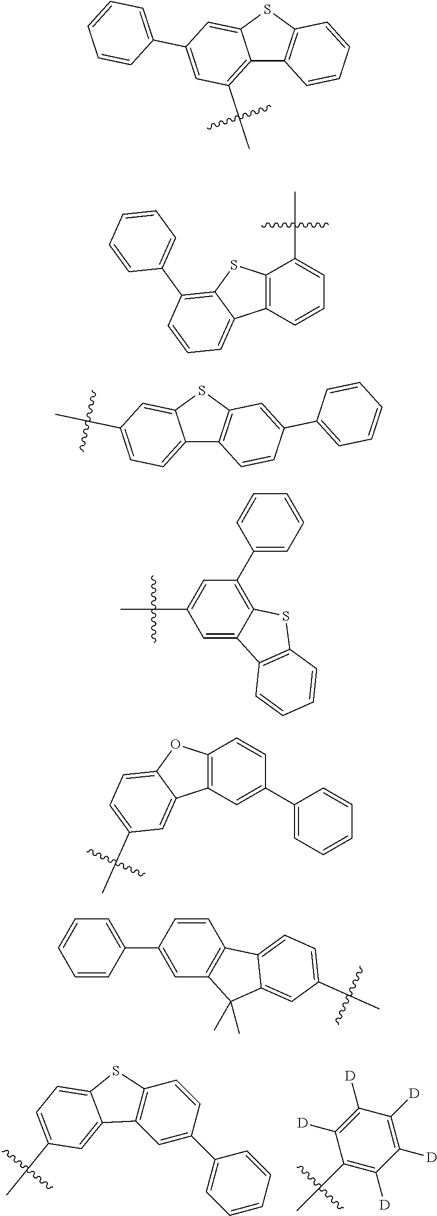

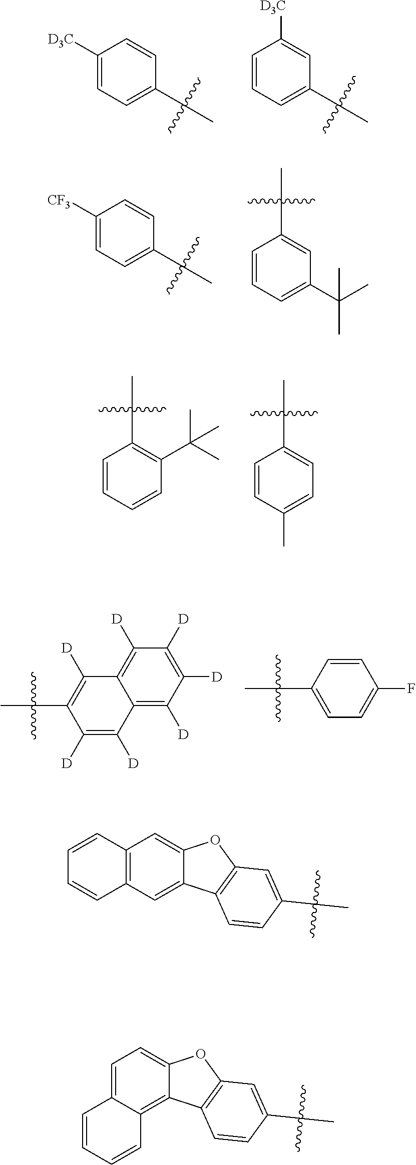

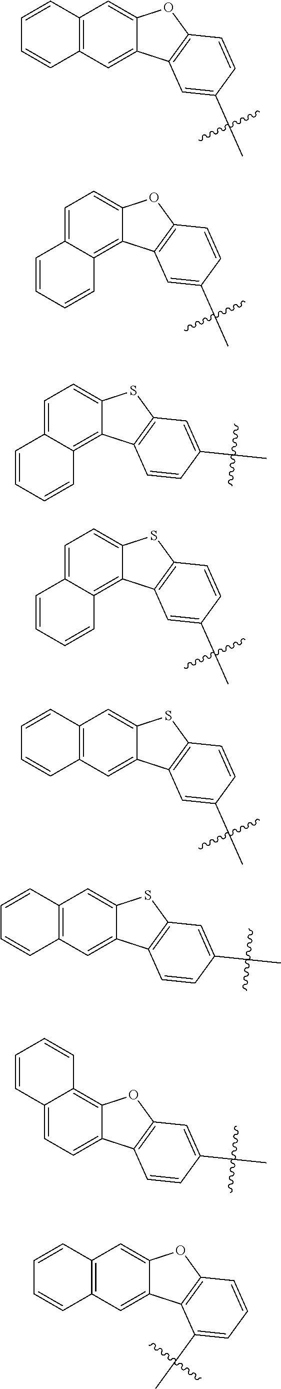

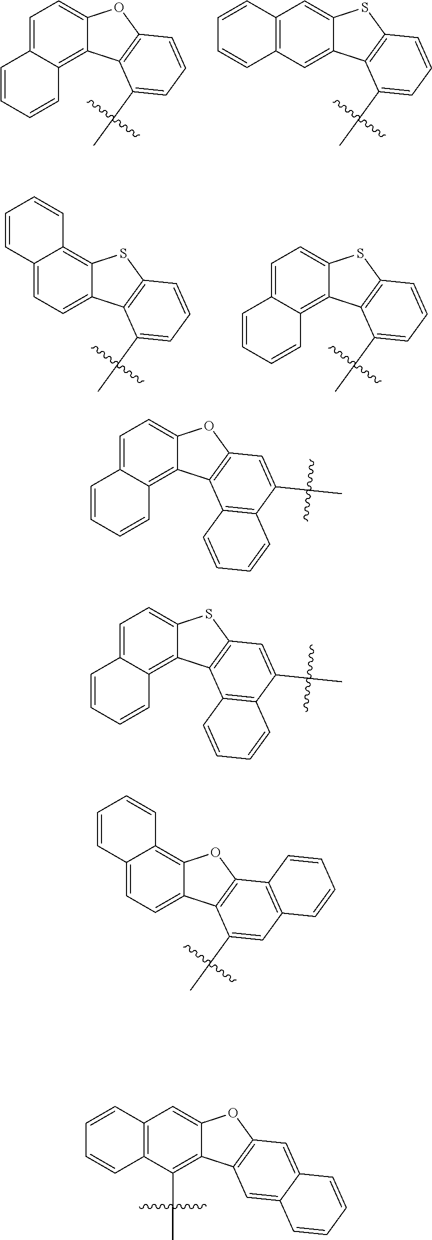

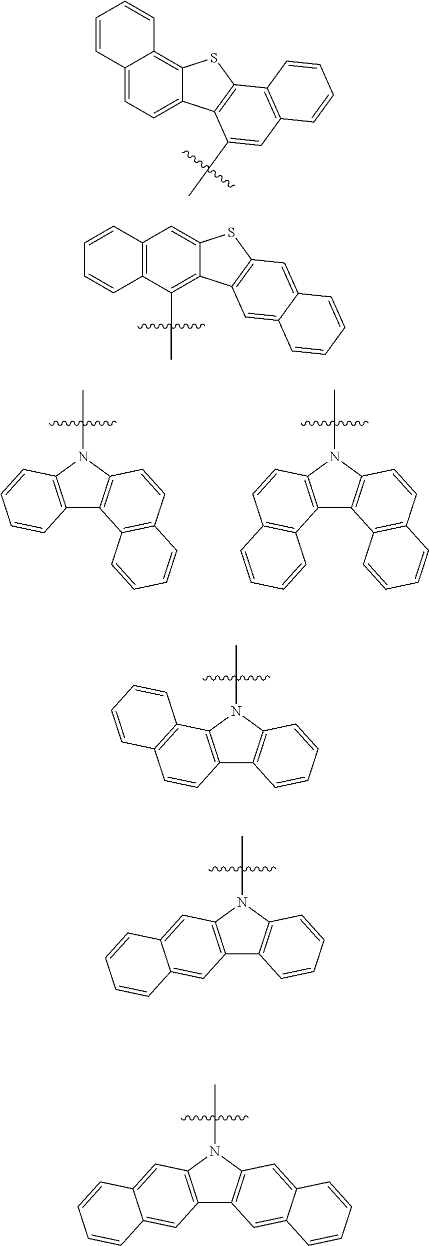

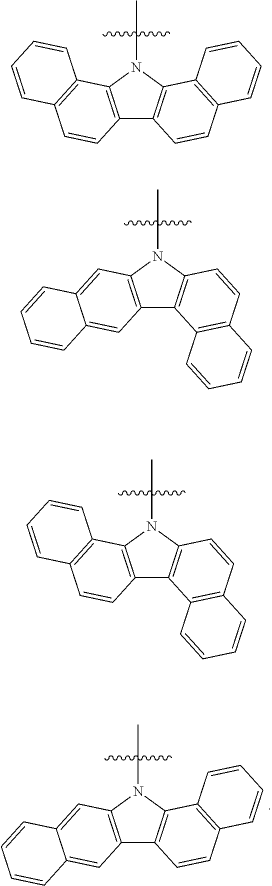 [SMILES: *n1c2c3ccccc3ccc2c2ccc3ccccc3c21 ; *n1c2cc3ccccc3cc2c2c3ccccc3ccc21 ; *n1c2ccc3ccccc3c2c2ccc3ccccc3c21 ; *n1c2cc3ccccc3cc2c2ccc3ccccc3c21]

In some embodiments, Ar₃ is selected from a substituted or unsubstituted aryl with 6 to 18 carbon atoms, or a substituted or unsubstituted heteroaryl with 12 to 18 carbon atoms; and the substituents of Ar₃ are independently selected from deuterium, a fluorine, a cyano, a trideuteromethyl, a trimethylsilyl, a trifluoromethyl, a cyclopentyl, a cyclohexyl, an adamantalkyl, a methyl, an ethyl, an isopropyl, a tert-butyl, a phenyl, a naphthyl, a pyridyl, or a deuterophenyl.

In some embodiments, Ar₃ is selected from the group consisting of the following groups:

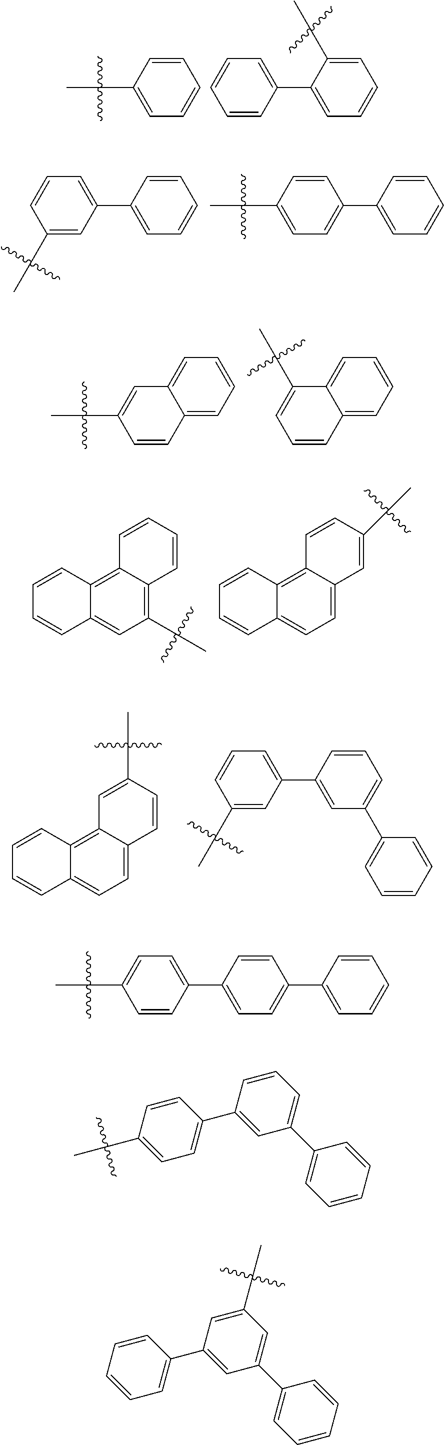

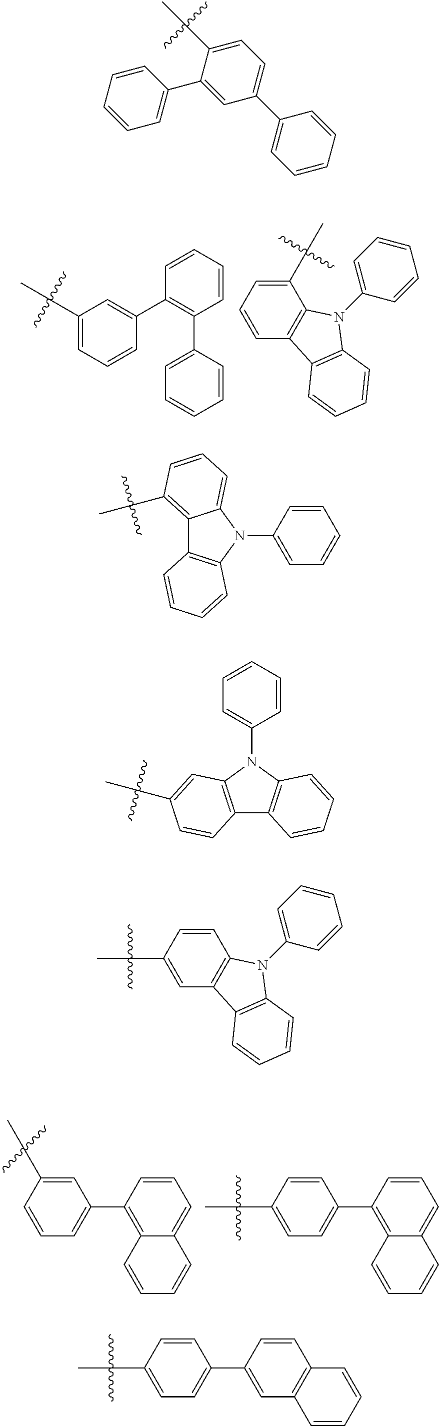

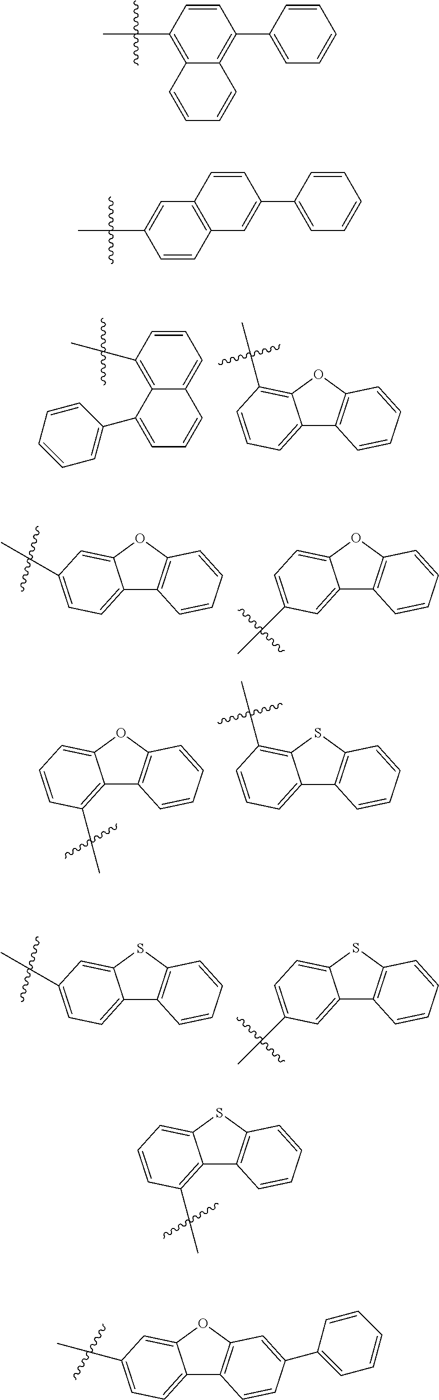

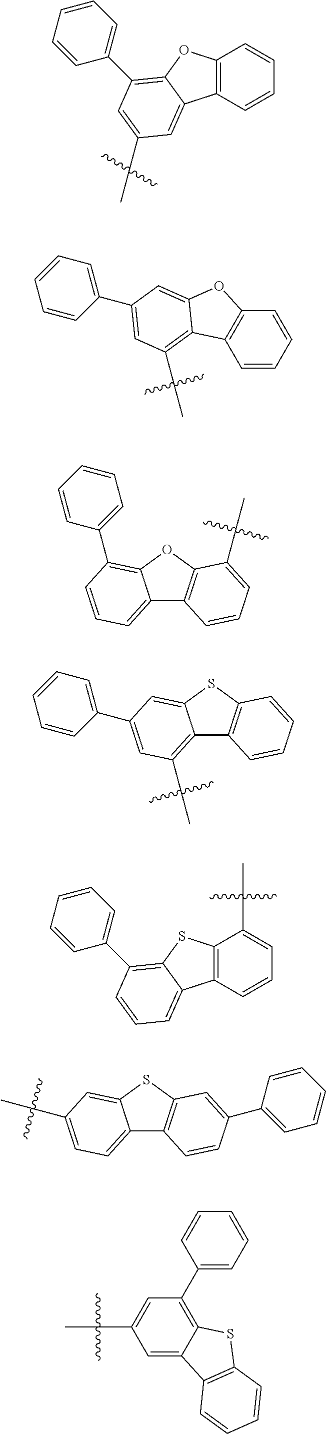

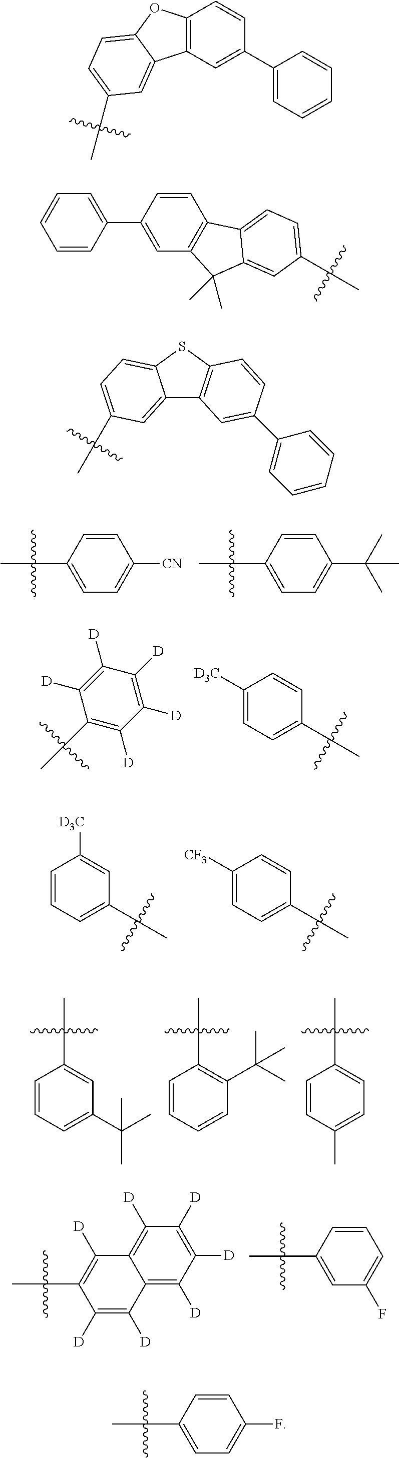

In some embodiments, L, L₁, and L₂ are the same or different, and are each independently selected from a single bond, a substituted or unsubstituted arylene with 6 to 18 carbon atoms, or a substituted or unsubstituted heteroarylene with 5 to 18 carbon atoms.

In some embodiments, L, L₁, and L₂ are the same or different, and are each independently selected from a single bond; a substituted or unsubstituted arylene with 6, 7, 8, 9, 10, 11, 12, 13, 14, or 15 carbon atoms; or a substituted or unsubstituted heteroarylene with 5, 6, 7, 8, 9, 10, 11, 12, 13, 14, 15, 16, 17, or 18 carbon atoms.

Optionally, the substituents of L, L₁, and L₂ are each independently selected from deuterium, a fluorine, a cyano, an alkyl with 1 to 5 carbon atoms, a trialkylsilyl with 3 to 8 carbon atoms, a fluoroalkyl with 1 to 4 carbon atoms, a deuteratoalkyl with 1 to 4 carbon atoms, a phenyl, or a naphthyl.

In some embodiments, L, L₁, and L₂ are each independently selected from a single bond, a substituted or unsubstituted phenylene, a substituted or unsubstituted naphthylene, a substituted or unsubstituted biphenylene, a substituted or unsubstituted fluorenylene, a substituted or unsubstituted phenanthrylene, a substituted or unsubstituted dibenzothiophenylene, a substituted or unsubstituted dibenzofuranylene, or a substituted or unsubstituted carbazolylene.

Optionally, the substituents of L, L₁, and L₂ are the same or different, and are each independently selected from deuterium, a fluorine, a cyano, a methyl, an ethyl, an isopropyl, a tert-butyl, a trifluoromethyl, a trideuteromethyl, a trimethylsilyl, or a phenyl.

In some embodiments, L, L₁, and L₂ are each independently selected from a single bond or a substituted or unsubstituted group Q, and the unsubstituted group Q is selected from the group consisting of the following groups:

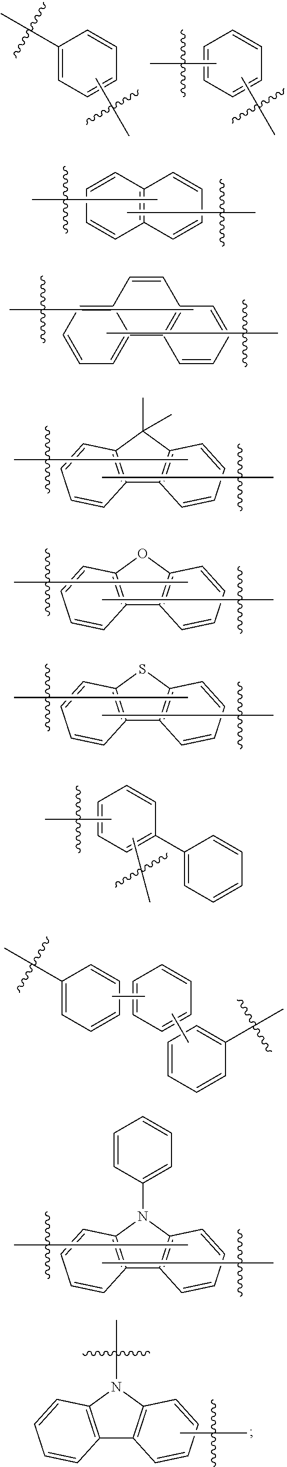

and

the substituted group Q has one or more substituents, and the substituents of the substituted group Q are independently selected from deuterium, a fluorine, a cyano, a trideuteromethyl, a trimethylsilyl, trifluoromethyl, a cyclopentyl, a cyclohexyl, an adamantyl, a methyl, an ethyl, an isopropyl, a tert-butyl, a phenyl, a naphthyl, a dibenzofuranyl, a dibenzothiophenyl, or a carbazolyl, and when the number of substituents of the group Q is greater than 1, each substituent is the same or different.

In some embodiments, Ars is selected from the group consisting of the following groups:

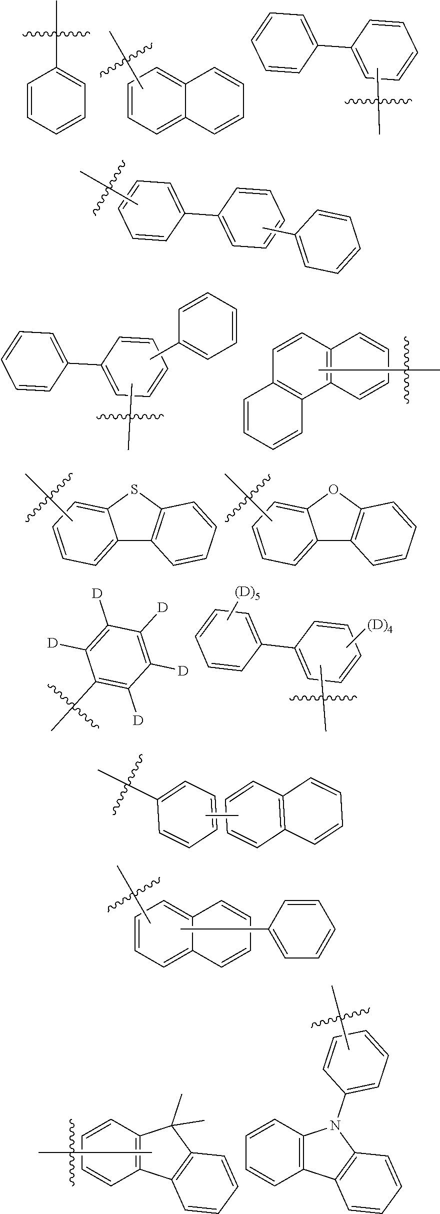

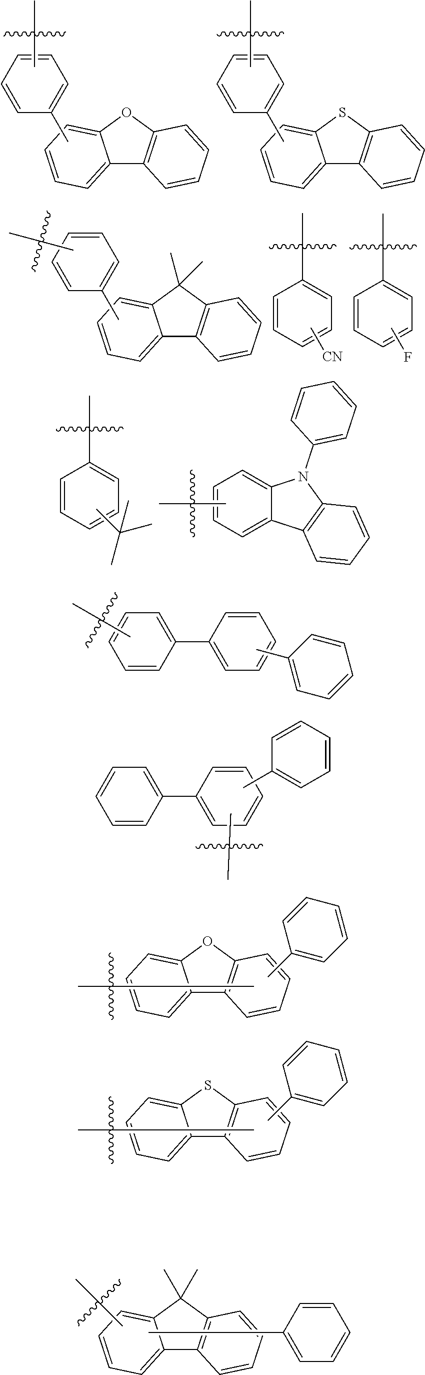

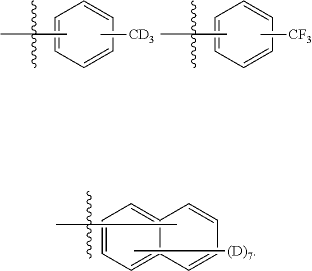

In some embodiments,

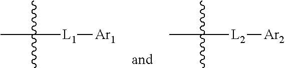

are each independently selected from the group consisting of the following groups:

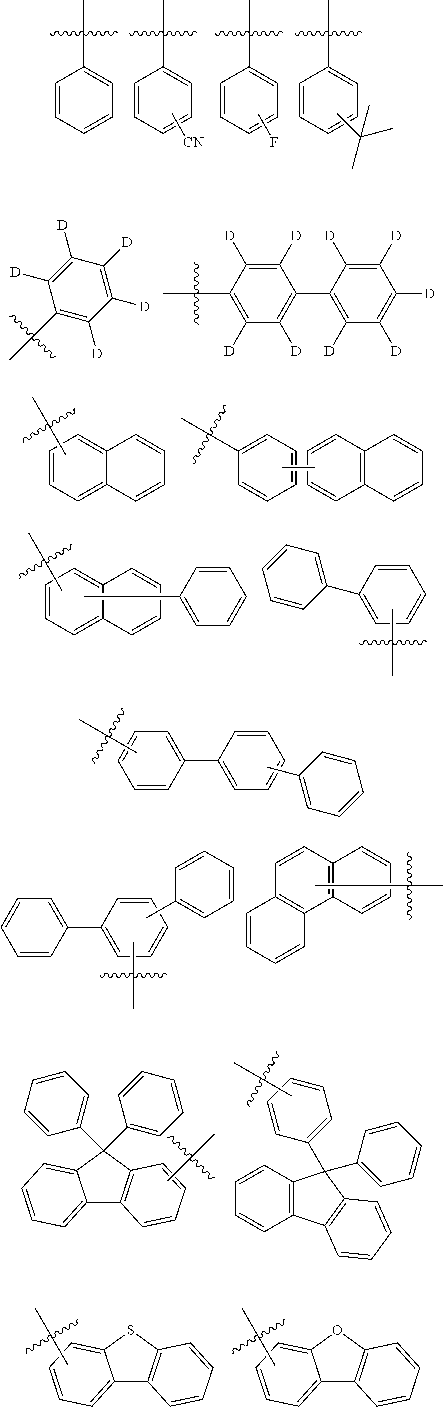

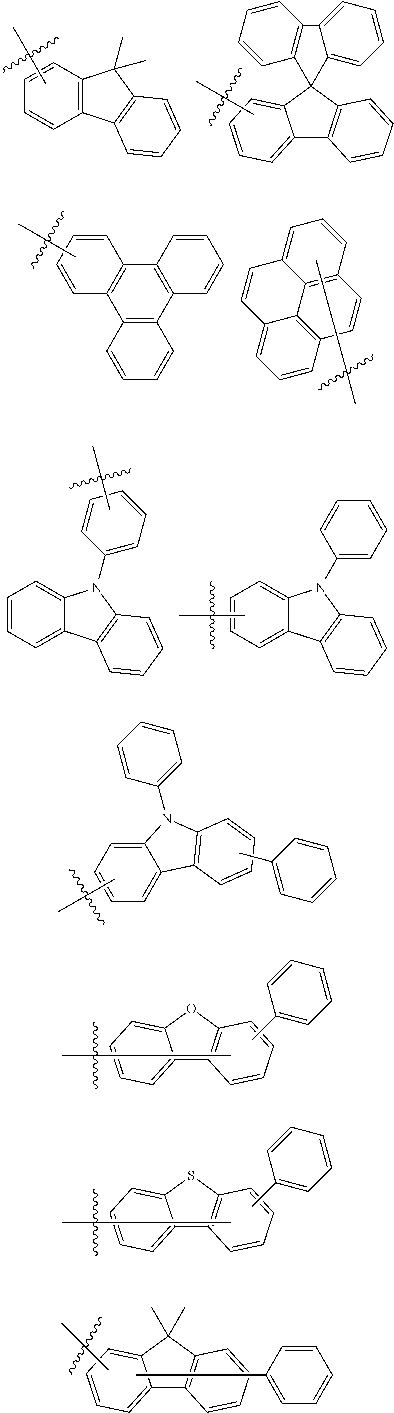

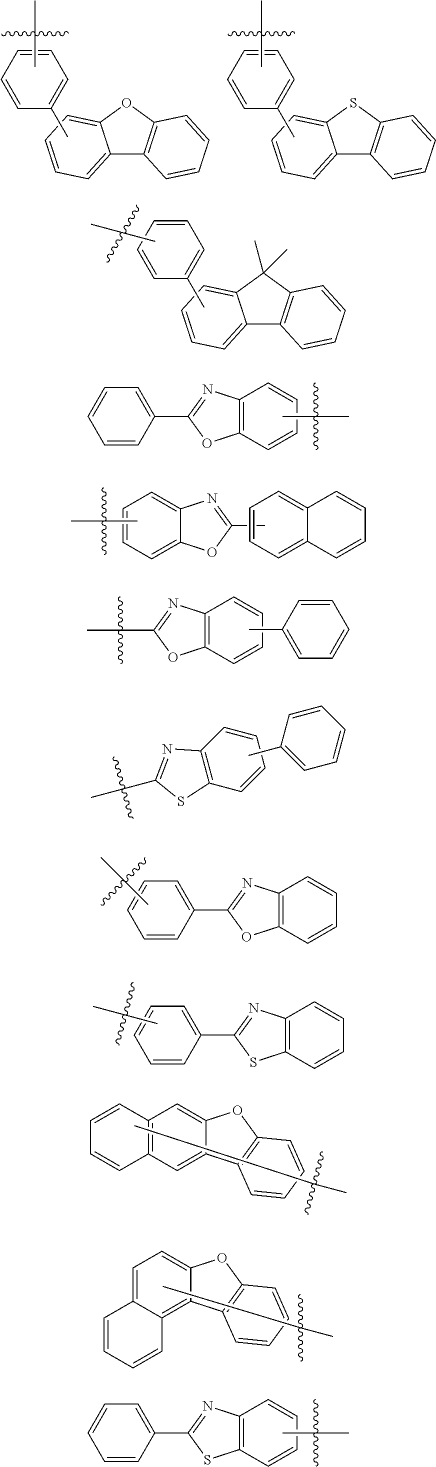

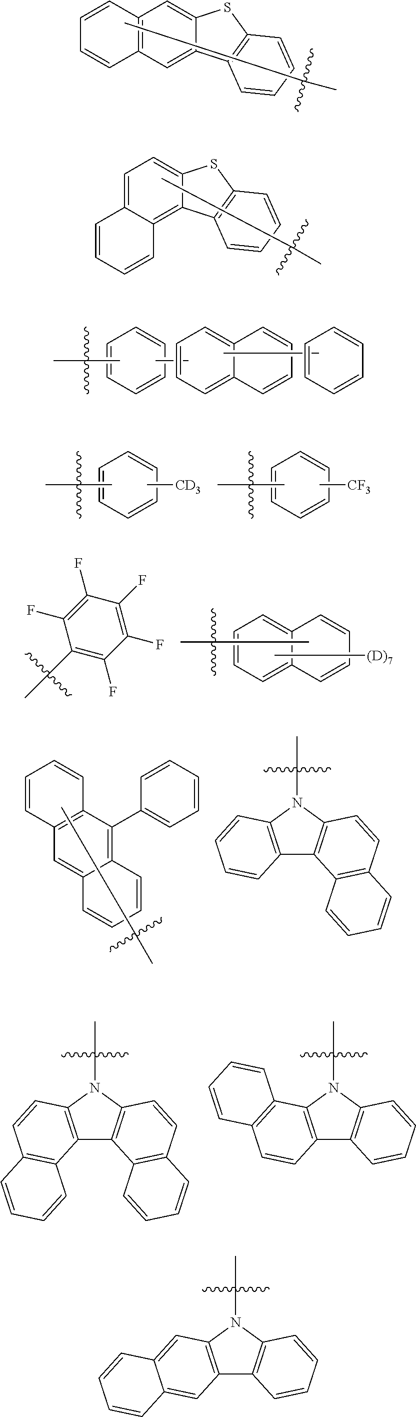

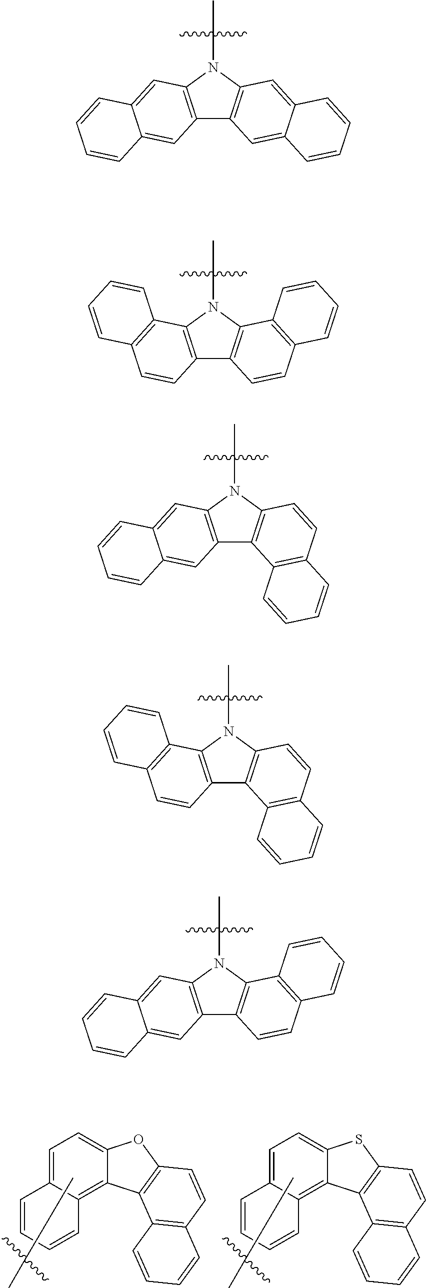

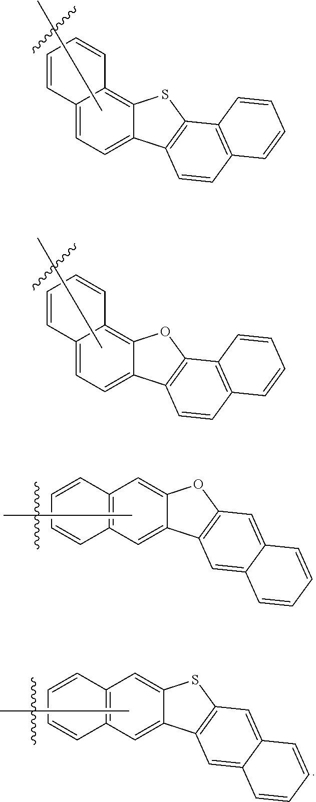

In some embodiments, L is selected from a single bond or the group consisting of the following groups:

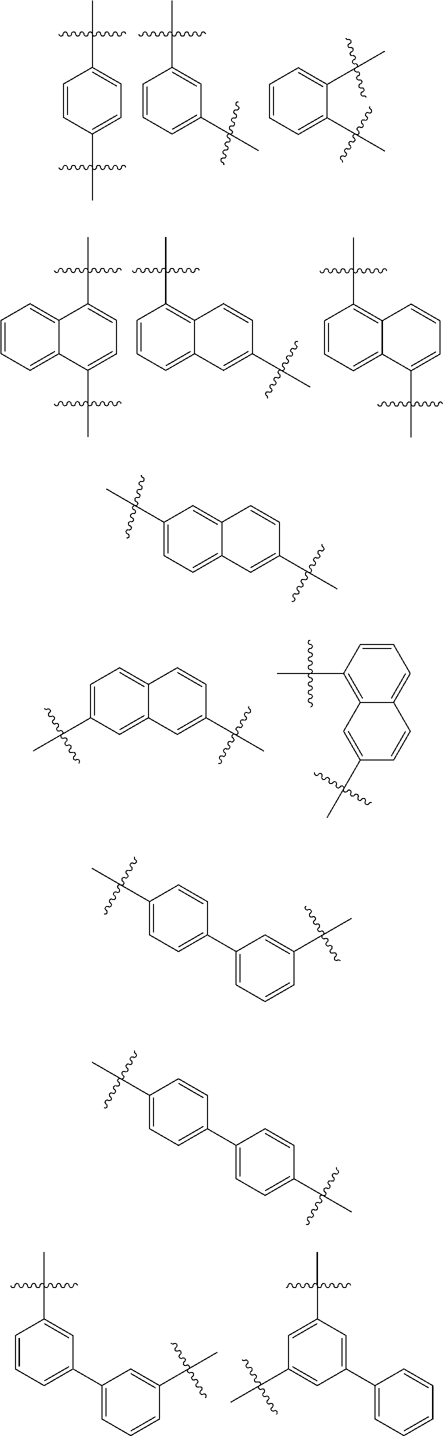

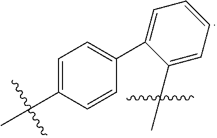 [SMILES: *c1ccc(-c2ccccc2*)cc1]

In some embodiments, L₁ and L₂ are each independently selected from a single bond or the group consisting of the following groups:

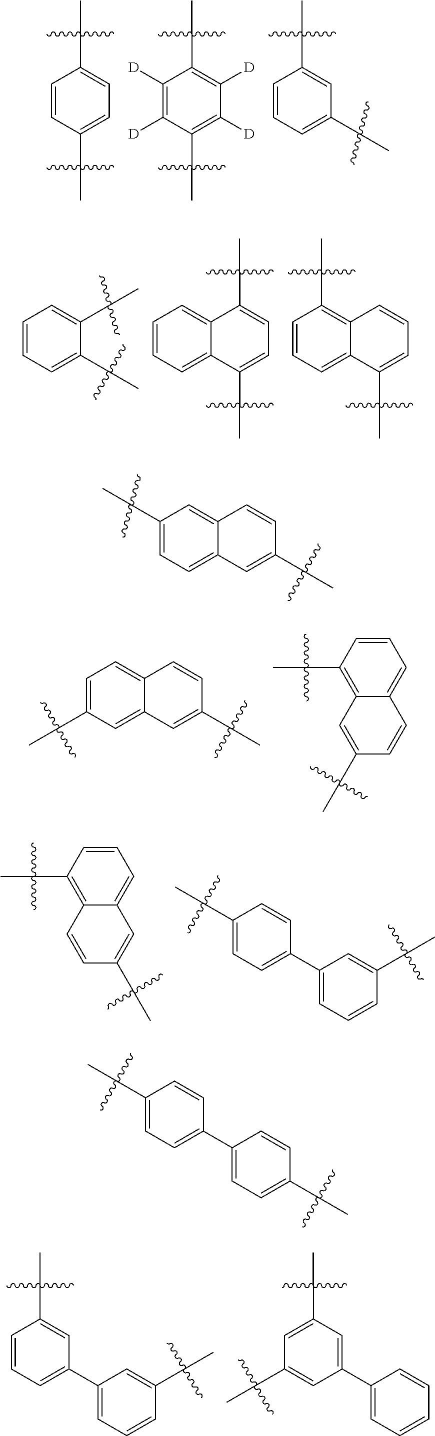

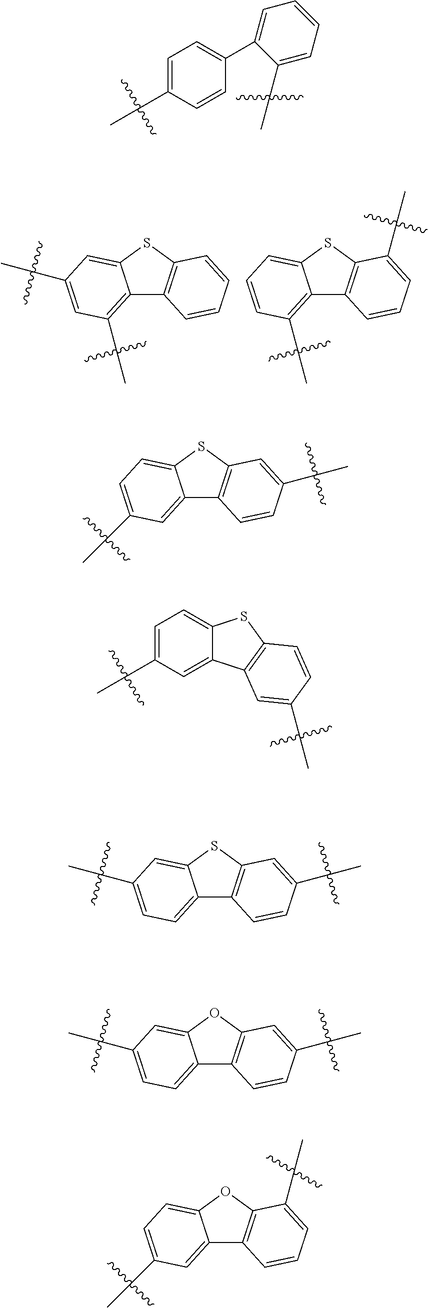

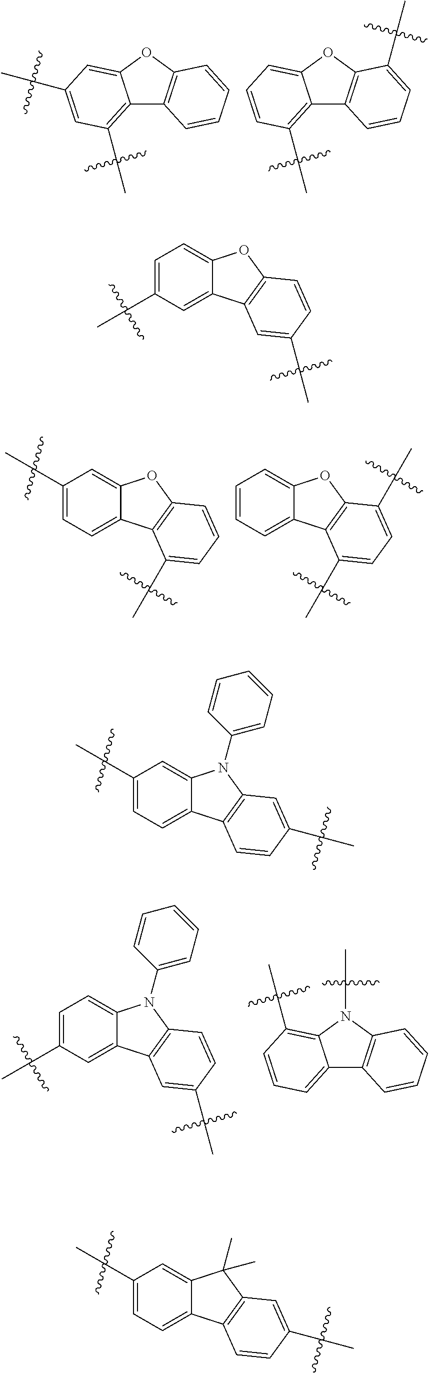

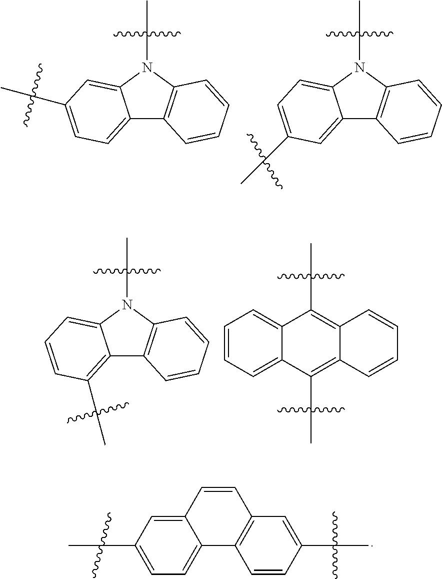

In some embodiments, each R is the same or different, and is each independently selected from deuterium, a cyano, a fluorine, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a dibenzofuranyl, a dibenzothiophenyl, or a carbazoyl; and optionally, any two adjacent substituents form a benzene ring.

In some embodiments, the heterocyclic compound is selected from the group consisting of the following compounds:

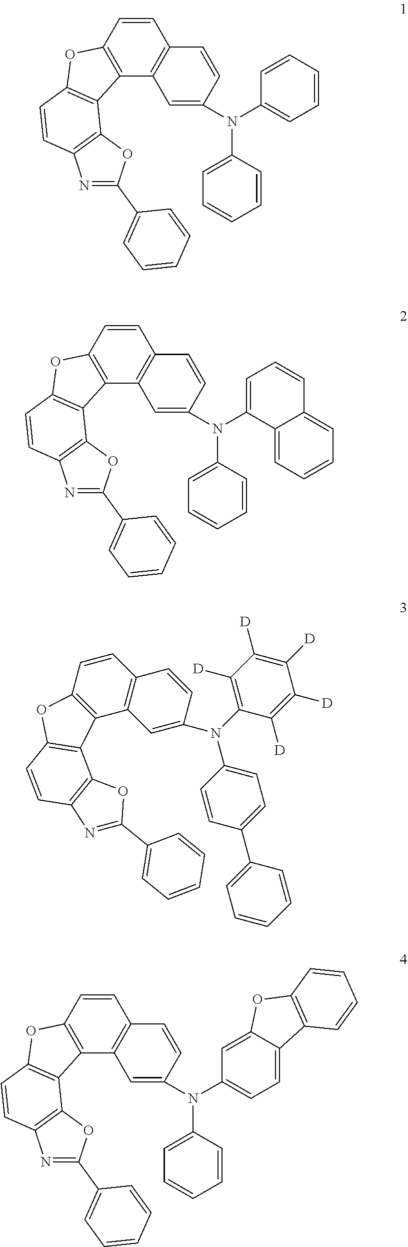 [SMILES: c1ccc(-c2nc3ccc4oc5ccc6ccc(N(c7ccccc7)c7ccccc7)cc6c5c4c3o2)cc1 ; c1ccc(-c2nc3ccc4oc5ccc6ccc(N(c7ccccc7)c7cccc8ccccc78)cc6c5c4c3o2)cc1 ; [2H]c1c([2H])c([2H])c(N(c2ccc(-c3ccccc3)cc2)c2ccc3ccc4oc5ccc6nc(-c7ccccc7)oc6c5c4c3c2)c([2H])c1[2H] ; c1ccc(-c2nc3ccc4oc5ccc6ccc(N(c7ccccc7)c7ccc8c(c7)oc7ccccc78)cc6c5c4c3o2)cc1]

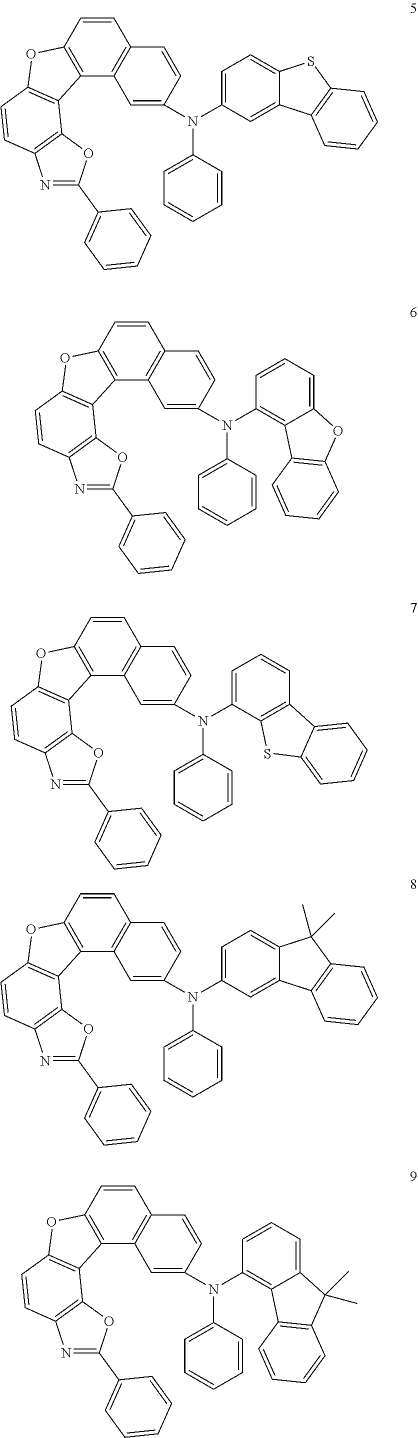

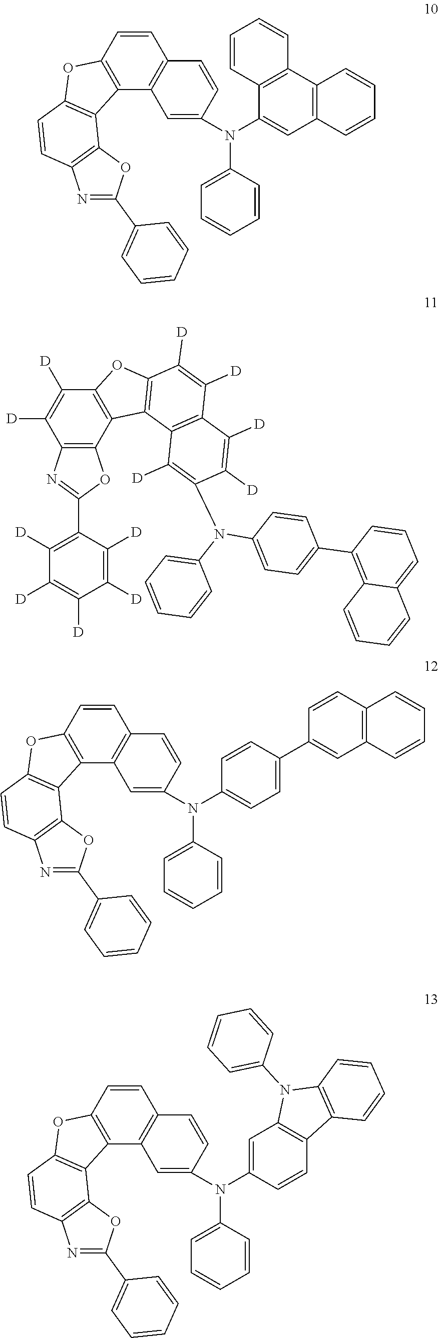 [SMILES: c1ccc(-c2nc3ccc4oc5ccc6ccc(N(c7ccccc7)c7cc8ccccc8c8ccccc78)cc6c5c4c3o2)cc1 ; [2H]c1c([2H])c([2H])c(-c2nc3c([2H])c([2H])c4oc5c([2H])c([2H])c6c([2H])c([2H])c(N(c7ccccc7)c7ccc(-c8cccc9ccccc89)cc7)c([2H])c6c5c4c3o2)c([2H])c1[2H] ; c1ccc(-c2nc3ccc4oc5ccc6ccc(N(c7ccccc7)c7ccc(-c8ccc9ccccc9c8)cc7)cc6c5c4c3o2)cc1 ; c1ccc(-c2nc3ccc4oc5ccc6ccc(N(c7ccccc7)c7ccc8c9ccccc9n(-c9ccccc9)c8c7)cc6c5c4c3o2)cc1]

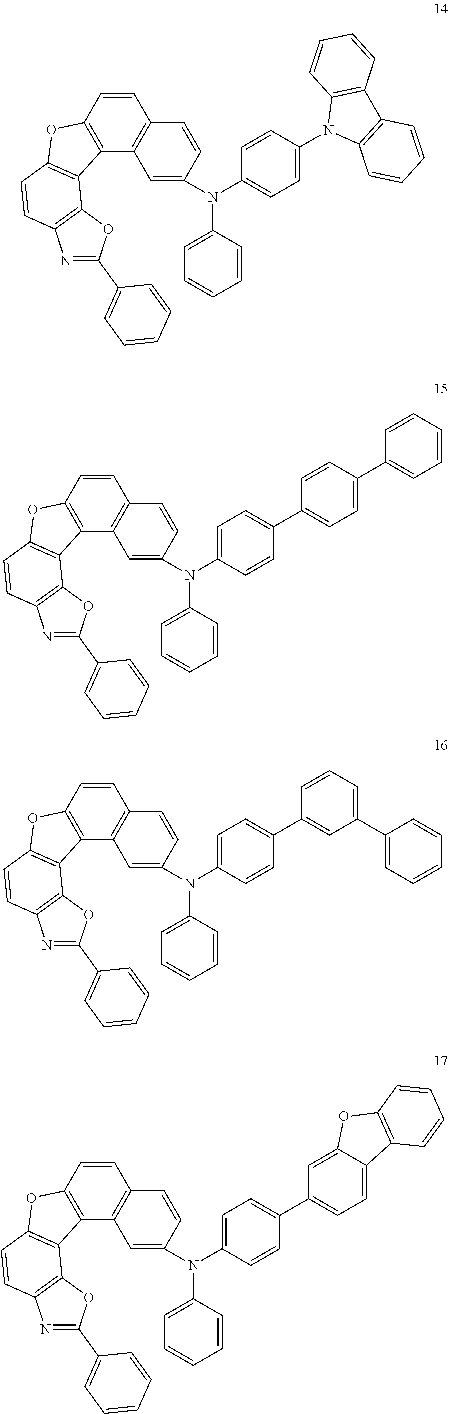 [SMILES: c1ccc(-c2nc3ccc4oc5ccc6ccc(N(c7ccccc7)c7ccc(-n8c9ccccc9c9ccccc98)cc7)cc6c5c4c3o2)cc1 ; c1ccc(-c2ccc(-c3ccc(N(c4ccccc4)c4ccc5ccc6oc7ccc8nc(-c9ccccc9)oc8c7c6c5c4)cc3)cc2)cc1 ; c1ccc(-c2cccc(-c3ccc(N(c4ccccc4)c4ccc5ccc6oc7ccc8nc(-c9ccccc9)oc8c7c6c5c4)cc3)c2)cc1 ; c1ccc(-c2nc3ccc4oc5ccc6ccc(N(c7ccccc7)c7ccc(-c8ccc9c(c8)oc8ccccc89)cc7)cc6c5c4c3o2)cc1]

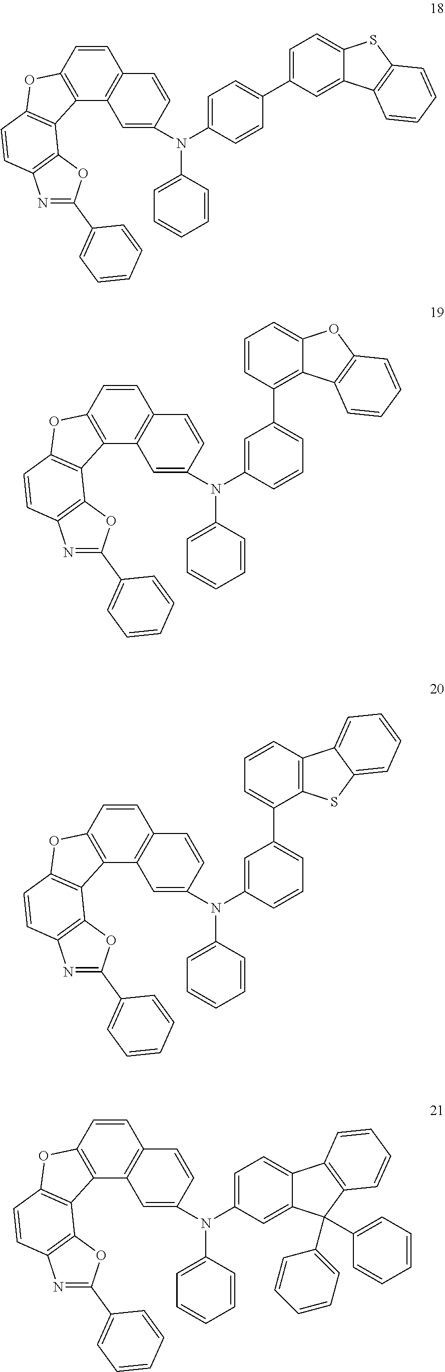 [SMILES: c1ccc(-c2nc3ccc4oc5ccc6ccc(N(c7ccccc7)c7ccc(-c8ccc9sc%10ccccc%10c9c8)cc7)cc6c5c4c3o2)cc1 ; c1ccc(-c2nc3ccc4oc5ccc6ccc(N(c7ccccc7)c7cccc(-c8cccc9oc%10ccccc%10c89)c7)cc6c5c4c3o2)cc1 ; c1ccc(-c2nc3ccc4oc5ccc6ccc(N(c7ccccc7)c7cccc(-c8cccc9c8sc8ccccc89)c7)cc6c5c4c3o2)cc1 ; c1ccc(-c2nc3ccc4oc5ccc6ccc(N(c7ccccc7)c7ccc8c(c7)C(c7ccccc7)(c7ccccc7)c7ccccc7-8)cc6c5c4c3o2)cc1]

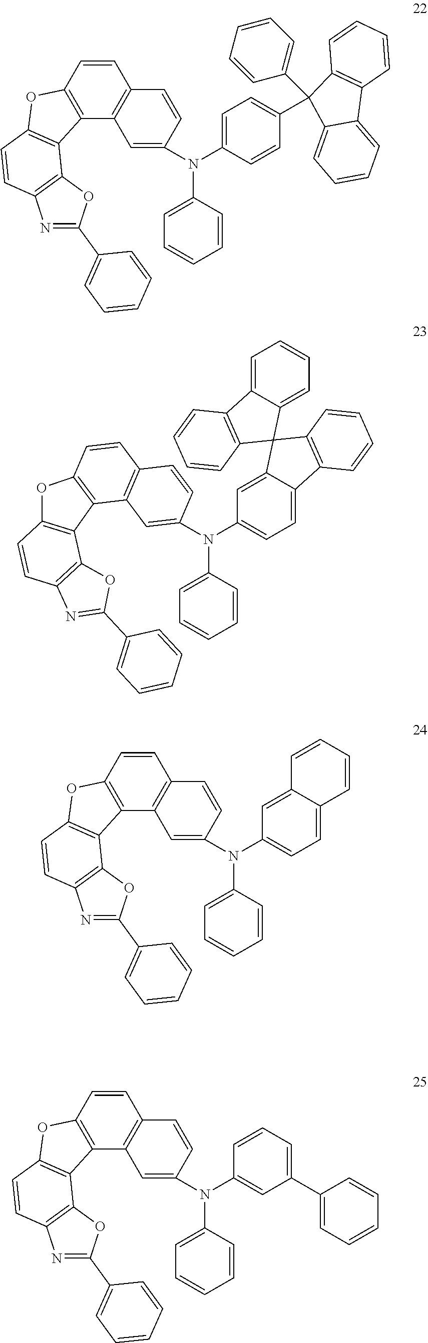 [SMILES: c1ccc(-c2nc3ccc4oc5ccc6ccc(N(c7ccccc7)c7ccc(C8(c9ccccc9)c9ccccc9-c9ccccc98)cc7)cc6c5c4c3o2)cc1 ; c1ccc(-c2nc3ccc4oc5ccc6ccc(N(c7ccccc7)c7ccc8c(c7)C7(c9ccccc9-c9ccccc97)c7ccccc7-8)cc6c5c4c3o2)cc1 ; c1ccc(-c2nc3ccc4oc5ccc6ccc(N(c7ccccc7)c7ccc8ccccc8c7)cc6c5c4c3o2)cc1 ; c1ccc(-c2cccc(N(c3ccccc3)c3ccc4ccc5oc6ccc7nc(-c8ccccc8)oc7c6c5c4c3)c2)cc1]

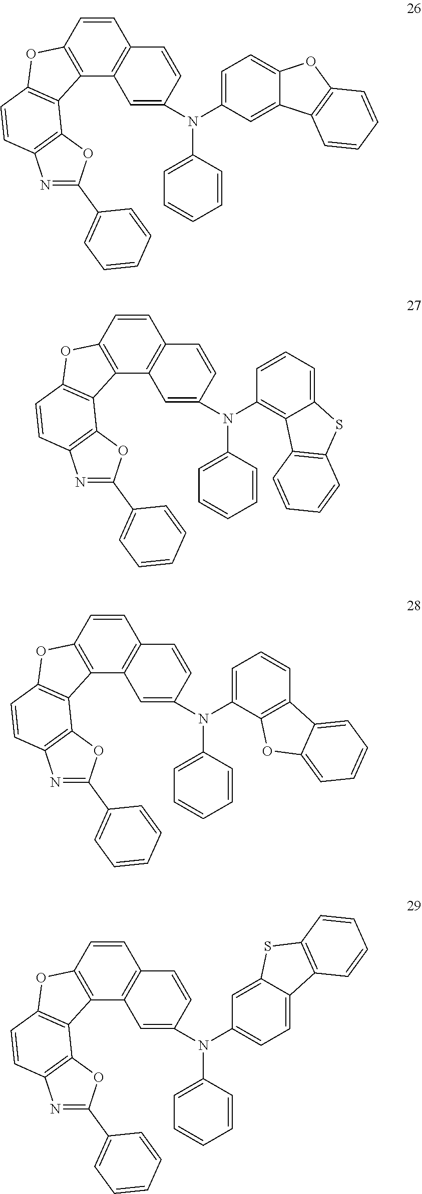 [SMILES: c1ccc(-c2nc3ccc4oc5ccc6ccc(N(c7ccccc7)c7ccc8oc9ccccc9c8c7)cc6c5c4c3o2)cc1 ; c1ccc(-c2nc3ccc4oc5ccc6ccc(N(c7ccccc7)c7cccc8sc9ccccc9c78)cc6c5c4c3o2)cc1 ; c1ccc(-c2nc3ccc4oc5ccc6ccc(N(c7ccccc7)c7cccc8c7oc7ccccc78)cc6c5c4c3o2)cc1 ; c1ccc(-c2nc3ccc4oc5ccc6ccc(N(c7ccccc7)c7ccc8c(c7)sc7ccccc78)cc6c5c4c3o2)cc1]

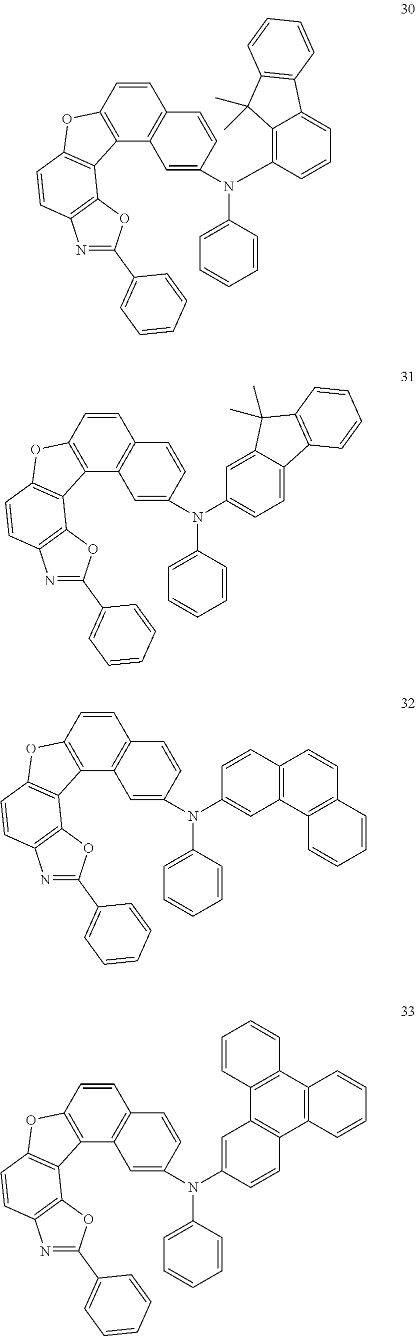 [SMILES: CC1(C)c2ccccc2-c2cccc(N(c3ccccc3)c3ccc4ccc5oc6ccc7nc(-c8ccccc8)oc7c6c5c4c3)c21 ; CC1(C)c2ccccc2-c2ccc(N(c3ccccc3)c3ccc4ccc5oc6ccc7nc(-c8ccccc8)oc7c6c5c4c3)cc21 ; c1ccc(-c2nc3ccc4oc5ccc6ccc(N(c7ccccc7)c7ccc8ccc9ccccc9c8c7)cc6c5c4c3o2)cc1 ; c1ccc(-c2nc3ccc4oc5ccc6ccc(N(c7ccccc7)c7ccc8c9ccccc9c9ccccc9c8c7)cc6c5c4c3o2)cc1]

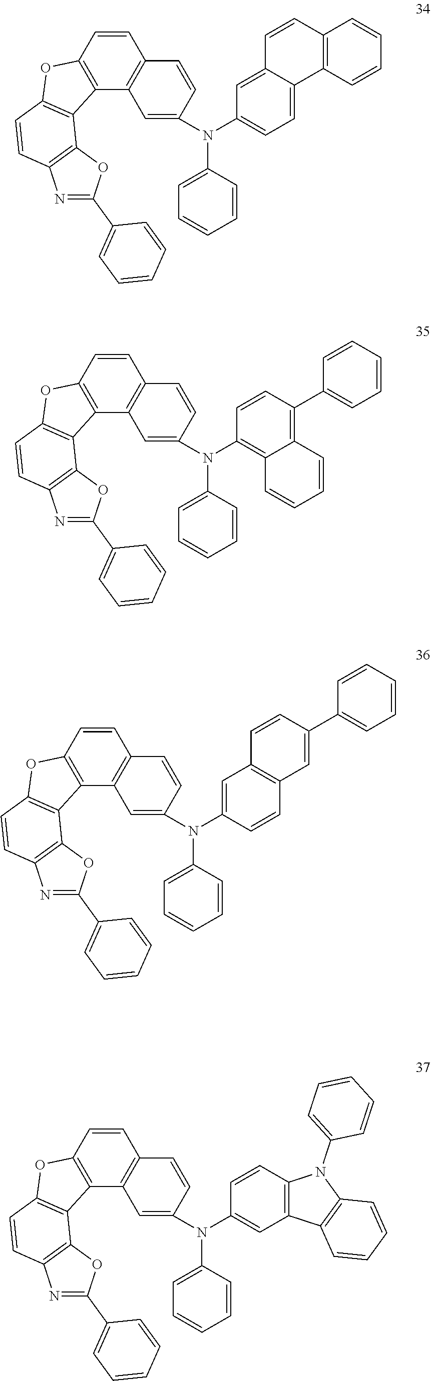 [SMILES: c1ccc(-c2nc3ccc4oc5ccc6ccc(N(c7ccccc7)c7ccc8c(ccc9ccccc98)c7)cc6c5c4c3o2)cc1 ; c1ccc(-c2nc3ccc4oc5ccc6ccc(N(c7ccccc7)c7ccc(-c8ccccc8)c8ccccc78)cc6c5c4c3o2)cc1 ; c1ccc(-c2ccc3cc(N(c4ccccc4)c4ccc5ccc6oc7ccc8nc(-c9ccccc9)oc8c7c6c5c4)ccc3c2)cc1 ; c1ccc(-c2nc3ccc4oc5ccc6ccc(N(c7ccccc7)c7ccc8c(c7)c7ccccc7n8-c7ccccc7)cc6c5c4c3o2)cc1]

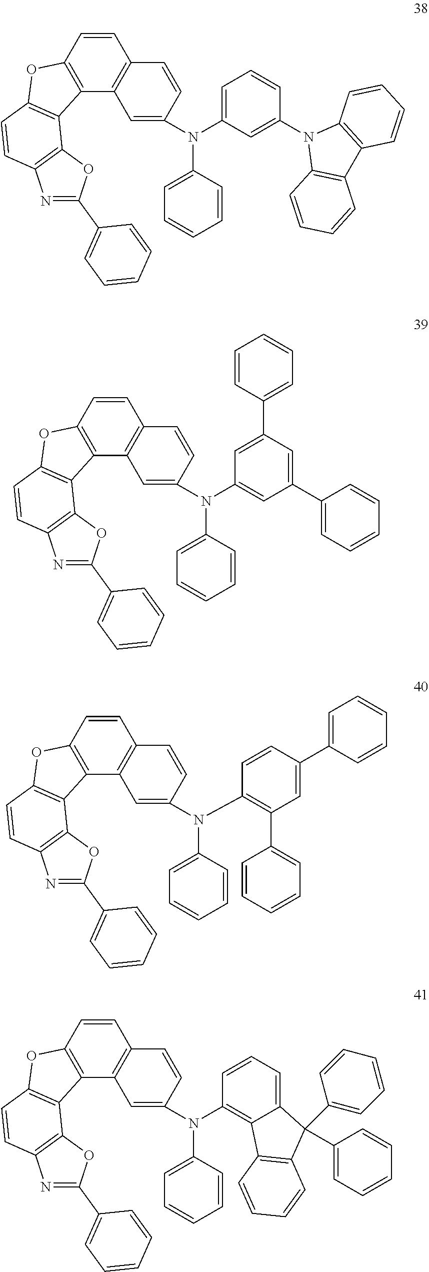 [SMILES: c1ccc(-c2nc3ccc4oc5ccc6ccc(N(c7ccccc7)c7cccc(-n8c9ccccc9c9ccccc98)c7)cc6c5c4c3o2)cc1 ; c1ccc(-c2cc(-c3ccccc3)cc(N(c3ccccc3)c3ccc4ccc5oc6ccc7nc(-c8ccccc8)oc7c6c5c4c3)c2)cc1 ; c1ccc(-c2ccc(N(c3ccccc3)c3ccc4ccc5oc6ccc7nc(-c8ccccc8)oc7c6c5c4c3)c(-c3ccccc3)c2)cc1 ; c1ccc(-c2nc3ccc4oc5ccc6ccc(N(c7ccccc7)c7cccc8c7-c7ccccc7C8(c7ccccc7)c7ccccc7)cc6c5c4c3o2)cc1]

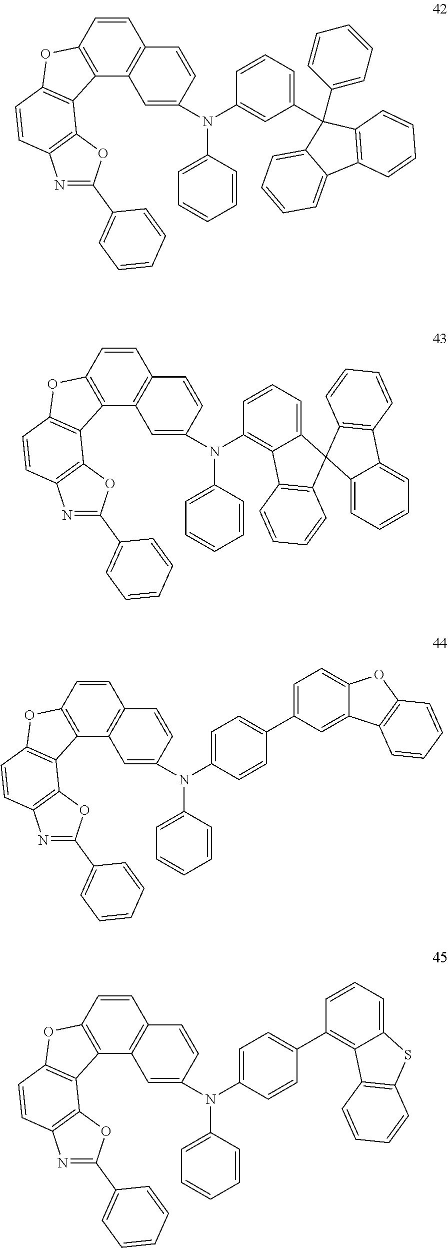 [SMILES: c1ccc(-c2nc3ccc4oc5ccc6ccc(N(c7ccccc7)c7cccc(C8(c9ccccc9)c9ccccc9-c9ccccc98)c7)cc6c5c4c3o2)cc1 ; c1ccc(-c2nc3ccc4oc5ccc6ccc(N(c7ccccc7)c7cccc8c7-c7ccccc7C87c8ccccc8-c8ccccc87)cc6c5c4c3o2)cc1 ; c1ccc(-c2nc3ccc4oc5ccc6ccc(N(c7ccccc7)c7ccc(-c8ccc9oc%10ccccc%10c9c8)cc7)cc6c5c4c3o2)cc1 ; c1ccc(-c2nc3ccc4oc5ccc6ccc(N(c7ccccc7)c7ccc(-c8cccc9sc%10ccccc%10c89)cc7)cc6c5c4c3o2)cc1]

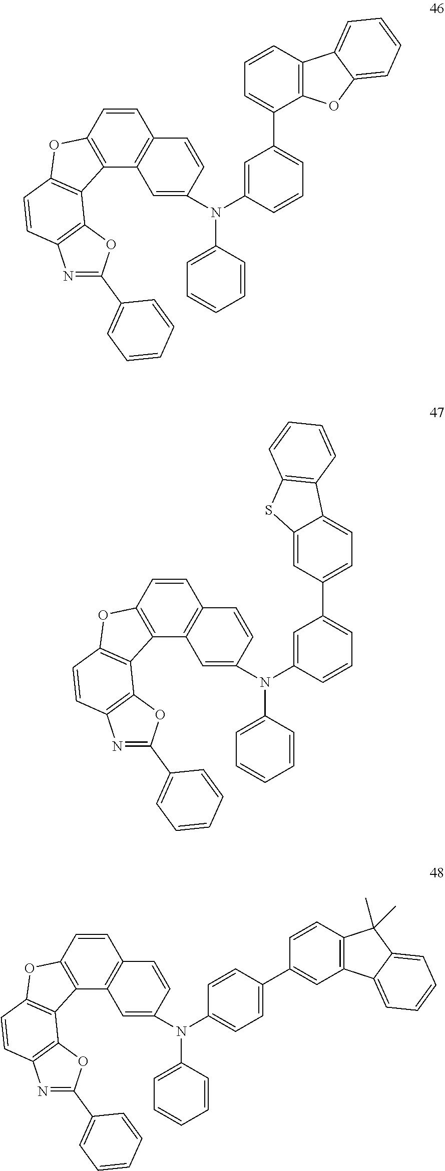 [SMILES: c1ccc(-c2nc3ccc4oc5ccc6ccc(N(c7ccccc7)c7cccc(-c8cccc9c8oc8ccccc89)c7)cc6c5c4c3o2)cc1 ; c1ccc(-c2nc3ccc4oc5ccc6ccc(N(c7ccccc7)c7cccc(-c8ccc9c(c8)sc8ccccc89)c7)cc6c5c4c3o2)cc1 ; CC1(C)c2ccccc2-c2cc(-c3ccc(N(c4ccccc4)c4ccc5ccc6oc7ccc8nc(-c9ccccc9)oc8c7c6c5c4)cc3)ccc21]

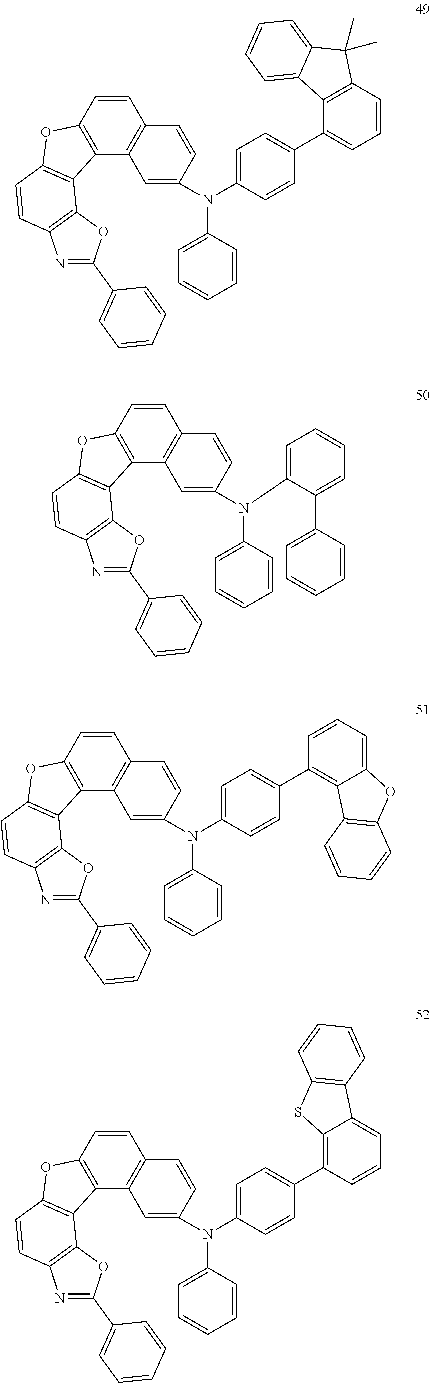 [SMILES: CC1(C)c2ccccc2-c2c(-c3ccc(N(c4ccccc4)c4ccc5ccc6oc7ccc8nc(-c9ccccc9)oc8c7c6c5c4)cc3)cccc21 ; c1ccc(-c2nc3ccc4oc5ccc6ccc(N(c7ccccc7)c7ccccc7-c7ccccc7)cc6c5c4c3o2)cc1 ; c1ccc(-c2nc3ccc4oc5ccc6ccc(N(c7ccccc7)c7ccc(-c8cccc9oc%10ccccc%10c89)cc7)cc6c5c4c3o2)cc1 ; c1ccc(-c2nc3ccc4oc5ccc6ccc(N(c7ccccc7)c7ccc(-c8cccc9c8sc8ccccc89)cc7)cc6c5c4c3o2)cc1]

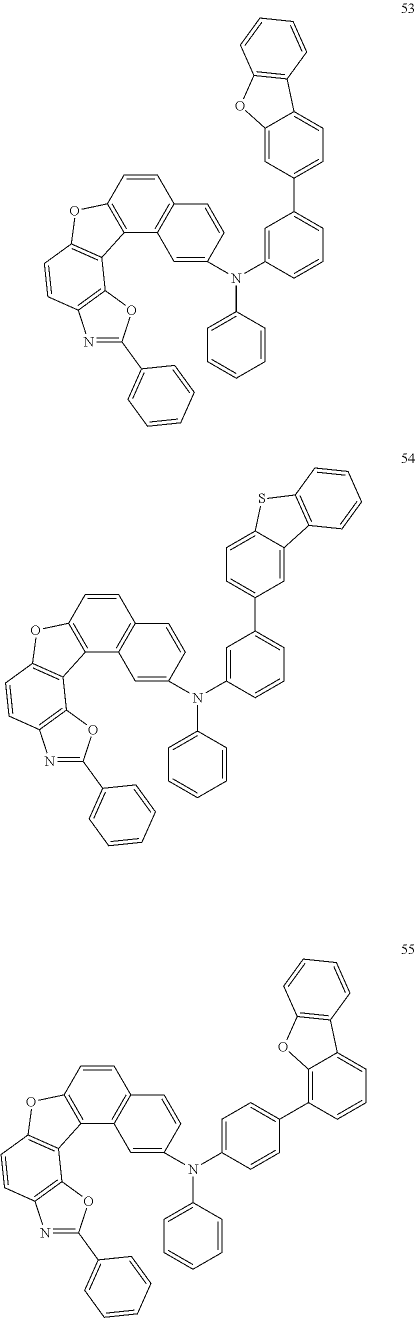 [SMILES: c1ccc(-c2nc3ccc4oc5ccc6ccc(N(c7ccccc7)c7cccc(-c8ccc9c(c8)oc8ccccc89)c7)cc6c5c4c3o2)cc1 ; c1ccc(-c2nc3ccc4oc5ccc6ccc(N(c7ccccc7)c7cccc(-c8ccc9sc%10ccccc%10c9c8)c7)cc6c5c4c3o2)cc1 ; c1ccc(-c2nc3ccc4oc5ccc6ccc(N(c7ccccc7)c7ccc(-c8cccc9c8oc8ccccc89)cc7)cc6c5c4c3o2)cc1]

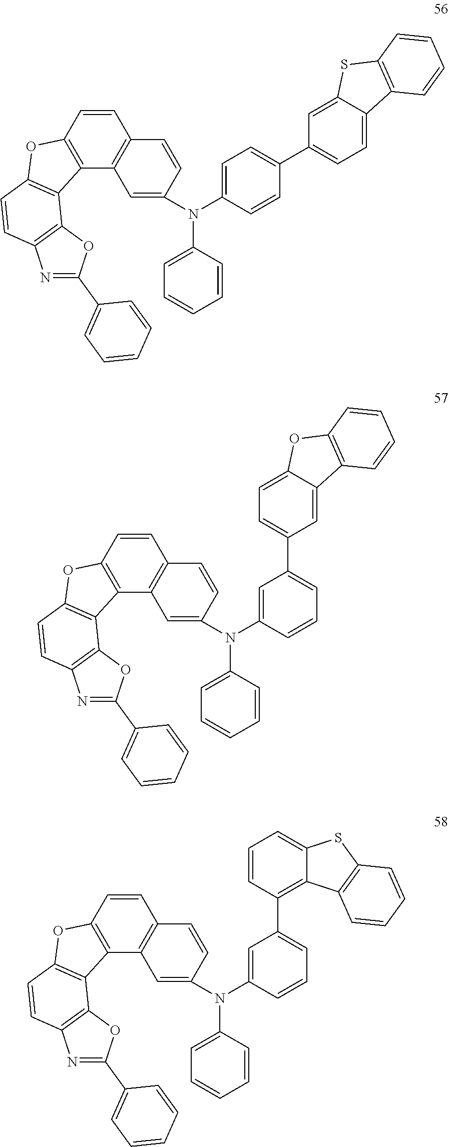 [SMILES: c1ccc(-c2nc3ccc4oc5ccc6ccc(N(c7ccccc7)c7ccc(-c8ccc9c(c8)sc8ccccc89)cc7)cc6c5c4c3o2)cc1 ; c1ccc(-c2nc3ccc4oc5ccc6ccc(N(c7ccccc7)c7cccc(-c8ccc9oc%10ccccc%10c9c8)c7)cc6c5c4c3o2)cc1 ; c1ccc(-c2nc3ccc4oc5ccc6ccc(N(c7ccccc7)c7cccc(-c8cccc9sc%10ccccc%10c89)c7)cc6c5c4c3o2)cc1]

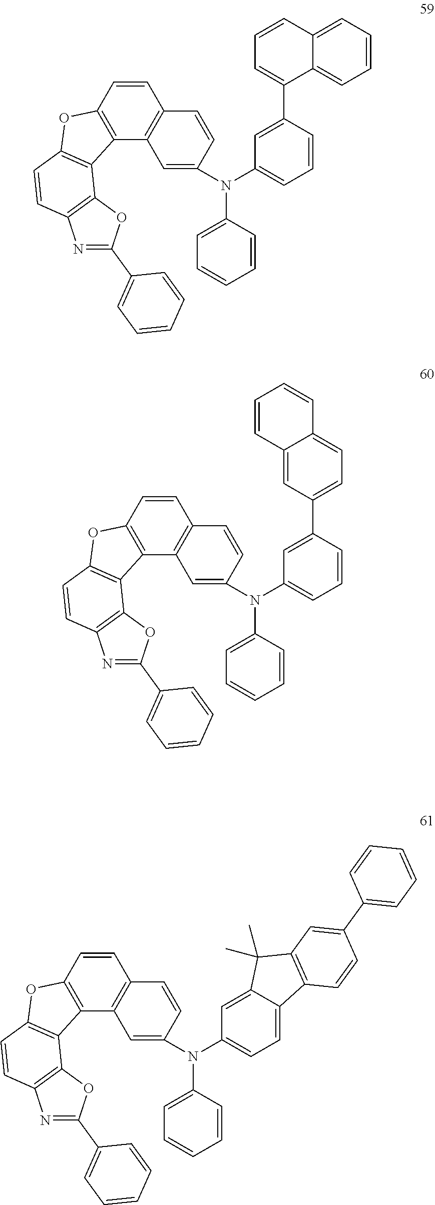 [SMILES: c1ccc(-c2nc3ccc4oc5ccc6ccc(N(c7ccccc7)c7cccc(-c8cccc9ccccc89)c7)cc6c5c4c3o2)cc1 ; c1ccc(-c2nc3ccc4oc5ccc6ccc(N(c7ccccc7)c7cccc(-c8ccc9ccccc9c8)c7)cc6c5c4c3o2)cc1 ; CC1(C)c2cc(-c3ccccc3)ccc2-c2ccc(N(c3ccccc3)c3ccc4ccc5oc6ccc7nc(-c8ccccc8)oc7c6c5c4c3)cc21]

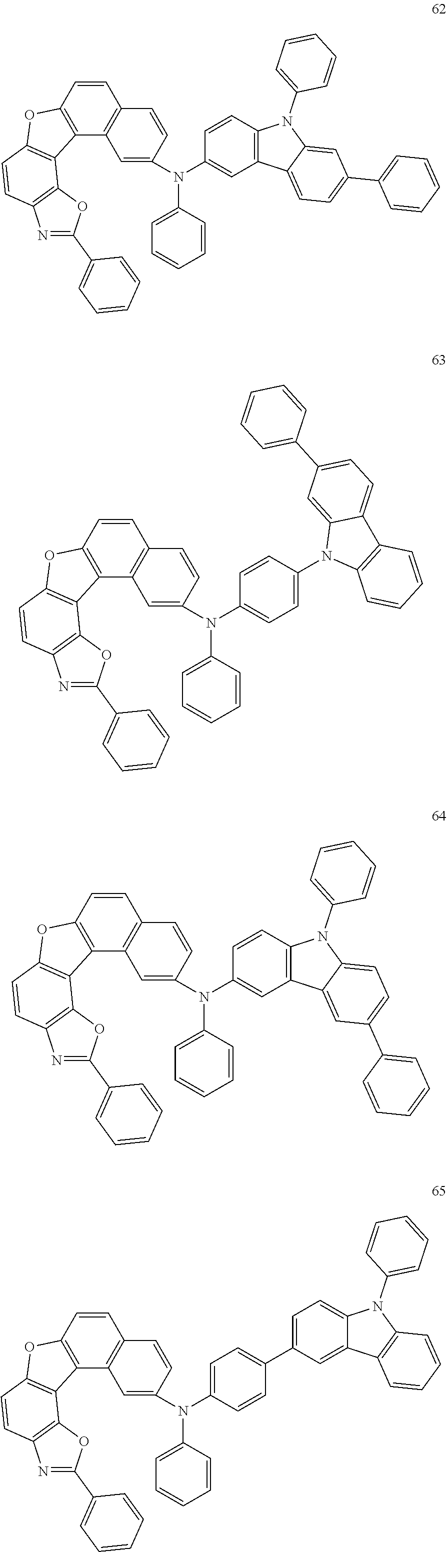 [SMILES: c1ccc(-c2ccc3c4cc(N(c5ccccc5)c5ccc6ccc7oc8ccc9nc(-c%10ccccc%10)oc9c8c7c6c5)ccc4n(-c4ccccc4)c3c2)cc1 ; c1ccc(-c2ccc3c4ccccc4n(-c4ccc(N(c5ccccc5)c5ccc6ccc7oc8ccc9nc(-c%10ccccc%10)oc9c8c7c6c5)cc4)c3c2)cc1 ; c1ccc(-c2ccc3c(c2)c2cc(N(c4ccccc4)c4ccc5ccc6oc7ccc8nc(-c9ccccc9)oc8c7c6c5c4)ccc2n3-c2ccccc2)cc1 ; c1ccc(-c2nc3ccc4oc5ccc6ccc(N(c7ccccc7)c7ccc(-c8ccc9c(c8)c8ccccc8n9-c8ccccc8)cc7)cc6c5c4c3o2)cc1]

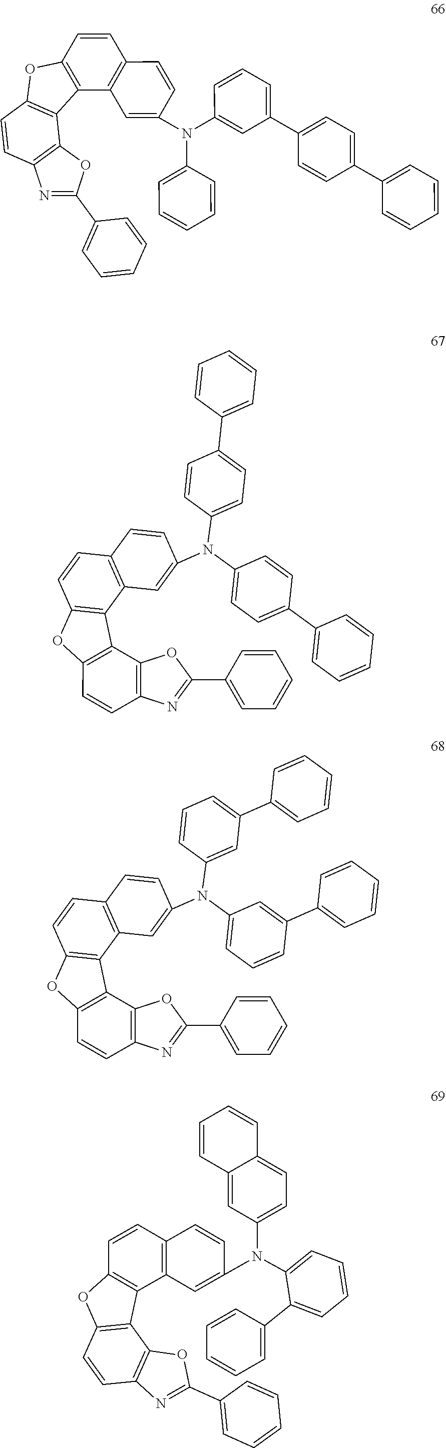 [SMILES: c1ccc(-c2ccc(-c3cccc(N(c4ccccc4)c4ccc5ccc6oc7ccc8nc(-c9ccccc9)oc8c7c6c5c4)c3)cc2)cc1 ; c1ccc(-c2ccc(N(c3ccc(-c4ccccc4)cc3)c3ccc4ccc5oc6ccc7nc(-c8ccccc8)oc7c6c5c4c3)cc2)cc1 ; c1ccc(-c2cccc(N(c3cccc(-c4ccccc4)c3)c3ccc4ccc5oc6ccc7nc(-c8ccccc8)oc7c6c5c4c3)c2)cc1 ; c1ccc(-c2nc3ccc4oc5ccc6ccc(N(c7ccc8ccccc8c7)c7ccccc7-c7ccccc7)cc6c5c4c3o2)cc1]

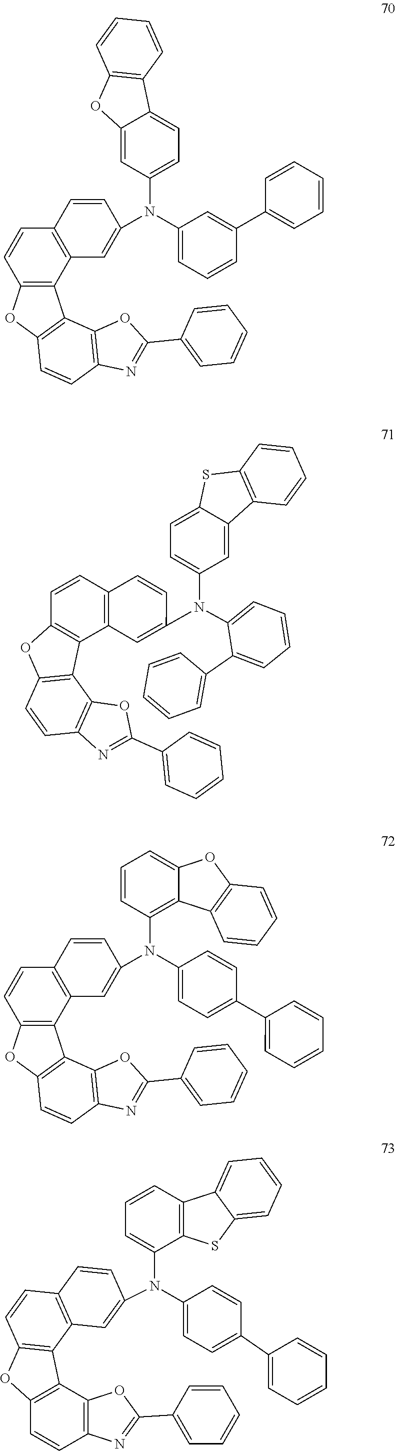 [SMILES: c1ccc(-c2cccc(N(c3ccc4c(c3)oc3ccccc34)c3ccc4ccc5oc6ccc7nc(-c8ccccc8)oc7c6c5c4c3)c2)cc1 ; c1ccc(-c2nc3ccc4oc5ccc6ccc(N(c7ccc8sc9ccccc9c8c7)c7ccccc7-c7ccccc7)cc6c5c4c3o2)cc1 ; c1ccc(-c2ccc(N(c3ccc4ccc5oc6ccc7nc(-c8ccccc8)oc7c6c5c4c3)c3cccc4oc5ccccc5c34)cc2)cc1 ; c1ccc(-c2ccc(N(c3ccc4ccc5oc6ccc7nc(-c8ccccc8)oc7c6c5c4c3)c3cccc4c3sc3ccccc34)cc2)cc1]

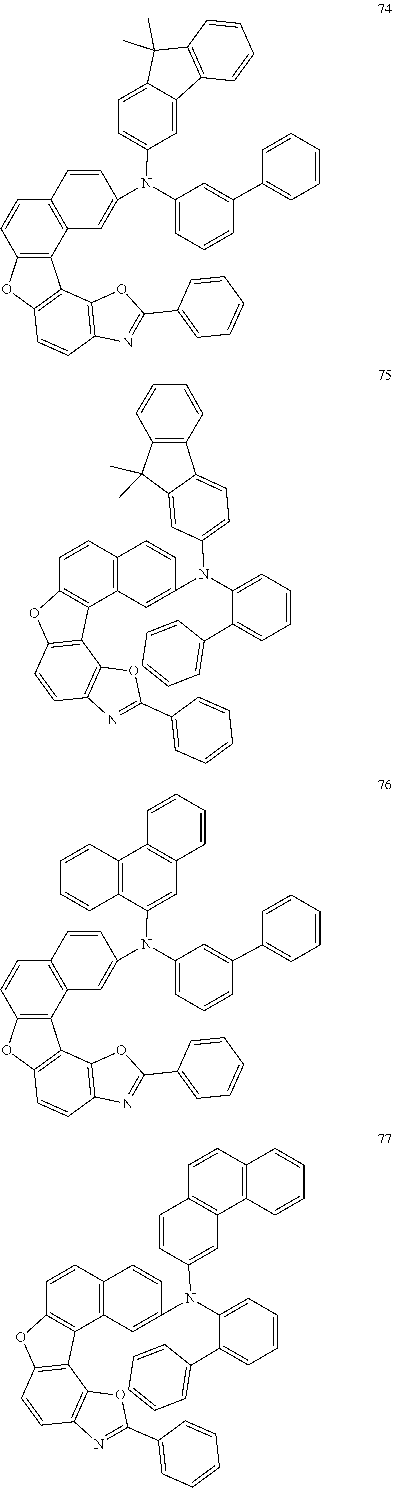

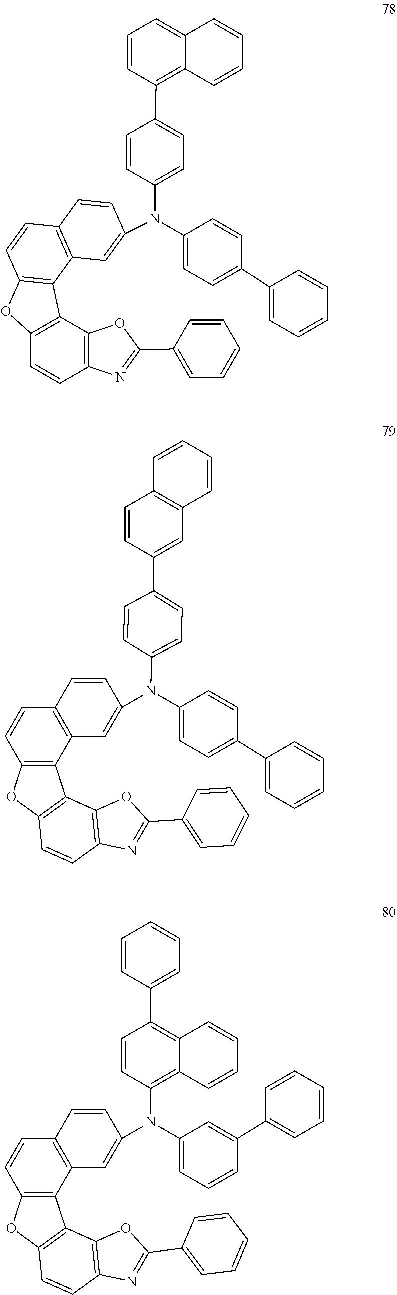 [SMILES: c1ccc(-c2ccc(N(c3ccc(-c4cccc5ccccc45)cc3)c3ccc4ccc5oc6ccc7nc(-c8ccccc8)oc7c6c5c4c3)cc2)cc1 ; c1ccc(-c2ccc(N(c3ccc(-c4ccc5ccccc5c4)cc3)c3ccc4ccc5oc6ccc7nc(-c8ccccc8)oc7c6c5c4c3)cc2)cc1 ; c1ccc(-c2cccc(N(c3ccc4ccc5oc6ccc7nc(-c8ccccc8)oc7c6c5c4c3)c3ccc(-c4ccccc4)c4ccccc34)c2)cc1]

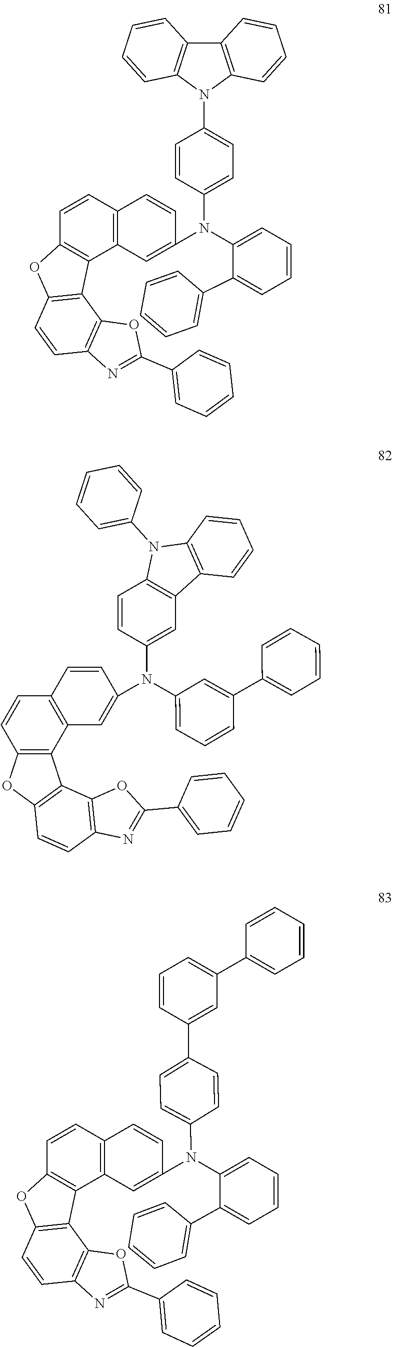 [SMILES: c1ccc(-c2nc3ccc4oc5ccc6ccc(N(c7ccc(-n8c9ccccc9c9ccccc98)cc7)c7ccccc7-c7ccccc7)cc6c5c4c3o2)cc1 ; c1ccc(-c2cccc(N(c3ccc4ccc5oc6ccc7nc(-c8ccccc8)oc7c6c5c4c3)c3ccc4c(c3)c3ccccc3n4-c3ccccc3)c2)cc1 ; c1ccc(-c2cccc(-c3ccc(N(c4ccc5ccc6oc7ccc8nc(-c9ccccc9)oc8c7c6c5c4)c4ccccc4-c4ccccc4)cc3)c2)cc1]

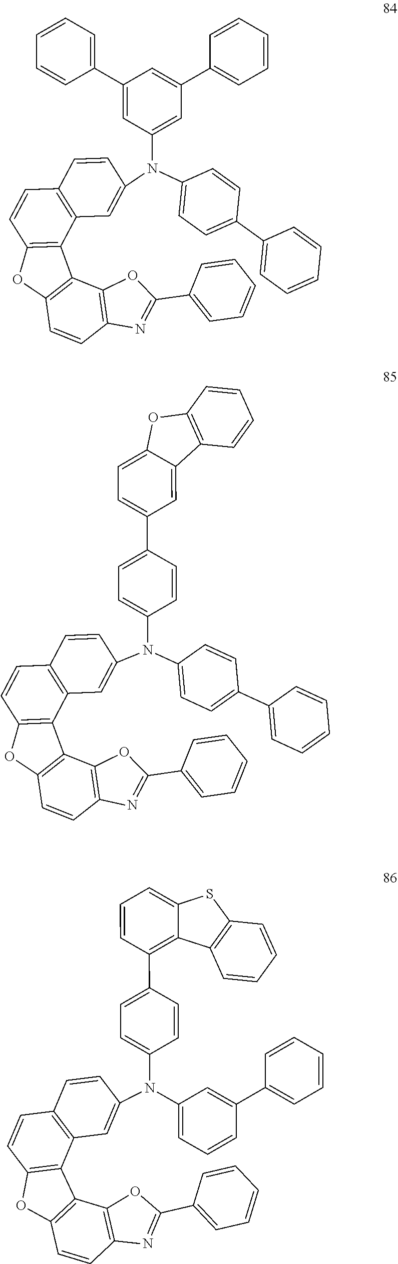 [SMILES: c1ccc(-c2ccc(N(c3cc(-c4ccccc4)cc(-c4ccccc4)c3)c3ccc4ccc5oc6ccc7nc(-c8ccccc8)oc7c6c5c4c3)cc2)cc1 ; c1ccc(-c2ccc(N(c3ccc(-c4ccc5oc6ccccc6c5c4)cc3)c3ccc4ccc5oc6ccc7nc(-c8ccccc8)oc7c6c5c4c3)cc2)cc1 ; c1ccc(-c2cccc(N(c3ccc(-c4cccc5sc6ccccc6c45)cc3)c3ccc4ccc5oc6ccc7nc(-c8ccccc8)oc7c6c5c4c3)c2)cc1]

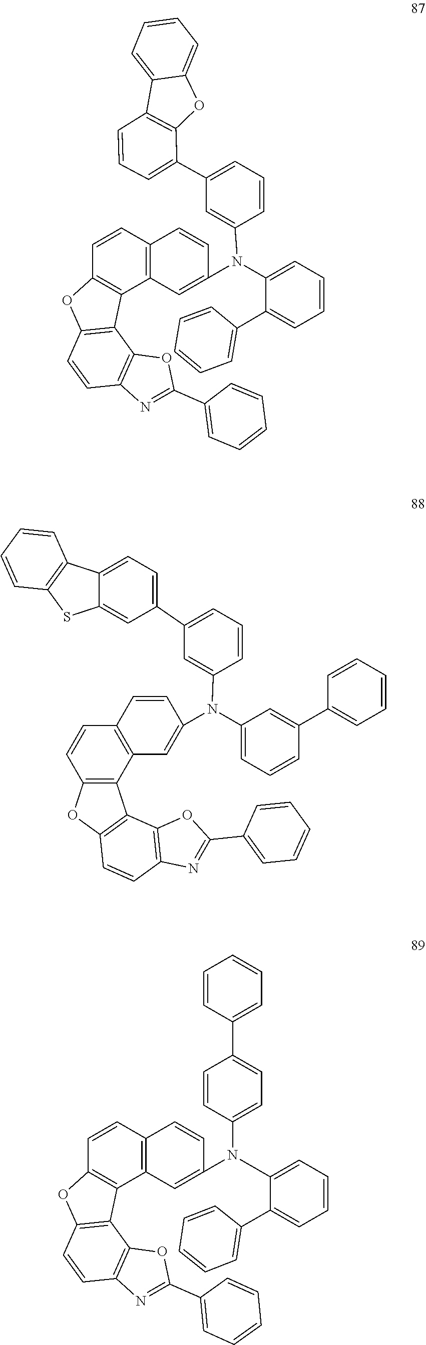 [SMILES: c1ccc(-c2nc3ccc4oc5ccc6ccc(N(c7cccc(-c8cccc9c8oc8ccccc89)c7)c7ccccc7-c7ccccc7)cc6c5c4c3o2)cc1 ; c1ccc(-c2cccc(N(c3cccc(-c4ccc5c(c4)sc4ccccc45)c3)c3ccc4ccc5oc6ccc7nc(-c8ccccc8)oc7c6c5c4c3)c2)cc1 ; c1ccc(-c2ccc(N(c3ccc4ccc5oc6ccc7nc(-c8ccccc8)oc7c6c5c4c3)c3ccccc3-c3ccccc3)cc2)cc1]

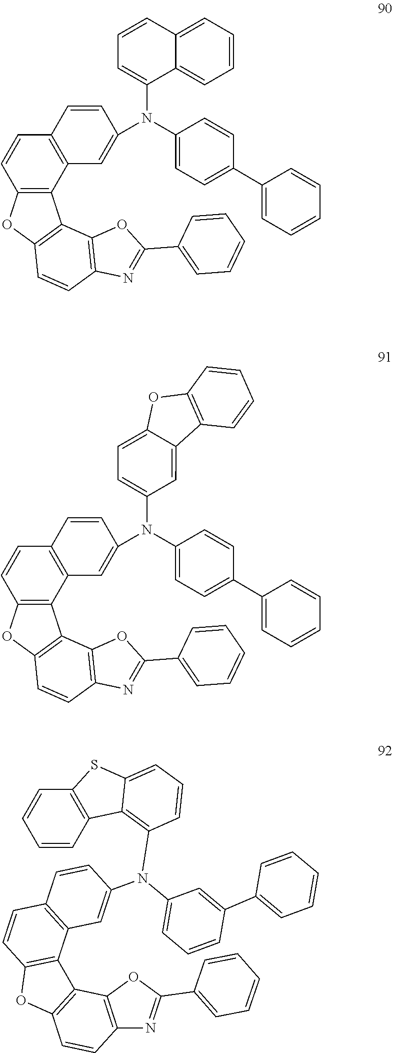 [SMILES: c1ccc(-c2ccc(N(c3ccc4ccc5oc6ccc7nc(-c8ccccc8)oc7c6c5c4c3)c3cccc4ccccc34)cc2)cc1 ; c1ccc(-c2ccc(N(c3ccc4oc5ccccc5c4c3)c3ccc4ccc5oc6ccc7nc(-c8ccccc8)oc7c6c5c4c3)cc2)cc1 ; c1ccc(-c2cccc(N(c3ccc4ccc5oc6ccc7nc(-c8ccccc8)oc7c6c5c4c3)c3cccc4sc5ccccc5c34)c2)cc1]

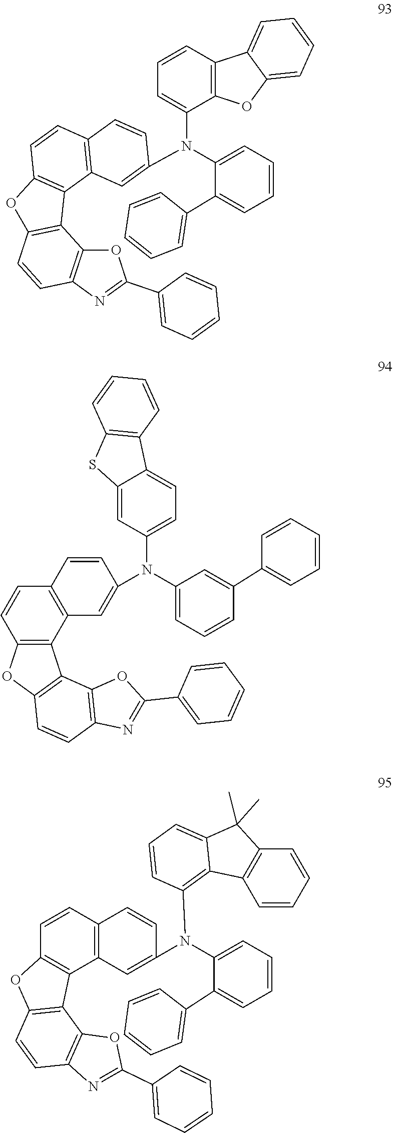 [SMILES: c1ccc(-c2nc3ccc4oc5ccc6ccc(N(c7ccccc7-c7ccccc7)c7cccc8c7oc7ccccc78)cc6c5c4c3o2)cc1 ; c1ccc(-c2cccc(N(c3ccc4c(c3)sc3ccccc34)c3ccc4ccc5oc6ccc7nc(-c8ccccc8)oc7c6c5c4c3)c2)cc1 ; CC1(C)c2ccccc2-c2c(N(c3ccc4ccc5oc6ccc7nc(-c8ccccc8)oc7c6c5c4c3)c3ccccc3-c3ccccc3)cccc21]

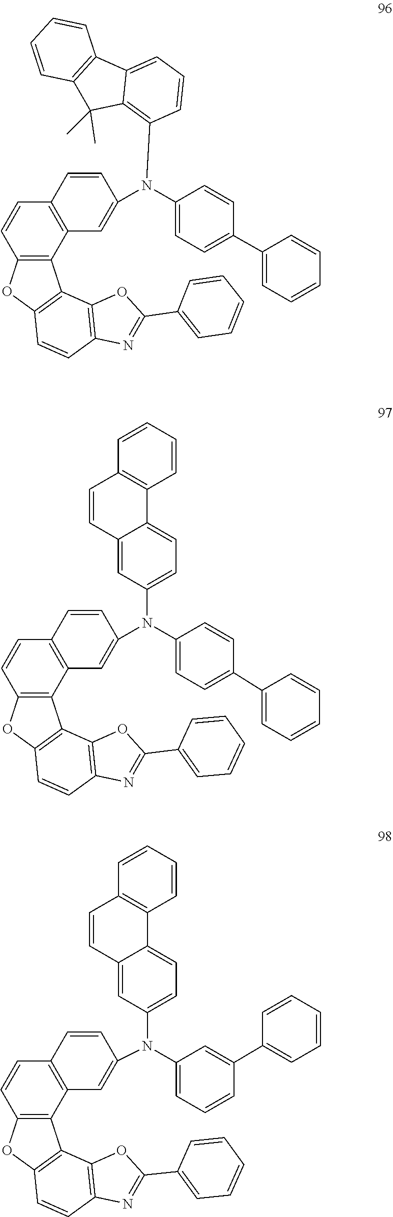 [SMILES: CC1(C)c2ccccc2-c2cccc(N(c3ccc(-c4ccccc4)cc3)c3ccc4ccc5oc6ccc7nc(-c8ccccc8)oc7c6c5c4c3)c21 ; c1ccc(-c2ccc(N(c3ccc4c(ccc5ccccc54)c3)c3ccc4ccc5oc6ccc7nc(-c8ccccc8)oc7c6c5c4c3)cc2)cc1 ; c1ccc(-c2cccc(N(c3ccc4c(ccc5ccccc54)c3)c3ccc4ccc5oc6ccc7nc(-c8ccccc8)oc7c6c5c4c3)c2)cc1]

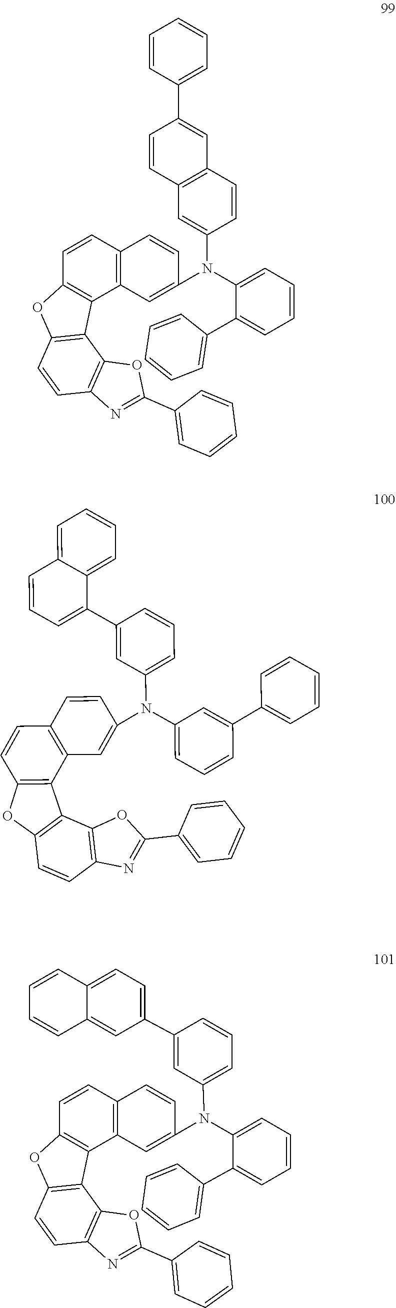 [SMILES: c1ccc(-c2ccc3cc(N(c4ccc5ccc6oc7ccc8nc(-c9ccccc9)oc8c7c6c5c4)c4ccccc4-c4ccccc4)ccc3c2)cc1 ; c1ccc(-c2cccc(N(c3cccc(-c4cccc5ccccc45)c3)c3ccc4ccc5oc6ccc7nc(-c8ccccc8)oc7c6c5c4c3)c2)cc1 ; c1ccc(-c2nc3ccc4oc5ccc6ccc(N(c7cccc(-c8ccc9ccccc9c8)c7)c7ccccc7-c7ccccc7)cc6c5c4c3o2)cc1]

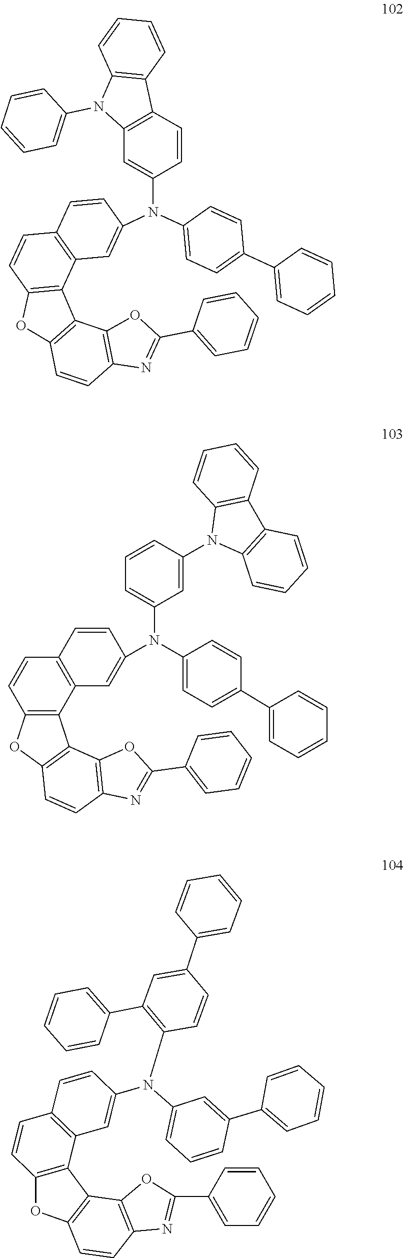 [SMILES: c1ccc(-c2ccc(N(c3ccc4ccc5oc6ccc7nc(-c8ccccc8)oc7c6c5c4c3)c3ccc4c5ccccc5n(-c5ccccc5)c4c3)cc2)cc1 ; c1ccc(-c2ccc(N(c3cccc(-n4c5ccccc5c5ccccc54)c3)c3ccc4ccc5oc6ccc7nc(-c8ccccc8)oc7c6c5c4c3)cc2)cc1 ; c1ccc(-c2cccc(N(c3ccc4ccc5oc6ccc7nc(-c8ccccc8)oc7c6c5c4c3)c3ccc(-c4ccccc4)cc3-c3ccccc3)c2)cc1]

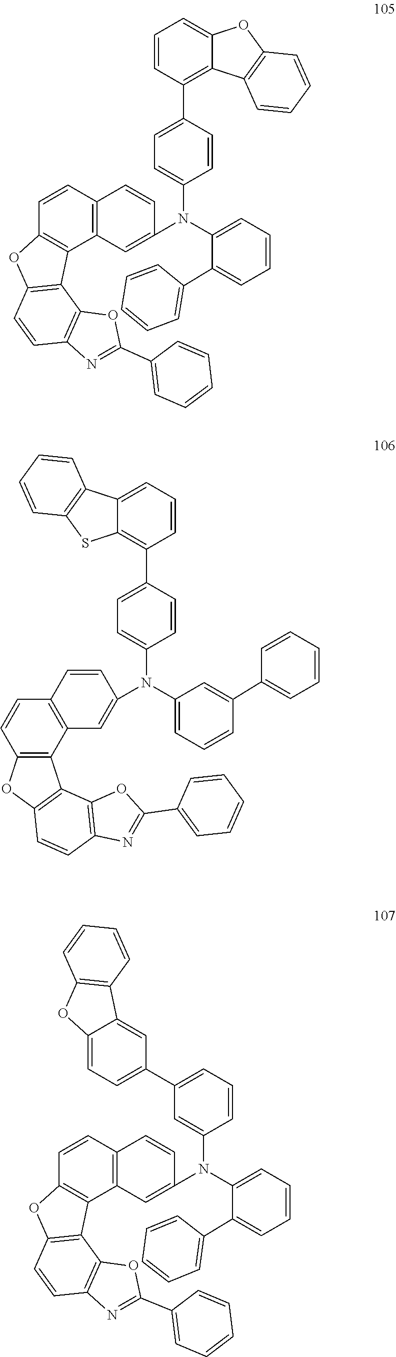 [SMILES: c1ccc(-c2nc3ccc4oc5ccc6ccc(N(c7ccc(-c8cccc9oc%10ccccc%10c89)cc7)c7ccccc7-c7ccccc7)cc6c5c4c3o2)cc1 ; c1ccc(-c2cccc(N(c3ccc(-c4cccc5c4sc4ccccc45)cc3)c3ccc4ccc5oc6ccc7nc(-c8ccccc8)oc7c6c5c4c3)c2)cc1 ; c1ccc(-c2nc3ccc4oc5ccc6ccc(N(c7cccc(-c8ccc9oc%10ccccc%10c9c8)c7)c7ccccc7-c7ccccc7)cc6c5c4c3o2)cc1]

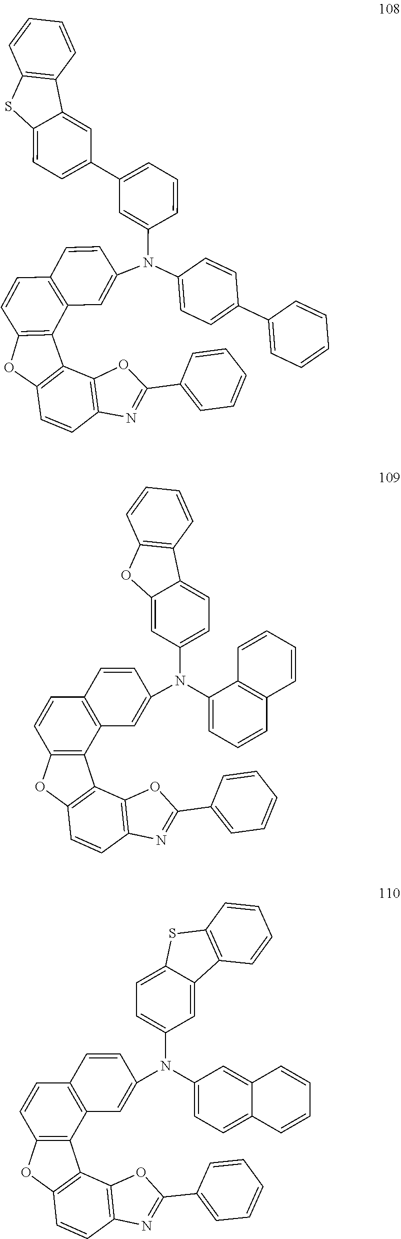 [SMILES: c1ccc(-c2ccc(N(c3cccc(-c4ccc5sc6ccccc6c5c4)c3)c3ccc4ccc5oc6ccc7nc(-c8ccccc8)oc7c6c5c4c3)cc2)cc1 ; c1ccc(-c2nc3ccc4oc5ccc6ccc(N(c7ccc8c(c7)oc7ccccc78)c7cccc8ccccc78)cc6c5c4c3o2)cc1 ; c1ccc(-c2nc3ccc4oc5ccc6ccc(N(c7ccc8ccccc8c7)c7ccc8sc9ccccc9c8c7)cc6c5c4c3o2)cc1]

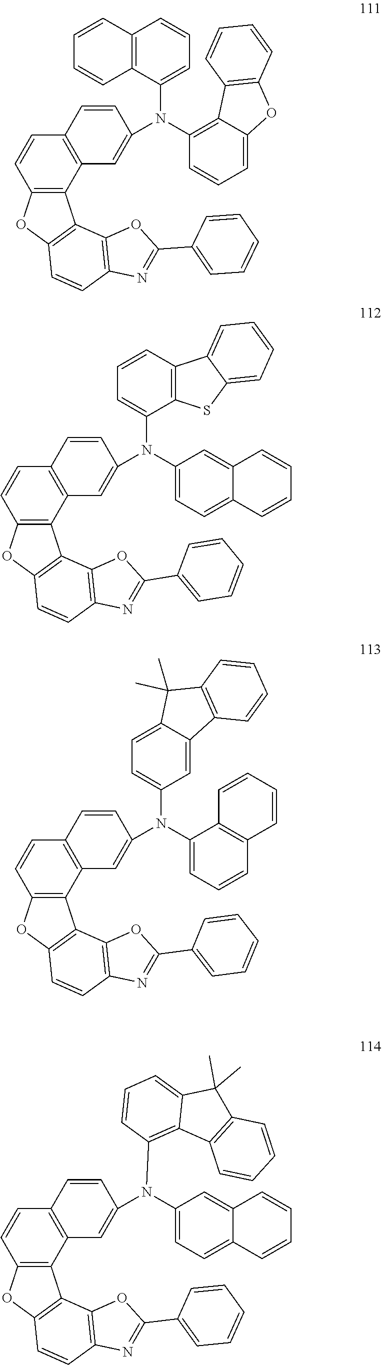 [SMILES: c1ccc(-c2nc3ccc4oc5ccc6ccc(N(c7cccc8ccccc78)c7cccc8oc9ccccc9c78)cc6c5c4c3o2)cc1 ; c1ccc(-c2nc3ccc4oc5ccc6ccc(N(c7ccc8ccccc8c7)c7cccc8c7sc7ccccc78)cc6c5c4c3o2)cc1 ; CC1(C)c2ccccc2-c2cc(N(c3ccc4ccc5oc6ccc7nc(-c8ccccc8)oc7c6c5c4c3)c3cccc4ccccc34)ccc21 ; CC1(C)c2ccccc2-c2c(N(c3ccc4ccccc4c3)c3ccc4ccc5oc6ccc7nc(-c8ccccc8)oc7c6c5c4c3)cccc21]

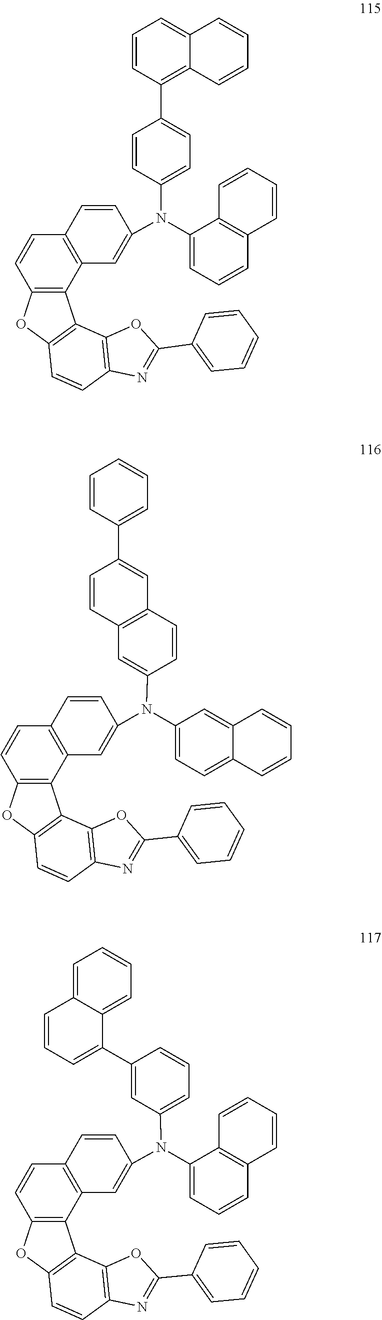 [SMILES: c1ccc(-c2nc3ccc4oc5ccc6ccc(N(c7ccc(-c8cccc9ccccc89)cc7)c7cccc8ccccc78)cc6c5c4c3o2)cc1 ; c1ccc(-c2ccc3cc(N(c4ccc5ccccc5c4)c4ccc5ccc6oc7ccc8nc(-c9ccccc9)oc8c7c6c5c4)ccc3c2)cc1 ; c1ccc(-c2nc3ccc4oc5ccc6ccc(N(c7cccc(-c8cccc9ccccc89)c7)c7cccc8ccccc78)cc6c5c4c3o2)cc1]

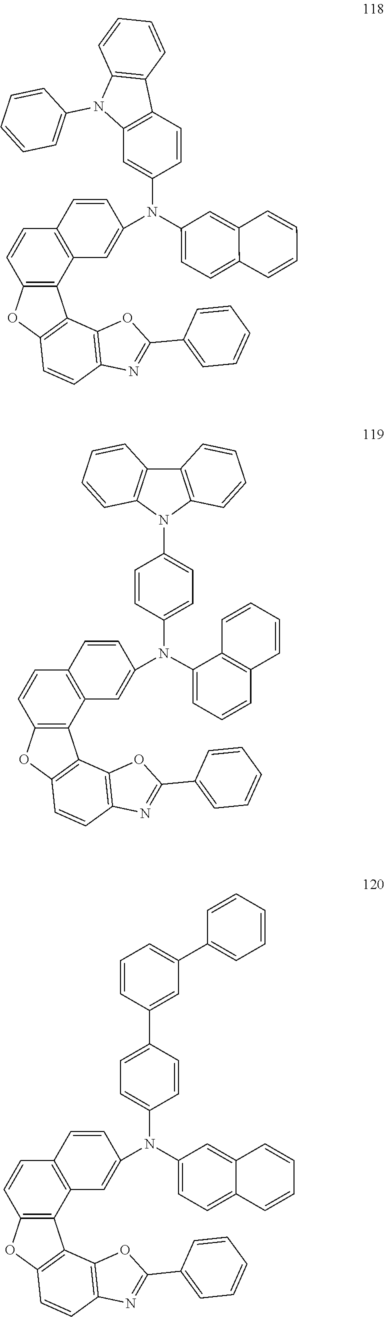 [SMILES: c1ccc(-c2nc3ccc4oc5ccc6ccc(N(c7ccc8ccccc8c7)c7ccc8c9ccccc9n(-c9ccccc9)c8c7)cc6c5c4c3o2)cc1 ; c1ccc(-c2nc3ccc4oc5ccc6ccc(N(c7ccc(-n8c9ccccc9c9ccccc98)cc7)c7cccc8ccccc78)cc6c5c4c3o2)cc1 ; c1ccc(-c2cccc(-c3ccc(N(c4ccc5ccccc5c4)c4ccc5ccc6oc7ccc8nc(-c9ccccc9)oc8c7c6c5c4)cc3)c2)cc1]

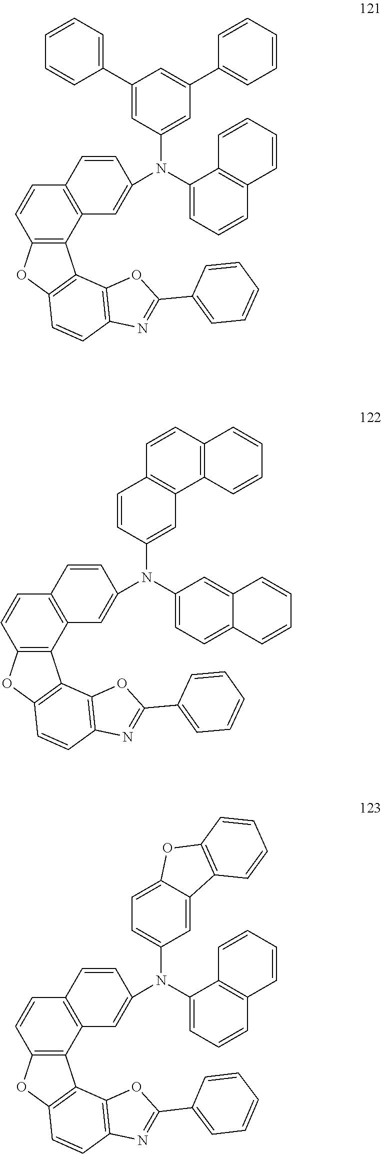 [SMILES: c1ccc(-c2cc(-c3ccccc3)cc(N(c3ccc4ccc5oc6ccc7nc(-c8ccccc8)oc7c6c5c4c3)c3cccc4ccccc34)c2)cc1 ; c1ccc(-c2nc3ccc4oc5ccc6ccc(N(c7ccc8ccccc8c7)c7ccc8ccc9ccccc9c8c7)cc6c5c4c3o2)cc1 ; c1ccc(-c2nc3ccc4oc5ccc6ccc(N(c7ccc8oc9ccccc9c8c7)c7cccc8ccccc78)cc6c5c4c3o2)cc1]

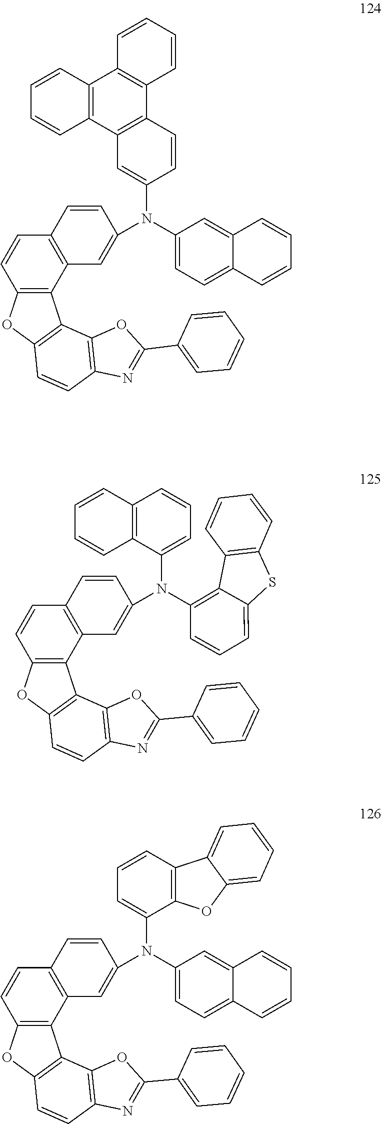 [SMILES: c1ccc(-c2nc3ccc4oc5ccc6ccc(N(c7ccc8ccccc8c7)c7ccc8c9ccccc9c9ccccc9c8c7)cc6c5c4c3o2)cc1 ; c1ccc(-c2nc3ccc4oc5ccc6ccc(N(c7cccc8ccccc78)c7cccc8sc9ccccc9c78)cc6c5c4c3o2)cc1 ; c1ccc(-c2nc3ccc4oc5ccc6ccc(N(c7ccc8ccccc8c7)c7cccc8c7oc7ccccc78)cc6c5c4c3o2)cc1]

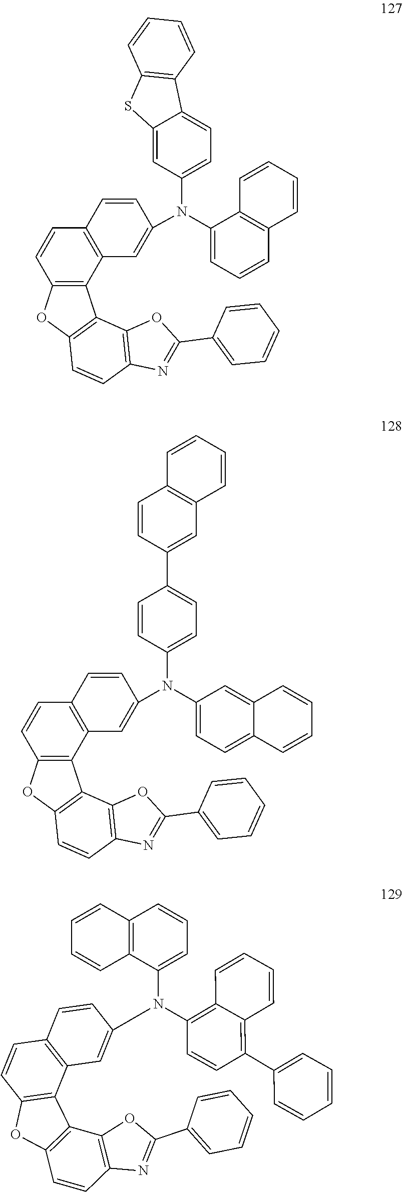 [SMILES: c1ccc(-c2nc3ccc4oc5ccc6ccc(N(c7ccc8c(c7)sc7ccccc78)c7cccc8ccccc78)cc6c5c4c3o2)cc1 ; c1ccc(-c2nc3ccc4oc5ccc6ccc(N(c7ccc(-c8ccc9ccccc9c8)cc7)c7ccc8ccccc8c7)cc6c5c4c3o2)cc1 ; c1ccc(-c2nc3ccc4oc5ccc6ccc(N(c7cccc8ccccc78)c7ccc(-c8ccccc8)c8ccccc78)cc6c5c4c3o2)cc1]

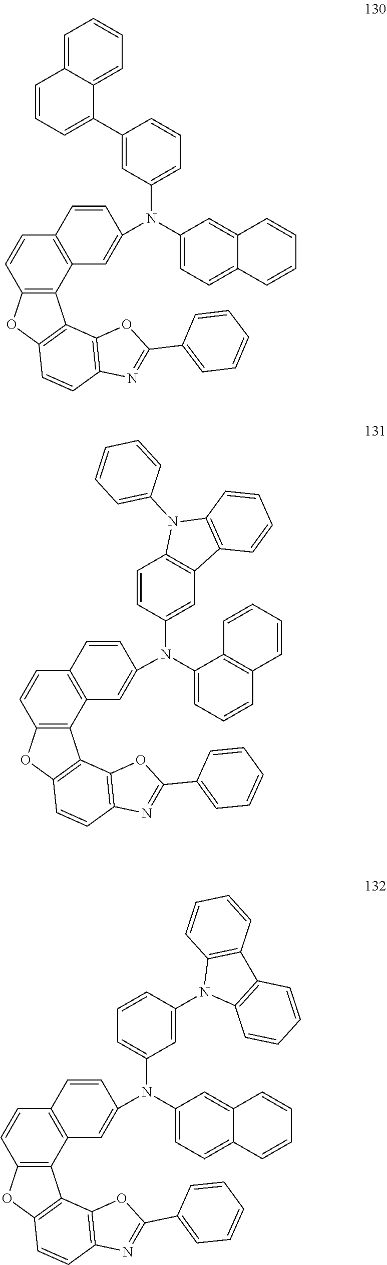 [SMILES: c1ccc(-c2nc3ccc4oc5ccc6ccc(N(c7cccc(-c8cccc9ccccc89)c7)c7ccc8ccccc8c7)cc6c5c4c3o2)cc1 ; c1ccc(-c2nc3ccc4oc5ccc6ccc(N(c7ccc8c(c7)c7ccccc7n8-c7ccccc7)c7cccc8ccccc78)cc6c5c4c3o2)cc1 ; c1ccc(-c2nc3ccc4oc5ccc6ccc(N(c7cccc(-n8c9ccccc9c9ccccc98)c7)c7ccc8ccccc8c7)cc6c5c4c3o2)cc1]

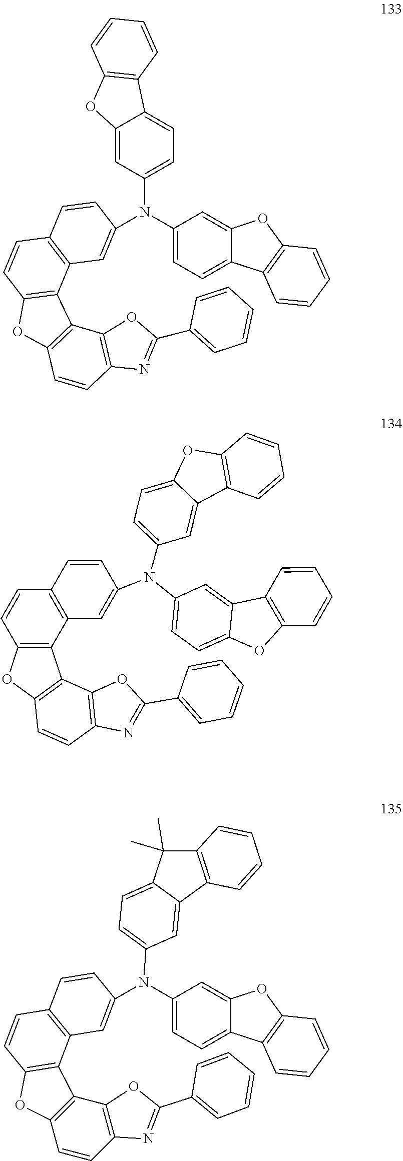 [SMILES: c1ccc(-c2nc3ccc4oc5ccc6ccc(N(c7ccc8c(c7)oc7ccccc78)c7ccc8c(c7)oc7ccccc78)cc6c5c4c3o2)cc1 ; c1ccc(-c2nc3ccc4oc5ccc6ccc(N(c7ccc8oc9ccccc9c8c7)c7ccc8oc9ccccc9c8c7)cc6c5c4c3o2)cc1 ; CC1(C)c2ccccc2-c2cc(N(c3ccc4c(c3)oc3ccccc34)c3ccc4ccc5oc6ccc7nc(-c8ccccc8)oc7c6c5c4c3)ccc21]

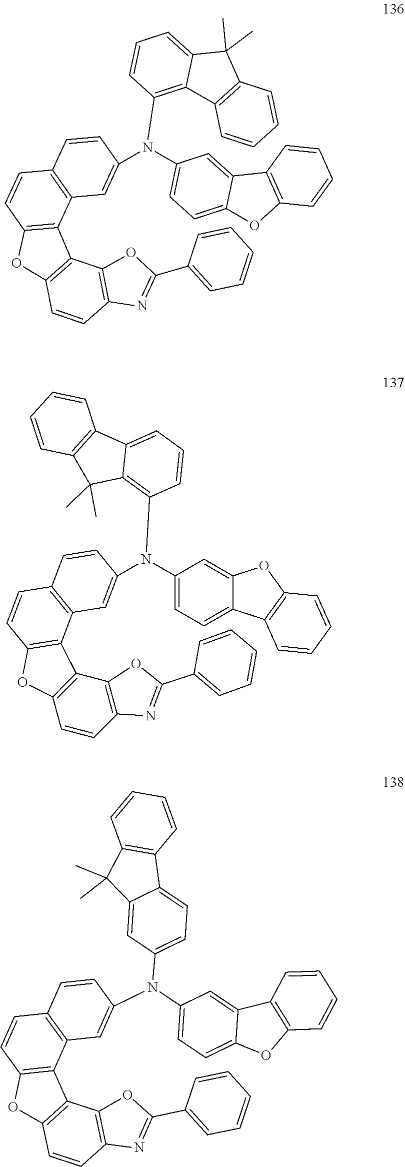 [SMILES: CC1(C)c2ccccc2-c2c(N(c3ccc4oc5ccccc5c4c3)c3ccc4ccc5oc6ccc7nc(-c8ccccc8)oc7c6c5c4c3)cccc21 ; CC1(C)c2ccccc2-c2cccc(N(c3ccc4c(c3)oc3ccccc34)c3ccc4ccc5oc6ccc7nc(-c8ccccc8)oc7c6c5c4c3)c21 ; CC1(C)c2ccccc2-c2ccc(N(c3ccc4oc5ccccc5c4c3)c3ccc4ccc5oc6ccc7nc(-c8ccccc8)oc7c6c5c4c3)cc21]

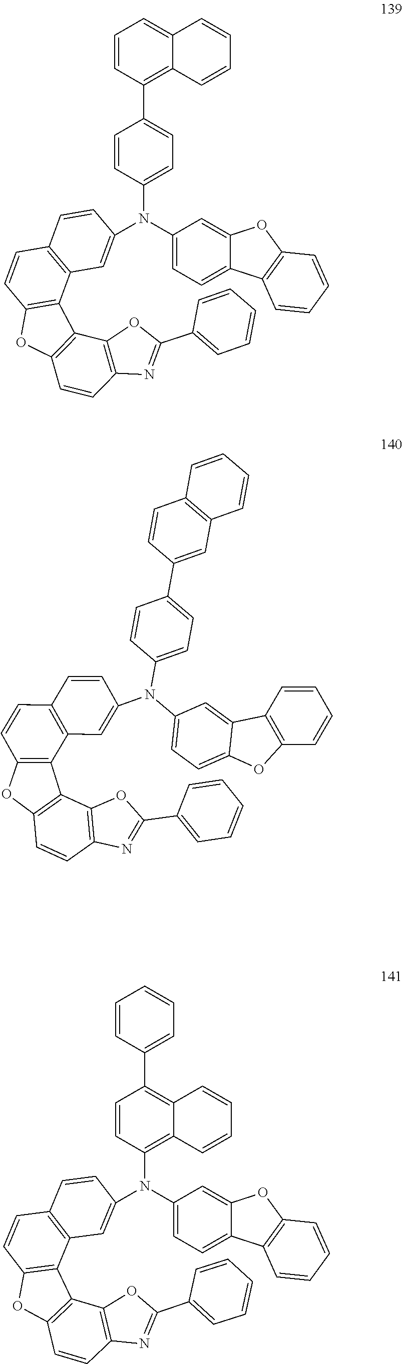 [SMILES: c1ccc(-c2nc3ccc4oc5ccc6ccc(N(c7ccc(-c8cccc9ccccc89)cc7)c7ccc8c(c7)oc7ccccc78)cc6c5c4c3o2)cc1 ; c1ccc(-c2nc3ccc4oc5ccc6ccc(N(c7ccc(-c8ccc9ccccc9c8)cc7)c7ccc8oc9ccccc9c8c7)cc6c5c4c3o2)cc1 ; c1ccc(-c2nc3ccc4oc5ccc6ccc(N(c7ccc8c(c7)oc7ccccc78)c7ccc(-c8ccccc8)c8ccccc78)cc6c5c4c3o2)cc1]

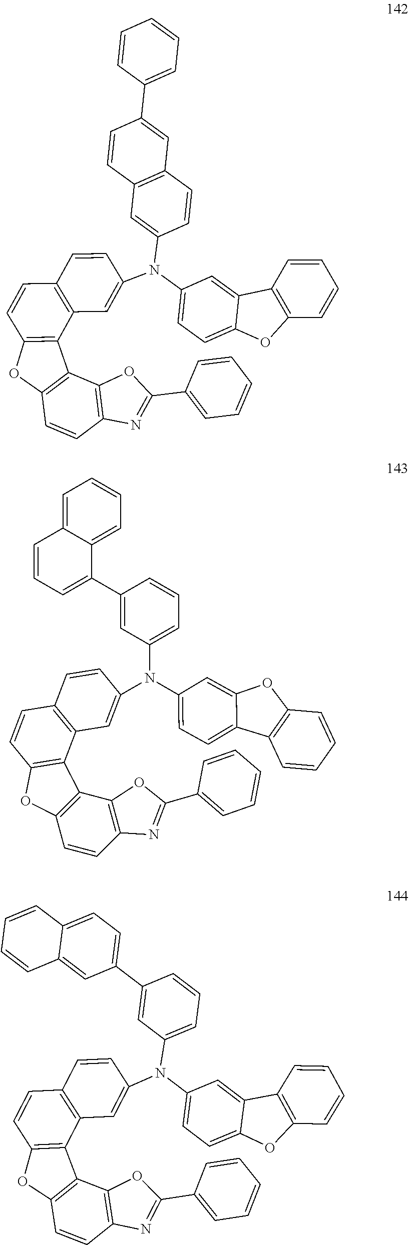

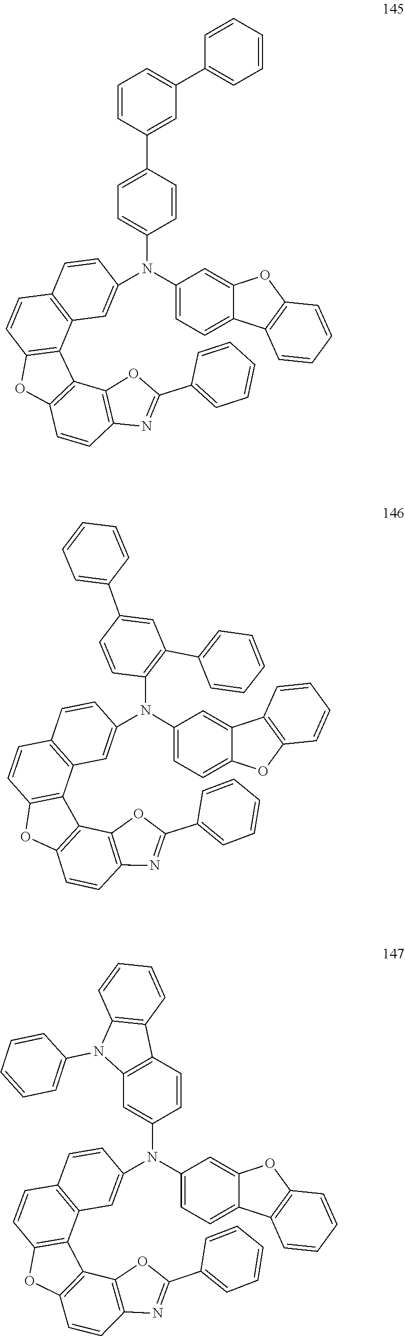 [SMILES: c1ccc(-c2cccc(-c3ccc(N(c4ccc5c(c4)oc4ccccc45)c4ccc5ccc6oc7ccc8nc(-c9ccccc9)oc8c7c6c5c4)cc3)c2)cc1 ; c1ccc(-c2ccc(N(c3ccc4oc5ccccc5c4c3)c3ccc4ccc5oc6ccc7nc(-c8ccccc8)oc7c6c5c4c3)c(-c3ccccc3)c2)cc1 ; c1ccc(-c2nc3ccc4oc5ccc6ccc(N(c7ccc8c(c7)oc7ccccc78)c7ccc8c9ccccc9n(-c9ccccc9)c8c7)cc6c5c4c3o2)cc1]

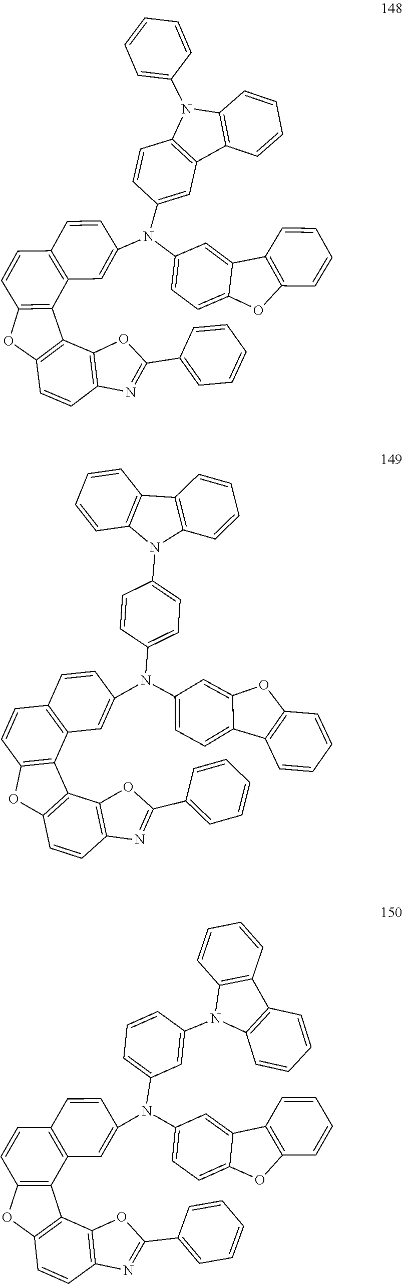 [SMILES: c1ccc(-c2nc3ccc4oc5ccc6ccc(N(c7ccc8oc9ccccc9c8c7)c7ccc8c(c7)c7ccccc7n8-c7ccccc7)cc6c5c4c3o2)cc1 ; c1ccc(-c2nc3ccc4oc5ccc6ccc(N(c7ccc(-n8c9ccccc9c9ccccc98)cc7)c7ccc8c(c7)oc7ccccc78)cc6c5c4c3o2)cc1 ; c1ccc(-c2nc3ccc4oc5ccc6ccc(N(c7cccc(-n8c9ccccc9c9ccccc98)c7)c7ccc8oc9ccccc9c8c7)cc6c5c4c3o2)cc1]

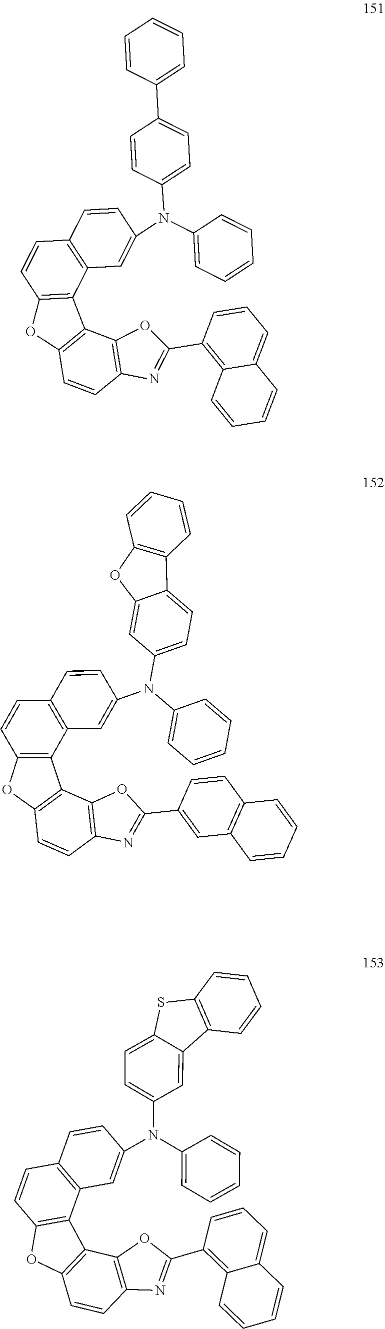 [SMILES: c1ccc(-c2ccc(N(c3ccccc3)c3ccc4ccc5oc6ccc7nc(-c8cccc9ccccc89)oc7c6c5c4c3)cc2)cc1 ; c1ccc(N(c2ccc3c(c2)oc2ccccc23)c2ccc3ccc4oc5ccc6nc(-c7ccc8ccccc8c7)oc6c5c4c3c2)cc1 ; c1ccc(N(c2ccc3sc4ccccc4c3c2)c2ccc3ccc4oc5ccc6nc(-c7cccc8ccccc78)oc6c5c4c3c2)cc1]

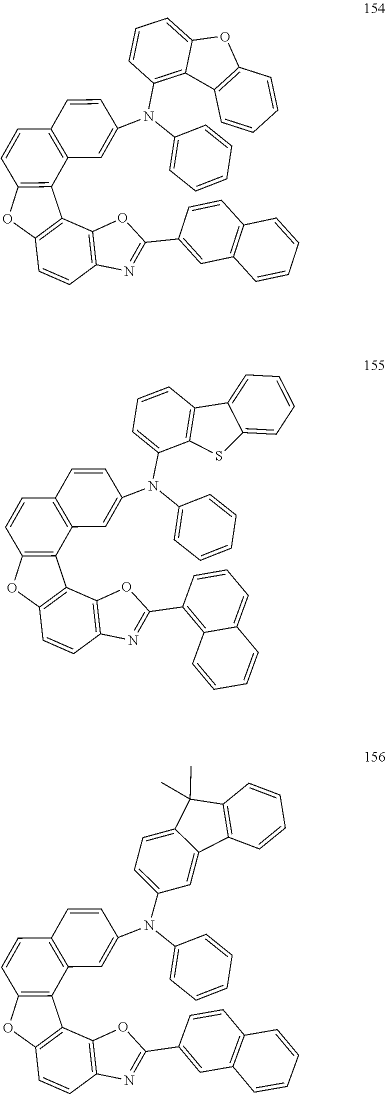 [SMILES: c1ccc(N(c2ccc3ccc4oc5ccc6nc(-c7ccc8ccccc8c7)oc6c5c4c3c2)c2cccc3oc4ccccc4c23)cc1 ; c1ccc(N(c2ccc3ccc4oc5ccc6nc(-c7cccc8ccccc78)oc6c5c4c3c2)c2cccc3c2sc2ccccc23)cc1 ; CC1(C)c2ccccc2-c2cc(N(c3ccccc3)c3ccc4ccc5oc6ccc7nc(-c8ccc9ccccc9c8)oc7c6c5c4c3)ccc21]

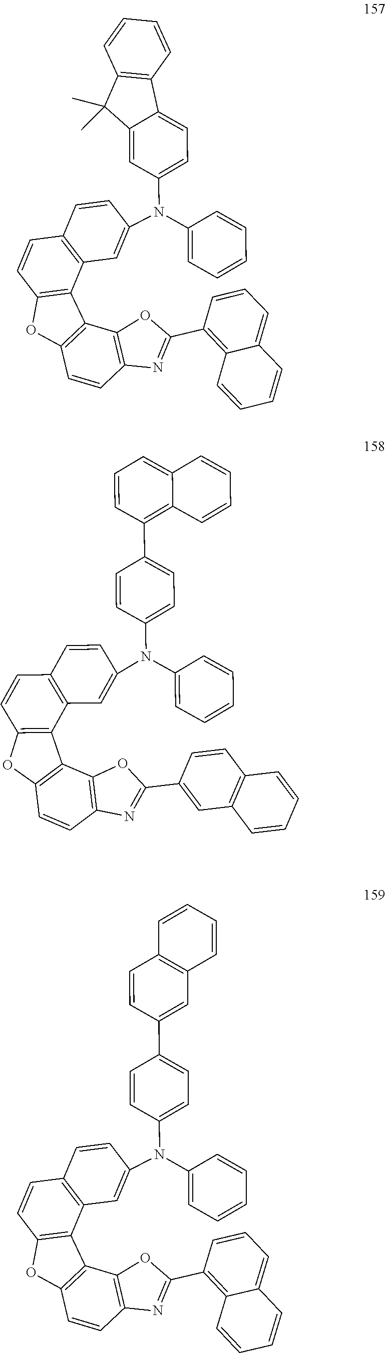 [SMILES: CC1(C)c2ccccc2-c2ccc(N(c3ccccc3)c3ccc4ccc5oc6ccc7nc(-c8cccc9ccccc89)oc7c6c5c4c3)cc21 ; c1ccc(N(c2ccc(-c3cccc4ccccc34)cc2)c2ccc3ccc4oc5ccc6nc(-c7ccc8ccccc8c7)oc6c5c4c3c2)cc1 ; c1ccc(N(c2ccc(-c3ccc4ccccc4c3)cc2)c2ccc3ccc4oc5ccc6nc(-c7cccc8ccccc78)oc6c5c4c3c2)cc1]

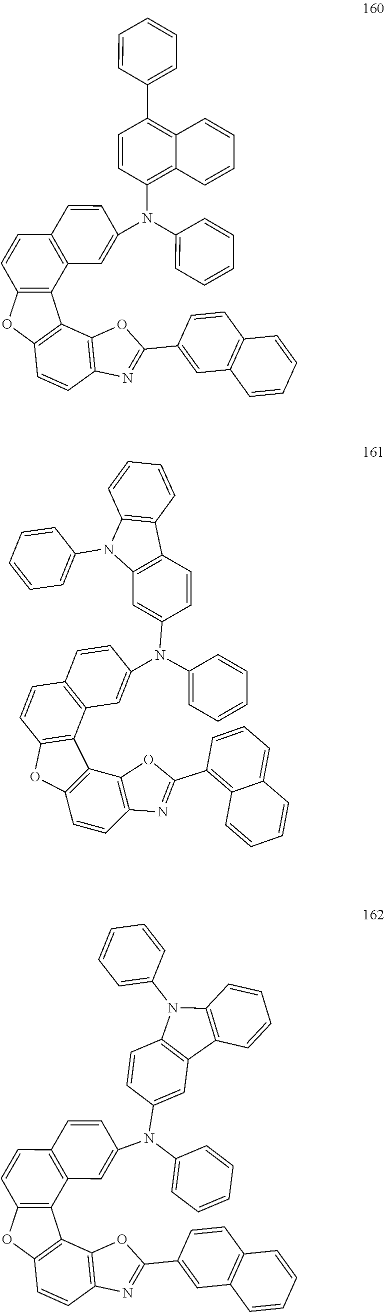 [SMILES: c1ccc(-c2ccc(N(c3ccccc3)c3ccc4ccc5oc6ccc7nc(-c8ccc9ccccc9c8)oc7c6c5c4c3)c3ccccc23)cc1 ; c1ccc(N(c2ccc3ccc4oc5ccc6nc(-c7cccc8ccccc78)oc6c5c4c3c2)c2ccc3c4ccccc4n(-c4ccccc4)c3c2)cc1 ; c1ccc(N(c2ccc3ccc4oc5ccc6nc(-c7ccc8ccccc8c7)oc6c5c4c3c2)c2ccc3c(c2)c2ccccc2n3-c2ccccc2)cc1]

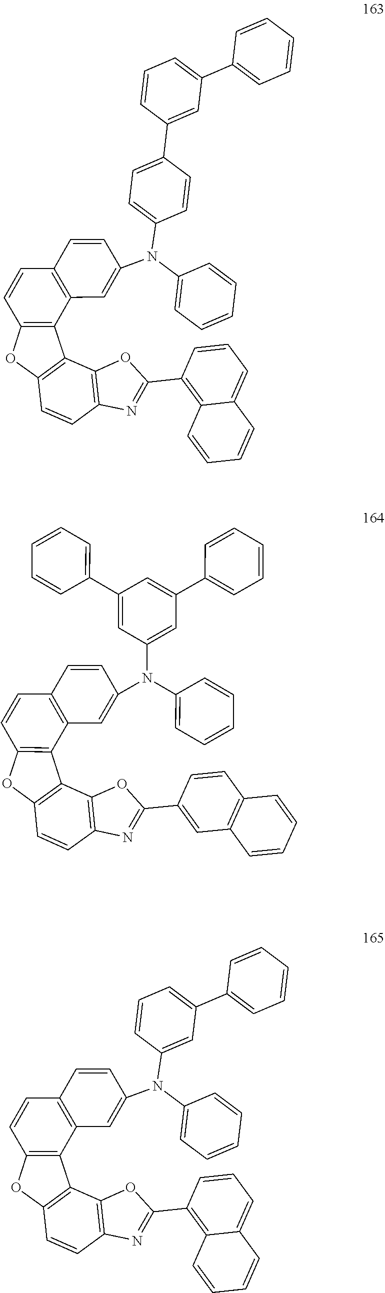 [SMILES: c1ccc(-c2cccc(-c3ccc(N(c4ccccc4)c4ccc5ccc6oc7ccc8nc(-c9cccc%10ccccc9%10)oc8c7c6c5c4)cc3)c2)cc1 ; c1ccc(-c2cc(-c3ccccc3)cc(N(c3ccccc3)c3ccc4ccc5oc6ccc7nc(-c8ccc9ccccc9c8)oc7c6c5c4c3)c2)cc1 ; c1ccc(-c2cccc(N(c3ccccc3)c3ccc4ccc5oc6ccc7nc(-c8cccc9ccccc89)oc7c6c5c4c3)c2)cc1]

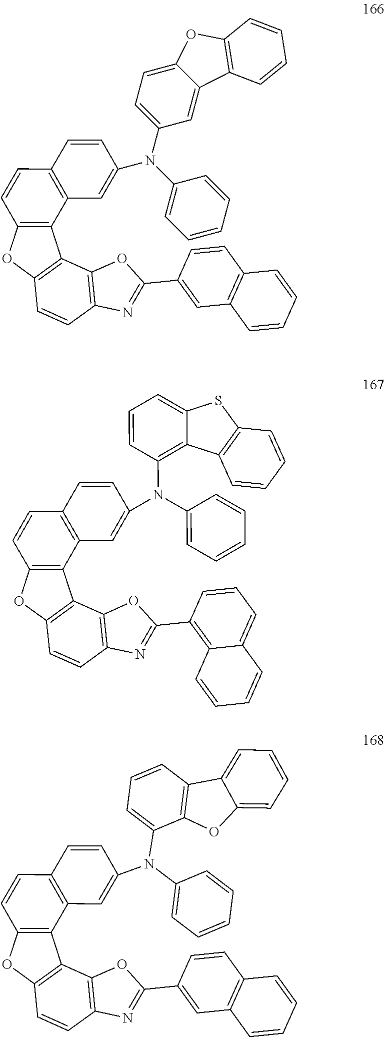 [SMILES: c1ccc(N(c2ccc3oc4ccccc4c3c2)c2ccc3ccc4oc5ccc6nc(-c7ccc8ccccc8c7)oc6c5c4c3c2)cc1 ; c1ccc(N(c2ccc3ccc4oc5ccc6nc(-c7cccc8ccccc78)oc6c5c4c3c2)c2cccc3sc4ccccc4c23)cc1 ; c1ccc(N(c2ccc3ccc4oc5ccc6nc(-c7ccc8ccccc8c7)oc6c5c4c3c2)c2cccc3c2oc2ccccc23)cc1]

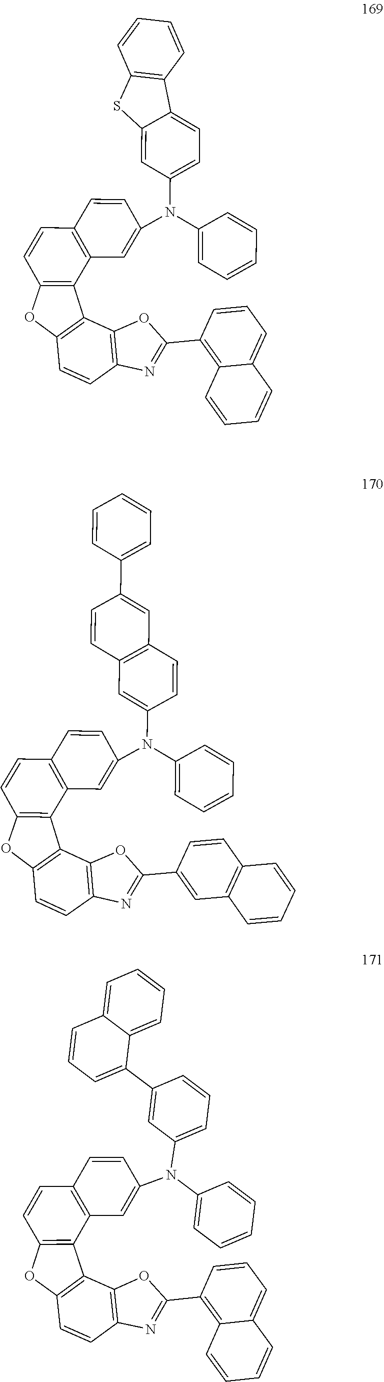 [SMILES: c1ccc(N(c2ccc3c(c2)sc2ccccc23)c2ccc3ccc4oc5ccc6nc(-c7cccc8ccccc78)oc6c5c4c3c2)cc1 ; c1ccc(-c2ccc3cc(N(c4ccccc4)c4ccc5ccc6oc7ccc8nc(-c9ccc%10ccccc%10c9)oc8c7c6c5c4)ccc3c2)cc1 ; c1ccc(N(c2cccc(-c3cccc4ccccc34)c2)c2ccc3ccc4oc5ccc6nc(-c7cccc8ccccc78)oc6c5c4c3c2)cc1]

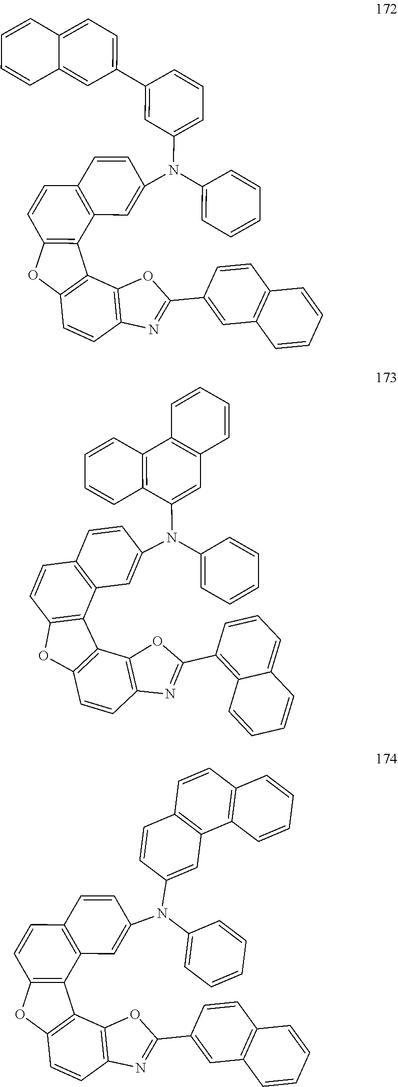

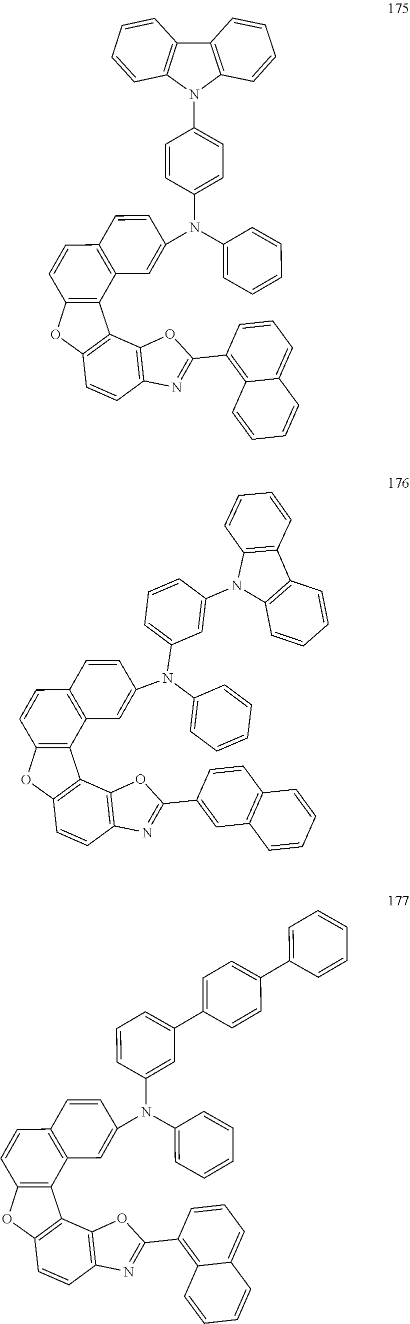 [SMILES: c1ccc(N(c2ccc(-n3c4ccccc4c4ccccc43)cc2)c2ccc3ccc4oc5ccc6nc(-c7cccc8ccccc78)oc6c5c4c3c2)cc1 ; c1ccc(N(c2cccc(-n3c4ccccc4c4ccccc43)c2)c2ccc3ccc4oc5ccc6nc(-c7ccc8ccccc8c7)oc6c5c4c3c2)cc1 ; c1ccc(-c2ccc(-c3cccc(N(c4ccccc4)c4ccc5ccc6oc7ccc8nc(-c9cccc%10ccccc9%10)oc8c7c6c5c4)c3)cc2)cc1]

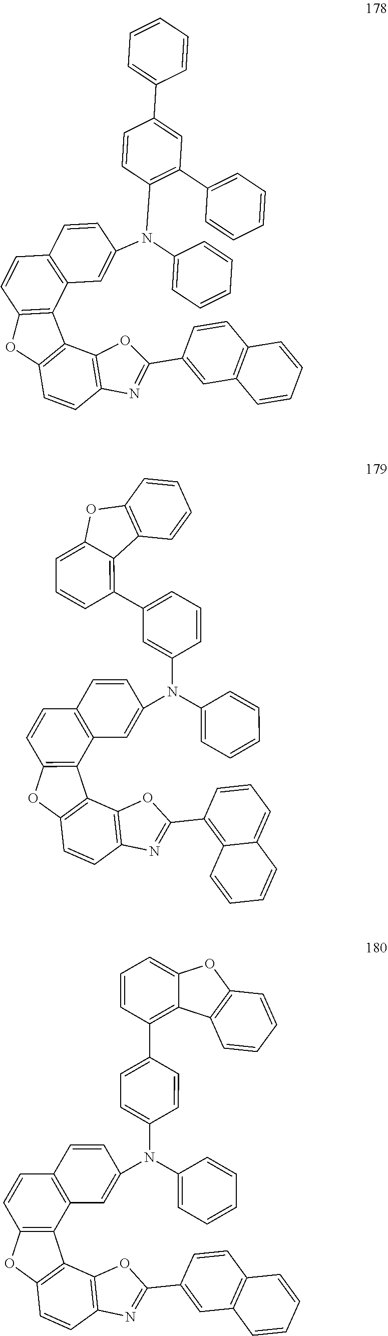 [SMILES: c1ccc(-c2ccc(N(c3ccccc3)c3ccc4ccc5oc6ccc7nc(-c8ccc9ccccc9c8)oc7c6c5c4c3)c(-c3ccccc3)c2)cc1 ; c1ccc(N(c2cccc(-c3cccc4oc5ccccc5c34)c2)c2ccc3ccc4oc5ccc6nc(-c7cccc8ccccc78)oc6c5c4c3c2)cc1 ; c1ccc(N(c2ccc(-c3cccc4oc5ccccc5c34)cc2)c2ccc3ccc4oc5ccc6nc(-c7ccc8ccccc8c7)oc6c5c4c3c2)cc1]

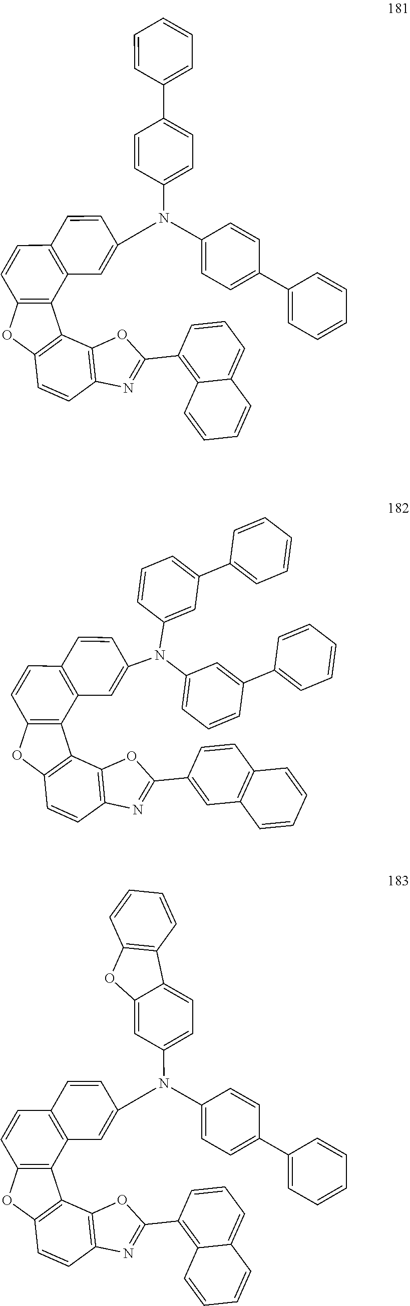 [SMILES: c1ccc(-c2ccc(N(c3ccc(-c4ccccc4)cc3)c3ccc4ccc5oc6ccc7nc(-c8cccc9ccccc89)oc7c6c5c4c3)cc2)cc1 ; c1ccc(-c2cccc(N(c3cccc(-c4ccccc4)c3)c3ccc4ccc5oc6ccc7nc(-c8ccc9ccccc9c8)oc7c6c5c4c3)c2)cc1 ; c1ccc(-c2ccc(N(c3ccc4c(c3)oc3ccccc34)c3ccc4ccc5oc6ccc7nc(-c8cccc9ccccc89)oc7c6c5c4c3)cc2)cc1]

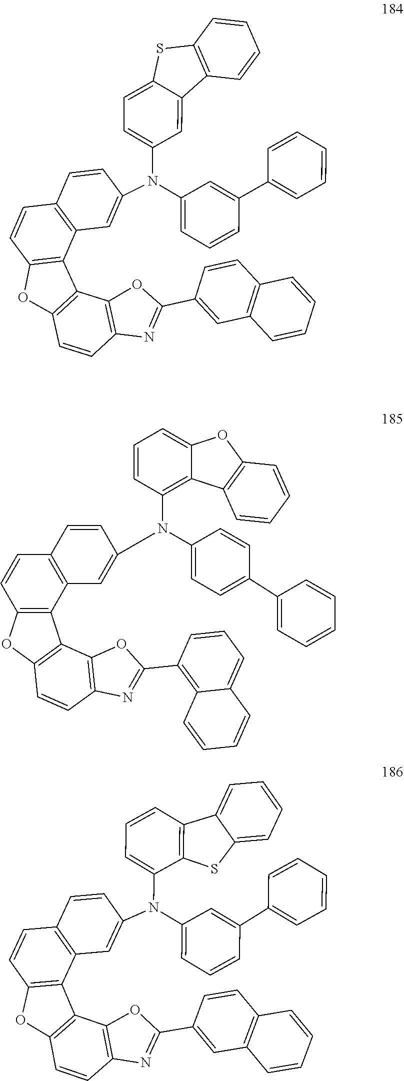

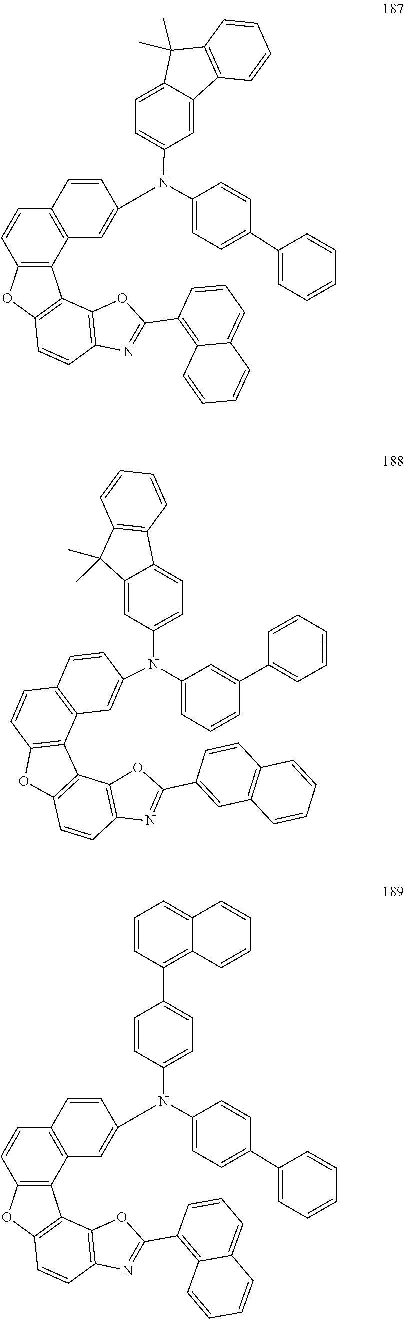 [SMILES: CC1(C)c2ccccc2-c2cc(N(c3ccc(-c4ccccc4)cc3)c3ccc4ccc5oc6ccc7nc(-c8cccc9ccccc89)oc7c6c5c4c3)ccc21 ; CC1(C)c2ccccc2-c2ccc(N(c3cccc(-c4ccccc4)c3)c3ccc4ccc5oc6ccc7nc(-c8ccc9ccccc9c8)oc7c6c5c4c3)cc21 ; c1ccc(-c2ccc(N(c3ccc(-c4cccc5ccccc45)cc3)c3ccc4ccc5oc6ccc7nc(-c8cccc9ccccc89)oc7c6c5c4c3)cc2)cc1]

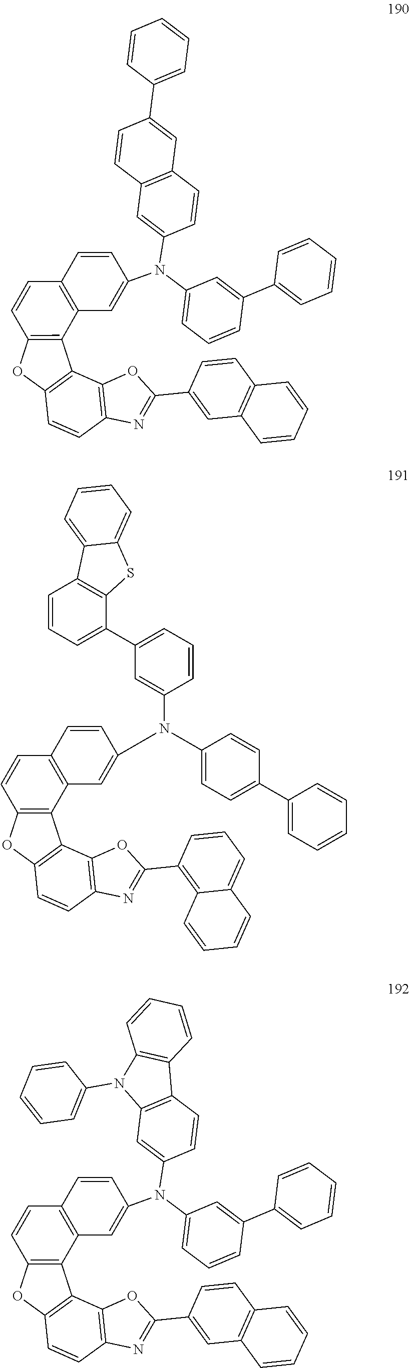 [SMILES: c1ccc(-c2cccc(N(c3ccc4cc(-c5ccccc5)ccc4c3)c3ccc4ccc5oc6ccc7nc(-c8ccc9ccccc9c8)oc7c6c5c4c3)c2)cc1 ; c1ccc(-c2ccc(N(c3cccc(-c4cccc5c4sc4ccccc45)c3)c3ccc4ccc5oc6ccc7nc(-c8cccc9ccccc89)oc7c6c5c4c3)cc2)cc1 ; c1ccc(-c2cccc(N(c3ccc4ccc5oc6ccc7nc(-c8ccc9ccccc9c8)oc7c6c5c4c3)c3ccc4c5ccccc5n(-c5ccccc5)c4c3)c2)cc1]

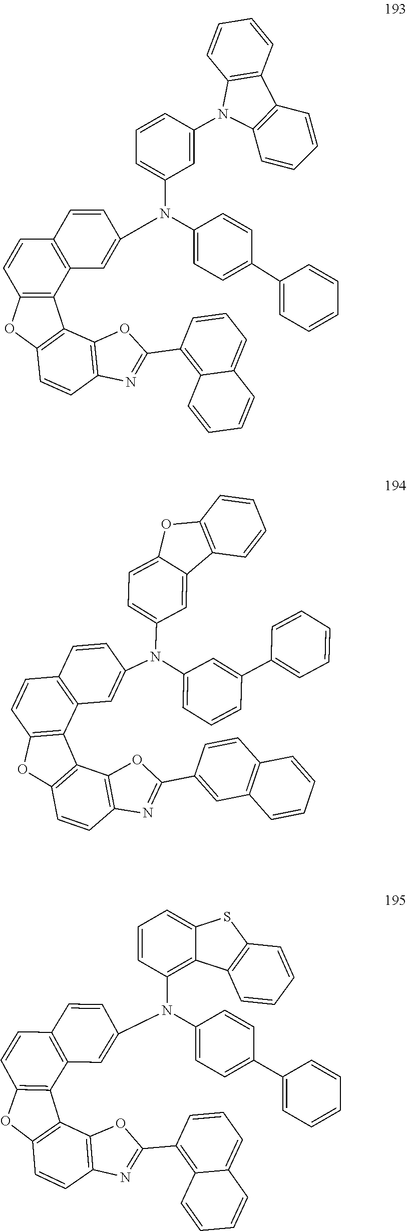 [SMILES: c1ccc(-c2ccc(N(c3cccc(-n4c5ccccc5c5ccccc54)c3)c3ccc4ccc5oc6ccc7nc(-c8cccc9ccccc89)oc7c6c5c4c3)cc2)cc1 ; c1ccc(-c2cccc(N(c3ccc4oc5ccccc5c4c3)c3ccc4ccc5oc6ccc7nc(-c8ccc9ccccc9c8)oc7c6c5c4c3)c2)cc1 ; c1ccc(-c2ccc(N(c3ccc4ccc5oc6ccc7nc(-c8cccc9ccccc89)oc7c6c5c4c3)c3cccc4sc5ccccc5c34)cc2)cc1]

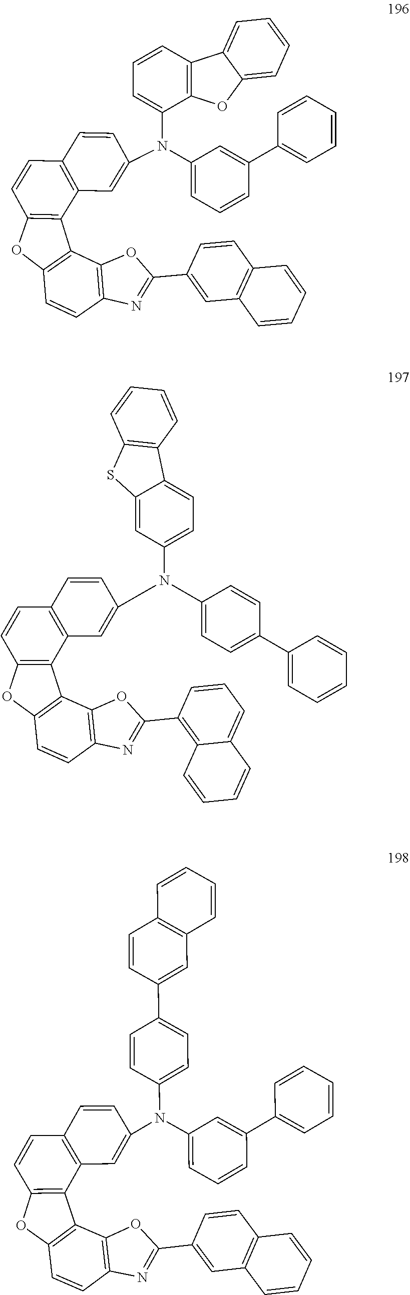 [SMILES: c1ccc(-c2cccc(N(c3ccc4ccc5oc6ccc7nc(-c8ccc9ccccc9c8)oc7c6c5c4c3)c3cccc4c3oc3ccccc34)c2)cc1 ; c1ccc(-c2ccc(N(c3ccc4c(c3)sc3ccccc34)c3ccc4ccc5oc6ccc7nc(-c8cccc9ccccc89)oc7c6c5c4c3)cc2)cc1 ; c1ccc(-c2cccc(N(c3ccc(-c4ccc5ccccc5c4)cc3)c3ccc4ccc5oc6ccc7nc(-c8ccc9ccccc9c8)oc7c6c5c4c3)c2)cc1]

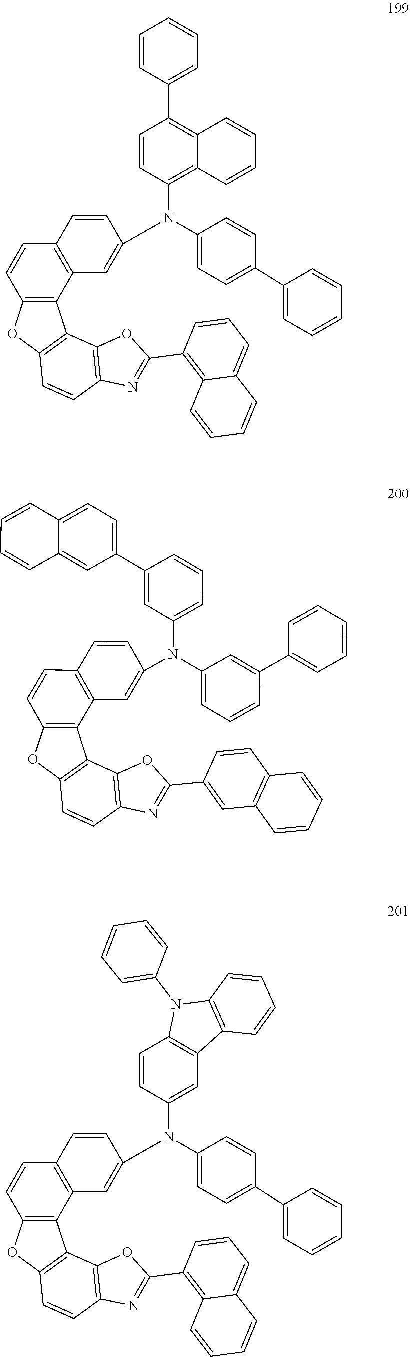 [SMILES: c1ccc(-c2ccc(N(c3ccc4ccc5oc6ccc7nc(-c8cccc9ccccc89)oc7c6c5c4c3)c3ccc(-c4ccccc4)c4ccccc34)cc2)cc1 ; c1ccc(-c2cccc(N(c3cccc(-c4ccc5ccccc5c4)c3)c3ccc4ccc5oc6ccc7nc(-c8ccc9ccccc9c8)oc7c6c5c4c3)c2)cc1 ; c1ccc(-c2ccc(N(c3ccc4ccc5oc6ccc7nc(-c8cccc9ccccc89)oc7c6c5c4c3)c3ccc4c(c3)c3ccccc3n4-c3ccccc3)cc2)cc1]

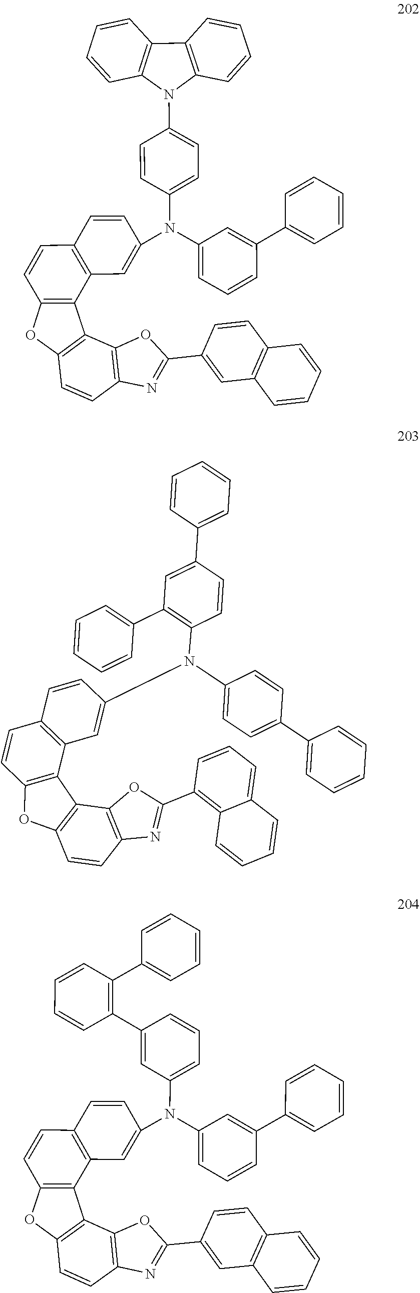 [SMILES: c1ccc(-c2cccc(N(c3ccc(-n4c5ccccc5c5ccccc54)cc3)c3ccc4ccc5oc6ccc7nc(-c8ccc9ccccc9c8)oc7c6c5c4c3)c2)cc1 ; c1ccc(-c2ccc(N(c3ccc4ccc5oc6ccc7nc(-c8cccc9ccccc89)oc7c6c5c4c3)c3ccc(-c4ccccc4)cc3-c3ccccc3)cc2)cc1 ; c1ccc(-c2cccc(N(c3cccc(-c4ccccc4-c4ccccc4)c3)c3ccc4ccc5oc6ccc7nc(-c8ccc9ccccc9c8)oc7c6c5c4c3)c2)cc1]

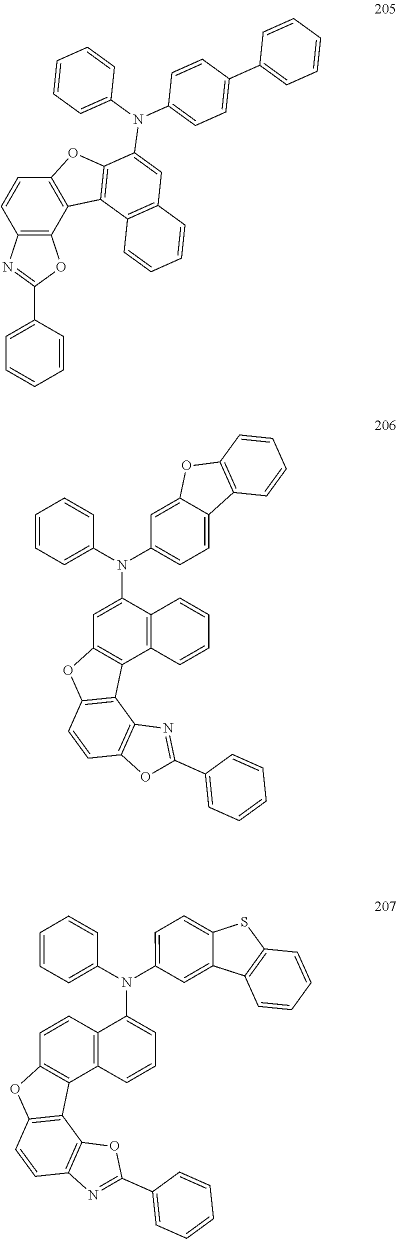 [SMILES: c1ccc(-c2ccc(N(c3ccccc3)c3cc4ccccc4c4c3oc3ccc5nc(-c6ccccc6)oc5c34)cc2)cc1 ; c1ccc(-c2nc3c(ccc4oc5cc(N(c6ccccc6)c6ccc7c(c6)oc6ccccc67)c6ccccc6c5c43)o2)cc1 ; c1ccc(-c2nc3ccc4oc5ccc6c(N(c7ccccc7)c7ccc8sc9ccccc9c8c7)cccc6c5c4c3o2)cc1]

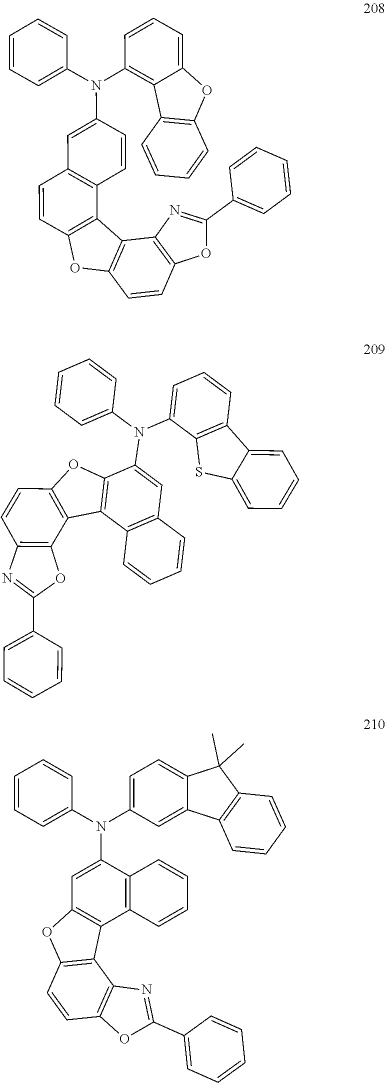 [SMILES: c1ccc(-c2nc3c(ccc4oc5ccc6cc(N(c7ccccc7)c7cccc8oc9ccccc9c78)ccc6c5c43)o2)cc1 ; c1ccc(-c2nc3ccc4oc5c(N(c6ccccc6)c6cccc7c6sc6ccccc67)cc6ccccc6c5c4c3o2)cc1 ; CC1(C)c2ccccc2-c2cc(N(c3ccccc3)c3cc4oc5ccc6oc(-c7ccccc7)nc6c5c4c4ccccc34)ccc21]

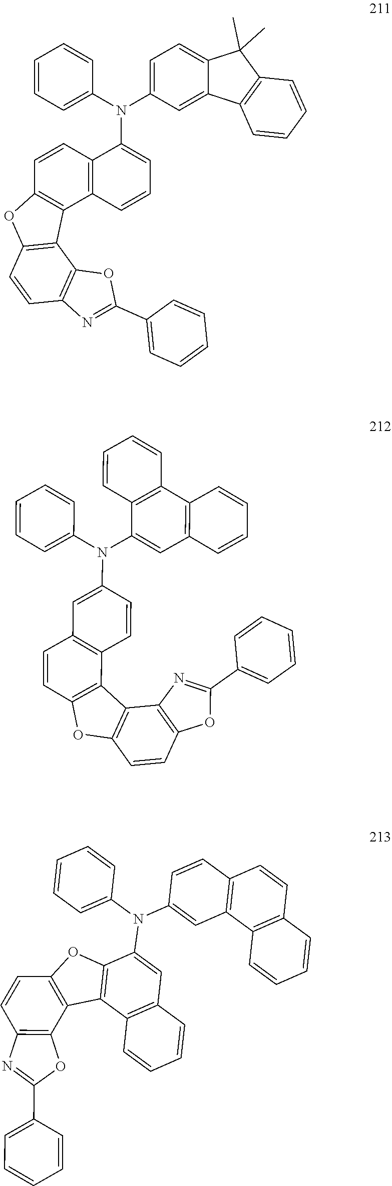 [SMILES: CC1(C)c2ccccc2-c2cc(N(c3ccccc3)c3cccc4c3ccc3oc5ccc6nc(-c7ccccc7)oc6c5c34)ccc21 ; c1ccc(-c2nc3c(ccc4oc5ccc6cc(N(c7ccccc7)c7cc8ccccc8c8ccccc78)ccc6c5c43)o2)cc1 ; c1ccc(-c2nc3ccc4oc5c(N(c6ccccc6)c6ccc7ccc8ccccc8c7c6)cc6ccccc6c5c4c3o2)cc1]

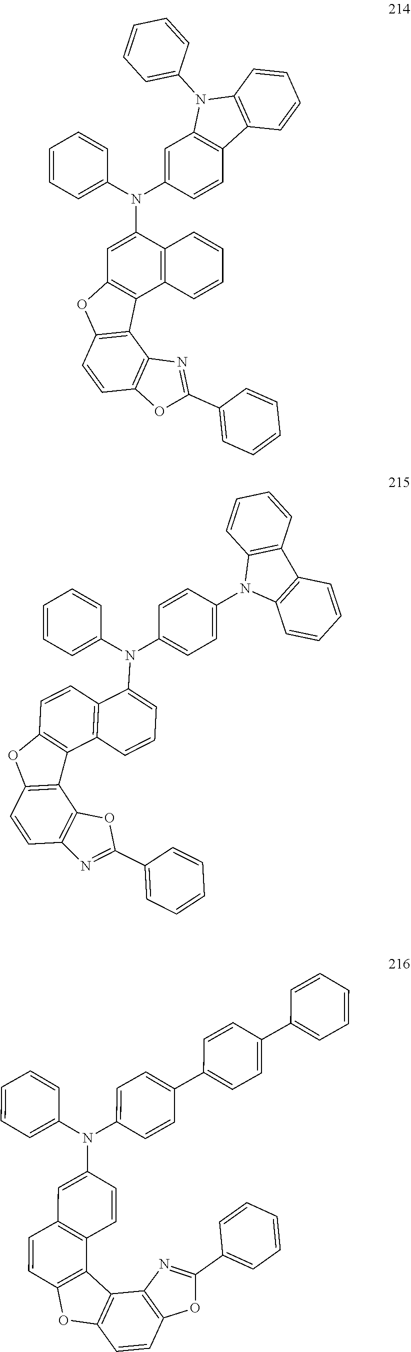

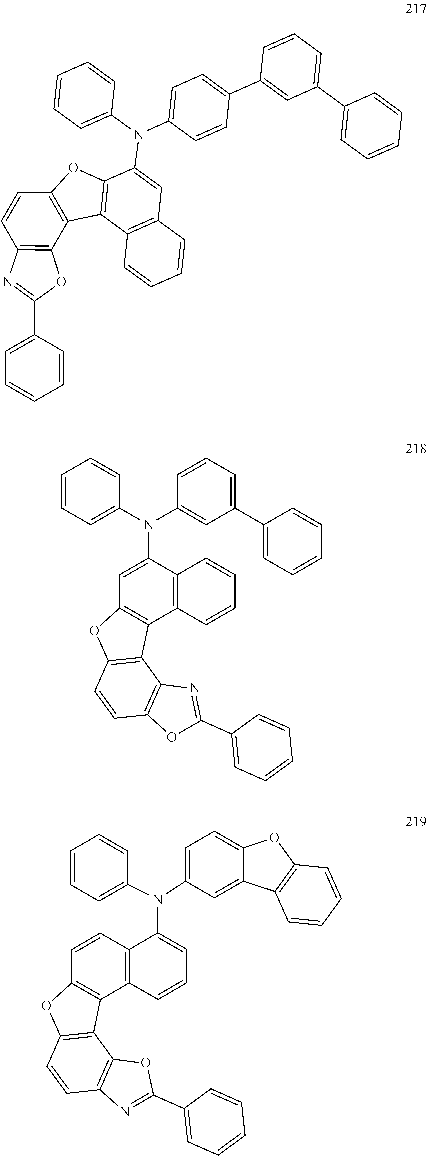 [SMILES: c1ccc(-c2cccc(-c3ccc(N(c4ccccc4)c4cc5ccccc5c5c4oc4ccc6nc(-c7ccccc7)oc6c45)cc3)c2)cc1 ; c1ccc(-c2cccc(N(c3ccccc3)c3cc4oc5ccc6oc(-c7ccccc7)nc6c5c4c4ccccc34)c2)cc1 ; c1ccc(-c2nc3ccc4oc5ccc6c(N(c7ccccc7)c7ccc8oc9ccccc9c8c7)cccc6c5c4c3o2)cc1]

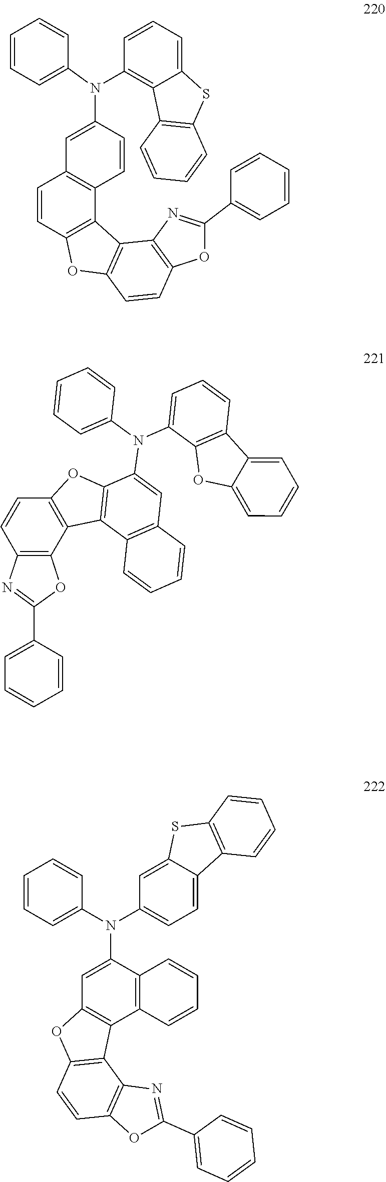 [SMILES: c1ccc(-c2nc3c(ccc4oc5ccc6cc(N(c7ccccc7)c7cccc8sc9ccccc9c78)ccc6c5c43)o2)cc1 ; c1ccc(-c2nc3ccc4oc5c(N(c6ccccc6)c6cccc7c6oc6ccccc67)cc6ccccc6c5c4c3o2)cc1 ; c1ccc(-c2nc3c(ccc4oc5cc(N(c6ccccc6)c6ccc7c(c6)sc6ccccc67)c6ccccc6c5c43)o2)cc1]

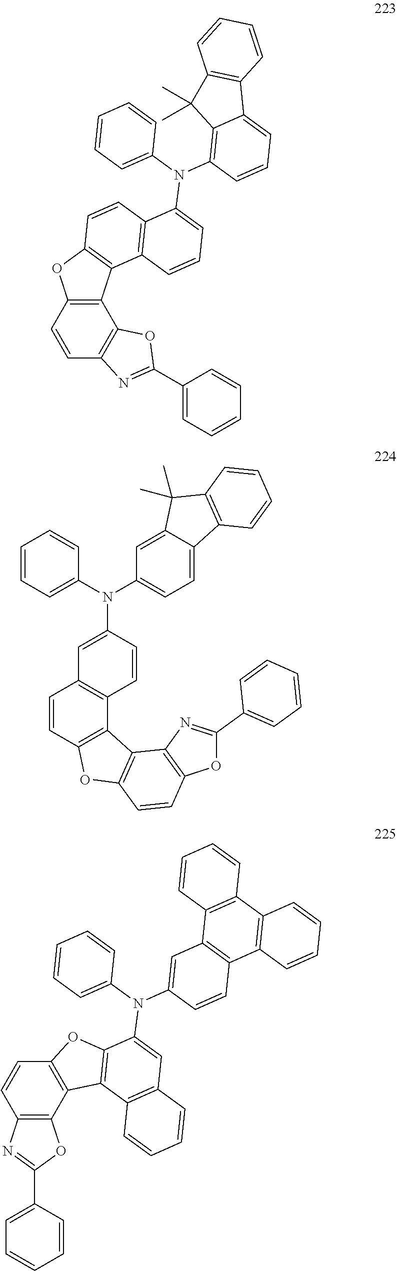

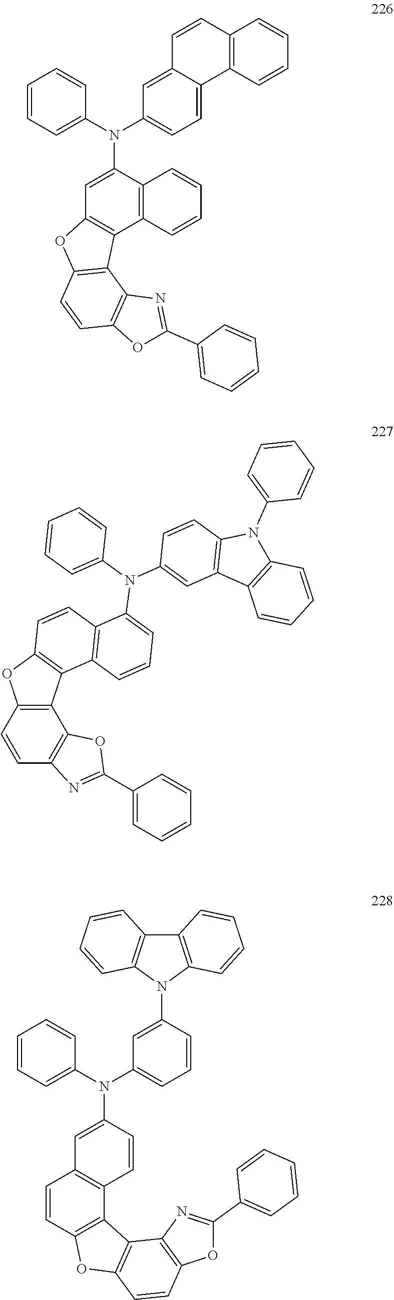 [SMILES: c1ccc(-c2nc3c(ccc4oc5cc(N(c6ccccc6)c6ccc7c(ccc8ccccc87)c6)c6ccccc6c5c43)o2)cc1 ; c1ccc(-c2nc3ccc4oc5ccc6c(N(c7ccccc7)c7ccc8c(c7)c7ccccc7n8-c7ccccc7)cccc6c5c4c3o2)cc1 ; c1ccc(-c2nc3c(ccc4oc5ccc6cc(N(c7ccccc7)c7cccc(-n8c9ccccc9c9ccccc98)c7)ccc6c5c43)o2)cc1]

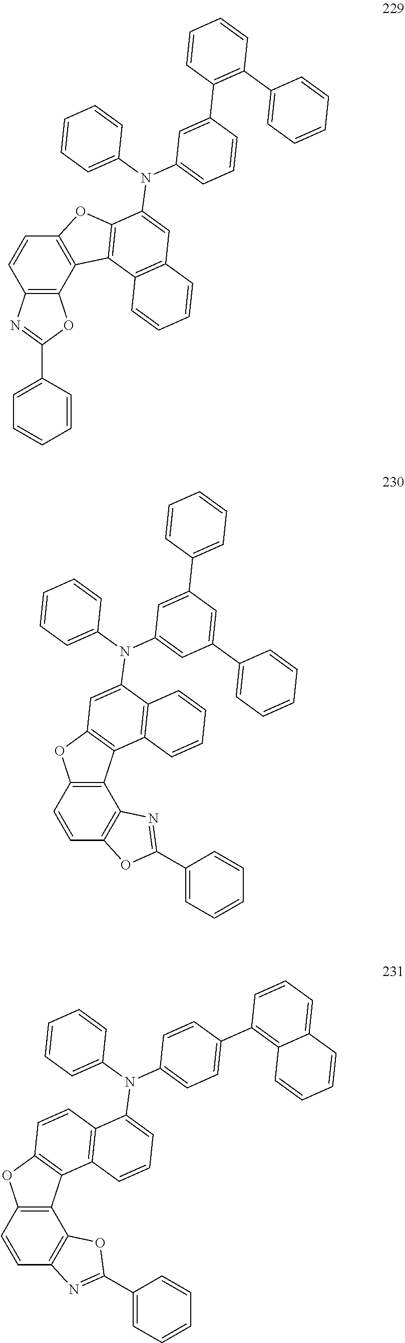 [SMILES: c1ccc(-c2nc3ccc4oc5c(N(c6ccccc6)c6cccc(-c7ccccc7-c7ccccc7)c6)cc6ccccc6c5c4c3o2)cc1 ; c1ccc(-c2cc(-c3ccccc3)cc(N(c3ccccc3)c3cc4oc5ccc6oc(-c7ccccc7)nc6c5c4c4ccccc34)c2)cc1 ; c1ccc(-c2nc3ccc4oc5ccc6c(N(c7ccccc7)c7ccc(-c8cccc9ccccc89)cc7)cccc6c5c4c3o2)cc1]

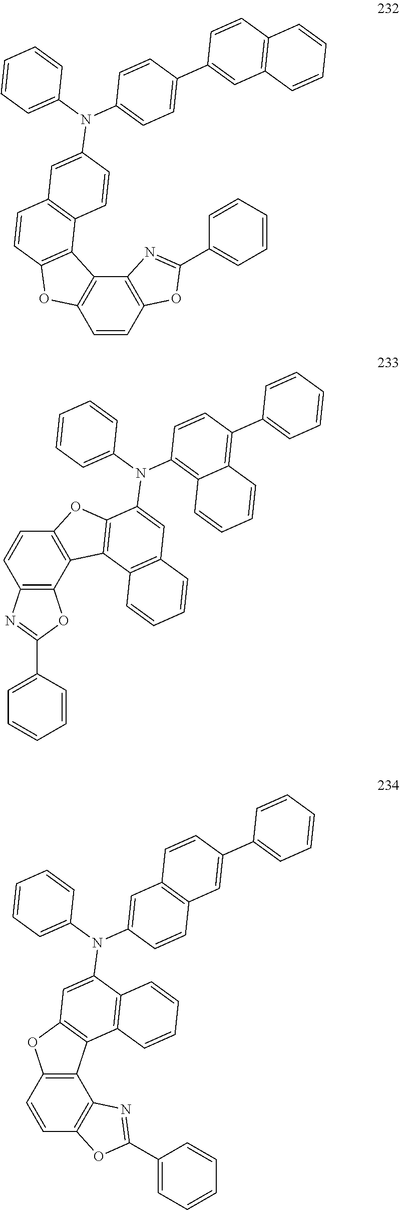 [SMILES: c1ccc(-c2nc3c(ccc4oc5ccc6cc(N(c7ccccc7)c7ccc(-c8ccc9ccccc9c8)cc7)ccc6c5c43)o2)cc1 ; c1ccc(-c2nc3ccc4oc5c(N(c6ccccc6)c6ccc(-c7ccccc7)c7ccccc67)cc6ccccc6c5c4c3o2)cc1 ; c1ccc(-c2ccc3cc(N(c4ccccc4)c4cc5oc6ccc7oc(-c8ccccc8)nc7c6c5c5ccccc45)ccc3c2)cc1]

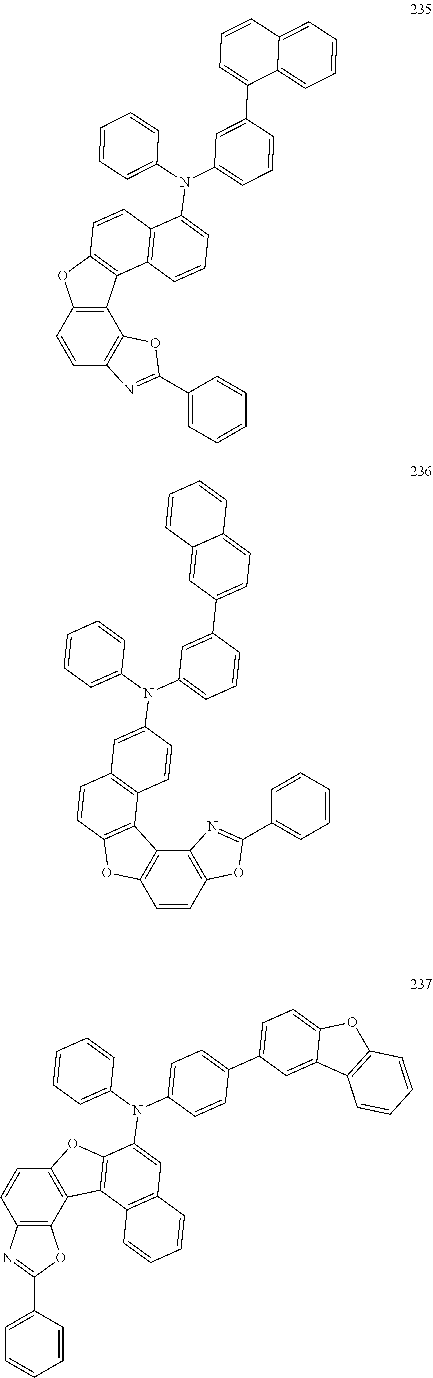

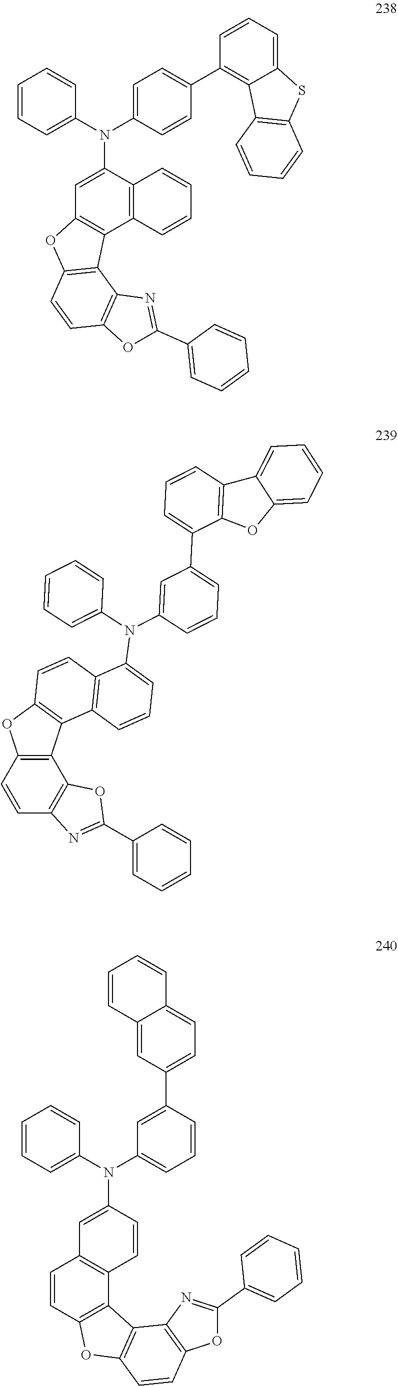 [SMILES: c1ccc(-c2nc3c(ccc4oc5cc(N(c6ccccc6)c6ccc(-c7cccc8sc9ccccc9c78)cc6)c6ccccc6c5c43)o2)cc1 ; c1ccc(-c2nc3ccc4oc5ccc6c(N(c7ccccc7)c7cccc(-c8cccc9c8oc8ccccc89)c7)cccc6c5c4c3o2)cc1 ; c1ccc(-c2nc3c(ccc4oc5ccc6cc(N(c7ccccc7)c7cccc(-c8ccc9ccccc9c8)c7)ccc6c5c43)o2)cc1]

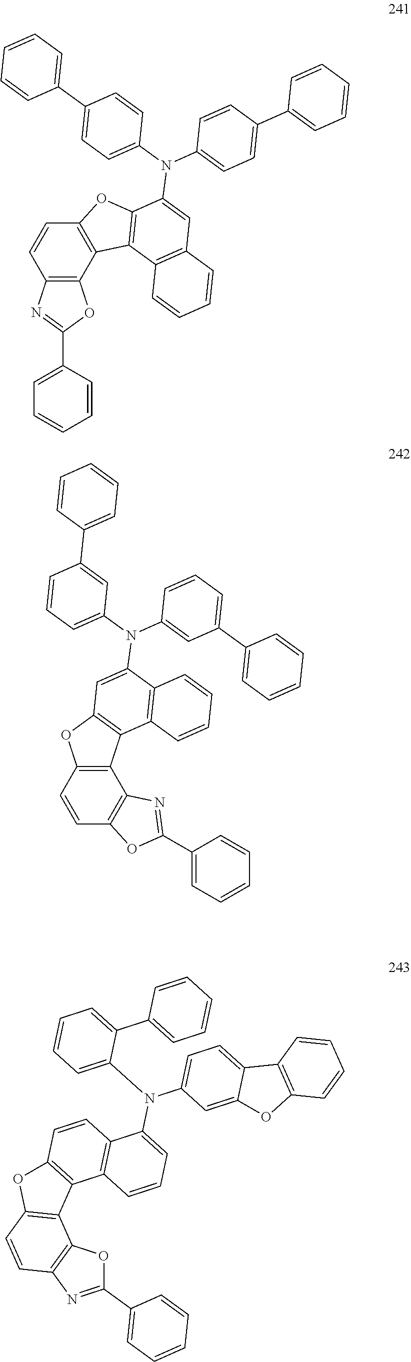

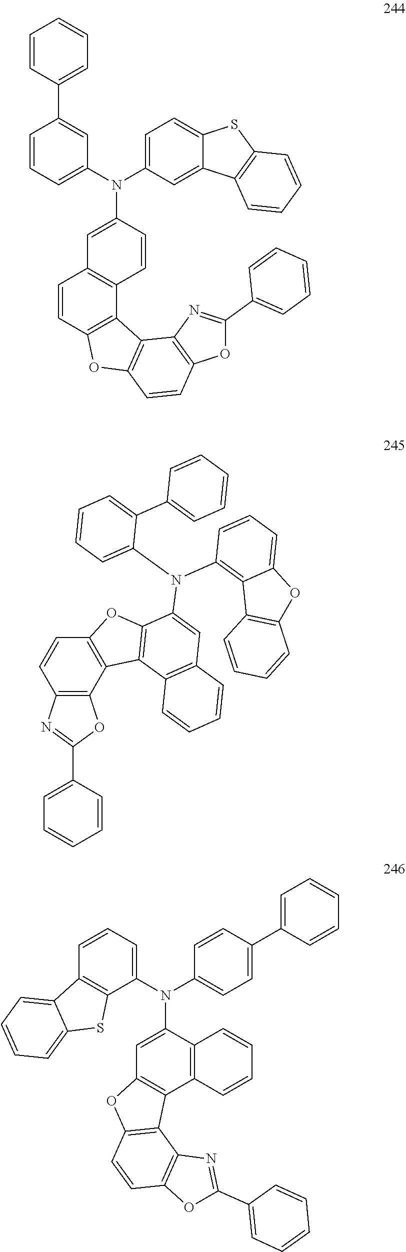 [SMILES: c1ccc(-c2cccc(N(c3ccc4c(ccc5oc6ccc7oc(-c8ccccc8)nc7c6c54)c3)c3ccc4sc5ccccc5c4c3)c2)cc1 ; c1ccc(-c2nc3ccc4oc5c(N(c6ccccc6-c6ccccc6)c6cccc7oc8ccccc8c67)cc6ccccc6c5c4c3o2)cc1 ; c1ccc(-c2ccc(N(c3cc4oc5ccc6oc(-c7ccccc7)nc6c5c4c4ccccc34)c3cccc4c3sc3ccccc34)cc2)cc1]

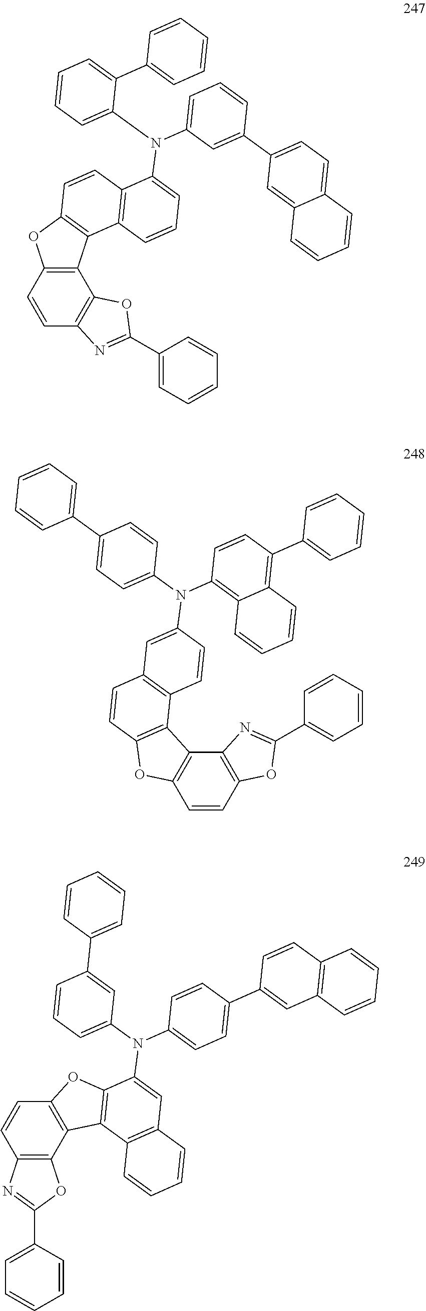 [SMILES: c1ccc(-c2nc3ccc4oc5ccc6c(N(c7cccc(-c8ccc9ccccc9c8)c7)c7ccccc7-c7ccccc7)cccc6c5c4c3o2)cc1 ; c1ccc(-c2ccc(N(c3ccc4c(ccc5oc6ccc7oc(-c8ccccc8)nc7c6c54)c3)c3ccc(-c4ccccc4)c4ccccc34)cc2)cc1 ; c1ccc(-c2cccc(N(c3ccc(-c4ccc5ccccc5c4)cc3)c3cc4ccccc4c4c3oc3ccc5nc(-c6ccccc6)oc5c34)c2)cc1]

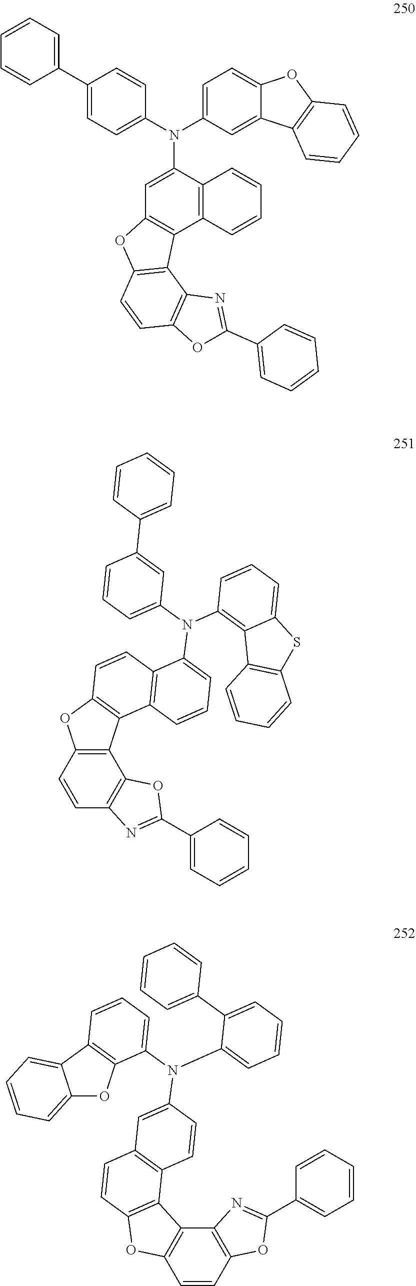 [SMILES: c1ccc(-c2ccc(N(c3ccc4oc5ccccc5c4c3)c3cc4oc5ccc6oc(-c7ccccc7)nc6c5c4c4ccccc34)cc2)cc1 ; c1ccc(-c2cccc(N(c3cccc4c3ccc3oc5ccc6nc(-c7ccccc7)oc6c5c34)c3cccc4sc5ccccc5c34)c2)cc1 ; c1ccc(-c2nc3c(ccc4oc5ccc6cc(N(c7ccccc7-c7ccccc7)c7cccc8c7oc7ccccc78)ccc6c5c43)o2)cc1]

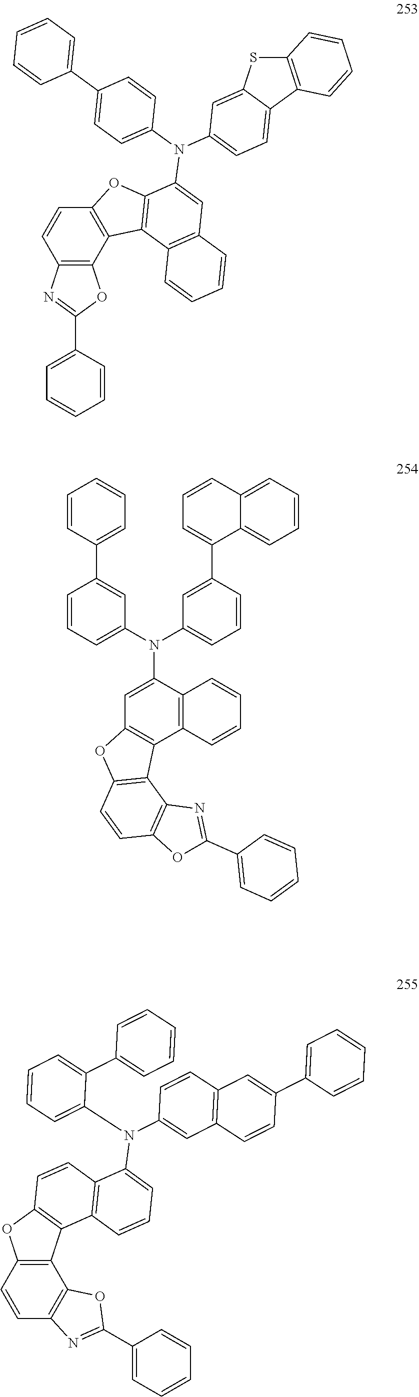 [SMILES: c1ccc(-c2ccc(N(c3ccc4c(c3)sc3ccccc34)c3cc4ccccc4c4c3oc3ccc5nc(-c6ccccc6)oc5c34)cc2)cc1 ; c1ccc(-c2cccc(N(c3cccc(-c4cccc5ccccc45)c3)c3cc4oc5ccc6oc(-c7ccccc7)nc6c5c4c4ccccc34)c2)cc1 ; c1ccc(-c2ccc3cc(N(c4ccccc4-c4ccccc4)c4cccc5c4ccc4oc6ccc7nc(-c8ccccc8)oc7c6c45)ccc3c2)cc1]

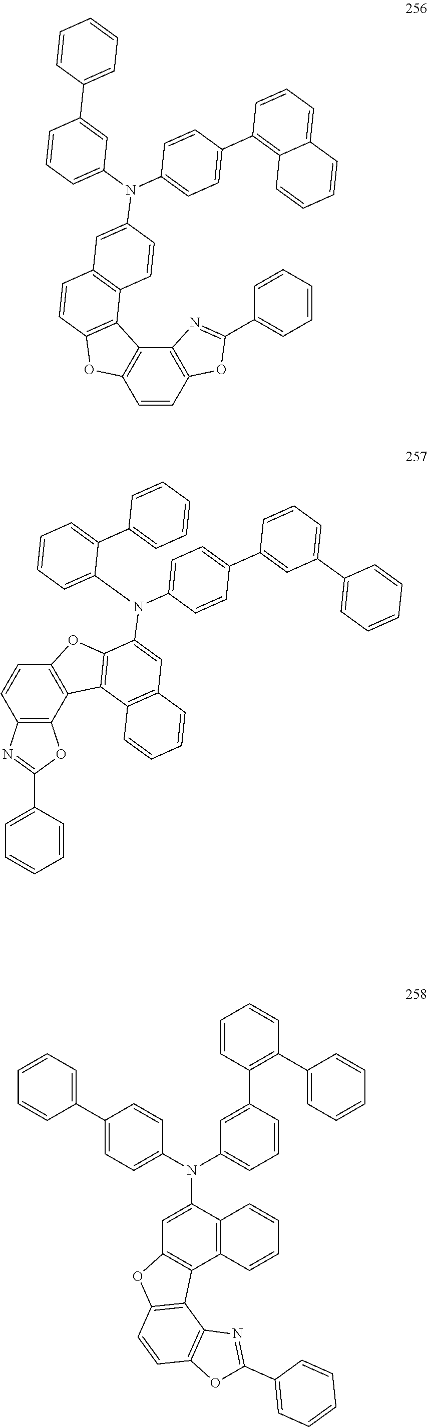 [SMILES: c1ccc(-c2cccc(N(c3ccc(-c4cccc5ccccc45)cc3)c3ccc4c(ccc5oc6ccc7oc(-c8ccccc8)nc7c6c54)c3)c2)cc1 ; c1ccc(-c2cccc(-c3ccc(N(c4ccccc4-c4ccccc4)c4cc5ccccc5c5c4oc4ccc6nc(-c7ccccc7)oc6c45)cc3)c2)cc1 ; c1ccc(-c2ccc(N(c3cccc(-c4ccccc4-c4ccccc4)c3)c3cc4oc5ccc6oc(-c7ccccc7)nc6c5c4c4ccccc34)cc2)cc1]

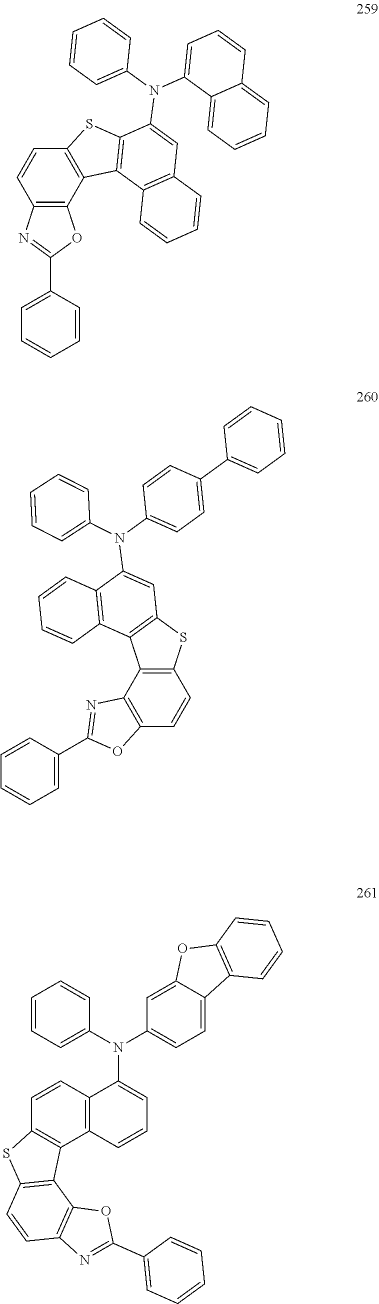 [SMILES: c1ccc(-c2nc3ccc4sc5c(N(c6ccccc6)c6cccc7ccccc67)cc6ccccc6c5c4c3o2)cc1 ; c1ccc(-c2ccc(N(c3ccccc3)c3cc4sc5ccc6oc(-c7ccccc7)nc6c5c4c4ccccc34)cc2)cc1 ; c1ccc(-c2nc3ccc4sc5ccc6c(N(c7ccccc7)c7ccc8c(c7)oc7ccccc78)cccc6c5c4c3o2)cc1]

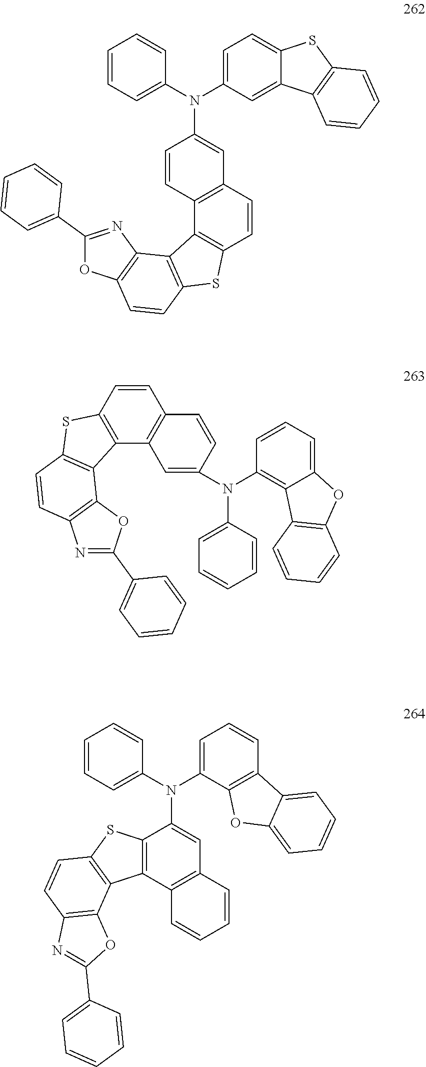 [SMILES: c1ccc(-c2nc3c(ccc4sc5ccc6cc(N(c7ccccc7)c7ccc8sc9ccccc9c8c7)ccc6c5c43)o2)cc1 ; c1ccc(-c2nc3ccc4sc5ccc6ccc(N(c7ccccc7)c7cccc8oc9ccccc9c78)cc6c5c4c3o2)cc1 ; c1ccc(-c2nc3ccc4sc5c(N(c6ccccc6)c6cccc7c6oc6ccccc67)cc6ccccc6c5c4c3o2)cc1]

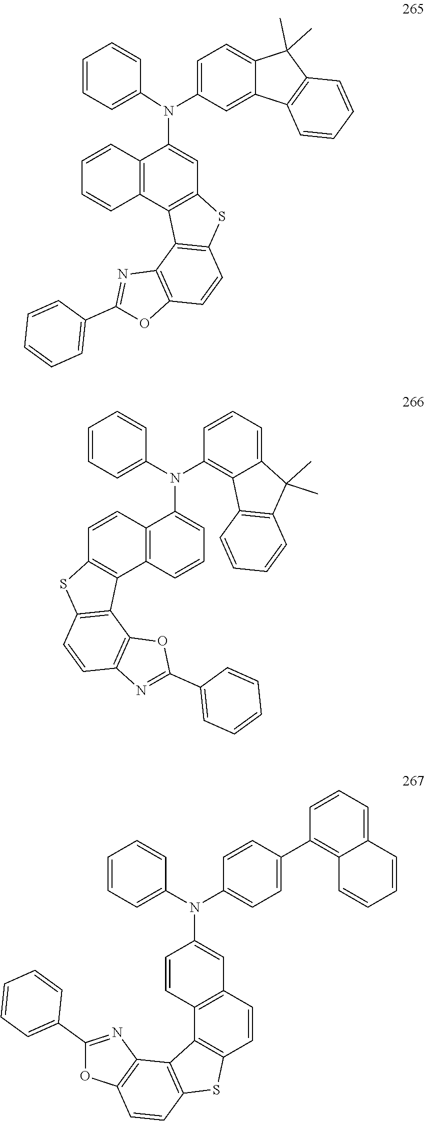 [SMILES: CC1(C)c2ccccc2-c2cc(N(c3ccccc3)c3cc4sc5ccc6oc(-c7ccccc7)nc6c5c4c4ccccc34)ccc21 ; CC1(C)c2ccccc2-c2c(N(c3ccccc3)c3cccc4c3ccc3sc5ccc6nc(-c7ccccc7)oc6c5c34)cccc21 ; c1ccc(-c2nc3c(ccc4sc5ccc6cc(N(c7ccccc7)c7ccc(-c8cccc9ccccc89)cc7)ccc6c5c43)o2)cc1]

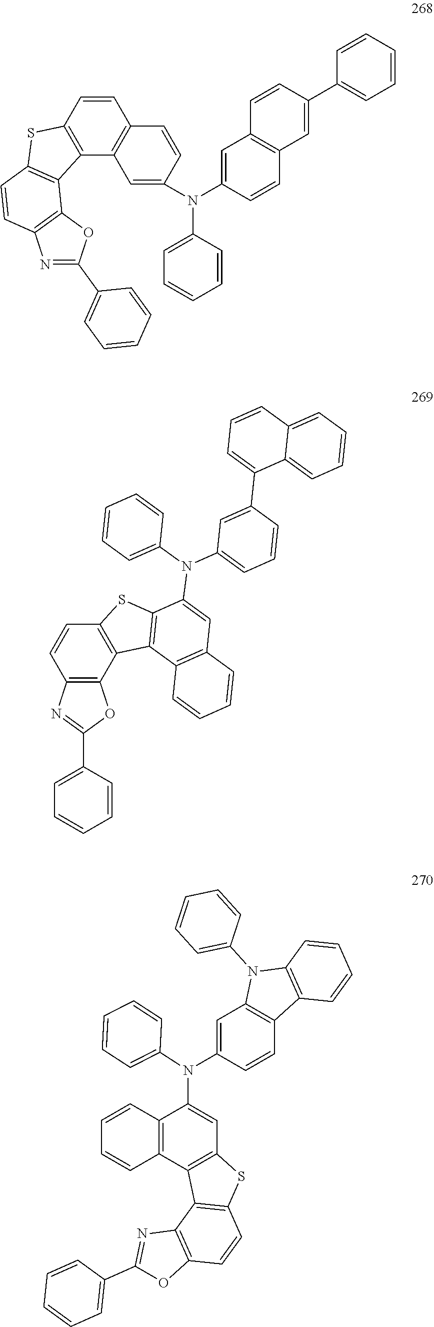 [SMILES: c1ccc(-c2ccc3cc(N(c4ccccc4)c4ccc5ccc6sc7ccc8nc(-c9ccccc9)oc8c7c6c5c4)ccc3c2)cc1 ; c1ccc(-c2nc3ccc4sc5c(N(c6ccccc6)c6cccc(-c7cccc8ccccc78)c6)cc6ccccc6c5c4c3o2)cc1 ; c1ccc(-c2nc3c(ccc4sc5cc(N(c6ccccc6)c6ccc7c8ccccc8n(-c8ccccc8)c7c6)c6ccccc6c5c43)o2)cc1]

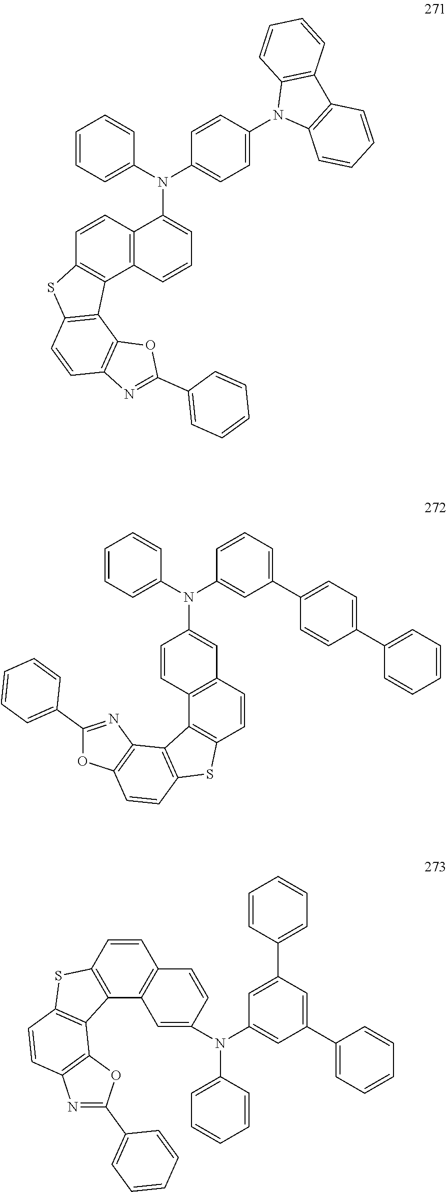 [SMILES: c1ccc(-c2nc3ccc4sc5ccc6c(N(c7ccccc7)c7ccc(-n8c9ccccc9c9ccccc98)cc7)cccc6c5c4c3o2)cc1 ; c1ccc(-c2ccc(-c3cccc(N(c4ccccc4)c4ccc5c(ccc6sc7ccc8oc(-c9ccccc9)nc8c7c65)c4)c3)cc2)cc1 ; c1ccc(-c2cc(-c3ccccc3)cc(N(c3ccccc3)c3ccc4ccc5sc6ccc7nc(-c8ccccc8)oc7c6c5c4c3)c2)cc1]

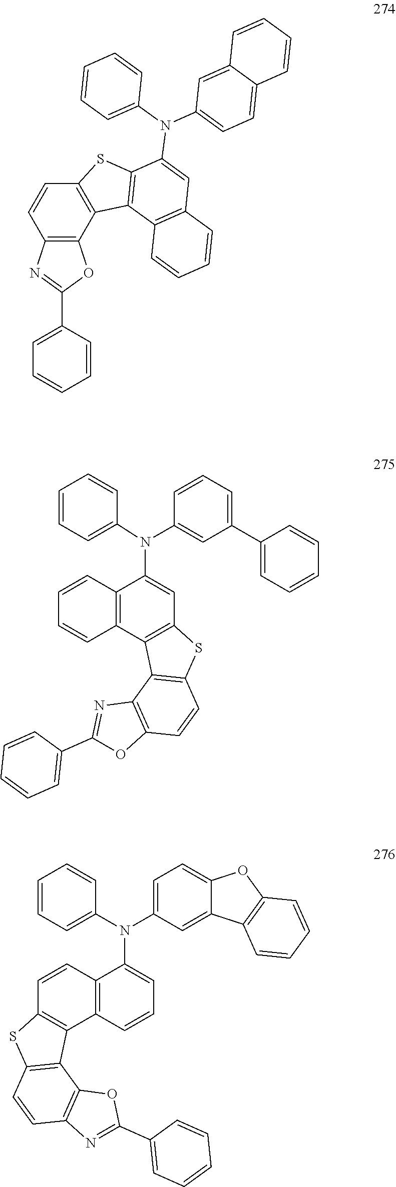 [SMILES: c1ccc(-c2nc3ccc4sc5c(N(c6ccccc6)c6ccc7ccccc7c6)cc6ccccc6c5c4c3o2)cc1 ; c1ccc(-c2cccc(N(c3ccccc3)c3cc4sc5ccc6oc(-c7ccccc7)nc6c5c4c4ccccc34)c2)cc1 ; c1ccc(-c2nc3ccc4sc5ccc6c(N(c7ccccc7)c7ccc8oc9ccccc9c8c7)cccc6c5c4c3o2)cc1]

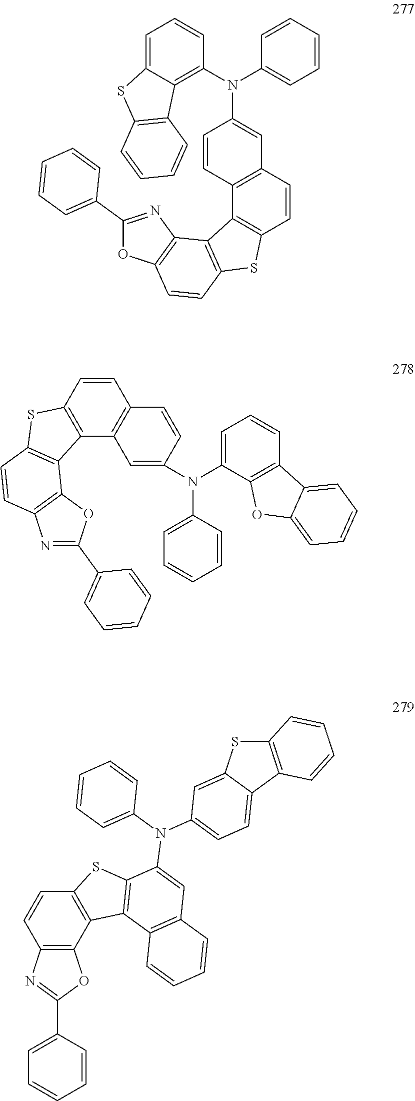 [SMILES: c1ccc(-c2nc3c(ccc4sc5ccc6cc(N(c7ccccc7)c7cccc8sc9ccccc9c78)ccc6c5c43)o2)cc1 ; c1ccc(-c2nc3ccc4sc5ccc6ccc(N(c7ccccc7)c7cccc8c7oc7ccccc78)cc6c5c4c3o2)cc1 ; c1ccc(-c2nc3ccc4sc5c(N(c6ccccc6)c6ccc7c(c6)sc6ccccc67)cc6ccccc6c5c4c3o2)cc1]

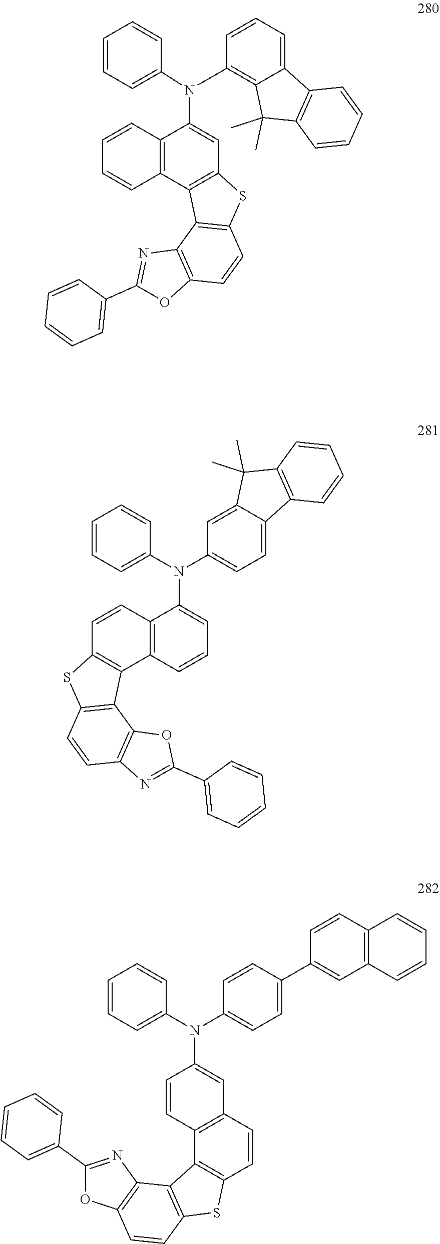 [SMILES: CC1(C)c2ccccc2-c2cccc(N(c3ccccc3)c3cc4sc5ccc6oc(-c7ccccc7)nc6c5c4c4ccccc34)c21 ; CC1(C)c2ccccc2-c2ccc(N(c3ccccc3)c3cccc4c3ccc3sc5ccc6nc(-c7ccccc7)oc6c5c34)cc21 ; c1ccc(-c2nc3c(ccc4sc5ccc6cc(N(c7ccccc7)c7ccc(-c8ccc9ccccc9c8)cc7)ccc6c5c43)o2)cc1]

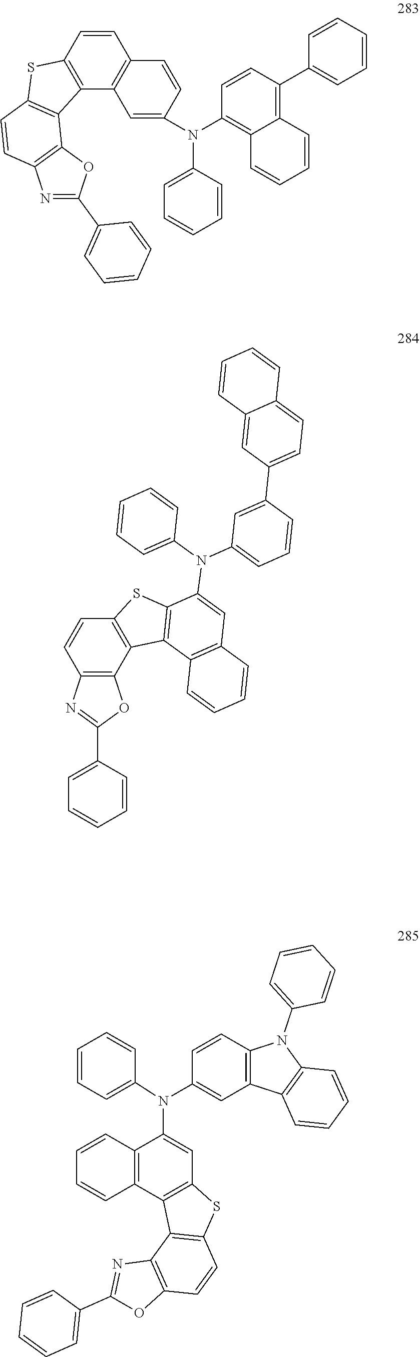 [SMILES: c1ccc(-c2nc3ccc4sc5ccc6ccc(N(c7ccccc7)c7ccc(-c8ccccc8)c8ccccc78)cc6c5c4c3o2)cc1 ; c1ccc(-c2nc3ccc4sc5c(N(c6ccccc6)c6cccc(-c7ccc8ccccc8c7)c6)cc6ccccc6c5c4c3o2)cc1 ; c1ccc(-c2nc3c(ccc4sc5cc(N(c6ccccc6)c6ccc7c(c6)c6ccccc6n7-c6ccccc6)c6ccccc6c5c43)o2)cc1]

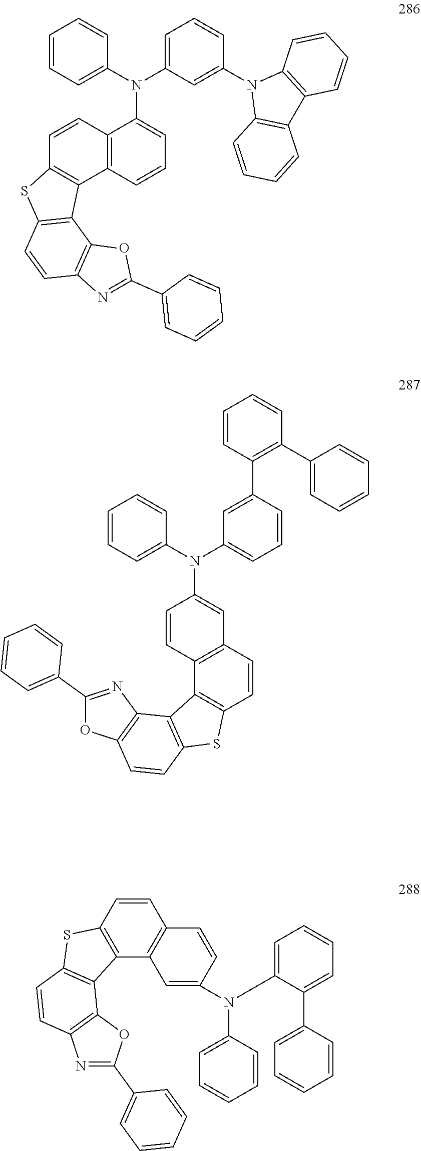 [SMILES: c1ccc(-c2nc3ccc4sc5ccc6c(N(c7ccccc7)c7cccc(-n8c9ccccc9c9ccccc98)c7)cccc6c5c4c3o2)cc1 ; c1ccc(-c2nc3c(ccc4sc5ccc6cc(N(c7ccccc7)c7cccc(-c8ccccc8-c8ccccc8)c7)ccc6c5c43)o2)cc1 ; c1ccc(-c2nc3ccc4sc5ccc6ccc(N(c7ccccc7)c7ccccc7-c7ccccc7)cc6c5c4c3o2)cc1]

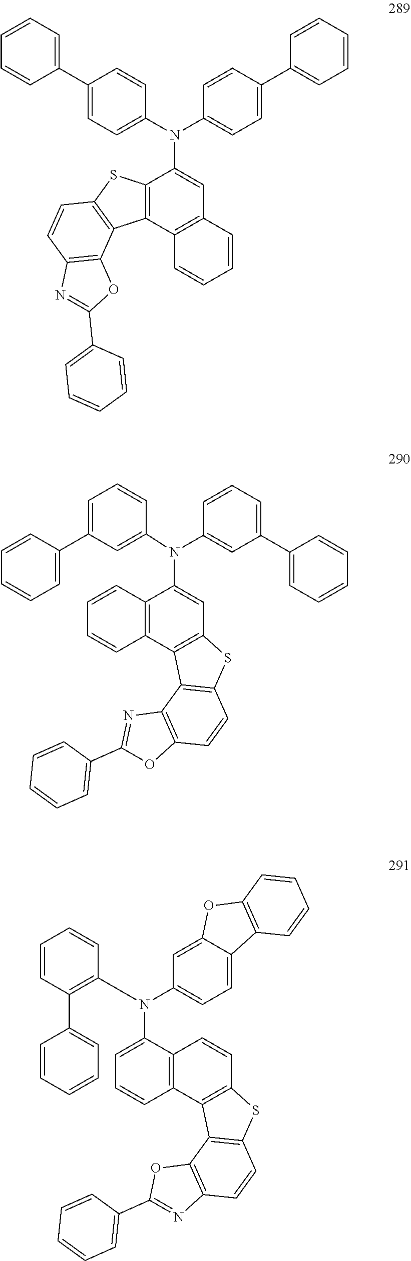 [SMILES: c1ccc(-c2ccc(N(c3ccc(-c4ccccc4)cc3)c3cc4ccccc4c4c3sc3ccc5nc(-c6ccccc6)oc5c34)cc2)cc1 ; c1ccc(-c2cccc(N(c3cccc(-c4ccccc4)c3)c3cc4sc5ccc6oc(-c7ccccc7)nc6c5c4c4ccccc34)c2)cc1 ; c1ccc(-c2nc3ccc4sc5ccc6c(N(c7ccc8c(c7)oc7ccccc78)c7ccccc7-c7ccccc7)cccc6c5c4c3o2)cc1]

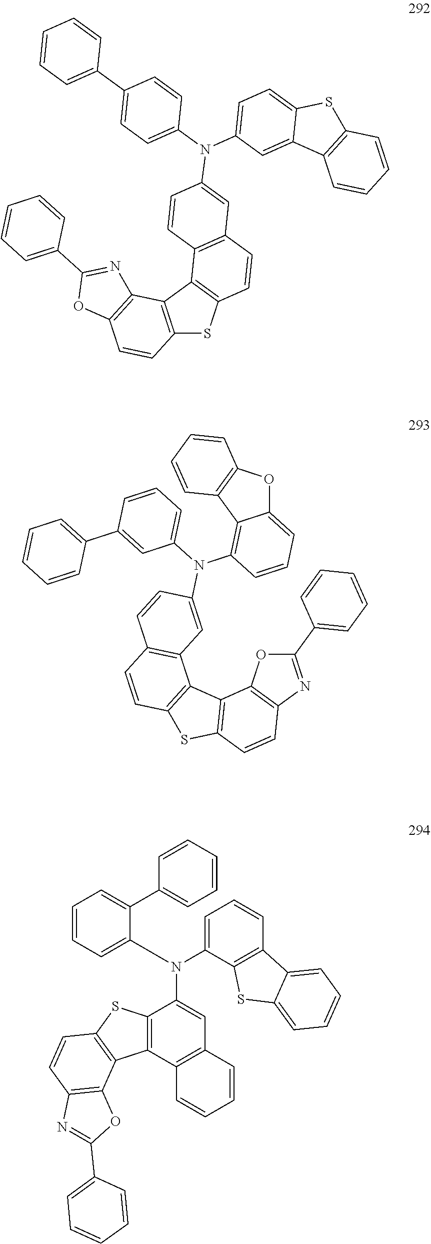 [SMILES: c1ccc(-c2ccc(N(c3ccc4c(ccc5sc6ccc7oc(-c8ccccc8)nc7c6c54)c3)c3ccc4sc5ccccc5c4c3)cc2)cc1 ; c1ccc(-c2cccc(N(c3ccc4ccc5sc6ccc7nc(-c8ccccc8)oc7c6c5c4c3)c3cccc4oc5ccccc5c34)c2)cc1 ; c1ccc(-c2nc3ccc4sc5c(N(c6ccccc6-c6ccccc6)c6cccc7c6sc6ccccc67)cc6ccccc6c5c4c3o2)cc1]

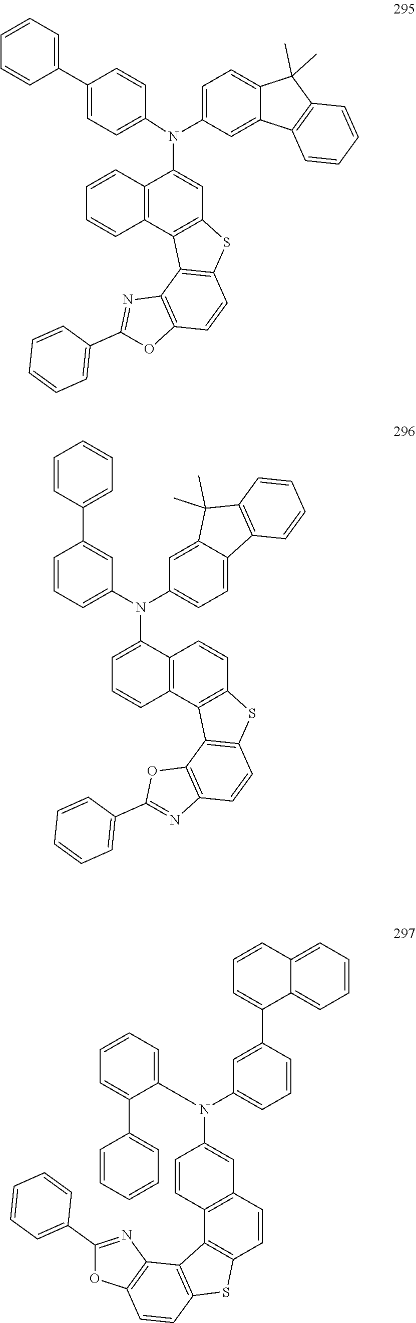 [SMILES: CC1(C)c2ccccc2-c2cc(N(c3ccc(-c4ccccc4)cc3)c3cc4sc5ccc6oc(-c7ccccc7)nc6c5c4c4ccccc34)ccc21 ; CC1(C)c2ccccc2-c2ccc(N(c3cccc(-c4ccccc4)c3)c3cccc4c3ccc3sc5ccc6nc(-c7ccccc7)oc6c5c34)cc21 ; c1ccc(-c2nc3c(ccc4sc5ccc6cc(N(c7cccc(-c8cccc9ccccc89)c7)c7ccccc7-c7ccccc7)ccc6c5c43)o2)cc1]

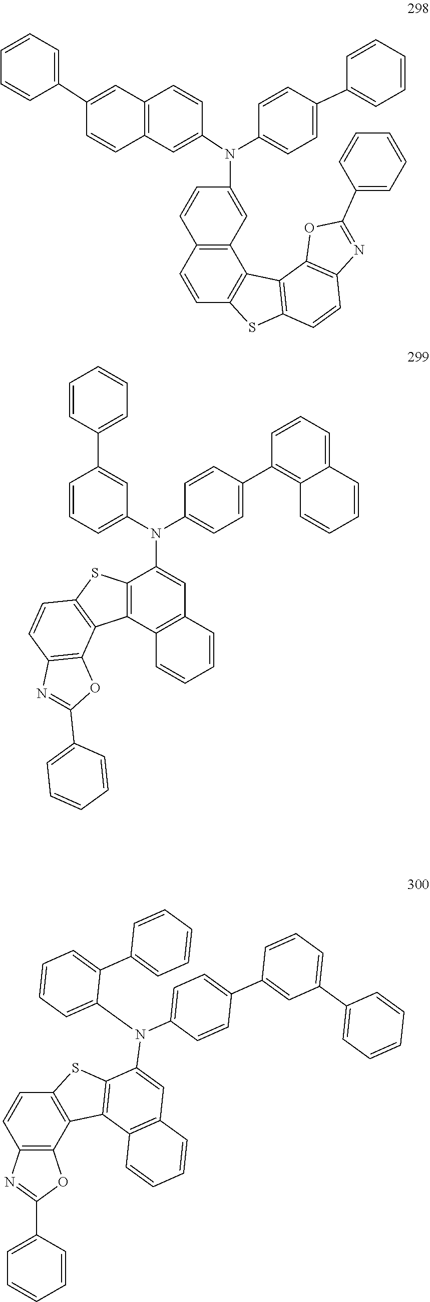 [SMILES: c1ccc(-c2ccc(N(c3ccc4cc(-c5ccccc5)ccc4c3)c3ccc4ccc5sc6ccc7nc(-c8ccccc8)oc7c6c5c4c3)cc2)cc1 ; c1ccc(-c2cccc(N(c3ccc(-c4cccc5ccccc45)cc3)c3cc4ccccc4c4c3sc3ccc5nc(-c6ccccc6)oc5c34)c2)cc1 ; c1ccc(-c2cccc(-c3ccc(N(c4ccccc4-c4ccccc4)c4cc5ccccc5c5c4sc4ccc6nc(-c7ccccc7)oc6c45)cc3)c2)cc1]

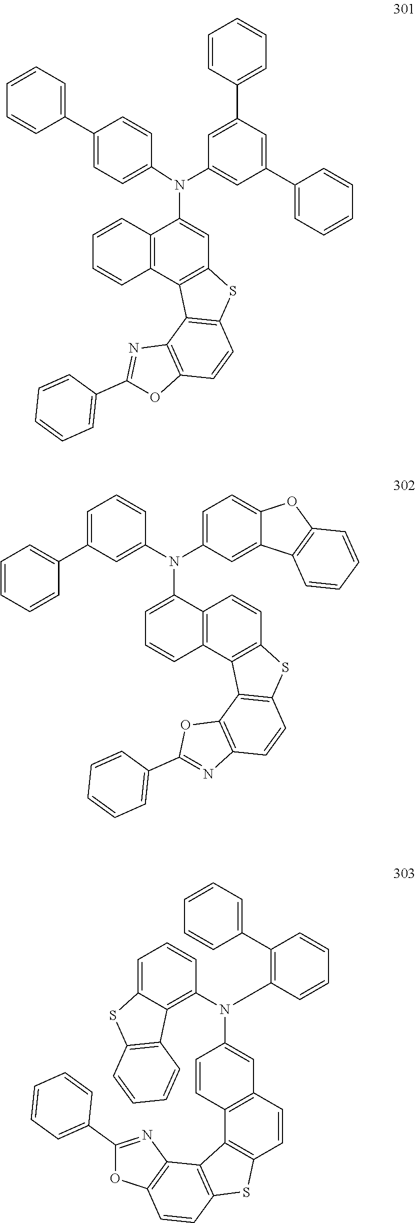 [SMILES: c1ccc(-c2ccc(N(c3cc(-c4ccccc4)cc(-c4ccccc4)c3)c3cc4sc5ccc6oc(-c7ccccc7)nc6c5c4c4ccccc34)cc2)cc1 ; c1ccc(-c2cccc(N(c3ccc4oc5ccccc5c4c3)c3cccc4c3ccc3sc5ccc6nc(-c7ccccc7)oc6c5c34)c2)cc1 ; c1ccc(-c2nc3c(ccc4sc5ccc6cc(N(c7ccccc7-c7ccccc7)c7cccc8sc9ccccc9c78)ccc6c5c43)o2)cc1]

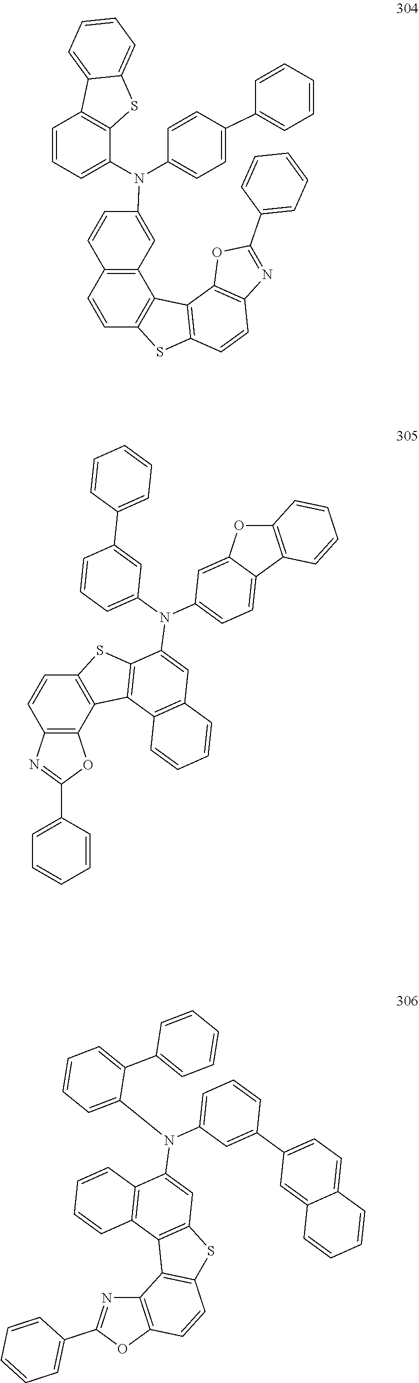 [SMILES: c1ccc(-c2ccc(N(c3ccc4ccc5sc6ccc7nc(-c8ccccc8)oc7c6c5c4c3)c3cccc4c3sc3ccccc34)cc2)cc1 ; c1ccc(-c2cccc(N(c3ccc4c(c3)oc3ccccc34)c3cc4ccccc4c4c3sc3ccc5nc(-c6ccccc6)oc5c34)c2)cc1 ; c1ccc(-c2nc3c(ccc4sc5cc(N(c6cccc(-c7ccc8ccccc8c7)c6)c6ccccc6-c6ccccc6)c6ccccc6c5c43)o2)cc1]

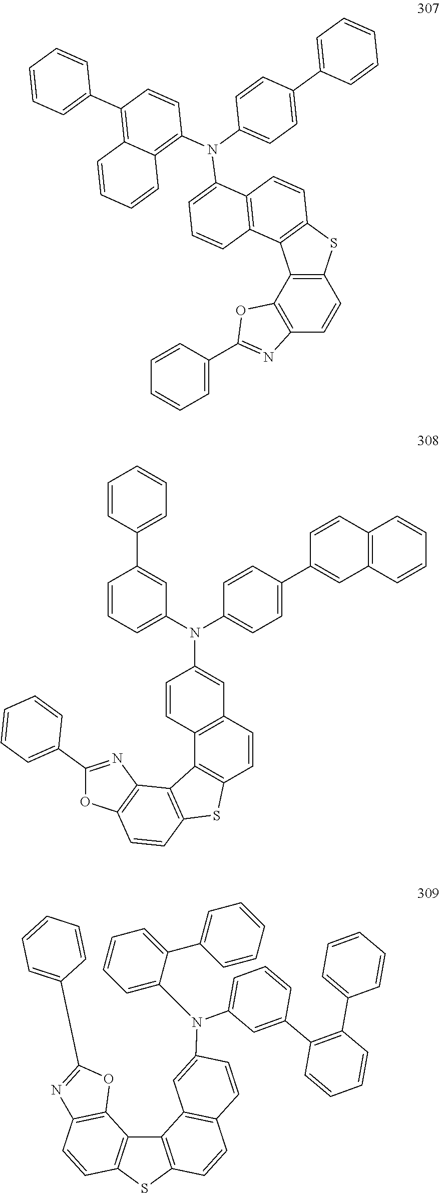 [SMILES: c1ccc(-c2ccc(N(c3ccc(-c4ccccc4)c4ccccc34)c3cccc4c3ccc3sc5ccc6nc(-c7ccccc7)oc6c5c34)cc2)cc1 ; c1ccc(-c2cccc(N(c3ccc(-c4ccc5ccccc5c4)cc3)c3ccc4c(ccc5sc6ccc7oc(-c8ccccc8)nc7c6c54)c3)c2)cc1 ; c1ccc(-c2nc3ccc4sc5ccc6ccc(N(c7cccc(-c8ccccc8-c8ccccc8)c7)c7ccccc7-c7ccccc7)cc6c5c4c3o2)cc1]

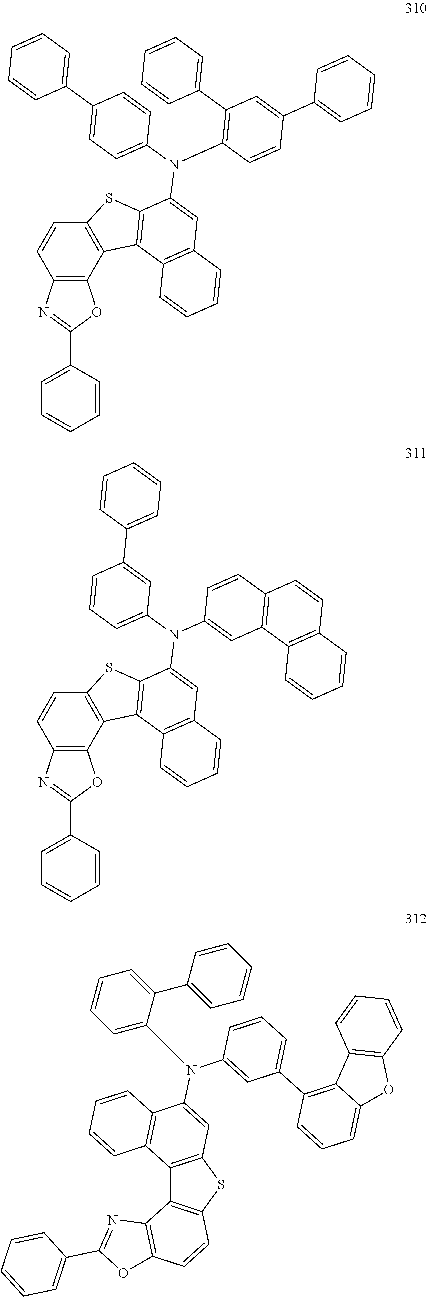 [SMILES: c1ccc(-c2ccc(N(c3ccc(-c4ccccc4)cc3-c3ccccc3)c3cc4ccccc4c4c3sc3ccc5nc(-c6ccccc6)oc5c34)cc2)cc1 ; c1ccc(-c2cccc(N(c3ccc4ccc5ccccc5c4c3)c3cc4ccccc4c4c3sc3ccc5nc(-c6ccccc6)oc5c34)c2)cc1 ; c1ccc(-c2nc3c(ccc4sc5cc(N(c6cccc(-c7cccc8oc9ccccc9c78)c6)c6ccccc6-c6ccccc6)c6ccccc6c5c43)o2)cc1]

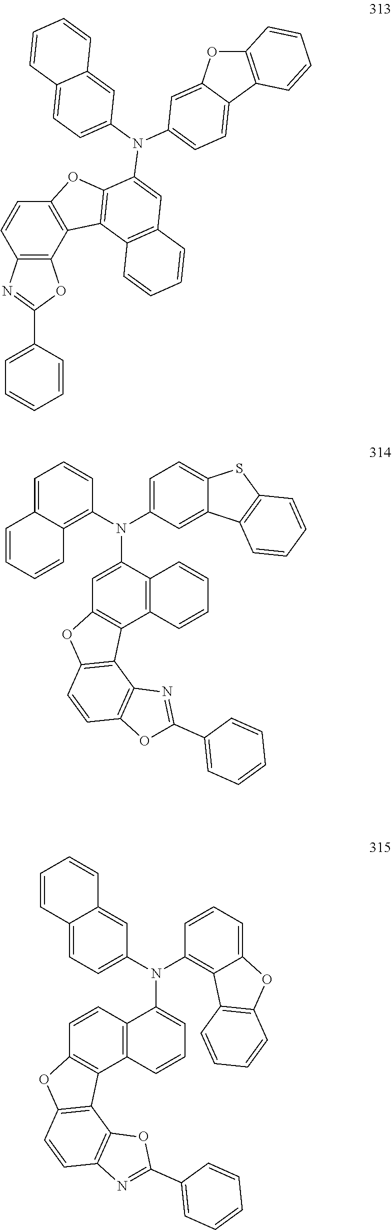 [SMILES: c1ccc(-c2nc3ccc4oc5c(N(c6ccc7ccccc7c6)c6ccc7c(c6)oc6ccccc67)cc6ccccc6c5c4c3o2)cc1 ; c1ccc(-c2nc3c(ccc4oc5cc(N(c6ccc7sc8ccccc8c7c6)c6cccc7ccccc67)c6ccccc6c5c43)o2)cc1 ; c1ccc(-c2nc3ccc4oc5ccc6c(N(c7ccc8ccccc8c7)c7cccc8oc9ccccc9c78)cccc6c5c4c3o2)cc1]

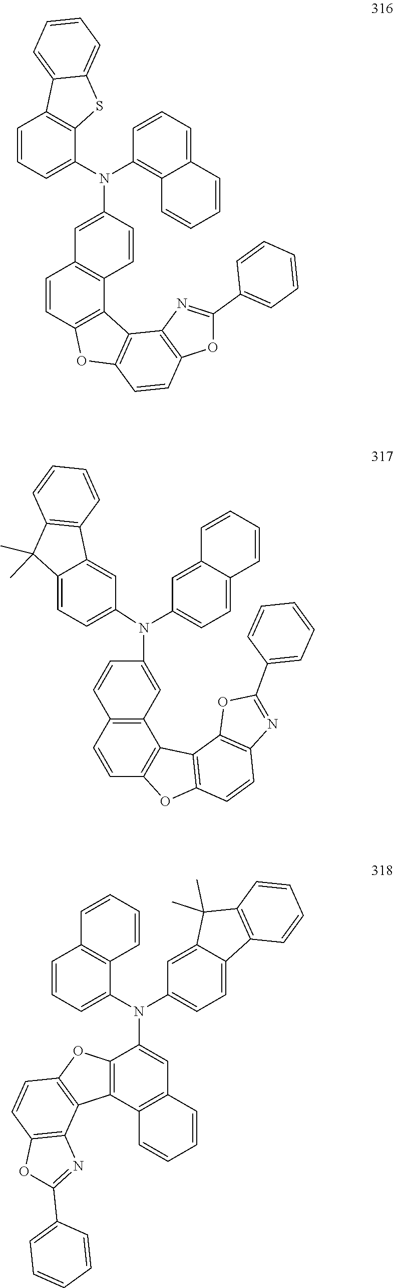 [SMILES: c1ccc(-c2nc3c(ccc4oc5ccc6cc(N(c7cccc8ccccc78)c7cccc8c7sc7ccccc78)ccc6c5c43)o2)cc1 ; CC1(C)c2ccccc2-c2cc(N(c3ccc4ccccc4c3)c3ccc4ccc5oc6ccc7nc(-c8ccccc8)oc7c6c5c4c3)ccc21 ; CC1(C)c2ccccc2-c2ccc(N(c3cccc4ccccc34)c3cc4ccccc4c4c3oc3ccc5oc(-c6ccccc6)nc5c34)cc21]

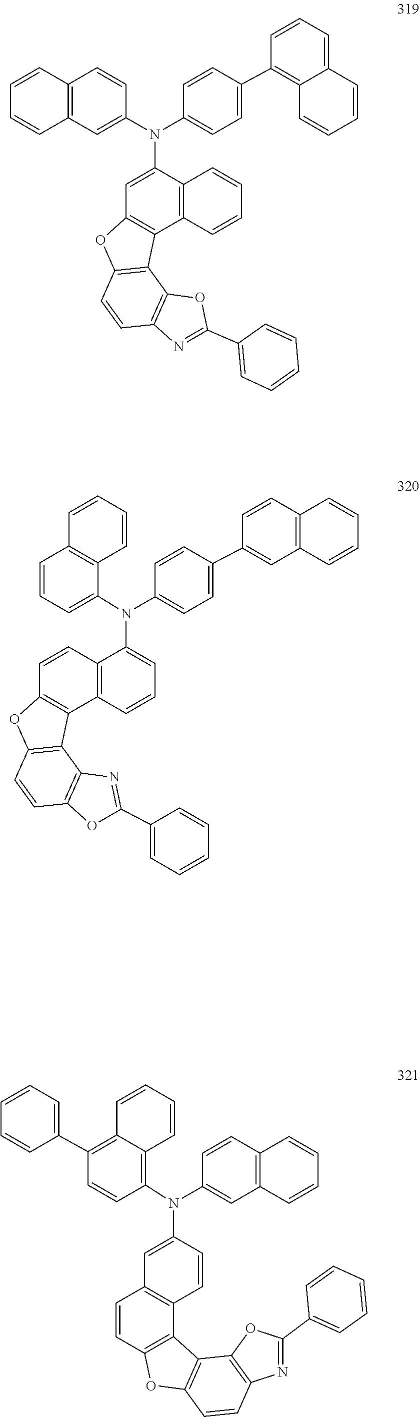 [SMILES: c1ccc(-c2nc3ccc4oc5cc(N(c6ccc(-c7cccc8ccccc78)cc6)c6ccc7ccccc7c6)c6ccccc6c5c4c3o2)cc1 ; c1ccc(-c2nc3c(ccc4oc5ccc6c(N(c7ccc(-c8ccc9ccccc9c8)cc7)c7cccc8ccccc78)cccc6c5c43)o2)cc1 ; c1ccc(-c2nc3ccc4oc5ccc6cc(N(c7ccc8ccccc8c7)c7ccc(-c8ccccc8)c8ccccc78)ccc6c5c4c3o2)cc1]

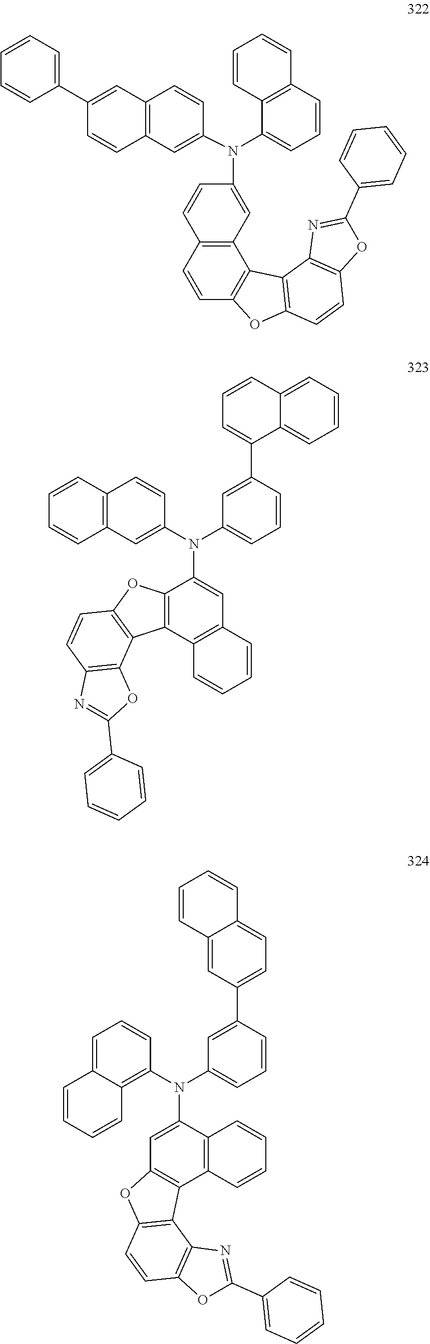 [SMILES: c1ccc(-c2ccc3cc(N(c4ccc5ccc6oc7ccc8oc(-c9ccccc9)nc8c7c6c5c4)c4cccc5ccccc45)ccc3c2)cc1 ; c1ccc(-c2nc3ccc4oc5c(N(c6cccc(-c7cccc8ccccc78)c6)c6ccc7ccccc7c6)cc6ccccc6c5c4c3o2)cc1 ; c1ccc(-c2nc3c(ccc4oc5cc(N(c6cccc(-c7ccc8ccccc8c7)c6)c6cccc7ccccc67)c6ccccc6c5c43)o2)cc1]

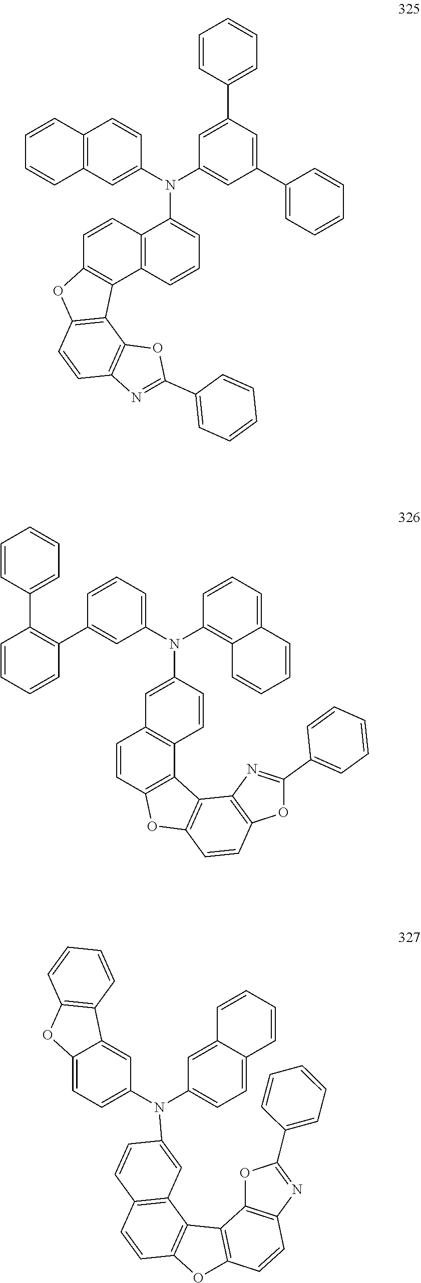 [SMILES: c1ccc(-c2cc(-c3ccccc3)cc(N(c3ccc4ccccc4c3)c3cccc4c3ccc3oc5ccc6nc(-c7ccccc7)oc6c5c34)c2)cc1 ; c1ccc(-c2nc3c(ccc4oc5ccc6cc(N(c7cccc(-c8ccccc8-c8ccccc8)c7)c7cccc8ccccc78)ccc6c5c43)o2)cc1 ; c1ccc(-c2nc3ccc4oc5ccc6ccc(N(c7ccc8ccccc8c7)c7ccc8oc9ccccc9c8c7)cc6c5c4c3o2)cc1]

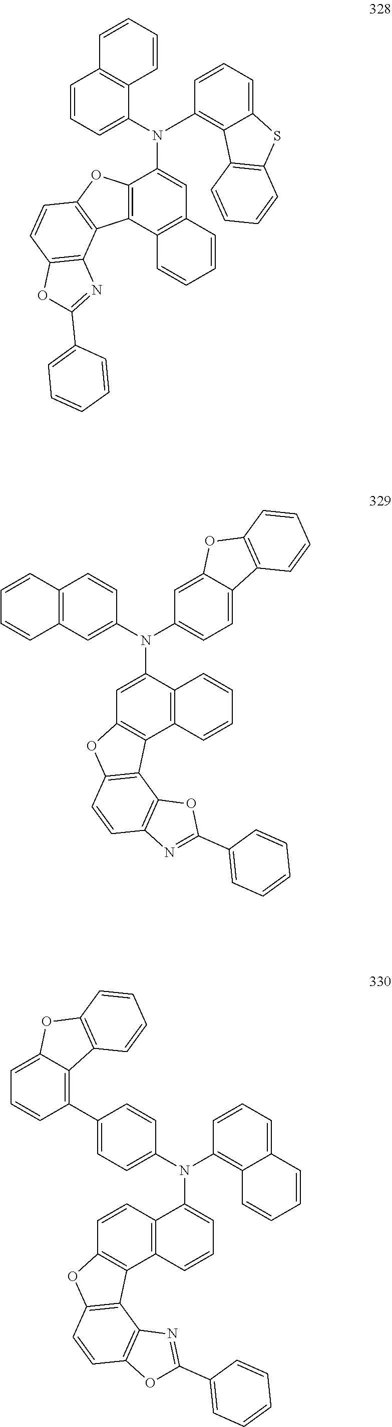 [SMILES: c1ccc(-c2nc3c(ccc4oc5c(N(c6cccc7ccccc67)c6cccc7sc8ccccc8c67)cc6ccccc6c5c43)o2)cc1 ; c1ccc(-c2nc3ccc4oc5cc(N(c6ccc7ccccc7c6)c6ccc7c(c6)oc6ccccc67)c6ccccc6c5c4c3o2)cc1 ; c1ccc(-c2nc3c(ccc4oc5ccc6c(N(c7ccc(-c8cccc9oc%10ccccc%10c89)cc7)c7cccc8ccccc78)cccc6c5c43)o2)cc1]

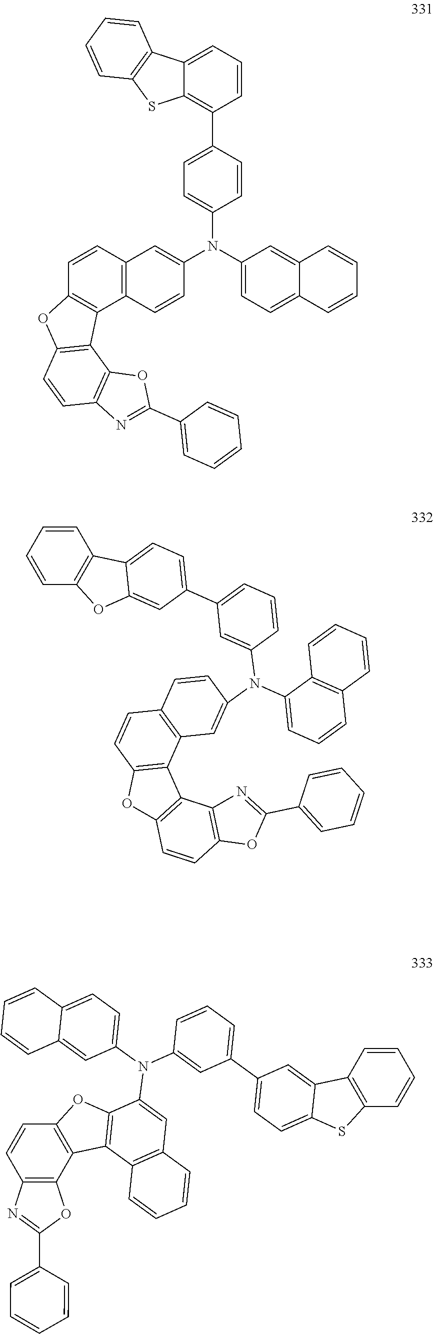 [SMILES: c1ccc(-c2nc3ccc4oc5ccc6cc(N(c7ccc(-c8cccc9c8sc8ccccc89)cc7)c7ccc8ccccc8c7)ccc6c5c4c3o2)cc1 ; c1ccc(-c2nc3c(ccc4oc5ccc6ccc(N(c7cccc(-c8ccc9c(c8)oc8ccccc89)c7)c7cccc8ccccc78)cc6c5c43)o2)cc1 ; c1ccc(-c2nc3ccc4oc5c(N(c6cccc(-c7ccc8sc9ccccc9c8c7)c6)c6ccc7ccccc7c6)cc6ccccc6c5c4c3o2)cc1]

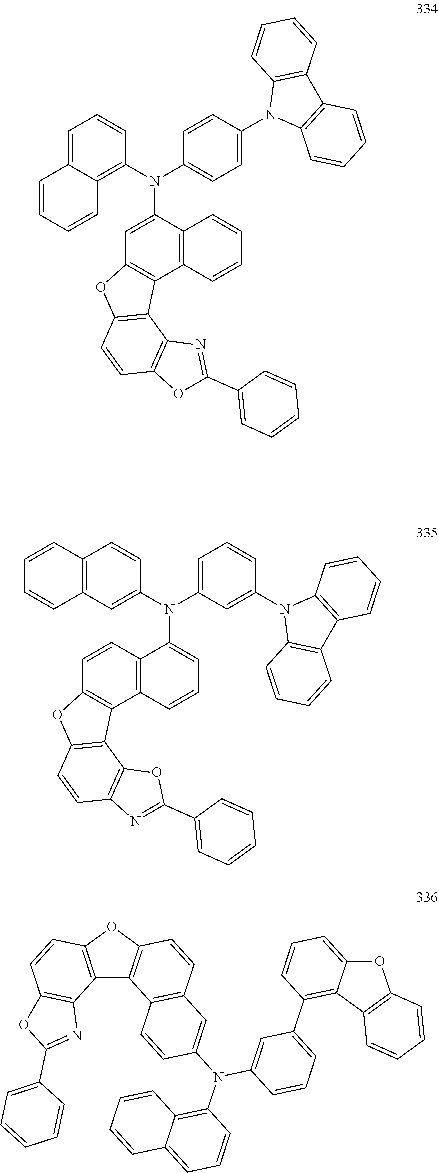 [SMILES: c1ccc(-c2nc3c(ccc4oc5cc(N(c6ccc(-n7c8ccccc8c8ccccc87)cc6)c6cccc7ccccc67)c6ccccc6c5c43)o2)cc1 ; c1ccc(-c2nc3ccc4oc5ccc6c(N(c7cccc(-n8c9ccccc9c9ccccc98)c7)c7ccc8ccccc8c7)cccc6c5c4c3o2)cc1 ; c1ccc(-c2nc3c(ccc4oc5ccc6cc(N(c7cccc(-c8cccc9oc%10ccccc%10c89)c7)c7cccc8ccccc78)ccc6c5c43)o2)cc1]

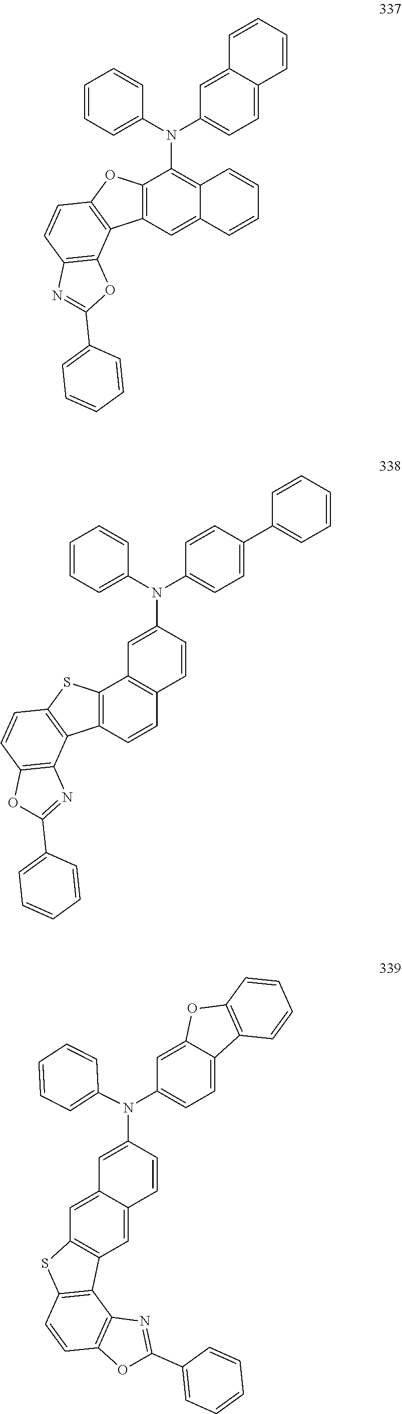 [SMILES: c1ccc(-c2nc3ccc4oc5c(N(c6ccccc6)c6ccc7ccccc7c6)c6ccccc6cc5c4c3o2)cc1 ; c1ccc(-c2ccc(N(c3ccccc3)c3ccc4ccc5c(sc6ccc7oc(-c8ccccc8)nc7c65)c4c3)cc2)cc1 ; c1ccc(-c2nc3c(ccc4sc5cc6cc(N(c7ccccc7)c7ccc8c(c7)oc7ccccc78)ccc6cc5c43)o2)cc1]

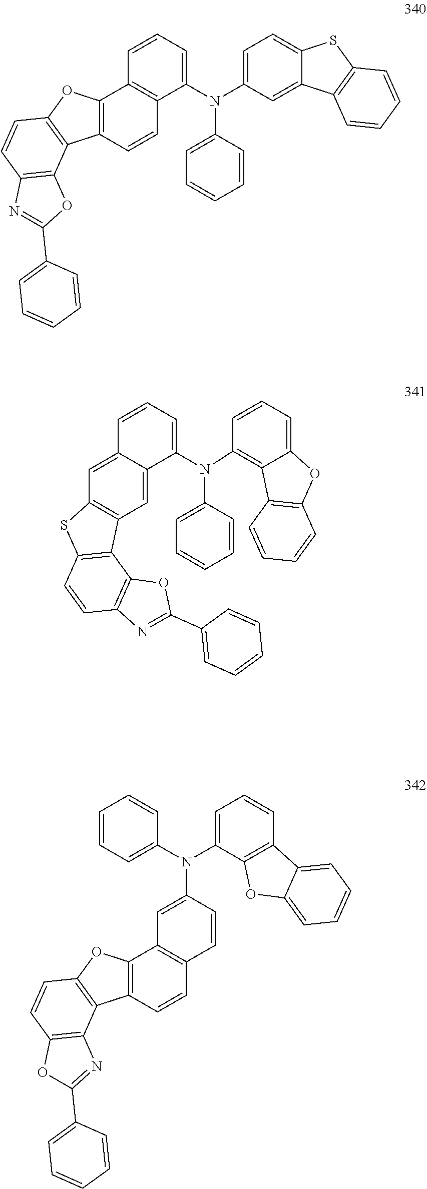 [SMILES: c1ccc(-c2nc3ccc4oc5c6cccc(N(c7ccccc7)c7ccc8sc9ccccc9c8c7)c6ccc5c4c3o2)cc1 ; c1ccc(-c2nc3ccc4sc5cc6cccc(N(c7ccccc7)c7cccc8oc9ccccc9c78)c6cc5c4c3o2)cc1 ; c1ccc(-c2nc3c(ccc4oc5c6cc(N(c7ccccc7)c7cccc8c7oc7ccccc78)ccc6ccc5c43)o2)cc1]

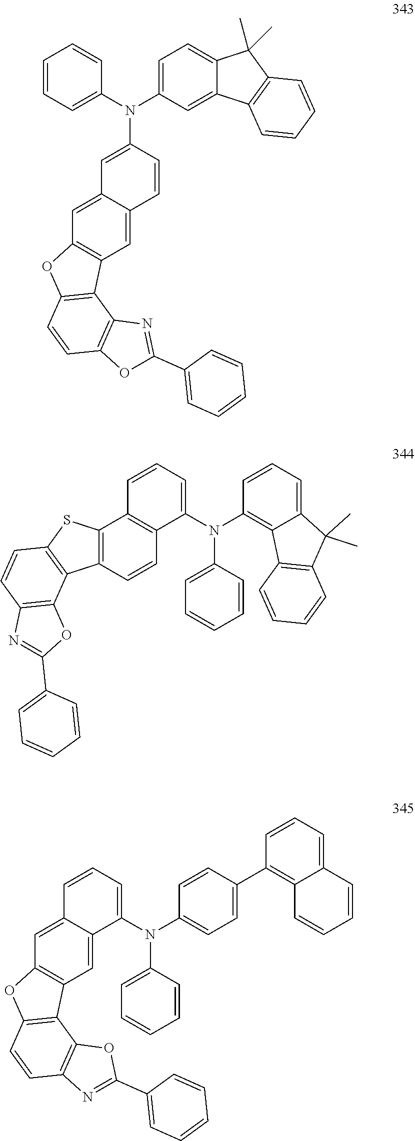 [SMILES: CC1(C)c2ccccc2-c2cc(N(c3ccccc3)c3ccc4cc5c(cc4c3)oc3ccc4oc(-c6ccccc6)nc4c35)ccc21 ; CC1(C)c2ccccc2-c2c(N(c3ccccc3)c3cccc4c3ccc3c4sc4ccc5nc(-c6ccccc6)oc5c43)cccc21 ; c1ccc(-c2nc3ccc4oc5cc6cccc(N(c7ccccc7)c7ccc(-c8cccc9ccccc89)cc7)c6cc5c4c3o2)cc1]

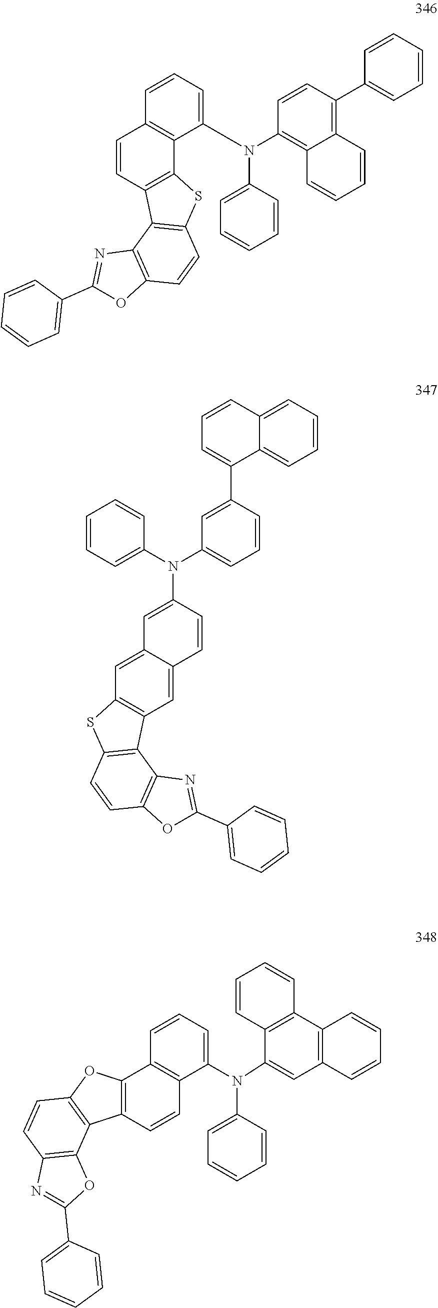 [SMILES: c1ccc(-c2nc3c(ccc4sc5c(ccc6cccc(N(c7ccccc7)c7ccc(-c8ccccc8)c8ccccc78)c65)c43)o2)cc1 ; c1ccc(-c2nc3c(ccc4sc5cc6cc(N(c7ccccc7)c7cccc(-c8cccc9ccccc89)c7)ccc6cc5c43)o2)cc1 ; c1ccc(-c2nc3ccc4oc5c6cccc(N(c7ccccc7)c7cc8ccccc8c8ccccc78)c6ccc5c4c3o2)cc1]

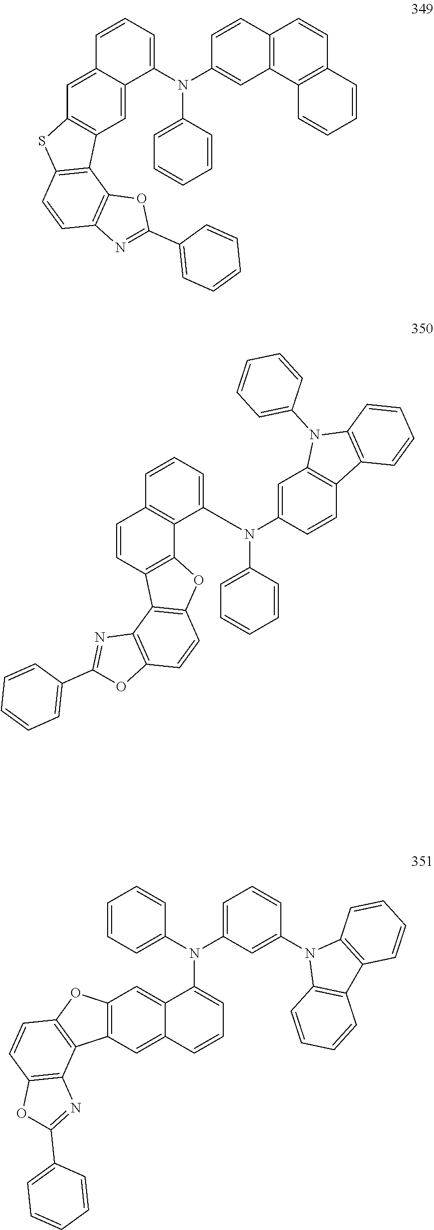 [SMILES: c1ccc(-c2nc3ccc4sc5cc6cccc(N(c7ccccc7)c7ccc8ccc9ccccc9c8c7)c6cc5c4c3o2)cc1 ; c1ccc(-c2nc3c(ccc4oc5c(ccc6cccc(N(c7ccccc7)c7ccc8c9ccccc9n(-c9ccccc9)c8c7)c65)c43)o2)cc1 ; c1ccc(-c2nc3c(ccc4oc5cc6c(N(c7ccccc7)c7cccc(-n8c9ccccc9c9ccccc98)c7)cccc6cc5c43)o2)cc1]

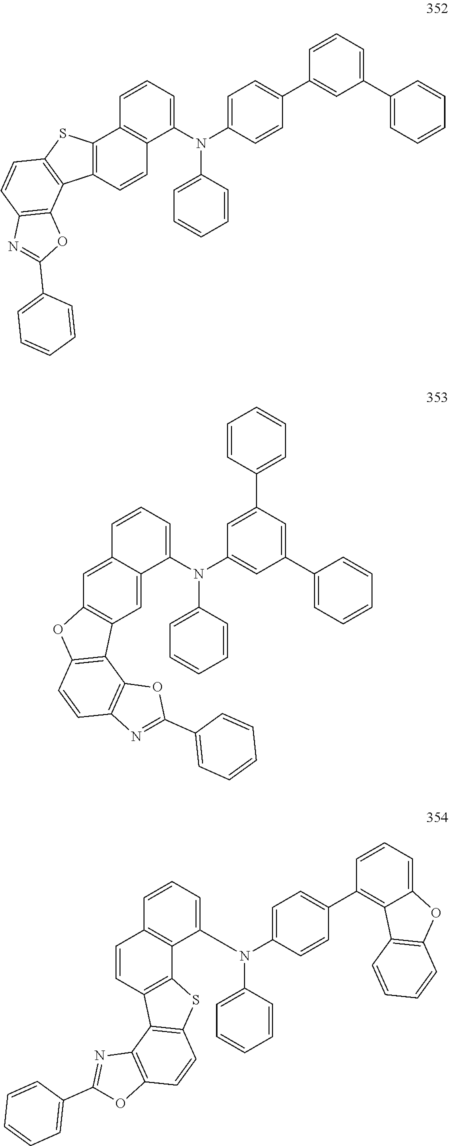 [SMILES: c1ccc(-c2cccc(-c3ccc(N(c4ccccc4)c4cccc5c4ccc4c5sc5ccc6nc(-c7ccccc7)oc6c54)cc3)c2)cc1 ; c1ccc(-c2cc(-c3ccccc3)cc(N(c3ccccc3)c3cccc4cc5oc6ccc7nc(-c8ccccc8)oc7c6c5cc34)c2)cc1 ; c1ccc(-c2nc3c(ccc4sc5c(ccc6cccc(N(c7ccccc7)c7ccc(-c8cccc9oc%10ccccc%10c89)cc7)c65)c43)o2)cc1]

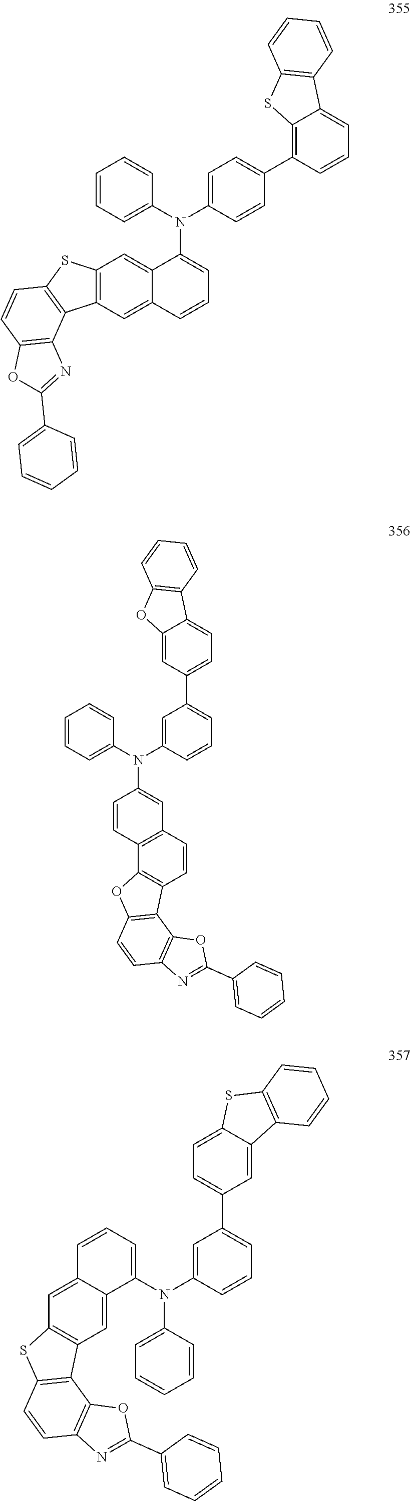 [SMILES: c1ccc(-c2nc3c(ccc4sc5cc6c(N(c7ccccc7)c7ccc(-c8cccc9c8sc8ccccc89)cc7)cccc6cc5c43)o2)cc1 ; c1ccc(-c2nc3ccc4oc5c6ccc(N(c7ccccc7)c7cccc(-c8ccc9c(c8)oc8ccccc89)c7)cc6ccc5c4c3o2)cc1 ; c1ccc(-c2nc3ccc4sc5cc6cccc(N(c7ccccc7)c7cccc(-c8ccc9sc%10ccccc%10c9c8)c7)c6cc5c4c3o2)cc1]

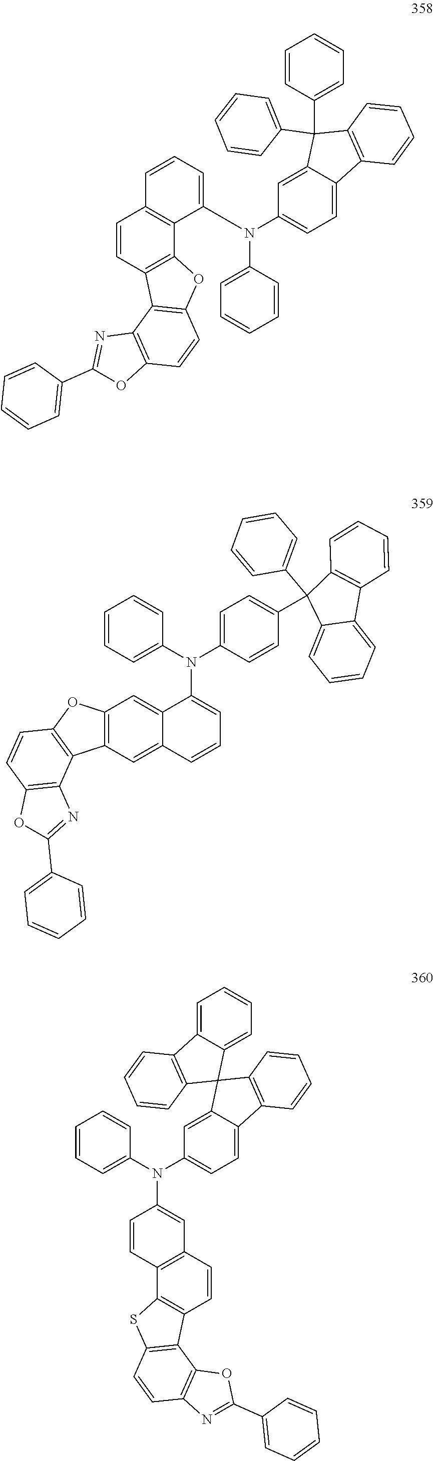 [SMILES: c1ccc(-c2nc3c(ccc4oc5c(ccc6cccc(N(c7ccccc7)c7ccc8c(c7)C(c7ccccc7)(c7ccccc7)c7ccccc7-8)c65)c43)o2)cc1 ; c1ccc(-c2nc3c(ccc4oc5cc6c(N(c7ccccc7)c7ccc(C8(c9ccccc9)c9ccccc9-c9ccccc98)cc7)cccc6cc5c43)o2)cc1 ; c1ccc(-c2nc3ccc4sc5c6ccc(N(c7ccccc7)c7ccc8c(c7)C7(c9ccccc9-c9ccccc97)c7ccccc7-8)cc6ccc5c4c3o2)cc1]

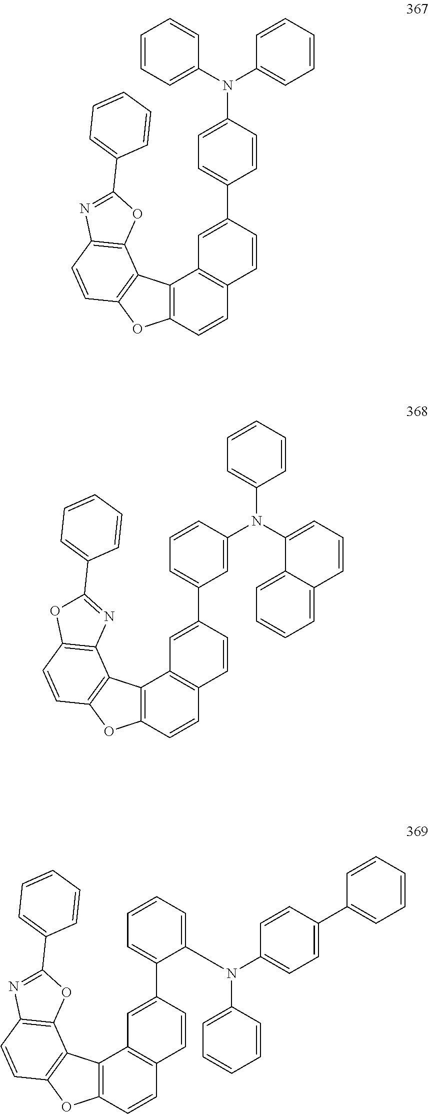 [SMILES: c1ccc(-c2nc3ccc4oc5ccc6ccc(-c7ccc(N(c8ccccc8)c8ccccc8)cc7)cc6c5c4c3o2)cc1 ; c1ccc(-c2nc3c(ccc4oc5ccc6ccc(-c7cccc(N(c8ccccc8)c8cccc9ccccc89)c7)cc6c5c43)o2)cc1 ; c1ccc(-c2ccc(N(c3ccccc3)c3ccccc3-c3ccc4ccc5oc6ccc7nc(-c8ccccc8)oc7c6c5c4c3)cc2)cc1]

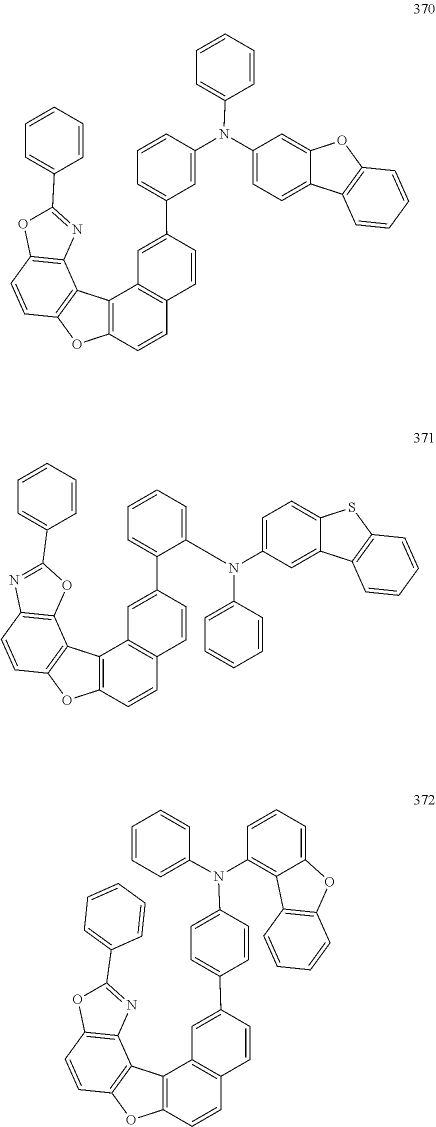 [SMILES: c1ccc(-c2nc3c(ccc4oc5ccc6ccc(-c7cccc(N(c8ccccc8)c8ccc9c(c8)oc8ccccc89)c7)cc6c5c43)o2)cc1 ; c1ccc(-c2nc3ccc4oc5ccc6ccc(-c7ccccc7N(c7ccccc7)c7ccc8sc9ccccc9c8c7)cc6c5c4c3o2)cc1 ; c1ccc(-c2nc3c(ccc4oc5ccc6ccc(-c7ccc(N(c8ccccc8)c8cccc9oc%10ccccc%10c89)cc7)cc6c5c43)o2)cc1]

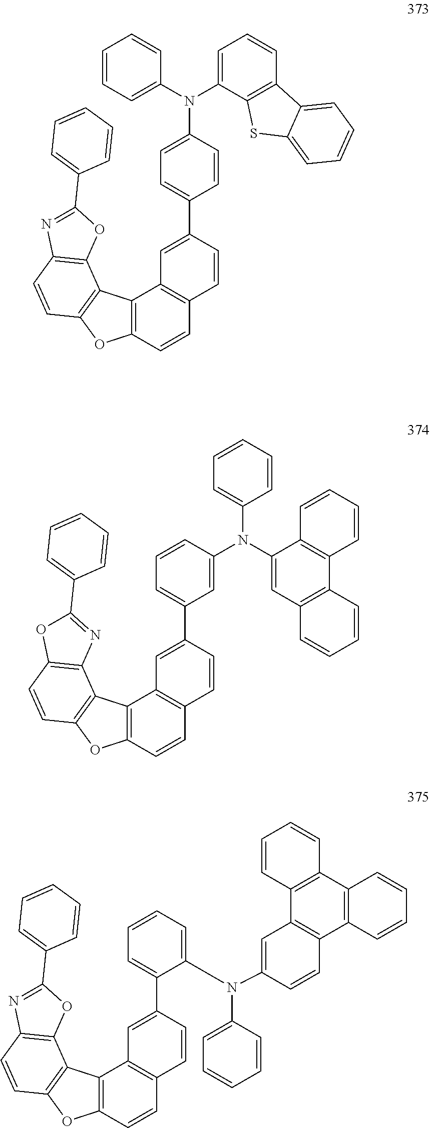 [SMILES: c1ccc(-c2nc3ccc4oc5ccc6ccc(-c7ccc(N(c8ccccc8)c8cccc9c8sc8ccccc89)cc7)cc6c5c4c3o2)cc1 ; c1ccc(-c2nc3c(ccc4oc5ccc6ccc(-c7cccc(N(c8ccccc8)c8cc9ccccc9c9ccccc89)c7)cc6c5c43)o2)cc1 ; c1ccc(-c2nc3ccc4oc5ccc6ccc(-c7ccccc7N(c7ccccc7)c7ccc8c9ccccc9c9ccccc9c8c7)cc6c5c4c3o2)cc1]

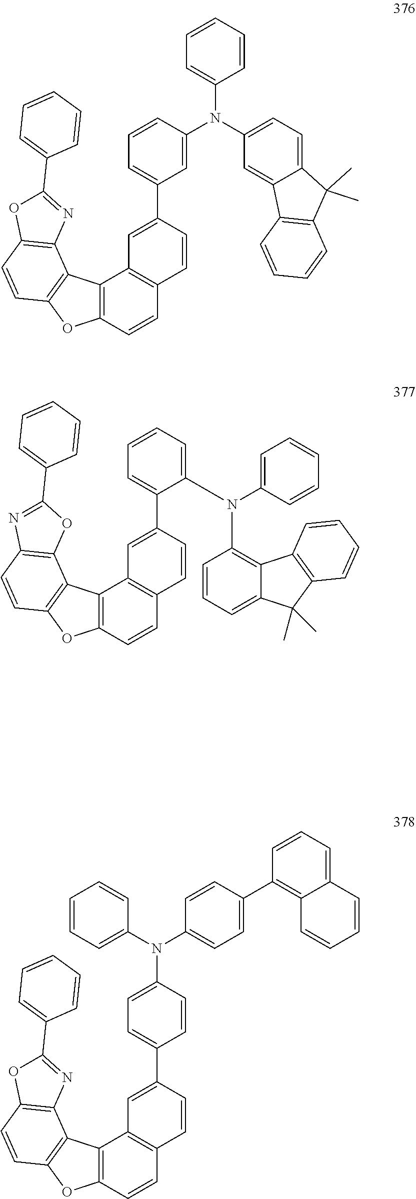 [SMILES: CC1(C)c2ccccc2-c2cc(N(c3ccccc3)c3cccc(-c4ccc5ccc6oc7ccc8oc(-c9ccccc9)nc8c7c6c5c4)c3)ccc21 ; CC1(C)c2ccccc2-c2c(N(c3ccccc3)c3ccccc3-c3ccc4ccc5oc6ccc7nc(-c8ccccc8)oc7c6c5c4c3)cccc21 ; c1ccc(-c2nc3c(ccc4oc5ccc6ccc(-c7ccc(N(c8ccccc8)c8ccc(-c9cccc%10ccccc9%10)cc8)cc7)cc6c5c43)o2)cc1]

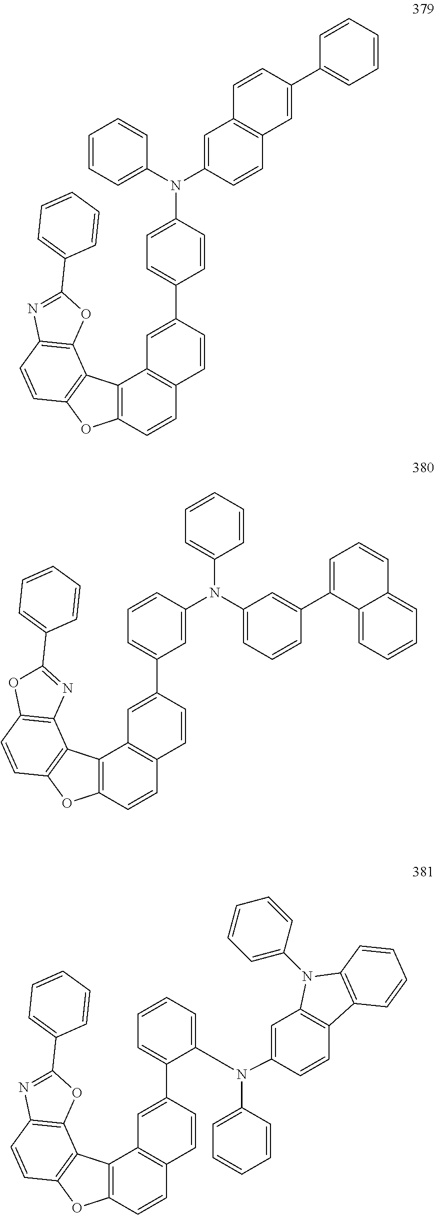 [SMILES: c1ccc(-c2ccc3cc(N(c4ccccc4)c4ccc(-c5ccc6ccc7oc8ccc9nc(-c%10ccccc%10)oc9c8c7c6c5)cc4)ccc3c2)cc1 ; c1ccc(-c2nc3c(ccc4oc5ccc6ccc(-c7cccc(N(c8ccccc8)c8cccc(-c9cccc%10ccccc9%10)c8)c7)cc6c5c43)o2)cc1 ; c1ccc(-c2nc3ccc4oc5ccc6ccc(-c7ccccc7N(c7ccccc7)c7ccc8c9ccccc9n(-c9ccccc9)c8c7)cc6c5c4c3o2)cc1]

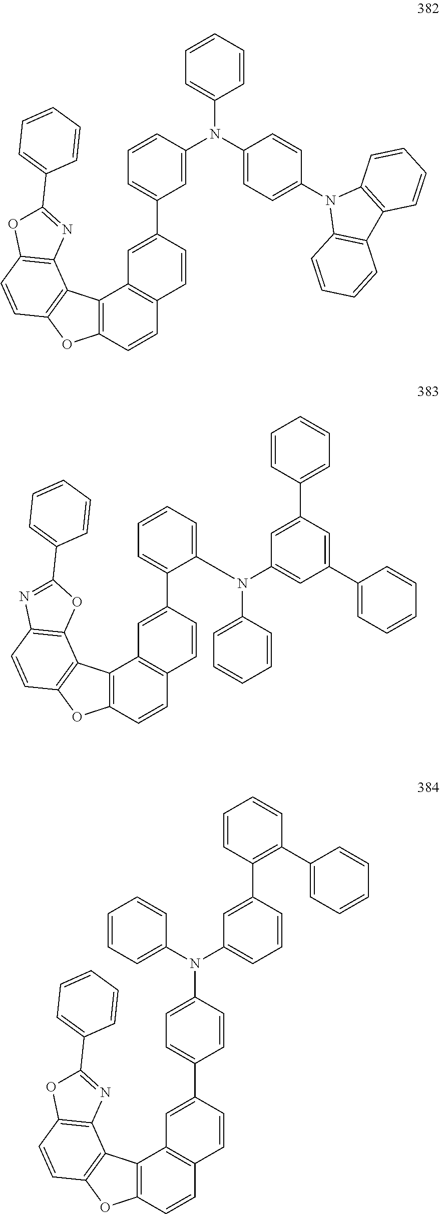 [SMILES: c1ccc(-c2nc3c(ccc4oc5ccc6ccc(-c7cccc(N(c8ccccc8)c8ccc(-n9c%10ccccc%10c%10ccccc%109)cc8)c7)cc6c5c43)o2)cc1 ; c1ccc(-c2cc(-c3ccccc3)cc(N(c3ccccc3)c3ccccc3-c3ccc4ccc5oc6ccc7nc(-c8ccccc8)oc7c6c5c4c3)c2)cc1 ; c1ccc(-c2nc3c(ccc4oc5ccc6ccc(-c7ccc(N(c8ccccc8)c8cccc(-c9ccccc9-c9ccccc9)c8)cc7)cc6c5c43)o2)cc1]

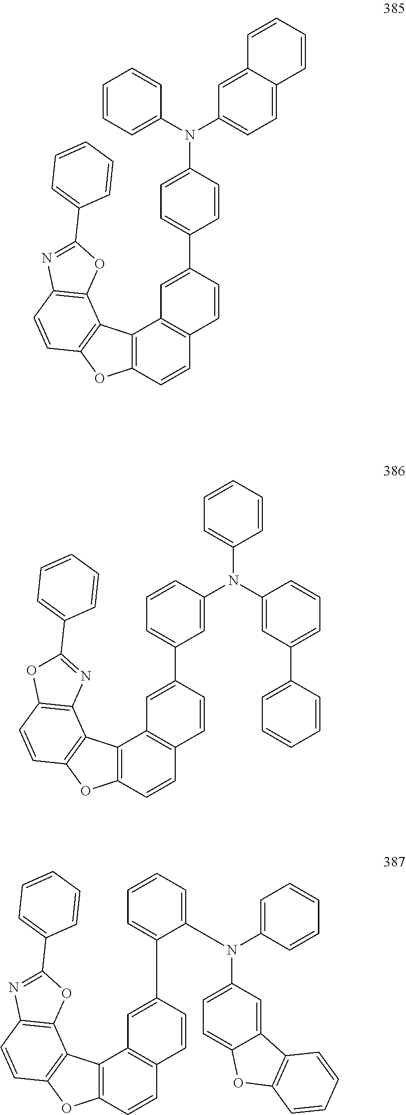 [SMILES: c1ccc(-c2nc3ccc4oc5ccc6ccc(-c7ccc(N(c8ccccc8)c8ccc9ccccc9c8)cc7)cc6c5c4c3o2)cc1 ; c1ccc(-c2cccc(N(c3ccccc3)c3cccc(-c4ccc5ccc6oc7ccc8oc(-c9ccccc9)nc8c7c6c5c4)c3)c2)cc1 ; c1ccc(-c2nc3ccc4oc5ccc6ccc(-c7ccccc7N(c7ccccc7)c7ccc8oc9ccccc9c8c7)cc6c5c4c3o2)cc1]

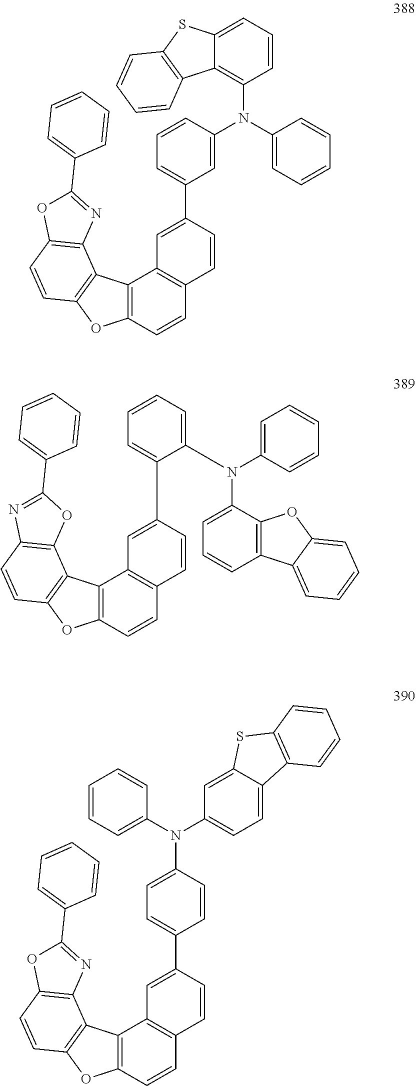 [SMILES: c1ccc(-c2nc3c(ccc4oc5ccc6ccc(-c7cccc(N(c8ccccc8)c8cccc9sc%10ccccc%10c89)c7)cc6c5c43)o2)cc1 ; c1ccc(-c2nc3ccc4oc5ccc6ccc(-c7ccccc7N(c7ccccc7)c7cccc8c7oc7ccccc78)cc6c5c4c3o2)cc1 ; c1ccc(-c2nc3c(ccc4oc5ccc6ccc(-c7ccc(N(c8ccccc8)c8ccc9c(c8)sc8ccccc89)cc7)cc6c5c43)o2)cc1]

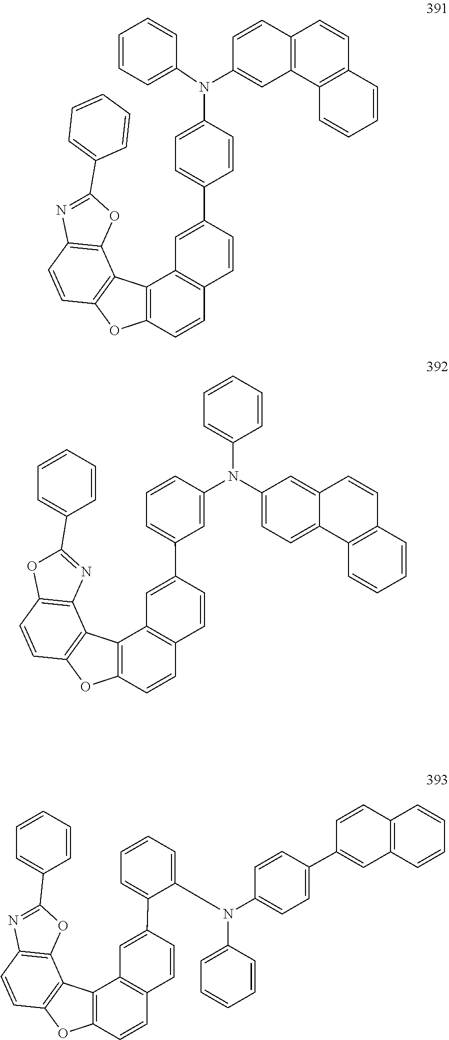 [SMILES: c1ccc(-c2nc3ccc4oc5ccc6ccc(-c7ccc(N(c8ccccc8)c8ccc9ccc%10ccccc%10c9c8)cc7)cc6c5c4c3o2)cc1 ; c1ccc(-c2nc3c(ccc4oc5ccc6ccc(-c7cccc(N(c8ccccc8)c8ccc9c(ccc%10ccccc%109)c8)c7)cc6c5c43)o2)cc1 ; c1ccc(-c2nc3ccc4oc5ccc6ccc(-c7ccccc7N(c7ccccc7)c7ccc(-c8ccc9ccccc9c8)cc7)cc6c5c4c3o2)cc1]

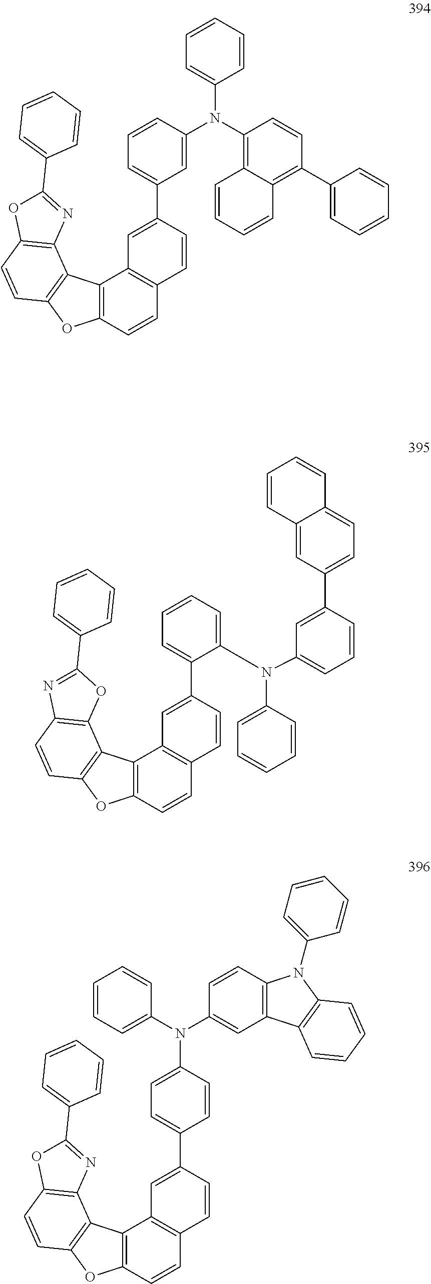 [SMILES: c1ccc(-c2nc3c(ccc4oc5ccc6ccc(-c7cccc(N(c8ccccc8)c8ccc(-c9ccccc9)c9ccccc89)c7)cc6c5c43)o2)cc1 ; c1ccc(-c2nc3ccc4oc5ccc6ccc(-c7ccccc7N(c7ccccc7)c7cccc(-c8ccc9ccccc9c8)c7)cc6c5c4c3o2)cc1 ; c1ccc(-c2nc3c(ccc4oc5ccc6ccc(-c7ccc(N(c8ccccc8)c8ccc9c(c8)c8ccccc8n9-c8ccccc8)cc7)cc6c5c43)o2)cc1]

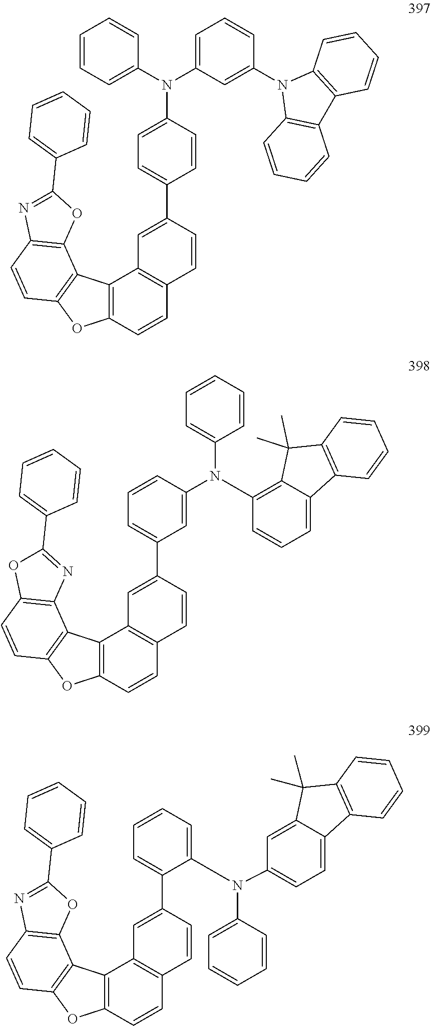 [SMILES: c1ccc(-c2nc3ccc4oc5ccc6ccc(-c7ccc(N(c8ccccc8)c8cccc(-n9c%10ccccc%10c%10ccccc%109)c8)cc7)cc6c5c4c3o2)cc1 ; CC1(C)c2ccccc2-c2cccc(N(c3ccccc3)c3cccc(-c4ccc5ccc6oc7ccc8oc(-c9ccccc9)nc8c7c6c5c4)c3)c21 ; CC1(C)c2ccccc2-c2ccc(N(c3ccccc3)c3ccccc3-c3ccc4ccc5oc6ccc7nc(-c8ccccc8)oc7c6c5c4c3)cc21]

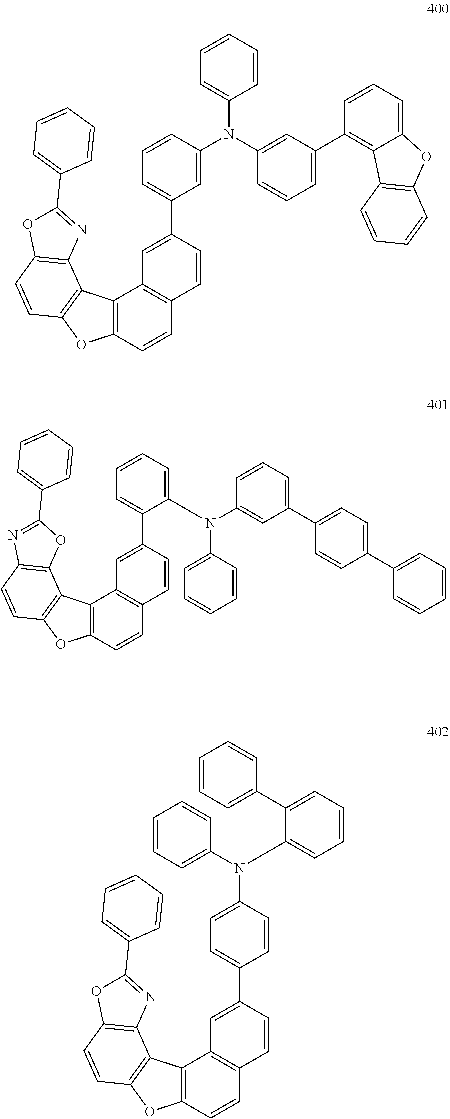 [SMILES: c1ccc(-c2nc3c(ccc4oc5ccc6ccc(-c7cccc(N(c8ccccc8)c8cccc(-c9cccc%10oc%11ccccc%11c9%10)c8)c7)cc6c5c43)o2)cc1 ; c1ccc(-c2ccc(-c3cccc(N(c4ccccc4)c4ccccc4-c4ccc5ccc6oc7ccc8nc(-c9ccccc9)oc8c7c6c5c4)c3)cc2)cc1 ; c1ccc(-c2nc3c(ccc4oc5ccc6ccc(-c7ccc(N(c8ccccc8)c8ccccc8-c8ccccc8)cc7)cc6c5c43)o2)cc1]

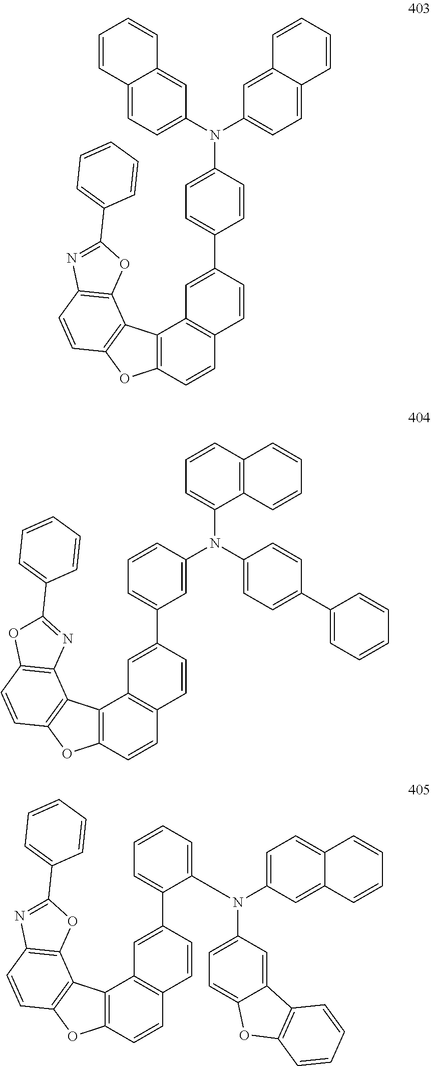 [SMILES: c1ccc(-c2nc3ccc4oc5ccc6ccc(-c7ccc(N(c8ccc9ccccc9c8)c8ccc9ccccc9c8)cc7)cc6c5c4c3o2)cc1 ; c1ccc(-c2ccc(N(c3cccc(-c4ccc5ccc6oc7ccc8oc(-c9ccccc9)nc8c7c6c5c4)c3)c3cccc4ccccc34)cc2)cc1 ; c1ccc(-c2nc3ccc4oc5ccc6ccc(-c7ccccc7N(c7ccc8ccccc8c7)c7ccc8oc9ccccc9c8c7)cc6c5c4c3o2)cc1]

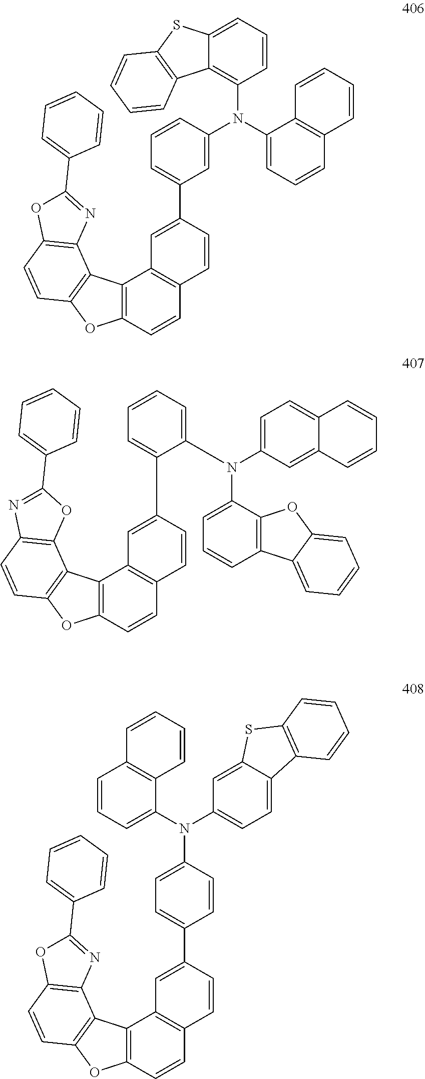 [SMILES: c1ccc(-c2nc3c(ccc4oc5ccc6ccc(-c7cccc(N(c8cccc9ccccc89)c8cccc9sc%10ccccc%10c89)c7)cc6c5c43)o2)cc1 ; c1ccc(-c2nc3ccc4oc5ccc6ccc(-c7ccccc7N(c7ccc8ccccc8c7)c7cccc8c7oc7ccccc78)cc6c5c4c3o2)cc1 ; c1ccc(-c2nc3c(ccc4oc5ccc6ccc(-c7ccc(N(c8ccc9c(c8)sc8ccccc89)c8cccc9ccccc89)cc7)cc6c5c43)o2)cc1]

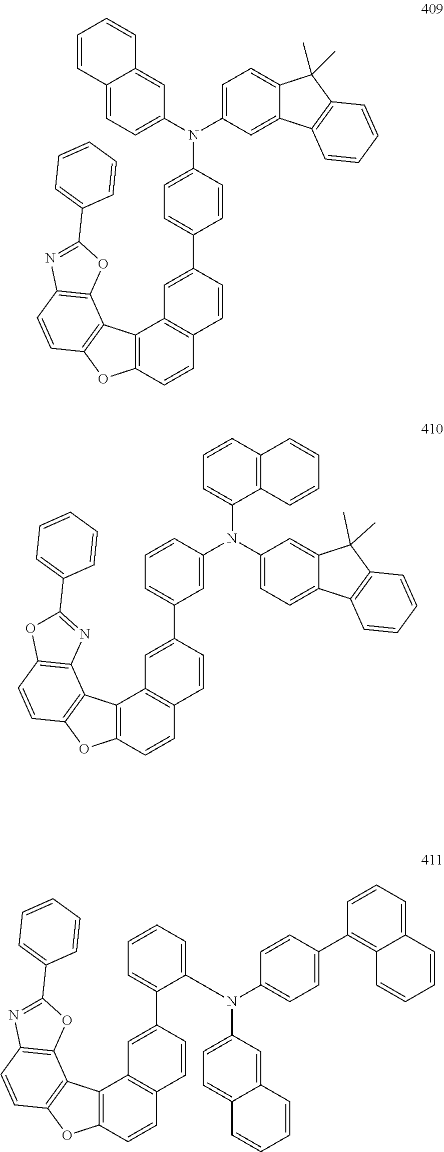 [SMILES: CC1(C)c2ccccc2-c2cc(N(c3ccc(-c4ccc5ccc6oc7ccc8nc(-c9ccccc9)oc8c7c6c5c4)cc3)c3ccc4ccccc4c3)ccc21 ; CC1(C)c2ccccc2-c2ccc(N(c3cccc(-c4ccc5ccc6oc7ccc8oc(-c9ccccc9)nc8c7c6c5c4)c3)c3cccc4ccccc34)cc21 ; c1ccc(-c2nc3ccc4oc5ccc6ccc(-c7ccccc7N(c7ccc(-c8cccc9ccccc89)cc7)c7ccc8ccccc8c7)cc6c5c4c3o2)cc1]

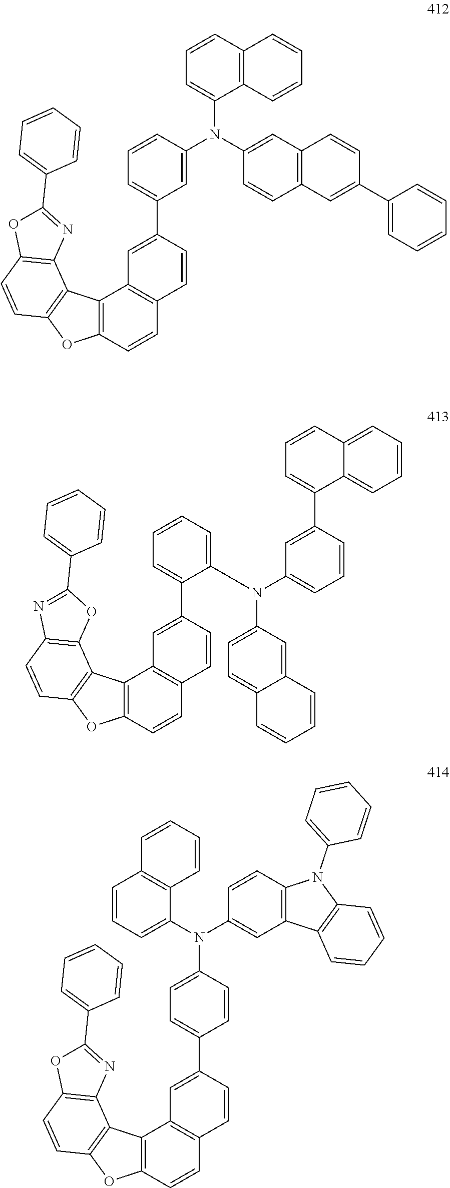 [SMILES: c1ccc(-c2ccc3cc(N(c4cccc(-c5ccc6ccc7oc8ccc9oc(-c%10ccccc%10)nc9c8c7c6c5)c4)c4cccc5ccccc45)ccc3c2)cc1 ; c1ccc(-c2nc3ccc4oc5ccc6ccc(-c7ccccc7N(c7cccc(-c8cccc9ccccc89)c7)c7ccc8ccccc8c7)cc6c5c4c3o2)cc1 ; c1ccc(-c2nc3c(ccc4oc5ccc6ccc(-c7ccc(N(c8ccc9c(c8)c8ccccc8n9-c8ccccc8)c8cccc9ccccc89)cc7)cc6c5c43)o2)cc1]

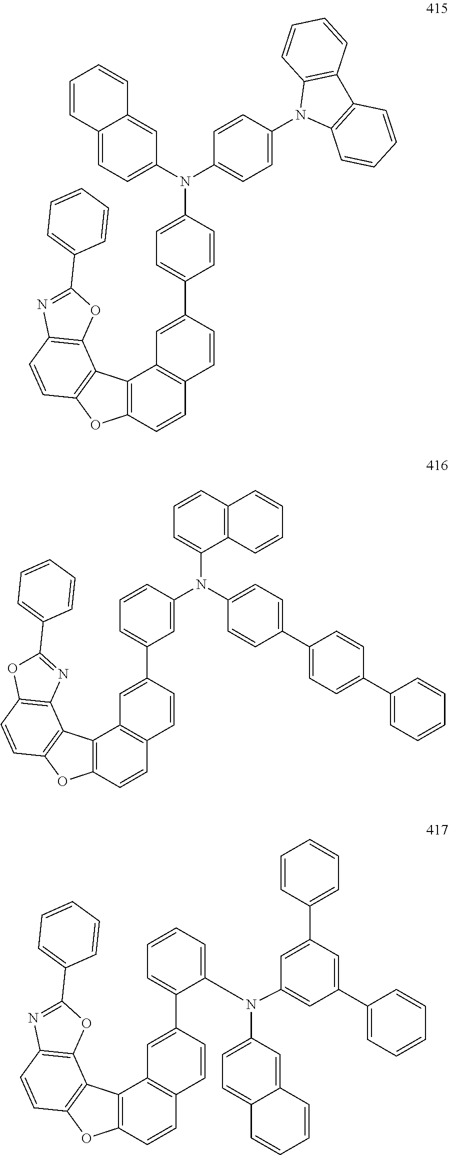 [SMILES: c1ccc(-c2nc3ccc4oc5ccc6ccc(-c7ccc(N(c8ccc(-n9c%10ccccc%10c%10ccccc%109)cc8)c8ccc9ccccc9c8)cc7)cc6c5c4c3o2)cc1 ; c1ccc(-c2ccc(-c3ccc(N(c4cccc(-c5ccc6ccc7oc8ccc9oc(-c%10ccccc%10)nc9c8c7c6c5)c4)c4cccc5ccccc45)cc3)cc2)cc1 ; c1ccc(-c2cc(-c3ccccc3)cc(N(c3ccc4ccccc4c3)c3ccccc3-c3ccc4ccc5oc6ccc7nc(-c8ccccc8)oc7c6c5c4c3)c2)cc1]

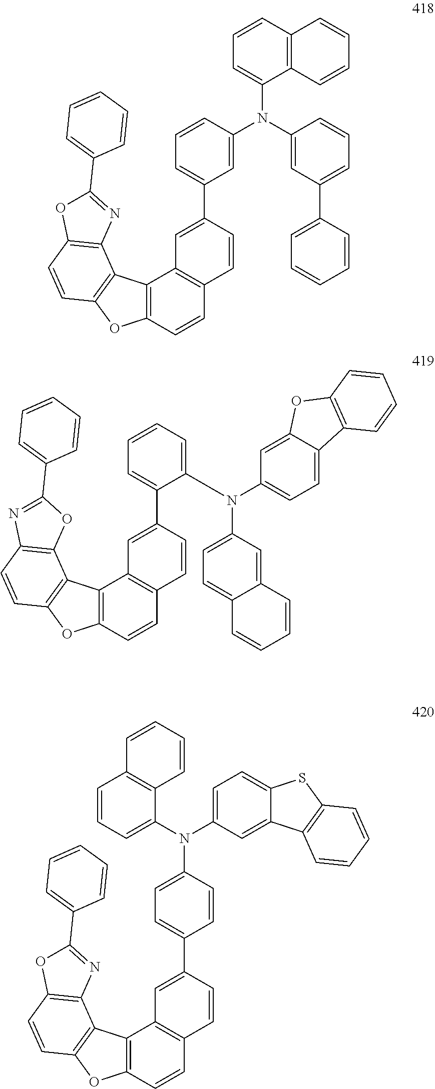 [SMILES: c1ccc(-c2cccc(N(c3cccc(-c4ccc5ccc6oc7ccc8oc(-c9ccccc9)nc8c7c6c5c4)c3)c3cccc4ccccc34)c2)cc1 ; c1ccc(-c2nc3ccc4oc5ccc6ccc(-c7ccccc7N(c7ccc8ccccc8c7)c7ccc8c(c7)oc7ccccc78)cc6c5c4c3o2)cc1 ; c1ccc(-c2nc3c(ccc4oc5ccc6ccc(-c7ccc(N(c8ccc9sc%10ccccc%10c9c8)c8cccc9ccccc89)cc7)cc6c5c43)o2)cc1]

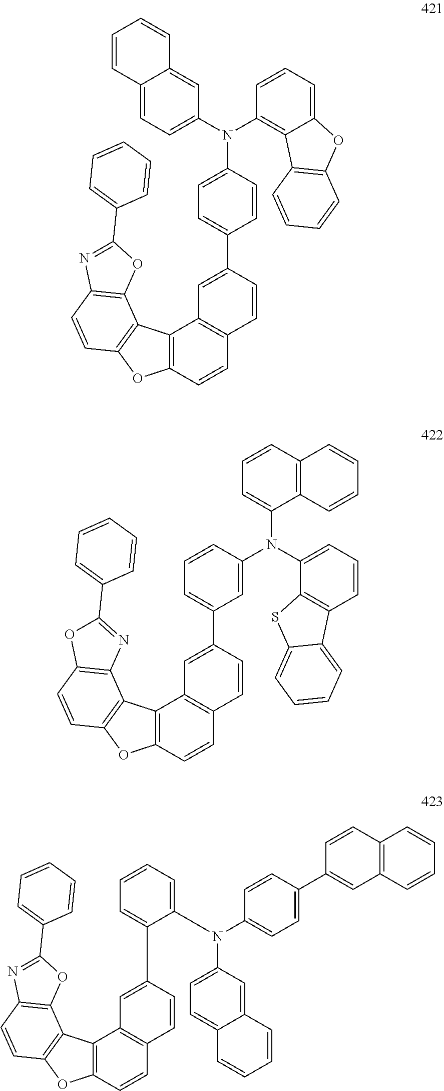 [SMILES: c1ccc(-c2nc3ccc4oc5ccc6ccc(-c7ccc(N(c8ccc9ccccc9c8)c8cccc9oc%10ccccc%10c89)cc7)cc6c5c4c3o2)cc1 ; c1ccc(-c2nc3c(ccc4oc5ccc6ccc(-c7cccc(N(c8cccc9ccccc89)c8cccc9c8sc8ccccc89)c7)cc6c5c43)o2)cc1 ; c1ccc(-c2nc3ccc4oc5ccc6ccc(-c7ccccc7N(c7ccc(-c8ccc9ccccc9c8)cc7)c7ccc8ccccc8c7)cc6c5c4c3o2)cc1]

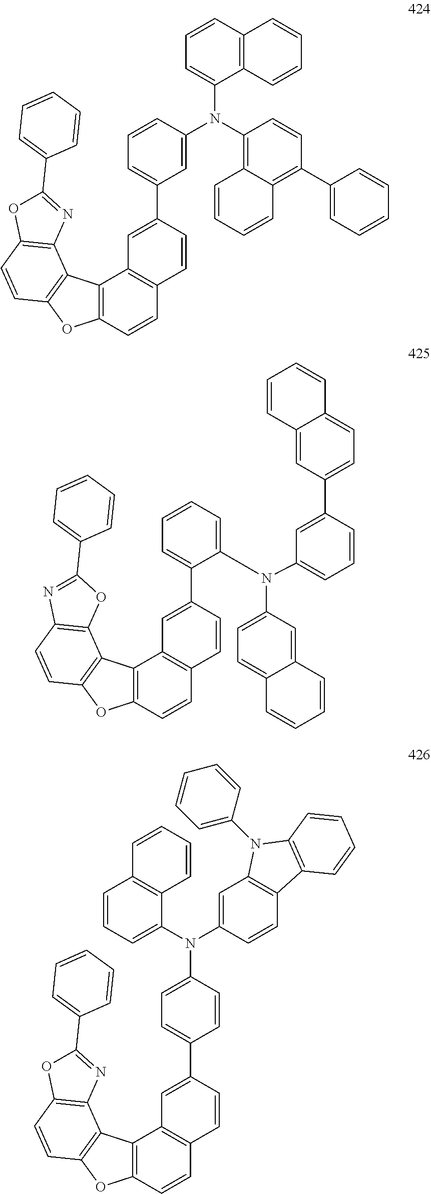 [SMILES: c1ccc(-c2nc3c(ccc4oc5ccc6ccc(-c7cccc(N(c8cccc9ccccc89)c8ccc(-c9ccccc9)c9ccccc89)c7)cc6c5c43)o2)cc1 ; c1ccc(-c2nc3ccc4oc5ccc6ccc(-c7ccccc7N(c7cccc(-c8ccc9ccccc9c8)c7)c7ccc8ccccc8c7)cc6c5c4c3o2)cc1 ; c1ccc(-c2nc3c(ccc4oc5ccc6ccc(-c7ccc(N(c8ccc9c%10ccccc%10n(-c%10ccccc%10)c9c8)c8cccc9ccccc89)cc7)cc6c5c43)o2)cc1]

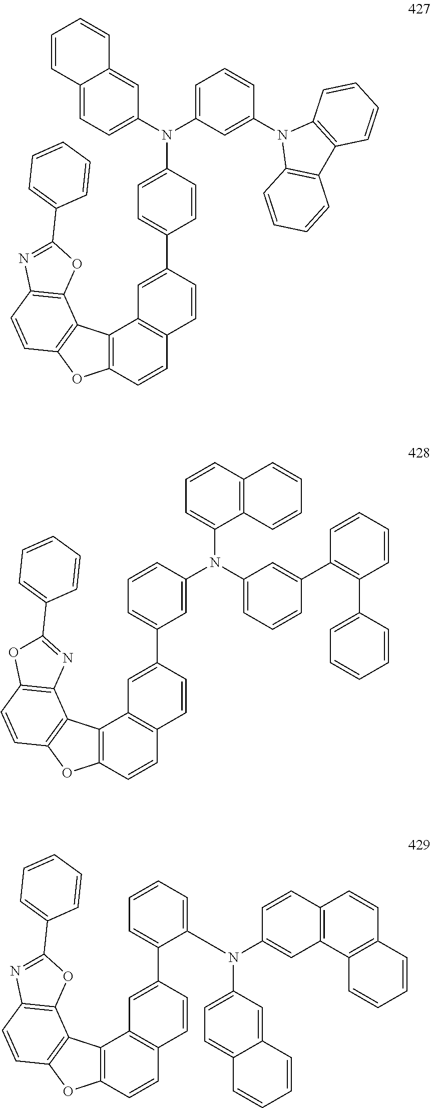 [SMILES: c1ccc(-c2nc3ccc4oc5ccc6ccc(-c7ccc(N(c8cccc(-n9c%10ccccc%10c%10ccccc%109)c8)c8ccc9ccccc9c8)cc7)cc6c5c4c3o2)cc1 ; c1ccc(-c2nc3c(ccc4oc5ccc6ccc(-c7cccc(N(c8cccc(-c9ccccc9-c9ccccc9)c8)c8cccc9ccccc89)c7)cc6c5c43)o2)cc1 ; c1ccc(-c2nc3ccc4oc5ccc6ccc(-c7ccccc7N(c7ccc8ccccc8c7)c7ccc8ccc9ccccc9c8c7)cc6c5c4c3o2)cc1]

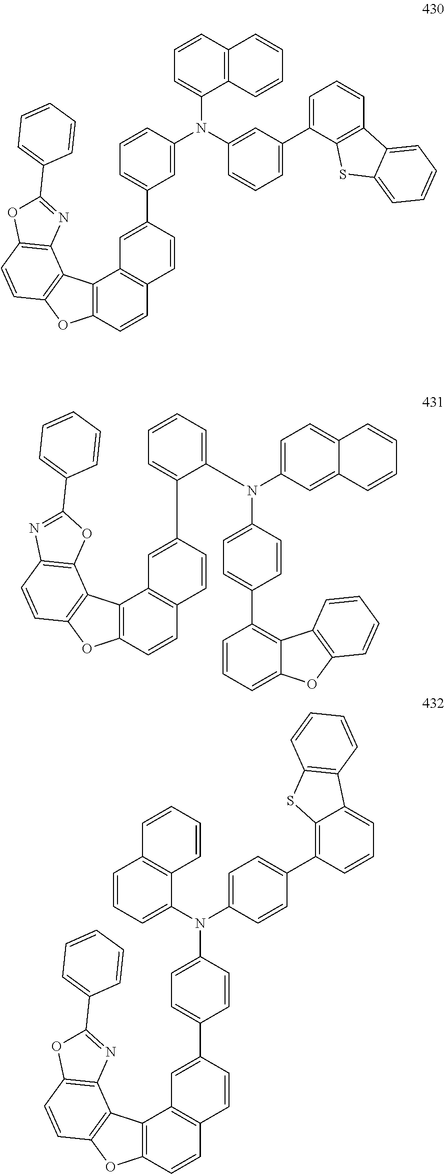

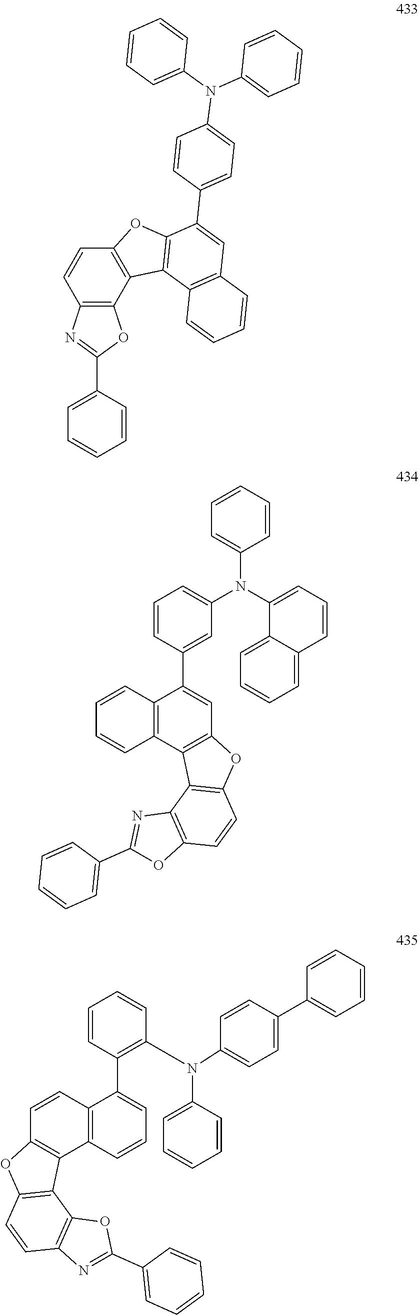 [SMILES: c1ccc(-c2nc3ccc4oc5c(-c6ccc(N(c7ccccc7)c7ccccc7)cc6)cc6ccccc6c5c4c3o2)cc1 ; c1ccc(-c2nc3c(ccc4oc5cc(-c6cccc(N(c7ccccc7)c7cccc8ccccc78)c6)c6ccccc6c5c43)o2)cc1 ; c1ccc(-c2ccc(N(c3ccccc3)c3ccccc3-c3cccc4c3ccc3oc5ccc6nc(-c7ccccc7)oc6c5c34)cc2)cc1]

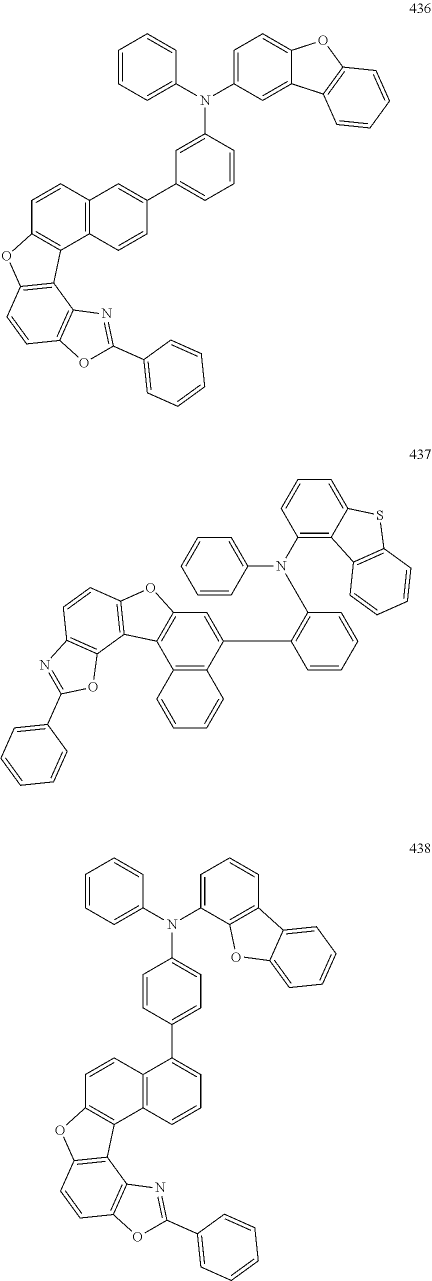 [SMILES: c1ccc(-c2nc3c(ccc4oc5ccc6cc(-c7cccc(N(c8ccccc8)c8ccc9oc%10ccccc%10c9c8)c7)ccc6c5c43)o2)cc1 ; c1ccc(-c2nc3ccc4oc5cc(-c6ccccc6N(c6ccccc6)c6cccc7sc8ccccc8c67)c6ccccc6c5c4c3o2)cc1 ; c1ccc(-c2nc3c(ccc4oc5ccc6c(-c7ccc(N(c8ccccc8)c8cccc9c8oc8ccccc89)cc7)cccc6c5c43)o2)cc1]

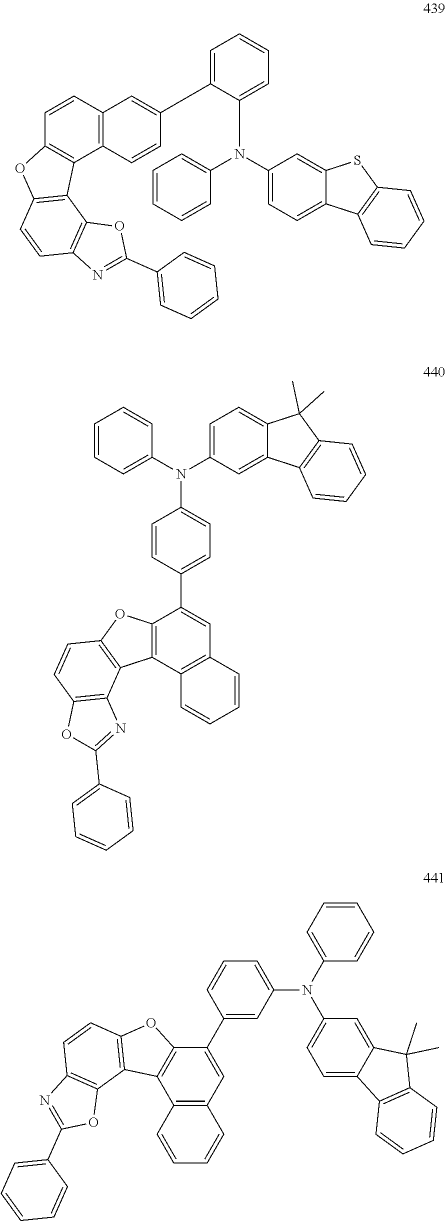 [SMILES: c1ccc(-c2nc3ccc4oc5ccc6cc(-c7ccccc7N(c7ccccc7)c7ccc8c(c7)sc7ccccc78)ccc6c5c4c3o2)cc1 ; CC1(C)c2ccccc2-c2cc(N(c3ccccc3)c3ccc(-c4cc5ccccc5c5c4oc4ccc6oc(-c7ccccc7)nc6c45)cc3)ccc21 ; CC1(C)c2ccccc2-c2ccc(N(c3ccccc3)c3cccc(-c4cc5ccccc5c5c4oc4ccc6nc(-c7ccccc7)oc6c45)c3)cc21]

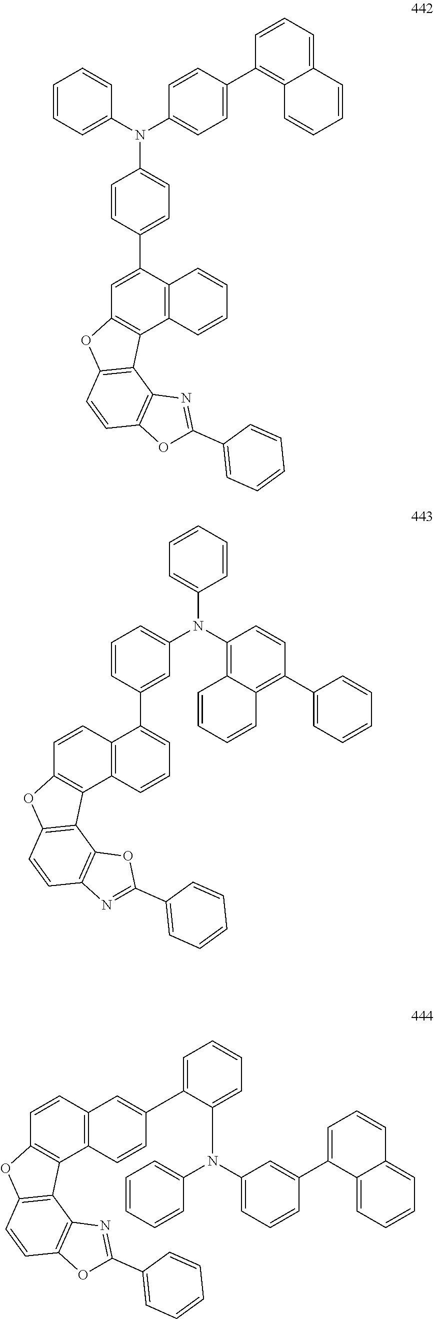 [SMILES: c1ccc(-c2nc3c(ccc4oc5cc(-c6ccc(N(c7ccccc7)c7ccc(-c8cccc9ccccc89)cc7)cc6)c6ccccc6c5c43)o2)cc1 ; c1ccc(-c2nc3ccc4oc5ccc6c(-c7cccc(N(c8ccccc8)c8ccc(-c9ccccc9)c9ccccc89)c7)cccc6c5c4c3o2)cc1 ; c1ccc(-c2nc3c(ccc4oc5ccc6cc(-c7ccccc7N(c7ccccc7)c7cccc(-c8cccc9ccccc89)c7)ccc6c5c43)o2)cc1]

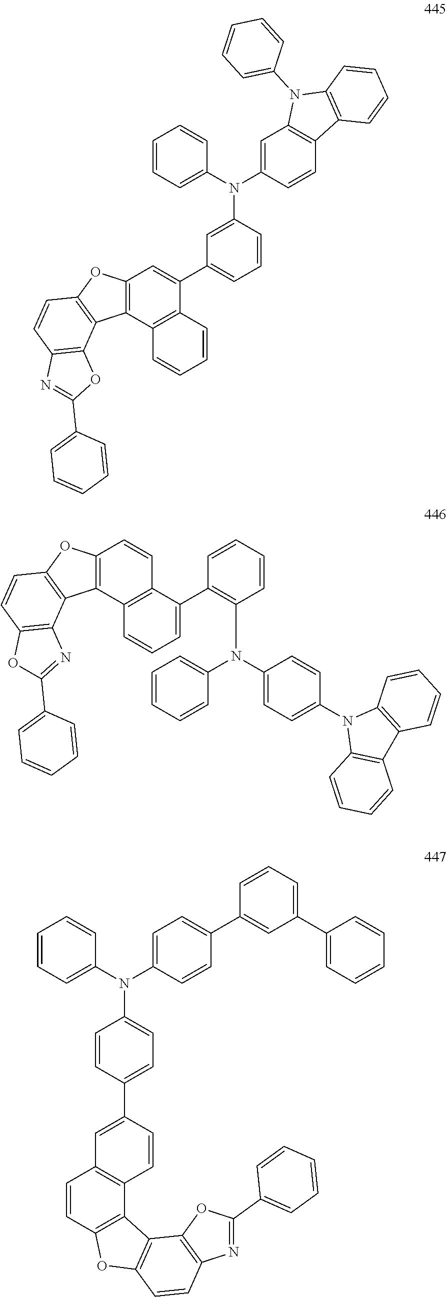 [SMILES: c1ccc(-c2nc3ccc4oc5cc(-c6cccc(N(c7ccccc7)c7ccc8c9ccccc9n(-c9ccccc9)c8c7)c6)c6ccccc6c5c4c3o2)cc1 ; c1ccc(-c2nc3c(ccc4oc5ccc6c(-c7ccccc7N(c7ccccc7)c7ccc(-n8c9ccccc9c9ccccc98)cc7)cccc6c5c43)o2)cc1 ; c1ccc(-c2cccc(-c3ccc(N(c4ccccc4)c4ccc(-c5ccc6c(ccc7oc8ccc9nc(-c%10ccccc%10)oc9c8c76)c5)cc4)cc3)c2)cc1]

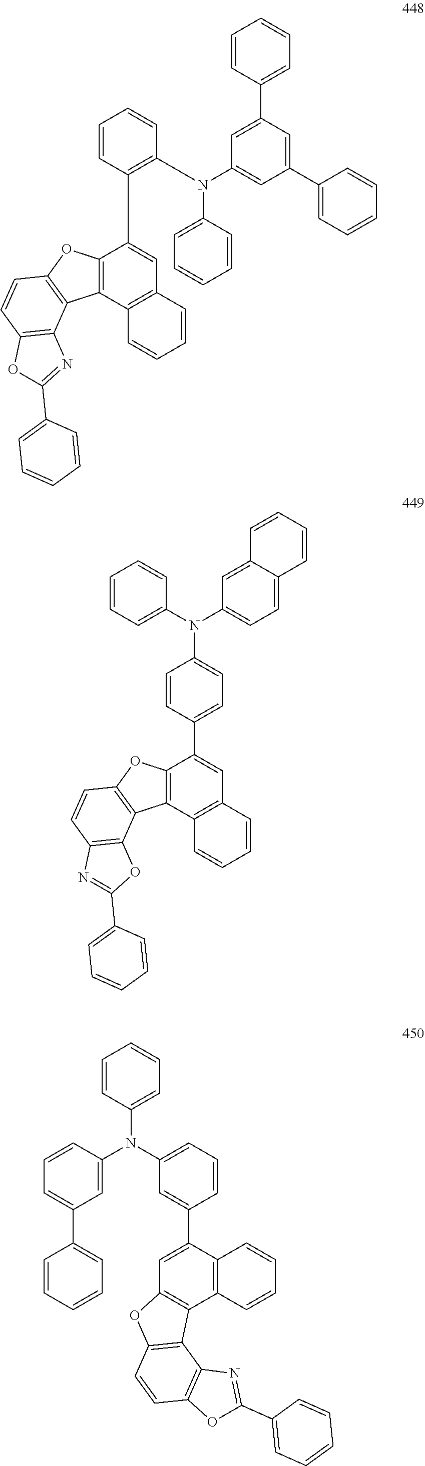 [SMILES: c1ccc(-c2cc(-c3ccccc3)cc(N(c3ccccc3)c3ccccc3-c3cc4ccccc4c4c3oc3ccc5oc(-c6ccccc6)nc5c34)c2)cc1 ; c1ccc(-c2nc3ccc4oc5c(-c6ccc(N(c7ccccc7)c7ccc8ccccc8c7)cc6)cc6ccccc6c5c4c3o2)cc1 ; c1ccc(-c2cccc(N(c3ccccc3)c3cccc(-c4cc5oc6ccc7oc(-c8ccccc8)nc7c6c5c5ccccc45)c3)c2)cc1]

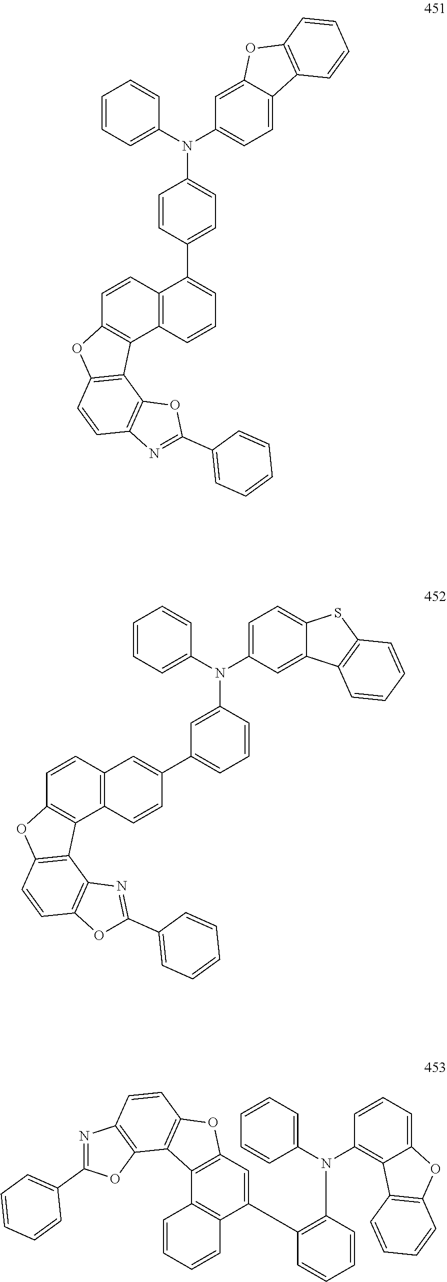 [SMILES: c1ccc(-c2nc3ccc4oc5ccc6c(-c7ccc(N(c8ccccc8)c8ccc9c(c8)oc8ccccc89)cc7)cccc6c5c4c3o2)cc1 ; c1ccc(-c2nc3c(ccc4oc5ccc6cc(-c7cccc(N(c8ccccc8)c8ccc9sc%10ccccc%10c9c8)c7)ccc6c5c43)o2)cc1 ; c1ccc(-c2nc3ccc4oc5cc(-c6ccccc6N(c6ccccc6)c6cccc7oc8ccccc8c67)c6ccccc6c5c4c3o2)cc1]

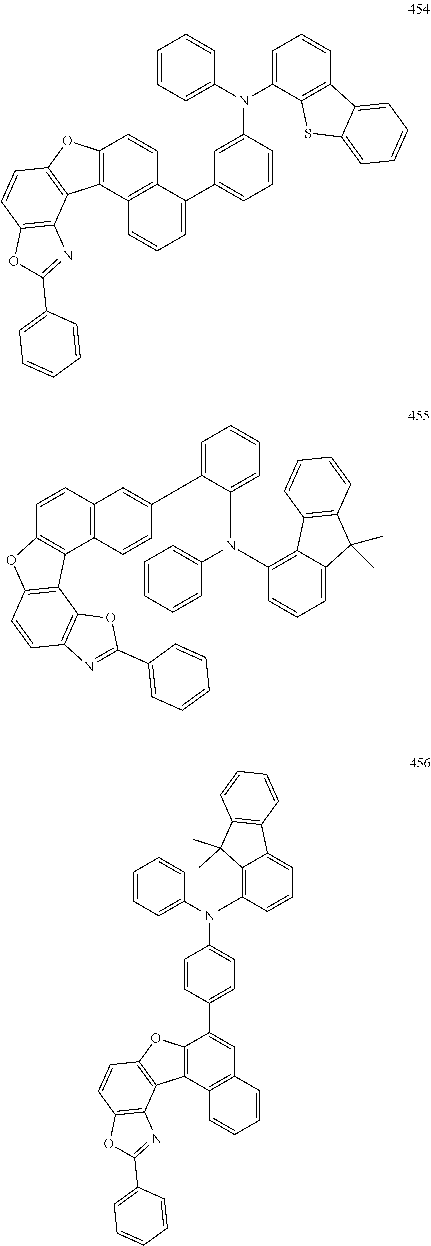 [SMILES: c1ccc(-c2nc3c(ccc4oc5ccc6c(-c7cccc(N(c8ccccc8)c8cccc9c8sc8ccccc89)c7)cccc6c5c43)o2)cc1 ; CC1(C)c2ccccc2-c2c(N(c3ccccc3)c3ccccc3-c3ccc4c(ccc5oc6ccc7nc(-c8ccccc8)oc7c6c54)c3)cccc21 ; CC1(C)c2ccccc2-c2cccc(N(c3ccccc3)c3ccc(-c4cc5ccccc5c5c4oc4ccc6oc(-c7ccccc7)nc6c45)cc3)c21]

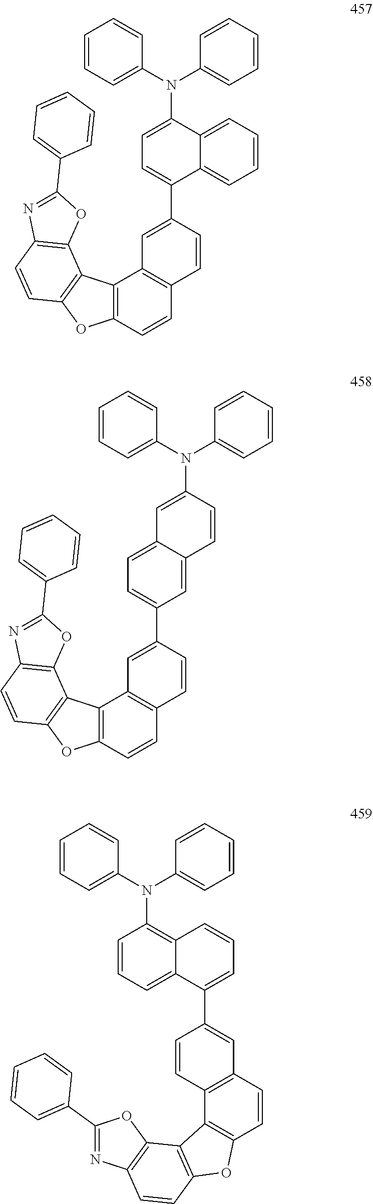 [SMILES: c1ccc(-c2nc3ccc4oc5ccc6ccc(-c7ccc(N(c8ccccc8)c8ccccc8)c8ccccc78)cc6c5c4c3o2)cc1 ; c1ccc(-c2nc3ccc4oc5ccc6ccc(-c7ccc8cc(N(c9ccccc9)c9ccccc9)ccc8c7)cc6c5c4c3o2)cc1 ; c1ccc(-c2nc3ccc4oc5ccc6cc(-c7cccc8c(N(c9ccccc9)c9ccccc9)cccc78)ccc6c5c4c3o2)cc1]

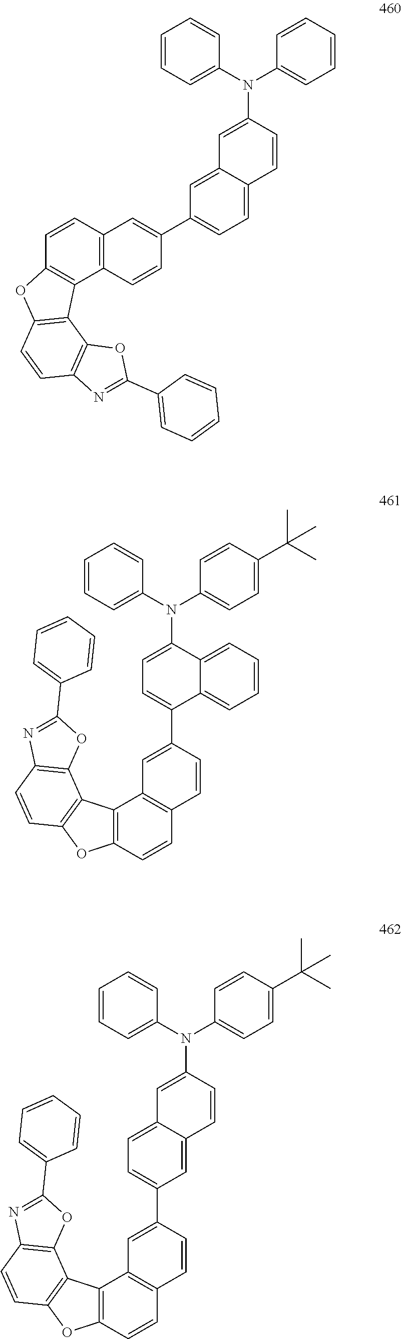 [SMILES: c1ccc(-c2nc3ccc4oc5ccc6cc(-c7ccc8ccc(N(c9ccccc9)c9ccccc9)cc8c7)ccc6c5c4c3o2)cc1 ; CC(C)(C)c1ccc(N(c2ccccc2)c2ccc(-c3ccc4ccc5oc6ccc7nc(-c8ccccc8)oc7c6c5c4c3)c3ccccc23)cc1 ; CC(C)(C)c1ccc(N(c2ccccc2)c2ccc3cc(-c4ccc5ccc6oc7ccc8nc(-c9ccccc9)oc8c7c6c5c4)ccc3c2)cc1]

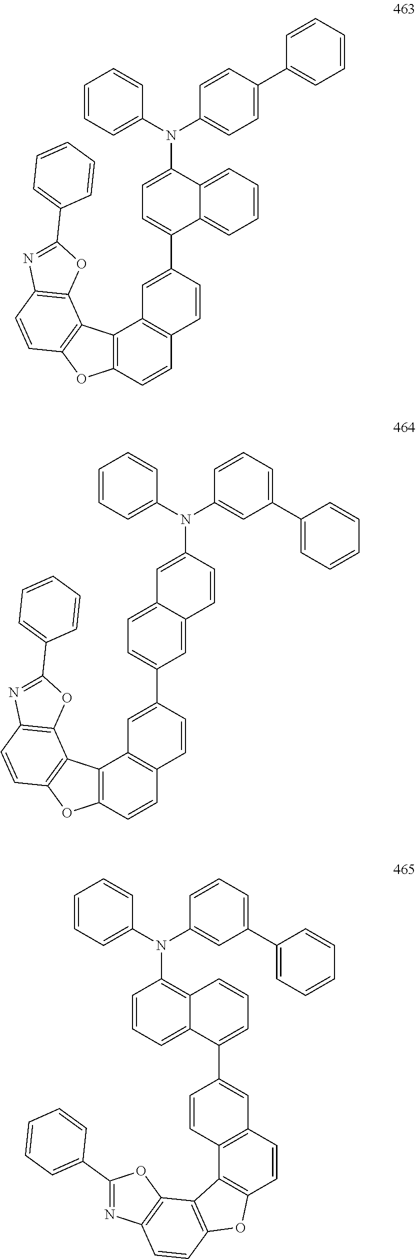 [SMILES: c1ccc(-c2ccc(N(c3ccccc3)c3ccc(-c4ccc5ccc6oc7ccc8nc(-c9ccccc9)oc8c7c6c5c4)c4ccccc34)cc2)cc1 ; c1ccc(-c2cccc(N(c3ccccc3)c3ccc4cc(-c5ccc6ccc7oc8ccc9nc(-c%10ccccc%10)oc9c8c7c6c5)ccc4c3)c2)cc1 ; c1ccc(-c2cccc(N(c3ccccc3)c3cccc4c(-c5ccc6c(ccc7oc8ccc9nc(-c%10ccccc%10)oc9c8c76)c5)cccc34)c2)cc1]

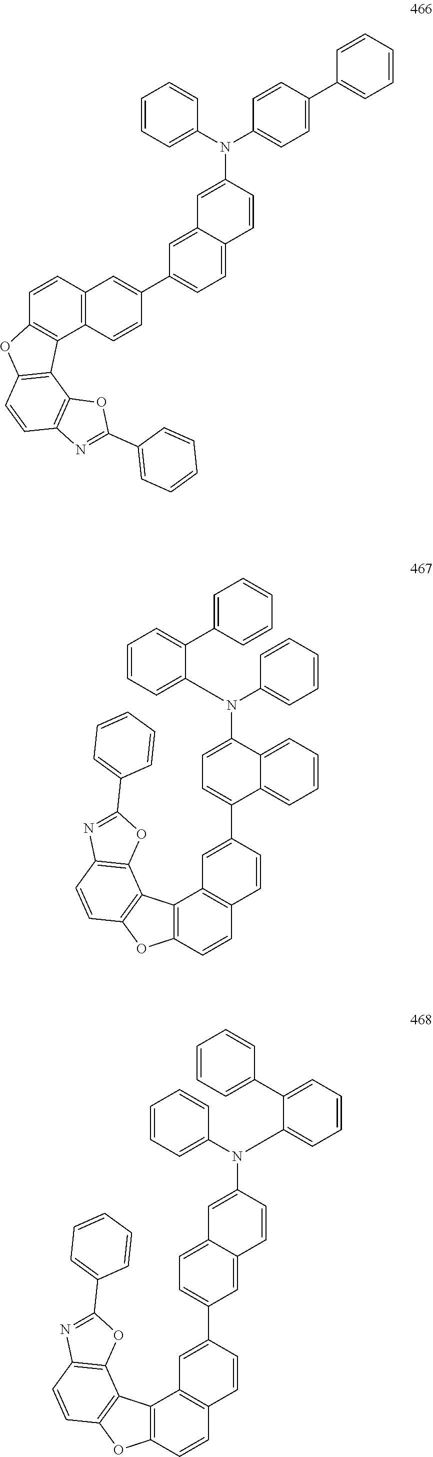 [SMILES: c1ccc(-c2ccc(N(c3ccccc3)c3ccc4ccc(-c5ccc6c(ccc7oc8ccc9nc(-c%10ccccc%10)oc9c8c76)c5)cc4c3)cc2)cc1 ; c1ccc(-c2nc3ccc4oc5ccc6ccc(-c7ccc(N(c8ccccc8)c8ccccc8-c8ccccc8)c8ccccc78)cc6c5c4c3o2)cc1 ; c1ccc(-c2nc3ccc4oc5ccc6ccc(-c7ccc8cc(N(c9ccccc9)c9ccccc9-c9ccccc9)ccc8c7)cc6c5c4c3o2)cc1]

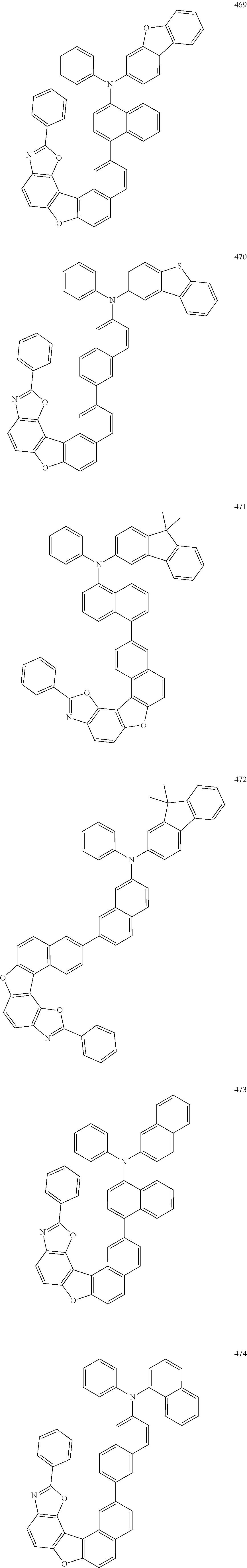

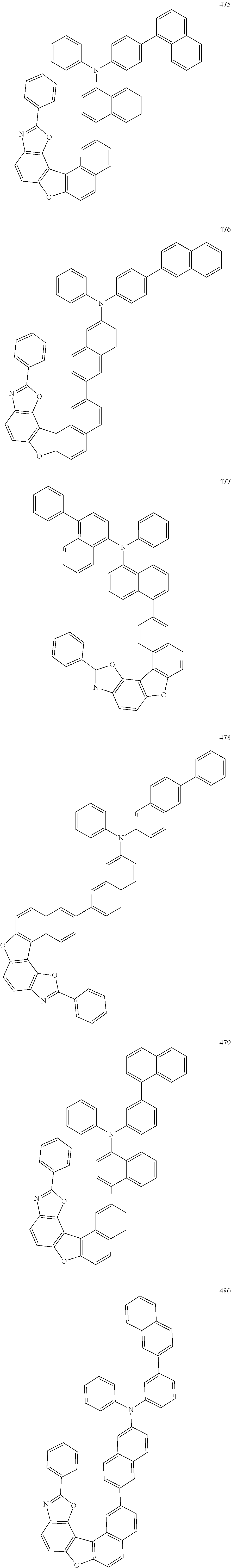

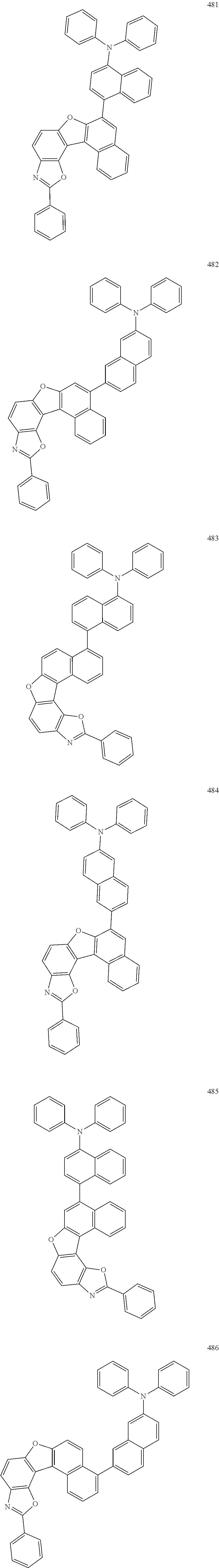 [SMILES: c1ccc(-c2nc3ccc4oc5c(-c6ccc(N(c7ccccc7)c7ccccc7)c7ccccc67)cc6ccccc6c5c4c3o2)cc1 ; c1ccc(-c2nc3ccc4oc5cc(-c6ccc7ccc(N(c8ccccc8)c8ccccc8)cc7c6)c6ccccc6c5c4c3o2)cc1 ; c1ccc(-c2nc3ccc4oc5ccc6c(-c7cccc8c(N(c9ccccc9)c9ccccc9)cccc78)cccc6c5c4c3o2)cc1 ; c1ccc(-c2nc3ccc4oc5c(-c6ccc7cc(N(c8ccccc8)c8ccccc8)ccc7c6)cc6ccccc6c5c4c3o2)cc1 ; c1ccc(-c2nc3ccc4oc5cc(-c6ccc(N(c7ccccc7)c7ccccc7)c7ccccc67)c6ccccc6c5c4c3o2)cc1 ; c1ccc(-c2nc3ccc4oc5ccc6c(-c7ccc8ccc(N(c9ccccc9)c9ccccc9)cc8c7)cccc6c5c4c3o2)cc1]

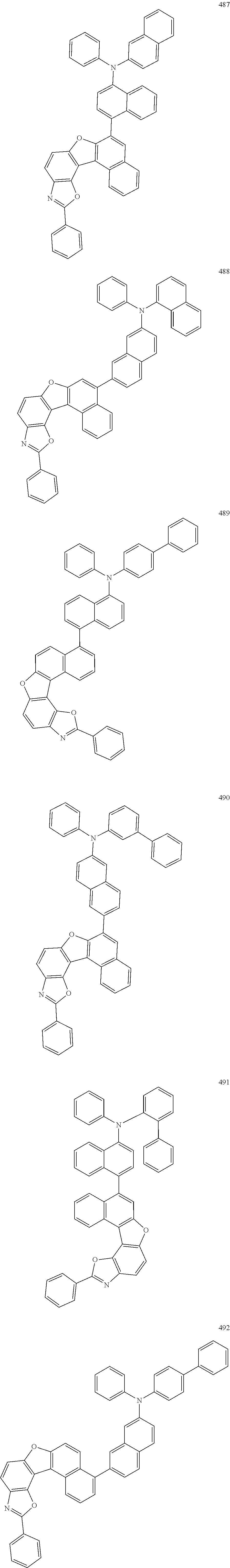

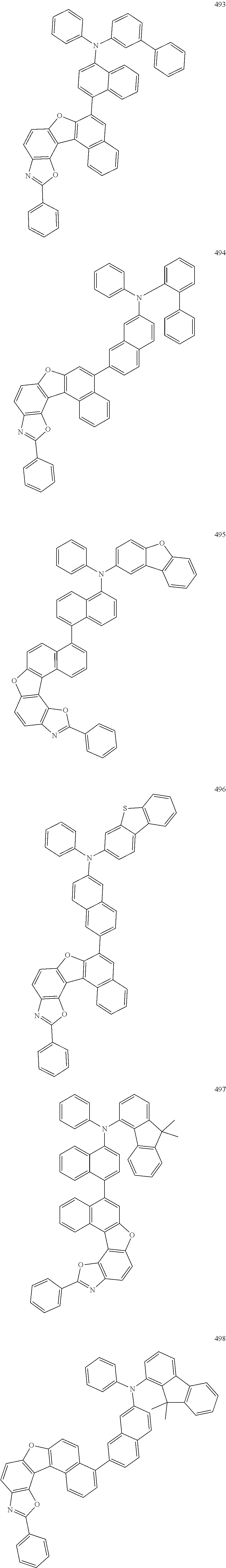

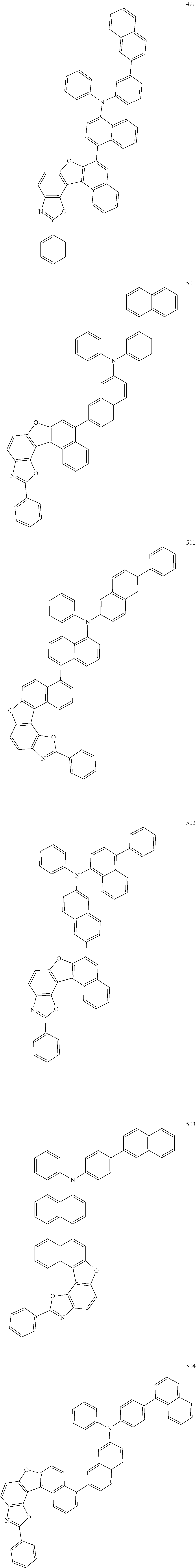

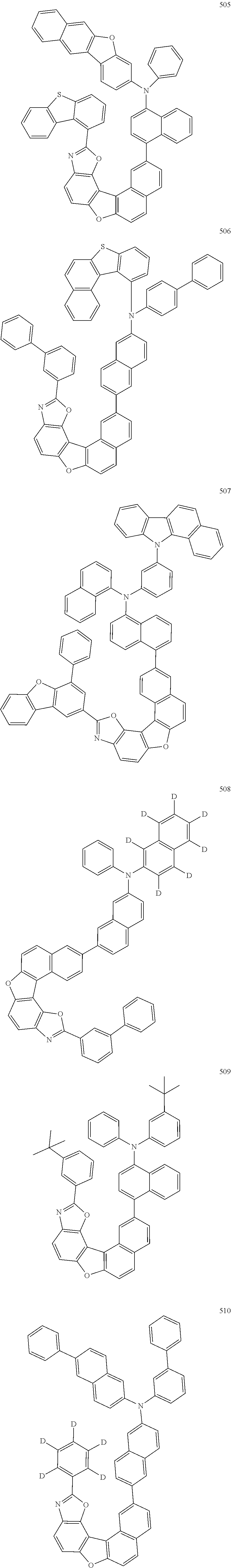

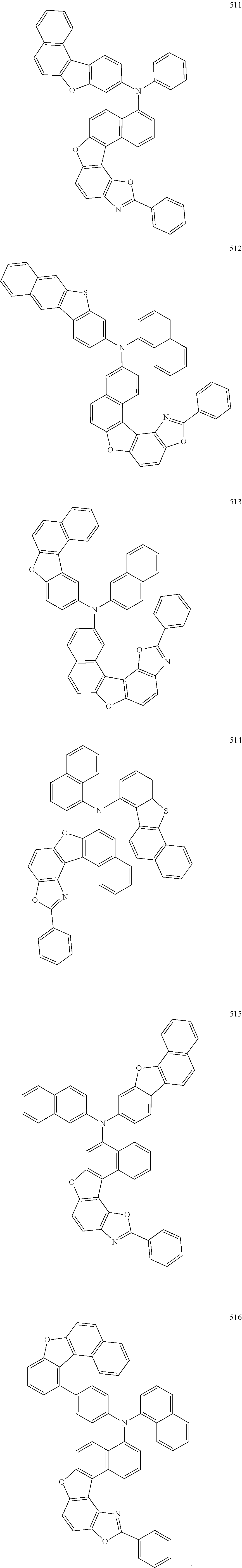

In a second aspect, the present application provides an organic electroluminescent device, comprising an anode, a cathode, and a functional layer disposed between the anode and the cathode, where the functional layer includes the heterocyclic compound described in the first aspect of the present application.

The heterocyclic compound provided in the present application may be used for forming at least one organic film layer in the functional layer to improve the luminous efficiency, service life, and other characteristics of the organic electroluminescent device.

Optionally, the functional layer comprises an organic luminescent layer, and the organic luminescent layer includes the heterocyclic compound. The organic luminescent layer may be formed by the heterocyclic compound provided in the present application, or the heterocyclic compound provided in the present application and other materials.

Optionally, the functional layer further includes a hole transport layer and a hole adjustment layer, the hole transport layer is disposed between the anode and the organic luminescent layer, and the hole adjustment layer is disposed between the hole transport layer and the organic luminescent layer. In some embodiments, the hole adjustment layer is formed by the heterocyclic compound provided in the present application, or the heterocyclic compound provided in the present application and other materials.

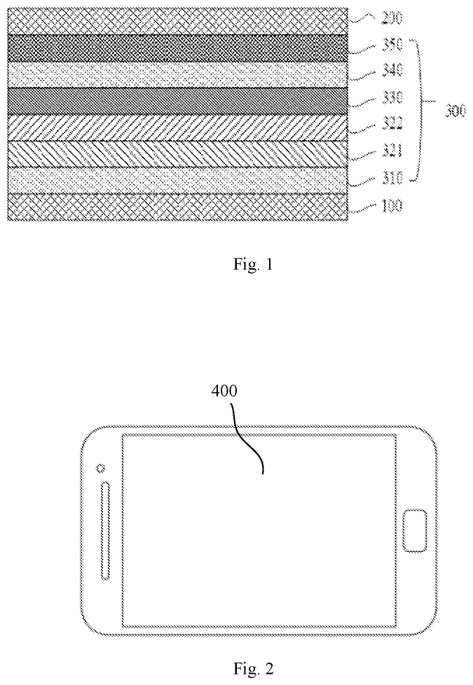

According to a specific embodiment, the organic electroluminescent device, as shown in FIG. 1, includes an anode 100, a hole injection layer 310, a hole transport layer 321, a hole adjustment layer (also referred to as a hole auxiliary layer) 322, an organic luminescent layer 330, an electron transport layer 340, an electron injection layer 350, and a cathode 200 stacked in sequence.

In the present application, the anode 100 includes an anode material, preferably a material with a large work function that facilitates hole injection into the functional layer. Specific examples of the anode material include, but are not limited to: metals such as nickel, platinum, vanadium, chromium, copper, zinc, and gold, or alloys thereof, metal oxides such as zinc oxide, indium oxide, indium tin oxide (ITO), and indium zinc oxide (IZO); combined metals and oxides such as ZnO:Al or SnO₂:Sb; or conductive polymers such as poly(3-methylthiophene), poly[3,4-(ethylidene-1,2-dioxy)thiophene] (PEDT), polypyrrole, and polyaniline. Preferably, a transparent electrode containing indium tin oxide (ITO) as the anode is included.

In the present application, the hole transport layer or hole adjustment layer may include one or more hole transport materials, and the materials for the hole transport layer may be selected from carbazole polymers, carbazole linked triarylamine compounds, or other types of compounds, specifically from compounds shown below or any combination thereof:

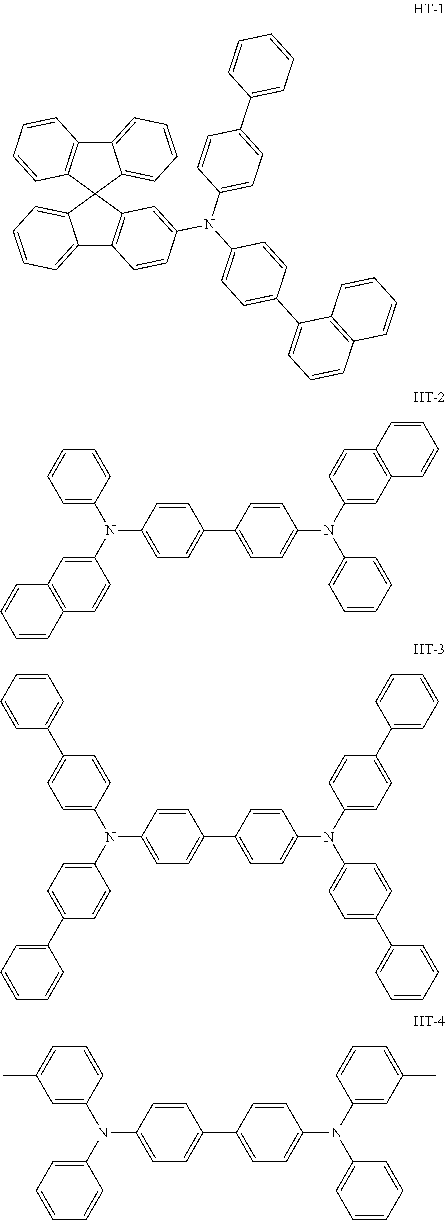

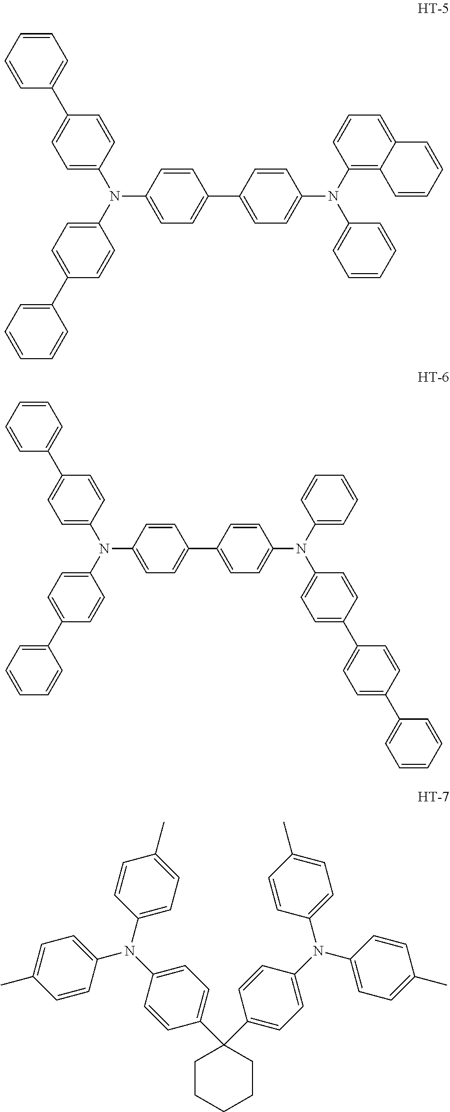

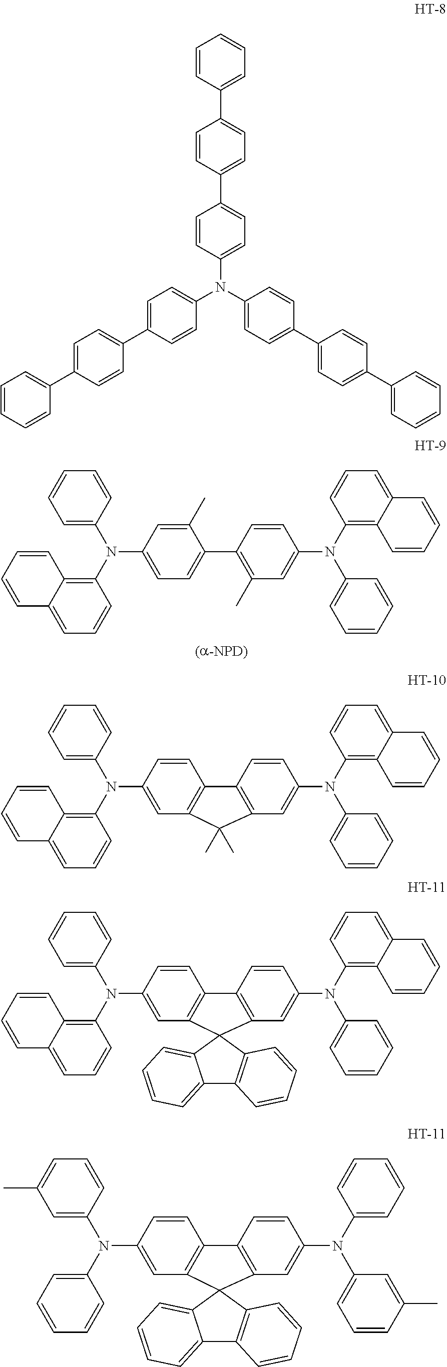

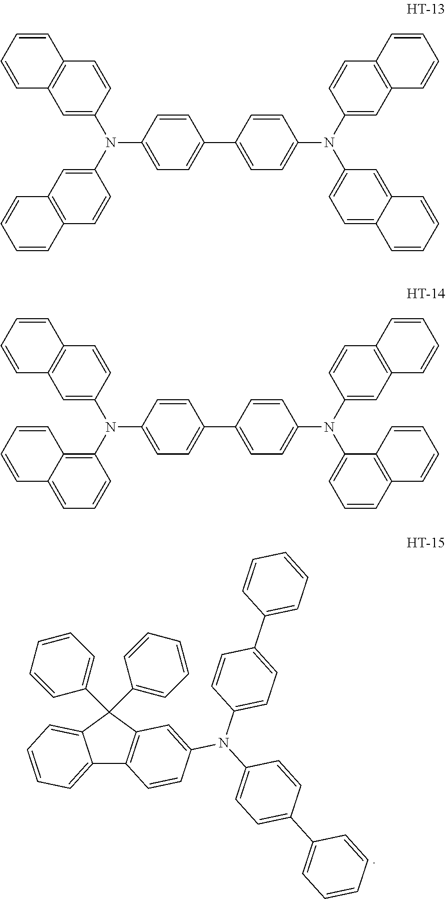 [SMILES: c1ccc2cc(N(c3ccc(-c4ccc(N(c5ccc6ccccc6c5)c5ccc6ccccc6c5)cc4)cc3)c3ccc4ccccc4c3)ccc2c1 ; c1ccc2cc(N(c3ccc(-c4ccc(N(c5ccc6ccccc6c5)c5cccc6ccccc56)cc4)cc3)c3cccc4ccccc34)ccc2c1 ; c1ccc(-c2ccc(N(c3ccc(-c4ccccc4)cc3)c3ccc4c(c3)C(c3ccccc3)(c3ccccc3)c3ccccc3-4)cc2)cc1]

In one embodiment, the hole transport layer 321 may be formed by α-NPD.

In one embodiment, the hole adjustment layer 322 is formed by HT-1.

In one embodiment of the present application, the hole adjustment layer 322 is formed by the heterocyclic compound of the present application.

Optionally, the hole injection layer 310 is further disposed between the anode 100 and the hole transport layer 321 to enhance the ability to inject holes into the hole transport layer 321. The hole injection layer 310 may be made of a benzidine derivative, a starburst arylamine compound, a phthalocyanine derivative, or other materials, and the present application does not make special restrictions on this. A material for the hole injection layer 310 may be, for example, selected from the following compounds or any combination thereof:

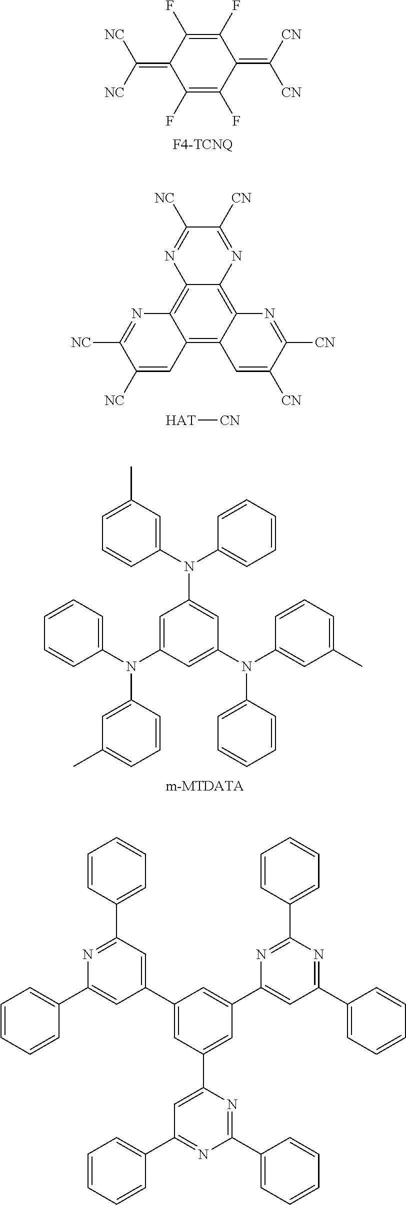

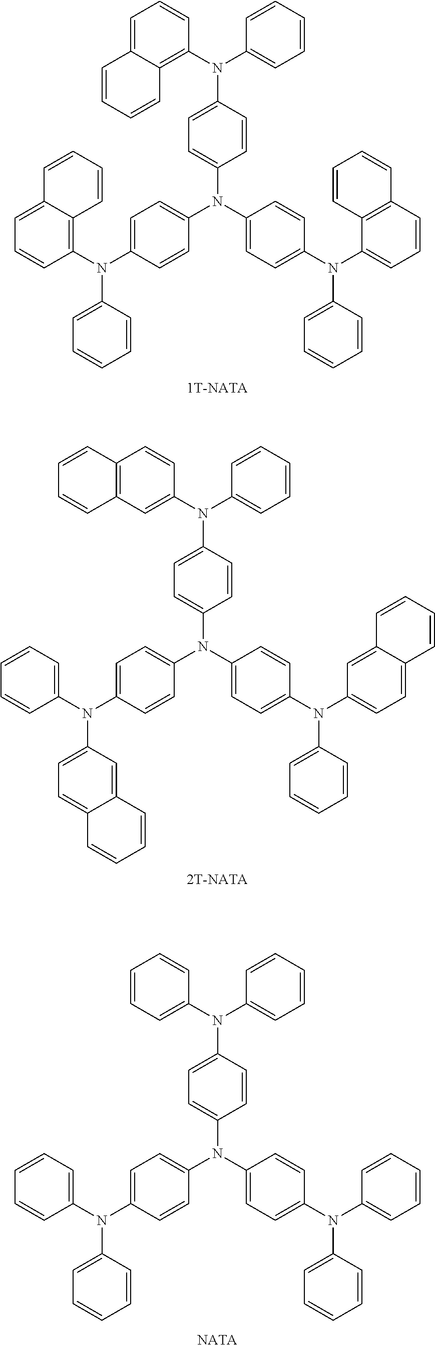

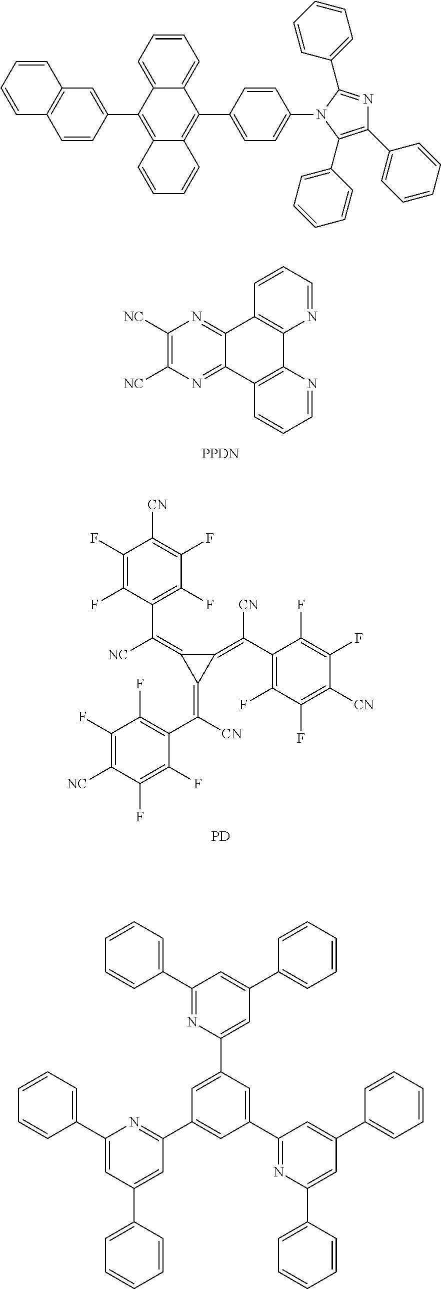 [SMILES: c1ccc(-c2nc(-c3ccccc3)n(-c3ccc(-c4c5ccccc5c(-c5ccc6ccccc6c5)c5ccccc45)cc3)c2-c2ccccc2)cc1 ; N#Cc1nc2c3cccnc3c3ncccc3c2nc1C#N ; "PPDN" ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; "PD" ; c1ccc(-c2cc(-c3ccccc3)nc(-c3cc(-c4cc(-c5ccccc5)cc(-c5ccccc5)n4)cc(-c4cc(-c5ccccc5)cc(-c5ccccc5)n4)c3)c2)cc1]

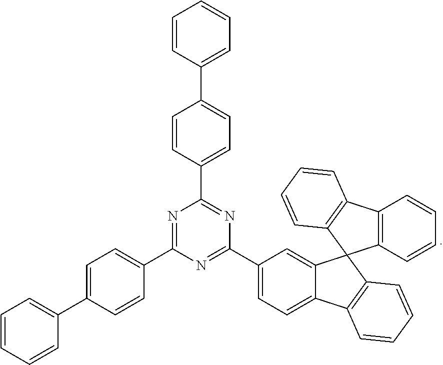 [SMILES: c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1]

In one embodiment of the present application, the hole injection layer 310 is formed by PD.

Optionally, the organic luminescent layer 330 may be formed by a single luminescent material or include a host material and a guest material. Optionally, the organic luminescent layer 330 includes a host material and a guest material, holes injected into the organic luminescent layer 330 and electrons injected into the organic luminescent layer 330 may recombine in the organic luminescent layer 330 to form excitons, the excitons transfer energy to the host material, the host material transfers the energy to the guest material, and the guest material is enabled to emit light.

The host material of the organic luminescent layer 330 may include metal chelating compounds, bisphenylvinyl derivatives, aromatic amine derivatives, dibenzofuranyl derivatives, or other types of materials. The host material of the organic luminescent layer 330 may be a compound, or a combination of two or more compounds. Optionally, the host material includes the heterocyclic compound of the present application.

The guest material of the organic luminescent layer 330 may be a compound with a condensed aromatic ring or a derivative thereof, a compound with a heteroaromatic ring or a derivative thereof, an aromatic amine derivative, or other materials, and the present application does not make special restrictions on this. The guest material is also referred to as a doping material or dopant. According to a type of luminescence, the dopant may be a fluorescent dopant or a phosphorescent dopant. For example, specific examples of the phosphorescent dopant include, but are not limited to,

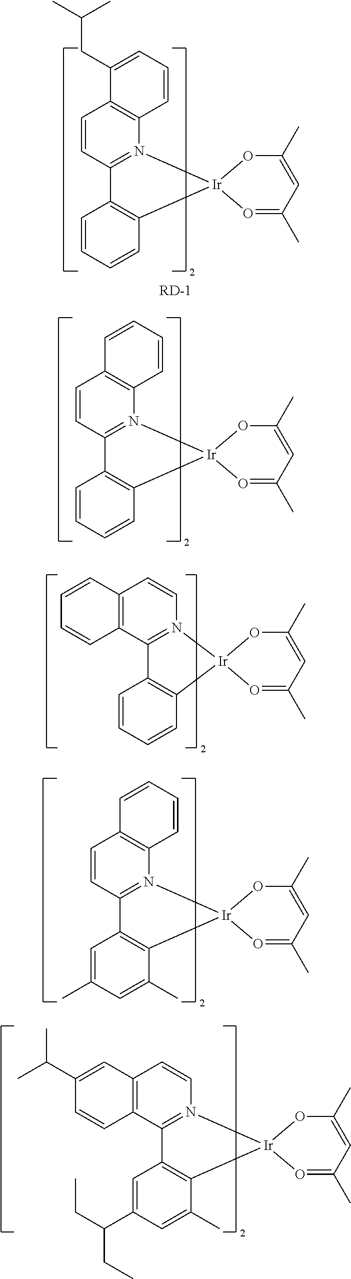

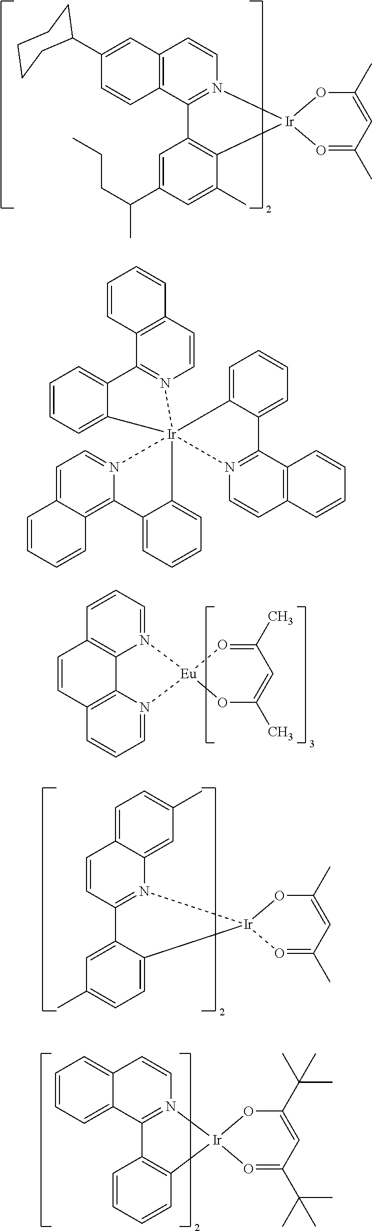

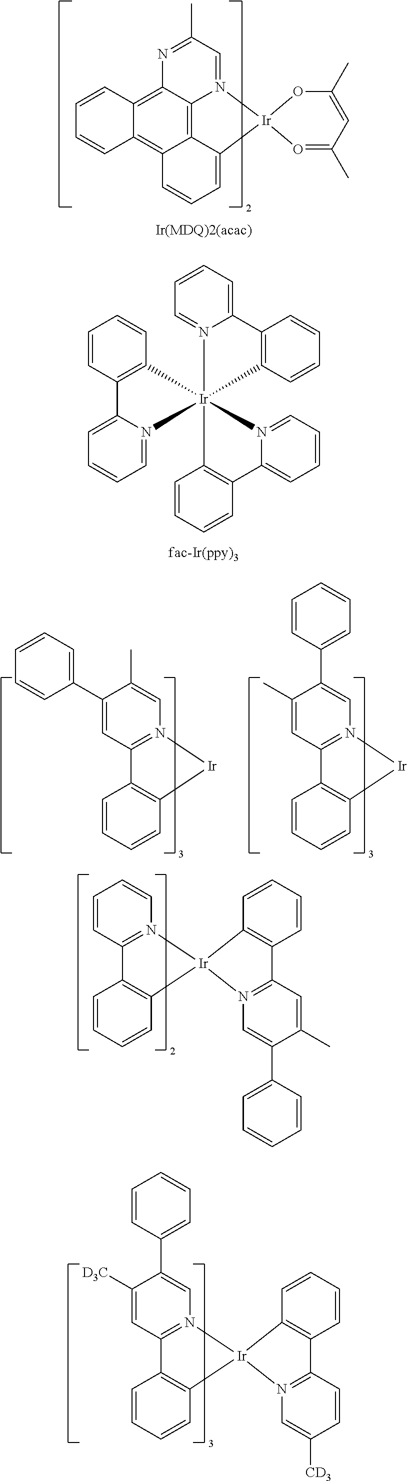

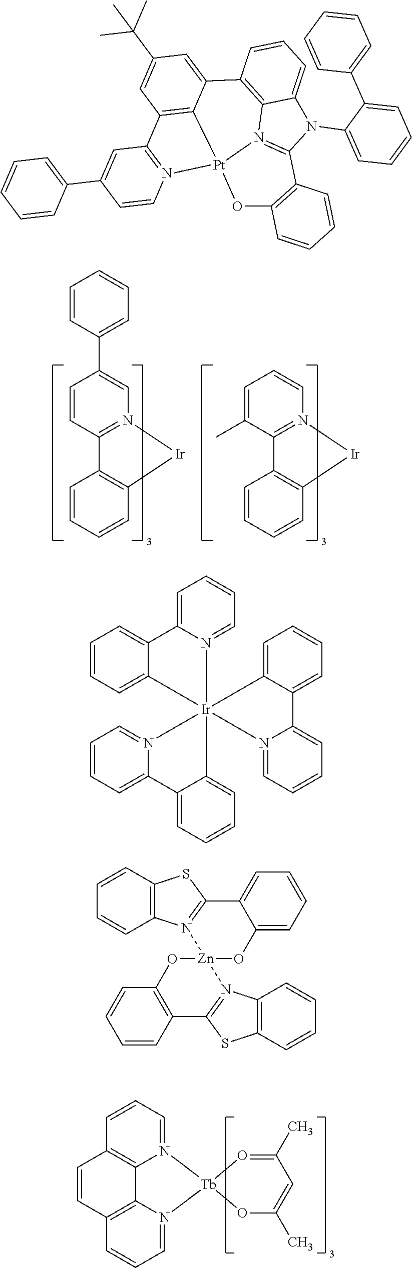

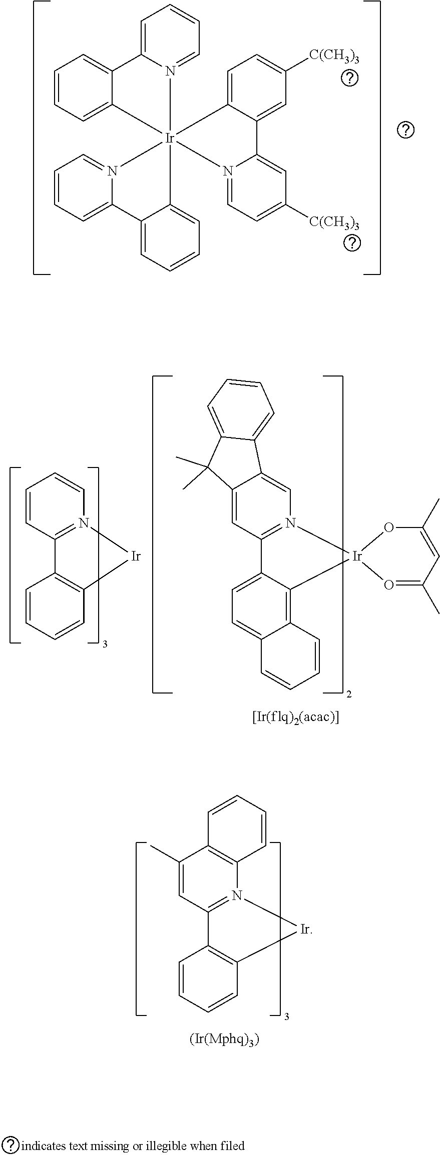

In one embodiment of the present application, the organic electroluminescent device is a red organic electroluminescent device. In a more specific embodiment, the host material of the organic luminescent layer 330 includes the heterocyclic compound of the present application. The guest material may be, for example, RD-1.

In another embodiment, the organic electroluminescent device is a green organic electroluminescent device. In a more specific embodiment, the host material of the organic luminescent layer 330 includes the heterocyclic compound of the present application. The guest material may be, for example, fac-Ir(ppy)₃.

The electron transport layer 340 may be of a single-layer structure or a multi-layer structure and may include one or more electron transport materials, and the electron transport materials may be selected from, but not limited to, ET-1, BmPyPhB, LiQ, benzimidazole derivatives, oxadiazole derivatives, quinoxaline derivatives, or other electron transport materials, which are not specifically limited in the present application. A material for the electron transport layer 340 includes, but is not limited to, the following compounds:

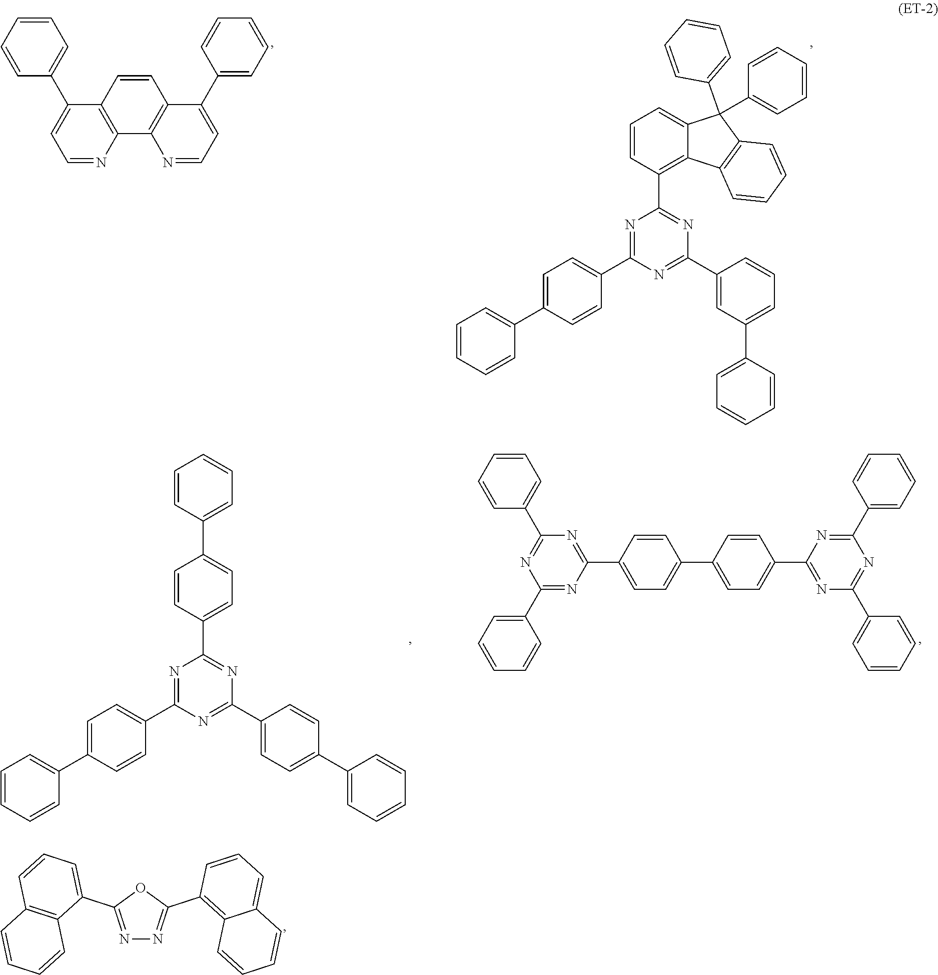

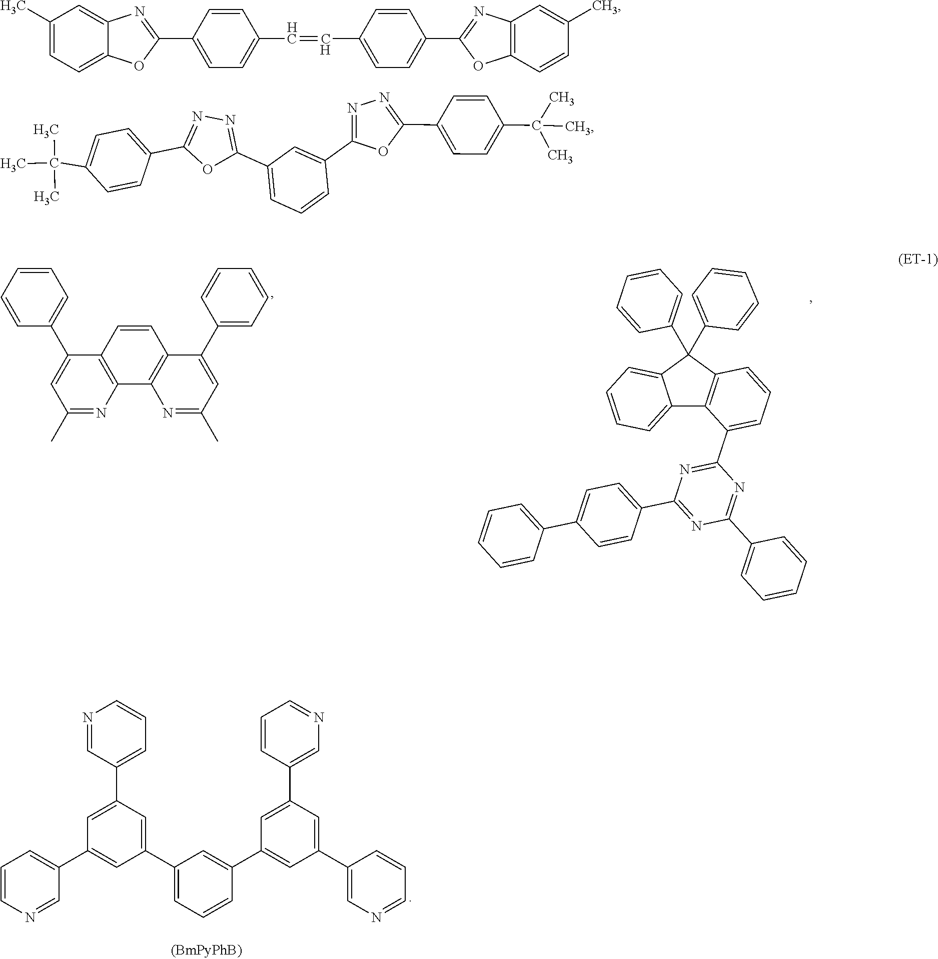

In one embodiment of the present application, the electron transport layer 340 is formed by ET-1 and LiQ.

In the present application, the cathode 200 includes a cathode material, which is a material with a small work function that facilitates electron injection into the functional layer. Specific examples of the cathode material include, but are not limited to, metals such as magnesium, calcium, sodium, potassium, titanium, indium, yttrium, lithium, gadolinium, aluminum, silver, tin, and lead, or alloys thereof, or multi-layer materials such as LiF/Al, Liq/Al, LiO₂/Al, LiF/Ca, LiF/Al, and BaF₂/Ca. Optionally, a metal electrode containing magnesium and silver is used as the cathode.

Optionally, the electron injection layer 350 is further disposed between the cathode 200 and the electron transport layer 340 to enhance the ability to inject electrons into the electron transport layer 340. The electron injection layer 350 may include inorganic materials such as alkali metal sulfides and alkali metal halides, or may include complexes of alkali metals and organic compounds. In one embodiment of the present application, the electron injection layer 350 includes ytterbium (Yb).

A third aspect of the present application provides an electronic apparatus, comprising the organic electroluminescent device described in the second aspect of the present application.

According to an embodiment, as shown in FIG. 2, the provided electronic apparatus is an electronic apparatus 400, which includes the foregoing organic electroluminescent device. The electronic apparatus 400 may be, for example, a display apparatus, a lighting apparatus, an optical communication apparatus, or other types of electronic apparatuses, including but not limited to a computer screen, a mobile phone screen, a television, an electronic paper, an emergency light, or an optical module.

A synthesis method of the heterocyclic compound in the present application is specifically described below in combination with a synthesis example, but the present application is not subject to any restriction.

### SYNTHESIS EXAMPLES

Those skilled in the art should recognize that chemical reactions described in the present application may be used for properly preparing many heterocyclic compounds in the present application, and other methods for preparing the compounds in the present application are considered to fall within the scope of the present application. For example, according to the present application, the synthesis of non-exemplary compounds may be successfully completed by those skilled in the art through modification methods, such as appropriate protection of interfering groups, use of other known reagents in addition to those described in the present application, or some conventional modifications to reaction conditions. Compounds for which synthesis methods are not mentioned in the present application are all raw materials obtained through commercial channels.

### Synthesis of 7-bromo-1-iodo-2 naphthalenethiol

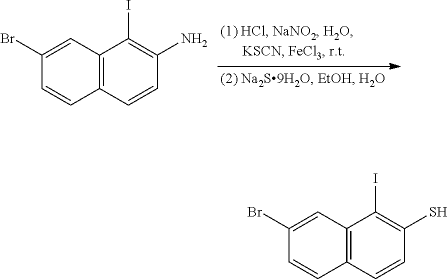 [SMILES: Nc1ccc2ccc(Br)cc2c1I ; Sc1ccc2ccc(Br)cc2c1I]

Under nitrogen atmosphere, 7-bromo-1-iodo-2-naphthylamine (17.40 g, 50 mmol), concentrated hydrochloric acid (25 mL) and deionized water (25 mL) were sequentially added into a 1000 mL of three-necked flask, the system was cooled to 0° C. with an ice water bath, an aqueous solution (25 mL) of sodium nitrate (3.45 g, 50 mmol) was added dropwise into the system, then an aqueous solution (25 mL) of potassium thiocyanate (9.72 g, 100 mmol) and ferric chloride (4.1 g, 25 mmol) were added dropwise into the reaction system, and the system was slowly heated to room temperature and stirred for reaction overnight. The reaction solution was poured into deionized water (200 mL), the solution was extracted with dichloromethane (100 mL×3 times), organic phases were combined and dried with anhydrous sodium sulfate, filtrated, and the filtrate was distilled in vacuum to remove solvent and obtain a crude product, and the crude product was directly used for next reaction without purification.

Under nitrogen atmosphere, the obtained crude product, sodium sulfide nonahydrate (9.61 g, 100 mmol), ethanol (180 mL), and deionized water (360 mL) were added into the 1000 mL of three-necked flask at one time, heated until reflux, and stirred for reaction for 16 h. The reaction system was cooled to room temperature and then filtered, the filtrate was acidified with 1M hydrochloric acid to pH=2 and then extracted with dichloromethane (100 mL×3 times), organic phases were combined and dried with anhydrous sodium sulfate, filtrated, and the filtrate was distilled in vacuum to remove solvent and obtain a crude product; and the crude product was purified by silica gel column chromatography with n-heptane as a mobile phase to obtain a white solid, namely, 7-bromo-1-iodo-2 naphthalenethiol (8.03 g, yield 44%).

### Synthesis of Sub-a1:

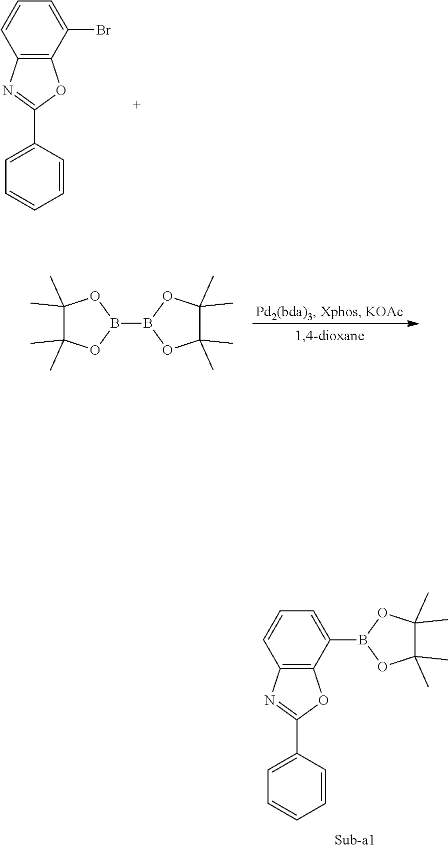

Under nitrogen atmosphere, 7-bromo-2-phenylbenzoxazole (12.06 g, 44 mmol), bis(pinacolato)diboron (12.28 g, 48.4 mmol), potassium acetate (9.50 g, 96.8 mmol), and 1,4-dioxane (120 mL) were sequentially added into a 500 mL of three-necked flask, stirring and heating were performed, tri(dibenzalacetone)dipalladium (Pd₂(dba)₃, 0.40 g, 0.44 mmol) and 2-dicyclohexylphosphine-2′,4′,6′-triisopropylbiphenyl (XPhos, 0.42 g, 0.88 mmol) were quickly added when the system was heated to 40° C., the heating was continued until reflux, and the system was stirred for reaction overnight. After the system was cooled to room temperature, 200 mL of water was added to the system, the system was stirred thoroughly for 30 min and filtered under reduced pressure, filter cakes were washed with deionized water to neutral and then rinsed with 100 mL of anhydrous ethanol, and the filter cakes were collected to obtain a crude product as a gray solid; the crude product was beaten once with n-heptane and then dissolved in 200 mL of toluene before passing through a silica gel column to remove the catalyst, and Sub-a1 as a white solid (10.17 g, yield 72%) was obtained after concentration.

With reference to the synthesis of Sub-a1, 7-bromo-2-phenylbenzoxazole was replaced by Reactant A as shown in Table 1 to synthesize Sub-a2 to Sub-a4.

TABLE 1
Synthesis of Sub-a2 to Sub-a4

| Sub-a No. | Reactant A | Structure of Sub-a | Yield (%) |
|---|---|---|---|
| Sub-a2 | 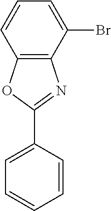 [SMILES: Brc1cccc2oc(-c3ccccc3)nc12] CAS: 17928993-81-7 | 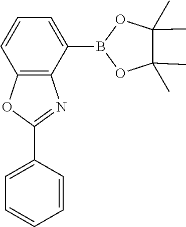 [SMILES: CC1(C)OB(c2cccc3oc(-c4ccccc4)nc23)OC1(C)C] | 64 |
| Sub-a3 | 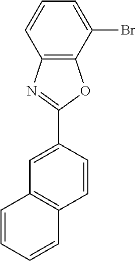 [SMILES: Brc1cccc2nc(-c3ccc4ccccc4c3)oc12] CAS: 2749511-90-6 | 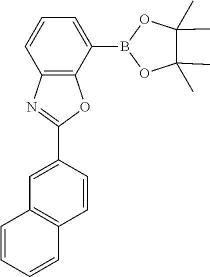 [SMILES: CC1(C)OB(c2cccc3nc(-c4ccc5ccccc5c4)oc23)OC1(C)C] | 66 |
| Sub-a4 | 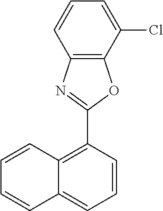 [SMILES: Clc1cccc2nc(-c3cccc4ccccc34)oc12] CAS: 2415412-21-2 | 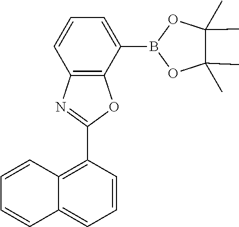 [SMILES: CC1(C)OB(c2cccc3nc(-c4cccc5ccccc45)oc23)OC1(C)C] | 62 |

### Synthesis of Sub-b1:

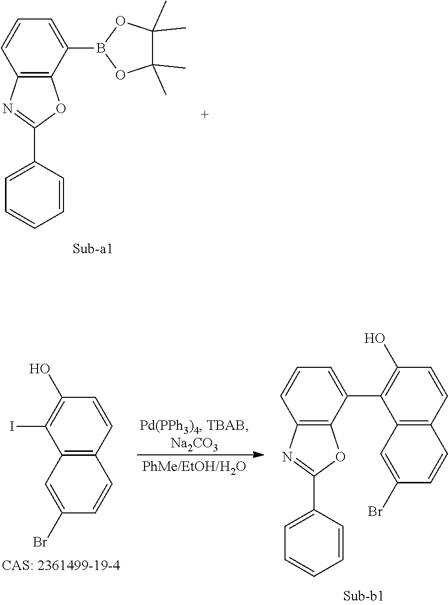 [SMILES: CC1(C)OB(c2cccc3nc(-c4ccccc4)oc23)OC1(C)C ; Oc1ccc2ccc(Br)cc2c1I ; Oc1ccc2ccc(Br)cc2c1-c1cccc2nc(-c3ccccc3)oc12]

Under nitrogen atmosphere, Sub-a1 (17.66 g, 55 mmol), 7-bromo-1-iodo-2-hydroxynaphthalene (17.45 g, 50 mmol), tetra (triphenylphosphine) palladium (Pd(PPh₃)₄, 0.58 g, 0.5 mmol), anhydrous sodium carbonate (10.60 g, 100 mmol), toluene (180 mL), anhydrous ethanol (45 mL), and deionized water (45 mL) were sequentially added into a 500 mL of three-necked flask, stirring and heating until reflux were performed, and the system reacted for 16 h. The system was cooled to room temperature and then extracted with dichloromethane (150 mL×3 times), organic phases were combined and dried with anhydrous magnesium sulfate, filtrated, and the filtrate was distilled in vacuum to remove solvent, and a crude product was obtained. The crude product was purified by silica gel column chromatography with n-heptane as a mobile phase to obtain Sub-b1 as a white solid (11.03 g, yield 53%).

With reference to the synthesis of Sub-b1, Sub-a1 was replaced by Reactant B as shown in Table 2, and 7-bromo-1-iodo-2-hydroxynaphthalene was replaced by Reactant C to synthesize Sub-b2 to Sub-b11.

TABLE 2
Synthesis of Sub-b2 to Sub-b11

| Sub-b No. | Reactant B | Reactant C | Structure of Sub-b | Yield (%) |
|---|---|---|---|---|
| Sub-b2 | 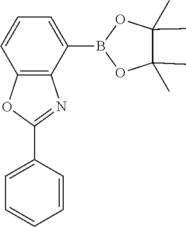 [SMILES: CC1(C)OB(c2cccc3oc(-c4ccccc4)nc23)OC1(C)C] | 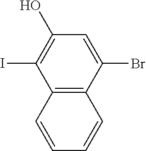 [SMILES: Oc1cc(Br)c2ccccc2c1I] CAS: 2364362-34-3 | 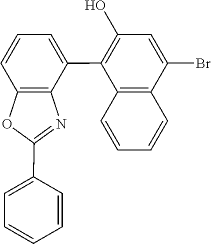 [SMILES: Oc1cc(Br)c2ccccc2c1-c1cccc2oc(-c3ccccc3)nc12] | 46 |
| Sub-b3 | 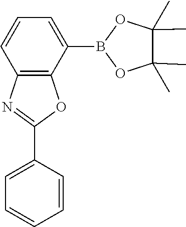 [SMILES: CC1(C)OB(c2cccc3nc(-c4ccccc4)oc23)OC1(C)C] | 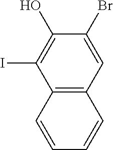 [SMILES: Oc1c(Br)cc2ccccc2c1I] CAS: 102154-09-6 | 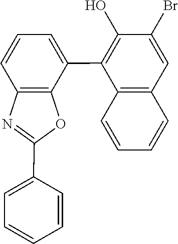 [SMILES: Oc1c(Br)cc2ccccc2c1-c1cccc2nc(-c3ccccc3)oc12] | 55 |
| Sub-b4 | 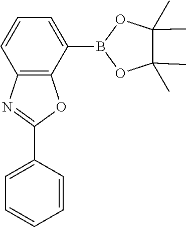 [SMILES: CC1(C)OB(c2cccc3nc(-c4ccccc4)oc23)OC1(C)C] | 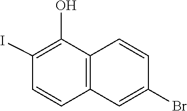 [SMILES: Oc1c(I)ccc2cc(Br)ccc12] CAS: 2364360-82-5 | 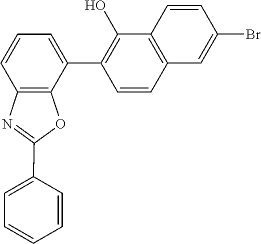 [SMILES: Oc1c(-c2cccc3nc(-c4ccccc4)oc23)ccc2cc(Br)ccc12] | 52 |
| Sub-b5 | 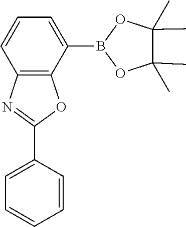 [SMILES: CC1(C)OB(c2cccc3nc(-c4ccccc4)oc23)OC1(C)C] | 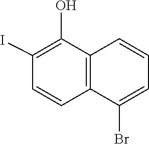 [SMILES: Oc1c(I)ccc2c(Br)cccc12] CAS: 2364362-20-7 | 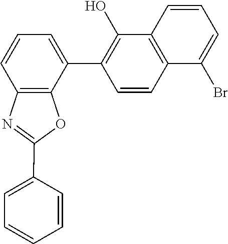 [SMILES: Oc1c(-c2cccc3nc(-c4ccccc4)oc23)ccc2c(Br)cccc12] | 51 |
| Sub-b6 | 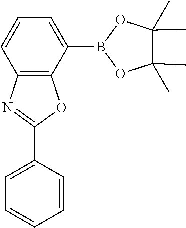 [SMILES: CC1(C)OB(c2cccc3nc(-c4ccccc4)oc23)OC1(C)C] | 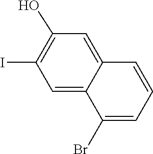 [SMILES: Oc1cc2cccc(Br)c2cc1I] CAS: 2364362-33-2 | 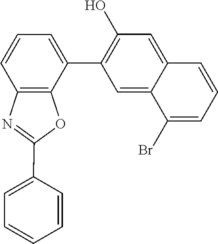 [SMILES: Oc1cc2cccc(Br)c2cc1-c1cccc2nc(-c3ccccc3)oc12] | 49 |
| Sub-b7 | 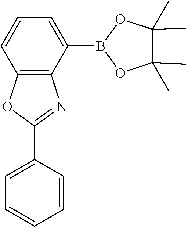 [SMILES: CC1(C)OB(c2cccc3oc(-c4ccccc4)nc23)OC1(C)C] | 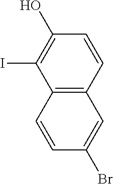 [SMILES: Oc1ccc2cc(Br)ccc2c1I] CAS: 102153-45-7 | 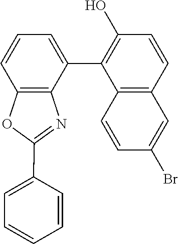 [SMILES: Oc1ccc2cc(Br)ccc2c1-c1cccc2oc(-c3ccccc3)nc12] | 51 |
| Sub-b8 | 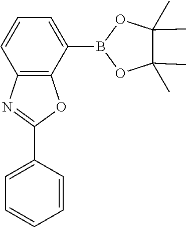 [SMILES: CC1(C)OB(c2cccc3nc(-c4ccccc4)oc23)OC1(C)C] | 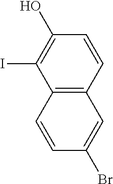 [SMILES: Oc1ccc2cc(Br)ccc2c1I] CAS: 102153-45-7 | 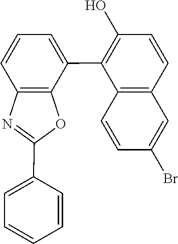 [SMILES: Oc1ccc2cc(Br)ccc2c1-c1cccc2nc(-c3ccccc3)oc12] | 53 |
| Sub-b9 | 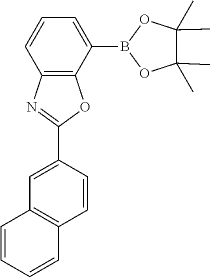 [SMILES: CC1(C)OB(c2cccc3nc(-c4ccc5ccccc5c4)oc23)OC1(C)C] | 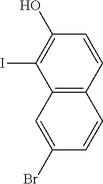 [SMILES: Oc1ccc2ccc(Br)cc2c1I] CAS: 2361499-19-4 | 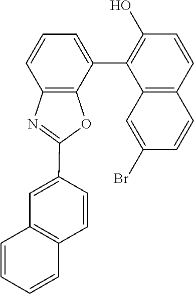 [SMILES: Oc1ccc2ccc(Br)cc2c1-c1cccc2nc(-c3ccc4ccccc4c3)oc12] | 45 |
| Sub-b10 | 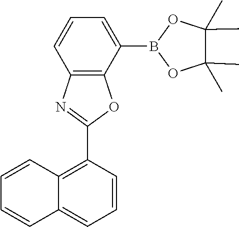 [SMILES: CC1(C)OB(c2cccc3nc(-c4cccc5ccccc45)oc23)OC1(C)C] | 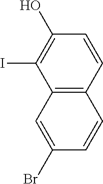 [SMILES: Oc1ccc2ccc(Br)cc2c1I] CAS: 2361499-19-4 | 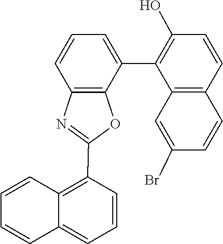 [SMILES: Oc1ccc2ccc(Br)cc2c1-c1cccc2nc(-c3cccc4ccccc34)oc12] | 42 |
| Sub-b11 | 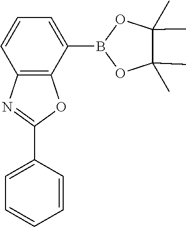 [SMILES: CC1(C)OB(c2cccc3nc(-c4ccccc4)oc23)OC1(C)C] | 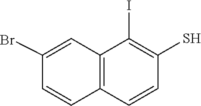 [SMILES: Sc1ccc2ccc(Br)cc2c1I] | 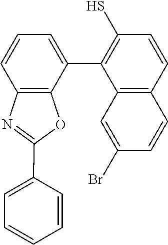 [SMILES: Sc1ccc2ccc(Br)cc2c1-c1cccc2nc(-c3ccccc3)oc12] | 42 |

### Synthesis of Sub-c1:

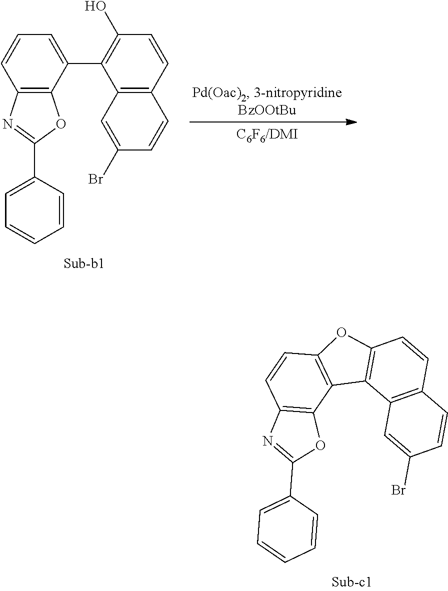 [SMILES: Oc1ccc2ccc(Br)cc2c1-c1cccc2nc(-c3ccccc3)oc12 ; Brc1ccc2ccc3oc4ccc5nc(-c6ccccc6)oc5c4c3c2c1]

Under nitrogen atmosphere, Sub-b1 (20.81 g, 50 mmol), tert-butyl peroxybenzoate (BzOOt-Bu, 19.42 g, 100 mmol), palladium acetate (1.12 g, 5 mmol), 3-nitropyridine (0.62 g, 5 mmol), hexafluorobenzene (C₆F₆, 210 mL), and N,N′-dimethyl imidazolidinone (DMI, 140 mL) were sequentially added into a 500 mL of three-necked flask, and the system was stirred and heated to 90° C. and reacted for 4 hours. The system was cooled to room temperature and then extracted with ethyl acetate (100 mL×3 times), organic phases were dried with anhydrous magnesium sulfate, filtrated, and the filtrate was distilled in vacuum to remove solvent, and a crude product was obtained. The crude product was purified by silica gel column chromatography with n-heptane/dichloromethane as a mobile phase to obtain Sub-c1 as a white solid (10.77 g, yield 52%).

With reference to Sub-c1, Sub-b1 was replaced by Reactant D shown in Table 3 to synthesize Sub-c2 to Sub-c10.

TABLE 3
Synthesis of Sub-c2 to Sub-c10

| Sub-c No. | Reactant D | Structure of Sub-c | Yield (%) |
|---|---|---|---|
| Sub-c2 | 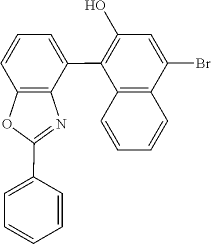 [SMILES: Oc1cc(Br)c2ccccc2c1-c1cccc2oc(-c3ccccc3)nc12] | 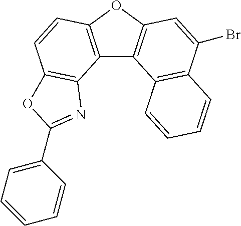 [SMILES: Brc1cc2oc3ccc4oc(-c5ccccc5)nc4c3c2c2ccccc12] | 58 |
| Sub-c3 | 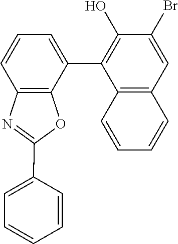 [SMILES: Oc1c(Br)cc2ccccc2c1-c1cccc2nc(-c3ccccc3)oc12] | 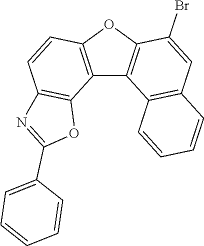 [SMILES: Brc1cc2ccccc2c2c1oc1ccc3nc(-c4ccccc4)oc3c12] | 61 |
| Sub-c4 | 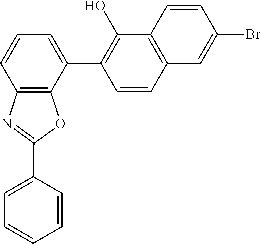 [SMILES: Oc1c(-c2cccc3nc(-c4ccccc4)oc23)ccc2cc(Br)ccc12] | 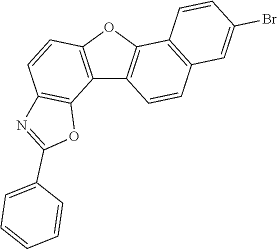 [SMILES: Brc1ccc2c(ccc3c2oc2ccc4nc(-c5ccccc5)oc4c23)c1] | 53 |
| Sub-c5 | 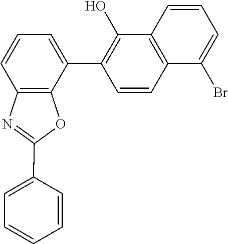 [SMILES: Oc1c(-c2cccc3nc(-c4ccccc4)oc23)ccc2c(Br)cccc12] | 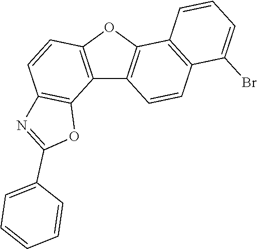 [SMILES: Brc1cccc2c1ccc1c2oc2ccc3nc(-c4ccccc4)oc3c21] | 64 |
| Sub-c6 | 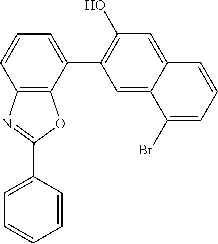 [SMILES: Oc1cc2cccc(Br)c2cc1-c1cccc2nc(-c3ccccc3)oc12] | 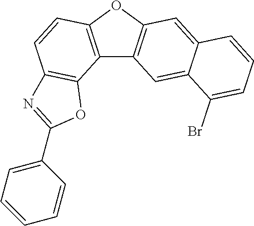 [SMILES: Brc1cccc2cc3oc4ccc5nc(-c6ccccc6)oc5c4c3cc12] | 54 |
| Sub-c7 | 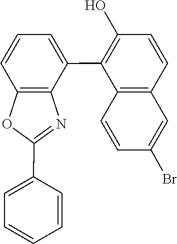 [SMILES: Oc1ccc2cc(Br)ccc2c1-c1cccc2oc(-c3ccccc3)nc12] | 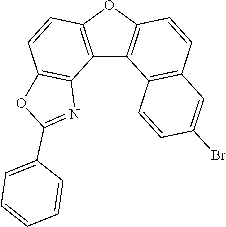 [SMILES: Brc1ccc2c(ccc3oc4ccc5oc(-c6ccccc6)nc5c4c32)c1] | 53 |
| Sub-c8 | 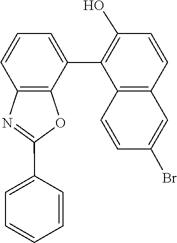 [SMILES: Oc1ccc2cc(Br)ccc2c1-c1cccc2nc(-c3ccccc3)oc12] | 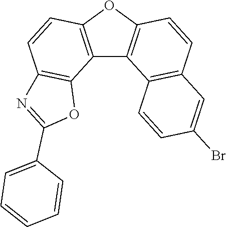 [SMILES: Brc1ccc2c(ccc3oc4ccc5nc(-c6ccccc6)oc5c4c32)c1] | 57 |
| Sub-c9 | 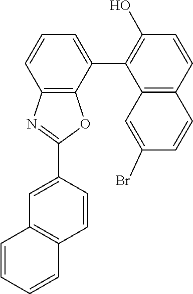 [SMILES: Oc1ccc2ccc(Br)cc2c1-c1cccc2nc(-c3ccc4ccccc4c3)oc12] | 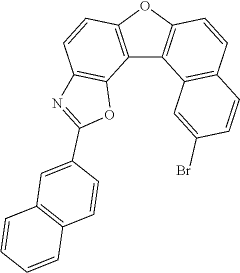 [SMILES: Brc1ccc2ccc3oc4ccc5nc(-c6ccc7ccccc7c6)oc5c4c3c2c1] | 56 |
| Sub-c10 | 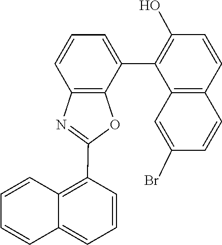 [SMILES: Oc1ccc2ccc(Br)cc2c1-c1cccc2nc(-c3cccc4ccccc34)oc12] | 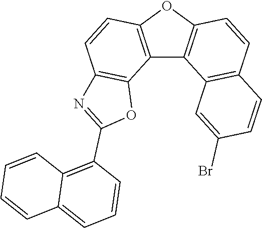 [SMILES: Brc1ccc2ccc3oc4ccc5nc(-c6cccc7ccccc67)oc5c4c3c2c1] | 51 |

### Synthesis of Sub-c11:

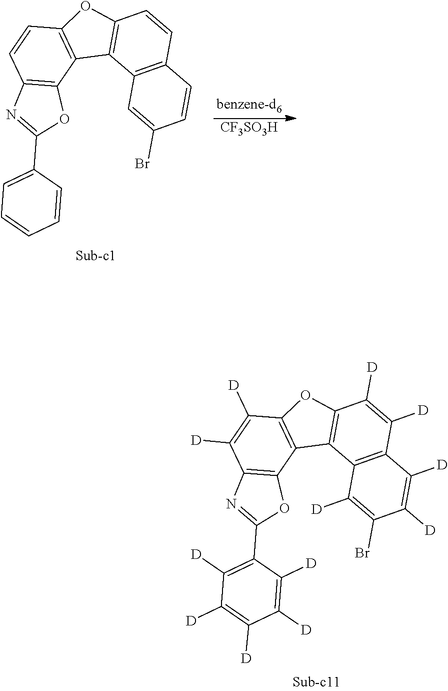

Under nitrogen atmosphere, Sub-c1 (10.36 g, 25 mmol) and 200 mL of deuterobenzene-D6 were added to a 100 mL of three-necked flask and heated to 60° C., then trifluoromethanesulfonic acid (22.51 g, 150 mmol) was added, the heating was continued until boiling, and the system was stirred and reacted for 24 hours. After the reaction system was cooled to room temperature, 50 mL of heavy water was added, the system was stirred for 10 minutes, and then a saturated K₃PO₄ aqueous solution was added to neutralize the reaction solution. Organic layers were extracted with dichloromethane (50 mL×3 times), organic phases were combined and dried with anhydrous sodium sulfate, filtrated, and the filtrate was distilled in vacuum to remove solvent, and a crude product was obtained. The crude product was purified by silica gel column chromatography with n-heptane/dichloromethane as a mobile phase to obtain Sub-c11 as a white solid (6.82 g, yield 64%).

### Synthesis of Sub-c12:

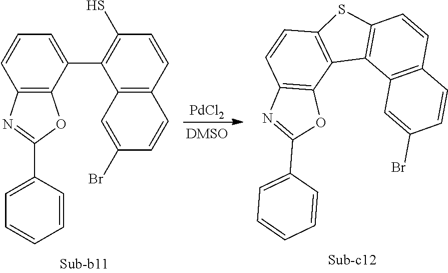

Under nitrogen atmosphere, Sub-b11 (10.80 g, 25 mmol), palladium dichloride (0.22 g, 1.25 mmol) and DMSO (120 mL) were added to a 250 mL of three-necked flask, heated to 140° C., and stirred for 12 hours. After the reaction system was cooled to room temperature, organic layers were extracted with dichloromethane (50 mL×3 times), organic phases were combined and dried with anhydrous sodium sulfate, filtrated, and the filtrate was distilled in vacuum to remove solvent, and a crude product was obtained. The crude product was purified by silica gel column chromatography with n-heptane/dichloromethane as a mobile phase to obtain Sub-c12 as a white solid (7.85 g, yield 73%).

### Synthesis of Sub-d1:

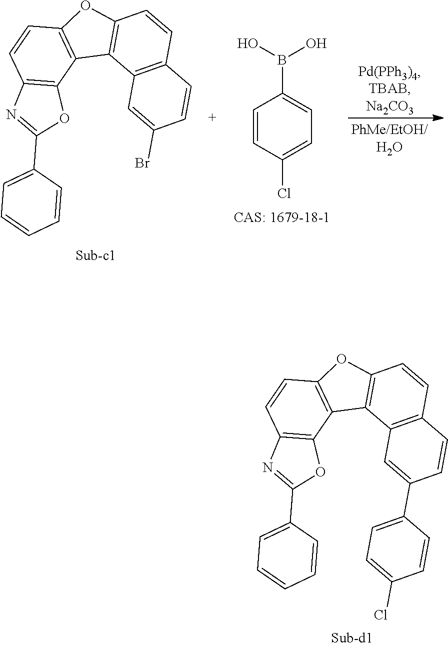 [SMILES: Brc1ccc2ccc3oc4ccc5nc(-c6ccccc6)oc5c4c3c2c1 ; OB(O)c1ccc(Cl)cc1 ; Clc1ccc(-c2ccc3ccc4oc5ccc6nc(-c7ccccc7)oc6c5c4c3c2)cc1]

Under nitrogen atmosphere, Sub-c1 (13.36 g, 50 mmol), 4-chlorophenylboronic acid (8.60 g, 55 mmol), tetra (triphenylphosphine) palladium (Pd(PPh₃)₄, 0.58 g, 0 5 mmol), anhydrous sodium carbonate (10.60 g, 100 mmol), toluene (140 mL), anhydrous ethanol (35 mL), and deionized water (35 mL) were sequentially added into a 500 mL of three-necked flask, stirring and heating until reflux were performed, and the system reacted for 16 hours. The system was cooled to room temperature and then extracted with dichloromethane (100 mL×3 times), organic phases were combined and dried with anhydrous magnesium sulfate, filtrated, and the filtrate was distilled in vacuum to remove solvent, and a crude product was obtained. The crude product was purified by silica gel column chromatography with n-heptane as a mobile phase to obtain Sub-d1 as a white solid (13.82 g, yield 62%).

With reference to the synthesis of Sub-d1, Sub-c was replaced by Reactant E as shown in Table 4, and 4-chlorophenylboronic acid was replaced by Reactant F to synthesize Sub-d2 to Sub-d8.

TABLE 4
Synthesis of Sub-d2 to Sub-d8

| Sub-d | | | | |
|---|---|---|---|---|
| No. | Reactant E | Reactant F | Structure of Sub-d | Yield (%) |
| Sub-d2 | 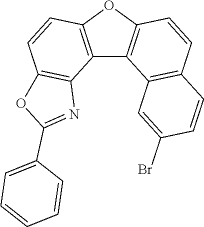 [SMILES: Brc1ccc2ccc3oc4ccc5oc(-c6ccccc6)nc5c4c3c2c1] | 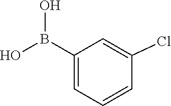 CAS: 63503-60-6 | 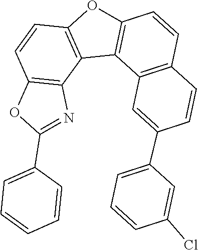 [SMILES: Clc1cccc(-c2ccc3ccc4oc5ccc6oc(-c7ccccc7)nc6c5c4c3c2)c1] | 65 |
| Sub-d3 | 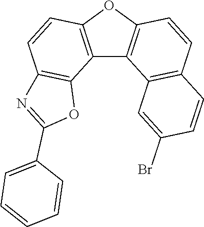 [SMILES: Brc1ccc2ccc3oc4ccc5nc(-c6ccccc6)oc5c4c3c2c1] | 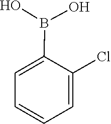 CAS: 3900-89-8 | 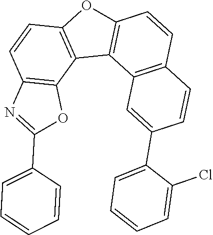 [SMILES: Clc1ccccc1-c1ccc2ccc3oc4ccc5nc(-c6ccccc6)oc5c4c3c2c1] | 58 |
| Sub-d4 | 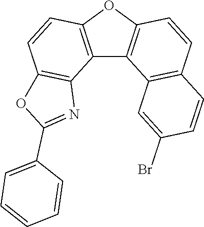 [SMILES: Brc1ccc2ccc3oc4ccc5oc(-c6ccccc6)nc5c4c3c2c1] | 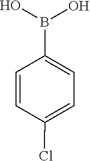 CAS: 1679-18-1 | 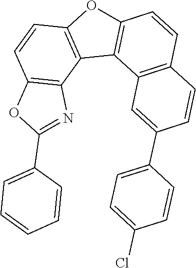 [SMILES: Clc1ccc(-c2ccc3ccc4oc5ccc6oc(-c7ccccc7)nc6c5c4c3c2)cc1] | 65 |
| Sub-d5 | 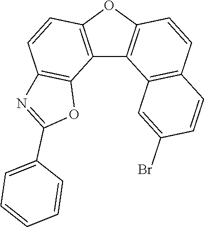 [SMILES: Brc1ccc2ccc3oc4ccc5nc(-c6ccccc6)oc5c4c3c2c1] | 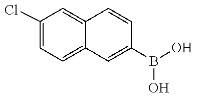 [SMILES: OB(O)c1ccc2cc(Cl)ccc2c1] CAS: 870822-86-9 | 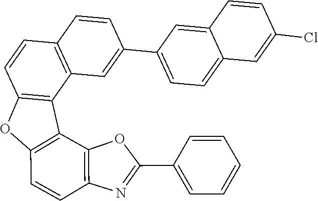 [SMILES: Clc1ccc2cc(-c3ccc4ccc5oc6ccc7nc(-c8ccccc8)oc7c6c5c4c3)ccc2c1] | 56 |
| Sub-d6 | 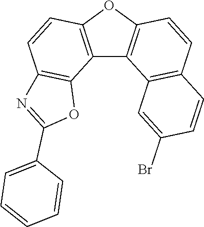 [SMILES: Brc1ccc2ccc3oc4ccc5nc(-c6ccccc6)oc5c4c3c2c1] | 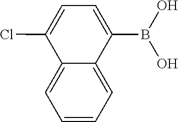 [SMILES: OB(O)c1ccc(Cl)c2ccccc12] CAS: 147102-97-4 | 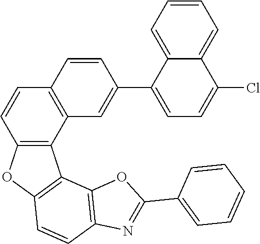 [SMILES: Clc1ccc(-c2ccc3ccc4oc5ccc6nc(-c7ccccc7)oc6c5c4c3c2)c2ccccc12] | 59 |
| Sub-d7 | 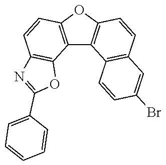 [SMILES: Brc1ccc2c(ccc3oc4ccc5nc(-c6ccccc6)oc5c4c32)c1] | 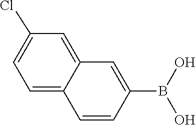 [SMILES: OB(O)c1ccc2ccc(Cl)cc2c1] CAS: 2118958-50-0 | 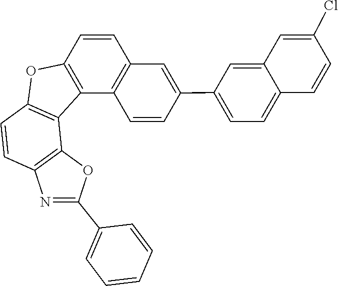 [SMILES: Clc1ccc2ccc(-c3ccc4c(ccc5oc6ccc7nc(-c8ccccc8)oc7c6c54)c3)cc2c1] | 53 |
| Sub-d8 | 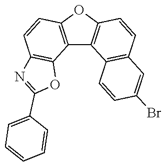 [SMILES: Brc1ccc2c(ccc3oc4ccc5nc(-c6ccccc6)oc5c4c32)c1] | 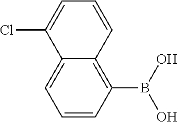 [SMILES: OB(O)c1cccc2c(Cl)cccc12] CAS: 2142655-40-9 | 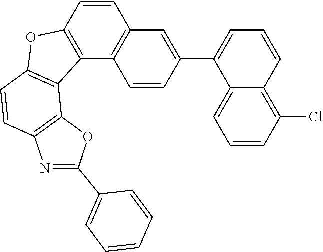 [SMILES: Clc1cccc2c(-c3ccc4c(ccc5oc6ccc7nc(-c8ccccc8)oc7c6c54)c3)cccc12] | 52 |

### Synthesis of Compound 3:

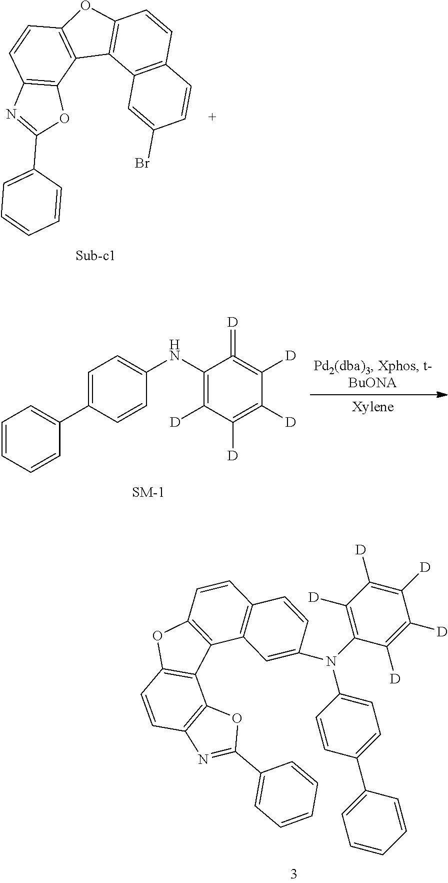

Under nitrogen atmosphere, Sub-c1 (10.35 g, 25 mmol), SM-1 (CAS: 1322090-81-2, 6.88 g, 27.5 mmol), tri(dibenzalacetone)dipalladium (0.46 g, 0.5 mmol), 2-dicyclohexylphosphine-2′,4′,6′-triisopropylbiphenyl (0.48 g, 1 mmol), sodium tert-butoxide (4.80 g, 50 mmol) and xylene (100 mL) were sequentially added into a 250 mL of three-necked flask, heated until reflux, and stirred for reaction overnight; and the system was cooled to room temperature and then extracted with dichloromethane (100 mL×3 times), organic phases were combined and dried with anhydrous magnesium sulfate, filtrated, and the filtrate was distilled in vacuum to remove solvent and obtain a crude product. The crude product was purified by silica gel column chromatography with n-heptane as a mobile phase to obtain Compound 3 as a white solid (7.73 g, yield 53%, m/z=584.2 [M+H]⁺).

With reference to the synthesis of Compound 3, Sub-c1 was replaced by Reactant G shown in Table 5, and SM-1 was replaced by Reactant H to synthesize Compounds in Table 5.

TABLE 5
Synthesis of Compounds

| | | Structure and No. of | m/z | |
|---|---|---|---|---|
| Reactant G | Reactant H | compound | ([M + H]⁺) | Yield (%) |
| 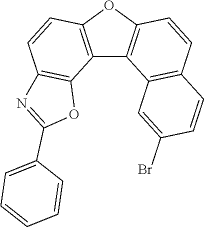 [SMILES: Brc1ccc2ccc3oc4ccc5nc(-c6ccccc6)oc5c4c3c2c1] Sub-c1 | 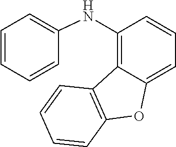 [SMILES: c1ccc(Nc2cccc3oc4ccccc4c23)cc1] CAS: 1325195-27-4 | 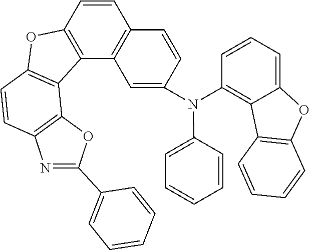 [SMILES: c1ccc(-c2nc3ccc4oc5ccc6ccc(N(c7ccccc7)c7cccc8oc9ccccc9c78)cc6c5c4c3o2)cc1] | 593.2 | 60 |
| | | 6 | | |
| 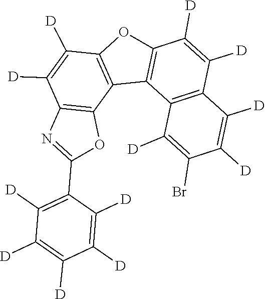 [SMILES: [2H]c1c([2H])c([2H])c(-c2nc3c([2H])c([2H])c4oc5c([2H])c([2H])c6c([2H])c([2H])c(Br)c([2H])c6c5c4c3o2)c([2H])c1[2H]] Sub-c11 | 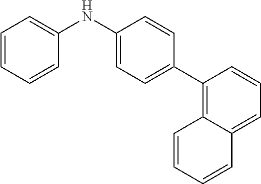 CAS: 897671-78-2 | 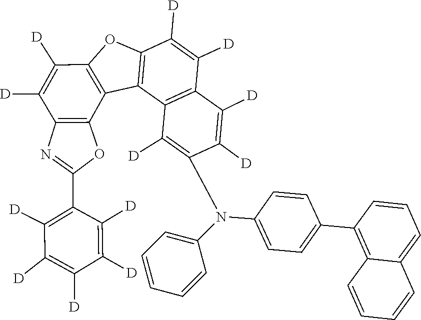 [SMILES: [2H]c1c([2H])c([2H])c(-c2nc3c([2H])c([2H])c4oc5c([2H])c([2H])c6c([2H])c([2H])c(N(c7ccccc7)c7ccc(-c8cccc9ccccc89)cc7)c([2H])c6c5c4c3o2)c([2H])c1[2H]] | 641.3 | 53 |
| | | 11 | | |
| 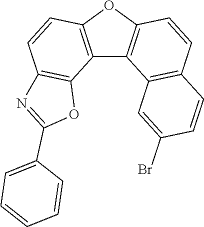 [SMILES: Brc1ccc2ccc3oc4ccc5nc(-c6ccccc6)oc5c4c3c2c1] Sub-c1 | 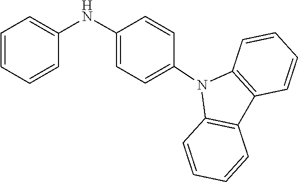 [SMILES: c1ccc(Nc2ccc(-n3c4ccccc4c4ccccc43)cc2)cc1] CAS: 858641-06-2 | 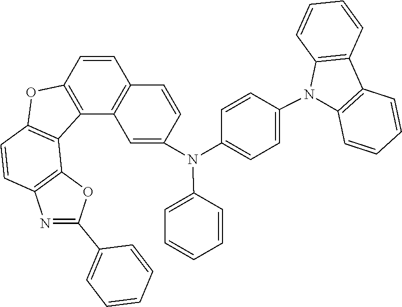 [SMILES: c1ccc(-c2nc3ccc4oc5ccc6ccc(N(c7ccccc7)c7ccc(-n8c9ccccc9c9ccccc98)cc7)cc6c5c4c3o2)cc1] | 668.2 | 57 |
| | | 14 | | |
| 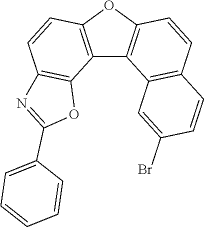 [SMILES: Brc1ccc2ccc3oc4ccc5nc(-c6ccccc6)oc5c4c3c2c1] Sub-c1 | 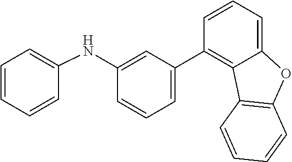 [SMILES: c1ccc(Nc2cccc(-c3cccc4oc5ccccc5c34)c2)cc1] CAS: 2271355-33-8 | 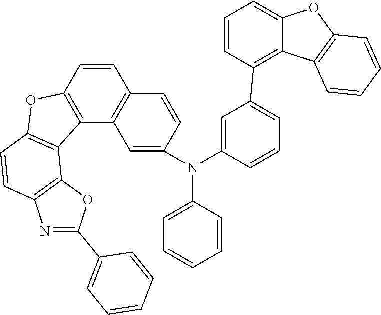 [SMILES: c1ccc(-c2nc3ccc4oc5ccc6ccc(N(c7ccccc7)c7cccc(-c8cccc9oc%10ccccc%10c89)c7)cc6c5c4c3o2)cc1] | 669.2 | 55 |
| | | 19 | | |
| 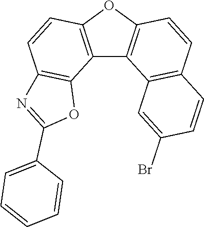 [SMILES: Brc1ccc2ccc3oc4ccc5nc(-c6ccccc6)oc5c4c3c2c1] Sub-c1 | 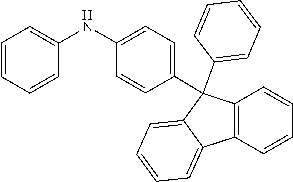 [SMILES: c1ccc(Nc2ccc(C3(c4ccccc4)c4ccccc4-c4ccccc43)cc2)cc1] CAS: 851343-53-8 | 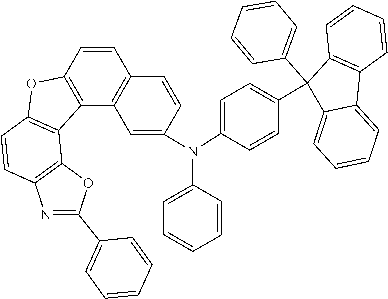 [SMILES: c1ccc(-c2nc3ccc4oc5ccc6ccc(N(c7ccccc7)c7ccc(C8(c9ccccc9)c9ccccc9-c9ccccc98)cc7)cc6c5c4c3o2)cc1] | 743.3 | 51 |
| | | 22 | | |
| 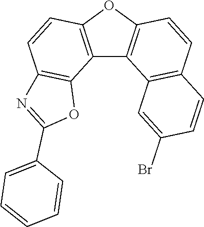 [SMILES: Brc1ccc2ccc3oc4ccc5nc(-c6ccccc6)oc5c4c3c2c1] Sub-c1 | 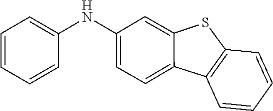 [SMILES: c1ccc(Nc2ccc3c(c2)sc2ccccc23)cc1] CAS: 1427556-53-3 | 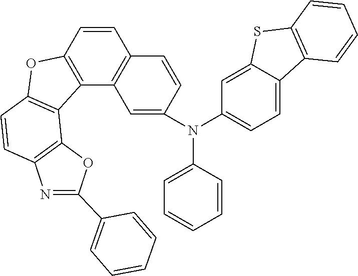 [SMILES: c1ccc(-c2nc3ccc4oc5ccc6ccc(N(c7ccccc7)c7ccc8c(c7)sc7ccccc78)cc6c5c4c3o2)cc1] | 609.2 | 54 |
| | | 29 | | |
| 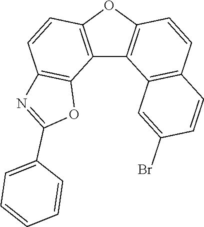 [SMILES: Brc1ccc2ccc3oc4ccc5nc(-c6ccccc6)oc5c4c3c2c1] Sub-c1 | 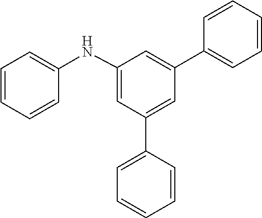 [SMILES: c1ccc(Nc2cc(-c3ccccc3)cc(-c3ccccc3)c2)cc1] CAS: 1065663-41-3 | 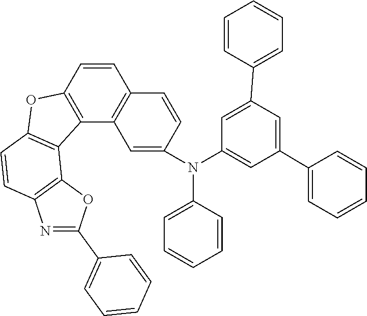 [SMILES: c1ccc(-c2cc(-c3ccccc3)cc(N(c3ccccc3)c3ccc4ccc5oc6ccc7nc(-c8ccccc8)oc7c6c5c4c3)c2)cc1] | 655.2 | 52 |
| | | 39 | | |
| 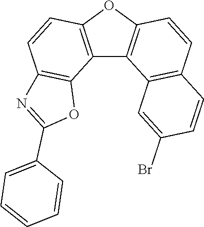 [SMILES: Brc1ccc2ccc3oc4ccc5nc(-c6ccccc6)oc5c4c3c2c1] Sub-c1 | 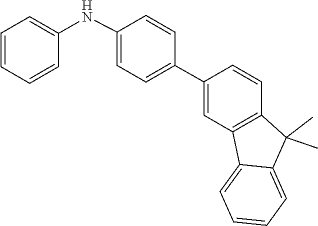 [SMILES: CC1(C)c2ccccc2-c2cc(-c3ccc(Nc4ccccc4)cc3)ccc21] CAS: 1644466-64-7 | 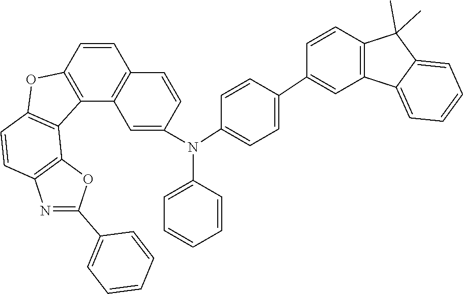 [SMILES: CC1(C)c2ccccc2-c2cc(-c3ccc(N(c4ccccc4)c4ccc5ccc6oc7ccc8nc(-c9ccccc9)oc8c7c6c5c4)cc3)ccc21] | 695.3 | 58 |
| | | 48 | | |
| 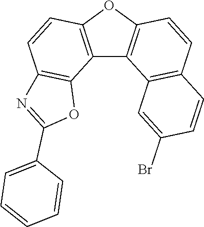 [SMILES: Brc1ccc2ccc3oc4ccc5nc(-c6ccccc6)oc5c4c3c2c1] Sub-c1 | 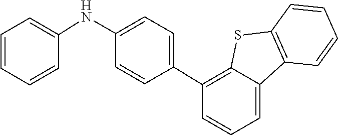 [SMILES: c1ccc(Nc2ccc(-c3cccc4c3sc3ccccc34)cc2)cc1] CAS: 1357572-30-5 | 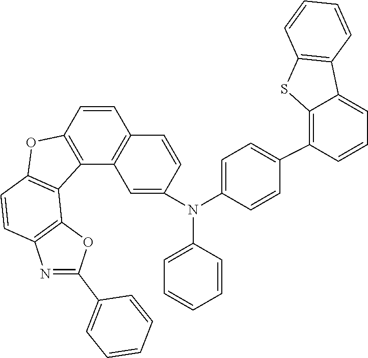 [SMILES: c1ccc(-c2nc3ccc4oc5ccc6ccc(N(c7ccccc7)c7ccc(-c8cccc9c8sc8ccccc89)cc7)cc6c5c4c3o2)cc1] | 685.2 | 57 |
| | | 52 | | |
| 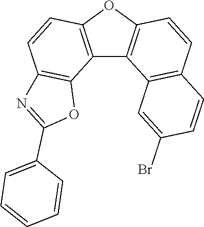 [SMILES: Brc1ccc2ccc3oc4ccc5nc(-c6ccccc6)oc5c4c3c2c1] Sub-c1 | 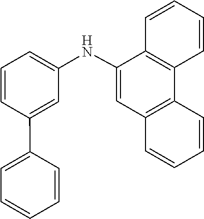 [SMILES: c1ccc(-c2cccc(Nc3cc4ccccc4c4ccccc34)c2)cc1] CAS: 1372778-28-3 | 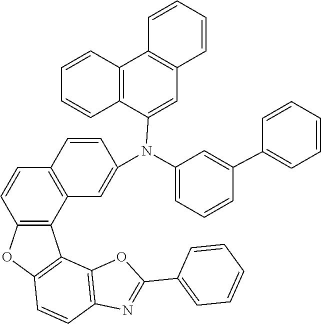 [SMILES: c1ccc(-c2cccc(N(c3ccc4ccc5oc6ccc7nc(-c8ccccc8)oc7c6c5c4c3)c3cc4ccccc4c4ccccc34)c2)cc1] | 679.2 | 50 |
| | | 76 | | |
| 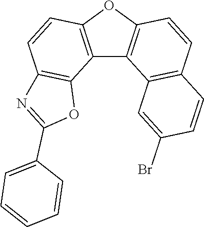 [SMILES: Brc1ccc2ccc3oc4ccc5nc(-c6ccccc6)oc5c4c3c2c1] Sub-c1 | 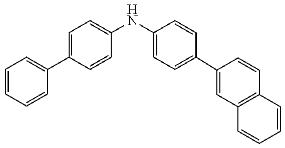 [SMILES: c1ccc(-c2ccc(Nc3ccc(-c4ccc5ccccc5c4)cc3)cc2)cc1] CAS: 897921-60-7 | 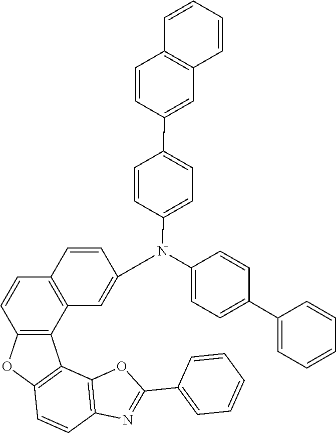 [SMILES: c1ccc(-c2ccc(N(c3ccc(-c4ccc5ccccc5c4)cc3)c3ccc4ccc5oc6ccc7nc(-c8ccccc8)oc7c6c5c4c3)cc2)cc1] | 705.2 | 56 |
| | | 79 | | |
| 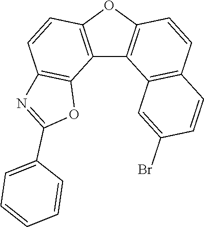 [SMILES: Brc1ccc2ccc3oc4ccc5nc(-c6ccccc6)oc5c4c3c2c1] Sub-c1 | 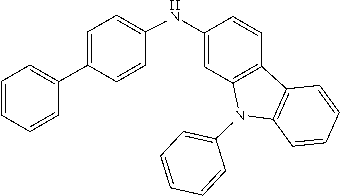 [SMILES: c1ccc(-c2ccc(Nc3ccc4c5ccccc5n(-c5ccccc5)c4c3)cc2)cc1] CAS: 1427316-58-2 | 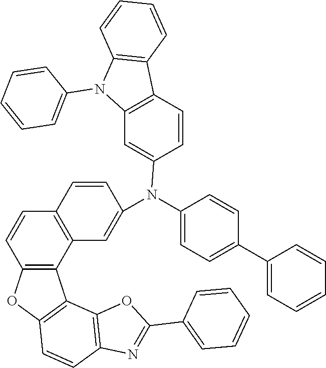 [SMILES: c1ccc(-c2ccc(N(c3ccc4ccc5oc6ccc7nc(-c8ccccc8)oc7c6c5c4c3)c3ccc4c5ccccc5n(-c5ccccc5)c4c3)cc2)cc1] | 744.3 | 59 |
| | | 102 | | |
| 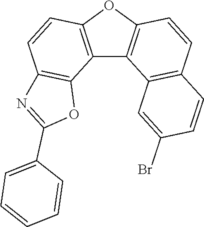 [SMILES: Brc1ccc2ccc3oc4ccc5nc(-c6ccccc6)oc5c4c3c2c1] Sub-c1 | 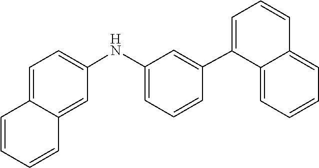 [SMILES: c1cc(Nc2ccc3ccccc3c2)cc(-c2cccc3ccccc23)c1] CAS: 2634130-32-6 | 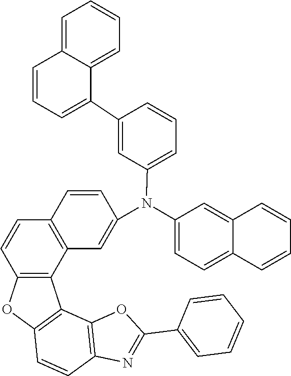 [SMILES: c1ccc(-c2nc3ccc4oc5ccc6ccc(N(c7cccc(-c8cccc9ccccc89)c7)c7ccc8ccccc8c7)cc6c5c4c3o2)cc1] | 679.2 | 51 |
| | | 130 | | |
| 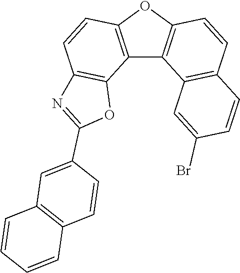 [SMILES: Brc1ccc2ccc3oc4ccc5nc(-c6ccc7ccccc7c6)oc5c4c3c2c1] Sub-c9 | 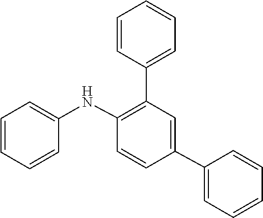 [SMILES: c1ccc(Nc2ccc(-c3ccccc3)cc2-c2ccccc2)cc1] CAS: 1417334-01-0 | 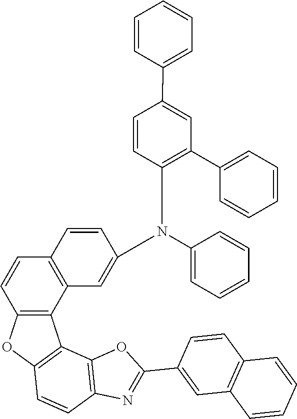 [SMILES: c1ccc(-c2ccc(N(c3ccccc3)c3ccc4ccc5oc6ccc7nc(-c8ccc9ccccc9c8)oc7c6c5c4c3)c(-c3ccccc3)c2)cc1] | 705.2 | 52 |
| | | 178 | | |
| 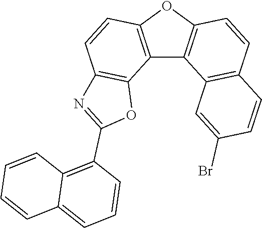 [SMILES: Brc1ccc2ccc3oc4ccc5nc(-c6cccc7ccccc67)oc5c4c3c2c1] Sub-c10 | 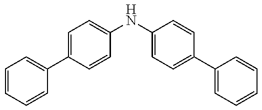 [SMILES: c1ccc(-c2ccc(Nc3ccc(-c4ccccc4)cc3)cc2)cc1] CAS: 102113-98-4 | 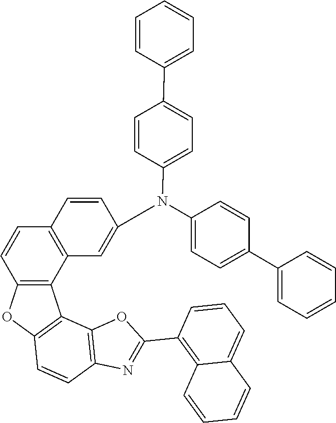 [SMILES: c1ccc(-c2ccc(N(c3ccc(-c4ccccc4)cc3)c3ccc4ccc5oc6ccc7nc(-c8cccc9ccccc89)oc7c6c5c4c3)cc2)cc1] | 705.2 | 60 |
| | | 181 | | |
| 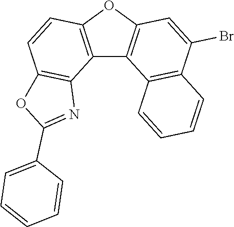 [SMILES: Brc1cc2oc3ccc4oc(-c5ccccc5)nc4c3c2c2ccccc12] Sub-c2 | 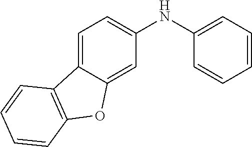 [SMILES: c1ccc(Nc2ccc3c(c2)oc2ccccc23)cc1] CAS: 406488-21-9 | 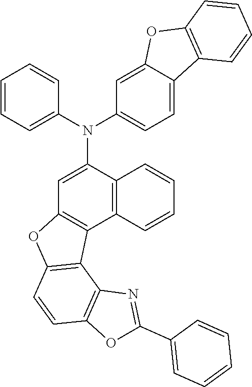 [SMILES: c1ccc(-c2nc3c(ccc4oc5cc(N(c6ccccc6)c6ccc7c(c6)oc6ccccc67)c6ccccc6c5c43)o2)cc1] | 593.2 | 57 |
| | | 206 | | |
| 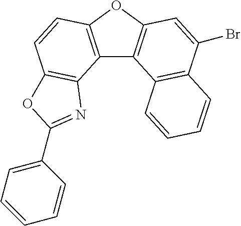 [SMILES: Brc1cc2oc3ccc4oc(-c5ccccc5)nc4c3c2c2ccccc12] Sub-c2 | 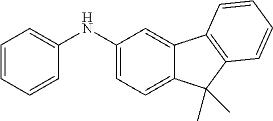 [SMILES: CC1(C)c2ccccc2-c2cc(Nc3ccccc3)ccc21] CAS: 850181-65-6 | 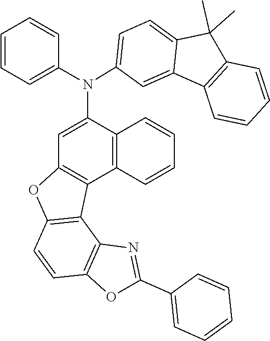 [SMILES: CC1(C)c2ccccc2-c2cc(N(c3ccccc3)c3cc4oc5ccc6oc(-c7ccccc7)nc6c5c4c4ccccc34)ccc21] | 619.2 | 58 |
| | | 210 | | |
| 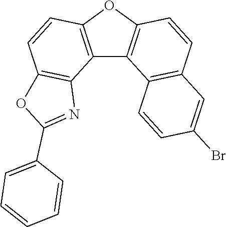 [SMILES: Brc1ccc2c(ccc3oc4ccc5oc(-c6ccccc6)nc5c4c32)c1] Sub-c7 | 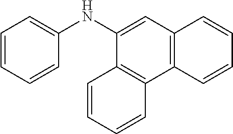 CAS: 3920-79-4 | 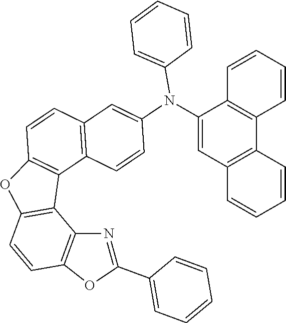 [SMILES: c1ccc(-c2nc3c(ccc4oc5ccc6cc(N(c7ccccc7)c7cc8ccccc8c8ccccc78)ccc6c5c43)o2)cc1] | 603.2 | 52 |
| | | 212 | | |
| 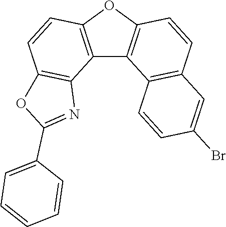 [SMILES: Brc1ccc2c(ccc3oc4ccc5oc(-c6ccccc6)nc5c4c32)c1] Sub-c7 | 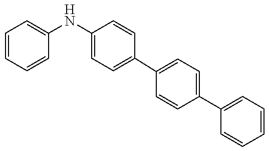 [SMILES: c1ccc(Nc2ccc(-c3ccc(-c4ccccc4)cc3)cc2)cc1] CAS: 897671-81-7 | 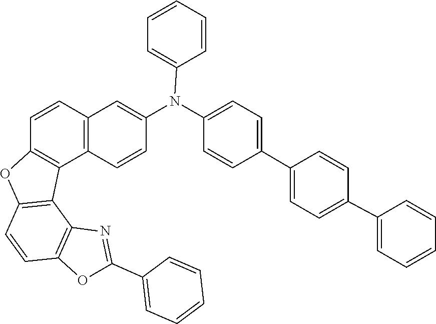 [SMILES: c1ccc(-c2ccc(-c3ccc(N(c4ccccc4)c4ccc5c(ccc6oc7ccc8oc(-c9ccccc9)nc8c7c65)c4)cc3)cc2)cc1] | 655.2 | 56 |
| | | 216 | | |
| 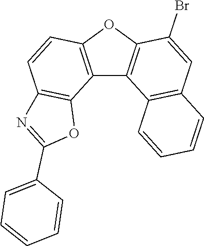 [SMILES: Brc1cc2ccccc2c2c1oc1ccc3nc(-c4ccccc4)oc3c12] Sub-c3 | 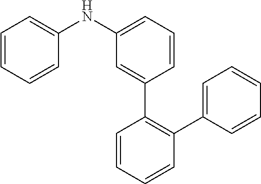 CAS: 1222634-04-9 | 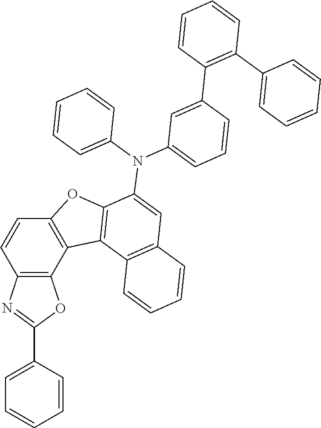 [SMILES: c1ccc(-c2nc3ccc4oc5c(N(c6ccccc6)c6cccc(-c7ccccc7-c7ccccc7)c6)cc6ccccc6c5c4c3o2)cc1] | 655.2 | 54 |
| | | 229 | | |
| 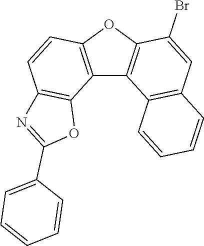 [SMILES: Brc1cc2ccccc2c2c1oc1ccc3nc(-c4ccccc4)oc3c12] Sub-c3 | 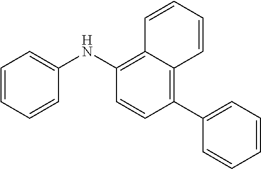 [SMILES: c1ccc(Nc2ccc(-c3ccccc3)c3ccccc23)cc1] CAS: 897671-76-0 | 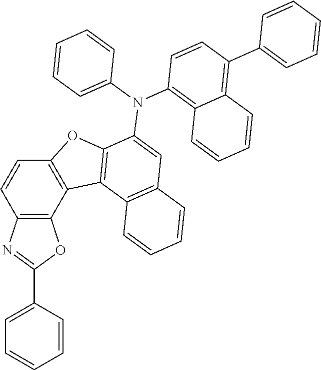 [SMILES: c1ccc(-c2nc3ccc4oc5c(N(c6ccccc6)c6ccc(-c7ccccc7)c7ccccc67)cc6ccccc6c5c4c3o2)cc1] | 629.2 | 51 |
| | | 233 | | |
| 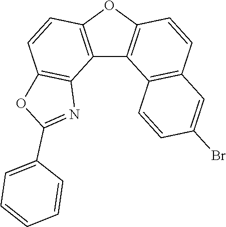 [SMILES: Brc1ccc2c(ccc3oc4ccc5oc(-c6ccccc6)nc5c4c32)c1] Sub-c7 | 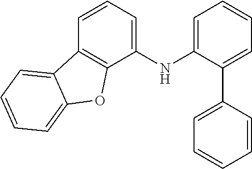 [SMILES: c1ccc(-c2ccccc2Nc2cccc3c2oc2ccccc23)cc1] CAS: 1252914-36-5 | 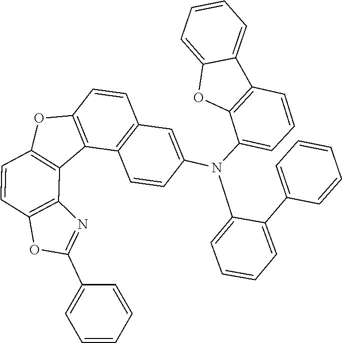 [SMILES: c1ccc(-c2nc3c(ccc4oc5ccc6cc(N(c7ccccc7-c7ccccc7)c7cccc8c7oc7ccccc78)ccc6c5c43)o2)cc1] | 669.2 | 52 |
| | | 252 | | |
| 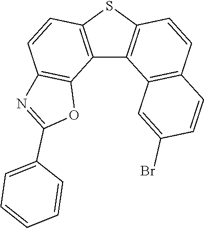 [SMILES: Brc1ccc2ccc3sc4ccc5nc(-c6ccccc6)oc5c4c3c2c1] Sub-c12 | 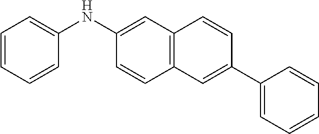 [SMILES: c1ccc(Nc2ccc3cc(-c4ccccc4)ccc3c2)cc1] CAS: 1300028-96-9 | 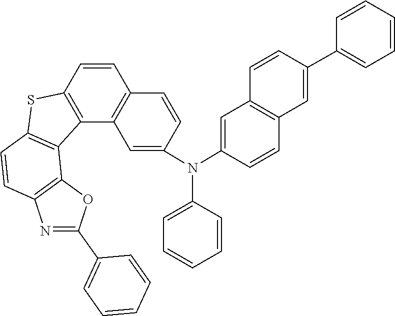 [SMILES: c1ccc(-c2ccc3cc(N(c4ccccc4)c4ccc5ccc6sc7ccc8nc(-c9ccccc9)oc8c7c6c5c4)ccc3c2)cc1] | 645.2 | 53 |
| | | 268 | | |
| 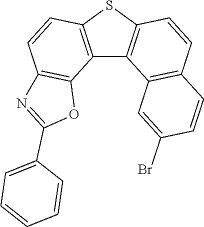 [SMILES: Brc1ccc2ccc3sc4ccc5nc(-c6ccccc6)oc5c4c3c2c1] Sub-c12 | 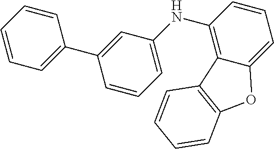 [SMILES: c1ccc(-c2cccc(Nc3cccc4oc5ccccc5c34)c2)cc1] CAS: 2230816-66-5 | 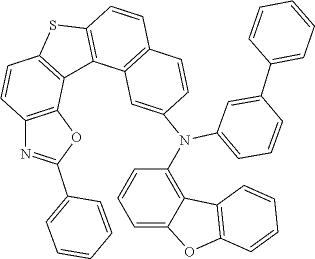 [SMILES: c1ccc(-c2cccc(N(c3ccc4ccc5sc6ccc7nc(-c8ccccc8)oc7c6c5c4c3)c3cccc4oc5ccccc5c34)c2)cc1] | 685.2 | 52 |
| | | 293 | | |
| 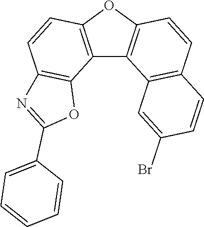 [SMILES: Brc1ccc2ccc3oc4ccc5nc(-c6ccccc6)oc5c4c3c2c1] Sub-c1 | 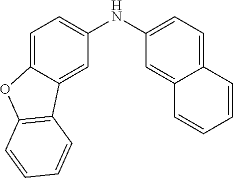 [SMILES: c1ccc2cc(Nc3ccc4oc5ccccc5c4c3)ccc2c1] CAS: 1621976-98-4 | 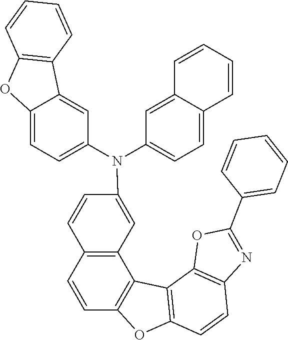 [SMILES: c1ccc(-c2nc3ccc4oc5ccc6ccc(N(c7ccc8ccccc8c7)c7ccc8oc9ccccc9c8c7)cc6c5c4c3o2)cc1] | 643.2 | 52 |
| | | 327 | | |
| 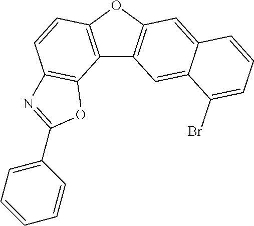 [SMILES: Brc1cccc2cc3oc4ccc5nc(-c6ccccc6)oc5c4c3cc12] Sub-c6 | 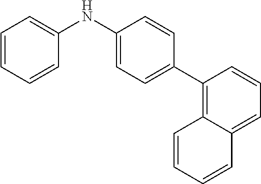 CAS: 897671-78-2 | 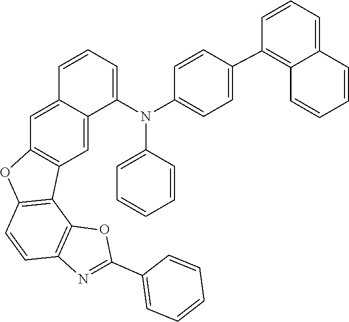 [SMILES: c1ccc(-c2nc3ccc4oc5cc6cccc(N(c7ccccc7)c7ccc(-c8cccc9ccccc89)cc7)c6cc5c4c3o2)cc1] | 629.2 | 55 |
| | | 346 | | |
| 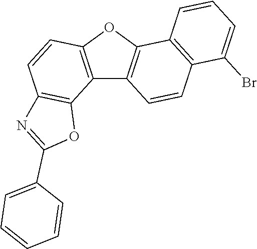 [SMILES: Brc1cccc2c1ccc1c2oc2ccc3nc(-c4ccccc4)oc3c21] Sub-c5 | 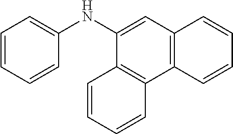 CAS: 3920-79-4 | 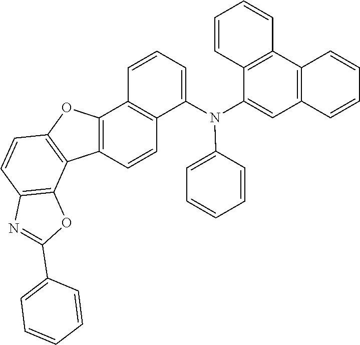 [SMILES: c1ccc(-c2nc3ccc4oc5c6cccc(N(c7ccccc7)c7cc8ccccc8c8ccccc78)c6ccc5c4c3o2)cc1] | 603.2 | 51 |
| | | 348 | | |
| 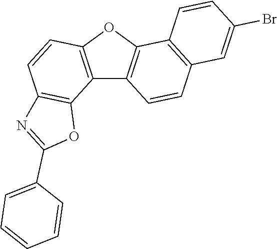 [SMILES: Brc1ccc2c(ccc3c2oc2ccc4nc(-c5ccccc5)oc4c23)c1] Sub-c4 | 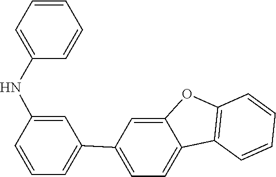 [SMILES: c1ccc(Nc2cccc(-c3ccc4c(c3)oc3ccccc34)c2)cc1] CAS: 2659322-56-0 | 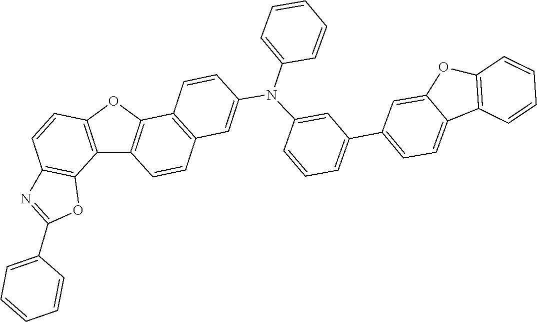 [SMILES: c1ccc(-c2nc3ccc4oc5c6ccc(N(c7ccccc7)c7cccc(-c8ccc9c(c8)oc8ccccc89)c7)cc6ccc5c4c3o2)cc1] | 669.2 | 60 |
| | | 356 | | |
| 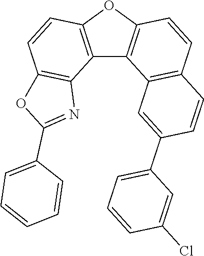 [SMILES: Clc1cccc(-c2ccc3ccc4oc5ccc6oc(-c7ccccc7)nc6c5c4c3c2)c1] Sub-d2 | 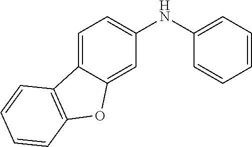 [SMILES: c1ccc(Nc2ccc3c(c2)oc2ccccc23)cc1] CAS: 406488-21-9 | 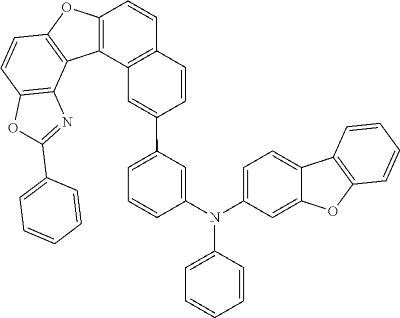 [SMILES: c1ccc(-c2nc3c(ccc4oc5ccc6ccc(-c7cccc(N(c8ccccc8)c8ccc9c(c8)oc8ccccc89)c7)cc6c5c43)o2)cc1] | 669.2 | 58 |
| | | 370 | | |
| 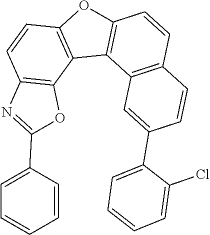 [SMILES: Clc1ccccc1-c1ccc2ccc3oc4ccc5nc(-c6ccccc6)oc5c4c3c2c1] Sub-d3 | 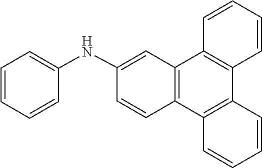 [SMILES: c1ccc(Nc2ccc3c4ccccc4c4ccccc4c3c2)cc1] CAS: 1621976-98-4 | 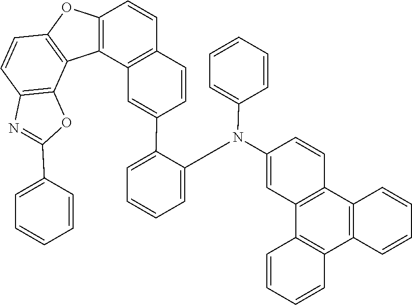 [SMILES: c1ccc(-c2nc3ccc4oc5ccc6ccc(-c7ccccc7N(c7ccccc7)c7ccc8c9ccccc9c9ccccc9c8c7)cc6c5c4c3o2)cc1] | 729.2 | 49 |
| | | 375 | | |
| 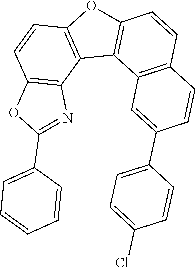 [SMILES: Clc1ccc(-c2ccc3ccc4oc5ccc6oc(-c7ccccc7)nc6c5c4c3c2)cc1] Sub-d4 | 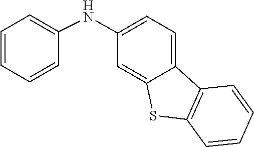 [SMILES: c1ccc(Nc2ccc3c(c2)sc2ccccc23)cc1] CAS: 1427556-53-3 | 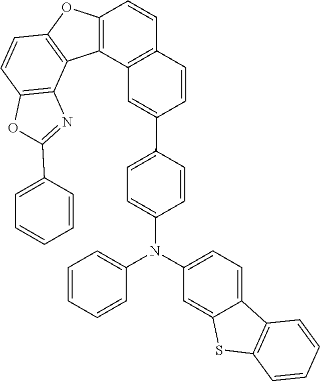 [SMILES: c1ccc(-c2nc3c(ccc4oc5ccc6ccc(-c7ccc(N(c8ccccc8)c8ccc9c(c8)sc8ccccc89)cc7)cc6c5c43)o2)cc1] | 685.2 | 54 |
| | | 390 | | |
| 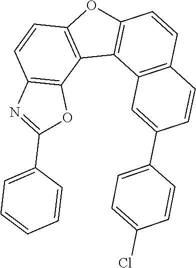 [SMILES: Clc1ccc(-c2ccc3ccc4oc5ccc6nc(-c7ccccc7)oc6c5c4c3c2)cc1] Sub-d1 | 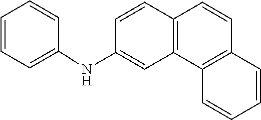 [SMILES: c1ccc(Nc2ccc3ccc4ccccc4c3c2)cc1] CAS: 1357009-66-5 | 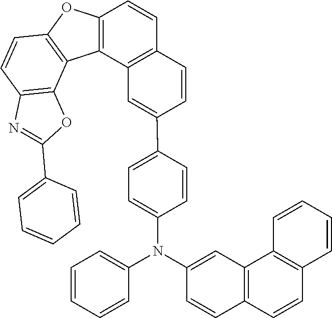 [SMILES: c1ccc(-c2nc3ccc4oc5ccc6ccc(-c7ccc(N(c8ccccc8)c8ccc9ccc%10ccccc%10c9c8)cc7)cc6c5c4c3o2)cc1] | 679.2 | 60 |
| | | 391 | | |
| 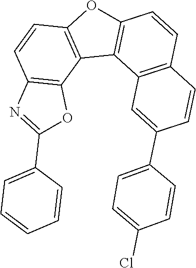 [SMILES: Clc1ccc(-c2ccc3ccc4oc5ccc6nc(-c7ccccc7)oc6c5c4c3c2)cc1] Sub-d1 | 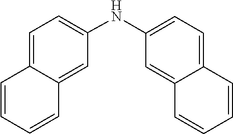 [SMILES: c1ccc2cc(Nc3ccc4ccccc4c3)ccc2c1] CAS: 532-18-3 | 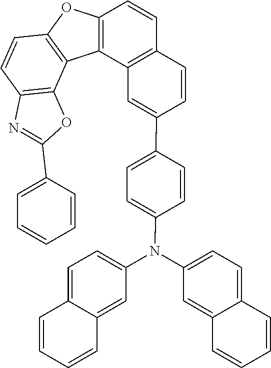 [SMILES: c1ccc(-c2nc3ccc4oc5ccc6ccc(-c7ccc(N(c8ccc9ccccc9c8)c8ccc9ccccc9c8)cc7)cc6c5c4c3o2)cc1] | 679.2 | 53 |
| | | 403 | | |
| 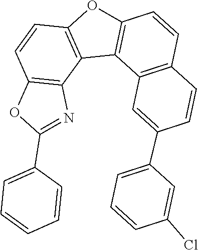 [SMILES: Clc1cccc(-c2ccc3ccc4oc5ccc6oc(-c7ccccc7)nc6c5c4c3c2)c1] Sub-d2 | 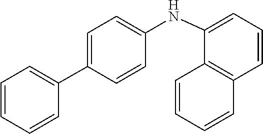 [SMILES: c1ccc(-c2ccc(Nc3cccc4ccccc34)cc2)cc1] CAS: 446242-37-1 | 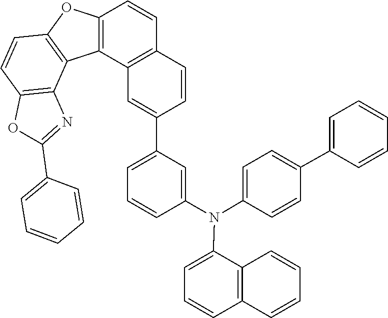 [SMILES: c1ccc(-c2ccc(N(c3cccc(-c4ccc5ccc6oc7ccc8oc(-c9ccccc9)nc8c7c6c5c4)c3)c3cccc4ccccc34)cc2)cc1] | 705.2 | 52 |
| | | 404 | | |
| 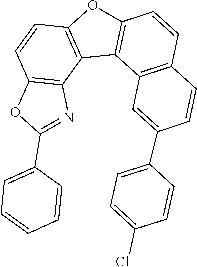 [SMILES: Clc1ccc(-c2ccc3ccc4oc5ccc6oc(-c7ccccc7)nc6c5c4c3c2)cc1] Sub-d4 | 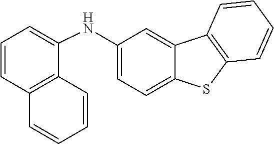 [SMILES: c1ccc2c(Nc3ccc4sc5ccccc5c4c3)cccc2c1] CAS: 1429933-57-2 | 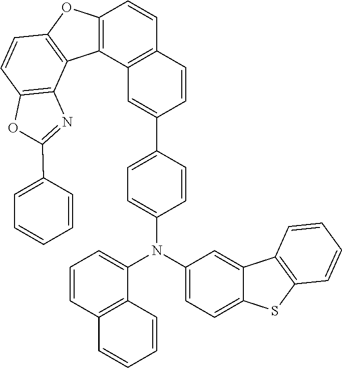 [SMILES: c1ccc(-c2nc3c(ccc4oc5ccc6ccc(-c7ccc(N(c8ccc9sc%10ccccc%10c9c8)c8cccc9ccccc89)cc7)cc6c5c43)o2)cc1] | 735.2 | 60 |
| | | 420 | | |
| 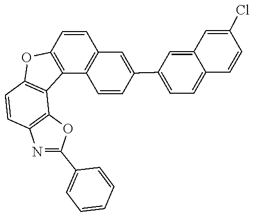 [SMILES: Clc1ccc2ccc(-c3ccc4c(ccc5oc6ccc7nc(-c8ccccc8)oc7c6c54)c3)cc2c1] Sub-d7 | 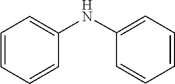 [SMILES: c1ccc(Nc2ccccc2)cc1] CAS: 122-39-4 | 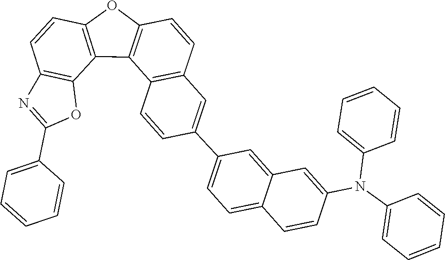 [SMILES: c1ccc(-c2nc3ccc4oc5ccc6cc(-c7ccc8ccc(N(c9ccccc9)c9ccccc9)cc8c7)ccc6c5c4c3o2)cc1] | 629.2 | 64 |
| | | 460 | | |
| 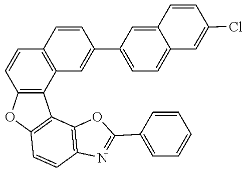 [SMILES: Clc1ccc2cc(-c3ccc4ccc5oc6ccc7nc(-c8ccccc8)oc7c6c5c4c3)ccc2c1] Sub-d5 | 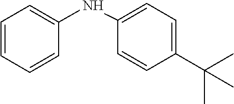 [SMILES: CC(C)(C)c1ccc(Nc2ccccc2)cc1] CAS: 4496-49-5 | 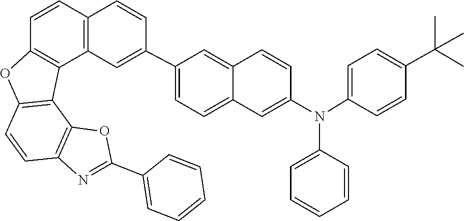 [SMILES: CC(C)(C)c1ccc(N(c2ccccc2)c2ccc3cc(-c4ccc5ccc6oc7ccc8nc(-c9ccccc9)oc8c7c6c5c4)ccc3c2)cc1] | 685.3 | 58 |
| | | 462 | | |
| 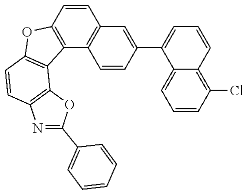 [SMILES: Clc1cccc2c(-c3ccc4c(ccc5oc6ccc7nc(-c8ccccc8)oc7c6c54)c3)cccc12] Sub-d8 | 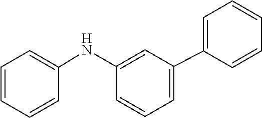 [SMILES: c1ccc(Nc2cccc(-c3ccccc3)c2)cc1] CAS: 198275-79-5 | 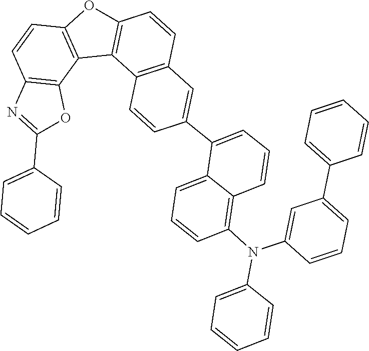 [SMILES: c1ccc(-c2cccc(N(c3ccccc3)c3cccc4c(-c5ccc6c(ccc7oc8ccc9nc(-c%10ccccc%10)oc9c8c76)c5)cccc34)c2)cc1] | 705.3 | 52 |
| | | 465 | | |
| 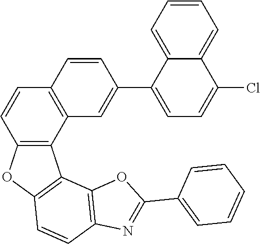 [SMILES: Clc1ccc(-c2ccc3ccc4oc5ccc6nc(-c7ccccc7)oc6c5c4c3c2)c2ccccc12] Sub-d6 | 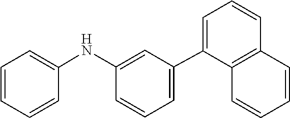 [SMILES: c1ccc(Nc2cccc(-c3cccc4ccccc34)c2)cc1] CAS: 1222634-07-2 | 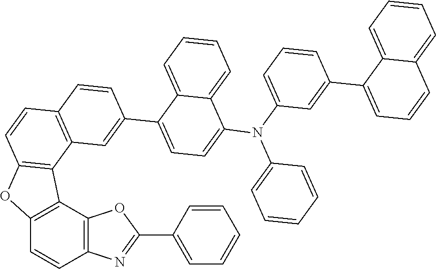 [SMILES: c1ccc(-c2nc3ccc4oc5ccc6ccc(-c7ccc(N(c8ccccc8)c8cccc(-c9cccc%10ccccc9%10)c8)c8ccccc78)cc6c5c4c3o2)cc1] | 755.3 | 53 |
| | | 479 | | |

Compound 130 nuclear magnetism: ¹H-NMR (400 MHz, CD₂Cl₂) δ ppm: 8.35 (d, 2H), 8.06 (d, 1H), 7.99 (d, 1H), 7.89 (d, 1H), 7.86-7.72 (m, 7H), 7.65 (d, 1H), 7.60-7.44 (m, 7H), 7.42-7.34 (m, 5H), 7.26 (d, 1H), 7.05 (s, 1H), 6.85 (d, 1H), 6.73 (d, 1H), 6.42 (d, 1H);

Compound 348 nuclear magnetism: ¹H-NMR (400 MHz, CD₂Cl₂) δ ppm: 8.38-8.26 (m, 5H), 8.06 (d, 1H), 7.97 (d, 1H), 7.85-7.81 (m, 2H), 7.78-7.70 (m, 3H), 7.63-7.46 (m, 5H), 7.43-7.35 (m, 2H), 7.26 (t, 2H), 6.87 (s, 1H), 6.67 (d, 1H), 6.62 (t, 1H), 6.36 (d, 2H).

### Manufacturing and Evaluation of Organic Electroluminescent Devices:

### Example 1: Manufacturing of a Red Organic Electroluminescent Device

First, an anode was pre-treated through the following process: on a ITO/Ag/ITO substrate with thicknesses of 100 Å, 1000 Å, and 100 Å, respectively, surface treatment was performed using ultraviolet ozone and O₂:N₂ plasma to increase a work function of the anode, and the surface of the ITO substrate was cleaned with an organic solvent to remove impurities and oil stains thereon.

PD was evaporated in vacuum on the test substrate (anode) to form a hole injection layer (HIL) having a thickness of 100 Å, and then α-NPD was evaporated in vacuum on the hole injection layer to form a hole transport layer having a thickness of 980 Å.

Compound HT-1 was evaporated in vacuum on the hole transport layer to form a hole adjustment layer having a thickness of 870 Å.

Next, Compound 3, RH-N, and RD-1 were co-evaporated at a rate of 49%:49%:2% on the hole adjustment layer to form a red electroluminescent layer (EML) having a thickness of 400 Å.

The compound ET-1 and LiQ were mixed in a weight ratio of 1:1 and evaporated on the electroluminescent layer to form an electron transport layer (ETL) having a thickness of 350 Å, Yb was evaporated on the electron transport layer to form an electron injection layer (EIL) having a thickness of 10 Å, then magnesium (Mg) and silver (Ag) were mixed at an evaporation rate of 1:9 and evaporated in vacuum on the electron injection layer to form a cathode having a thickness of 130 Å.

In addition, CP having a thickness of 800 Å was evaporated in vacuum on the cathode to complete the manufacturing of the red organic electroluminescent device.

### Examples 2 to 40

Except that Compound 3 in Example 1 was replaced by Compound X in Table 6 below when the electroluminescent layer was formed, organic electroluminescent devices were manufactured by the same method as in Example 1.

### Comparative Examples 1 to 3

Except that Compound 3 in Example 1 was replaced by Compound A, Compound B, and Compound C respectively when the electroluminescent layer was formed, organic electroluminescent devices were manufactured by the same method as in Example 1.

When the organic electroluminescent devices in the examples and the comparative examples were manufactured, structures of the used compounds were as follows.

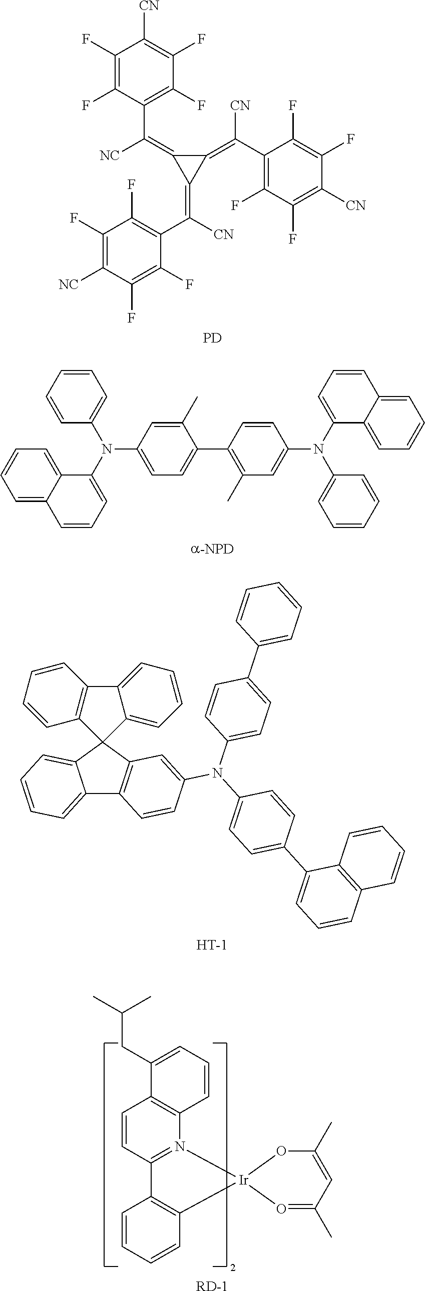

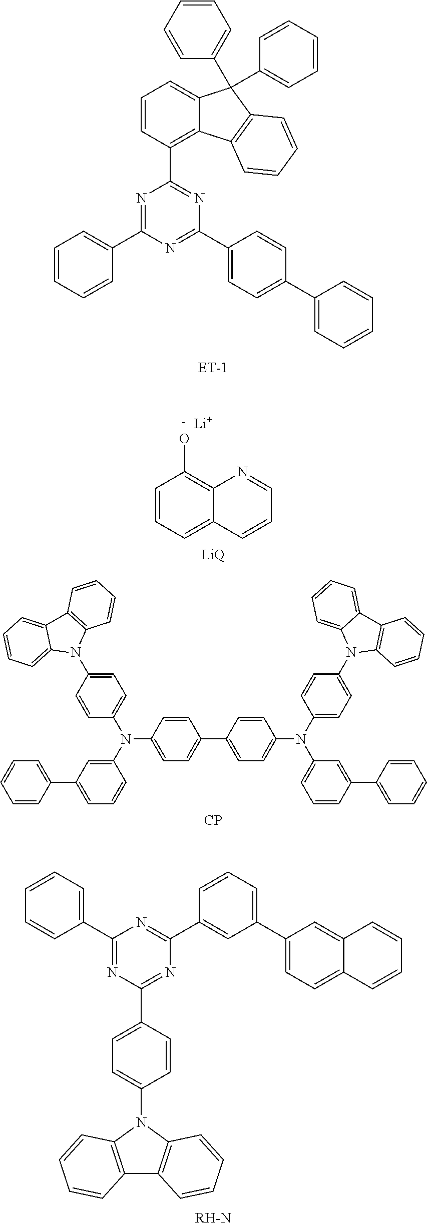

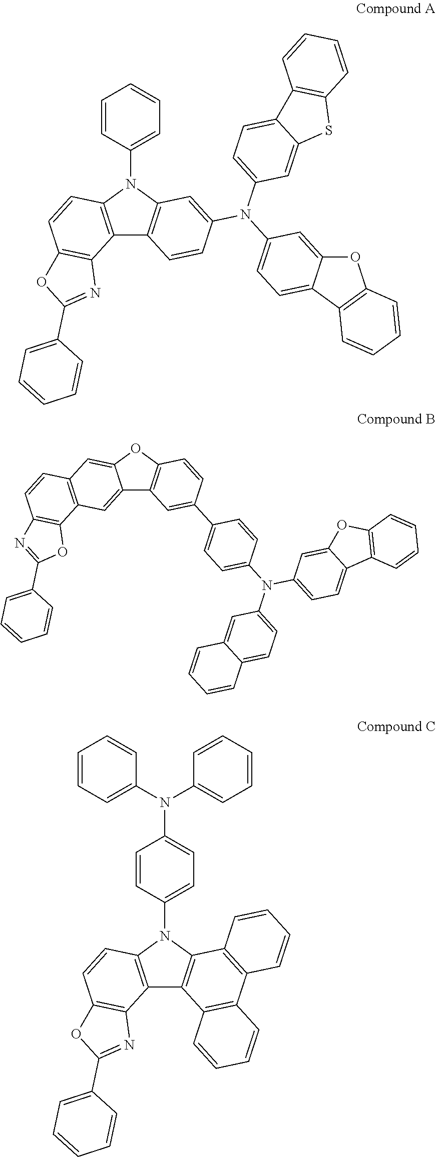

Performances of the red organic electroluminescent devices manufactured in Examples 1 to 40 and Comparative Examples 1 to 3 were tested. Specifically, IVL performance of the devices was tested under the condition of 10 mA/cm², and T95 device life was tested under the condition of 20 mA/cm². See Table 6 for the test results.

TABLE 6

| | | Driving | | | | T95(h) |
|---|---|---|---|---|---|---|
| Example | | voltage | | | | @20 |
| No. | Compound X | (V) | Cd/A | CIEx | CIEy | mA/cm² |
| Example 1 | Compound 3 | 3.42 | 61.7 | 0.680 | 0.320 | 544 |
| Example 2 | Compound 6 | 3.39 | 62.0 | 0.680 | 0.320 | 530 |
| Example 3 | Compound 11 | 3.38 | 62.8 | 0.680 | 0.320 | 518 |
| Example 4 | Compound 14 | 3.39 | 60.7 | 0.680 | 0.320 | 524 |
| Example 5 | Compound 19 | 3.43 | 62.7 | 0.680 | 0.320 | 525 |
| Example 6 | Compound 22 | 3.42 | 62.6 | 0.680 | 0.320 | 536 |
| Example 7 | Compound 29 | 3.40 | 60.8 | 0.680 | 0.320 | 522 |
| Example 8 | Compound 39 | 3.38 | 60.8 | 0.680 | 0.320 | 517 |
| Example 9 | Compound 48 | 3.40 | 62.3 | 0.680 | 0.320 | 536 |
| Example 10 | Compound 52 | 3.37 | 61.7 | 0.680 | 0.320 | 524 |
| Example 11 | Compound 76 | 3.43 | 60.8 | 0.680 | 0.320 | 527 |
| Example 12 | Compound 79 | 3.40 | 61.4 | 0.680 | 0.320 | 537 |
| Example 13 | Compound 102 | 3.42 | 62.0 | 0.680 | 0.320 | 531 |
| Example 14 | Compound 130 | 3.38 | 61.0 | 0.680 | 0.320 | 534 |
| Example 15 | Compound 178 | 3.42 | 60.8 | 0.680 | 0.320 | 541 |
| Example 16 | Compound 181 | 3.38 | 61.4 | 0.680 | 0.320 | 538 |
| Example 17 | Compound 206 | 3.41 | 57.7 | 0.680 | 0.320 | 487 |
| Example 18 | Compound 210 | 3.42 | 57.6 | 0.680 | 0.320 | 477 |
| Example 19 | Compound 212 | 3.42 | 57.3 | 0.680 | 0.320 | 483 |
| Example 20 | Compound 216 | 3.41 | 58.0 | 0.680 | 0.320 | 482 |
| Example 21 | Compound 229 | 3.41 | 57.6 | 0.680 | 0.320 | 484 |
| Example 22 | Compound 233 | 3.39 | 58.3 | 0.680 | 0.320 | 478 |
| Example 23 | Compound 252 | 3.41 | 57.8 | 0.680 | 0.320 | 483 |
| Example 24 | Compound 268 | 3.37 | 57.6 | 0.680 | 0.320 | 521 |
| Example 25 | Compound 293 | 3.42 | 59.0 | 0.680 | 0.320 | 526 |
| Example 26 | Compound 327 | 3.37 | 61.2 | 0.680 | 0.320 | 542 |
| Example 27 | Compound 345 | 3.38 | 57.5 | 0.680 | 0.320 | 475 |
| Example 28 | Compound 348 | 3.37 | 57.2 | 0.680 | 0.320 | 485 |
| Example 29 | Compound 356 | 3.40 | 57.4 | 0.680 | 0.320 | 483 |
| Example 30 | Compound 370 | 3.37 | 61.9 | 0.680 | 0.320 | 519 |
| Example 31 | Compound 375 | 3.41 | 60.8 | 0.680 | 0.320 | 530 |
| Example 32 | Compound 390 | 3.43 | 62.4 | 0.680 | 0.320 | 520 |
| Example 33 | Compound 391 | 3.40 | 61.5 | 0.680 | 0.320 | 542 |
| Example 34 | Compound 403 | 3.38 | 62.7 | 0.680 | 0.320 | 531 |
| Example 35 | Compound 404 | 3.41 | 61.1 | 0.680 | 0.320 | 525 |
| Example 36 | Compound 420 | 3.41 | 62.5 | 0.680 | 0.320 | 544 |
| Example 37 | Compound 460 | 3.40 | 57.8 | 0.680 | 0.320 | 471 |
| Example 38 | Compound 462 | 3.41 | 61.9 | 0.680 | 0.320 | 530 |
| Example 39 | Compound 465 | 3.43 | 57.9 | 0.680 | 0.320 | 474 |
| Example 40 | Compound 479 | 3.39 | 62.3 | 0.680 | 0.320 | 518 |
| Comparative | Compound A | 3.45 | 47.9 | 0.68 | 0.32 | 355 |
| Example 1 | | | | | | |
| Comparative | Compound B | 3.47 | 51.3 | 0.68 | 0.32 | 412 |
| Example 2 | | | | | | |
| Comparative | Compound C | 3.46 | 51.7 | 0.68 | 0.32 | 423 |
| Example 3 | | | | | | |

It may be seen from Table 6 above that, when the compound of the present application was used as the host material of the red organic electroluminescent device, the efficiency was improved by at least 10.6% and the service life was prolonged by at least 11.3%.

The reason is that the compound of the present application includes naphthofurano (phenanthofurano) oxazole/thiazole and triarylamine, where arylamine is connected to a naphthalene ring (phenanthrene ring) of a naphthofuran (phenanthofuran) group. After naphthofuran (phenanthofuran) is condensed with oxazole/thiazole, a conjugated system of the compound increases, which helps to stack molecules, thereby significantly enhancing hole transport capacity of the compound in the present application. The compound of the present application is mixed with an electron transport material to form a mixed host material, which can improve carrier balance in the luminescent layer, broaden a carrier recombination region, improve exciton generation and utilization efficiency, improve electroluminescent efficiency of the device, and prolong service life of the device.

The preferred embodiments of the present application are described in detail above with reference to the accompanying drawings. However, the present application is not limited to the specific details of the foregoing embodiments. Multiple simple variations may be made to the technical solutions of the present application within the scope of the technical concept of the invention, and these simple variations fall within the protection scope of the present application.

1. A heterocyclic compound having a structure as shown in Formula 1: 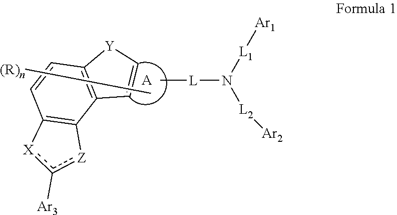
wherein Y is selected from S or O;
one of X and Z is —N═, and the other is O or S;
Ring A is selected from a naphthalene ring or a phenanthrene ring;
L, L₁, and L₂ are the same or different, and are each independently selected from a single bond, a substituted or unsubstituted arylene with 6 to 30 carbon atoms, or a substituted or unsubstituted heteroarylene with 3 to 30 carbon atoms;
Ar₁, Ar₂, and Ar₃ are the same or different, and are each independently selected from a substituted or unsubstituted aryl with 6 to 40 carbon atoms, a substituted or unsubstituted heteroaryl with 3 to 40 carbon atoms, an alkyl with 1 to 10 carbon atoms, or a cycloalkyl with 3 to 10 carbon atoms;
substituents of L, L₁, L₂, Ar₁, Ar₂, and Ar₃ are the same or different, and are each independently selected from deuterium, a cyano, a halogen group, an alkyl with 1 to 10 carbon atoms, a haloalkyl with 1 to 10 carbon atoms, a deuteroalkyl with 1 to 10 carbon atoms, a trialkylsilyl with 3 to 12 carbon atoms, a triphenylsilyl, an aryl with 6 to 20 carbon atoms, a deuteroaryl with 6 to 20 carbon atoms, a heteroaryl with 3 to 20 carbon atoms, a cycloalkyl with 3 to 10 carbon atoms, an alkoxy with 1 to 10 carbon atoms, an alkylthio with 1 to 10 carbon atoms, an aryloxy with 6 to 20 carbon atoms, or an arylthio with 6 to 20 carbon atoms; optionally, any two adjacent substituents form a saturated or unsaturated 3-membered to 15-membered ring; and
each R is the same or different, and is each independently selected from hydrogen, deuterium, a cyano, a halogen group, an alkyl with 1 to 10 carbon atoms, a haloalkyl with 1 to 10 carbon atoms, a deuteroalkyl with 1 to 10 carbon atoms, a trialkylsilyl with 3 to 12 carbon atoms, a triphenylsilyl, an aryl with 6 to 20 carbon atoms, a heteroaryl with 3 to 20 carbon atoms, or a cycloalkyl with 3 to 10 carbon atoms; optionally, any two adjacent Rs forms a ring; and n is selected from 1, 2, 3, 4, 5, 6, 7, 8, or 9.

2. The heterocyclic compound according to claim 1, wherein the compound as shown in Formula 1 has structures as shown in Formulas 1-1 to 1-16:

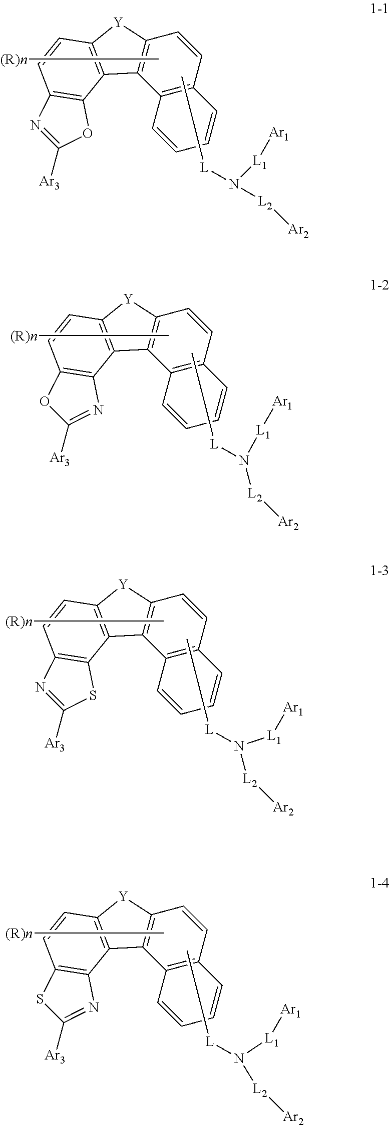

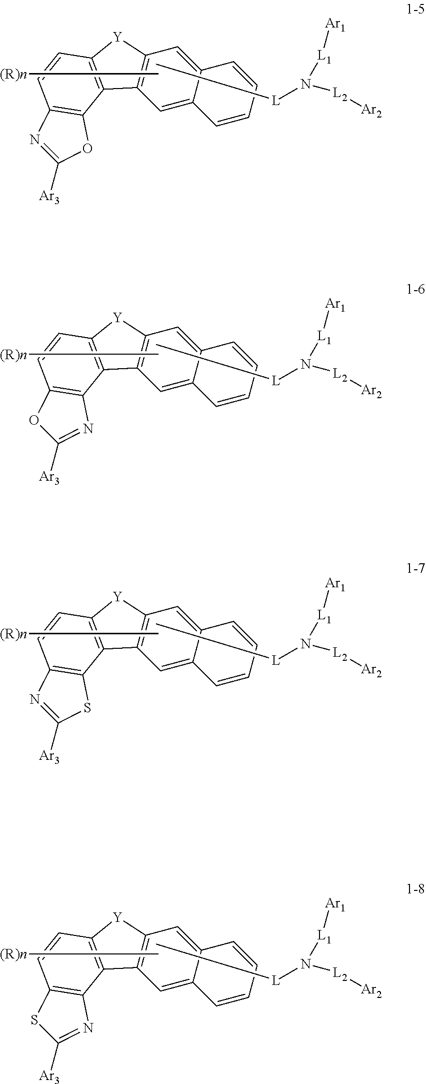

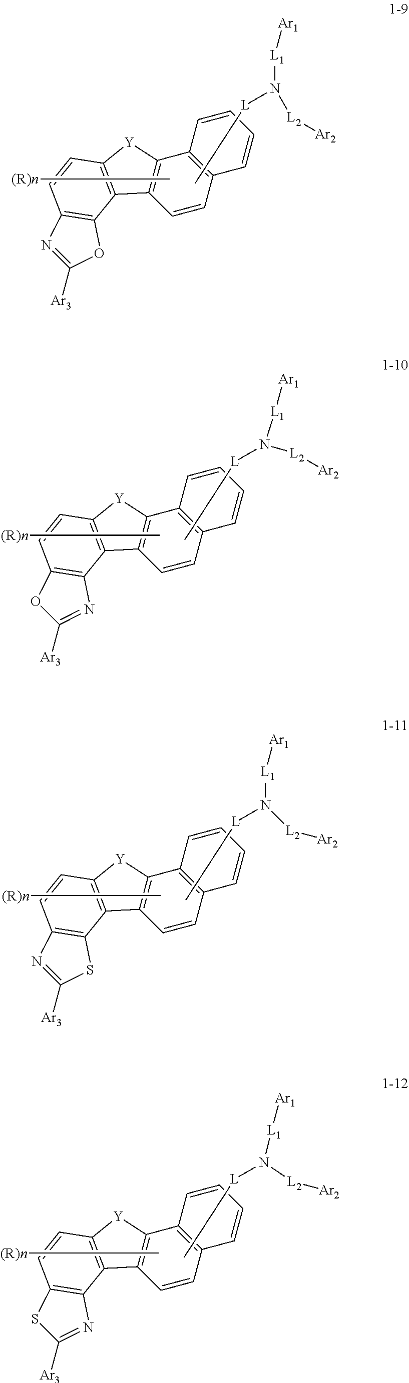

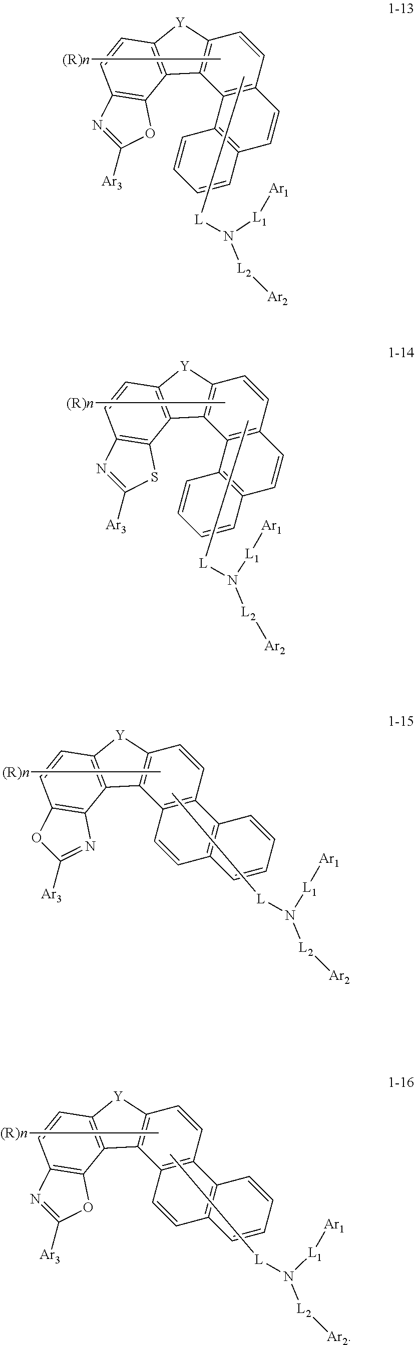

3. The heterocyclic compound according to claim 1, wherein Ar₁, Ar₂, and Ar₃ are the same or different, and are each independently selected from a substituted or unsubstituted aryl with 6 to 25 carbon atoms, or a substituted or unsubstituted heteroaryl with 5 to 24 carbon atoms; and
the substituents of Ar₁, Ar₂, and Ar₃ are independently selected from deuterium, a halogen group, a cyano, a haloalkyl with 1 to 4 carbon atoms, a deuteroalkyl with 1 to 4 carbon atoms, an alkyl with 1 to 4 carbon atoms, a cycloalkyl with 5 to 10 carbon atoms, an aryl with 6 to 15 carbon atoms, a heteroaryl with 5 to 12 carbon atoms, a trialkylsilyl with 3 to 8 carbon atoms, or a deuteroaryl with 6 to 15 carbon atoms, and optionally, any two adjacent substituents form a benzene ring or a fluorene ring.

4. The heterocyclic compound according to claim 1, wherein Ar₁, Ar₂, and Ar₃ are each independently selected from a substituted or unsubstituted group W; the unsubstituted group W is selected from the group consisting of the following groups: 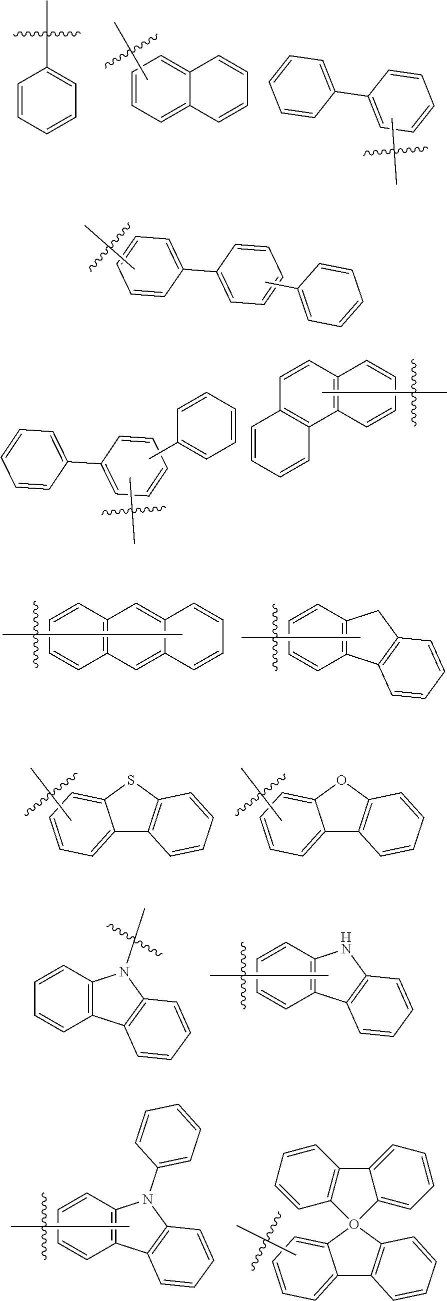 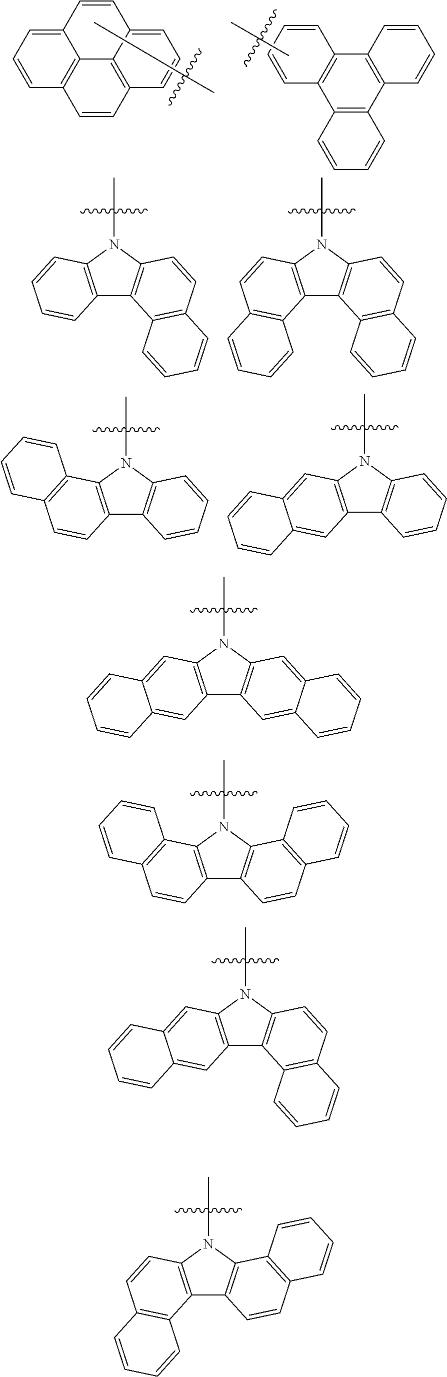 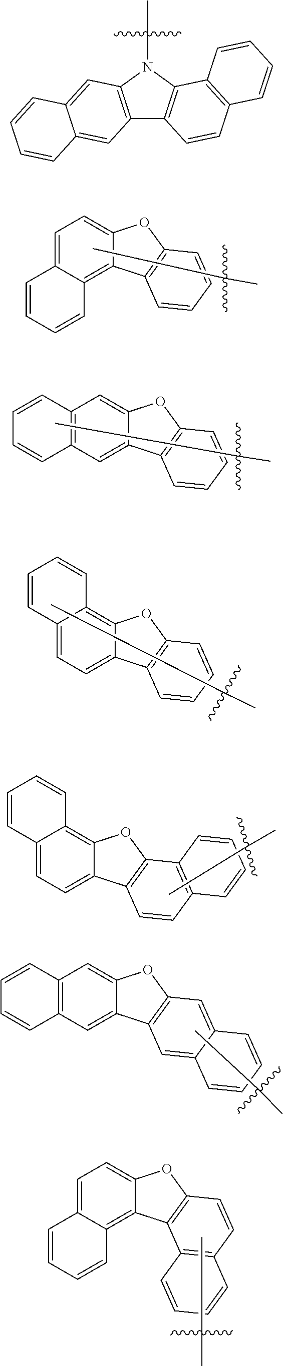 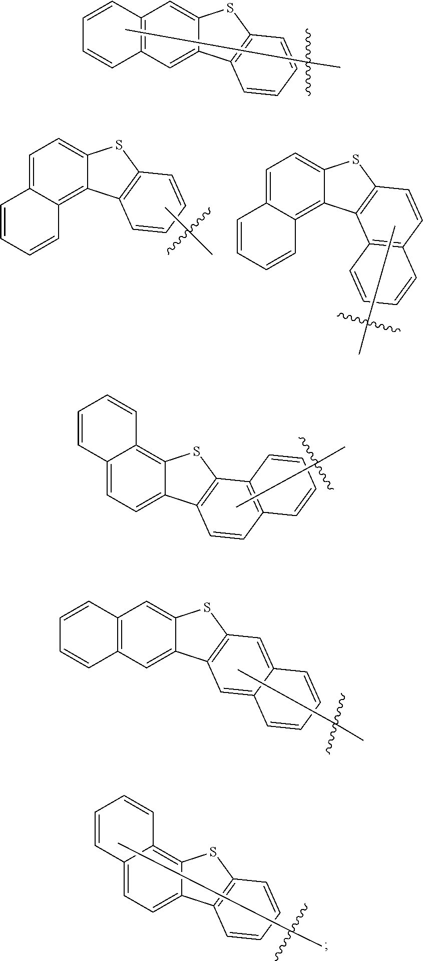
and
the substituted group W has one or more substituents, and the substituents of the substituted group W are each independently selected from deuterium, a fluorine, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a dibenzofuranyl, a dibenzothiophenyl, or a carbazolyl, and when the number of substituents of the group W is greater than 1, each substituent is the same or different.

5. The heterocyclic compound according to claim 1, wherein L, L₁, and L₂ are the same or different, and are each independently selected from a single bond, a substituted or unsubstituted arylene with 6 to 18 carbon atoms, or a substituted or unsubstituted heteroarylene with 5 to 18 carbon atoms; and
the substituents of L, L₁, and L₂ are each independently selected from deuterium, a fluorine, a cyano, an alkyl with 1 to 5 carbon atoms, a trialkylsilyl with 3 to 8 carbon atoms, a fluoroalkyl with 1 to 4 carbon atoms, a deuteratoalkyl with 1 to 4 carbon atoms, a phenyl, or a naphthyl.

6. The heterocyclic compound according to claim 1, wherein L, L₁, and L₂ are each independently selected from a single bond or a substituted or unsubstituted group Q, and the unsubstituted group Q is selected from the group consisting of the following groups: 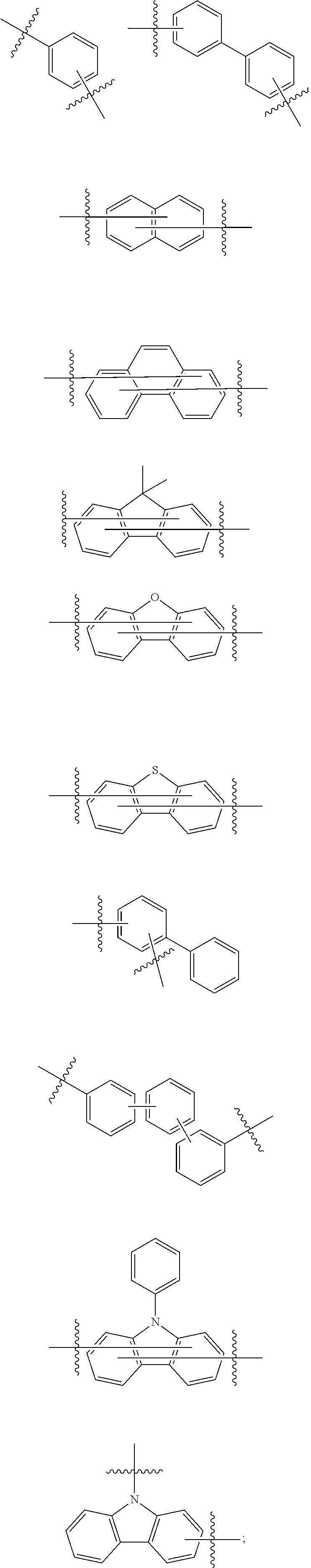
and
the substituted group Q has one or more substituents, and the substituents of the substituted group Q are independently selected from deuterium, a fluorine, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a dibenzofuranyl, a dibenzothiophenyl, or a carbazolyl, and when the number of substituents of the group Q is greater than 1, each substituent is the same or different.

7. The heterocyclic compound according to claim 1, wherein Ar₃ is selected from the group consisting of the following groups:

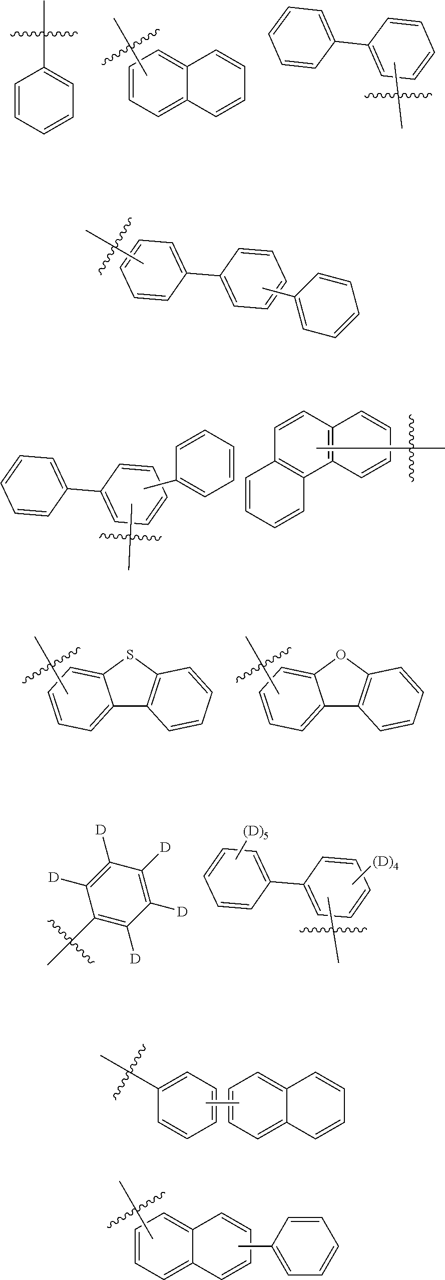

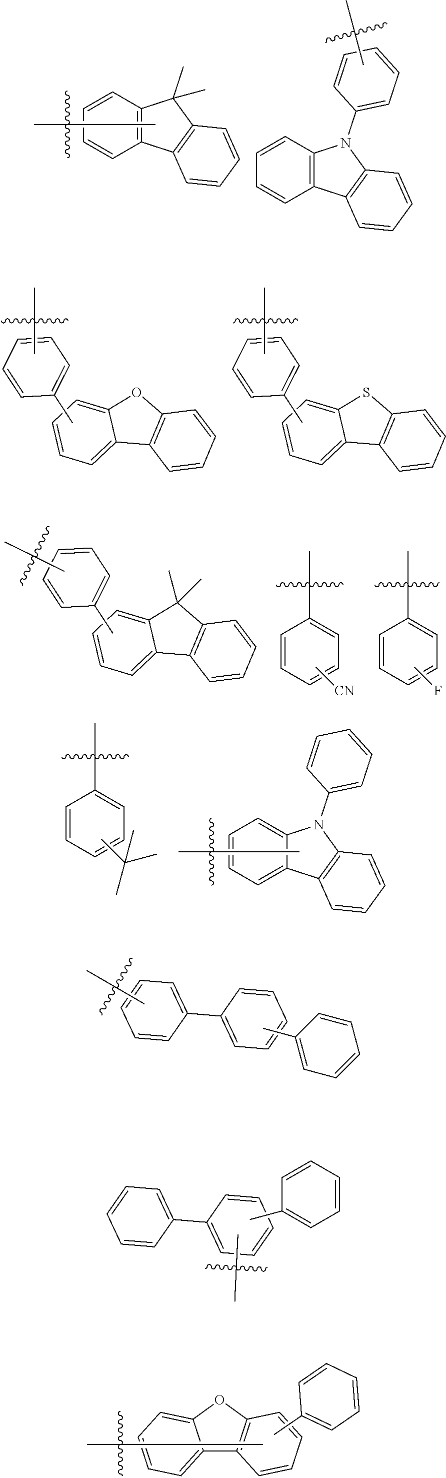

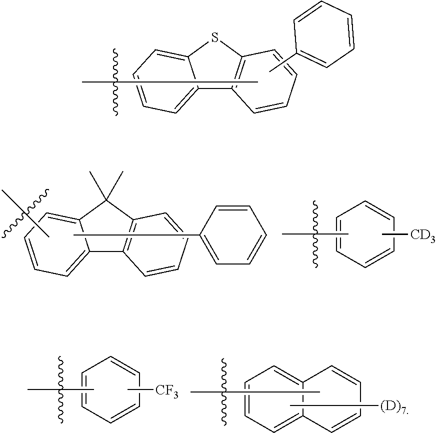

8. The heterocyclic compound according to claim 1, wherein 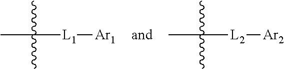
are each independently selected from the group consisting of the following groups:

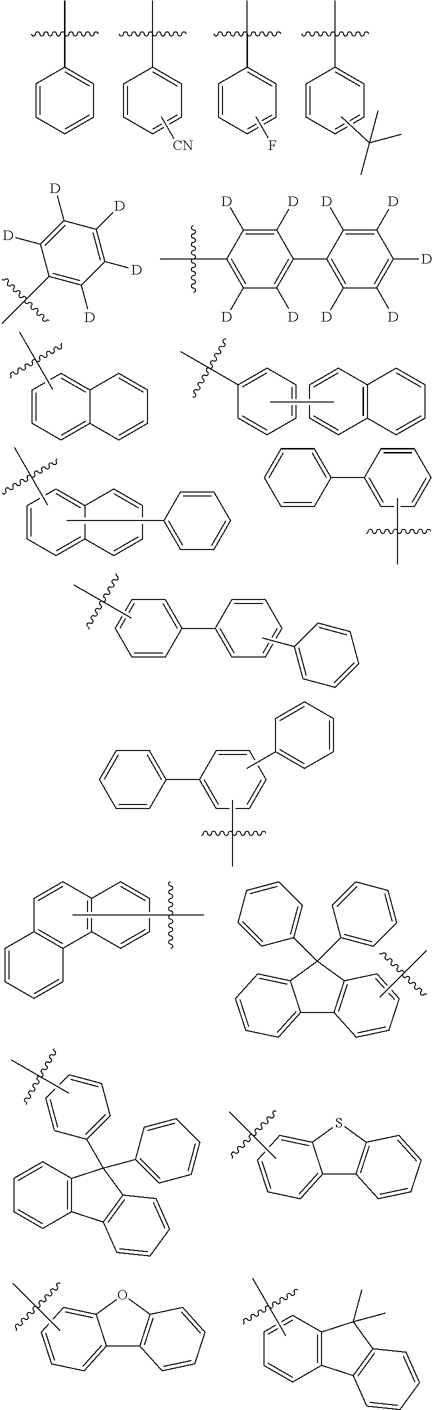

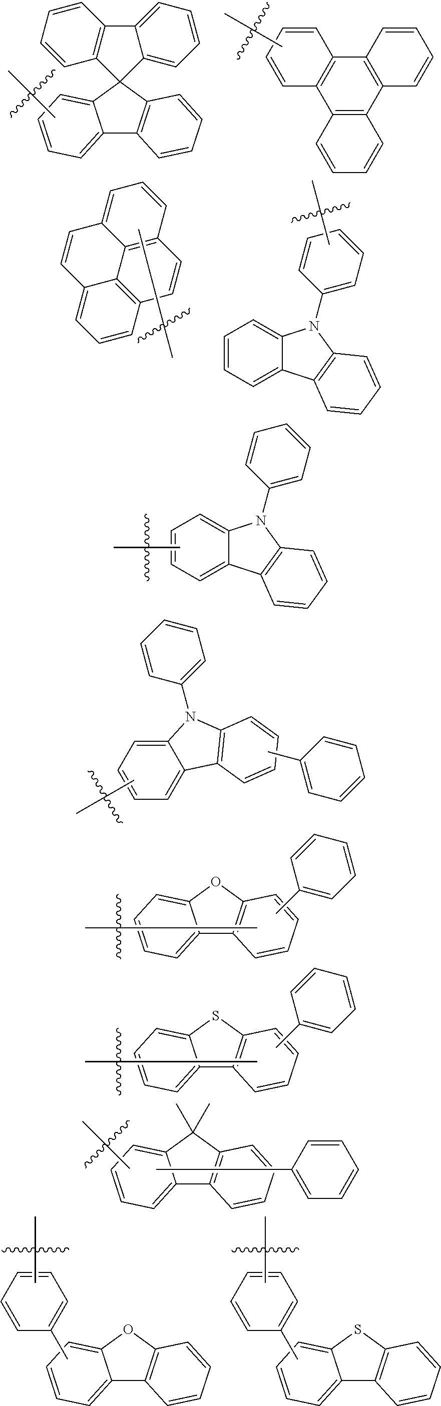

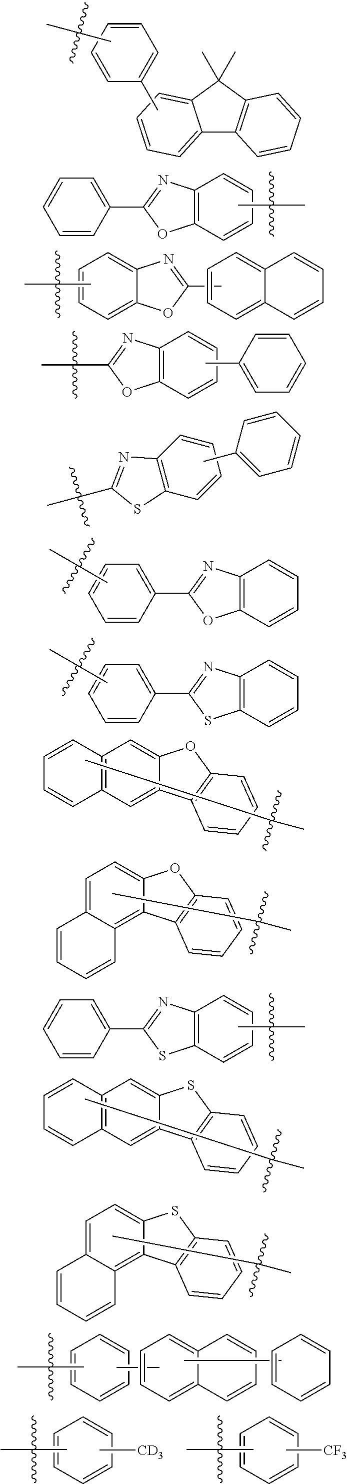

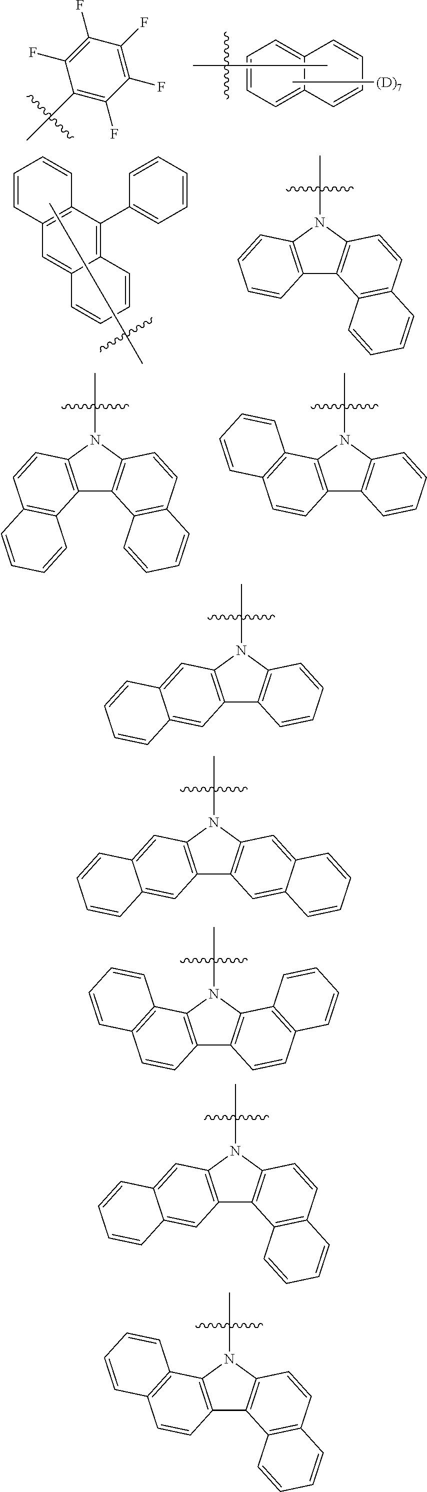

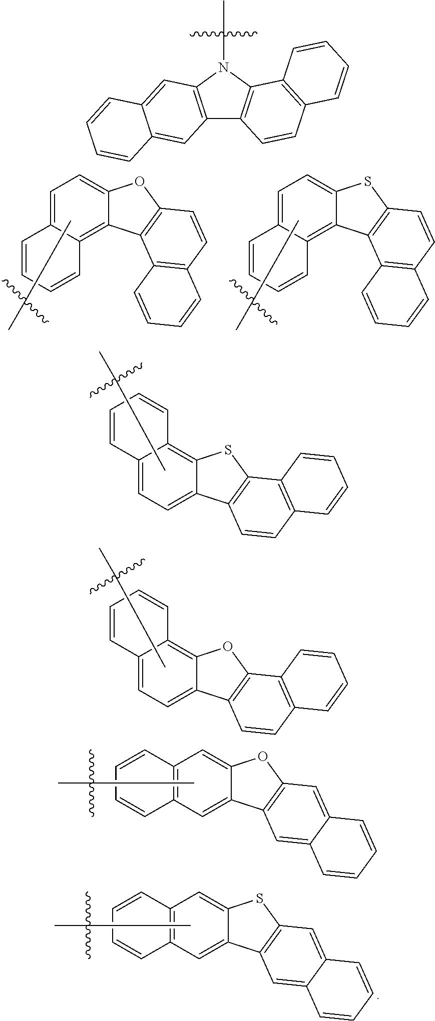

9. The heterocyclic compound according to claim 1, wherein L is selected from a single bond or the group consisting of the following groups: 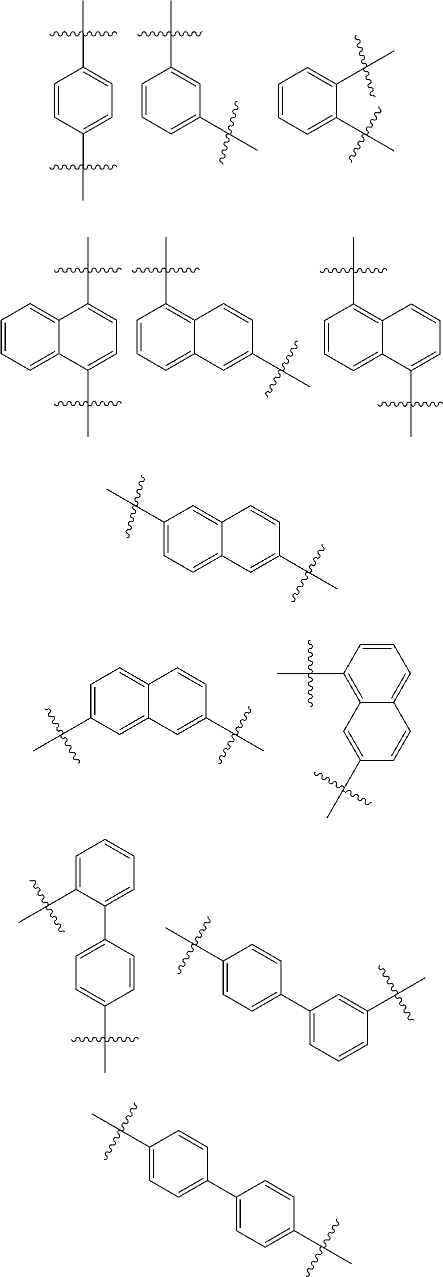 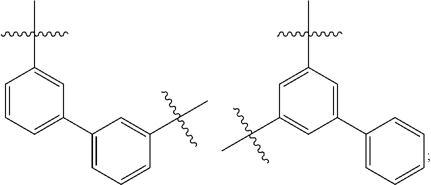 [SMILES: *c1cccc(-c2cccc(*)c2)c1 ; *c1cc(*)cc(-c2ccccc2)c1]
and
L₁ and L₂ are each independently selected from a single bond or the group consisting of the following groups:

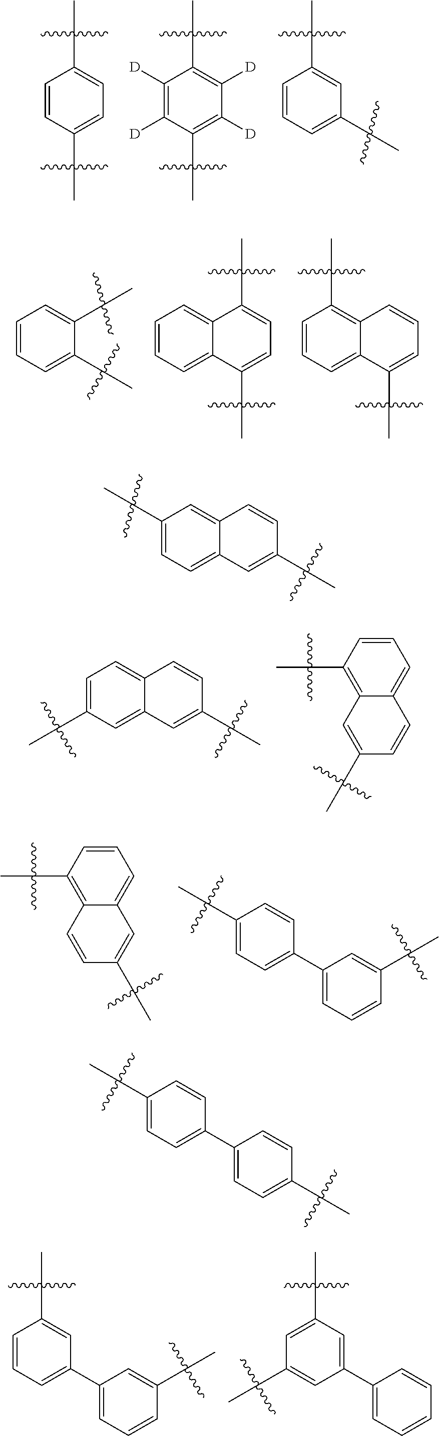

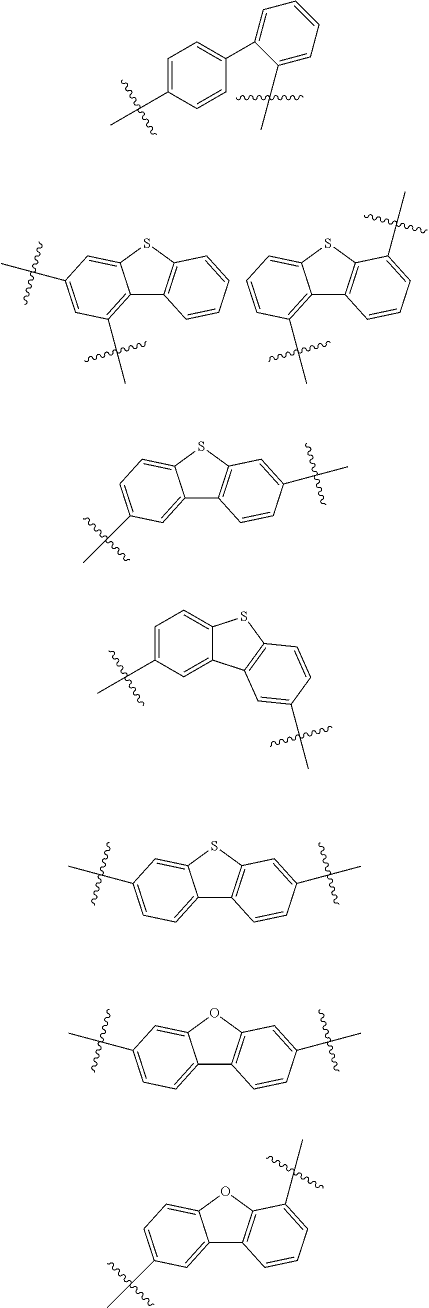

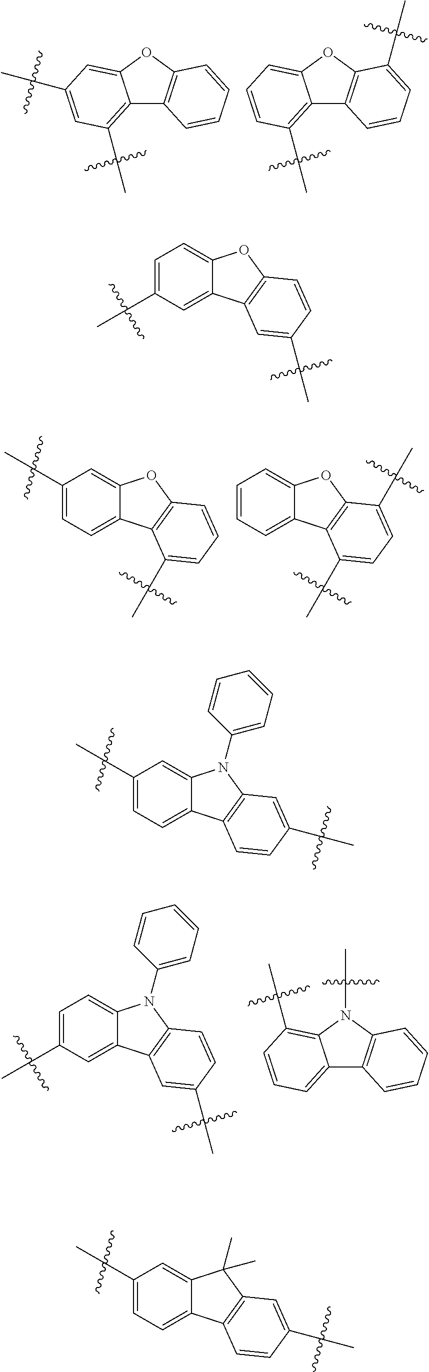

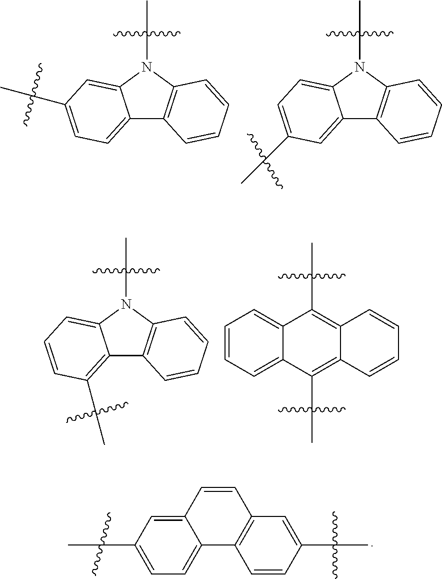

10. The heterocyclic compound according to claim 1, wherein Ar₁ and Ar₂ are each independently selected from the group consisting of the following groups: 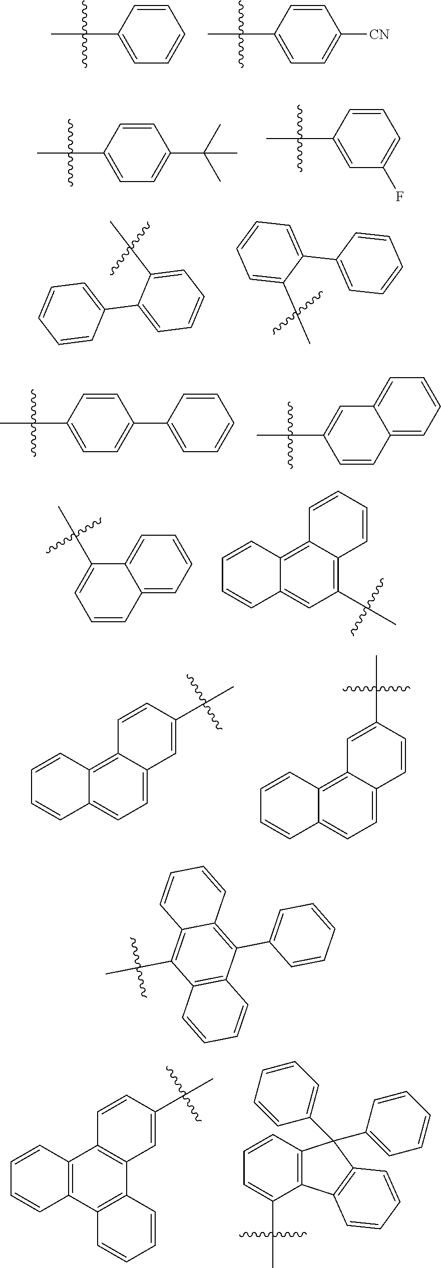 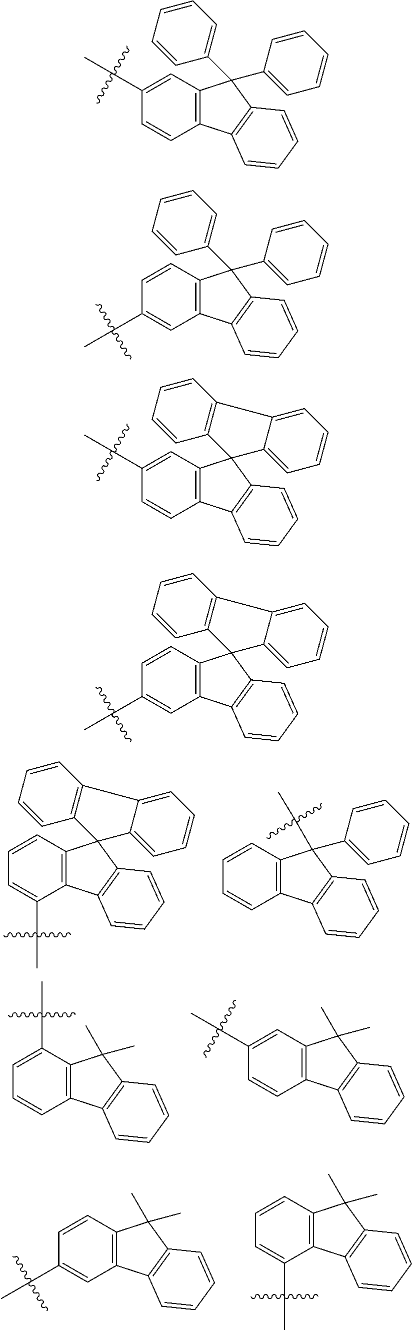 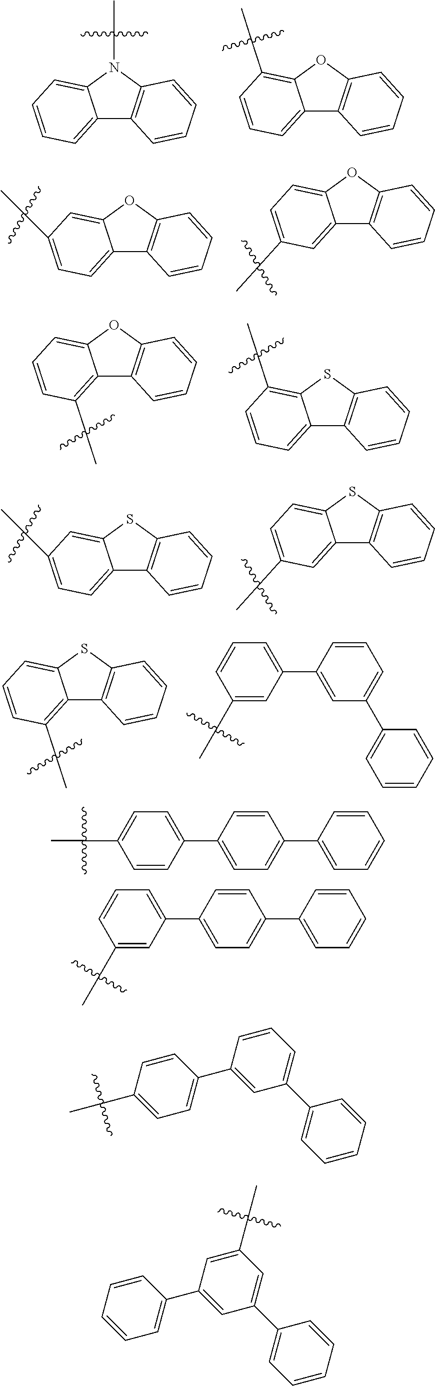 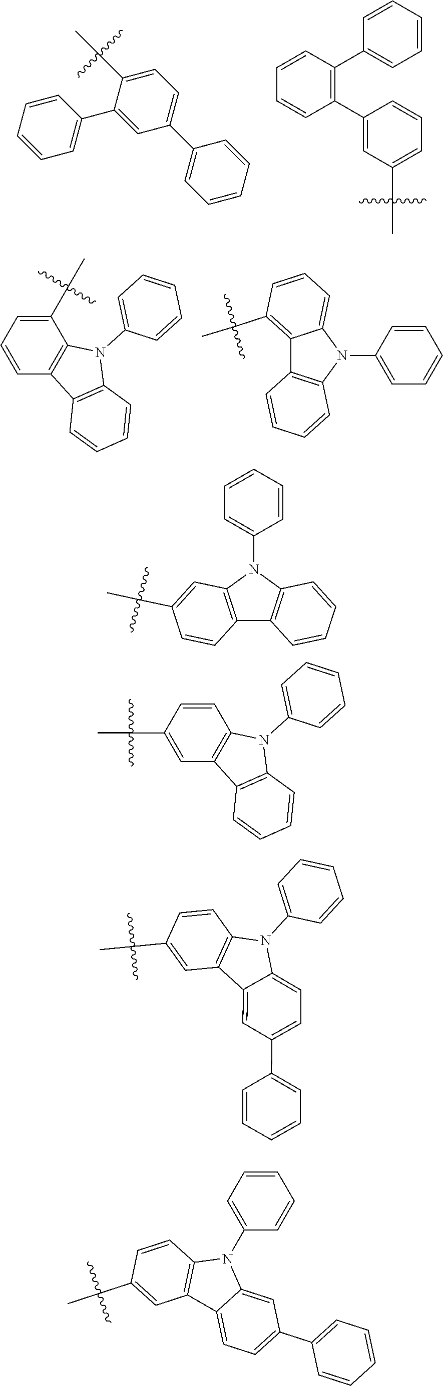 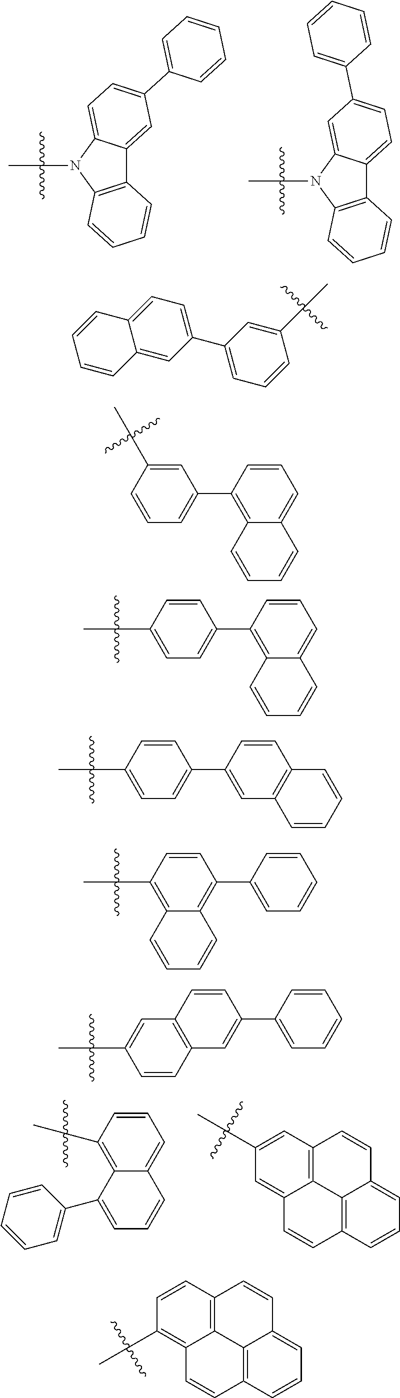 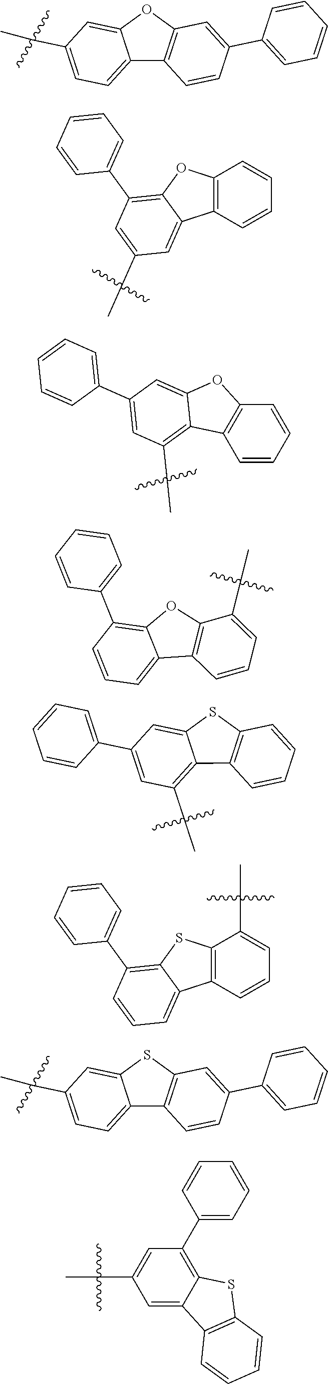 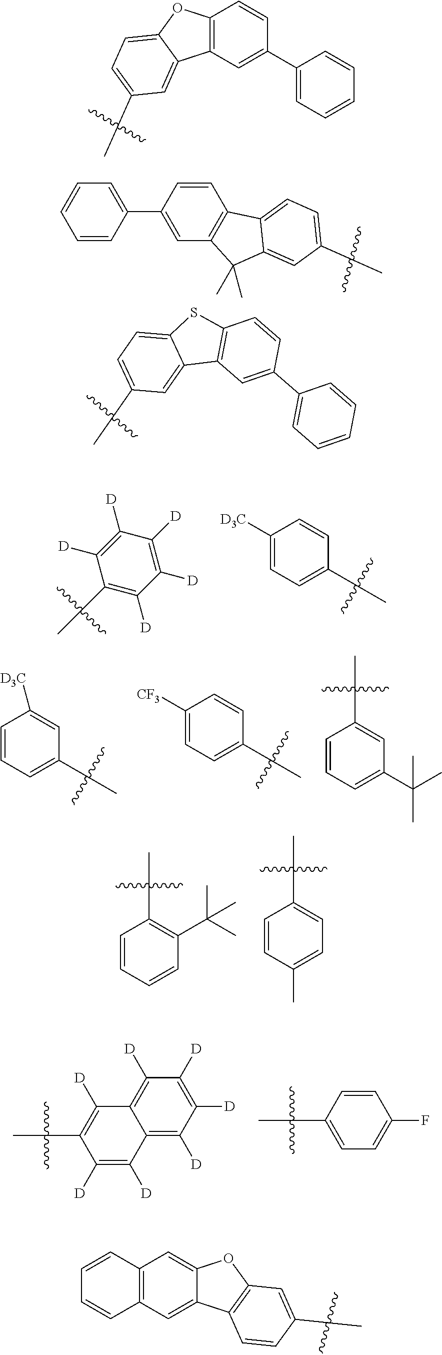 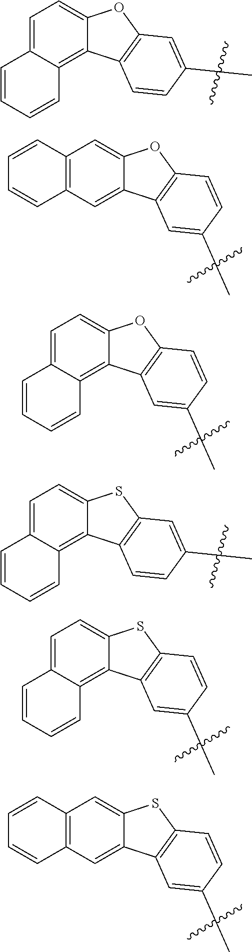 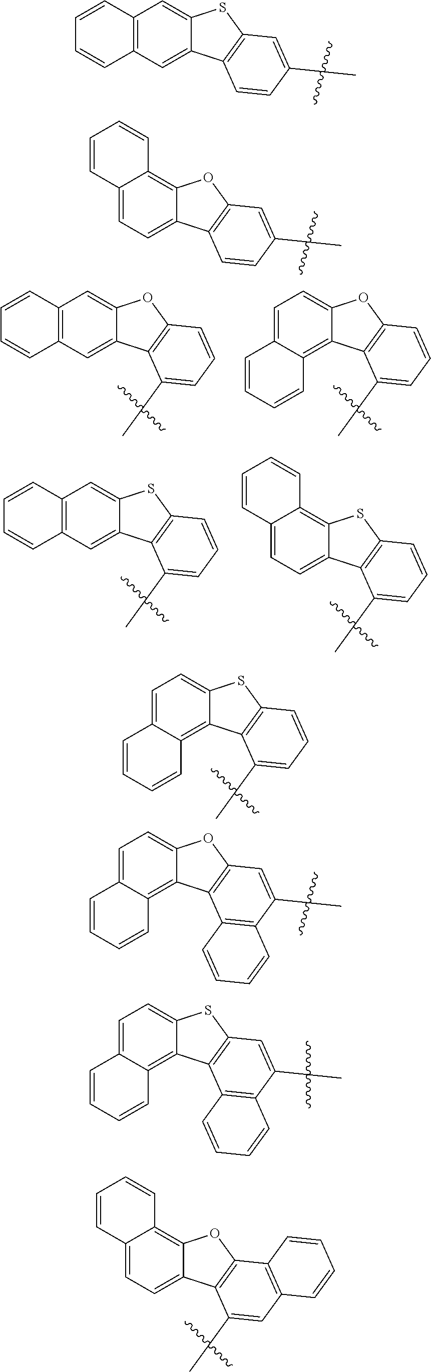 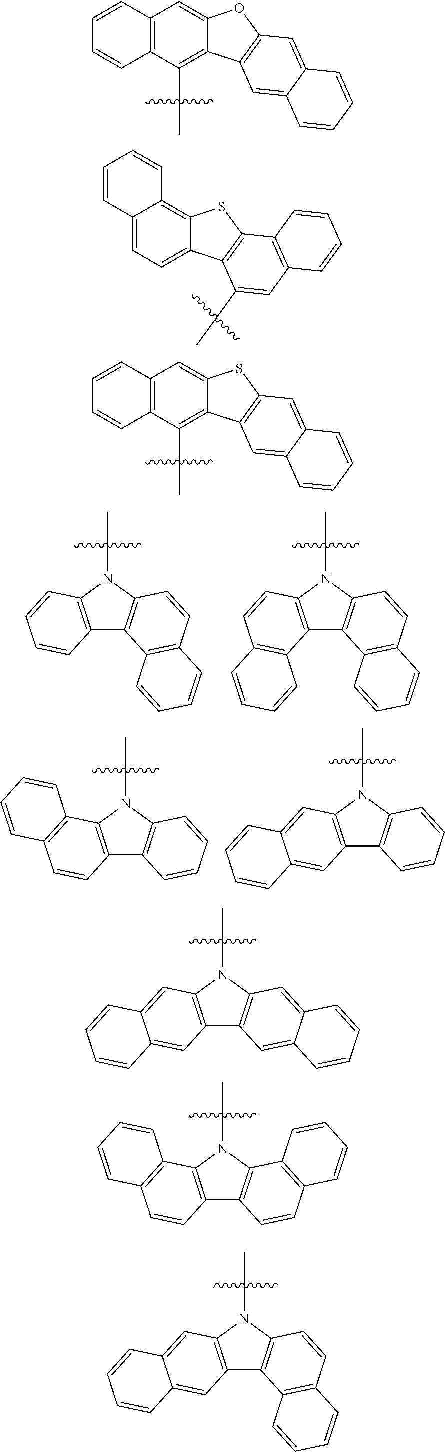 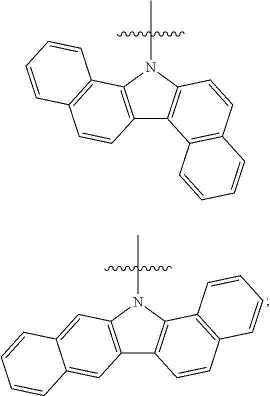 [SMILES: *n1c2ccc3ccccc3c2c2ccc3ccccc3c21 ; *n1c2cc3ccccc3cc2c2ccc3ccccc3c21]
and
Ar₃ is selected from the group consisting of the following groups:

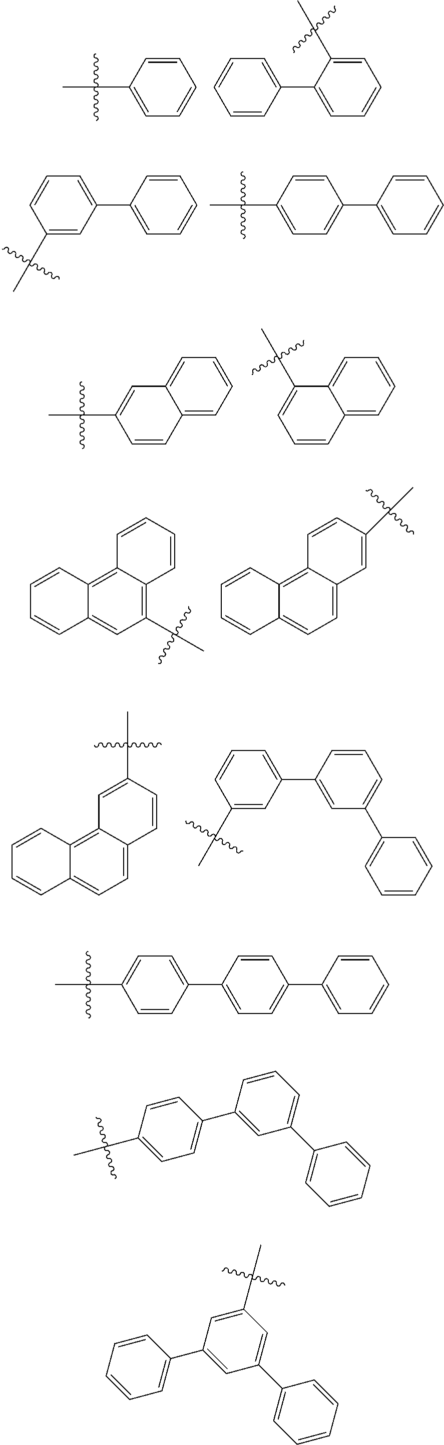

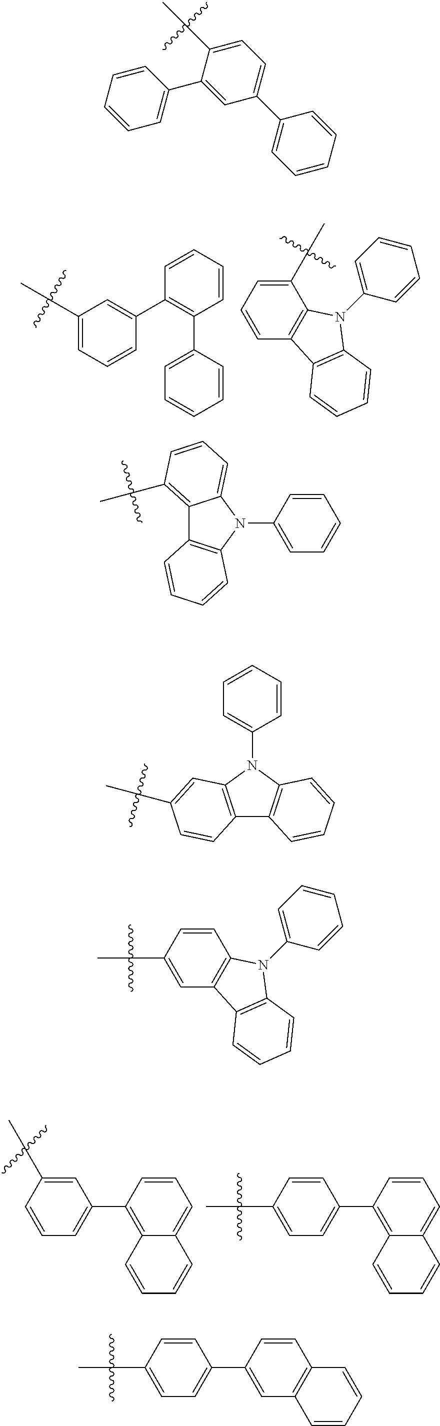

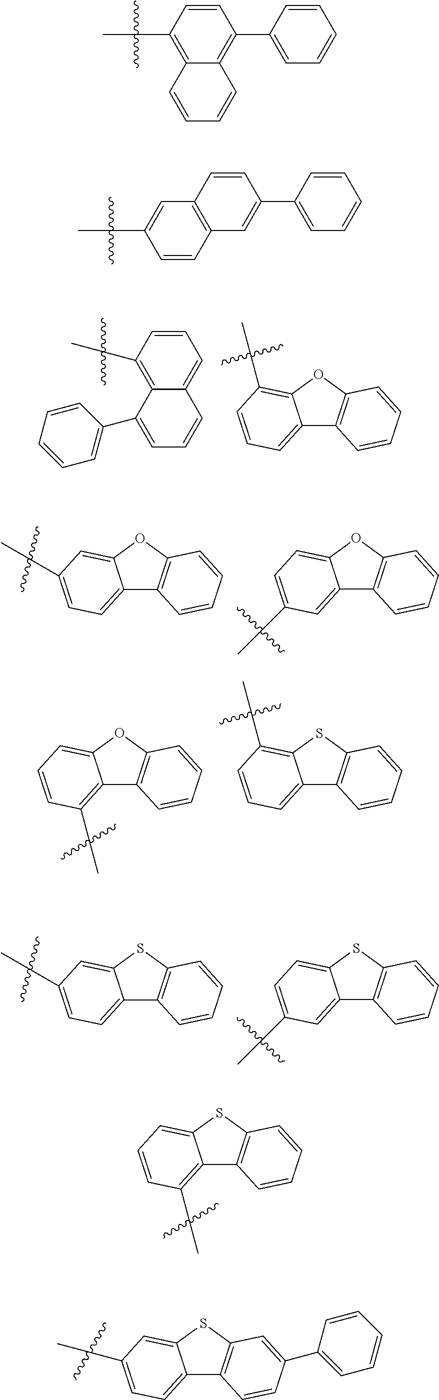

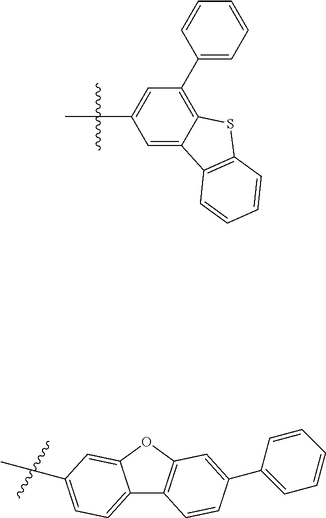 [SMILES: *c1cc(-c2ccccc2)c2sc3ccccc3c2c1 ; *c1ccc2c(c1)oc1cc(-c3ccccc3)ccc12]

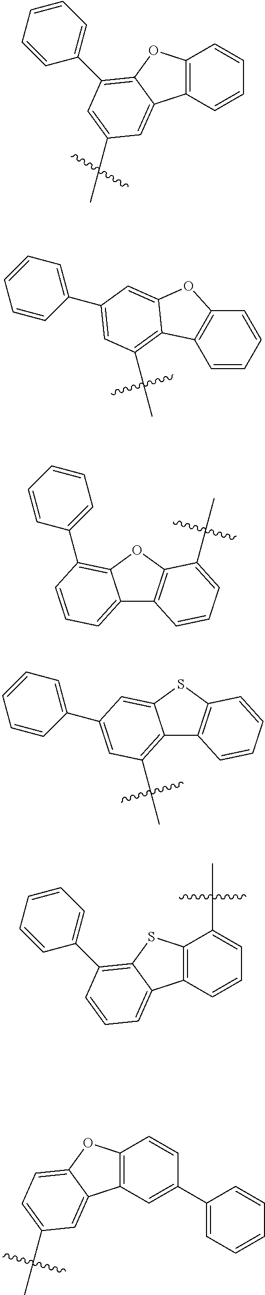 [SMILES: *c1cc(-c2ccccc2)c2oc3ccccc3c2c1 ; *c1cc(-c2ccccc2)cc2oc3ccccc3c12 ; *c1cccc2c1oc1c(-c3ccccc3)cccc12 ; *c1cc(-c2ccccc2)cc2sc3ccccc3c12 ; *c1cccc2c1sc1c(-c3ccccc3)cccc12 ; *c1ccc2oc3ccc(-c4ccccc4)cc3c2c1]

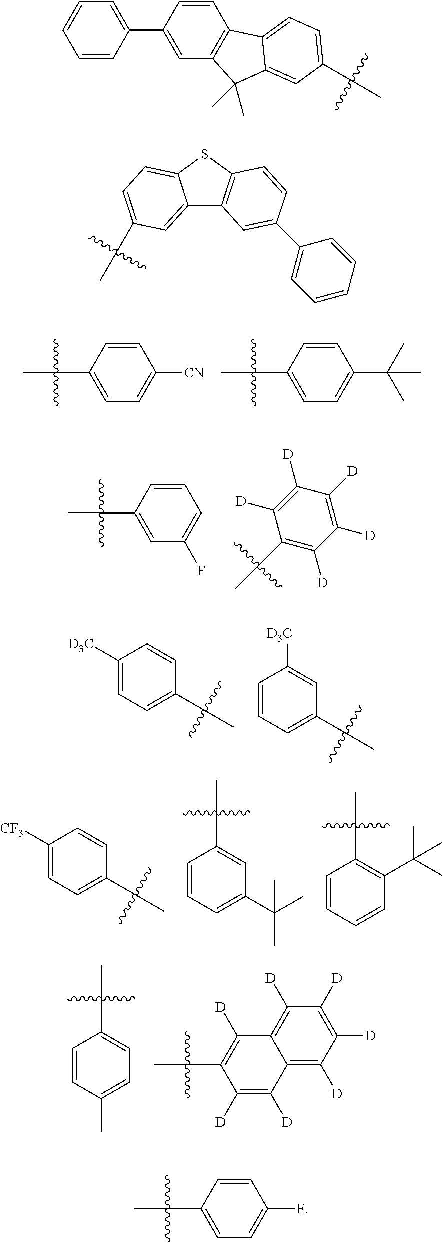

11. The heterocyclic compound according to claim 1, wherein each R is the same or different, and is each independently selected from deuterium, a cyano, a fluorine, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a dibenzofuranyl, a dibenzothiophenyl, or a carbazoyl; and optionally, any two adjacent substituents form a benzene ring.

12. The heterocyclic compound according to claim 1, wherein the heterocyclic compound is selected from the group consisting of the following compounds:

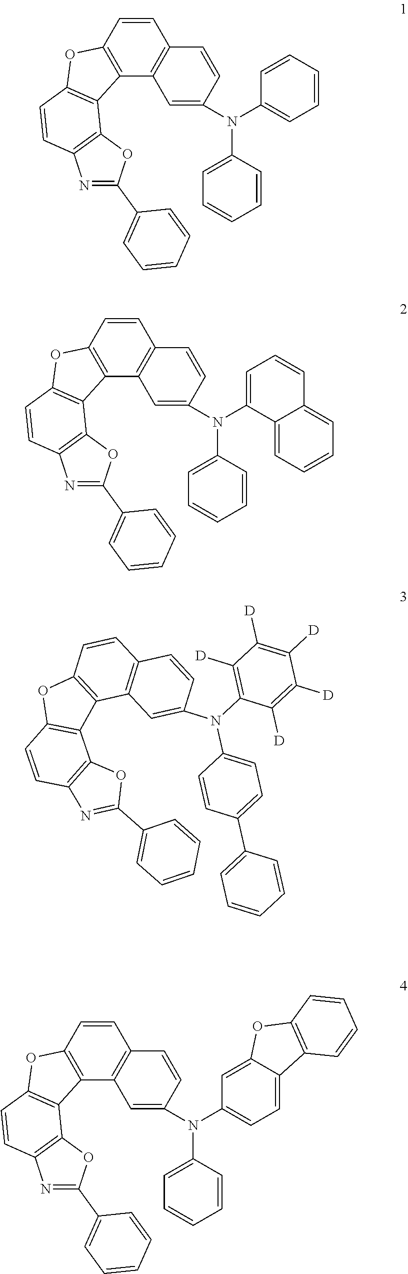 [SMILES: c1ccc(-c2nc3ccc4oc5ccc6ccc(N(c7ccccc7)c7ccccc7)cc6c5c4c3o2)cc1 ; c1ccc(-c2nc3ccc4oc5ccc6ccc(N(c7ccccc7)c7cccc8ccccc78)cc6c5c4c3o2)cc1 ; [2H]c1c([2H])c([2H])c(N(c2ccc(-c3ccccc3)cc2)c2ccc3ccc4oc5ccc6nc(-c7ccccc7)oc6c5c4c3c2)c([2H])c1[2H] ; c1ccc(-c2nc3ccc4oc5ccc6ccc(N(c7ccccc7)c7ccc8c(c7)oc7ccccc78)cc6c5c4c3o2)cc1]

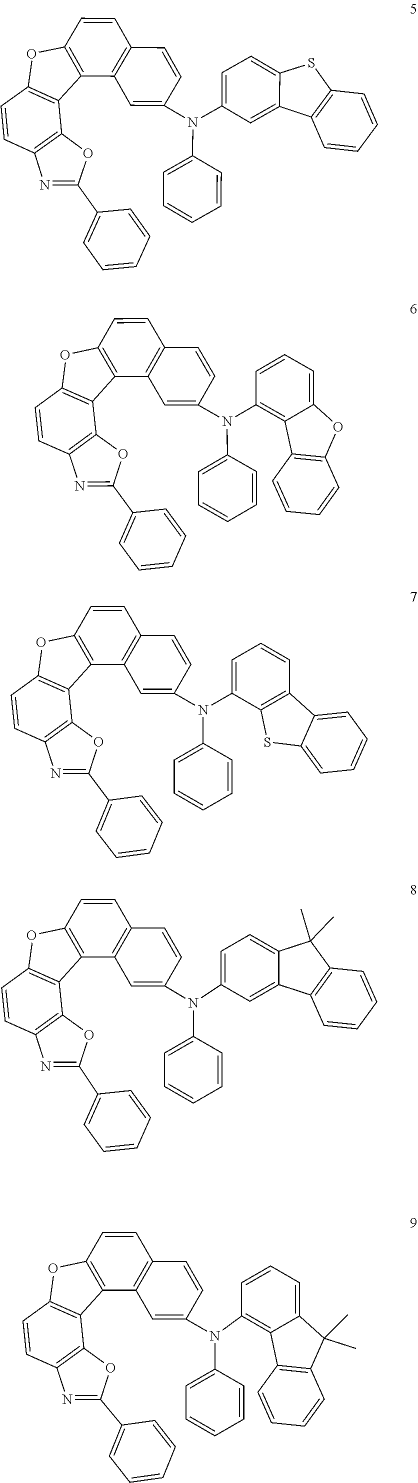

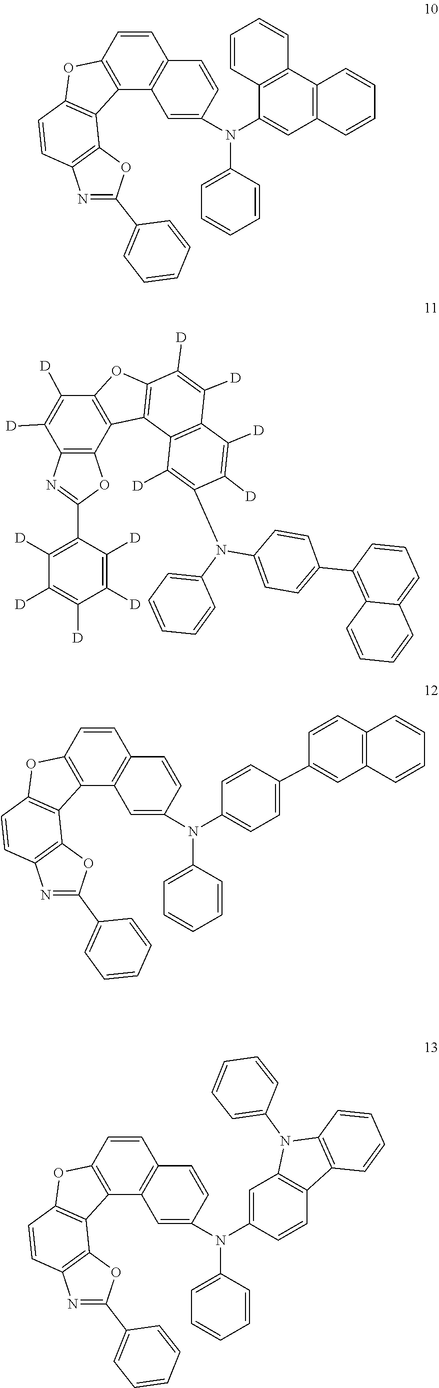 [SMILES: c1ccc(-c2nc3ccc4oc5ccc6ccc(N(c7ccccc7)c7cc8ccccc8c8ccccc78)cc6c5c4c3o2)cc1 ; [2H]c1c([2H])c([2H])c(-c2nc3c([2H])c([2H])c4oc5c([2H])c([2H])c6c([2H])c([2H])c(N(c7ccccc7)c7ccc(-c8cccc9ccccc89)cc7)c([2H])c6c5c4c3o2)c([2H])c1[2H] ; c1ccc(-c2nc3ccc4oc5ccc6ccc(N(c7ccccc7)c7ccc(-c8ccc9ccccc9c8)cc7)cc6c5c4c3o2)cc1 ; c1ccc(-c2nc3ccc4oc5ccc6ccc(N(c7ccccc7)c7ccc8c9ccccc9n(-c9ccccc9)c8c7)cc6c5c4c3o2)cc1]

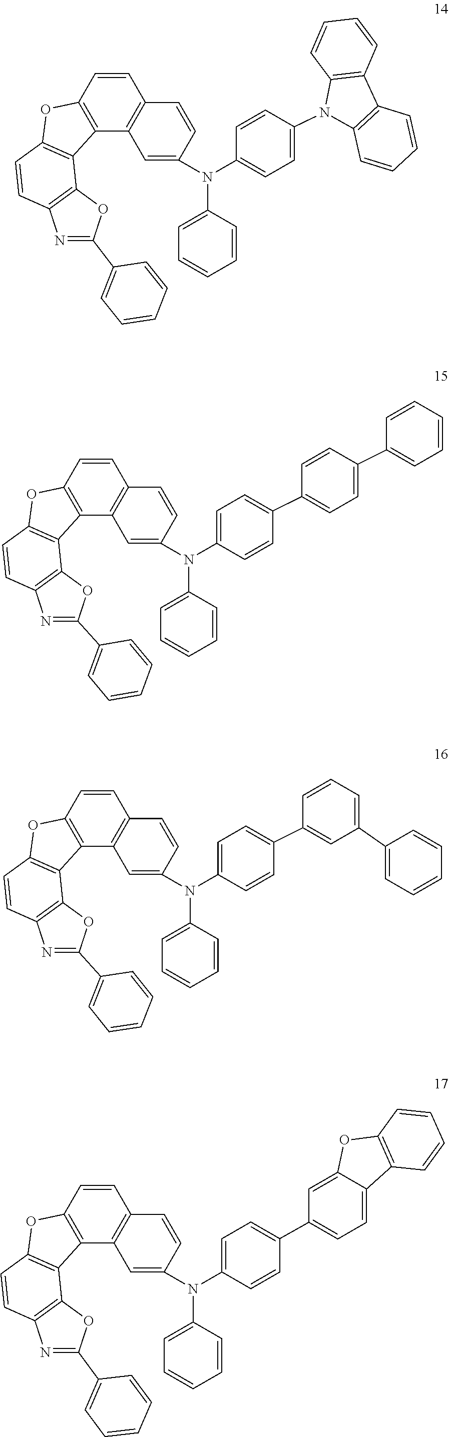 [SMILES: c1ccc(-c2nc3ccc4oc5ccc6ccc(N(c7ccccc7)c7ccc(-n8c9ccccc9c9ccccc98)cc7)cc6c5c4c3o2)cc1 ; c1ccc(-c2ccc(-c3ccc(N(c4ccccc4)c4ccc5ccc6oc7ccc8nc(-c9ccccc9)oc8c7c6c5c4)cc3)cc2)cc1 ; c1ccc(-c2cccc(-c3ccc(N(c4ccccc4)c4ccc5ccc6oc7ccc8nc(-c9ccccc9)oc8c7c6c5c4)cc3)c2)cc1 ; c1ccc(-c2nc3ccc4oc5ccc6ccc(N(c7ccccc7)c7ccc(-c8ccc9c(c8)oc8ccccc89)cc7)cc6c5c4c3o2)cc1]

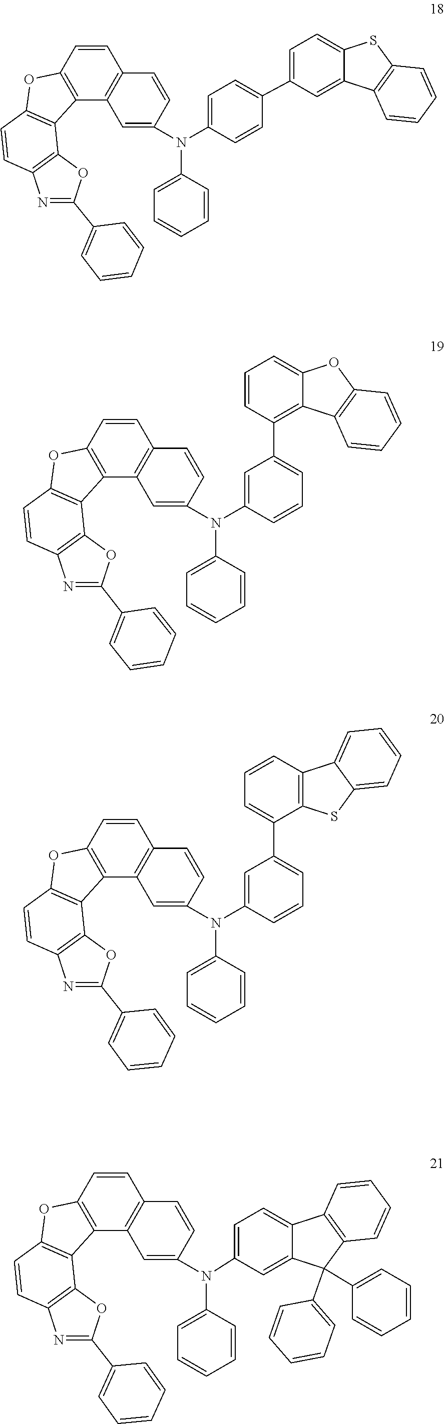 [SMILES: c1ccc(-c2nc3ccc4oc5ccc6ccc(N(c7ccccc7)c7ccc(-c8ccc9sc%10ccccc%10c9c8)cc7)cc6c5c4c3o2)cc1 ; c1ccc(-c2nc3ccc4oc5ccc6ccc(N(c7ccccc7)c7cccc(-c8cccc9oc%10ccccc%10c89)c7)cc6c5c4c3o2)cc1 ; c1ccc(-c2nc3ccc4oc5ccc6ccc(N(c7ccccc7)c7cccc(-c8cccc9c8sc8ccccc89)c7)cc6c5c4c3o2)cc1 ; c1ccc(-c2nc3ccc4oc5ccc6ccc(N(c7ccccc7)c7ccc8c(c7)C(c7ccccc7)(c7ccccc7)c7ccccc7-8)cc6c5c4c3o2)cc1]

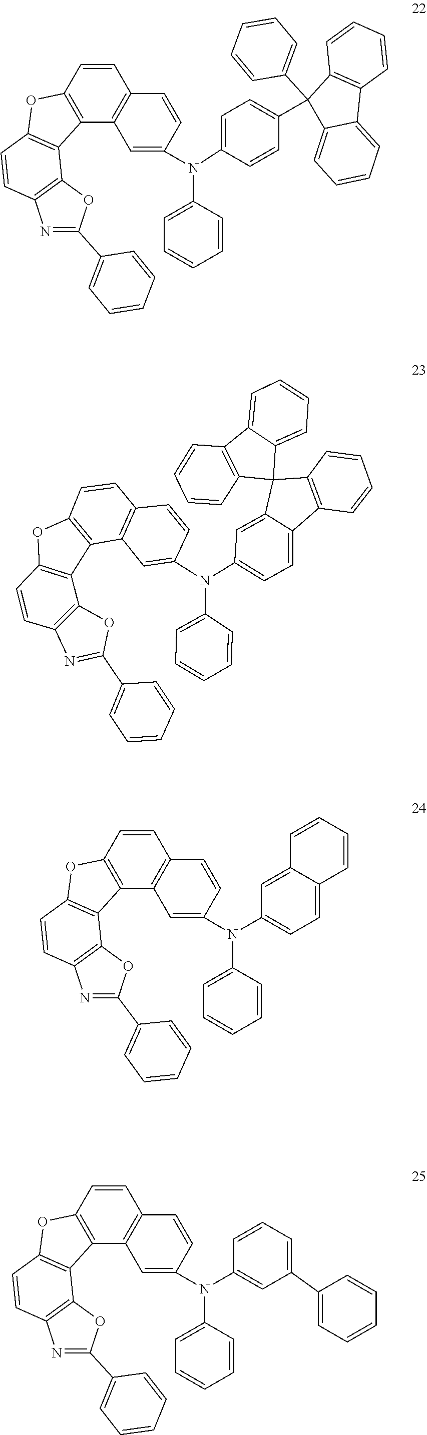 [SMILES: c1ccc(-c2nc3ccc4oc5ccc6ccc(N(c7ccccc7)c7ccc(C8(c9ccccc9)c9ccccc9-c9ccccc98)cc7)cc6c5c4c3o2)cc1 ; c1ccc(-c2nc3ccc4oc5ccc6ccc(N(c7ccccc7)c7ccc8c(c7)C7(c9ccccc9-c9ccccc97)c7ccccc7-8)cc6c5c4c3o2)cc1 ; c1ccc(-c2nc3ccc4oc5ccc6ccc(N(c7ccccc7)c7ccc8ccccc8c7)cc6c5c4c3o2)cc1 ; c1ccc(-c2cccc(N(c3ccccc3)c3ccc4ccc5oc6ccc7nc(-c8ccccc8)oc7c6c5c4c3)c2)cc1]

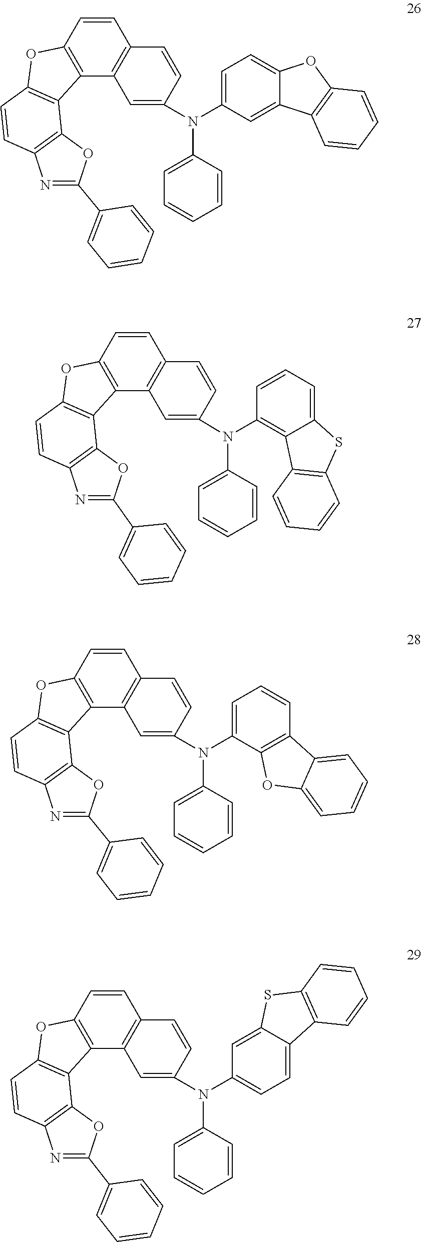 [SMILES: c1ccc(-c2nc3ccc4oc5ccc6ccc(N(c7ccccc7)c7ccc8oc9ccccc9c8c7)cc6c5c4c3o2)cc1 ; c1ccc(-c2nc3ccc4oc5ccc6ccc(N(c7ccccc7)c7cccc8sc9ccccc9c78)cc6c5c4c3o2)cc1 ; c1ccc(-c2nc3ccc4oc5ccc6ccc(N(c7ccccc7)c7cccc8c7oc7ccccc78)cc6c5c4c3o2)cc1 ; c1ccc(-c2nc3ccc4oc5ccc6ccc(N(c7ccccc7)c7ccc8c(c7)sc7ccccc78)cc6c5c4c3o2)cc1]

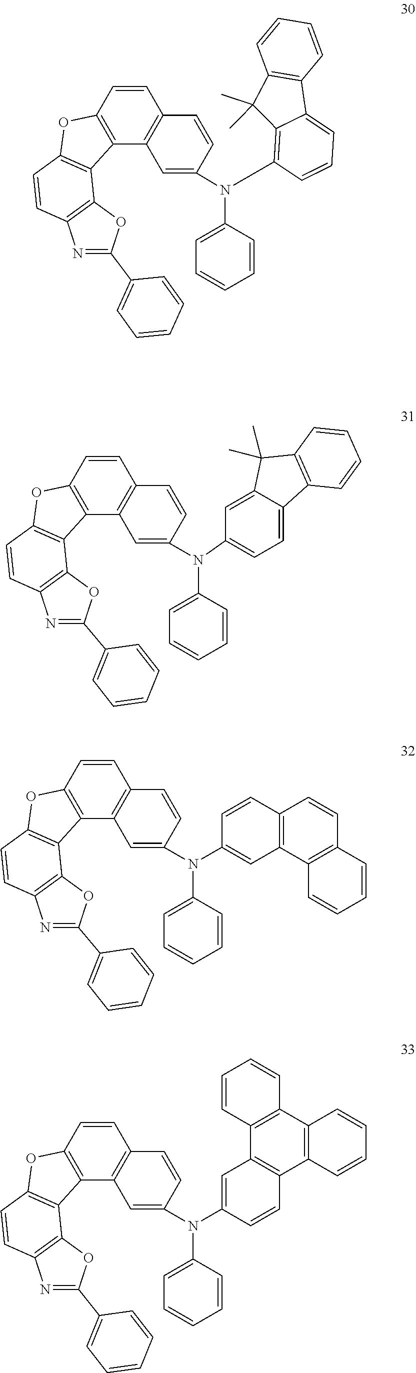 [SMILES: CC1(C)c2ccccc2-c2cccc(N(c3ccccc3)c3ccc4ccc5oc6ccc7nc(-c8ccccc8)oc7c6c5c4c3)c21 ; CC1(C)c2ccccc2-c2ccc(N(c3ccccc3)c3ccc4ccc5oc6ccc7nc(-c8ccccc8)oc7c6c5c4c3)cc21 ; c1ccc(-c2nc3ccc4oc5ccc6ccc(N(c7ccccc7)c7ccc8ccc9ccccc9c8c7)cc6c5c4c3o2)cc1 ; c1ccc(-c2nc3ccc4oc5ccc6ccc(N(c7ccccc7)c7ccc8c9ccccc9c9ccccc9c8c7)cc6c5c4c3o2)cc1]

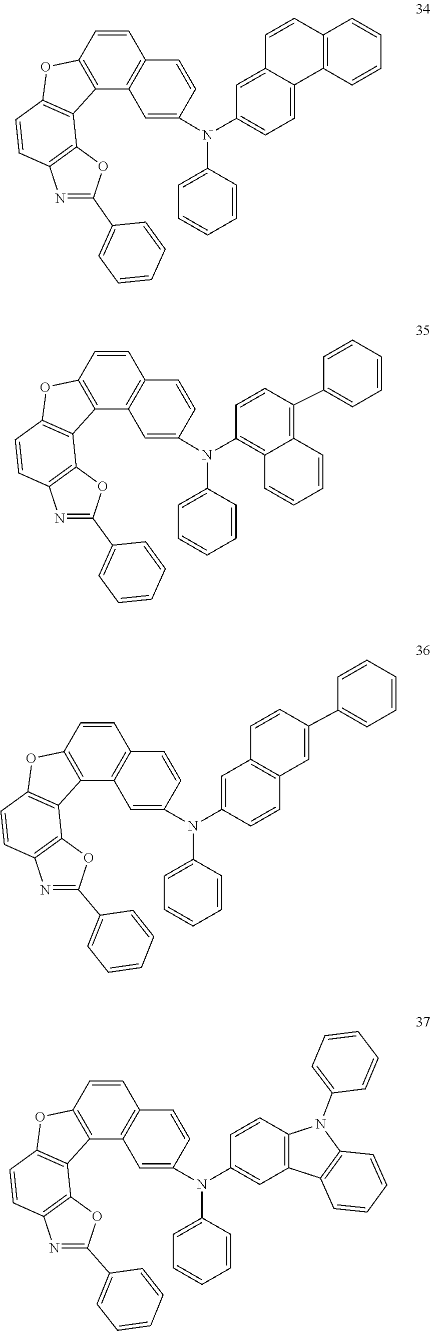 [SMILES: c1ccc(-c2nc3ccc4oc5ccc6ccc(N(c7ccccc7)c7ccc8c(ccc9ccccc98)c7)cc6c5c4c3o2)cc1 ; c1ccc(-c2nc3ccc4oc5ccc6ccc(N(c7ccccc7)c7ccc(-c8ccccc8)c8ccccc78)cc6c5c4c3o2)cc1 ; c1ccc(-c2ccc3cc(N(c4ccccc4)c4ccc5ccc6oc7ccc8nc(-c9ccccc9)oc8c7c6c5c4)ccc3c2)cc1 ; c1ccc(-c2nc3ccc4oc5ccc6ccc(N(c7ccccc7)c7ccc8c(c7)c7ccccc7n8-c7ccccc7)cc6c5c4c3o2)cc1]

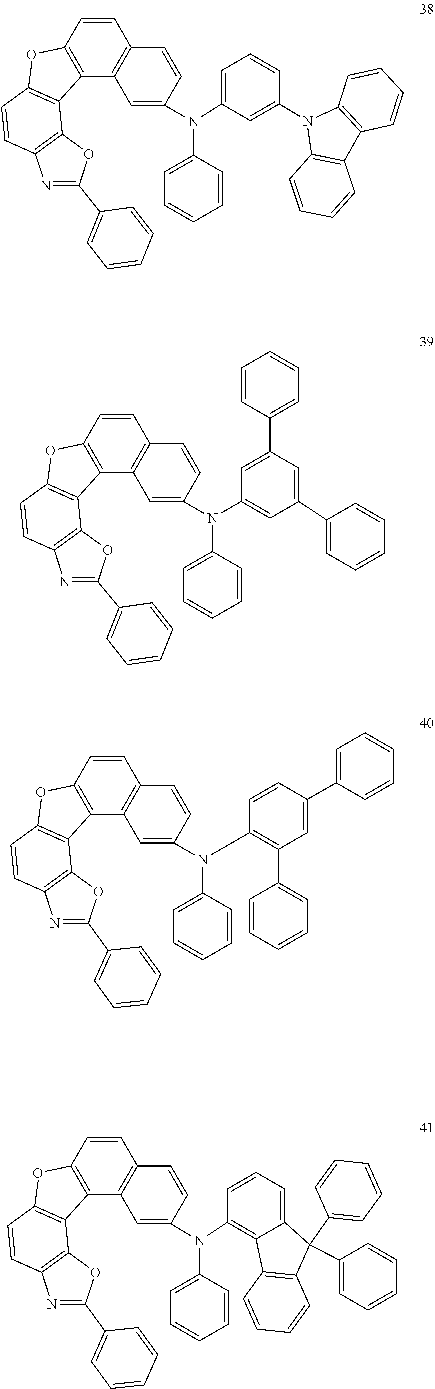 [SMILES: c1ccc(-c2nc3ccc4oc5ccc6ccc(N(c7ccccc7)c7cccc(-n8c9ccccc9c9ccccc98)c7)cc6c5c4c3o2)cc1 ; c1ccc(-c2cc(-c3ccccc3)cc(N(c3ccccc3)c3ccc4ccc5oc6ccc7nc(-c8ccccc8)oc7c6c5c4c3)c2)cc1 ; c1ccc(-c2ccc(N(c3ccccc3)c3ccc4ccc5oc6ccc7nc(-c8ccccc8)oc7c6c5c4c3)c(-c3ccccc3)c2)cc1 ; c1ccc(-c2nc3ccc4oc5ccc6ccc(N(c7ccccc7)c7cccc8c7-c7ccccc7C8(c7ccccc7)c7ccccc7)cc6c5c4c3o2)cc1]

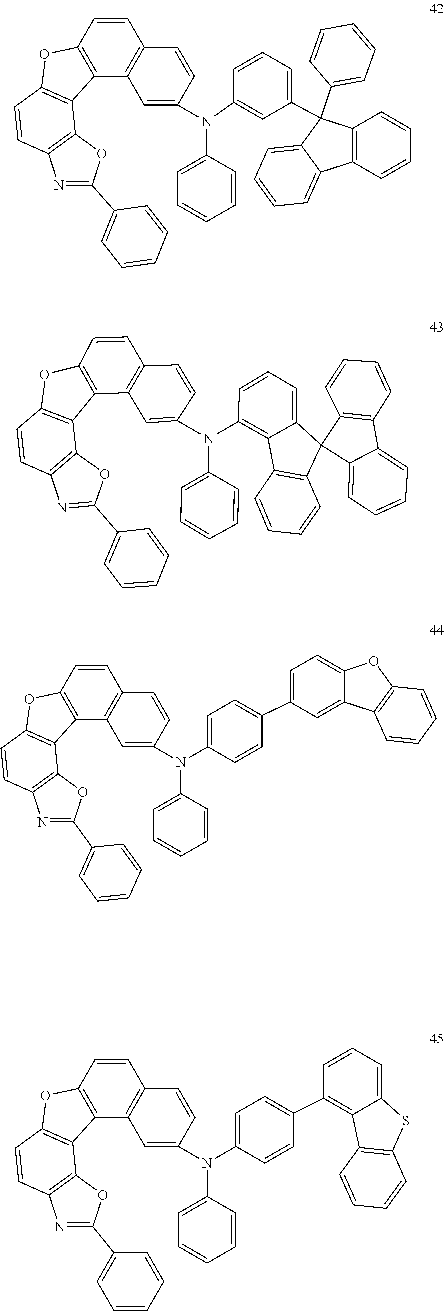 [SMILES: c1ccc(-c2nc3ccc4oc5ccc6ccc(N(c7ccccc7)c7cccc(C8(c9ccccc9)c9ccccc9-c9ccccc98)c7)cc6c5c4c3o2)cc1 ; c1ccc(-c2nc3ccc4oc5ccc6ccc(N(c7ccccc7)c7cccc8c7-c7ccccc7C87c8ccccc8-c8ccccc87)cc6c5c4c3o2)cc1 ; c1ccc(-c2nc3ccc4oc5ccc6ccc(N(c7ccccc7)c7ccc(-c8ccc9oc%10ccccc%10c9c8)cc7)cc6c5c4c3o2)cc1 ; c1ccc(-c2nc3ccc4oc5ccc6ccc(N(c7ccccc7)c7ccc(-c8cccc9sc%10ccccc%10c89)cc7)cc6c5c4c3o2)cc1]

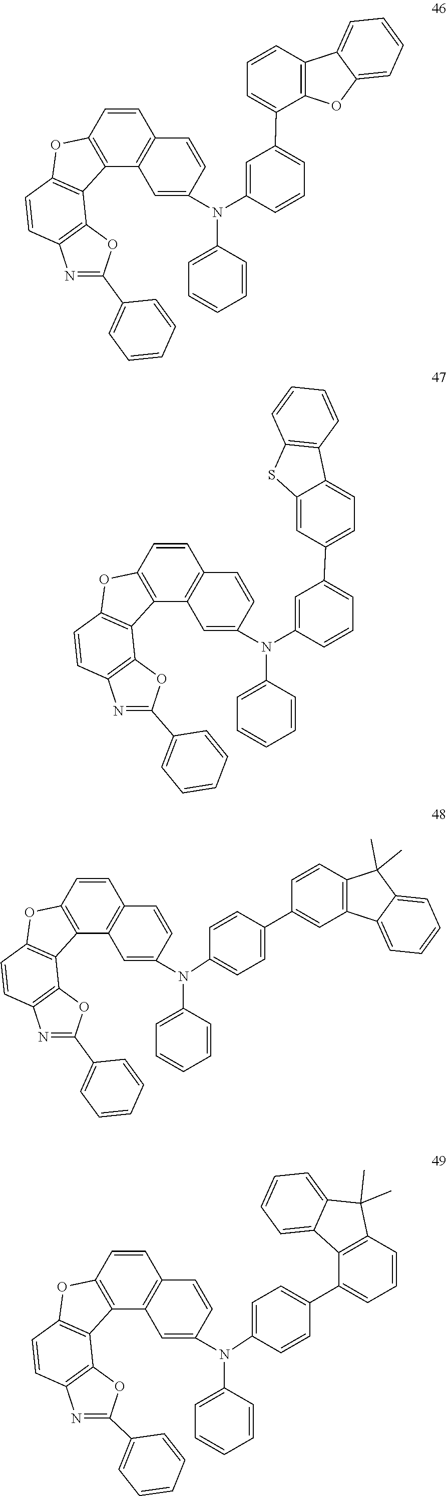 [SMILES: c1ccc(-c2nc3ccc4oc5ccc6ccc(N(c7ccccc7)c7cccc(-c8cccc9c8oc8ccccc89)c7)cc6c5c4c3o2)cc1 ; c1ccc(-c2nc3ccc4oc5ccc6ccc(N(c7ccccc7)c7cccc(-c8ccc9c(c8)sc8ccccc89)c7)cc6c5c4c3o2)cc1 ; CC1(C)c2ccccc2-c2cc(-c3ccc(N(c4ccccc4)c4ccc5ccc6oc7ccc8nc(-c9ccccc9)oc8c7c6c5c4)cc3)ccc21 ; CC1(C)c2ccccc2-c2c(-c3ccc(N(c4ccccc4)c4ccc5ccc6oc7ccc8nc(-c9ccccc9)oc8c7c6c5c4)cc3)cccc21]

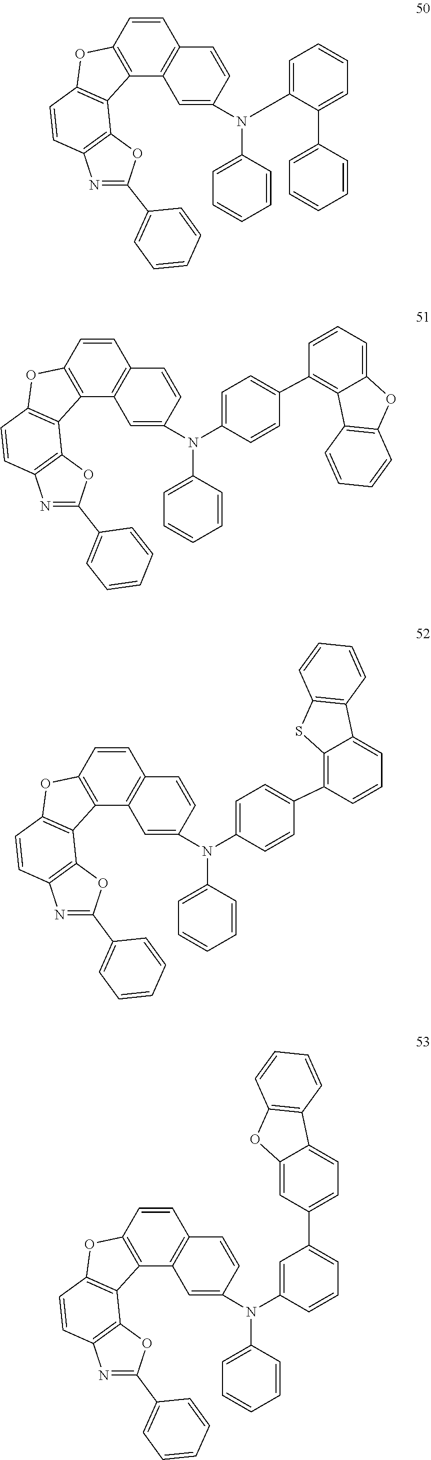 [SMILES: c1ccc(-c2nc3ccc4oc5ccc6ccc(N(c7ccccc7)c7ccccc7-c7ccccc7)cc6c5c4c3o2)cc1 ; c1ccc(-c2nc3ccc4oc5ccc6ccc(N(c7ccccc7)c7ccc(-c8cccc9oc%10ccccc%10c89)cc7)cc6c5c4c3o2)cc1 ; c1ccc(-c2nc3ccc4oc5ccc6ccc(N(c7ccccc7)c7ccc(-c8cccc9c8sc8ccccc89)cc7)cc6c5c4c3o2)cc1 ; c1ccc(-c2nc3ccc4oc5ccc6ccc(N(c7ccccc7)c7cccc(-c8ccc9c(c8)oc8ccccc89)c7)cc6c5c4c3o2)cc1]

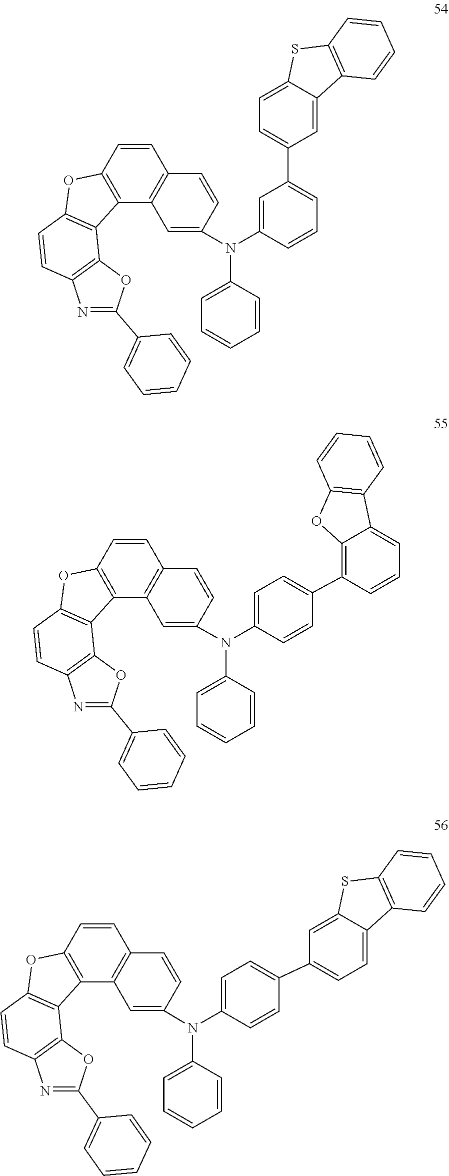 [SMILES: c1ccc(-c2nc3ccc4oc5ccc6ccc(N(c7ccccc7)c7cccc(-c8ccc9sc%10ccccc%10c9c8)c7)cc6c5c4c3o2)cc1 ; c1ccc(-c2nc3ccc4oc5ccc6ccc(N(c7ccccc7)c7ccc(-c8cccc9c8oc8ccccc89)cc7)cc6c5c4c3o2)cc1 ; c1ccc(-c2nc3ccc4oc5ccc6ccc(N(c7ccccc7)c7ccc(-c8ccc9c(c8)sc8ccccc89)cc7)cc6c5c4c3o2)cc1]

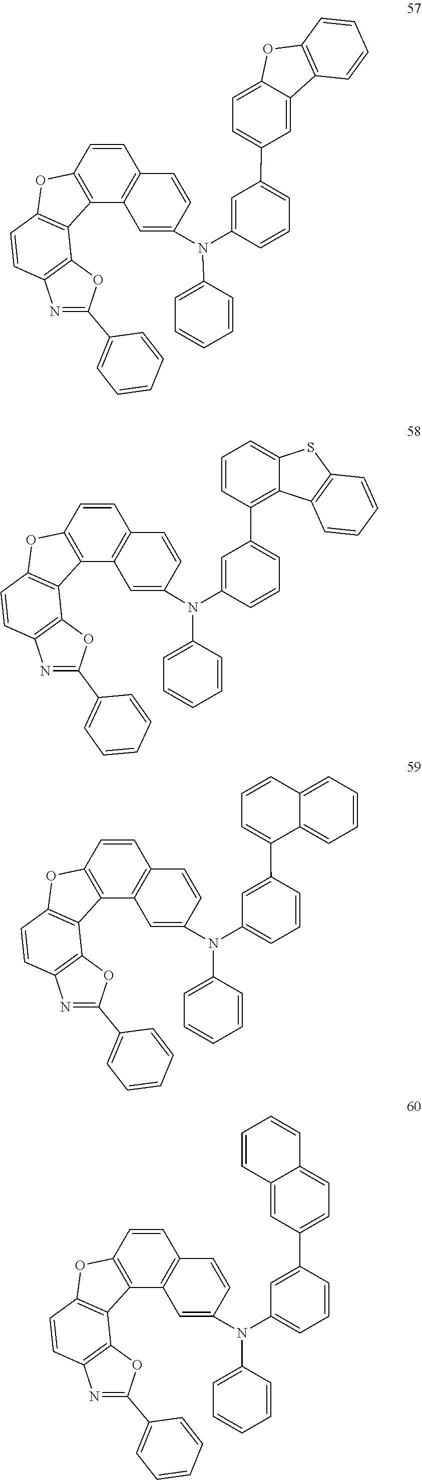 [SMILES: c1ccc(-c2nc3ccc4oc5ccc6ccc(N(c7ccccc7)c7cccc(-c8ccc9oc%10ccccc%10c9c8)c7)cc6c5c4c3o2)cc1 ; c1ccc(-c2nc3ccc4oc5ccc6ccc(N(c7ccccc7)c7cccc(-c8cccc9sc%10ccccc%10c89)c7)cc6c5c4c3o2)cc1 ; c1ccc(-c2nc3ccc4oc5ccc6ccc(N(c7ccccc7)c7cccc(-c8cccc9ccccc89)c7)cc6c5c4c3o2)cc1 ; c1ccc(-c2nc3ccc4oc5ccc6ccc(N(c7ccccc7)c7cccc(-c8ccc9ccccc9c8)c7)cc6c5c4c3o2)cc1]

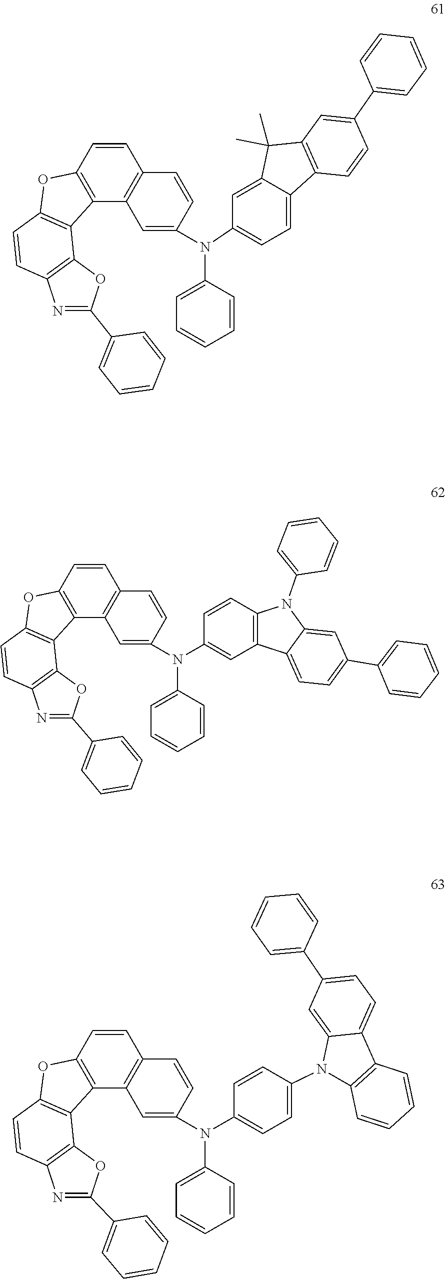 [SMILES: CC1(C)c2cc(-c3ccccc3)ccc2-c2ccc(N(c3ccccc3)c3ccc4ccc5oc6ccc7nc(-c8ccccc8)oc7c6c5c4c3)cc21 ; c1ccc(-c2ccc3c4cc(N(c5ccccc5)c5ccc6ccc7oc8ccc9nc(-c%10ccccc%10)oc9c8c7c6c5)ccc4n(-c4ccccc4)c3c2)cc1 ; c1ccc(-c2ccc3c4ccccc4n(-c4ccc(N(c5ccccc5)c5ccc6ccc7oc8ccc9nc(-c%10ccccc%10)oc9c8c7c6c5)cc4)c3c2)cc1]

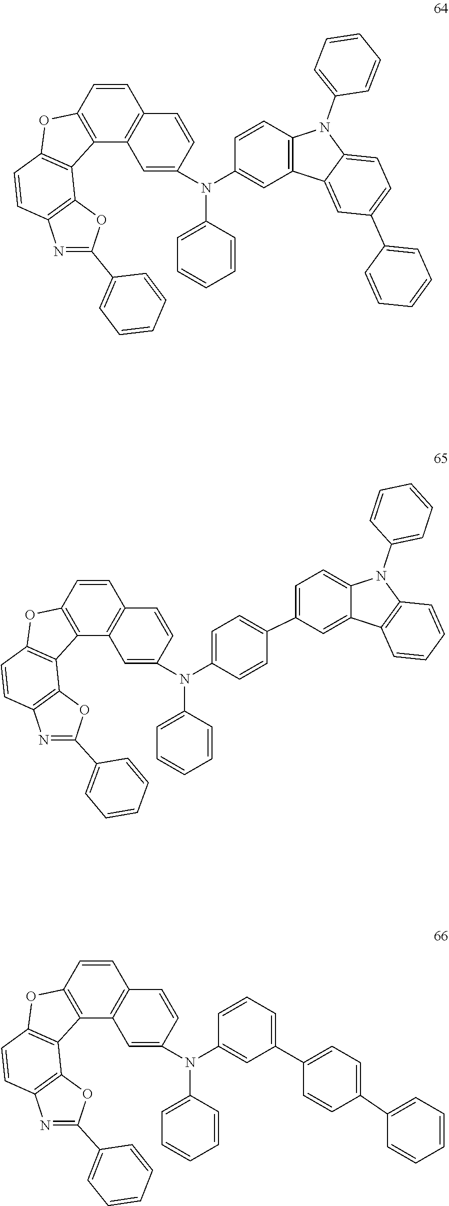 [SMILES: c1ccc(-c2ccc3c(c2)c2cc(N(c4ccccc4)c4ccc5ccc6oc7ccc8nc(-c9ccccc9)oc8c7c6c5c4)ccc2n3-c2ccccc2)cc1 ; c1ccc(-c2nc3ccc4oc5ccc6ccc(N(c7ccccc7)c7ccc(-c8ccc9c(c8)c8ccccc8n9-c8ccccc8)cc7)cc6c5c4c3o2)cc1 ; c1ccc(-c2ccc(-c3cccc(N(c4ccccc4)c4ccc5ccc6oc7ccc8nc(-c9ccccc9)oc8c7c6c5c4)c3)cc2)cc1]

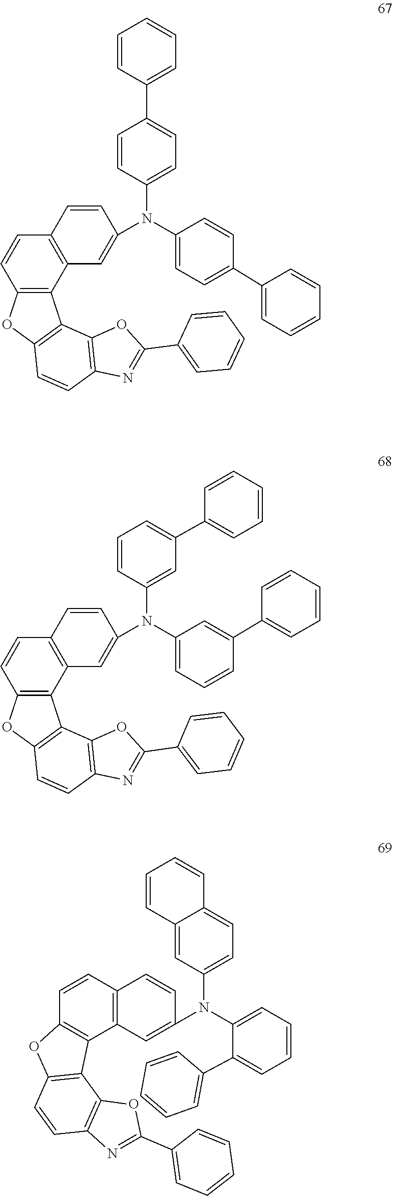 [SMILES: c1ccc(-c2ccc(N(c3ccc(-c4ccccc4)cc3)c3ccc4ccc5oc6ccc7nc(-c8ccccc8)oc7c6c5c4c3)cc2)cc1 ; c1ccc(-c2cccc(N(c3cccc(-c4ccccc4)c3)c3ccc4ccc5oc6ccc7nc(-c8ccccc8)oc7c6c5c4c3)c2)cc1 ; c1ccc(-c2nc3ccc4oc5ccc6ccc(N(c7ccc8ccccc8c7)c7ccccc7-c7ccccc7)cc6c5c4c3o2)cc1]

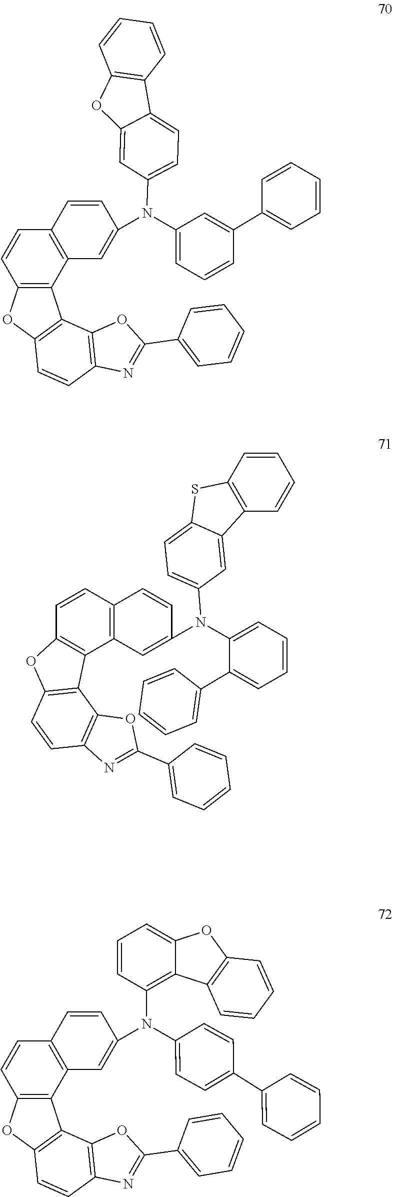 [SMILES: c1ccc(-c2cccc(N(c3ccc4c(c3)oc3ccccc34)c3ccc4ccc5oc6ccc7nc(-c8ccccc8)oc7c6c5c4c3)c2)cc1 ; c1ccc(-c2nc3ccc4oc5ccc6ccc(N(c7ccc8sc9ccccc9c8c7)c7ccccc7-c7ccccc7)cc6c5c4c3o2)cc1 ; c1ccc(-c2ccc(N(c3ccc4ccc5oc6ccc7nc(-c8ccccc8)oc7c6c5c4c3)c3cccc4oc5ccccc5c34)cc2)cc1]

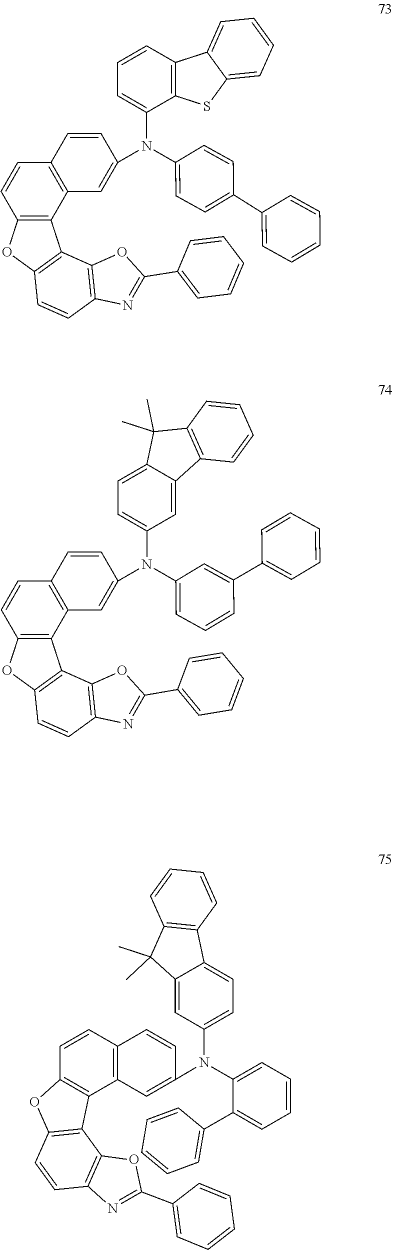 [SMILES: c1ccc(-c2ccc(N(c3ccc4ccc5oc6ccc7nc(-c8ccccc8)oc7c6c5c4c3)c3cccc4c3sc3ccccc34)cc2)cc1 ; CC1(C)c2ccccc2-c2cc(N(c3cccc(-c4ccccc4)c3)c3ccc4ccc5oc6ccc7nc(-c8ccccc8)oc7c6c5c4c3)ccc21 ; CC1(C)c2ccccc2-c2ccc(N(c3ccc4ccc5oc6ccc7nc(-c8ccccc8)oc7c6c5c4c3)c3ccccc3-c3ccccc3)cc21]

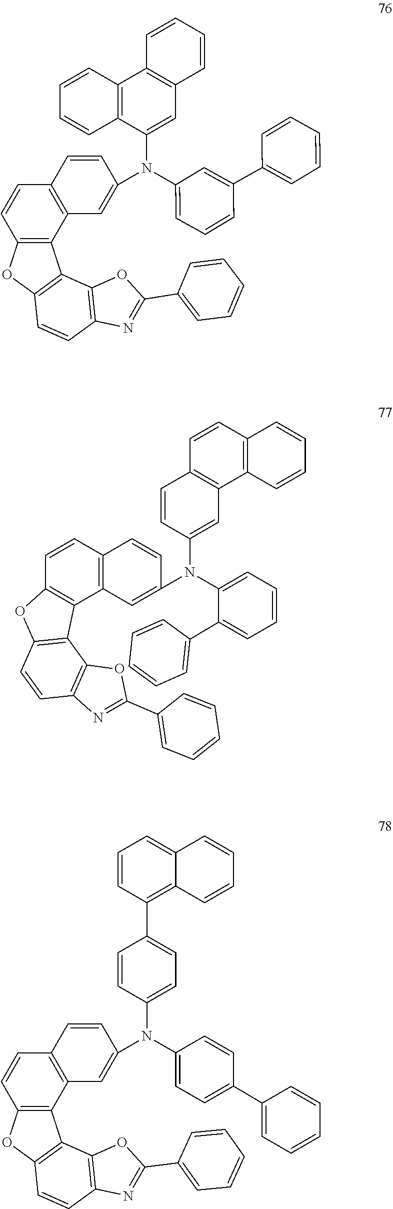 [SMILES: c1ccc(-c2cccc(N(c3ccc4ccc5oc6ccc7nc(-c8ccccc8)oc7c6c5c4c3)c3cc4ccccc4c4ccccc34)c2)cc1 ; c1ccc(-c2nc3ccc4oc5ccc6ccc(N(c7ccc8ccc9ccccc9c8c7)c7ccccc7-c7ccccc7)cc6c5c4c3o2)cc1 ; c1ccc(-c2ccc(N(c3ccc(-c4cccc5ccccc45)cc3)c3ccc4ccc5oc6ccc7nc(-c8ccccc8)oc7c6c5c4c3)cc2)cc1]

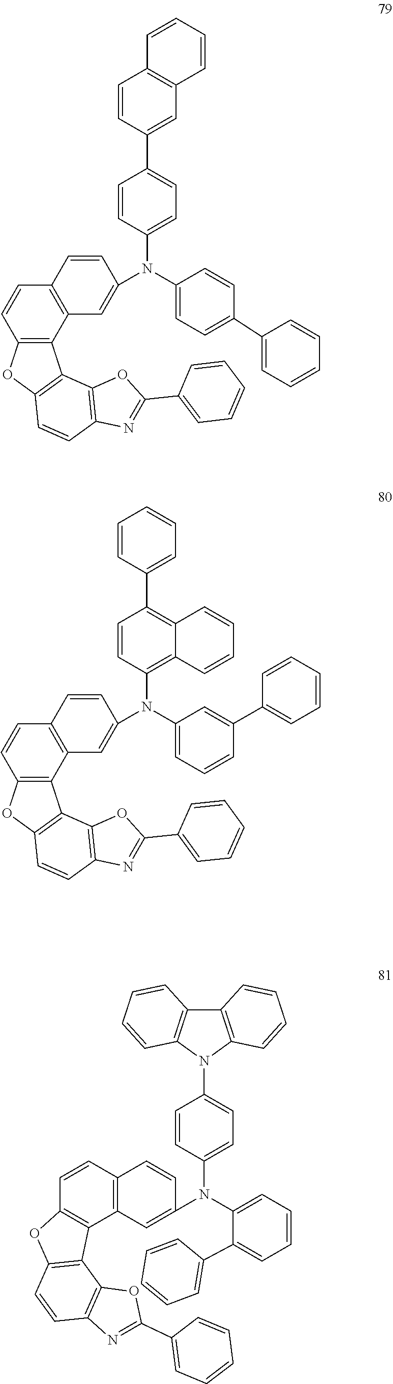 [SMILES: c1ccc(-c2ccc(N(c3ccc(-c4ccc5ccccc5c4)cc3)c3ccc4ccc5oc6ccc7nc(-c8ccccc8)oc7c6c5c4c3)cc2)cc1 ; c1ccc(-c2cccc(N(c3ccc4ccc5oc6ccc7nc(-c8ccccc8)oc7c6c5c4c3)c3ccc(-c4ccccc4)c4ccccc34)c2)cc1 ; c1ccc(-c2nc3ccc4oc5ccc6ccc(N(c7ccc(-n8c9ccccc9c9ccccc98)cc7)c7ccccc7-c7ccccc7)cc6c5c4c3o2)cc1]

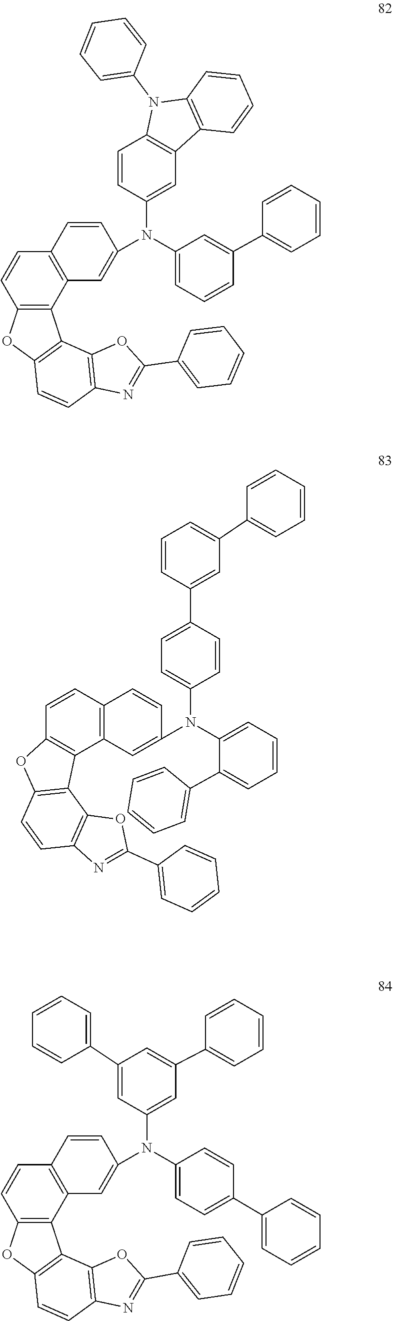 [SMILES: c1ccc(-c2cccc(N(c3ccc4ccc5oc6ccc7nc(-c8ccccc8)oc7c6c5c4c3)c3ccc4c(c3)c3ccccc3n4-c3ccccc3)c2)cc1 ; c1ccc(-c2cccc(-c3ccc(N(c4ccc5ccc6oc7ccc8nc(-c9ccccc9)oc8c7c6c5c4)c4ccccc4-c4ccccc4)cc3)c2)cc1 ; c1ccc(-c2ccc(N(c3cc(-c4ccccc4)cc(-c4ccccc4)c3)c3ccc4ccc5oc6ccc7nc(-c8ccccc8)oc7c6c5c4c3)cc2)cc1]

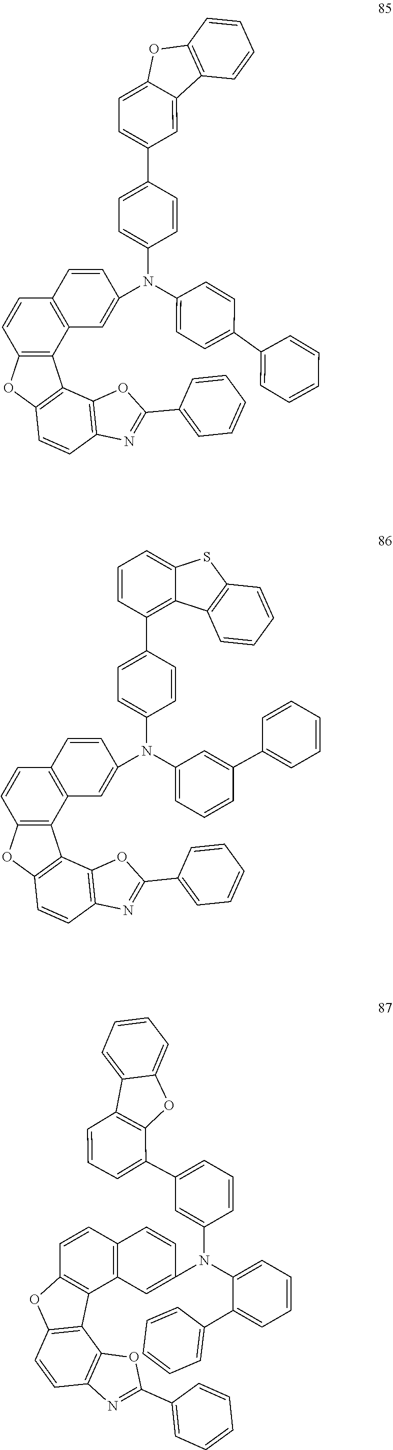 [SMILES: c1ccc(-c2ccc(N(c3ccc(-c4ccc5oc6ccccc6c5c4)cc3)c3ccc4ccc5oc6ccc7nc(-c8ccccc8)oc7c6c5c4c3)cc2)cc1 ; c1ccc(-c2cccc(N(c3ccc(-c4cccc5sc6ccccc6c45)cc3)c3ccc4ccc5oc6ccc7nc(-c8ccccc8)oc7c6c5c4c3)c2)cc1 ; c1ccc(-c2nc3ccc4oc5ccc6ccc(N(c7cccc(-c8cccc9c8oc8ccccc89)c7)c7ccccc7-c7ccccc7)cc6c5c4c3o2)cc1]

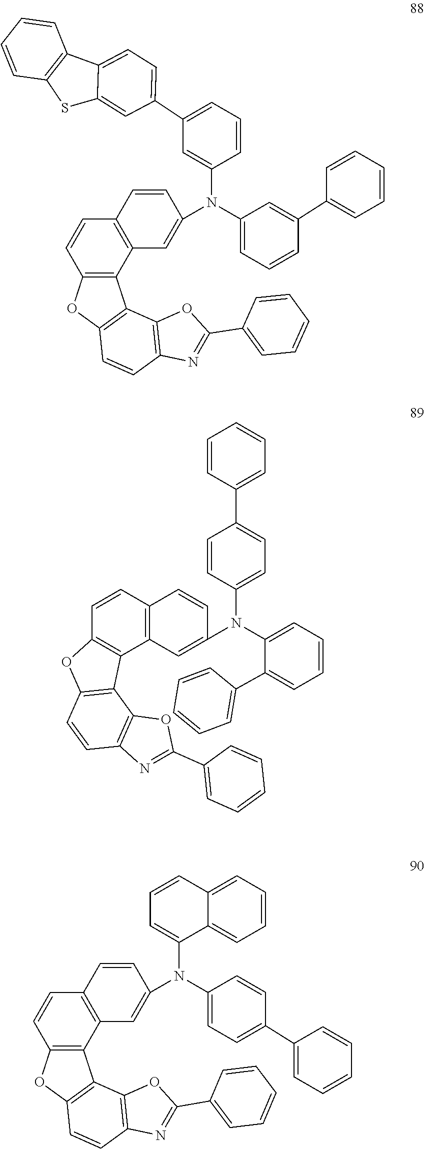 [SMILES: c1ccc(-c2cccc(N(c3cccc(-c4ccc5c(c4)sc4ccccc45)c3)c3ccc4ccc5oc6ccc7nc(-c8ccccc8)oc7c6c5c4c3)c2)cc1 ; c1ccc(-c2ccc(N(c3ccc4ccc5oc6ccc7nc(-c8ccccc8)oc7c6c5c4c3)c3ccccc3-c3ccccc3)cc2)cc1 ; c1ccc(-c2ccc(N(c3ccc4ccc5oc6ccc7nc(-c8ccccc8)oc7c6c5c4c3)c3cccc4ccccc34)cc2)cc1]

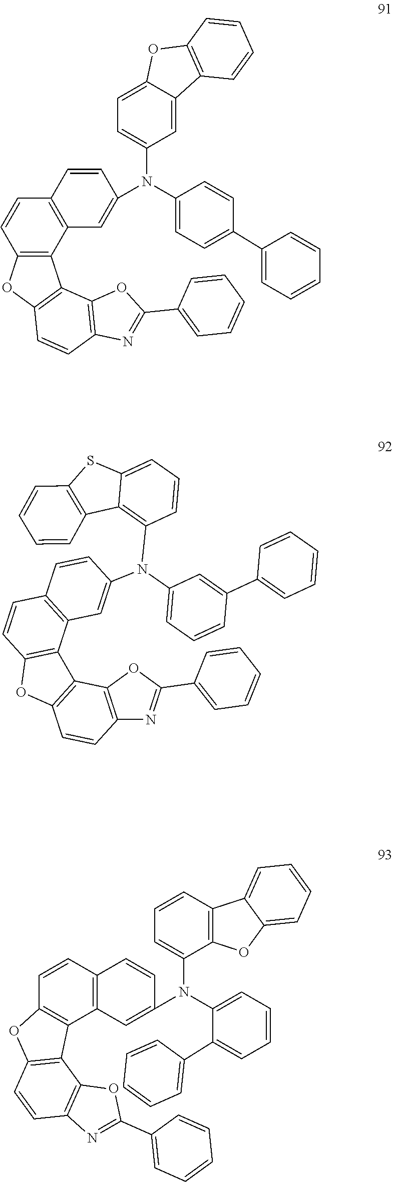 [SMILES: c1ccc(-c2ccc(N(c3ccc4oc5ccccc5c4c3)c3ccc4ccc5oc6ccc7nc(-c8ccccc8)oc7c6c5c4c3)cc2)cc1 ; c1ccc(-c2cccc(N(c3ccc4ccc5oc6ccc7nc(-c8ccccc8)oc7c6c5c4c3)c3cccc4sc5ccccc5c34)c2)cc1 ; c1ccc(-c2nc3ccc4oc5ccc6ccc(N(c7ccccc7-c7ccccc7)c7cccc8c7oc7ccccc78)cc6c5c4c3o2)cc1]

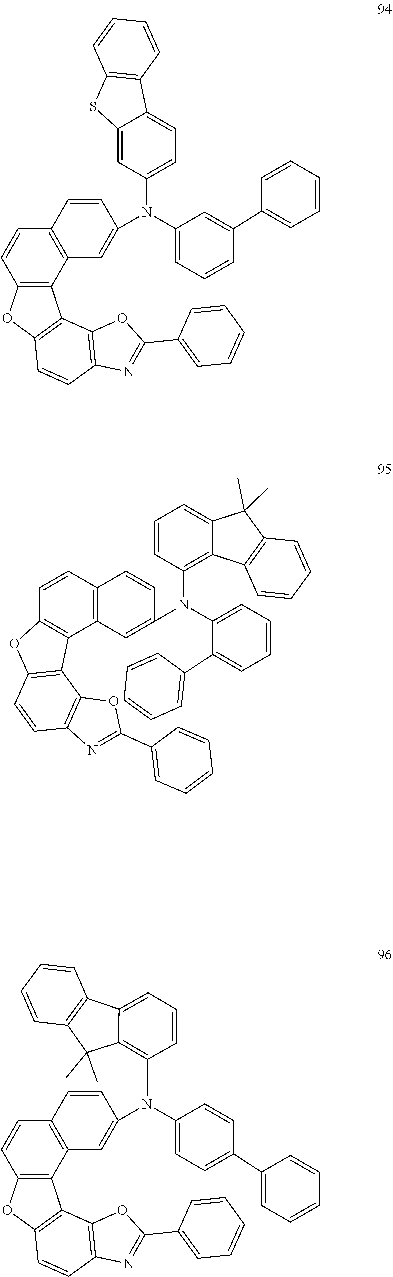 [SMILES: c1ccc(-c2cccc(N(c3ccc4c(c3)sc3ccccc34)c3ccc4ccc5oc6ccc7nc(-c8ccccc8)oc7c6c5c4c3)c2)cc1 ; CC1(C)c2ccccc2-c2c(N(c3ccc4ccc5oc6ccc7nc(-c8ccccc8)oc7c6c5c4c3)c3ccccc3-c3ccccc3)cccc21 ; CC1(C)c2ccccc2-c2cccc(N(c3ccc(-c4ccccc4)cc3)c3ccc4ccc5oc6ccc7nc(-c8ccccc8)oc7c6c5c4c3)c21]

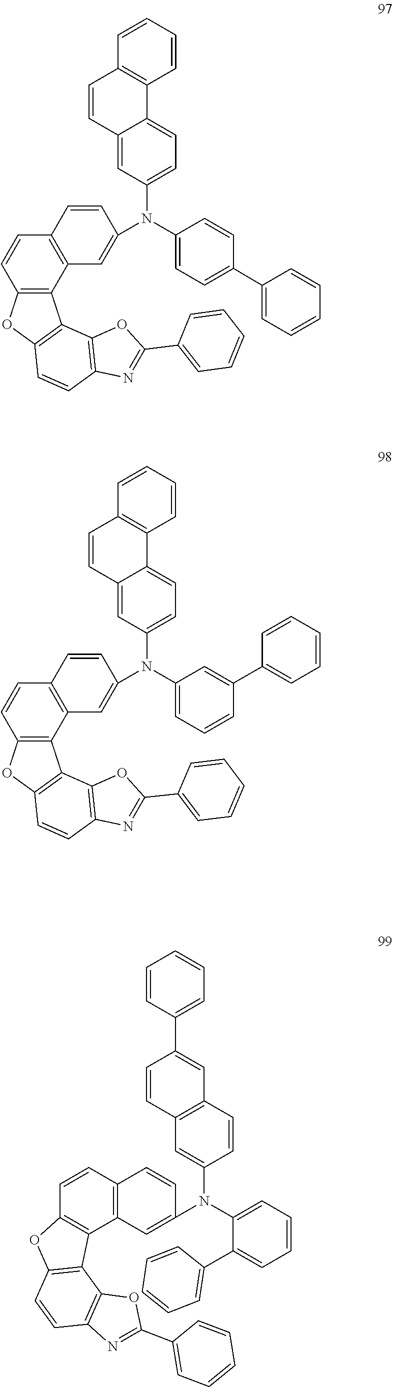 [SMILES: c1ccc(-c2ccc(N(c3ccc4c(ccc5ccccc54)c3)c3ccc4ccc5oc6ccc7nc(-c8ccccc8)oc7c6c5c4c3)cc2)cc1 ; c1ccc(-c2cccc(N(c3ccc4c(ccc5ccccc54)c3)c3ccc4ccc5oc6ccc7nc(-c8ccccc8)oc7c6c5c4c3)c2)cc1 ; c1ccc(-c2ccc3cc(N(c4ccc5ccc6oc7ccc8nc(-c9ccccc9)oc8c7c6c5c4)c4ccccc4-c4ccccc4)ccc3c2)cc1]

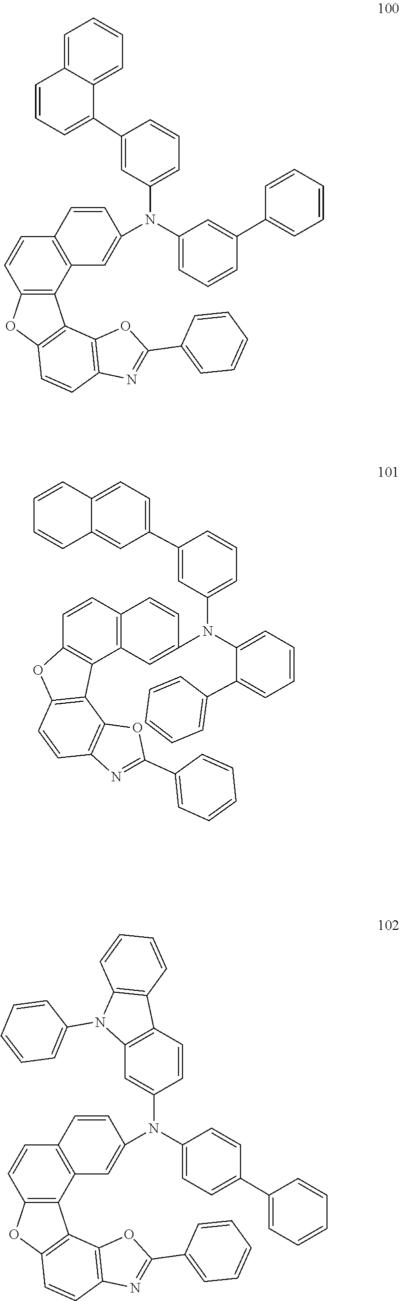 [SMILES: c1ccc(-c2cccc(N(c3cccc(-c4cccc5ccccc45)c3)c3ccc4ccc5oc6ccc7nc(-c8ccccc8)oc7c6c5c4c3)c2)cc1 ; c1ccc(-c2nc3ccc4oc5ccc6ccc(N(c7cccc(-c8ccc9ccccc9c8)c7)c7ccccc7-c7ccccc7)cc6c5c4c3o2)cc1 ; c1ccc(-c2ccc(N(c3ccc4ccc5oc6ccc7nc(-c8ccccc8)oc7c6c5c4c3)c3ccc4c5ccccc5n(-c5ccccc5)c4c3)cc2)cc1]

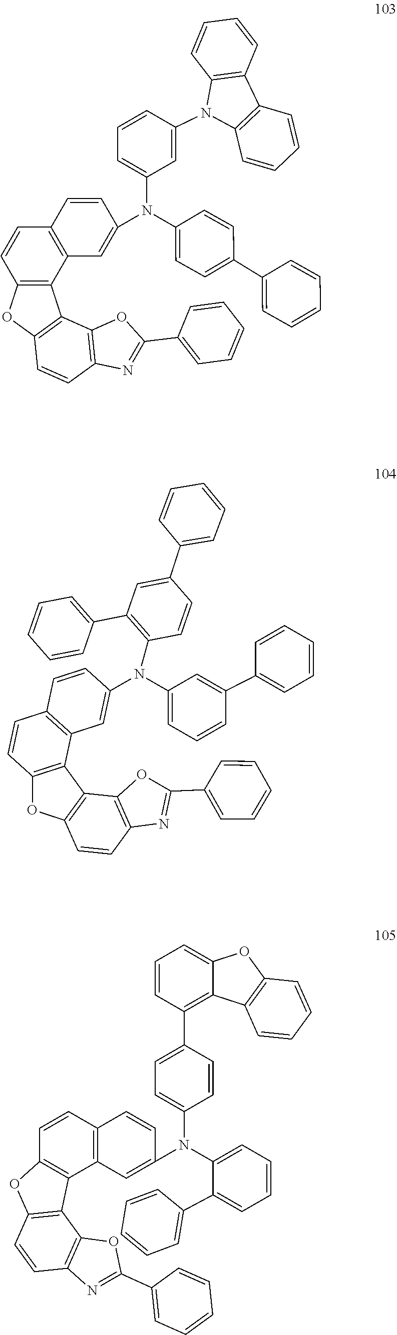

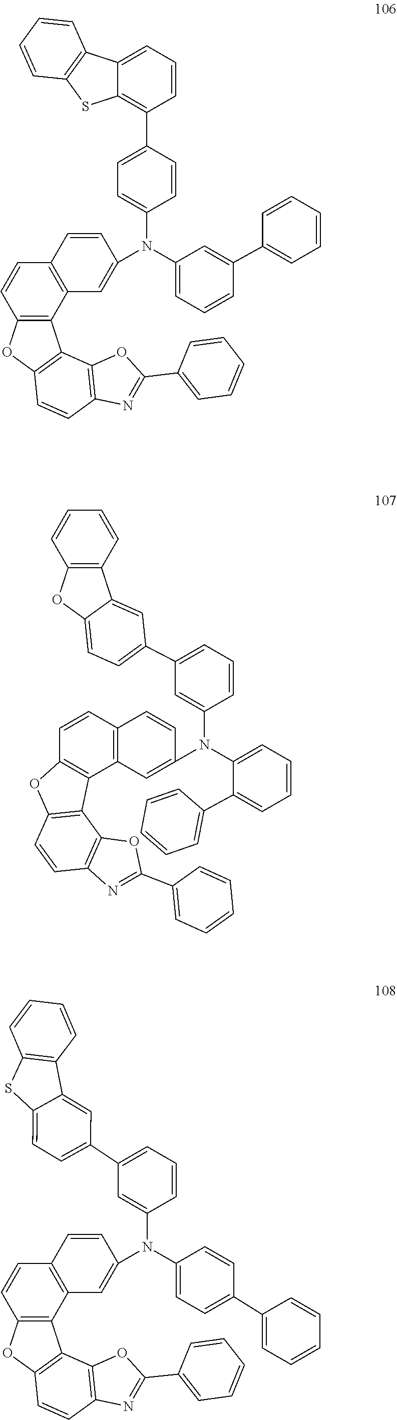 [SMILES: c1ccc(-c2cccc(N(c3ccc(-c4cccc5c4sc4ccccc45)cc3)c3ccc4ccc5oc6ccc7nc(-c8ccccc8)oc7c6c5c4c3)c2)cc1 ; c1ccc(-c2nc3ccc4oc5ccc6ccc(N(c7cccc(-c8ccc9oc%10ccccc%10c9c8)c7)c7ccccc7-c7ccccc7)cc6c5c4c3o2)cc1 ; c1ccc(-c2ccc(N(c3cccc(-c4ccc5sc6ccccc6c5c4)c3)c3ccc4ccc5oc6ccc7nc(-c8ccccc8)oc7c6c5c4c3)cc2)cc1]

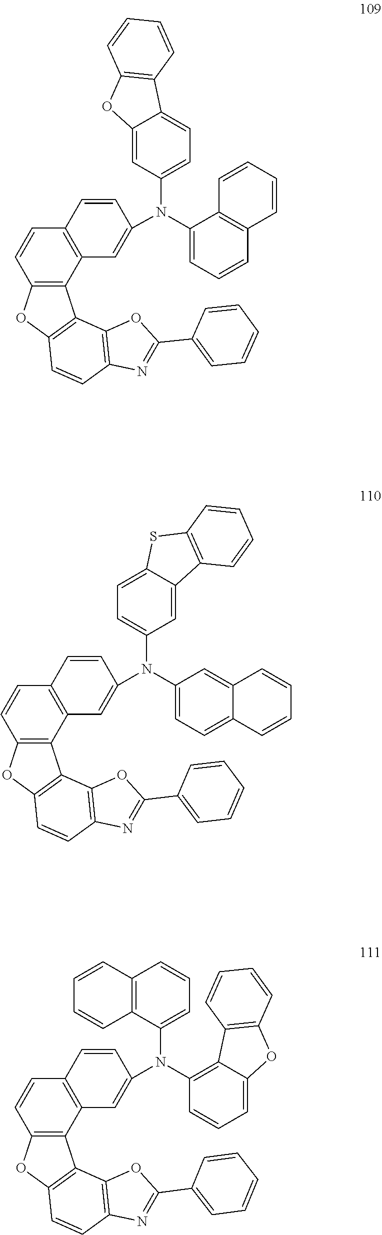 [SMILES: c1ccc(-c2nc3ccc4oc5ccc6ccc(N(c7ccc8c(c7)oc7ccccc78)c7cccc8ccccc78)cc6c5c4c3o2)cc1 ; c1ccc(-c2nc3ccc4oc5ccc6ccc(N(c7ccc8ccccc8c7)c7ccc8sc9ccccc9c8c7)cc6c5c4c3o2)cc1 ; c1ccc(-c2nc3ccc4oc5ccc6ccc(N(c7cccc8ccccc78)c7cccc8oc9ccccc9c78)cc6c5c4c3o2)cc1]

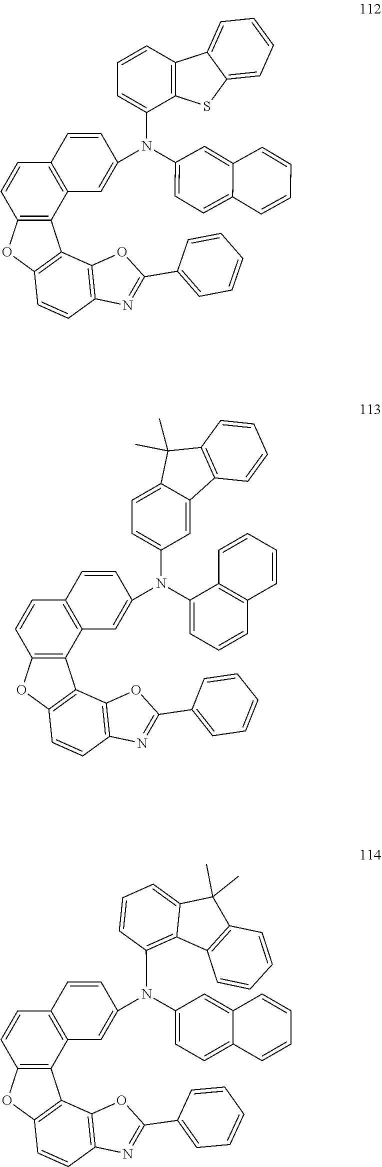 [SMILES: c1ccc(-c2nc3ccc4oc5ccc6ccc(N(c7ccc8ccccc8c7)c7cccc8c7sc7ccccc78)cc6c5c4c3o2)cc1 ; CC1(C)c2ccccc2-c2cc(N(c3ccc4ccc5oc6ccc7nc(-c8ccccc8)oc7c6c5c4c3)c3cccc4ccccc34)ccc21 ; CC1(C)c2ccccc2-c2c(N(c3ccc4ccccc4c3)c3ccc4ccc5oc6ccc7nc(-c8ccccc8)oc7c6c5c4c3)cccc21]

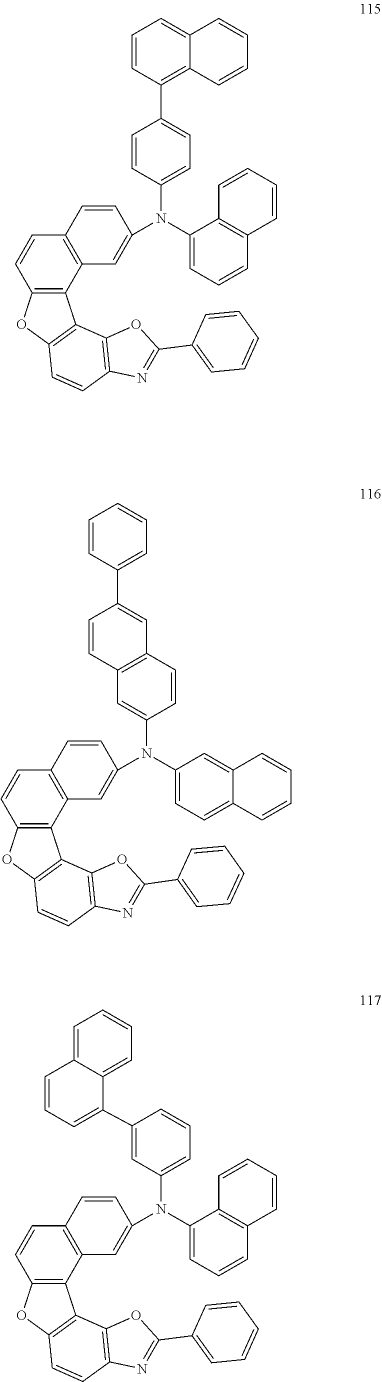 [SMILES: c1ccc(-c2nc3ccc4oc5ccc6ccc(N(c7ccc(-c8cccc9ccccc89)cc7)c7cccc8ccccc78)cc6c5c4c3o2)cc1 ; c1ccc(-c2ccc3cc(N(c4ccc5ccccc5c4)c4ccc5ccc6oc7ccc8nc(-c9ccccc9)oc8c7c6c5c4)ccc3c2)cc1 ; c1ccc(-c2nc3ccc4oc5ccc6ccc(N(c7cccc(-c8cccc9ccccc89)c7)c7cccc8ccccc78)cc6c5c4c3o2)cc1]

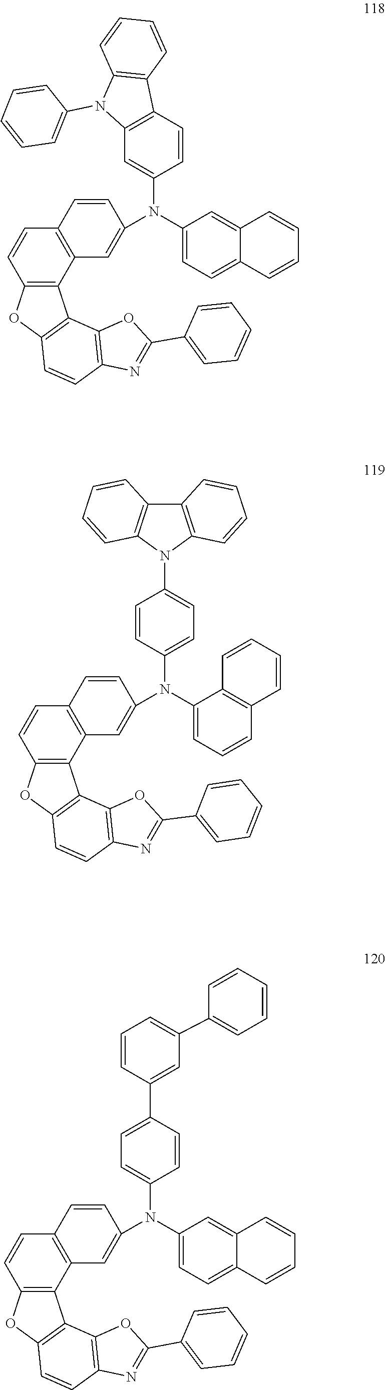 [SMILES: c1ccc(-c2nc3ccc4oc5ccc6ccc(N(c7ccc8ccccc8c7)c7ccc8c9ccccc9n(-c9ccccc9)c8c7)cc6c5c4c3o2)cc1 ; c1ccc(-c2nc3ccc4oc5ccc6ccc(N(c7ccc(-n8c9ccccc9c9ccccc98)cc7)c7cccc8ccccc78)cc6c5c4c3o2)cc1 ; c1ccc(-c2cccc(-c3ccc(N(c4ccc5ccccc5c4)c4ccc5ccc6oc7ccc8nc(-c9ccccc9)oc8c7c6c5c4)cc3)c2)cc1]

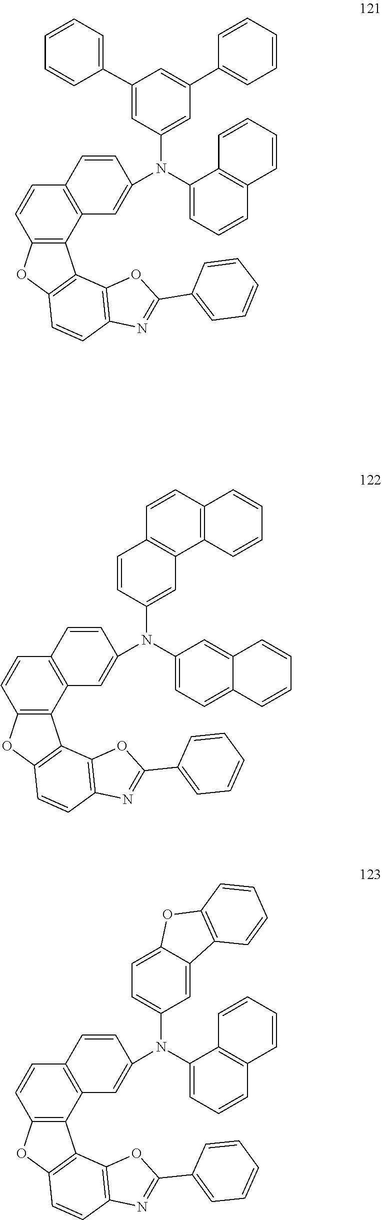 [SMILES: c1ccc(-c2cc(-c3ccccc3)cc(N(c3ccc4ccc5oc6ccc7nc(-c8ccccc8)oc7c6c5c4c3)c3cccc4ccccc34)c2)cc1 ; c1ccc(-c2nc3ccc4oc5ccc6ccc(N(c7ccc8ccccc8c7)c7ccc8ccc9ccccc9c8c7)cc6c5c4c3o2)cc1 ; c1ccc(-c2nc3ccc4oc5ccc6ccc(N(c7ccc8oc9ccccc9c8c7)c7cccc8ccccc78)cc6c5c4c3o2)cc1]

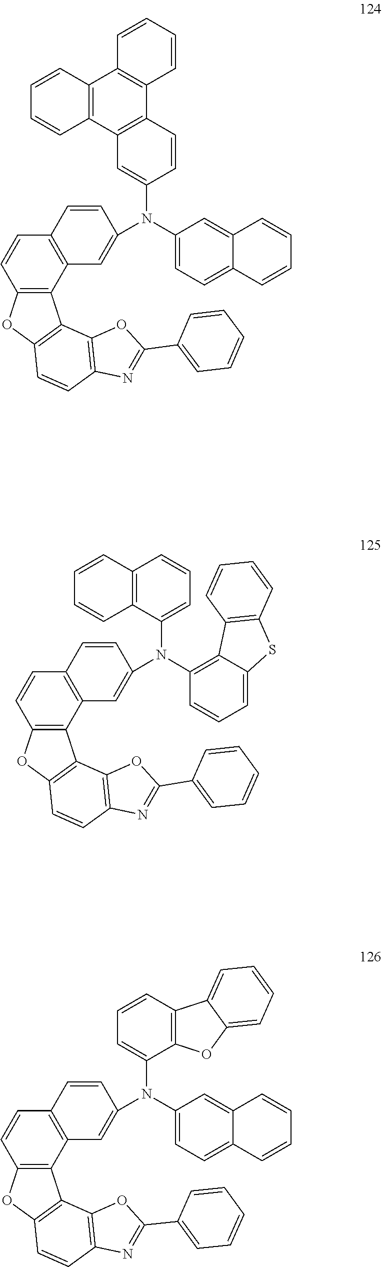 [SMILES: c1ccc(-c2nc3ccc4oc5ccc6ccc(N(c7ccc8ccccc8c7)c7ccc8c9ccccc9c9ccccc9c8c7)cc6c5c4c3o2)cc1 ; c1ccc(-c2nc3ccc4oc5ccc6ccc(N(c7cccc8ccccc78)c7cccc8sc9ccccc9c78)cc6c5c4c3o2)cc1 ; c1ccc(-c2nc3ccc4oc5ccc6ccc(N(c7ccc8ccccc8c7)c7cccc8c7oc7ccccc78)cc6c5c4c3o2)cc1]

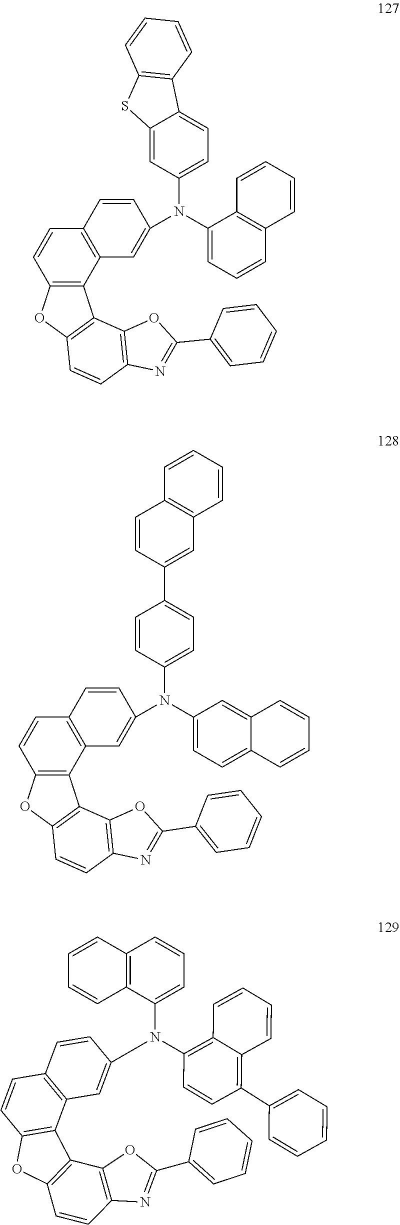 [SMILES: c1ccc(-c2nc3ccc4oc5ccc6ccc(N(c7ccc8c(c7)sc7ccccc78)c7cccc8ccccc78)cc6c5c4c3o2)cc1 ; c1ccc(-c2nc3ccc4oc5ccc6ccc(N(c7ccc(-c8ccc9ccccc9c8)cc7)c7ccc8ccccc8c7)cc6c5c4c3o2)cc1 ; c1ccc(-c2nc3ccc4oc5ccc6ccc(N(c7cccc8ccccc78)c7ccc(-c8ccccc8)c8ccccc78)cc6c5c4c3o2)cc1]

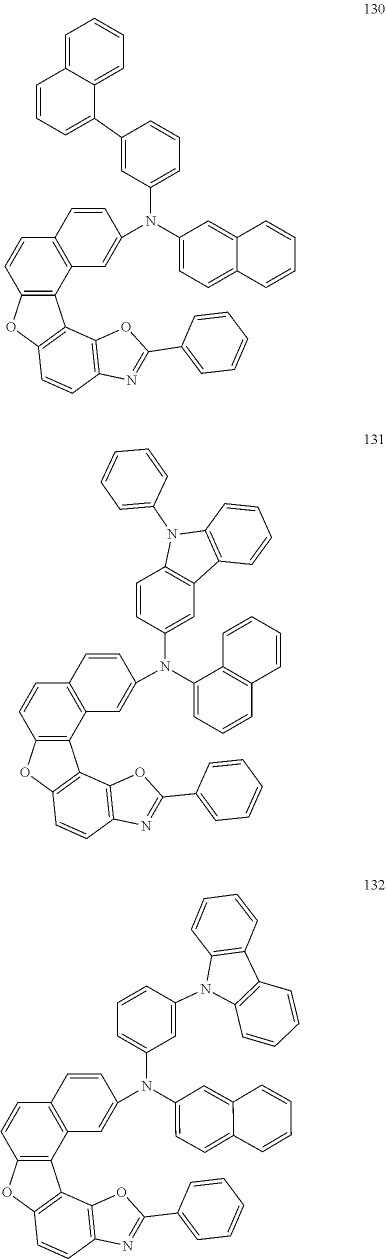 [SMILES: c1ccc(-c2nc3ccc4oc5ccc6ccc(N(c7cccc(-c8cccc9ccccc89)c7)c7ccc8ccccc8c7)cc6c5c4c3o2)cc1 ; c1ccc(-c2nc3ccc4oc5ccc6ccc(N(c7ccc8c(c7)c7ccccc7n8-c7ccccc7)c7cccc8ccccc78)cc6c5c4c3o2)cc1 ; c1ccc(-c2nc3ccc4oc5ccc6ccc(N(c7cccc(-n8c9ccccc9c9ccccc98)c7)c7ccc8ccccc8c7)cc6c5c4c3o2)cc1]

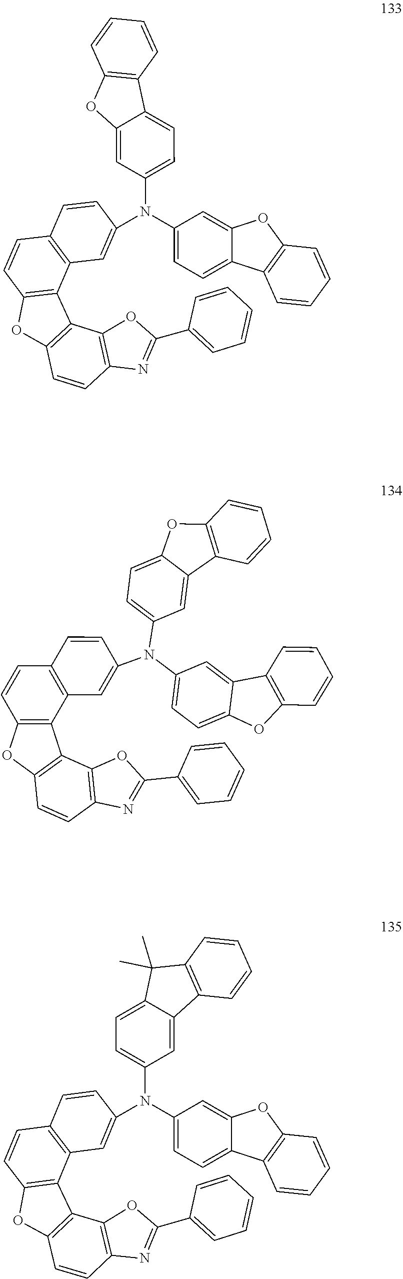 [SMILES: c1ccc(-c2nc3ccc4oc5ccc6ccc(N(c7ccc8c(c7)oc7ccccc78)c7ccc8c(c7)oc7ccccc78)cc6c5c4c3o2)cc1 ; c1ccc(-c2nc3ccc4oc5ccc6ccc(N(c7ccc8oc9ccccc9c8c7)c7ccc8oc9ccccc9c8c7)cc6c5c4c3o2)cc1 ; CC1(C)c2ccccc2-c2cc(N(c3ccc4c(c3)oc3ccccc34)c3ccc4ccc5oc6ccc7nc(-c8ccccc8)oc7c6c5c4c3)ccc21]

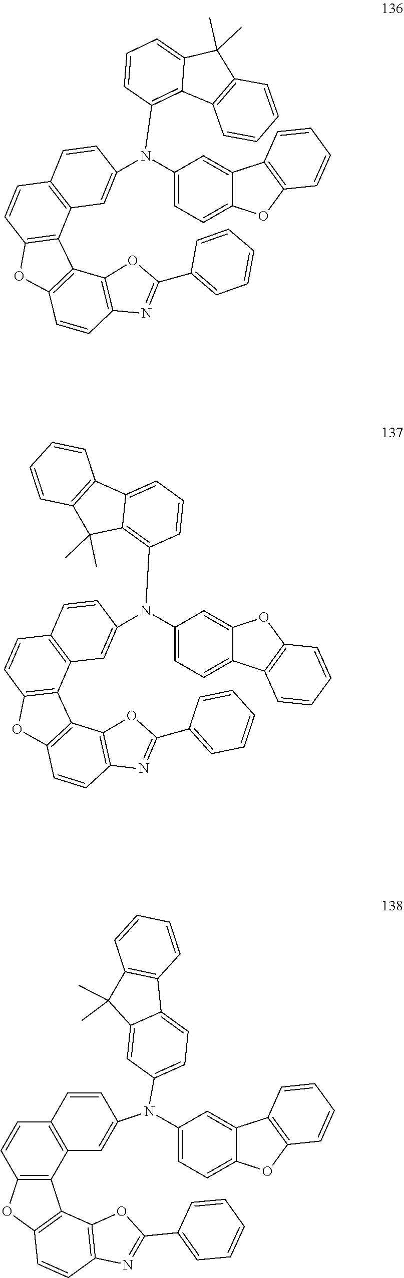 [SMILES: CC1(C)c2ccccc2-c2c(N(c3ccc4oc5ccccc5c4c3)c3ccc4ccc5oc6ccc7nc(-c8ccccc8)oc7c6c5c4c3)cccc21 ; CC1(C)c2ccccc2-c2cccc(N(c3ccc4c(c3)oc3ccccc34)c3ccc4ccc5oc6ccc7nc(-c8ccccc8)oc7c6c5c4c3)c21 ; CC1(C)c2ccccc2-c2ccc(N(c3ccc4oc5ccccc5c4c3)c3ccc4ccc5oc6ccc7nc(-c8ccccc8)oc7c6c5c4c3)cc21]

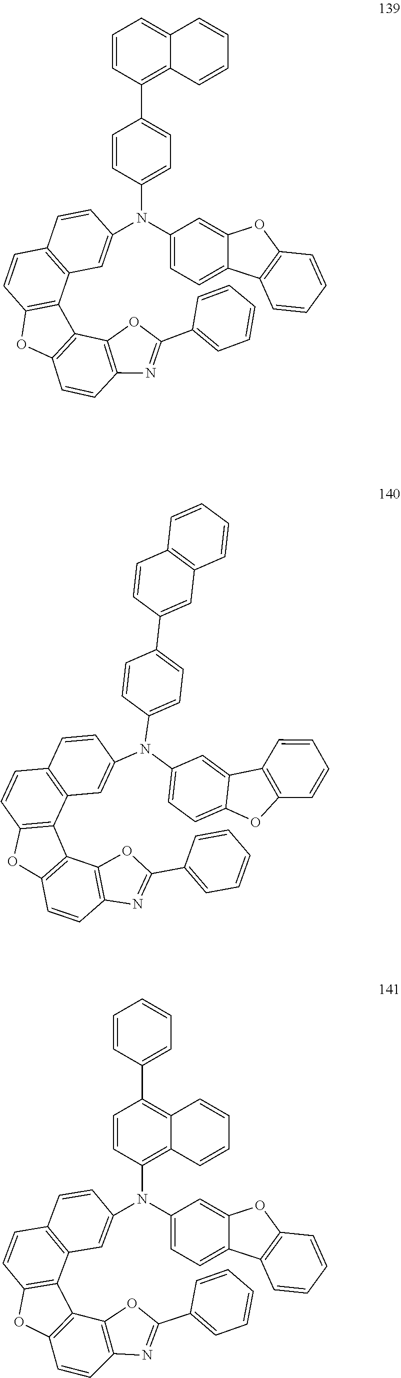 [SMILES: c1ccc(-c2nc3ccc4oc5ccc6ccc(N(c7ccc(-c8cccc9ccccc89)cc7)c7ccc8c(c7)oc7ccccc78)cc6c5c4c3o2)cc1 ; c1ccc(-c2nc3ccc4oc5ccc6ccc(N(c7ccc(-c8ccc9ccccc9c8)cc7)c7ccc8oc9ccccc9c8c7)cc6c5c4c3o2)cc1 ; c1ccc(-c2nc3ccc4oc5ccc6ccc(N(c7ccc8c(c7)oc7ccccc78)c7ccc(-c8ccccc8)c8ccccc78)cc6c5c4c3o2)cc1]

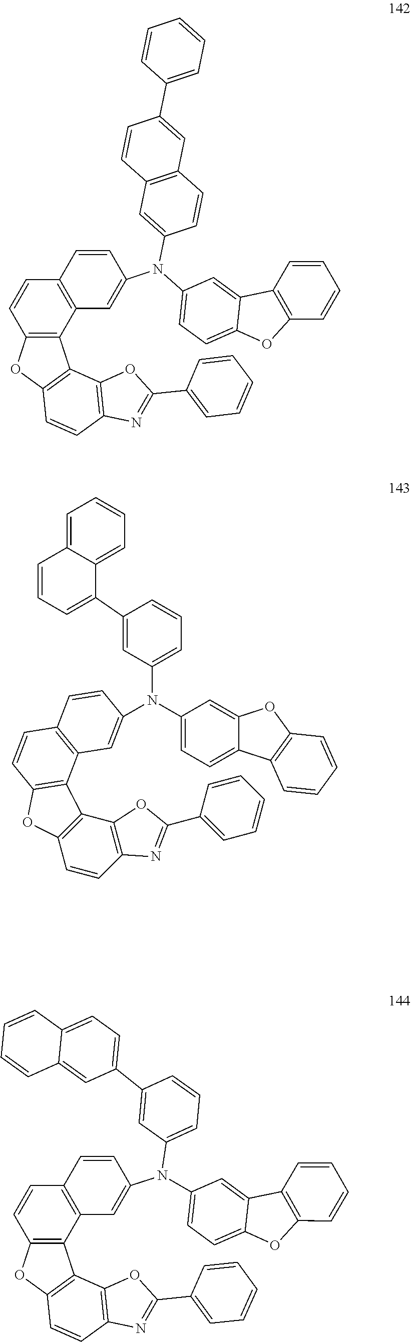 [SMILES: c1ccc(-c2ccc3cc(N(c4ccc5oc6ccccc6c5c4)c4ccc5ccc6oc7ccc8nc(-c9ccccc9)oc8c7c6c5c4)ccc3c2)cc1 ; c1ccc(-c2nc3ccc4oc5ccc6ccc(N(c7cccc(-c8cccc9ccccc89)c7)c7ccc8c(c7)oc7ccccc78)cc6c5c4c3o2)cc1 ; c1ccc(-c2nc3ccc4oc5ccc6ccc(N(c7cccc(-c8ccc9ccccc9c8)c7)c7ccc8oc9ccccc9c8c7)cc6c5c4c3o2)cc1]

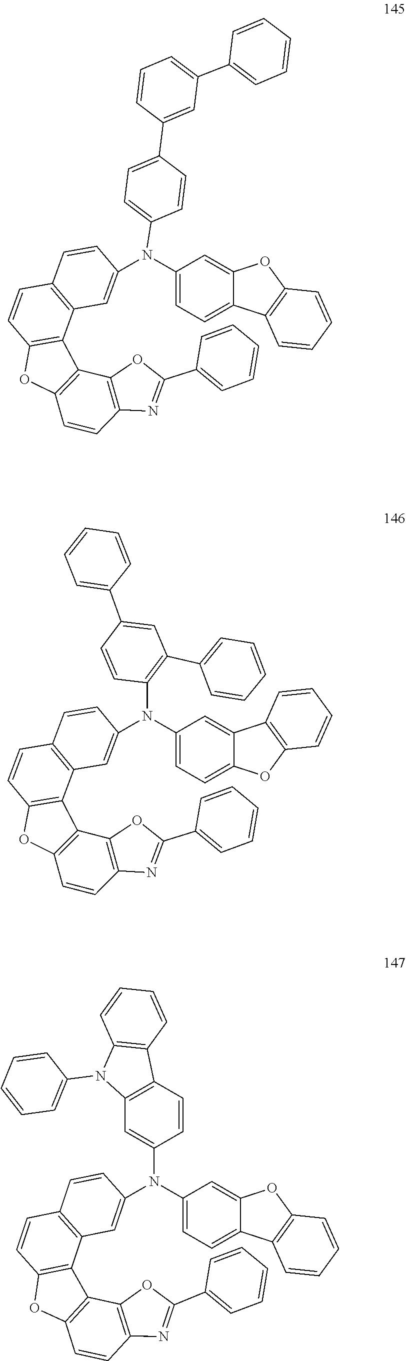 [SMILES: c1ccc(-c2cccc(-c3ccc(N(c4ccc5c(c4)oc4ccccc45)c4ccc5ccc6oc7ccc8nc(-c9ccccc9)oc8c7c6c5c4)cc3)c2)cc1 ; c1ccc(-c2ccc(N(c3ccc4oc5ccccc5c4c3)c3ccc4ccc5oc6ccc7nc(-c8ccccc8)oc7c6c5c4c3)c(-c3ccccc3)c2)cc1 ; c1ccc(-c2nc3ccc4oc5ccc6ccc(N(c7ccc8c(c7)oc7ccccc78)c7ccc8c9ccccc9n(-c9ccccc9)c8c7)cc6c5c4c3o2)cc1]

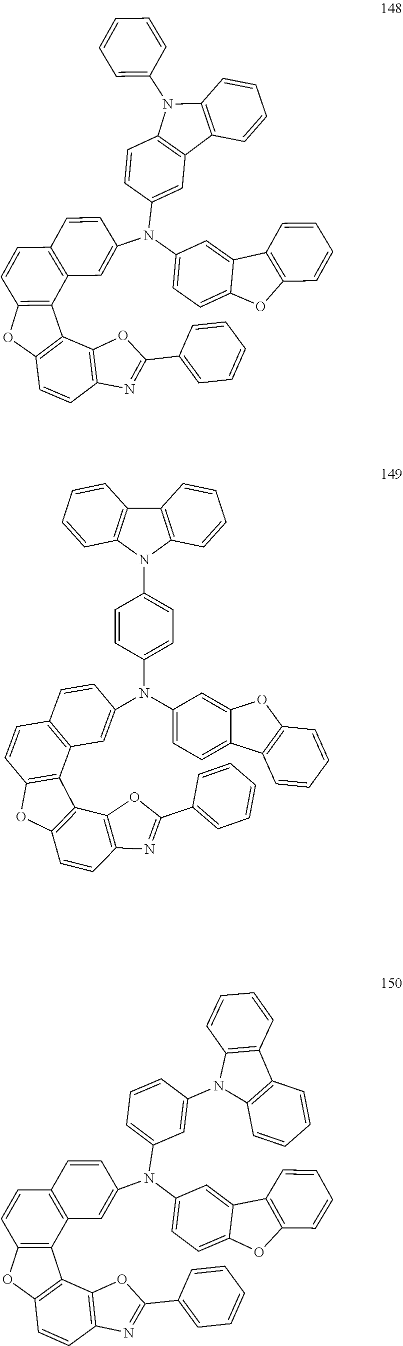 [SMILES: c1ccc(-c2nc3ccc4oc5ccc6ccc(N(c7ccc8oc9ccccc9c8c7)c7ccc8c(c7)c7ccccc7n8-c7ccccc7)cc6c5c4c3o2)cc1 ; c1ccc(-c2nc3ccc4oc5ccc6ccc(N(c7ccc(-n8c9ccccc9c9ccccc98)cc7)c7ccc8c(c7)oc7ccccc78)cc6c5c4c3o2)cc1 ; c1ccc(-c2nc3ccc4oc5ccc6ccc(N(c7cccc(-n8c9ccccc9c9ccccc98)c7)c7ccc8oc9ccccc9c8c7)cc6c5c4c3o2)cc1]

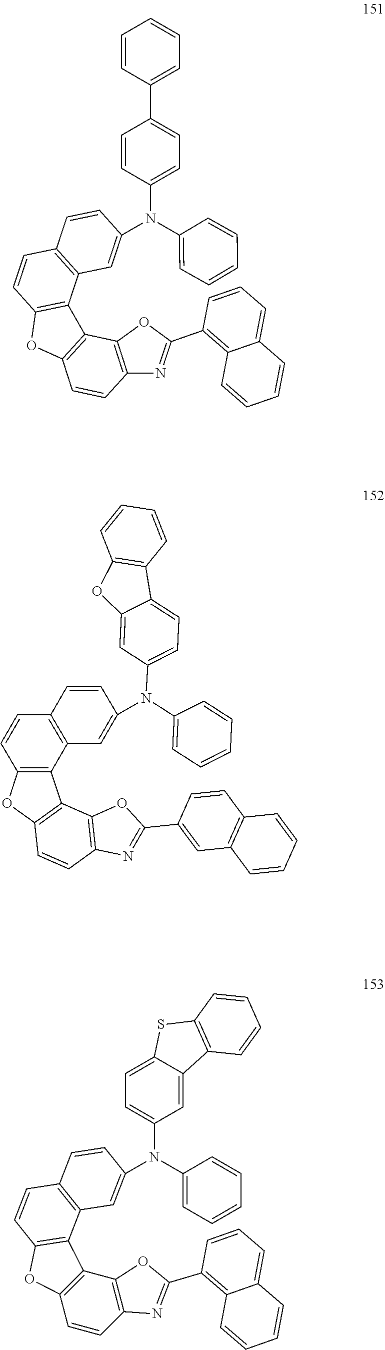 [SMILES: c1ccc(-c2ccc(N(c3ccccc3)c3ccc4ccc5oc6ccc7nc(-c8cccc9ccccc89)oc7c6c5c4c3)cc2)cc1 ; c1ccc(N(c2ccc3c(c2)oc2ccccc23)c2ccc3ccc4oc5ccc6nc(-c7ccc8ccccc8c7)oc6c5c4c3c2)cc1 ; c1ccc(N(c2ccc3sc4ccccc4c3c2)c2ccc3ccc4oc5ccc6nc(-c7cccc8ccccc78)oc6c5c4c3c2)cc1]

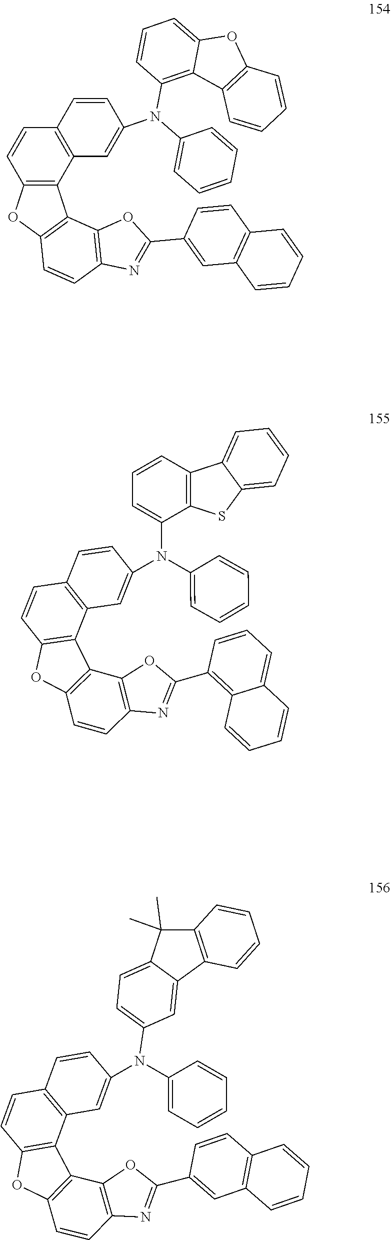 [SMILES: c1ccc(N(c2ccc3ccc4oc5ccc6nc(-c7ccc8ccccc8c7)oc6c5c4c3c2)c2cccc3oc4ccccc4c23)cc1 ; c1ccc(N(c2ccc3ccc4oc5ccc6nc(-c7cccc8ccccc78)oc6c5c4c3c2)c2cccc3c2sc2ccccc23)cc1 ; CC1(C)c2ccccc2-c2cc(N(c3ccccc3)c3ccc4ccc5oc6ccc7nc(-c8ccc9ccccc9c8)oc7c6c5c4c3)ccc21]

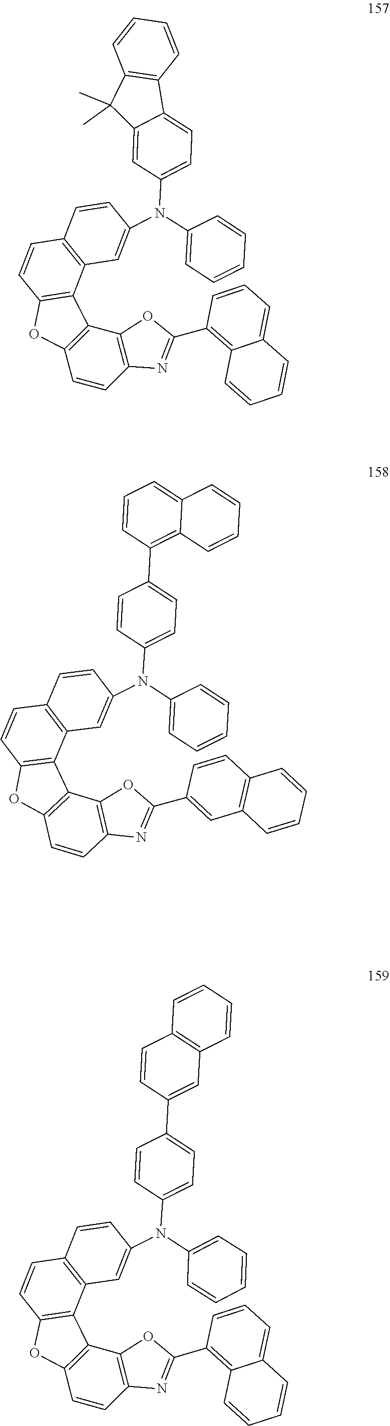 [SMILES: CC1(C)c2ccccc2-c2ccc(N(c3ccccc3)c3ccc4ccc5oc6ccc7nc(-c8cccc9ccccc89)oc7c6c5c4c3)cc21 ; c1ccc(N(c2ccc(-c3cccc4ccccc34)cc2)c2ccc3ccc4oc5ccc6nc(-c7ccc8ccccc8c7)oc6c5c4c3c2)cc1 ; c1ccc(N(c2ccc(-c3ccc4ccccc4c3)cc2)c2ccc3ccc4oc5ccc6nc(-c7cccc8ccccc78)oc6c5c4c3c2)cc1]

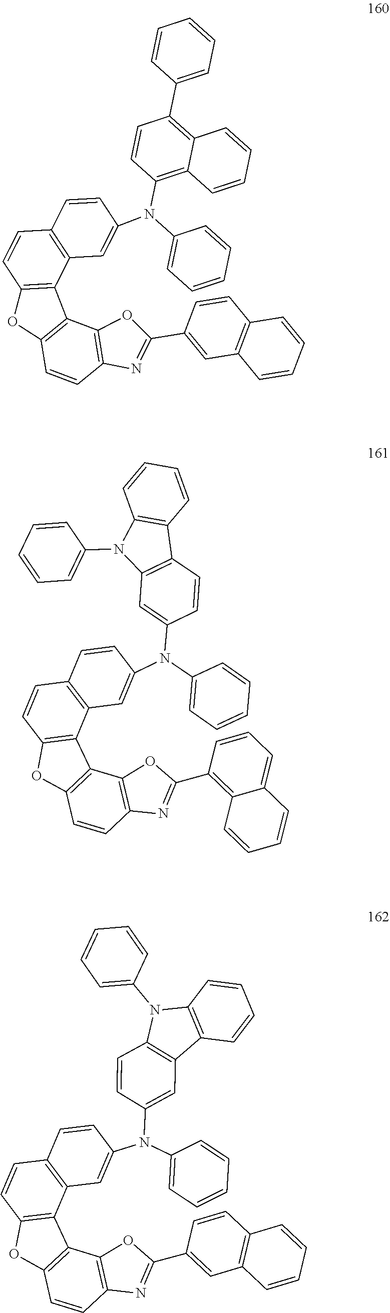 [SMILES: c1ccc(-c2ccc(N(c3ccccc3)c3ccc4ccc5oc6ccc7nc(-c8ccc9ccccc9c8)oc7c6c5c4c3)c3ccccc23)cc1 ; c1ccc(N(c2ccc3ccc4oc5ccc6nc(-c7cccc8ccccc78)oc6c5c4c3c2)c2ccc3c4ccccc4n(-c4ccccc4)c3c2)cc1 ; c1ccc(N(c2ccc3ccc4oc5ccc6nc(-c7ccc8ccccc8c7)oc6c5c4c3c2)c2ccc3c(c2)c2ccccc2n3-c2ccccc2)cc1]

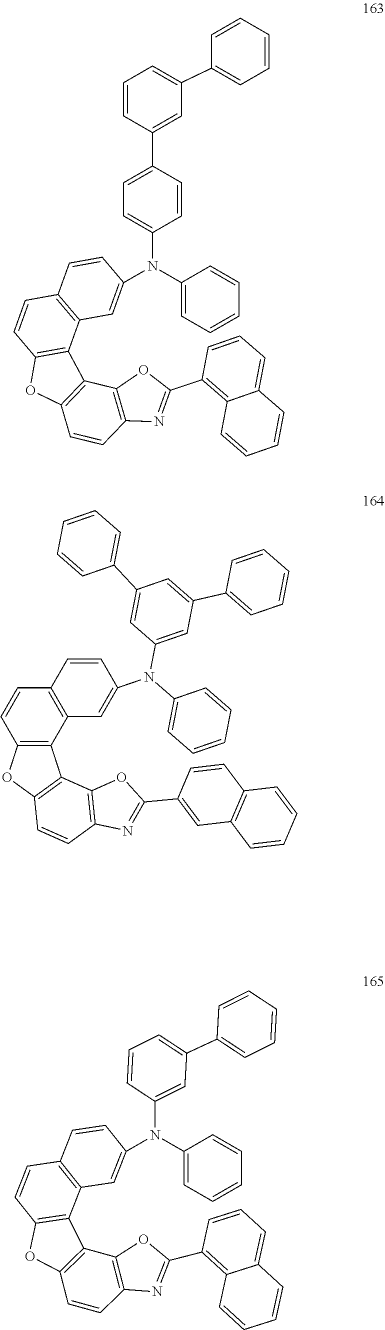 [SMILES: c1ccc(-c2cccc(-c3ccc(N(c4ccccc4)c4ccc5ccc6oc7ccc8nc(-c9cccc%10ccccc9%10)oc8c7c6c5c4)cc3)c2)cc1 ; c1ccc(-c2cc(-c3ccccc3)cc(N(c3ccccc3)c3ccc4ccc5oc6ccc7nc(-c8ccc9ccccc9c8)oc7c6c5c4c3)c2)cc1 ; c1ccc(-c2cccc(N(c3ccccc3)c3ccc4ccc5oc6ccc7nc(-c8cccc9ccccc89)oc7c6c5c4c3)c2)cc1]

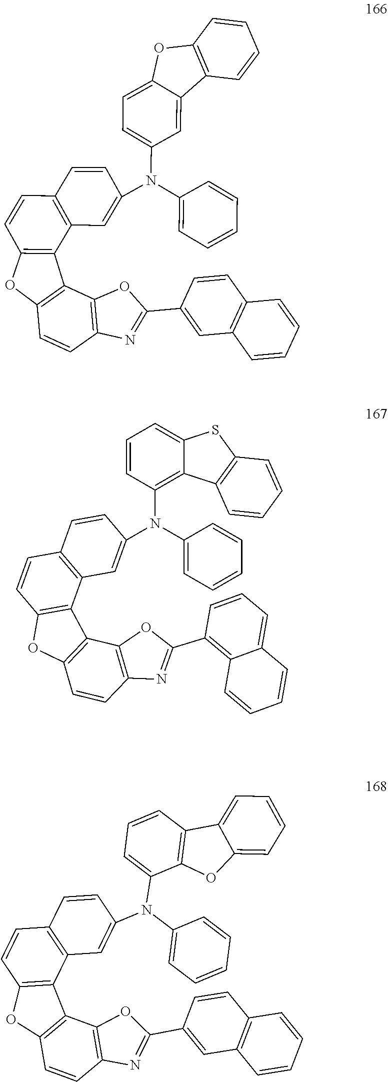 [SMILES: c1ccc(N(c2ccc3oc4ccccc4c3c2)c2ccc3ccc4oc5ccc6nc(-c7ccc8ccccc8c7)oc6c5c4c3c2)cc1 ; c1ccc(N(c2ccc3ccc4oc5ccc6nc(-c7cccc8ccccc78)oc6c5c4c3c2)c2cccc3sc4ccccc4c23)cc1 ; c1ccc(N(c2ccc3ccc4oc5ccc6nc(-c7ccc8ccccc8c7)oc6c5c4c3c2)c2cccc3c2oc2ccccc23)cc1]

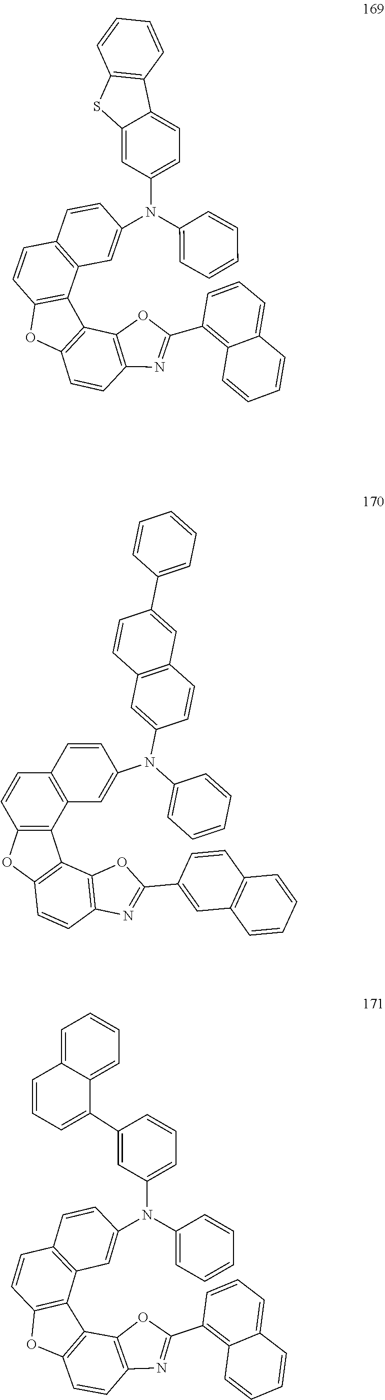 [SMILES: c1ccc(N(c2ccc3c(c2)sc2ccccc23)c2ccc3ccc4oc5ccc6nc(-c7cccc8ccccc78)oc6c5c4c3c2)cc1 ; c1ccc(-c2ccc3cc(N(c4ccccc4)c4ccc5ccc6oc7ccc8nc(-c9ccc%10ccccc%10c9)oc8c7c6c5c4)ccc3c2)cc1 ; c1ccc(N(c2cccc(-c3cccc4ccccc34)c2)c2ccc3ccc4oc5ccc6nc(-c7cccc8ccccc78)oc6c5c4c3c2)cc1]

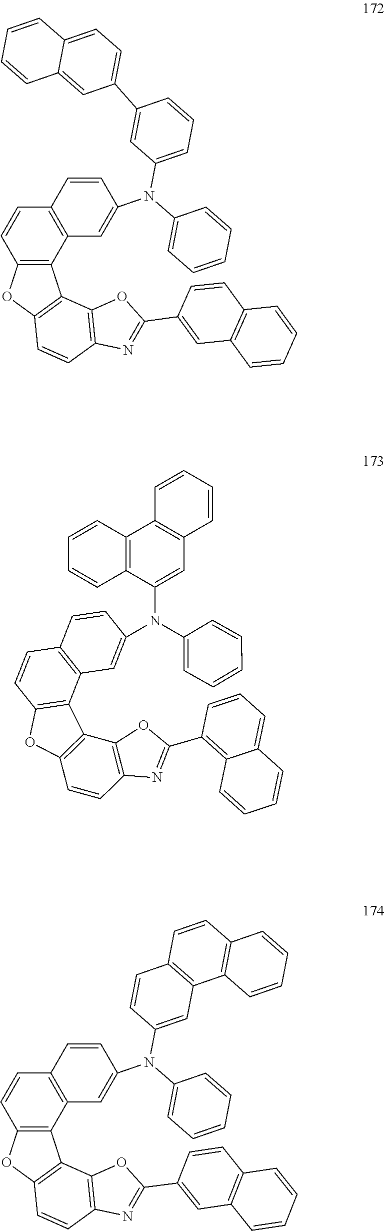 [SMILES: c1ccc(N(c2cccc(-c3ccc4ccccc4c3)c2)c2ccc3ccc4oc5ccc6nc(-c7ccc8ccccc8c7)oc6c5c4c3c2)cc1 ; c1ccc(N(c2ccc3ccc4oc5ccc6nc(-c7cccc8ccccc78)oc6c5c4c3c2)c2cc3ccccc3c3ccccc23)cc1 ; c1ccc(N(c2ccc3ccc4ccccc4c3c2)c2ccc3ccc4oc5ccc6nc(-c7ccc8ccccc8c7)oc6c5c4c3c2)cc1]

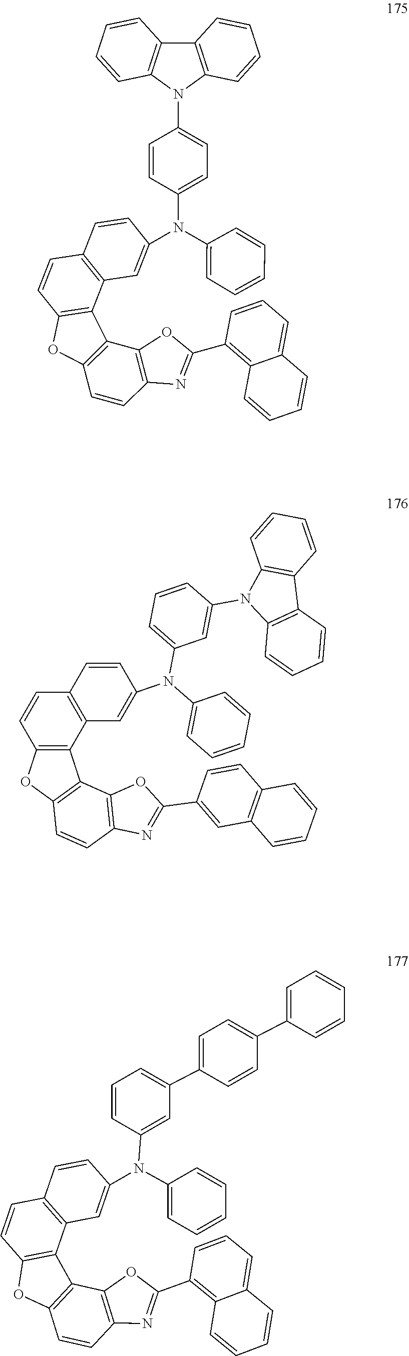 [SMILES: c1ccc(N(c2ccc(-n3c4ccccc4c4ccccc43)cc2)c2ccc3ccc4oc5ccc6nc(-c7cccc8ccccc78)oc6c5c4c3c2)cc1 ; c1ccc(N(c2cccc(-n3c4ccccc4c4ccccc43)c2)c2ccc3ccc4oc5ccc6nc(-c7ccc8ccccc8c7)oc6c5c4c3c2)cc1 ; c1ccc(-c2ccc(-c3cccc(N(c4ccccc4)c4ccc5ccc6oc7ccc8nc(-c9cccc%10ccccc9%10)oc8c7c6c5c4)c3)cc2)cc1]

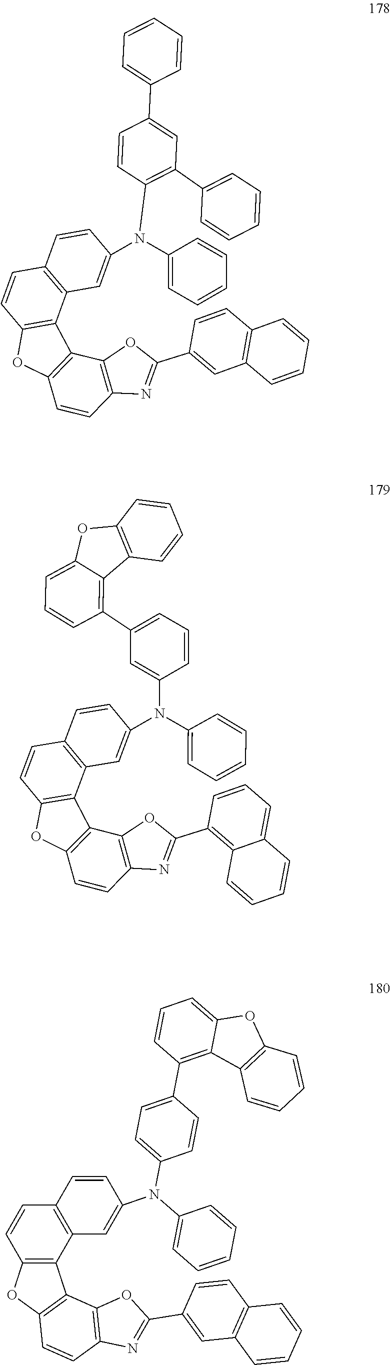 [SMILES: c1ccc(-c2ccc(N(c3ccccc3)c3ccc4ccc5oc6ccc7nc(-c8ccc9ccccc9c8)oc7c6c5c4c3)c(-c3ccccc3)c2)cc1 ; c1ccc(N(c2cccc(-c3cccc4oc5ccccc5c34)c2)c2ccc3ccc4oc5ccc6nc(-c7cccc8ccccc78)oc6c5c4c3c2)cc1 ; c1ccc(N(c2ccc(-c3cccc4oc5ccccc5c34)cc2)c2ccc3ccc4oc5ccc6nc(-c7ccc8ccccc8c7)oc6c5c4c3c2)cc1]

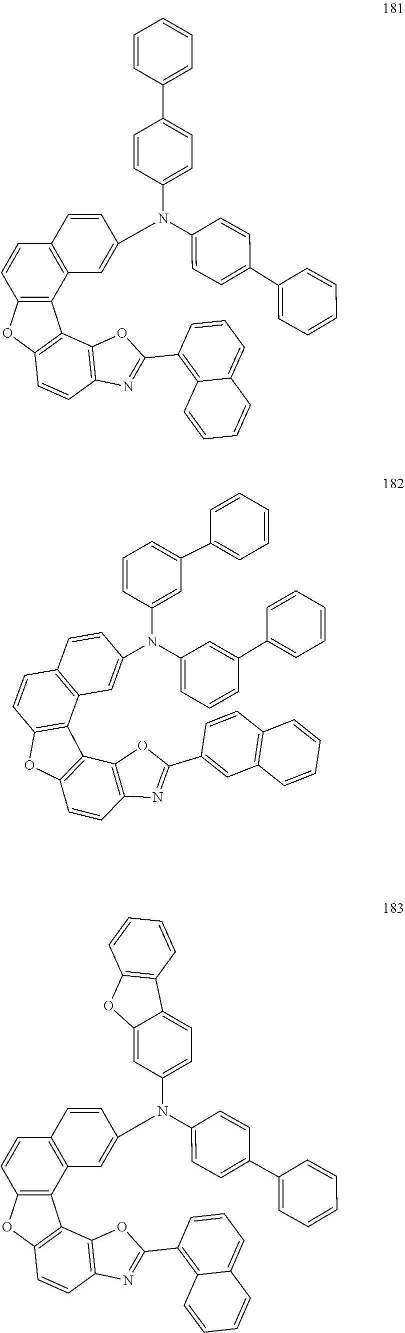 [SMILES: c1ccc(-c2ccc(N(c3ccc(-c4ccccc4)cc3)c3ccc4ccc5oc6ccc7nc(-c8cccc9ccccc89)oc7c6c5c4c3)cc2)cc1 ; c1ccc(-c2cccc(N(c3cccc(-c4ccccc4)c3)c3ccc4ccc5oc6ccc7nc(-c8ccc9ccccc9c8)oc7c6c5c4c3)c2)cc1 ; c1ccc(-c2ccc(N(c3ccc4c(c3)oc3ccccc34)c3ccc4ccc5oc6ccc7nc(-c8cccc9ccccc89)oc7c6c5c4c3)cc2)cc1]

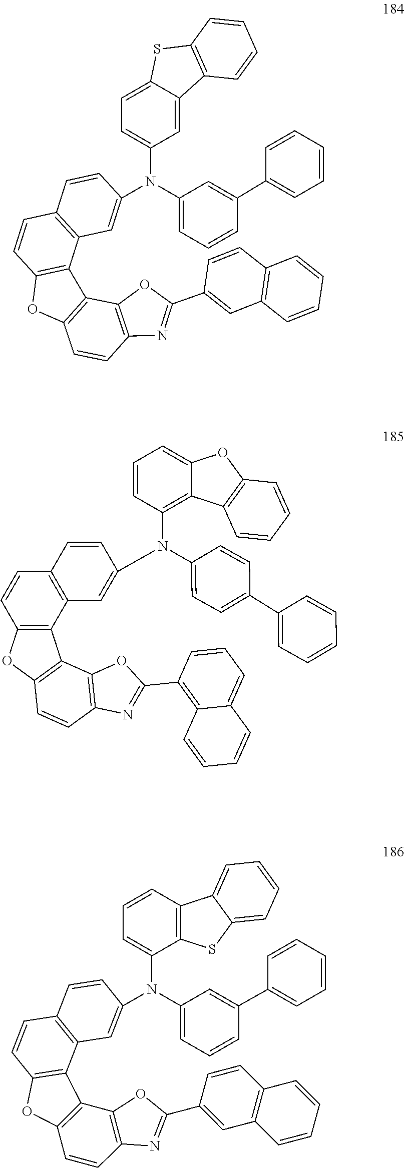 [SMILES: c1ccc(-c2cccc(N(c3ccc4sc5ccccc5c4c3)c3ccc4ccc5oc6ccc7nc(-c8ccc9ccccc9c8)oc7c6c5c4c3)c2)cc1 ; c1ccc(-c2ccc(N(c3ccc4ccc5oc6ccc7nc(-c8cccc9ccccc89)oc7c6c5c4c3)c3cccc4oc5ccccc5c34)cc2)cc1 ; c1ccc(-c2cccc(N(c3ccc4ccc5oc6ccc7nc(-c8ccc9ccccc9c8)oc7c6c5c4c3)c3cccc4c3sc3ccccc34)c2)cc1]

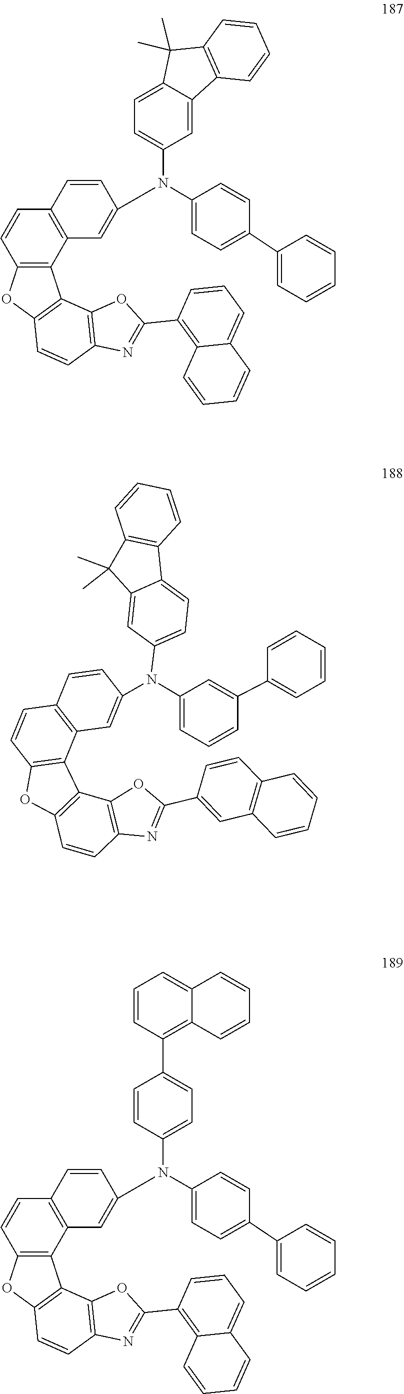 [SMILES: CC1(C)c2ccccc2-c2cc(N(c3ccc(-c4ccccc4)cc3)c3ccc4ccc5oc6ccc7nc(-c8cccc9ccccc89)oc7c6c5c4c3)ccc21 ; CC1(C)c2ccccc2-c2ccc(N(c3cccc(-c4ccccc4)c3)c3ccc4ccc5oc6ccc7nc(-c8ccc9ccccc9c8)oc7c6c5c4c3)cc21 ; c1ccc(-c2ccc(N(c3ccc(-c4cccc5ccccc45)cc3)c3ccc4ccc5oc6ccc7nc(-c8cccc9ccccc89)oc7c6c5c4c3)cc2)cc1]

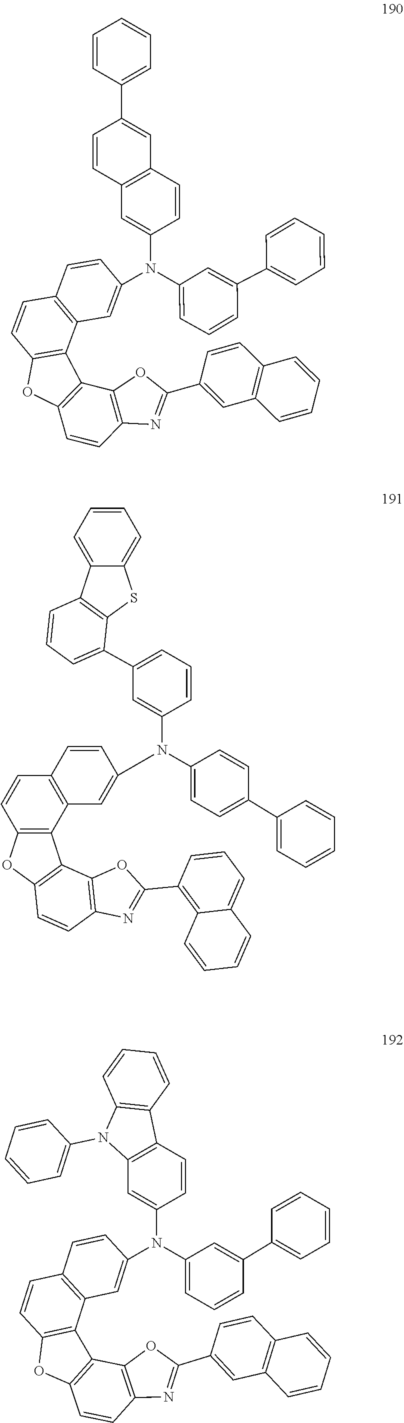 [SMILES: c1ccc(-c2cccc(N(c3ccc4cc(-c5ccccc5)ccc4c3)c3ccc4ccc5oc6ccc7nc(-c8ccc9ccccc9c8)oc7c6c5c4c3)c2)cc1 ; c1ccc(-c2ccc(N(c3cccc(-c4cccc5c4sc4ccccc45)c3)c3ccc4ccc5oc6ccc7nc(-c8cccc9ccccc89)oc7c6c5c4c3)cc2)cc1 ; c1ccc(-c2cccc(N(c3ccc4ccc5oc6ccc7nc(-c8ccc9ccccc9c8)oc7c6c5c4c3)c3ccc4c5ccccc5n(-c5ccccc5)c4c3)c2)cc1]

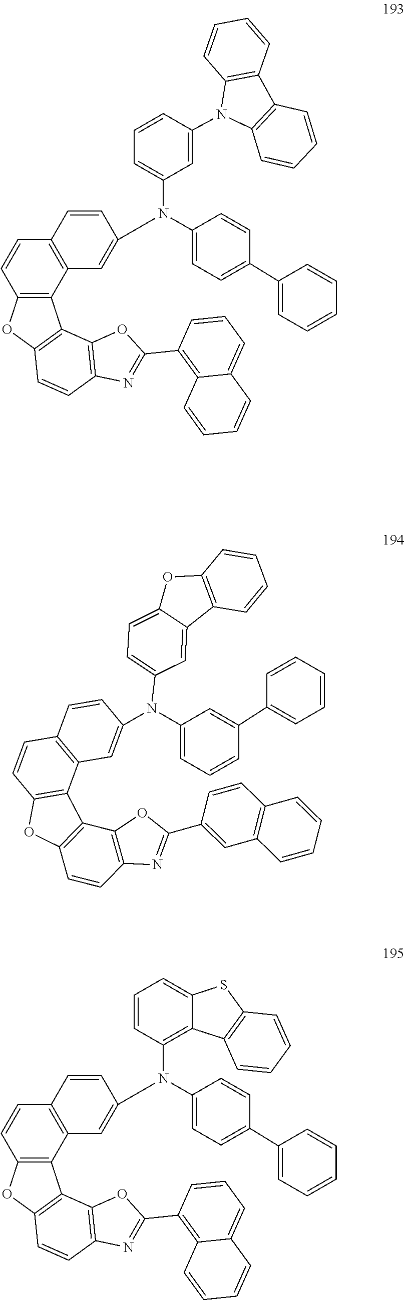 [SMILES: c1ccc(-c2ccc(N(c3cccc(-n4c5ccccc5c5ccccc54)c3)c3ccc4ccc5oc6ccc7nc(-c8cccc9ccccc89)oc7c6c5c4c3)cc2)cc1 ; c1ccc(-c2cccc(N(c3ccc4oc5ccccc5c4c3)c3ccc4ccc5oc6ccc7nc(-c8ccc9ccccc9c8)oc7c6c5c4c3)c2)cc1 ; c1ccc(-c2ccc(N(c3ccc4ccc5oc6ccc7nc(-c8cccc9ccccc89)oc7c6c5c4c3)c3cccc4sc5ccccc5c34)cc2)cc1]

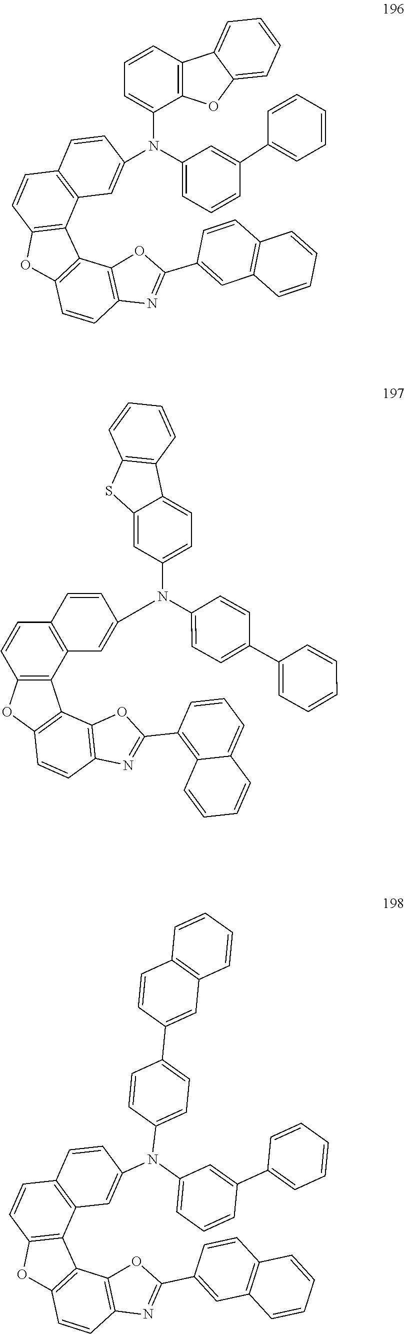 [SMILES: c1ccc(-c2cccc(N(c3ccc4ccc5oc6ccc7nc(-c8ccc9ccccc9c8)oc7c6c5c4c3)c3cccc4c3oc3ccccc34)c2)cc1 ; c1ccc(-c2ccc(N(c3ccc4c(c3)sc3ccccc34)c3ccc4ccc5oc6ccc7nc(-c8cccc9ccccc89)oc7c6c5c4c3)cc2)cc1 ; c1ccc(-c2cccc(N(c3ccc(-c4ccc5ccccc5c4)cc3)c3ccc4ccc5oc6ccc7nc(-c8ccc9ccccc9c8)oc7c6c5c4c3)c2)cc1]

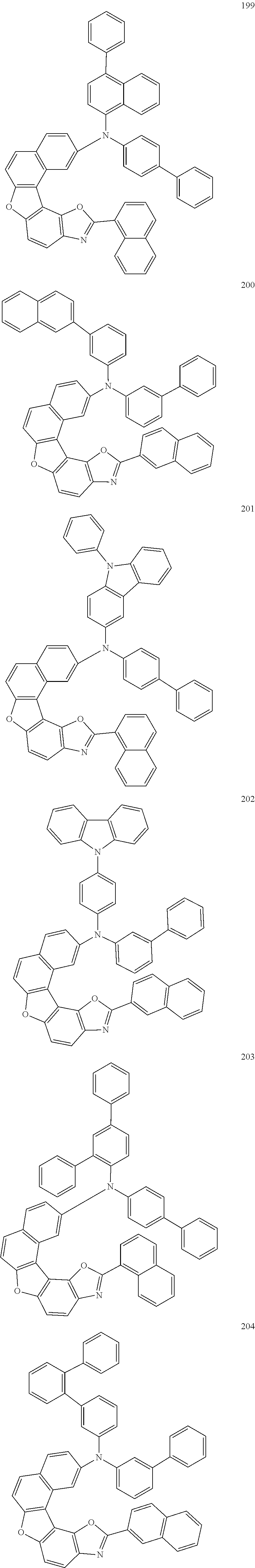

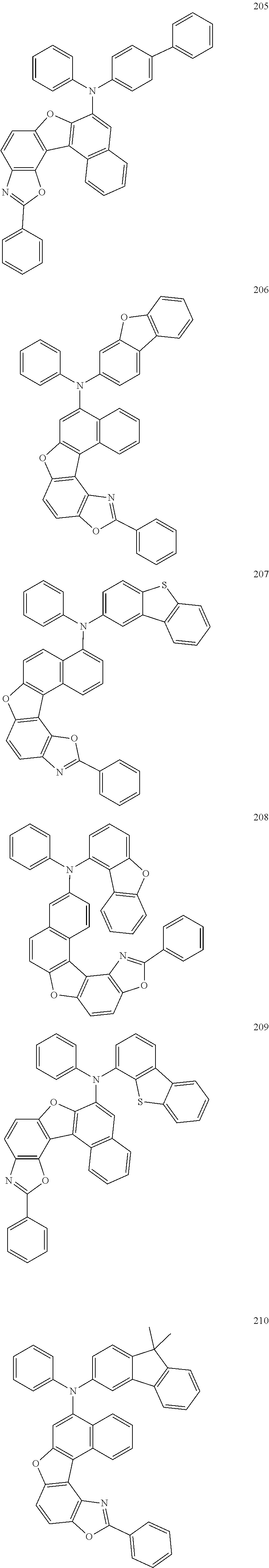

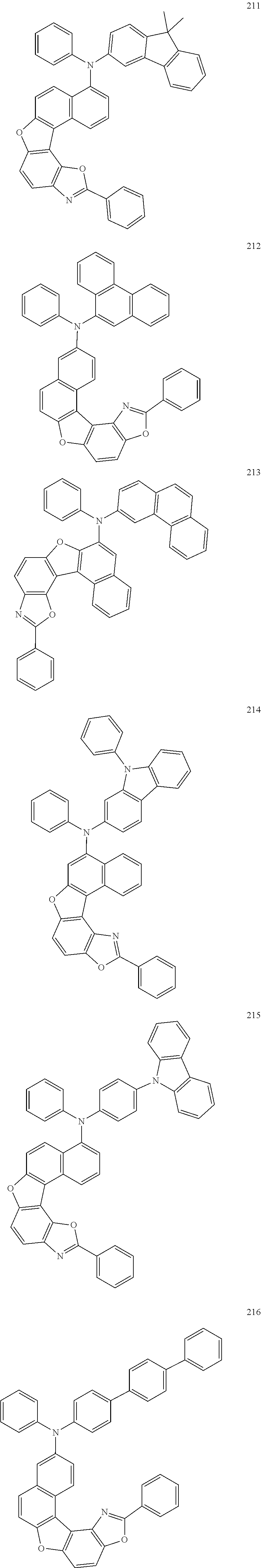

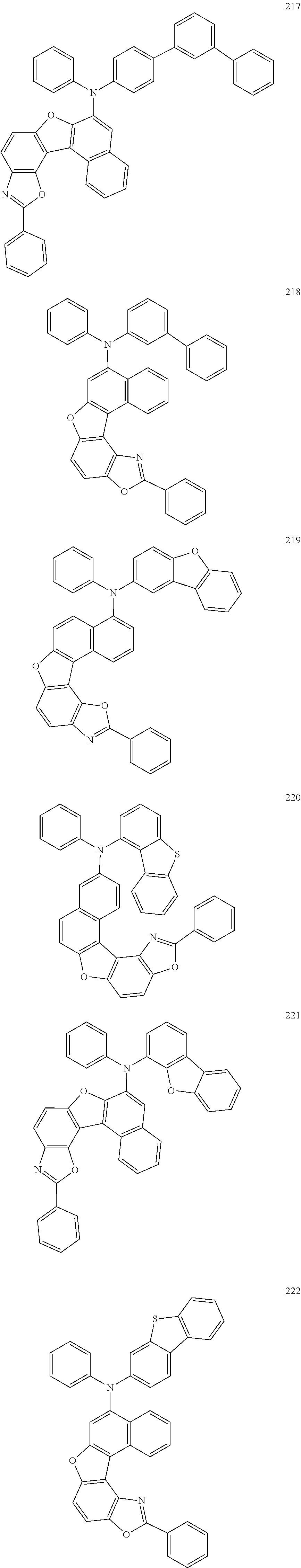

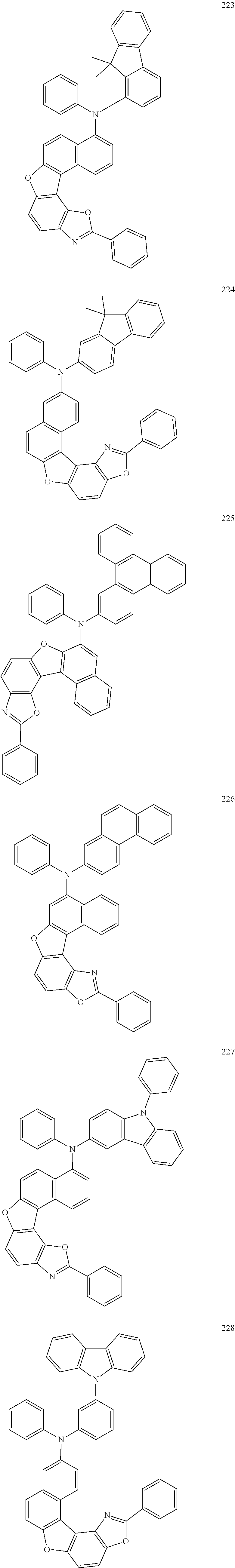

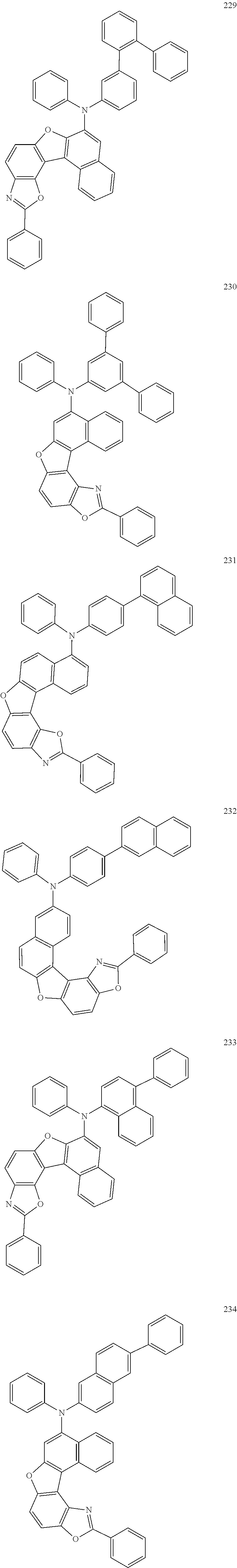 [SMILES: c1ccc(-c2nc3ccc4oc5c(N(c6ccccc6)c6cccc(-c7ccccc7-c7ccccc7)c6)cc6ccccc6c5c4c3o2)cc1 ; c1ccc(-c2cc(-c3ccccc3)cc(N(c3ccccc3)c3cc4oc5ccc6oc(-c7ccccc7)nc6c5c4c4ccccc34)c2)cc1 ; c1ccc(-c2nc3ccc4oc5ccc6c(N(c7ccccc7)c7ccc(-c8cccc9ccccc89)cc7)cccc6c5c4c3o2)cc1 ; c1ccc(-c2nc3c(ccc4oc5ccc6cc(N(c7ccccc7)c7ccc(-c8ccc9ccccc9c8)cc7)ccc6c5c43)o2)cc1 ; c1ccc(-c2nc3ccc4oc5c(N(c6ccccc6)c6ccc(-c7ccccc7)c7ccccc67)cc6ccccc6c5c4c3o2)cc1 ; c1ccc(-c2ccc3cc(N(c4ccccc4)c4cc5oc6ccc7oc(-c8ccccc8)nc7c6c5c5ccccc45)ccc3c2)cc1]

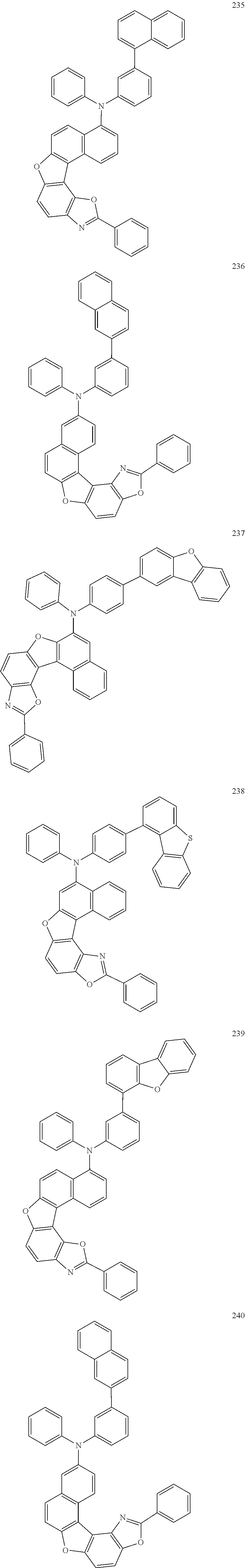

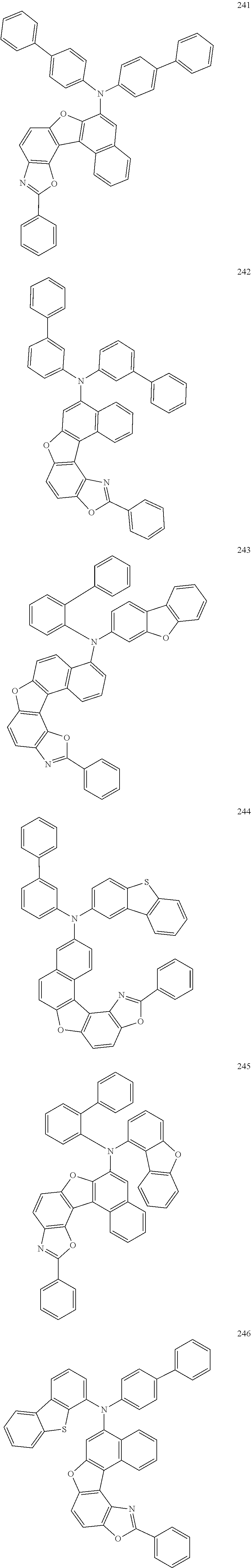

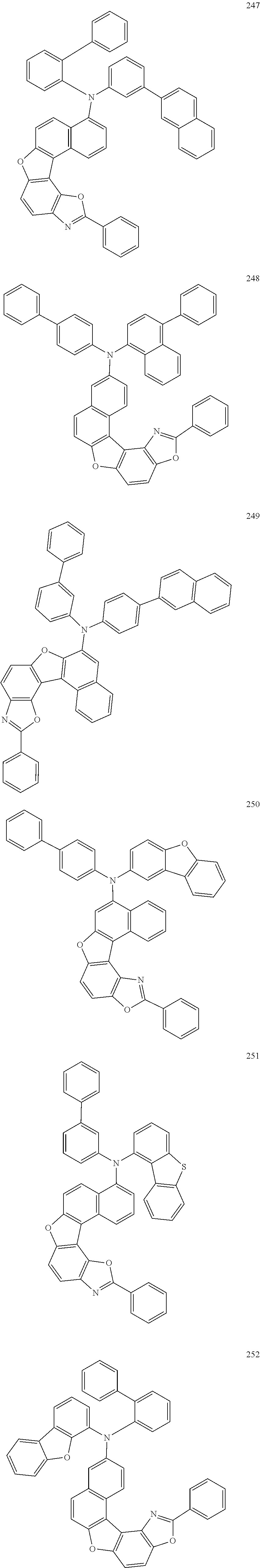

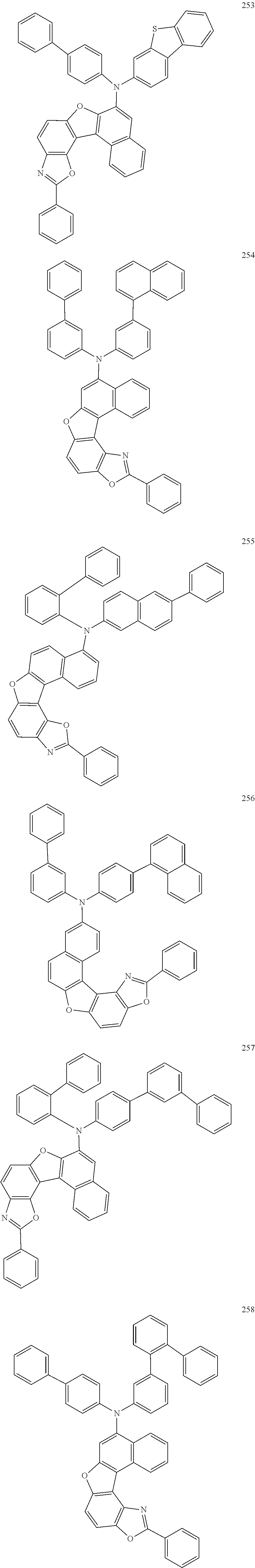

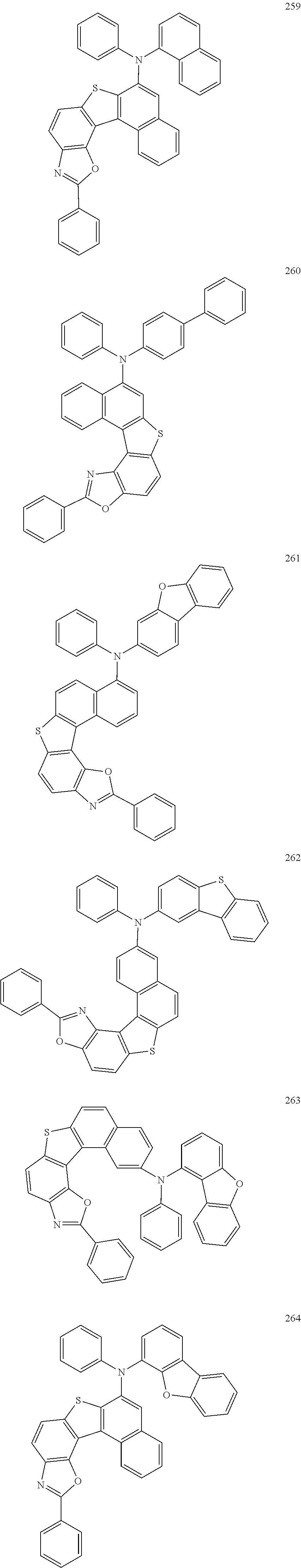 [SMILES: c1ccc(-c2nc3ccc4sc5c(N(c6ccccc6)c6cccc7ccccc67)cc6ccccc6c5c4c3o2)cc1 ; c1ccc(-c2ccc(N(c3ccccc3)c3cc4sc5ccc6oc(-c7ccccc7)nc6c5c4c4ccccc34)cc2)cc1 ; c1ccc(-c2nc3ccc4sc5ccc6c(N(c7ccccc7)c7ccc8c(c7)oc7ccccc78)cccc6c5c4c3o2)cc1 ; c1ccc(-c2nc3c(ccc4sc5ccc6cc(N(c7ccccc7)c7ccc8sc9ccccc9c8c7)ccc6c5c43)o2)cc1 ; c1ccc(-c2nc3ccc4sc5ccc6ccc(N(c7ccccc7)c7cccc8oc9ccccc9c78)cc6c5c4c3o2)cc1 ; c1ccc(-c2nc3ccc4sc5c(N(c6ccccc6)c6cccc7c6oc6ccccc67)cc6ccccc6c5c4c3o2)cc1]

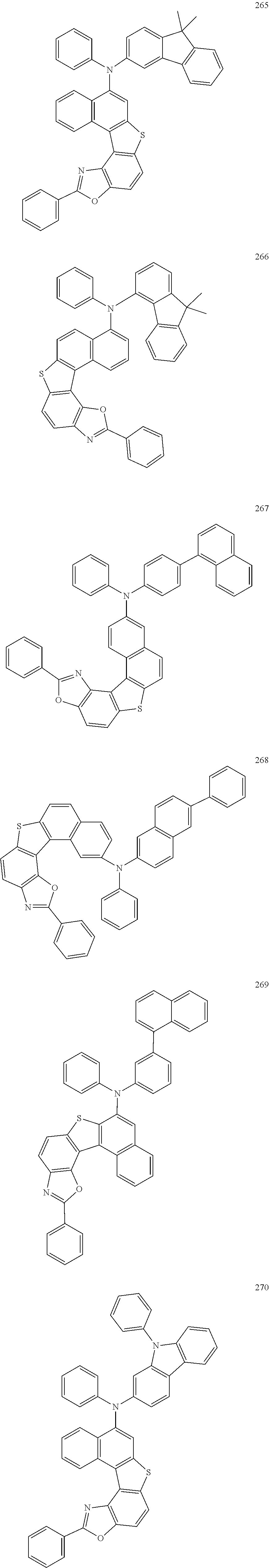 [SMILES: CC1(C)c2ccccc2-c2cc(N(c3ccccc3)c3cc4sc5ccc6oc(-c7ccccc7)nc6c5c4c4ccccc34)ccc21 ; CC1(C)c2ccccc2-c2c(N(c3ccccc3)c3cccc4c3ccc3sc5ccc6nc(-c7ccccc7)oc6c5c34)cccc21 ; c1ccc(-c2nc3c(ccc4sc5ccc6cc(N(c7ccccc7)c7ccc(-c8cccc9ccccc89)cc7)ccc6c5c43)o2)cc1 ; c1ccc(-c2ccc3cc(N(c4ccccc4)c4ccc5ccc6sc7ccc8nc(-c9ccccc9)oc8c7c6c5c4)ccc3c2)cc1 ; c1ccc(-c2nc3ccc4sc5c(N(c6ccccc6)c6cccc(-c7cccc8ccccc78)c6)cc6ccccc6c5c4c3o2)cc1 ; c1ccc(-c2nc3c(ccc4sc5cc(N(c6ccccc6)c6ccc7c8ccccc8n(-c8ccccc8)c7c6)c6ccccc6c5c43)o2)cc1]

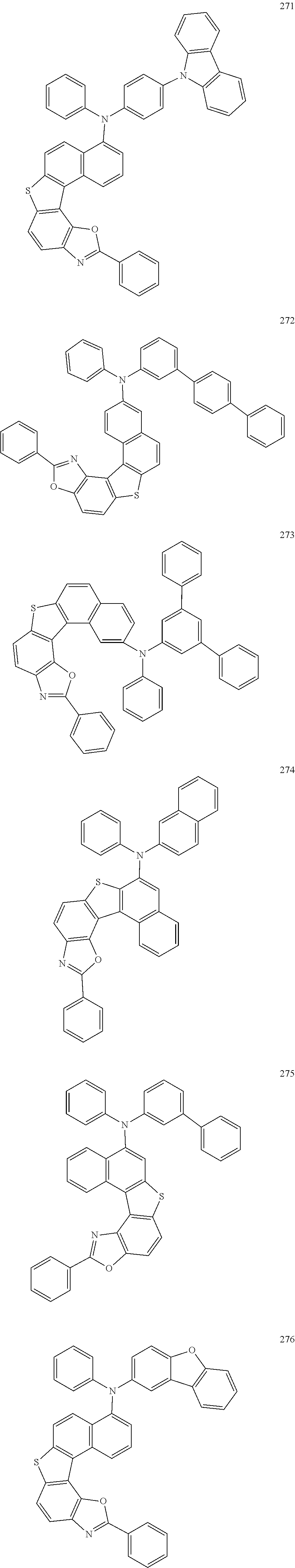 [SMILES: c1ccc(-c2nc3ccc4sc5ccc6c(N(c7ccccc7)c7ccc(-n8c9ccccc9c9ccccc98)cc7)cccc6c5c4c3o2)cc1 ; c1ccc(-c2ccc(-c3cccc(N(c4ccccc4)c4ccc5c(ccc6sc7ccc8oc(-c9ccccc9)nc8c7c65)c4)c3)cc2)cc1 ; c1ccc(-c2cc(-c3ccccc3)cc(N(c3ccccc3)c3ccc4ccc5sc6ccc7nc(-c8ccccc8)oc7c6c5c4c3)c2)cc1 ; c1ccc(-c2nc3ccc4sc5c(N(c6ccccc6)c6ccc7ccccc7c6)cc6ccccc6c5c4c3o2)cc1 ; c1ccc(-c2cccc(N(c3ccccc3)c3cc4sc5ccc6oc(-c7ccccc7)nc6c5c4c4ccccc34)c2)cc1 ; c1ccc(-c2nc3ccc4sc5ccc6c(N(c7ccccc7)c7ccc8oc9ccccc9c8c7)cccc6c5c4c3o2)cc1]

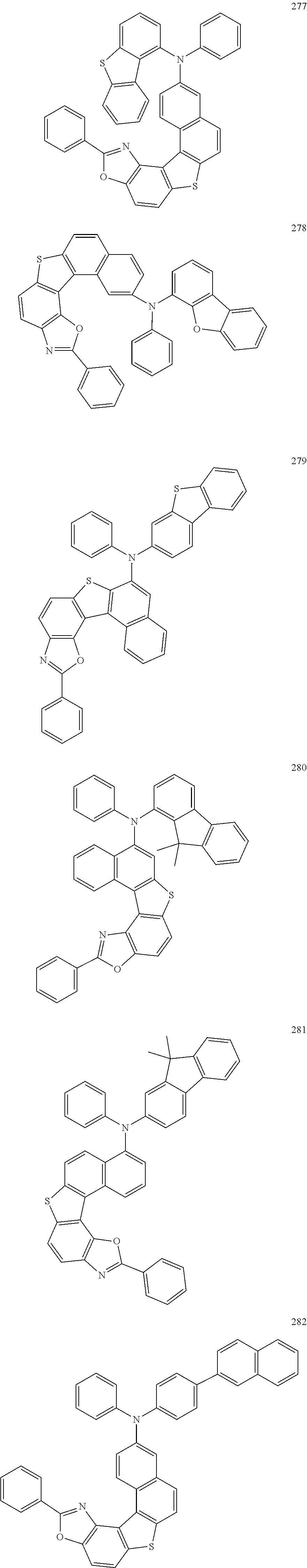 [SMILES: c1ccc(-c2nc3c(ccc4sc5ccc6cc(N(c7ccccc7)c7cccc8sc9ccccc9c78)ccc6c5c43)o2)cc1 ; c1ccc(-c2nc3ccc4sc5ccc6ccc(N(c7ccccc7)c7cccc8c7oc7ccccc78)cc6c5c4c3o2)cc1 ; c1ccc(-c2nc3ccc4sc5c(N(c6ccccc6)c6ccc7c(c6)sc6ccccc67)cc6ccccc6c5c4c3o2)cc1 ; CC1(C)c2ccccc2-c2cccc(N(c3ccccc3)c3cc4sc5ccc6oc(-c7ccccc7)nc6c5c4c4ccccc34)c21 ; CC1(C)c2ccccc2-c2ccc(N(c3ccccc3)c3cccc4c3ccc3sc5ccc6nc(-c7ccccc7)oc6c5c34)cc21 ; c1ccc(-c2nc3c(ccc4sc5ccc6cc(N(c7ccccc7)c7ccc(-c8ccc9ccccc9c8)cc7)ccc6c5c43)o2)cc1]

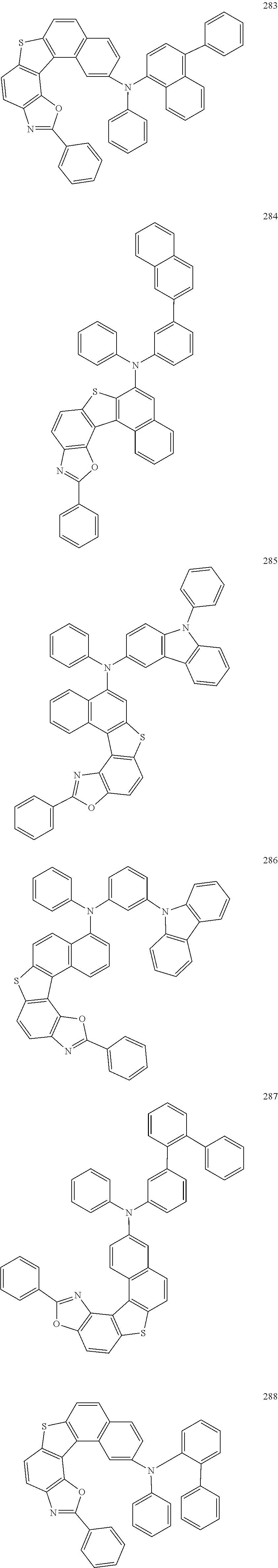 [SMILES: c1ccc(-c2nc3ccc4sc5ccc6ccc(N(c7ccccc7)c7ccc(-c8ccccc8)c8ccccc78)cc6c5c4c3o2)cc1 ; c1ccc(-c2nc3ccc4sc5c(N(c6ccccc6)c6cccc(-c7ccc8ccccc8c7)c6)cc6ccccc6c5c4c3o2)cc1 ; c1ccc(-c2nc3c(ccc4sc5cc(N(c6ccccc6)c6ccc7c(c6)c6ccccc6n7-c6ccccc6)c6ccccc6c5c43)o2)cc1 ; c1ccc(-c2nc3ccc4sc5ccc6c(N(c7ccccc7)c7cccc(-n8c9ccccc9c9ccccc98)c7)cccc6c5c4c3o2)cc1 ; c1ccc(-c2nc3c(ccc4sc5ccc6cc(N(c7ccccc7)c7cccc(-c8ccccc8-c8ccccc8)c7)ccc6c5c43)o2)cc1 ; c1ccc(-c2nc3ccc4sc5ccc6ccc(N(c7ccccc7)c7ccccc7-c7ccccc7)cc6c5c4c3o2)cc1]

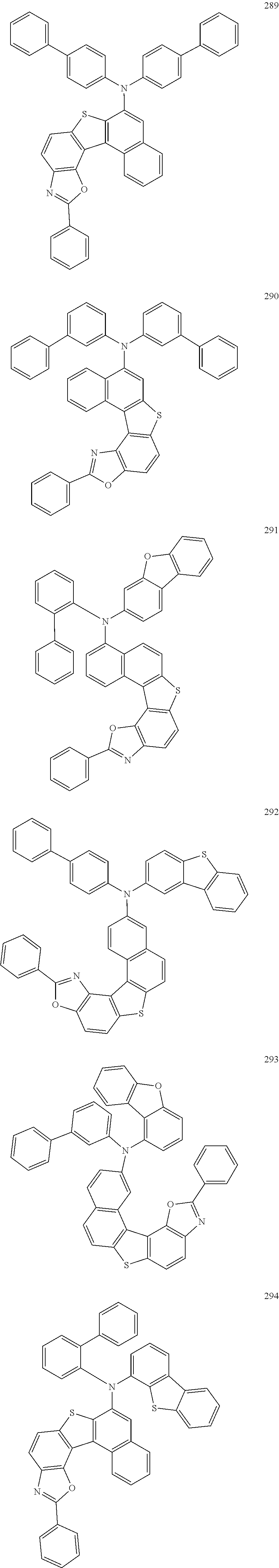

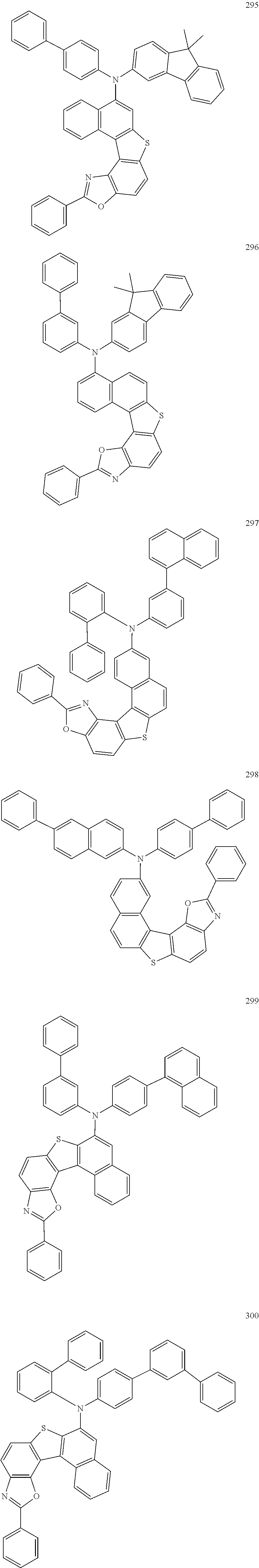

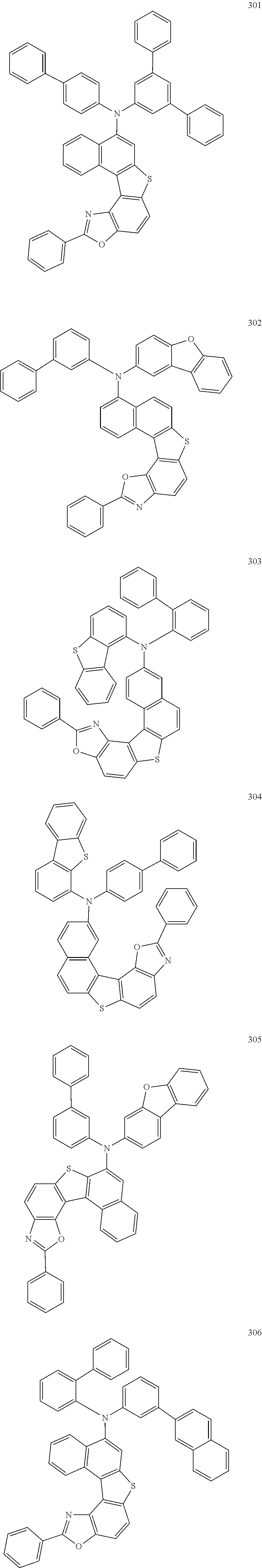

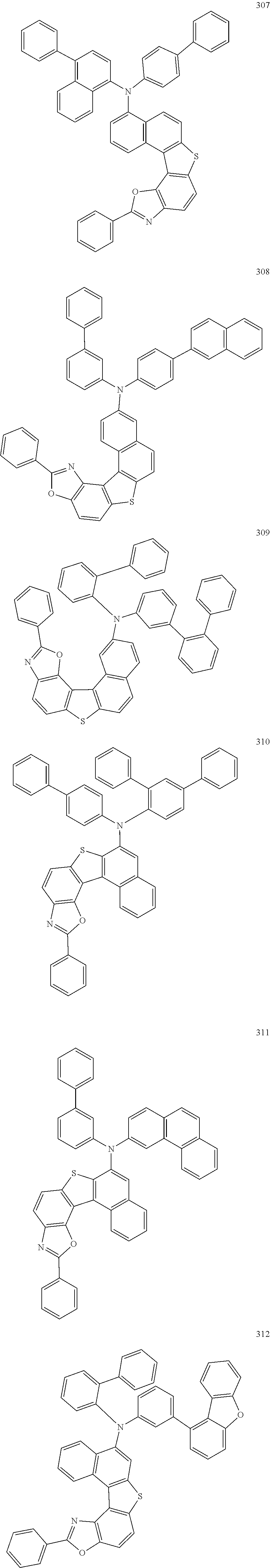

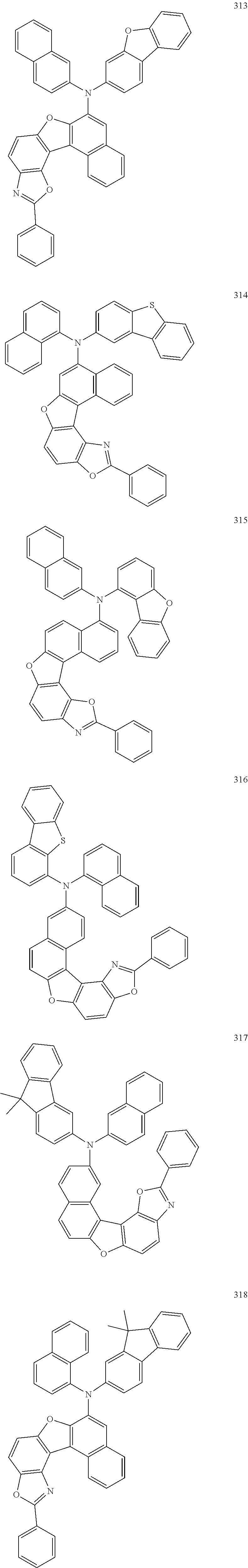

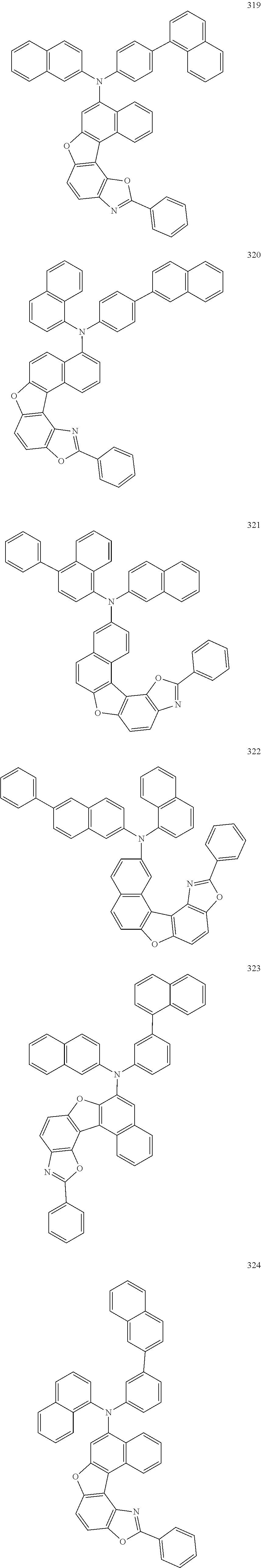

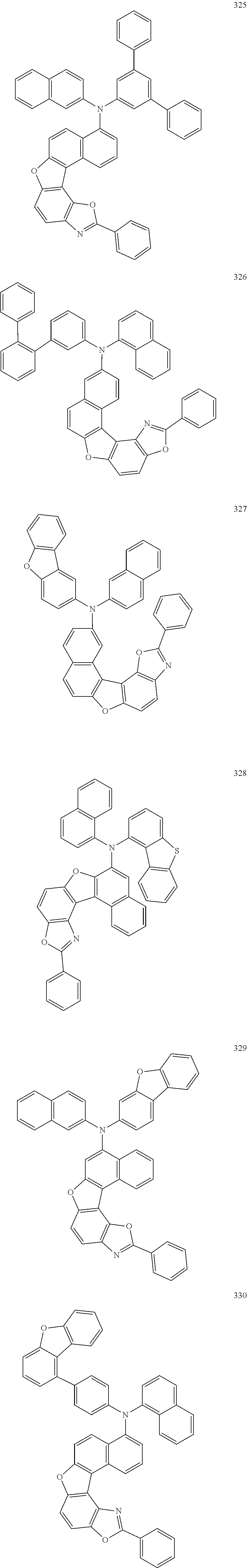

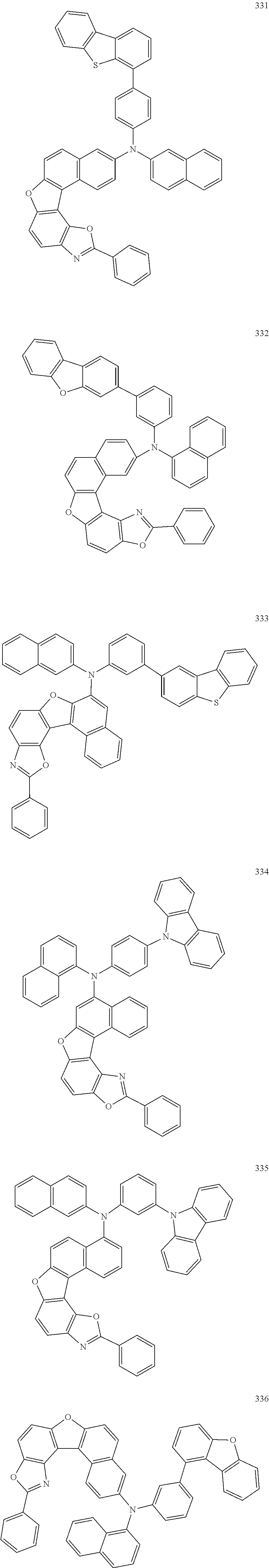

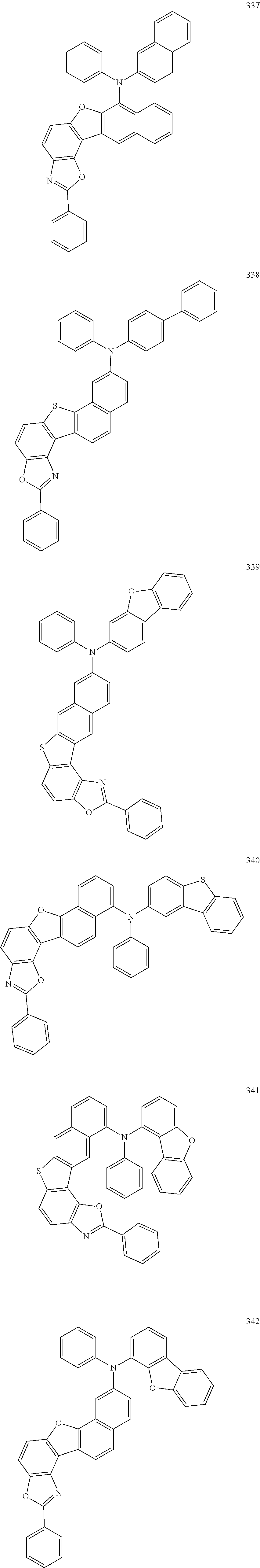 [SMILES: c1ccc(-c2nc3ccc4oc5c(N(c6ccccc6)c6ccc7ccccc7c6)c6ccccc6cc5c4c3o2)cc1 ; c1ccc(-c2ccc(N(c3ccccc3)c3ccc4ccc5c(sc6ccc7oc(-c8ccccc8)nc7c65)c4c3)cc2)cc1 ; c1ccc(-c2nc3c(ccc4sc5cc6cc(N(c7ccccc7)c7ccc8c(c7)oc7ccccc78)ccc6cc5c43)o2)cc1 ; c1ccc(-c2nc3ccc4oc5c6cccc(N(c7ccccc7)c7ccc8sc9ccccc9c8c7)c6ccc5c4c3o2)cc1 ; c1ccc(-c2nc3ccc4sc5cc6cccc(N(c7ccccc7)c7cccc8oc9ccccc9c78)c6cc5c4c3o2)cc1 ; c1ccc(-c2nc3c(ccc4oc5c6cc(N(c7ccccc7)c7cccc8c7oc7ccccc78)ccc6ccc5c43)o2)cc1]

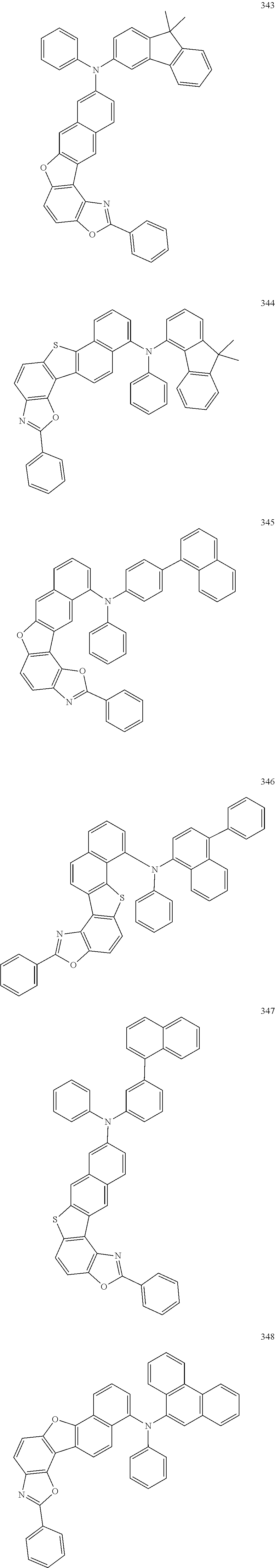 [SMILES: CC1(C)c2ccccc2-c2cc(N(c3ccccc3)c3ccc4cc5c(cc4c3)oc3ccc4oc(-c6ccccc6)nc4c35)ccc21 ; CC1(C)c2ccccc2-c2c(N(c3ccccc3)c3cccc4c3ccc3c4sc4ccc5nc(-c6ccccc6)oc5c43)cccc21 ; c1ccc(-c2nc3ccc4oc5cc6cccc(N(c7ccccc7)c7ccc(-c8cccc9ccccc89)cc7)c6cc5c4c3o2)cc1 ; c1ccc(-c2nc3c(ccc4sc5c(ccc6cccc(N(c7ccccc7)c7ccc(-c8ccccc8)c8ccccc78)c65)c43)o2)cc1 ; c1ccc(-c2nc3c(ccc4sc5cc6cc(N(c7ccccc7)c7cccc(-c8cccc9ccccc89)c7)ccc6cc5c43)o2)cc1 ; c1ccc(-c2nc3ccc4oc5c6cccc(N(c7ccccc7)c7cc8ccccc8c8ccccc78)c6ccc5c4c3o2)cc1]

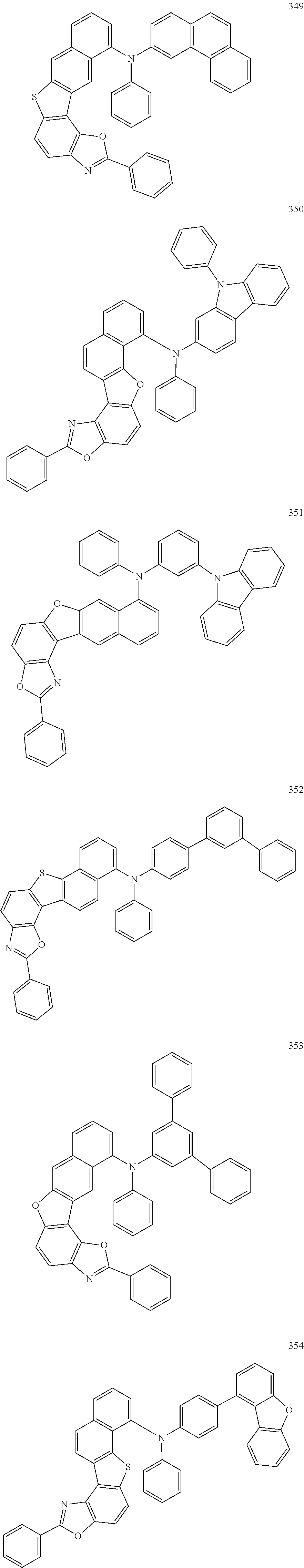

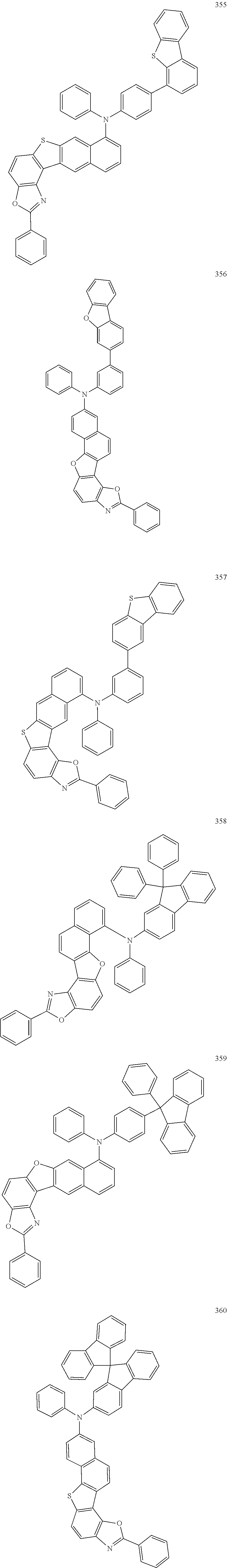

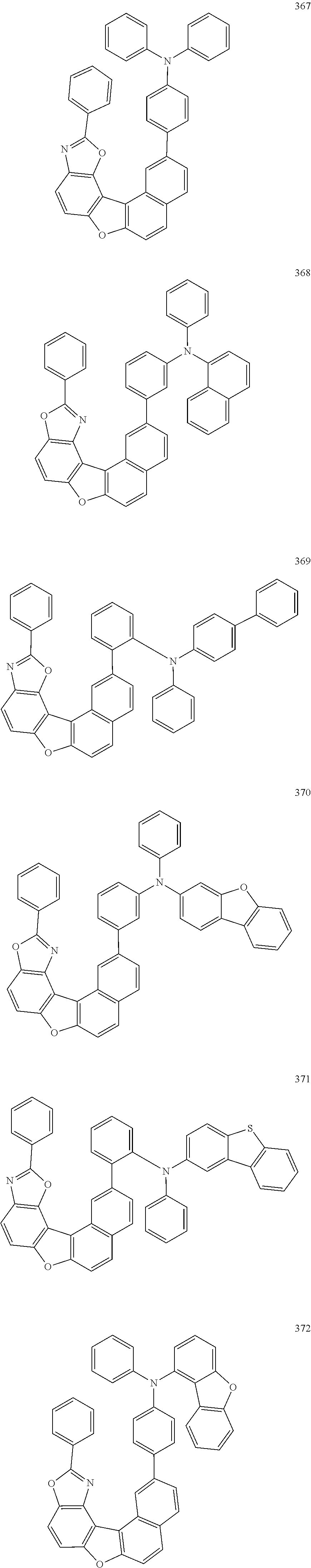

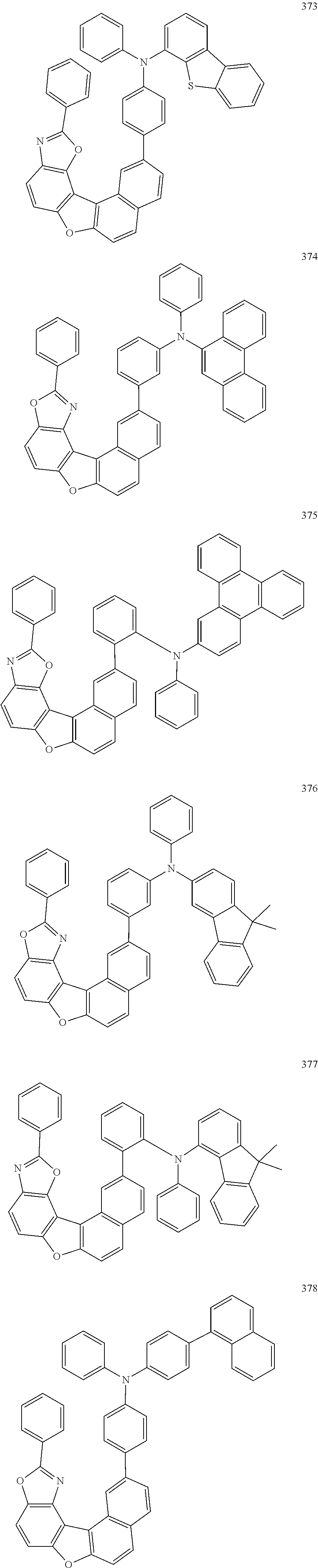

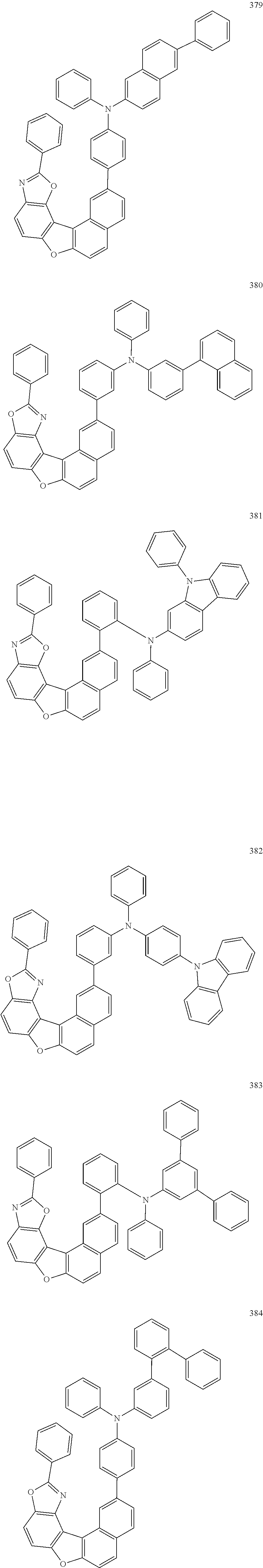

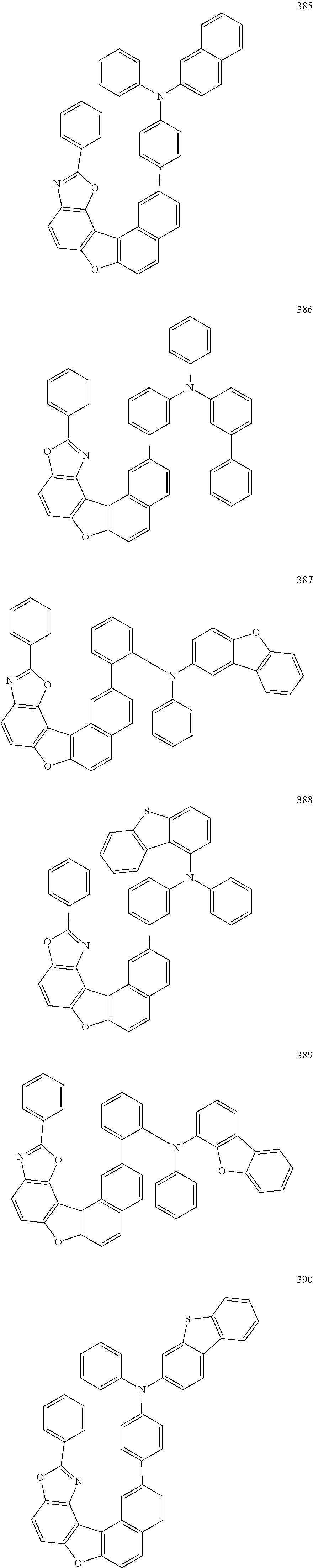

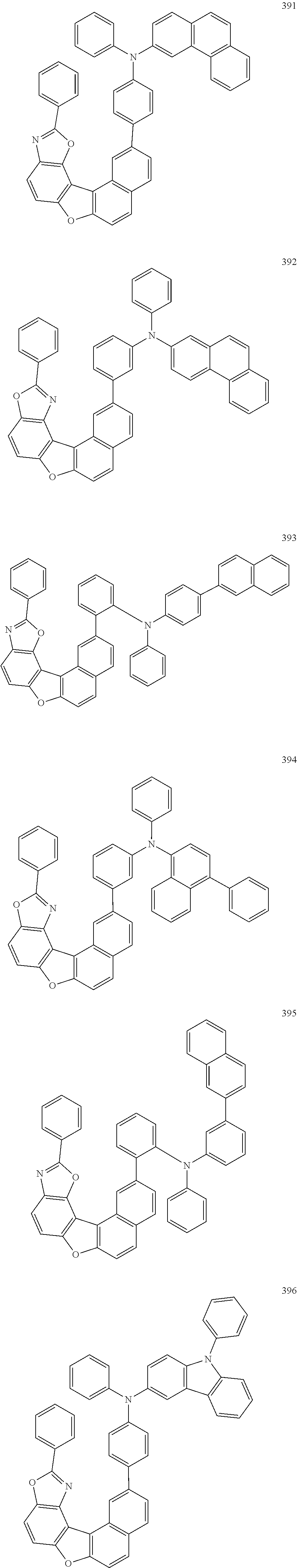

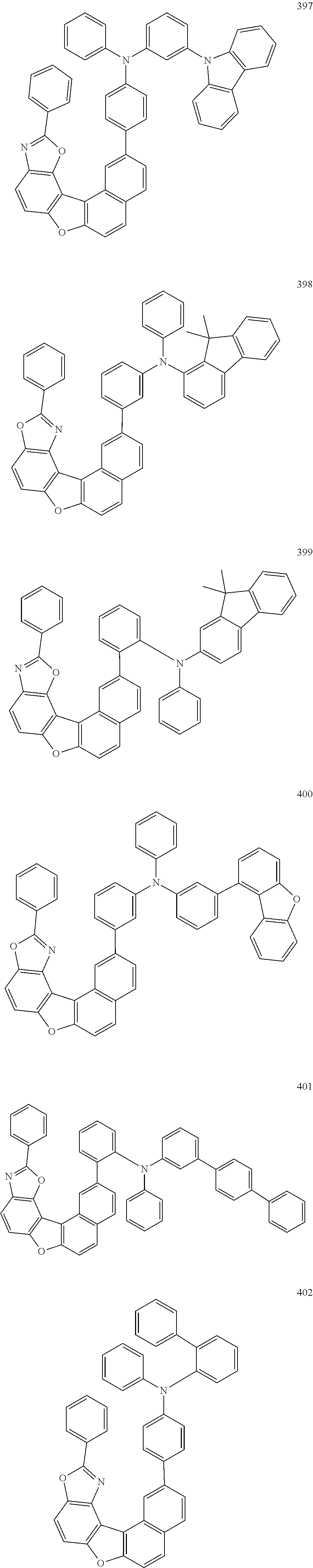

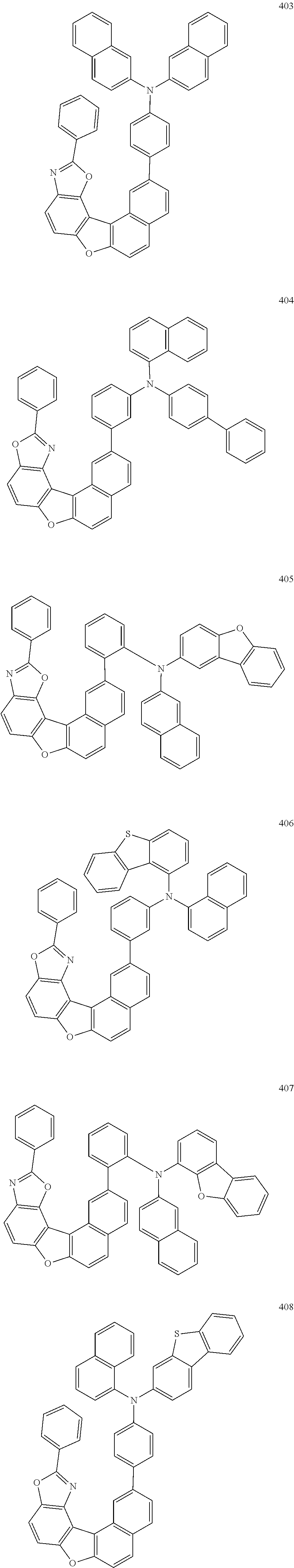

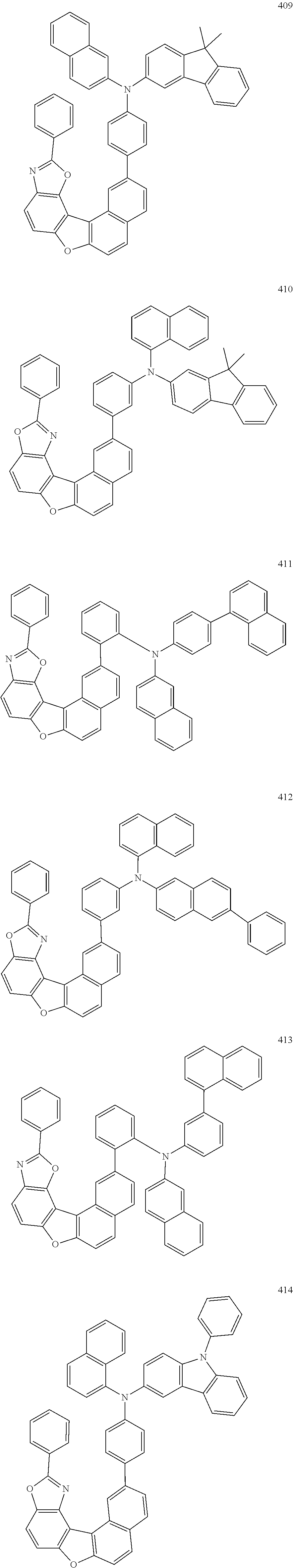

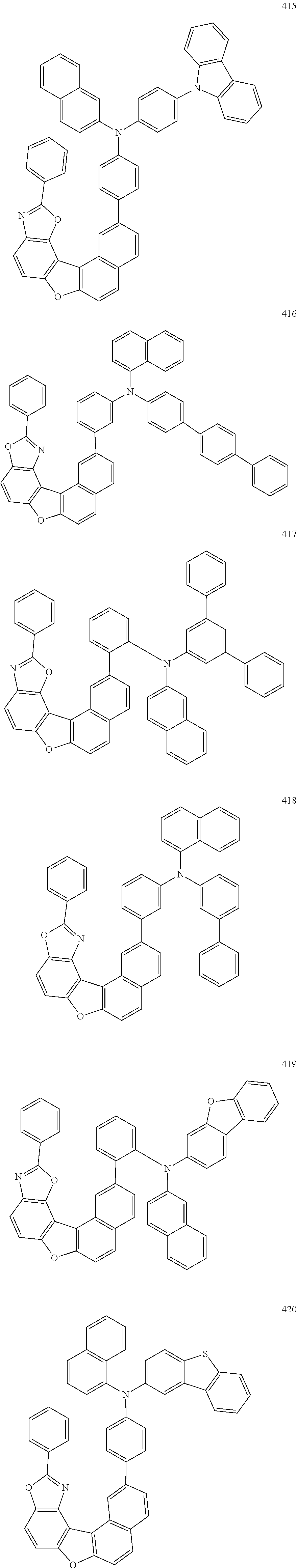

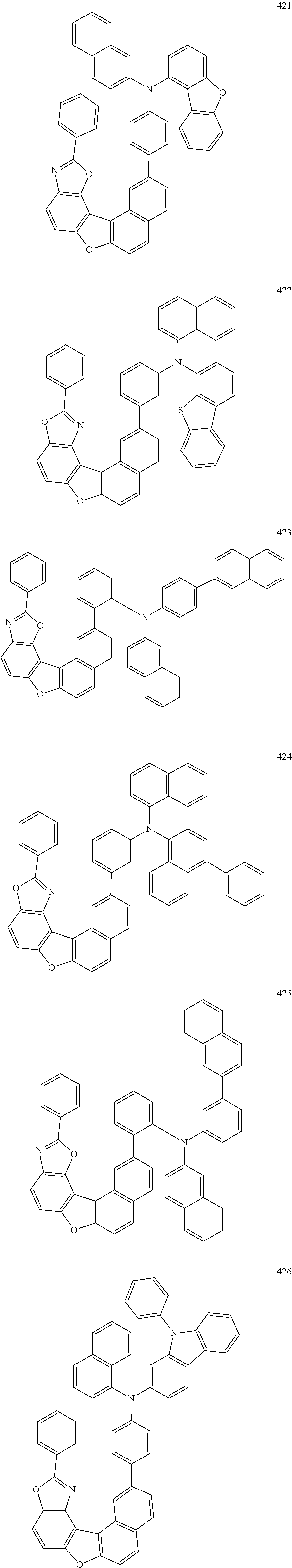

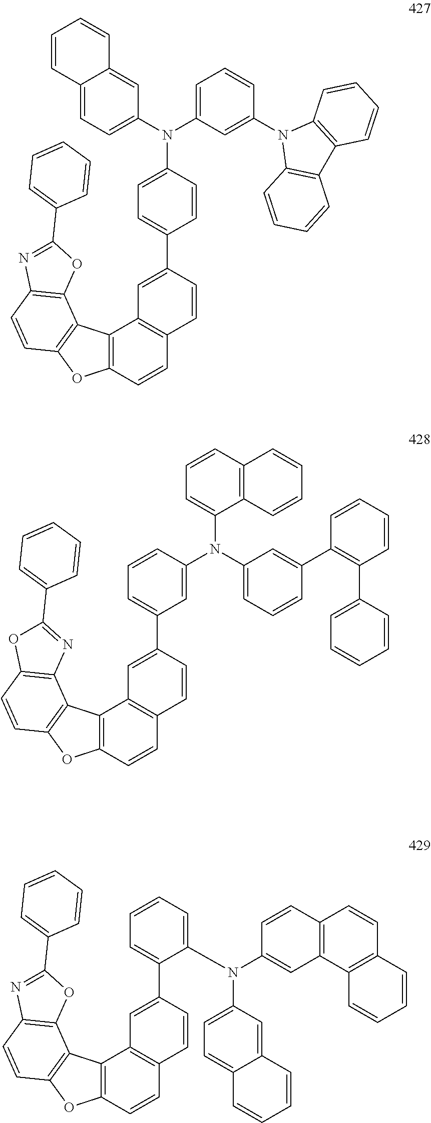 [SMILES: c1ccc(-c2nc3ccc4oc5ccc6ccc(-c7ccc(N(c8cccc(-n9c%10ccccc%10c%10ccccc%109)c8)c8ccc9ccccc9c8)cc7)cc6c5c4c3o2)cc1 ; c1ccc(-c2nc3c(ccc4oc5ccc6ccc(-c7cccc(N(c8cccc(-c9ccccc9-c9ccccc9)c8)c8cccc9ccccc89)c7)cc6c5c43)o2)cc1 ; c1ccc(-c2nc3ccc4oc5ccc6ccc(-c7ccccc7N(c7ccc8ccccc8c7)c7ccc8ccc9ccccc9c8c7)cc6c5c4c3o2)cc1]

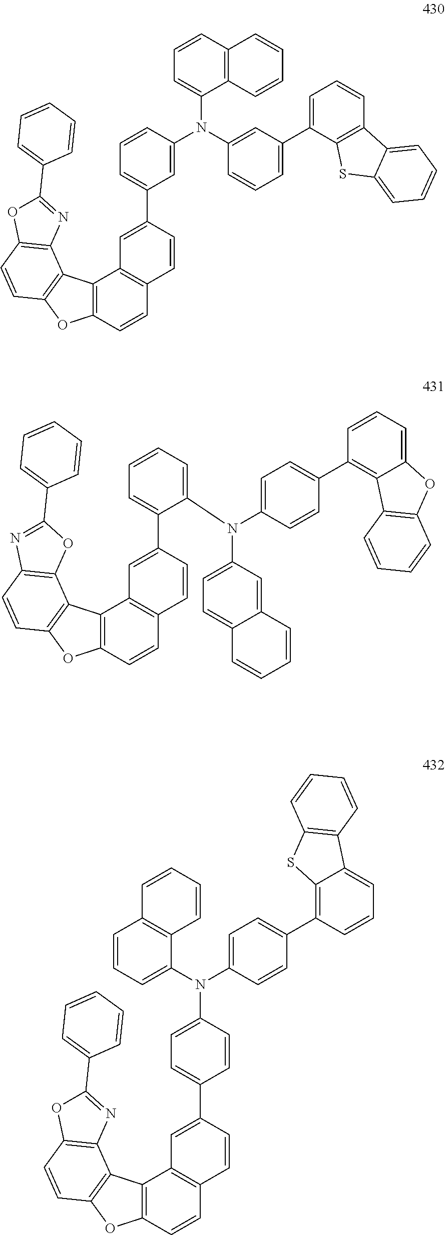 [SMILES: c1ccc(-c2nc3c(ccc4oc5ccc6ccc(-c7cccc(N(c8cccc(-c9cccc%10c9sc9ccccc9%10)c8)c8cccc9ccccc89)c7)cc6c5c43)o2)cc1 ; c1ccc(-c2nc3ccc4oc5ccc6ccc(-c7ccccc7N(c7ccc(-c8cccc9oc%10ccccc%10c89)cc7)c7ccc8ccccc8c7)cc6c5c4c3o2)cc1 ; c1ccc(-c2nc3c(ccc4oc5ccc6ccc(-c7ccc(N(c8ccc(-c9cccc%10c9sc9ccccc9%10)cc8)c8cccc9ccccc89)cc7)cc6c5c43)o2)cc1]

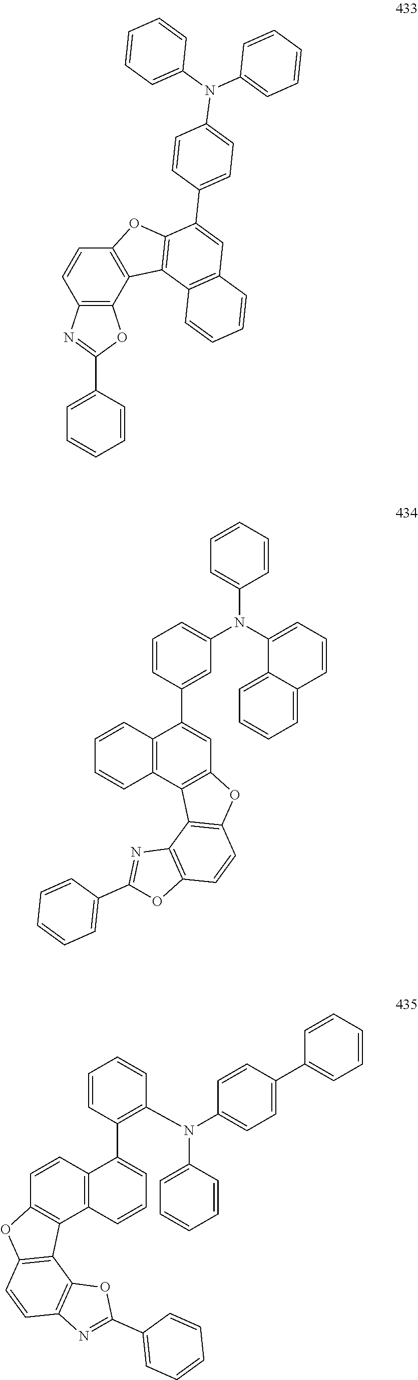 [SMILES: c1ccc(-c2nc3ccc4oc5c(-c6ccc(N(c7ccccc7)c7ccccc7)cc6)cc6ccccc6c5c4c3o2)cc1 ; c1ccc(-c2nc3c(ccc4oc5cc(-c6cccc(N(c7ccccc7)c7cccc8ccccc78)c6)c6ccccc6c5c43)o2)cc1 ; c1ccc(-c2ccc(N(c3ccccc3)c3ccccc3-c3cccc4c3ccc3oc5ccc6nc(-c7ccccc7)oc6c5c34)cc2)cc1]

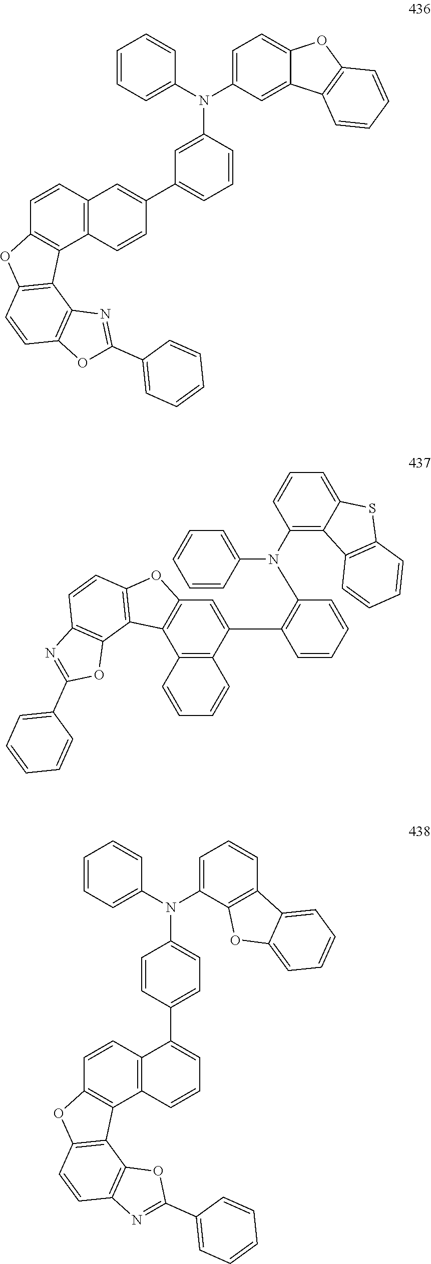 [SMILES: c1ccc(-c2nc3c(ccc4oc5ccc6cc(-c7cccc(N(c8ccccc8)c8ccc9oc%10ccccc%10c9c8)c7)ccc6c5c43)o2)cc1 ; c1ccc(-c2nc3ccc4oc5cc(-c6ccccc6N(c6ccccc6)c6cccc7sc8ccccc8c67)c6ccccc6c5c4c3o2)cc1 ; c1ccc(-c2nc3ccc4oc5ccc6c(-c7ccc(N(c8ccccc8)c8cccc9c8oc8ccccc89)cc7)cccc6c5c4c3o2)cc1]

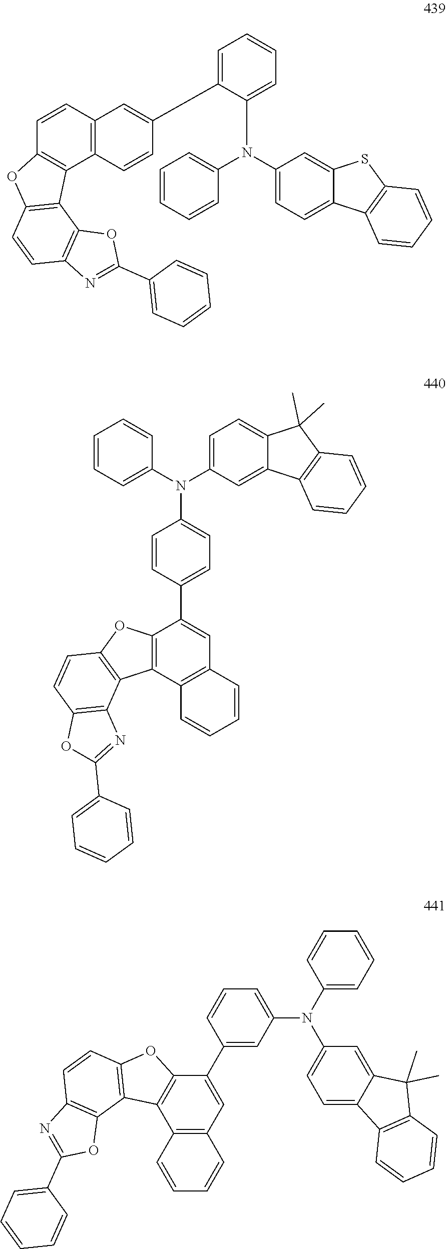 [SMILES: c1ccc(-c2nc3ccc4oc5ccc6cc(-c7ccccc7N(c7ccccc7)c7ccc8c(c7)sc7ccccc78)ccc6c5c4c3o2)cc1 ; CC1(C)c2ccccc2-c2cc(N(c3ccccc3)c3ccc(-c4cc5ccccc5c5c4oc4ccc6oc(-c7ccccc7)nc6c45)cc3)ccc21 ; CC1(C)c2ccccc2-c2ccc(N(c3ccccc3)c3cccc(-c4cc5ccccc5c5c4oc4ccc6nc(-c7ccccc7)oc6c45)c3)cc21]

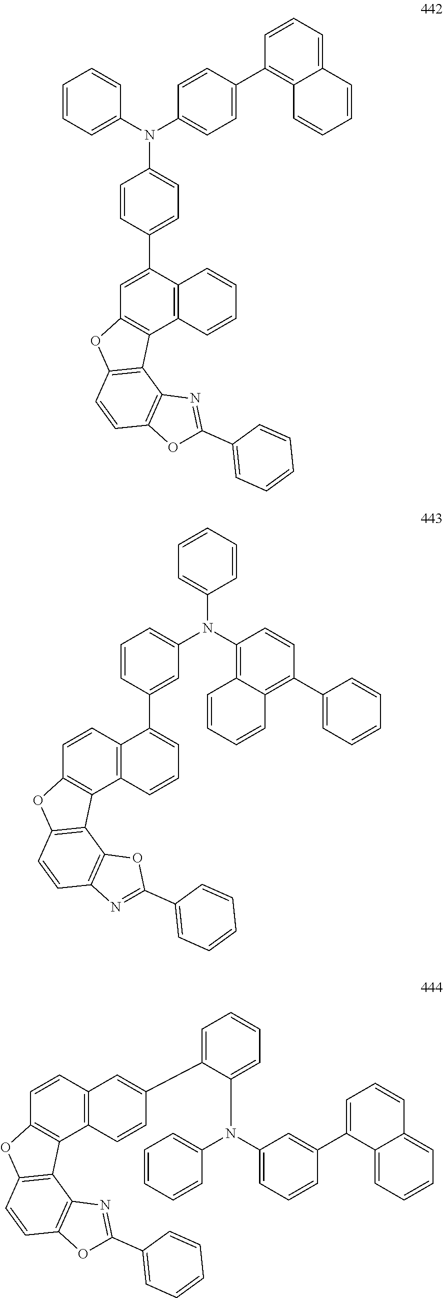 [SMILES: c1ccc(-c2nc3c(ccc4oc5cc(-c6ccc(N(c7ccccc7)c7ccc(-c8cccc9ccccc89)cc7)cc6)c6ccccc6c5c43)o2)cc1 ; c1ccc(-c2nc3ccc4oc5ccc6c(-c7cccc(N(c8ccccc8)c8ccc(-c9ccccc9)c9ccccc89)c7)cccc6c5c4c3o2)cc1 ; c1ccc(-c2nc3c(ccc4oc5ccc6cc(-c7ccccc7N(c7ccccc7)c7cccc(-c8cccc9ccccc89)c7)ccc6c5c43)o2)cc1]

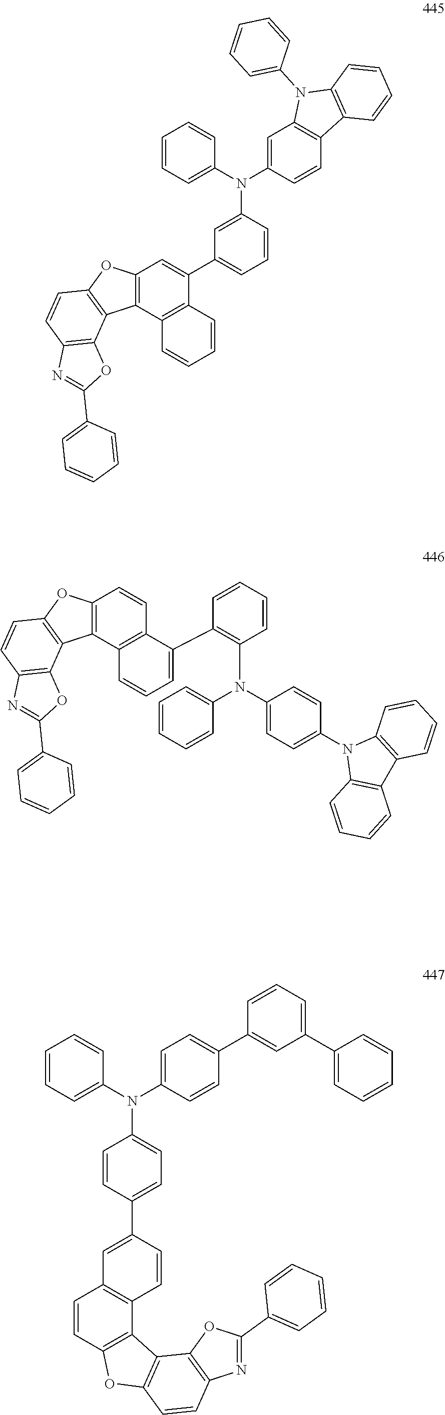 [SMILES: c1ccc(-c2nc3ccc4oc5cc(-c6cccc(N(c7ccccc7)c7ccc8c9ccccc9n(-c9ccccc9)c8c7)c6)c6ccccc6c5c4c3o2)cc1 ; c1ccc(-c2nc3ccc4oc5ccc6c(-c7ccccc7N(c7ccccc7)c7ccc(-n8c9ccccc9c9ccccc98)cc7)cccc6c5c4c3o2)cc1 ; c1ccc(-c2cccc(-c3ccc(N(c4ccccc4)c4ccc(-c5ccc6c(ccc7oc8ccc9nc(-c%10ccccc%10)oc9c8c76)c5)cc4)cc3)c2)cc1]

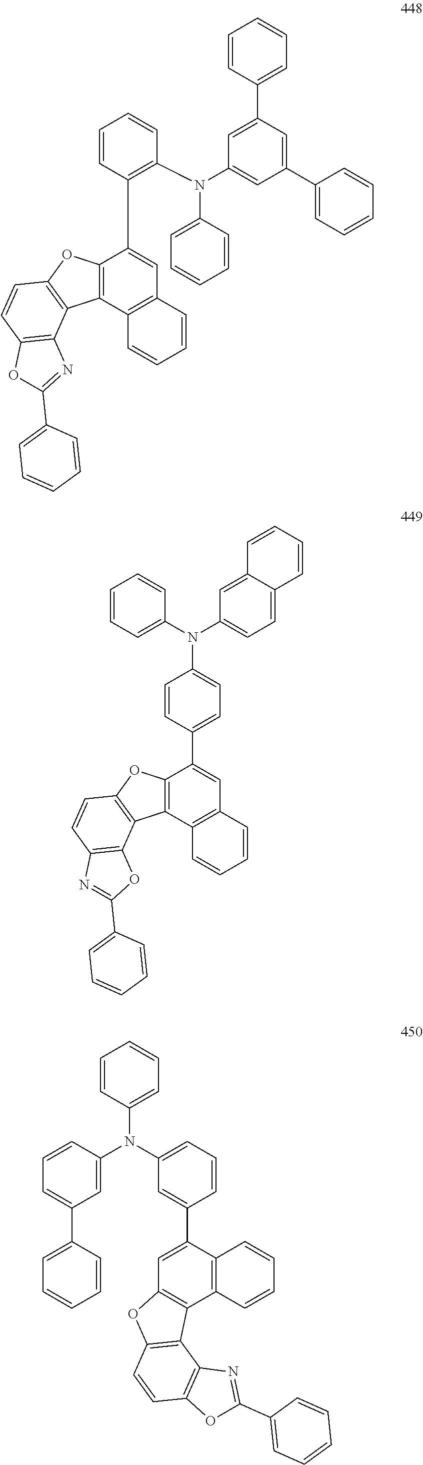 [SMILES: c1ccc(-c2cc(-c3ccccc3)cc(N(c3ccccc3)c3ccccc3-c3cc4ccccc4c4c3oc3ccc5oc(-c6ccccc6)nc5c34)c2)cc1 ; c1ccc(-c2nc3ccc4oc5c(-c6ccc(N(c7ccccc7)c7ccc8ccccc8c7)cc6)cc6ccccc6c5c4c3o2)cc1 ; c1ccc(-c2cccc(N(c3ccccc3)c3cccc(-c4cc5oc6ccc7oc(-c8ccccc8)nc7c6c5c5ccccc45)c3)c2)cc1]

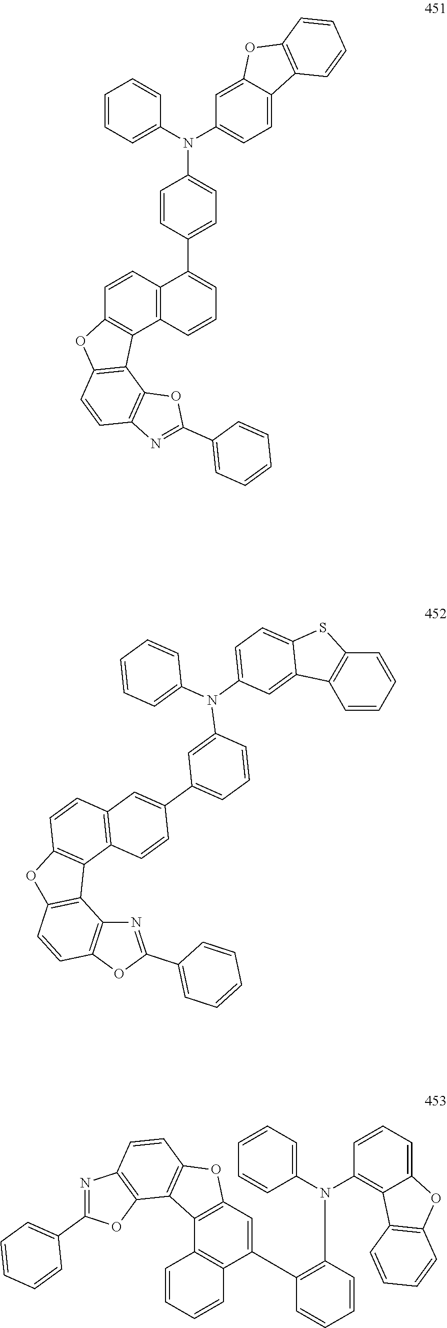 [SMILES: c1ccc(-c2nc3ccc4oc5ccc6c(-c7ccc(N(c8ccccc8)c8ccc9c(c8)oc8ccccc89)cc7)cccc6c5c4c3o2)cc1 ; c1ccc(-c2nc3c(ccc4oc5ccc6cc(-c7cccc(N(c8ccccc8)c8ccc9sc%10ccccc%10c9c8)c7)ccc6c5c43)o2)cc1 ; c1ccc(-c2nc3ccc4oc5cc(-c6ccccc6N(c6ccccc6)c6cccc7oc8ccccc8c67)c6ccccc6c5c4c3o2)cc1]

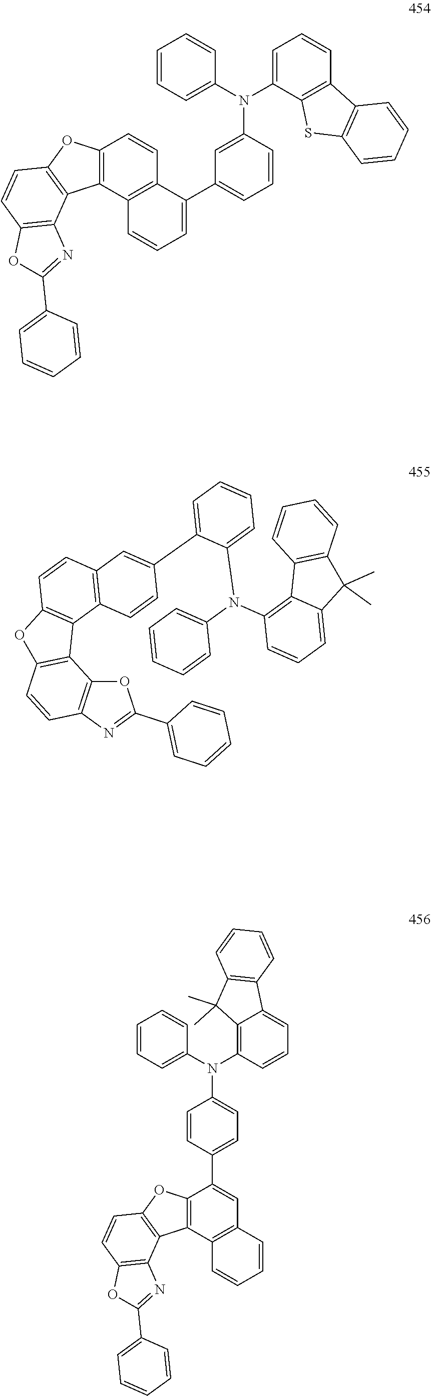 [SMILES: c1ccc(-c2nc3c(ccc4oc5ccc6c(-c7cccc(N(c8ccccc8)c8cccc9c8sc8ccccc89)c7)cccc6c5c43)o2)cc1 ; CC1(C)c2ccccc2-c2c(N(c3ccccc3)c3ccccc3-c3ccc4c(ccc5oc6ccc7nc(-c8ccccc8)oc7c6c54)c3)cccc21 ; CC1(C)c2ccccc2-c2cccc(N(c3ccccc3)c3ccc(-c4cc5ccccc5c5c4oc4ccc6oc(-c7ccccc7)nc6c45)cc3)c21]

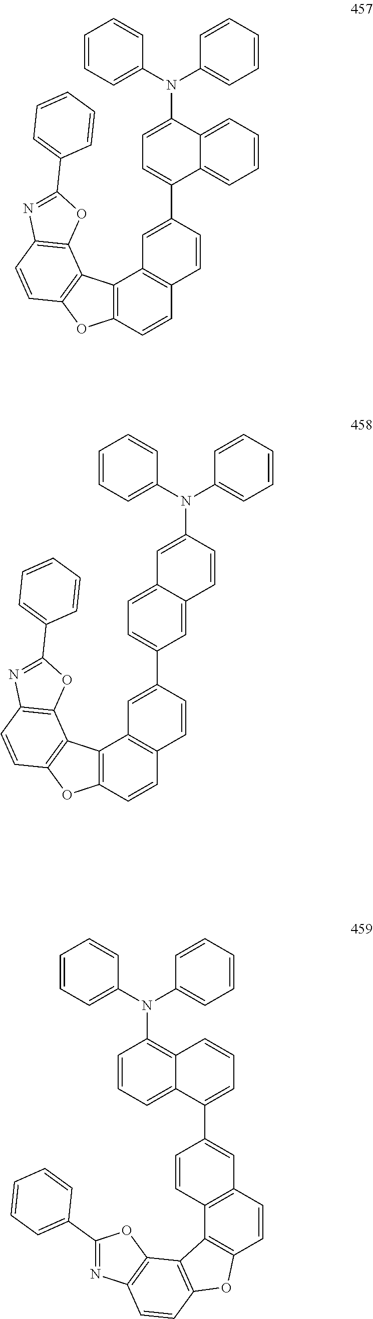 [SMILES: c1ccc(-c2nc3ccc4oc5ccc6ccc(-c7ccc(N(c8ccccc8)c8ccccc8)c8ccccc78)cc6c5c4c3o2)cc1 ; c1ccc(-c2nc3ccc4oc5ccc6ccc(-c7ccc8cc(N(c9ccccc9)c9ccccc9)ccc8c7)cc6c5c4c3o2)cc1 ; c1ccc(-c2nc3ccc4oc5ccc6cc(-c7cccc8c(N(c9ccccc9)c9ccccc9)cccc78)ccc6c5c4c3o2)cc1]

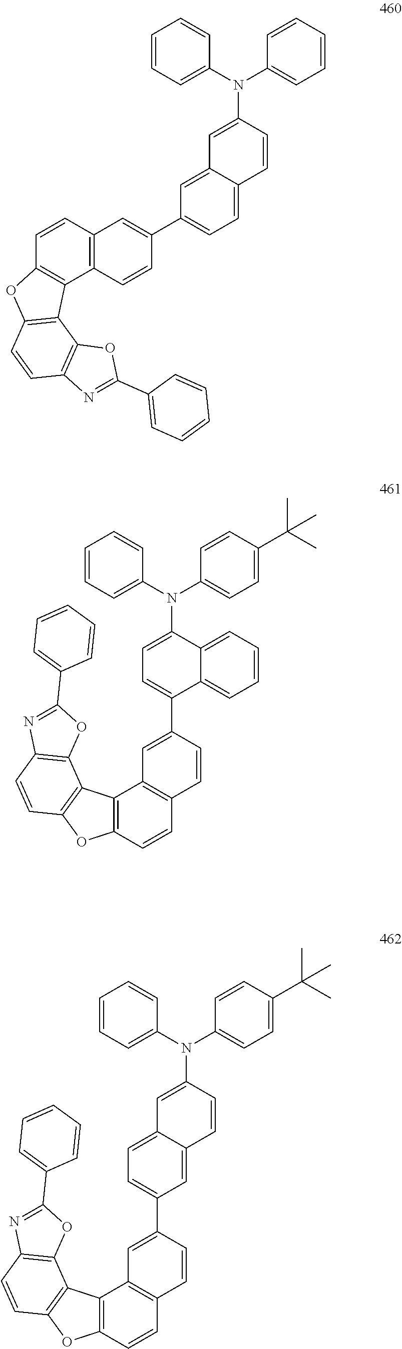 [SMILES: c1ccc(-c2nc3ccc4oc5ccc6cc(-c7ccc8ccc(N(c9ccccc9)c9ccccc9)cc8c7)ccc6c5c4c3o2)cc1 ; CC(C)(C)c1ccc(N(c2ccccc2)c2ccc(-c3ccc4ccc5oc6ccc7nc(-c8ccccc8)oc7c6c5c4c3)c3ccccc23)cc1 ; CC(C)(C)c1ccc(N(c2ccccc2)c2ccc3cc(-c4ccc5ccc6oc7ccc8nc(-c9ccccc9)oc8c7c6c5c4)ccc3c2)cc1]

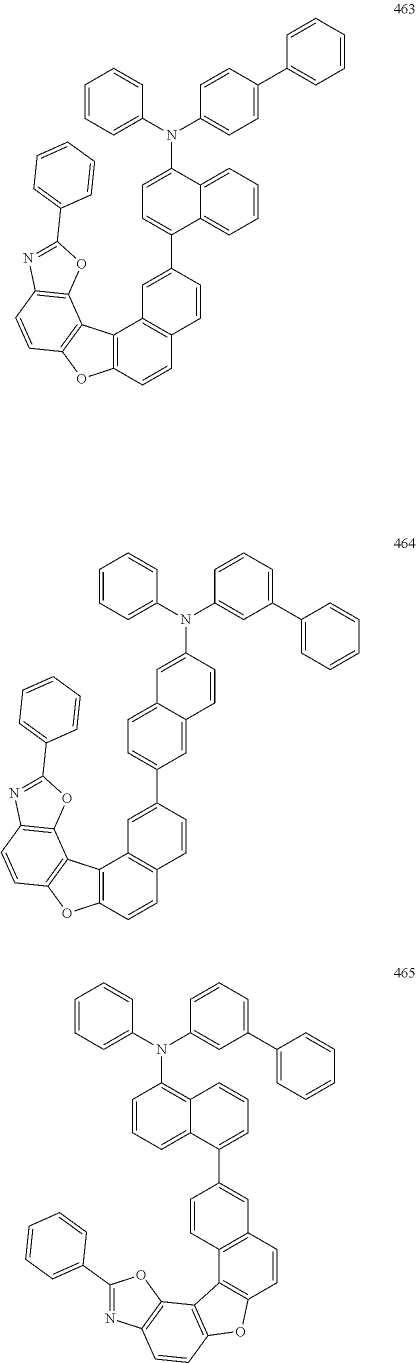 [SMILES: c1ccc(-c2ccc(N(c3ccccc3)c3ccc(-c4ccc5ccc6oc7ccc8nc(-c9ccccc9)oc8c7c6c5c4)c4ccccc34)cc2)cc1 ; c1ccc(-c2cccc(N(c3ccccc3)c3ccc4cc(-c5ccc6ccc7oc8ccc9nc(-c%10ccccc%10)oc9c8c7c6c5)ccc4c3)c2)cc1 ; c1ccc(-c2cccc(N(c3ccccc3)c3cccc4c(-c5ccc6c(ccc7oc8ccc9nc(-c%10ccccc%10)oc9c8c76)c5)cccc34)c2)cc1]

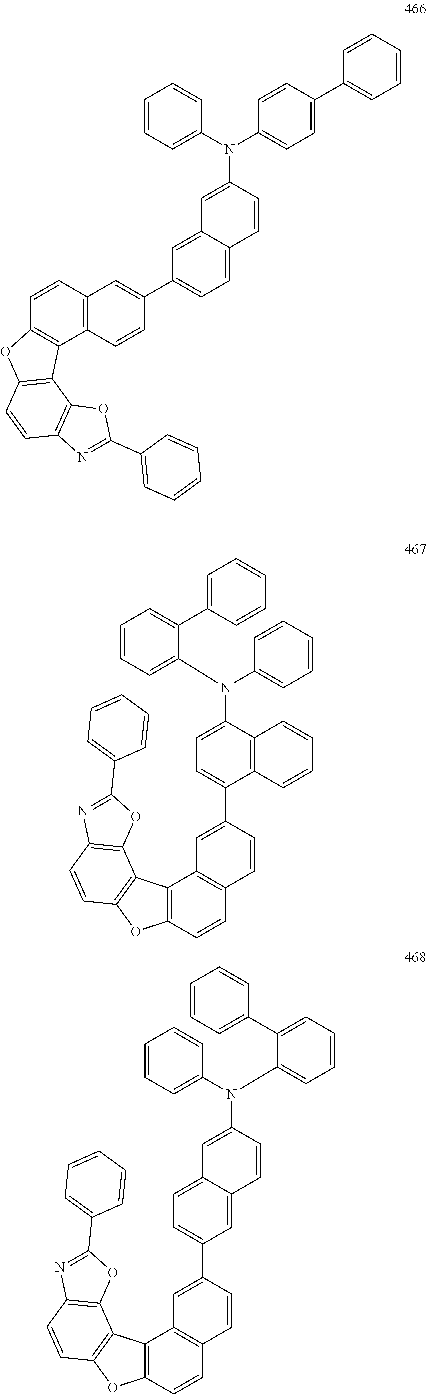

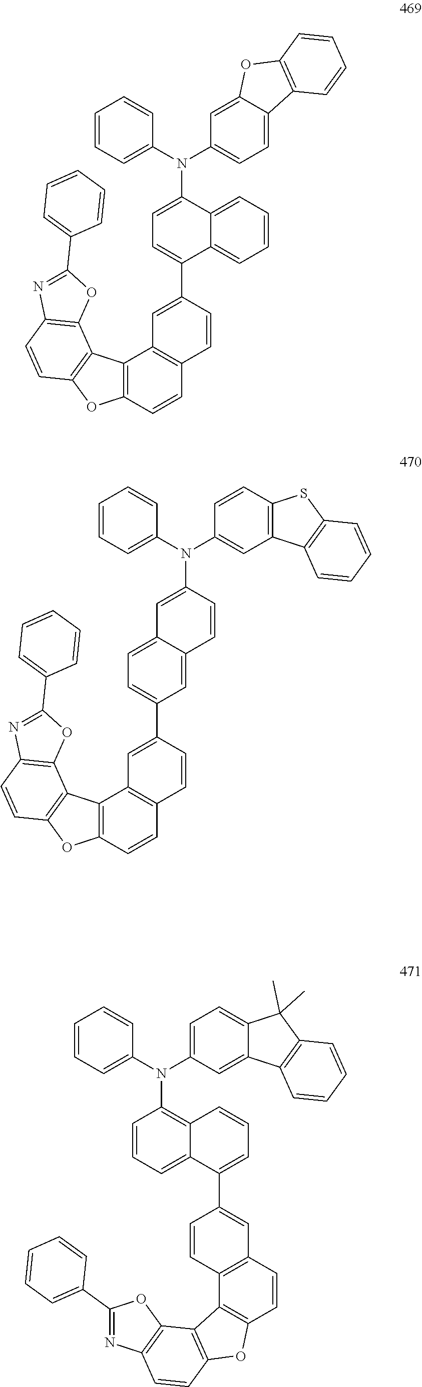 [SMILES: c1ccc(-c2nc3ccc4oc5ccc6ccc(-c7ccc(N(c8ccccc8)c8ccc9c(c8)oc8ccccc89)c8ccccc78)cc6c5c4c3o2)cc1 ; c1ccc(-c2nc3ccc4oc5ccc6ccc(-c7ccc8cc(N(c9ccccc9)c9ccc%10sc%11ccccc%11c%10c9)ccc8c7)cc6c5c4c3o2)cc1 ; CC1(C)c2ccccc2-c2cc(N(c3ccccc3)c3cccc4c(-c5ccc6c(ccc7oc8ccc9nc(-c%10ccccc%10)oc9c8c76)c5)cccc34)ccc21]

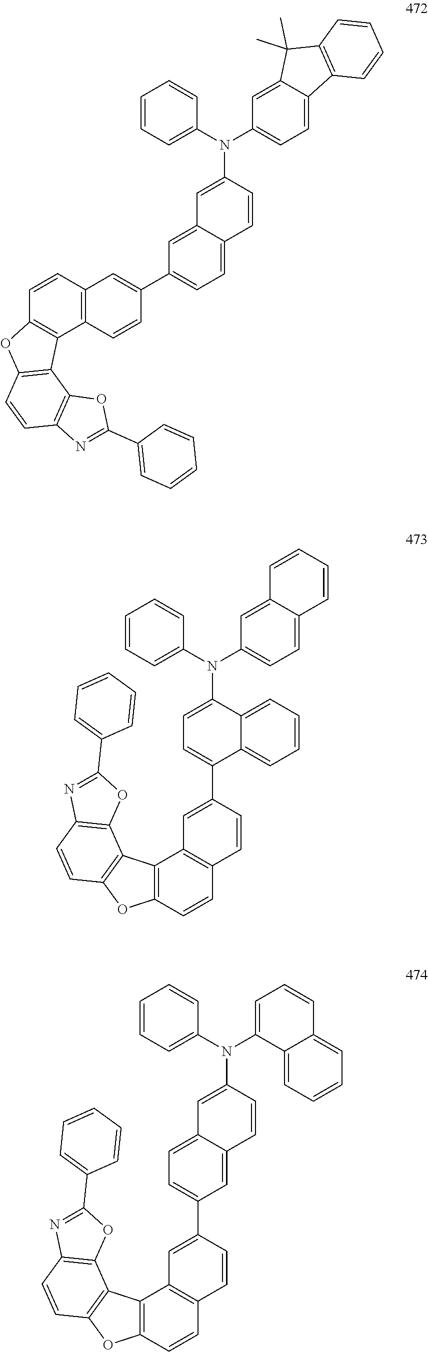 [SMILES: CC1(C)c2ccccc2-c2ccc(N(c3ccccc3)c3ccc4ccc(-c5ccc6c(ccc7oc8ccc9nc(-c%10ccccc%10)oc9c8c76)c5)cc4c3)cc21 ; c1ccc(-c2nc3ccc4oc5ccc6ccc(-c7ccc(N(c8ccccc8)c8ccc9ccccc9c8)c8ccccc78)cc6c5c4c3o2)cc1 ; c1ccc(-c2nc3ccc4oc5ccc6ccc(-c7ccc8cc(N(c9ccccc9)c9cccc%10ccccc9%10)ccc8c7)cc6c5c4c3o2)cc1]

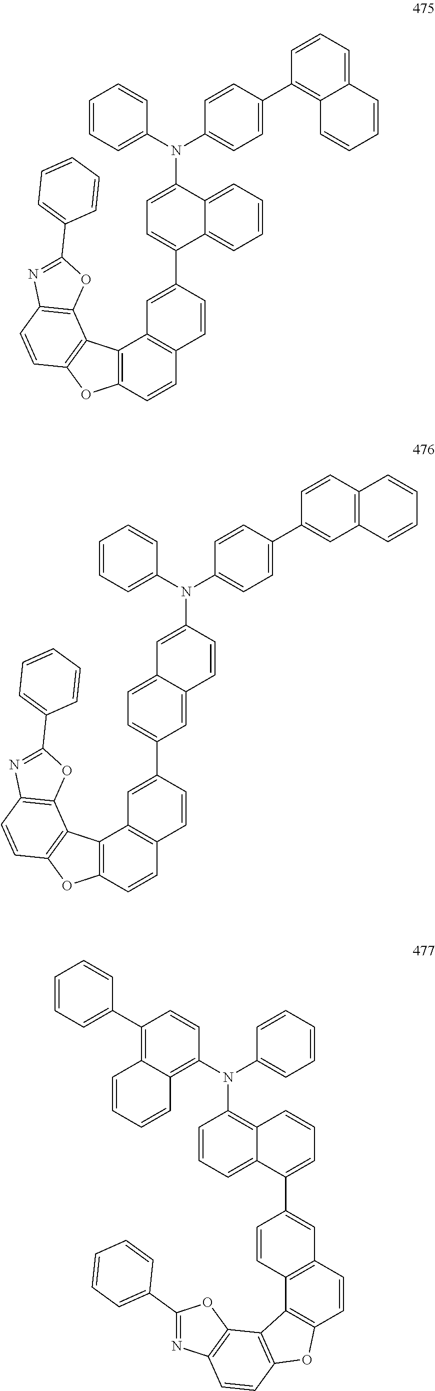 [SMILES: c1ccc(-c2nc3ccc4oc5ccc6ccc(-c7ccc(N(c8ccccc8)c8ccc(-c9cccc%10ccccc9%10)cc8)c8ccccc78)cc6c5c4c3o2)cc1 ; c1ccc(-c2nc3ccc4oc5ccc6ccc(-c7ccc8cc(N(c9ccccc9)c9ccc(-c%10ccc%11ccccc%11c%10)cc9)ccc8c7)cc6c5c4c3o2)cc1 ; c1ccc(-c2nc3ccc4oc5ccc6cc(-c7cccc8c(N(c9ccccc9)c9ccc(-c%10ccccc%10)c%10ccccc9%10)cccc78)ccc6c5c4c3o2)cc1]

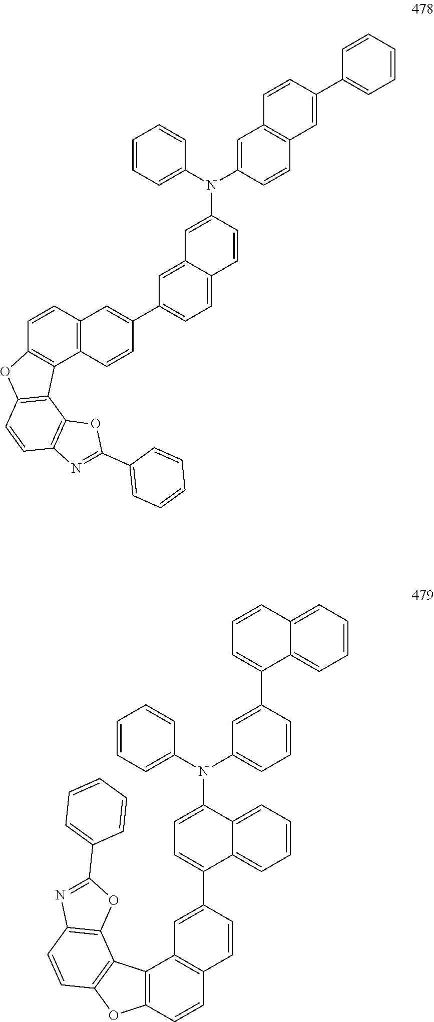 [SMILES: c1ccc(-c2ccc3cc(N(c4ccccc4)c4ccc5ccc(-c6ccc7c(ccc8oc9ccc%10nc(-c%11ccccc%11)oc%10c9c87)c6)cc5c4)ccc3c2)cc1 ; c1ccc(-c2nc3ccc4oc5ccc6ccc(-c7ccc(N(c8ccccc8)c8cccc(-c9cccc%10ccccc9%10)c8)c8ccccc78)cc6c5c4c3o2)cc1]

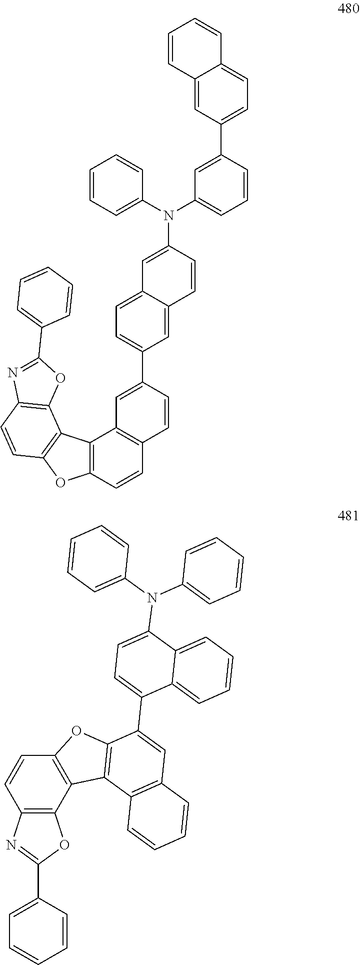

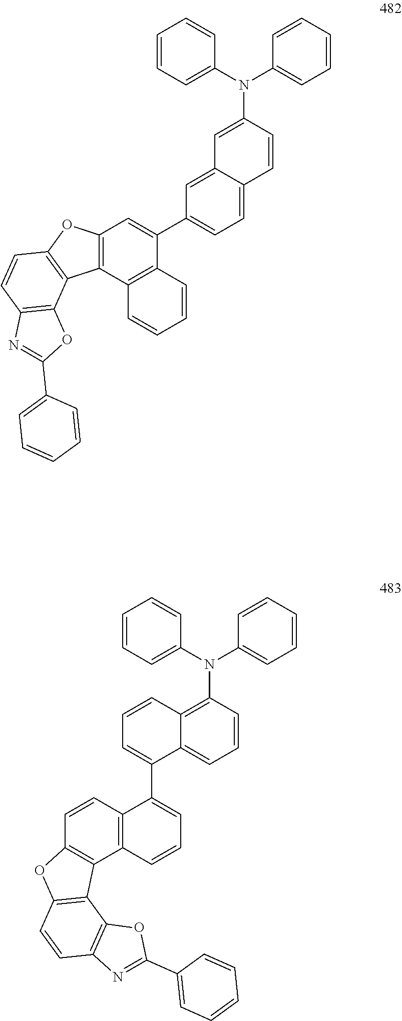 [SMILES: c1ccc(-c2nc3ccc4oc5cc(-c6ccc7ccc(N(c8ccccc8)c8ccccc8)cc7c6)c6ccccc6c5c4c3o2)cc1 ; c1ccc(-c2nc3ccc4oc5ccc6c(-c7cccc8c(N(c9ccccc9)c9ccccc9)cccc78)cccc6c5c4c3o2)cc1]

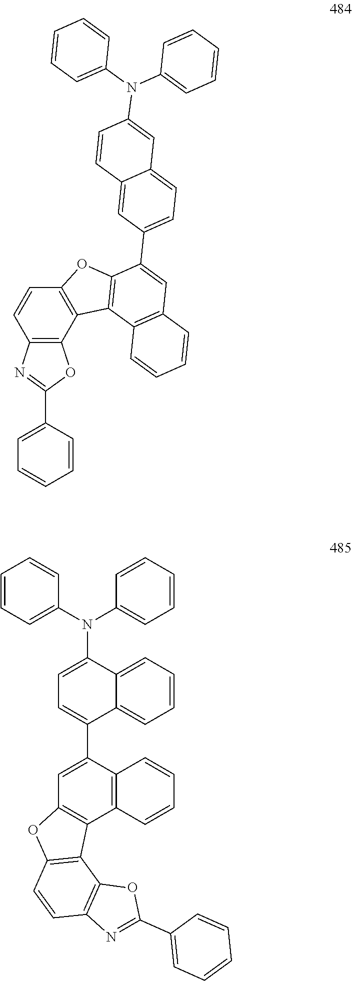 [SMILES: c1ccc(-c2nc3ccc4oc5c(-c6ccc7cc(N(c8ccccc8)c8ccccc8)ccc7c6)cc6ccccc6c5c4c3o2)cc1 ; c1ccc(-c2nc3ccc4oc5cc(-c6ccc(N(c7ccccc7)c7ccccc7)c7ccccc67)c6ccccc6c5c4c3o2)cc1]

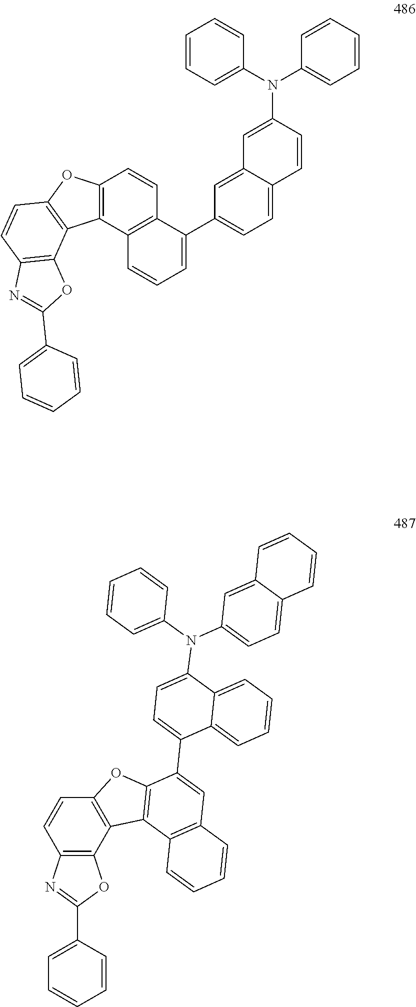 [SMILES: c1ccc(-c2nc3ccc4oc5ccc6c(-c7ccc8ccc(N(c9ccccc9)c9ccccc9)cc8c7)cccc6c5c4c3o2)cc1 ; c1ccc(-c2nc3ccc4oc5c(-c6ccc(N(c7ccccc7)c7ccc8ccccc8c7)c7ccccc67)cc6ccccc6c5c4c3o2)cc1]

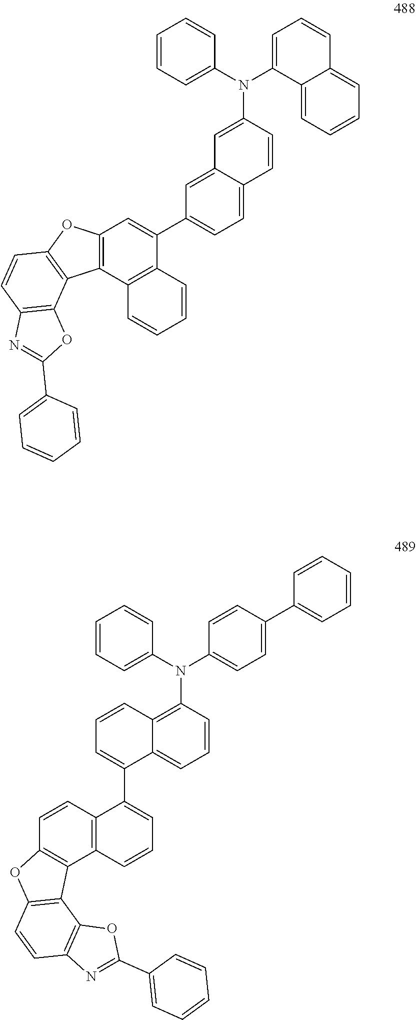 [SMILES: c1ccc(-c2nc3ccc4oc5cc(-c6ccc7ccc(N(c8ccccc8)c8cccc9ccccc89)cc7c6)c6ccccc6c5c4c3o2)cc1 ; c1ccc(-c2ccc(N(c3ccccc3)c3cccc4c(-c5cccc6c5ccc5oc7ccc8nc(-c9ccccc9)oc8c7c56)cccc34)cc2)cc1]

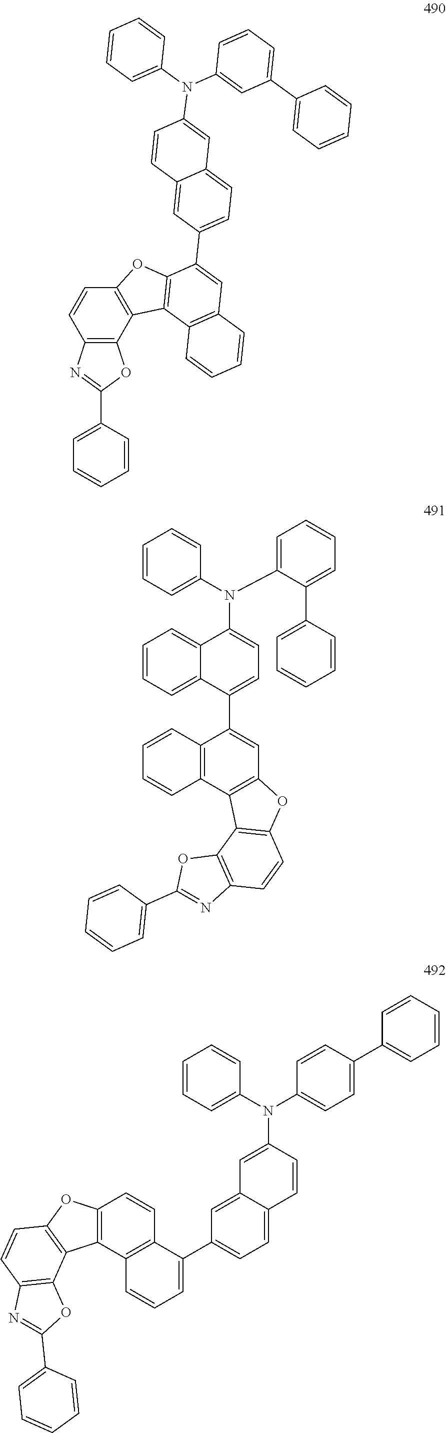 [SMILES: c1ccc(-c2cccc(N(c3ccccc3)c3ccc4cc(-c5cc6ccccc6c6c5oc5ccc7nc(-c8ccccc8)oc7c56)ccc4c3)c2)cc1 ; c1ccc(-c2nc3ccc4oc5cc(-c6ccc(N(c7ccccc7)c7ccccc7-c7ccccc7)c7ccccc67)c6ccccc6c5c4c3o2)cc1 ; c1ccc(-c2ccc(N(c3ccccc3)c3ccc4ccc(-c5cccc6c5ccc5oc7ccc8nc(-c9ccccc9)oc8c7c56)cc4c3)cc2)cc1]

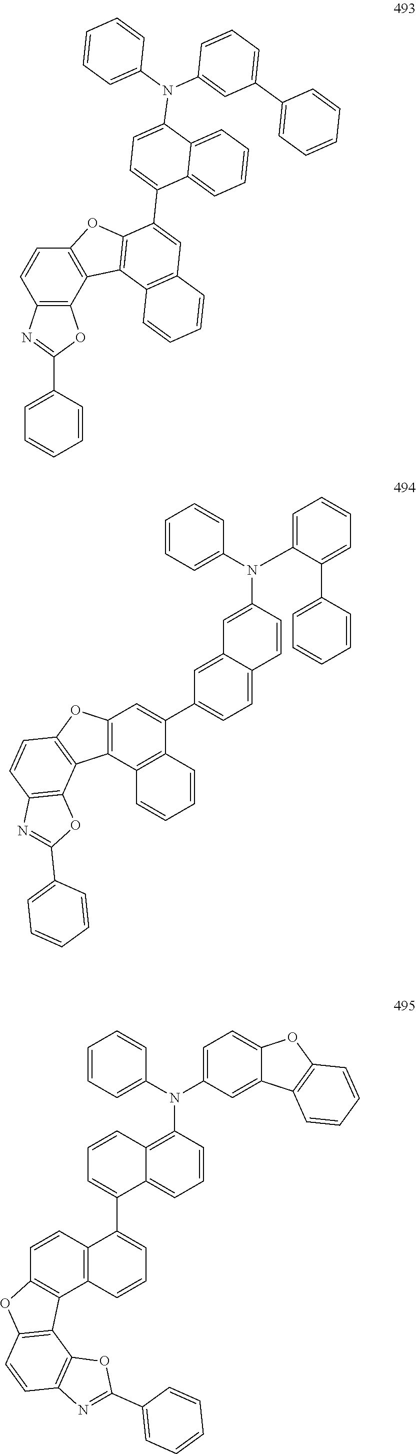 [SMILES: c1ccc(-c2cccc(N(c3ccccc3)c3ccc(-c4cc5ccccc5c5c4oc4ccc6nc(-c7ccccc7)oc6c45)c4ccccc34)c2)cc1 ; c1ccc(-c2nc3ccc4oc5cc(-c6ccc7ccc(N(c8ccccc8)c8ccccc8-c8ccccc8)cc7c6)c6ccccc6c5c4c3o2)cc1 ; c1ccc(-c2nc3ccc4oc5ccc6c(-c7cccc8c(N(c9ccccc9)c9ccc%10oc%11ccccc%11c%10c9)cccc78)cccc6c5c4c3o2)cc1]

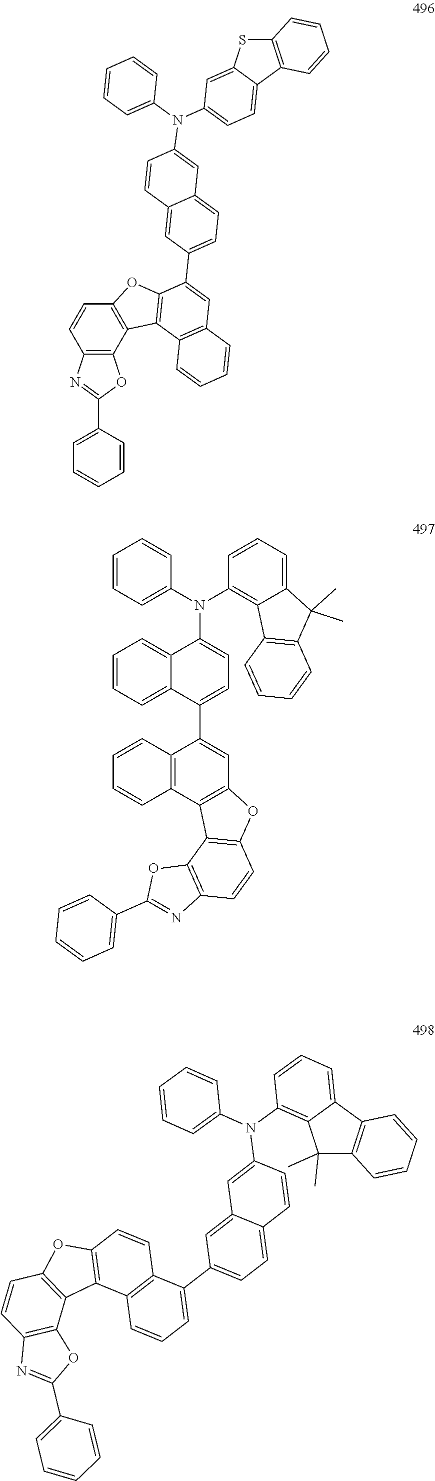 [SMILES: c1ccc(-c2nc3ccc4oc5c(-c6ccc7cc(N(c8ccccc8)c8ccc9c(c8)sc8ccccc89)ccc7c6)cc6ccccc6c5c4c3o2)cc1 ; CC1(C)c2ccccc2-c2c(N(c3ccccc3)c3ccc(-c4cc5oc6ccc7nc(-c8ccccc8)oc7c6c5c5ccccc45)c4ccccc34)cccc21 ; CC1(C)c2ccccc2-c2cccc(N(c3ccccc3)c3ccc4ccc(-c5cccc6c5ccc5oc7ccc8nc(-c9ccccc9)oc8c7c56)cc4c3)c21]

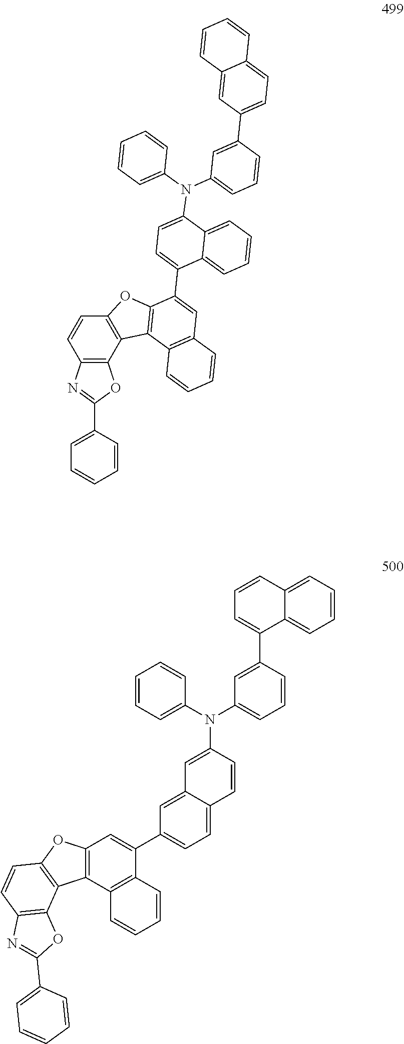 [SMILES: c1ccc(-c2nc3ccc4oc5c(-c6ccc(N(c7ccccc7)c7cccc(-c8ccc9ccccc9c8)c7)c7ccccc67)cc6ccccc6c5c4c3o2)cc1 ; c1ccc(-c2nc3ccc4oc5cc(-c6ccc7ccc(N(c8ccccc8)c8cccc(-c9cccc%10ccccc9%10)c8)cc7c6)c6ccccc6c5c4c3o2)cc1]

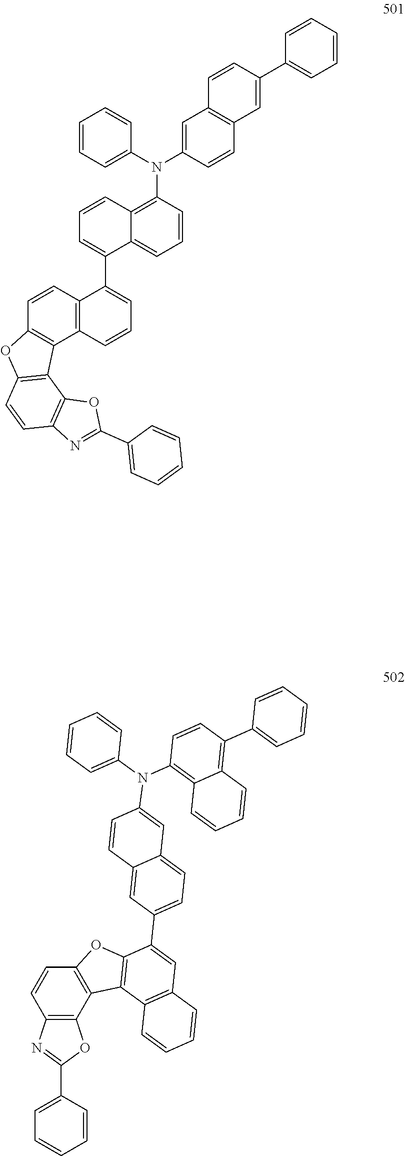 [SMILES: c1ccc(-c2ccc3cc(N(c4ccccc4)c4cccc5c(-c6cccc7c6ccc6oc8ccc9nc(-c%10ccccc%10)oc9c8c67)cccc45)ccc3c2)cc1 ; c1ccc(-c2nc3ccc4oc5c(-c6ccc7cc(N(c8ccccc8)c8ccc(-c9ccccc9)c9ccccc89)ccc7c6)cc6ccccc6c5c4c3o2)cc1]

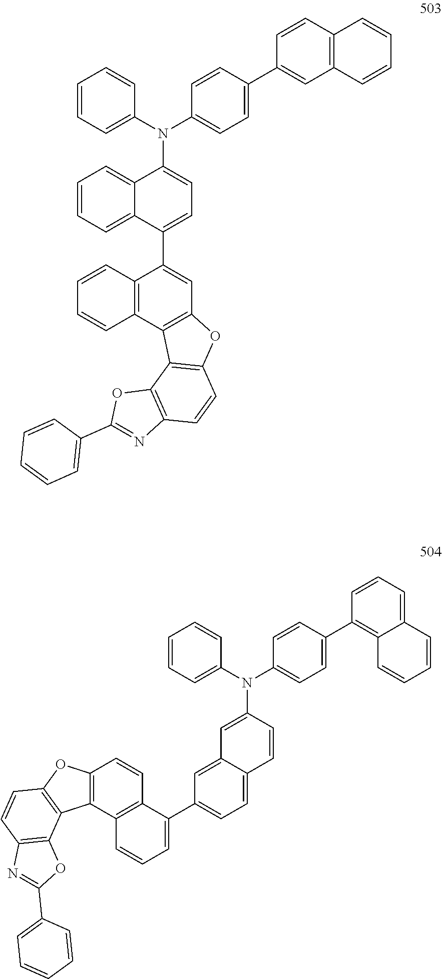 [SMILES: c1ccc(-c2nc3ccc4oc5cc(-c6ccc(N(c7ccccc7)c7ccc(-c8ccc9ccccc9c8)cc7)c7ccccc67)c6ccccc6c5c4c3o2)cc1 ; c1ccc(-c2nc3ccc4oc5ccc6c(-c7ccc8ccc(N(c9ccccc9)c9ccc(-c%10cccc%11ccccc%10%11)cc9)cc8c7)cccc6c5c4c3o2)cc1]

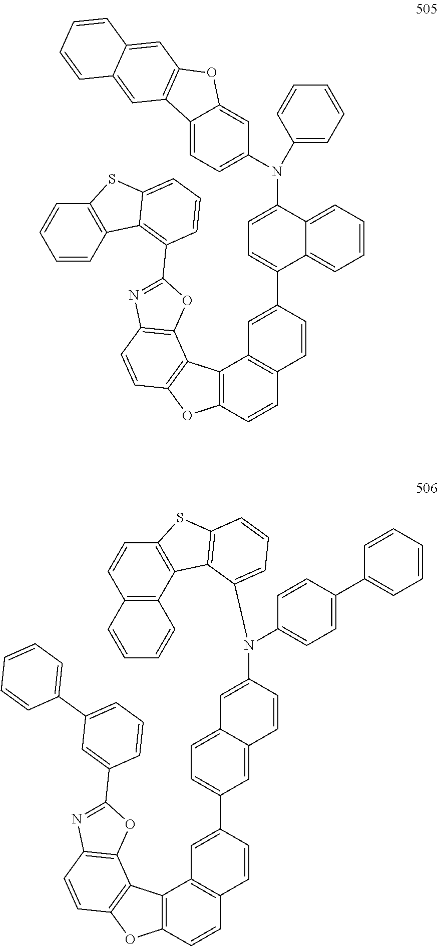 [SMILES: c1ccc(N(c2ccc3c(c2)oc2cc4ccccc4cc23)c2ccc(-c3ccc4ccc5oc6ccc7nc(-c8cccc9sc%10ccccc%10c89)oc7c6c5c4c3)c3ccccc23)cc1 ; c1ccc(-c2ccc(N(c3ccc4cc(-c5ccc6ccc7oc8ccc9nc(-c%10cccc(-c%11ccccc%11)c%10)oc9c8c7c6c5)ccc4c3)c3cccc4sc5ccc6ccccc6c5c34)cc2)cc1]

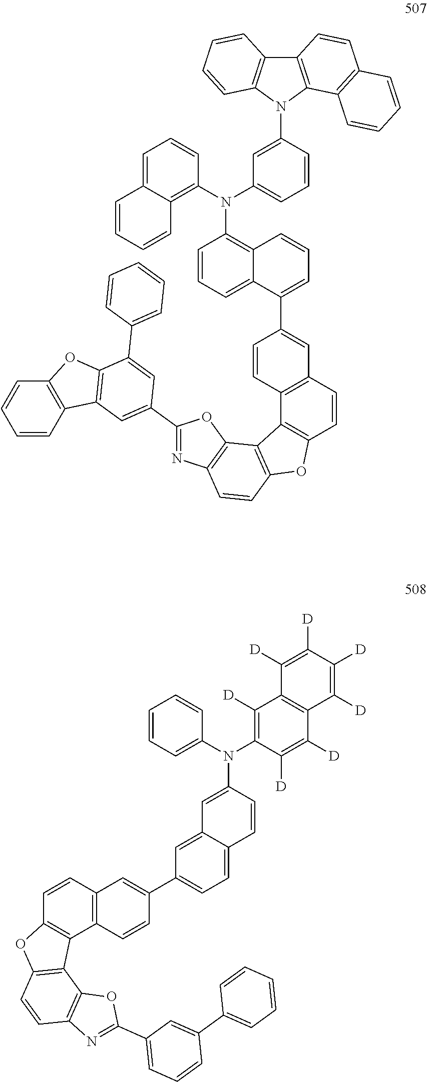 [SMILES: c1ccc(-c2cc(-c3nc4ccc5oc6ccc7cc(-c8cccc9c(N(c%10cccc(-n%11c%12ccccc%12c%12ccc%13ccccc%13c%12%11)c%10)c%10cccc%11ccccc%10%11)cccc89)ccc7c6c5c4o3)cc3c2oc2ccccc23)cc1 ; [2H]c1c([2H])c([2H])c2c([2H])c(N(c3ccccc3)c3ccc4ccc(-c5ccc6c(ccc7oc8ccc9nc(-c%10cccc(-c%11ccccc%11)c%10)oc9c8c76)c5)cc4c3)c([2H])c([2H])c2c1[2H]]

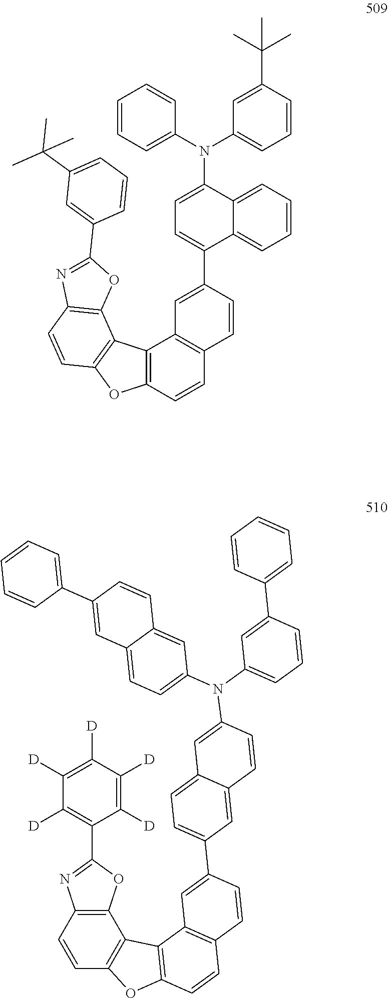 [SMILES: CC(C)(C)c1cccc(-c2nc3ccc4oc5ccc6ccc(-c7ccc(N(c8ccccc8)c8cccc(C(C)(C)C)c8)c8ccccc78)cc6c5c4c3o2)c1 ; [2H]c1c([2H])c([2H])c(-c2nc3ccc4oc5ccc6ccc(-c7ccc8cc(N(c9cccc(-c%10ccccc%10)c9)c9ccc%10cc(-c%11ccccc%11)ccc%10c9)ccc8c7)cc6c5c4c3o2)c([2H])c1[2H]]

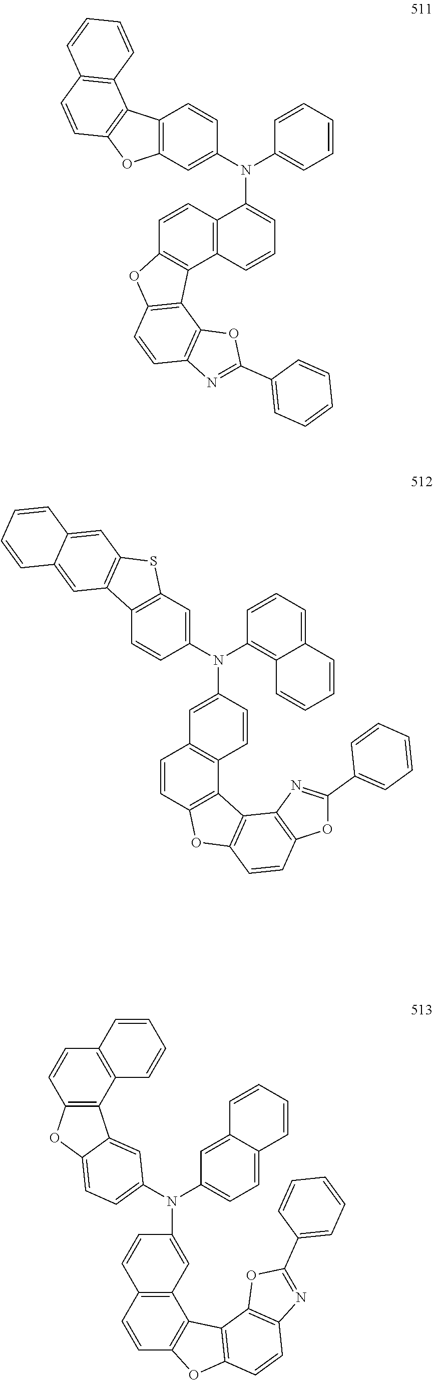 [SMILES: c1ccc(-c2nc3ccc4oc5ccc6c(N(c7ccccc7)c7ccc8c(c7)oc7ccc9ccccc9c78)cccc6c5c4c3o2)cc1 ; c1ccc(-c2nc3c(ccc4oc5ccc6cc(N(c7ccc8c(c7)sc7cc9ccccc9cc78)c7cccc8ccccc78)ccc6c5c43)o2)cc1 ; c1ccc(-c2nc3ccc4oc5ccc6ccc(N(c7ccc8ccccc8c7)c7ccc8oc9ccc%10ccccc%10c9c8c7)cc6c5c4c3o2)cc1]

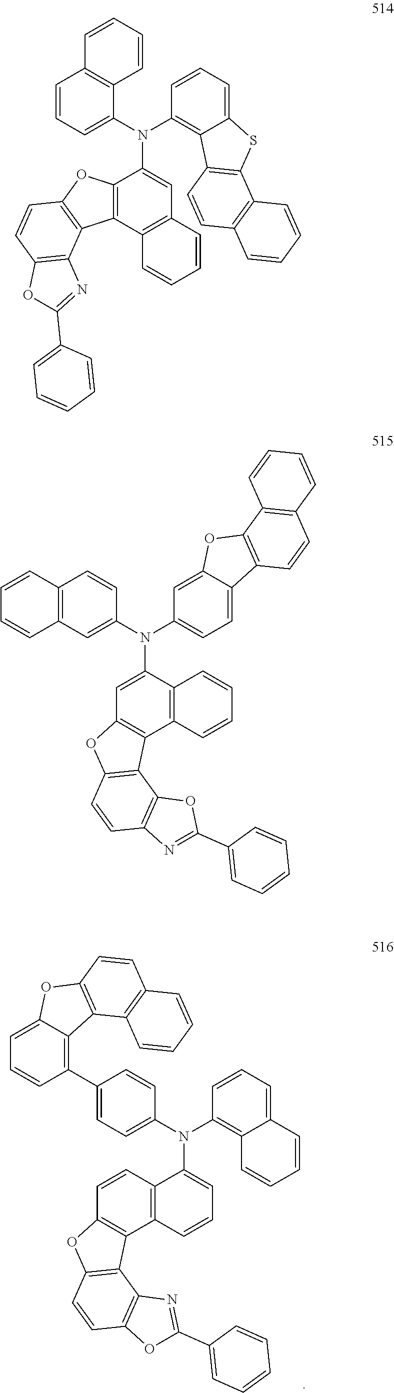 [SMILES: c1ccc(-c2nc3c(ccc4oc5c(N(c6cccc7ccccc67)c6cccc7sc8c9ccccc9ccc8c67)cc6ccccc6c5c43)o2)cc1 ; c1ccc(-c2nc3ccc4oc5cc(N(c6ccc7ccccc7c6)c6ccc7c(c6)oc6c8ccccc8ccc76)c6ccccc6c5c4c3o2)cc1 ; c1ccc(-c2nc3c(ccc4oc5ccc6c(N(c7ccc(-c8cccc9oc%10ccc%11ccccc%11c%10c89)cc7)c7cccc8ccccc78)cccc6c5c43)o2)cc1]

13. An organic electroluminescent device, comprising an anode and a cathode disposed opposite, and a functional layer disposed between the anode and the cathode, wherein the functional layer comprises the heterocyclic compound according to claim 1.

14. The organic electroluminescent device according to claim 13, wherein the functional layer comprises an organic luminescent layer, and the organic luminescent layer comprises the heterocyclic compound; and/or
the functional layer further comprises a hole adjustment layer, and the hole adjustment layer comprises the heterocyclic compound.

15. An electronic apparatus, comprising the organic electroluminescent device according to claim 13.
